(19) United States
(12) Patent Application Publication
(10) Pub. No.: US 2021/0040111 A1
(43) Pub. Date: Feb. 11, 2021

(54) DRUGS TO TREAT OCULAR DISORDERS

(71) Applicant: Graybug Vision, Inc., Redwood City, CA (US)

(72) Inventors: Ming Yang, Lutherville-Timonium, MD (US); John G. Bauman, El Sobrante, CA (US); Jinzhong Zhang, Redwood City, CA (US); Nu Hoang, Annapolis, MD (US); Jeffrey L. Cleland, San Carlos, CA (US)

(73) Assignee: Graybug Vision, Inc., Redwood City, CA (US)

(21) Appl. No.: 17/077,853

(22) Filed: Oct. 22, 2020

Related U.S. Application Data
(63) Continuation of application No. PCT/US2019/029416, filed on Apr. 26, 2019.
(60) Provisional application No. 62/663,111, filed on Apr. 26, 2018.

(51) Int. Cl. C07D 495/04; A61K 9/00; C07D 307/52; C07C 311/16; C07D 207/06; C07D 417/04; C07D 417/14

(52) U.S. Cl. CPC C07D 495/04; A61K 9/0019; A61K 9/0048; C07D 417/14; C07C 311/16; C07D 207/06; C07D 417/04; C07D 307/52

(57) ABSTRACT

The present invention provides new prodrugs of therapeutically active loop diuretics, including oligomeric prodrugs, and compositions to treat medical disorders, for example, ocular disorders such as glaucoma, a disorder or abnormality related to an increase in intraocular pressure (IOP), a disorder requiring neuroprotection, age-related macular degeneration, or diabetic retinopathy.

### CROSS-REFERENCE TO RELATED APPLICATIONS

This application is a continuation of International Patent Application No. PCT/US2019/029416, filed in the U.S. Receiving Office on Apr. 26, 2019, which claims the benefit of provisional U.S. Application No. 62/663,111, filed Apr. 26, 2018. The entirety of each these applications is hereby incorporated by reference herein for all purposes.

### BACKGROUND

The eye is a complex organ with unique anatomy and physiology. The structure of the eye can be divided into two parts, the anterior and posterior. The cornea, conjunctiva, aqueous humor, iris, ciliary body and lens are in the anterior portion. The posterior portion includes the sclera, choroid, retinal pigment epithelium, neural retina, optic nerve and vitreous humor. The most prevalent diseases affecting the posterior segment of the eye are dry and wet age-related macular degeneration (AMD) and diabetic retinopathy. The most important diseases affecting the anterior segment include glaucoma, allergic conjunctivitis, anterior uveitis and cataracts. Glaucoma, which damages the eye's optic nerve, is a leading cause of vision loss and blindness.

To address issues of ocular delivery, a large number of types of delivery systems have been devised, including conventional (solution, suspension, emulsion, ointment, inserts and gels); vesicular (liposomes, exosomes, niosomes, discomes and pharmacosomes); advanced materials (scleral plugs, gene delivery, siRNA and stem cells); and, controlled release systems (implants, hydrogels, dendrimers, iontophoresis, collagen shields, polymeric solutions, therapeutic contact lenses, cyclodextrin carriers, microneedles and microemulsions and particulates (microparticles and nanoparticles)).

Topical drops are widely used non-invasive routes of drug administration to treat anterior ocular diseases due to their non-invasiveness and convenience. Typical routes of drug delivery to the eye are topical, systemic, subconjunctival, intravitreal, punctal, intrasceral, transscleral, anterior or posterior sub-Tenon's, suprachoroidal, choroidal, subchoroidal, and subretinal.
Drug delivery to the posterior area of the eye usually requires a different mode of administration from topical drops, and is typically achieved via an intravitreal injection, periocular injection or systemic administration. Systemic administration is not preferred given the ratio of volume of the eye to the entire body and thus unnecessary potential systemic toxicity. Therefore, intravitreal injections are currently the most common form of drug administration for posterior disorders. However, intravitreal injections are also associated with risk due to the common side effect of inflammation to the eye caused by administration of foreign material to this sensitive area, endophthalmitis, hemorrhage, retinal detachment and poor patient compliance.

Transscleral delivery with periocular administration is seen as an alternative to intravitreal injections, however, ocular barriers such as the sclera, choroid, retinal pigment epithelium, lymphatic flow and general blood flow compromise efficacy.

To treat ocular diseases, and in particular disease of the posterior chamber, the drug must be delivered in an amount and for a duration to achieve efficacy.

Patent applications that describe loop diuretic prodrugs include WO2006/047466 assigned to Duke University titled “Ophthalmological Drugs”; U.S. Pat. No. 5,565,434 assigned to the University of Iowa Research Foundation titled “Hexose and Pentose Prodrugs of Ethacrynic acid”; WO 2016/118506 titled “Compositions for the Sustained Release of Anti-Glaucoma Agents to control Intraocular Pressure” assigned to the Johns Hopkins University; U.S. Pat. No. 4,661,515 titled “Compounds having Angiotensin Converting Enzyme Inhibitory Activity and Diuretic Activity” assigned to USV Pharmaceutical Corporation; and, CN 103610669 titled “Bis-(p-alkoxy benzene acrylketone) like glutathione-S-transferase potential inhibitor”. Neurotherapeutics Pharma LLC has filed applications disclosing prodrugs of loop diuretics, including WO 2007/047698 tilted “Methods and Compositions for the Treatment of Neuropsychiatric and Addictive Disorders”; WO 2010/085352 titled “Bumetanide, Furosemide, Piretanide, Azosemide, and Torsemide Analogs, Compositions, and Method of Use”; WO 2013/059648 titled “2, 3, 5 Trisubstituted Aryl and Heteroaryl Amino Derivatives, Compositions, and Methods of Use”, Chinese patent application No. CN 103897174 titled “Novel polymer containing ethacrynic acid structure, preparation method thereof and applications thereof”, and Chinese patent No. titled “Novel compound with ethacrynic acid structure as well as preparation method and application of novel compound”.

U.S. Patent application 2010/227865 titled “Oligomer-Beta Blocker Conjugates” describes beta-blocker mono prodrugs. Johns Hopkins University has filed a number of patents claiming formulations for ocular injections including WO2013/138343 titled “Controlled Release Formulations for the Delivery of HIF-1 Inhibitors”, WO2013/138346 titled “Non-linear Multiblock Copolymer-drug Conjugates for the Delivery of Active Agents”, WO2011/106702 titled “Sustained Delivery of Therapeutic Agents to an Eye Compartment”, WO2016/025215 titled “Glucorticoid-loaded Nanoparticles for Prevention of Corneal Allograft Rejection and Neovascularization”, WO2016/100392 titled “Sunitinib Formulations and Methods for Use Thereof in Treatment of Ocular Disorders”, WO2016/100380 titled “Sunitinib Formulation and Methods for Use Thereof in Treatment of Glaucoma”, WO2016/118506 titled “Compositions for the Sustained Release of Anti-Glaucoma Agents to Control Intraocular Pressure”, WO2013/166385 titled “Nanocrystals, Compositions, and Methods that Aid Particle Transport in Mucus”, WO2005/072710 titled “Drug and Gene Carrier Particles that Rapidly move Through Mucus Barriers,” WO2008/030557 titled “Compositions and Methods for Enhancing Transport through Mucus”, WO2012/061703 titled “Compositions and Methods Relating to Reduced Mucoadhesion,” WO2012/039979 titled “Large Nanoparticles that Penetrate Tissue,” WO2012/109363 titled “Mucus Penetrating Gene Carriers”, WO2013/090804 titled “Biodegradable Stealth Nanoparticles Prepared by a Novel Self-Assembly Emulsification Method,” WO2013/110028 titled “Nanoparticles Formulations with Enhanced Mucosal Penetration”, and WO2013/166498 titled “Lipid-based Drug Carriers for Rapid Penetration through Mucus Linings”.

GrayBug Vision, Inc. discloses prodrugs for the treatment of ocular therapy in U.S. Pat. Nos. 9,808,531; 10,098,965; 10,117,950; 9,956,302; 10,111,964; and, 10,159,747; US Application No. US 2019-0060474; and PCT application WO 2018/175922. Aggregating microparticles for ocular therapy are described in WO/2017/083779 and WO/2018/209155.

There remains a need to deliver effective therapies to the eye, including those that can reduce ocular pressure. Therefore, the object of this invention is to provide new compounds, compositions and methods to treat ocular disorders, including that reduce intraocular pressure (IOP).

### SUMMARY

The present invention provides new prodrugs, including oligomeric prodrugs, and compositions thereof of the specific loop diuretics Furosemide, Bumetanide, Piretanide, or Ozolinone to provide therapies that are advantageous for ocular delivery of these drugs.

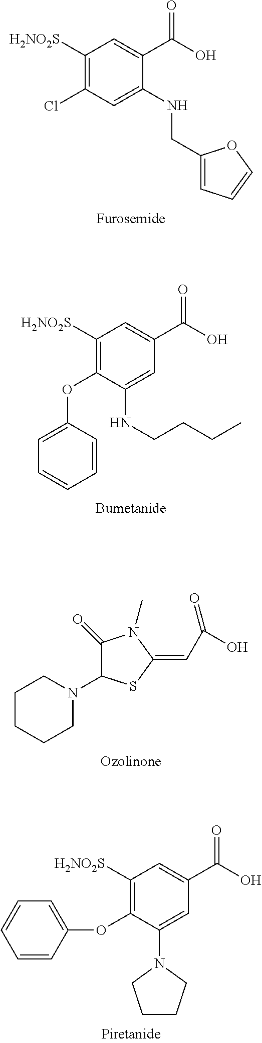 [SMILES: NS(=O)(=O)c1cc(C(=O)O)c(NCc2ccco2)cc1Cl ; CCCCNc1cc(C(=O)O)cc(S(N)(=O)=O)c1Oc1ccccc1 ; CN1C(=O)C(N2CCCCC2)S/C1=C/C(=O)O ; NS(=O)(=O)c1cc(C(=O)O)cc(N2CCCC2)c1Oc1ccccc1]

In one embodiment, the invention is an active compound or pharmaceutically acceptable salt of Formula I, Formula II, Formula III, Formula IV, Formula IV′ Formula V, Formula VI, Formula VII, Formula VIII, Formula VIII′, Formula IX, Formula X, Formula XI, Formula XII, Formula XII′, Formula XIII, Formula XIV, Formula XV, Formula XVI, Formula XVI′, Formula XVII, Formula XVIII, Formula XIX, Formula XX, Formula XX′, Formula XXI, Formula XXII, Formula XXIII, Formula XXIV, or Formula XXIV′.

In one embodiment, the invention is a method for delivering an active prodrug to the eye that includes presenting it as discussed herein in a controlled delivery system, for example a microparticle or nanoparticle, that allows for sustained delivery.

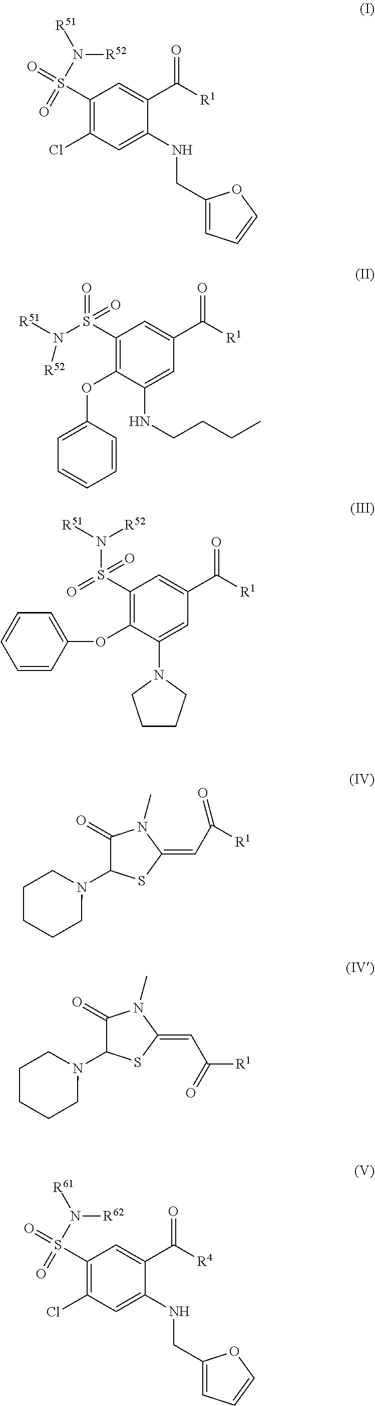

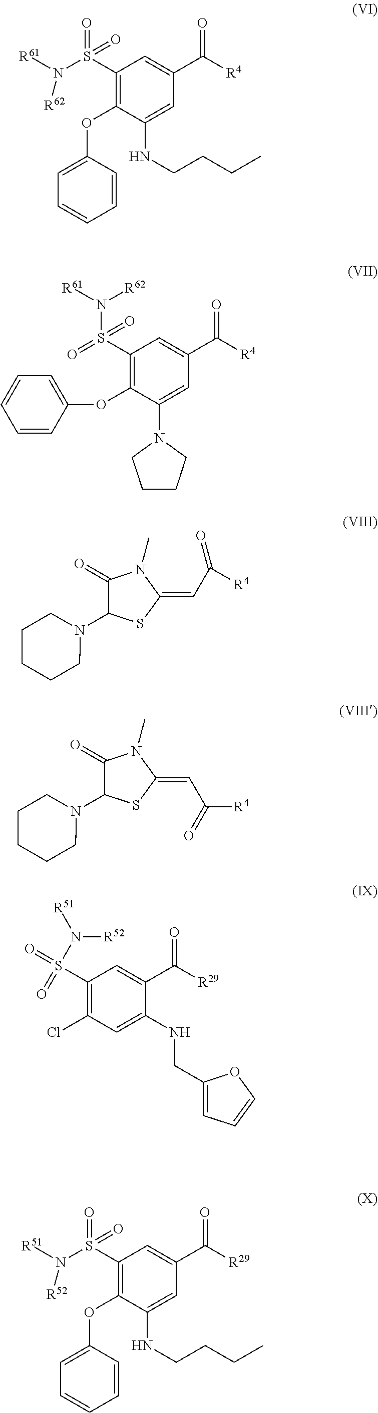

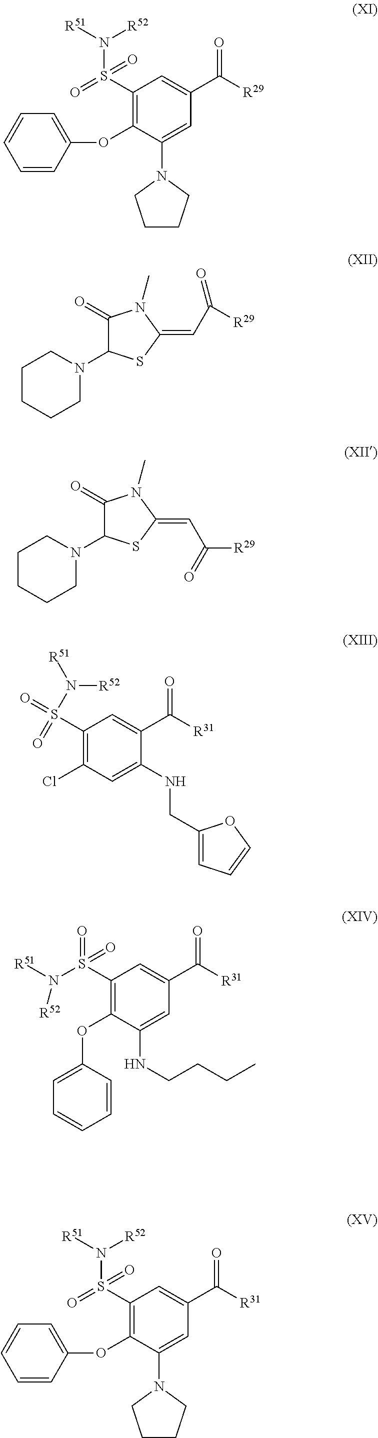

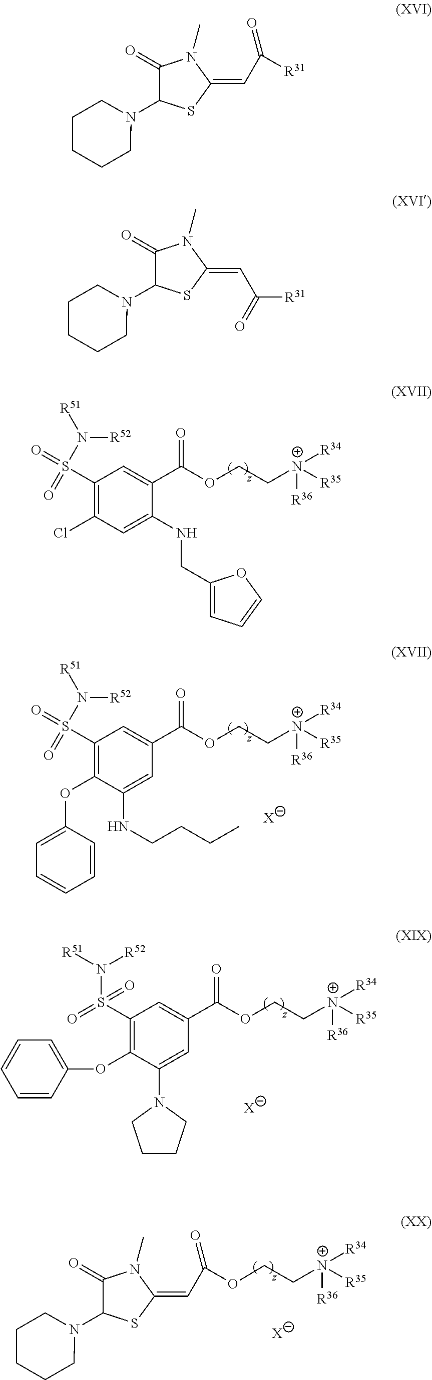

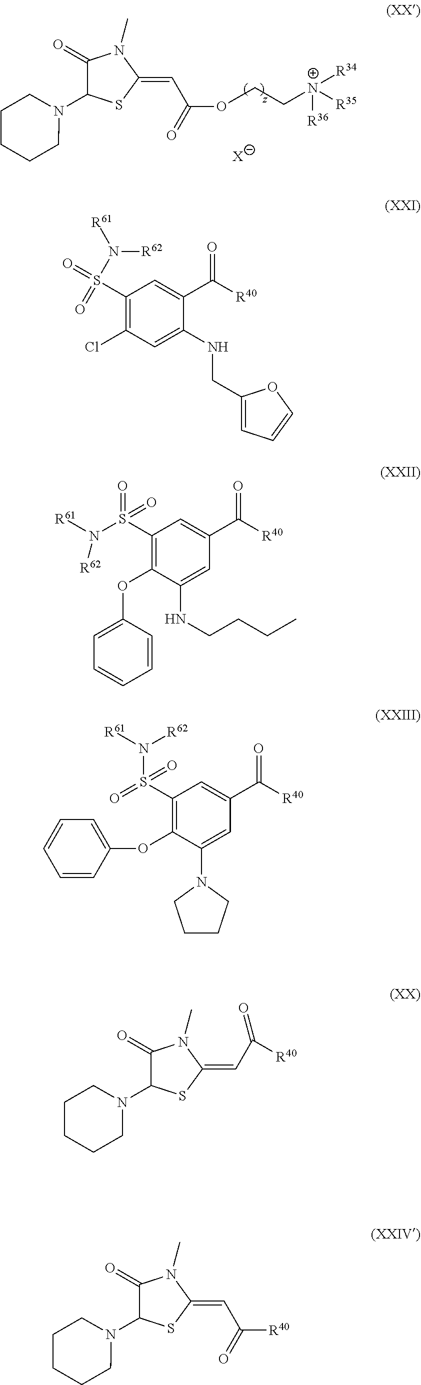

The active therapeutic agent delivered in modified form is selected from the loop diuretics Furosemide, Bumetanide, Piretanide, and Ozolinone, which is the metabolite of Etozolin.

Any of the compounds or pharmaceutically acceptable salts thereof can be administered in an immediate or controlled delivery system as desired to achieve the appropriate effect. The compound, for example, can be administered systemically, topically, parentally, intravenously, subcutaneously, intramuscularly, transdermally, buccally, or sublingually in an effective amount to treat a disorder that can be treated with a loop diuretic.

The compounds of the invention can be used for the controlled administration of active compounds to the eye, over a period of at least two, three, four, five or six months or more in a manner that maintains at least a concentration in the eye that is effective for the disorder to be treated. In some embodiments, the prodrug is provided in a microparticle, microcapsule, vesicle, reservoir, or nanoparticle. In one embodiment, the drug is administered in a polymeric formulation that provides a controlled release that is linear. In another embodiment, the release is not linear; however, even the lowest concentration of release over the designated time period is at or above a therapeutically effective dose. In one embodiment, this is achieved by formulating a hydrophobic prodrug of the invention in a polymeric delivery material such as a polymer or copolymer that includes moieties of at least lactic acid, glycolic acid, propylene oxide or ethylene oxide. In a particular embodiment, the polymeric delivery system includes PLGA, PLA or PGA with or without covalently attached or admixed polyethylene glycol. For example, the hydrophobic drug may be delivered in a mixture of PLGA and PLGA-PEG, PEG, PLA, or PLA-PEG. The hydrophobic drug may be delivered in a mixture of PLA and PLGA-PEG, PEG, PLGA, or PLA-PEG.

In certain embodiments, the prodrug of the present invention is delivered in a microparticle or nanoparticle that is a blend of two polymers, for example (i) a PLGA polymer or PLA polymer as described herein and (ii) a PLGA-PEG or PLA-PEG copolymer. In another embodiment, the microparticle or nanoparticle is a blend of three polymers, such as, for example, (i) a PLGA polymer; (ii) a PLA polymer; and, (iii) a copolymer of PLGA-PEG or PLA-PEG. In an additional embodiment, the microparticle or nanoparticle is a blend of (i) a PLA polymer; (ii) a PLGA polymer; (iii) a PLGA polymer that has a different ratio of lactide and glycolide monomers than the PLGA in (ii); and, (iv) a PLGA-PEG or PLA-PEG copolymer. Any ratio of lactide and glycolide in the PLGA can be used that achieves the desired therapeutic effect. In certain illustrative non-limiting embodiments, the ratio of PLA to PLGA by weight in a polymer blend as described is 77/22, 69/30, 49/50, 54/45, 59/40, 64/35, 69/30, 74/25, 79/20, 84/15, 89/10, 94/5, or 99/1.

In certain embodiments, a blend of three polymers that has (i) PLA (ii) PLGA (iii) PLGA with a different ratio of lactide and glycolide monomers than PLGA in (ii) wherein the ratio by weight is 74/20/5 by weight, 69/20/10 by weight, 69/25/5 by weight, or 64/20/15 by weight. In certain embodiments, the PLGA in (ii) has a ratio of lactide to glycolide of 85/15, 75/25, or 50/50. In certain embodiments the PLGA in (iii) has a ratio of lactide to glycolide of 85/15, 75/25, or 50/50.

In certain aspects, the drug may be delivered in a blend of PLGA or PLA and PEG-PLGA, including but not limited to (i) PLGA+approximately by weight 1% PEG-PLGA or (ii) PLA+approximately by weight 1% PEG-PLGA. In certain aspects, the drug may be delivered in a blend of (iii) PLGA/PLA+approximately by weight 1% PEG-PLGA. In certain embodiments, the blend of PLA, PLGA, or PLA/PGA with PLGA-PEG contains approximately from about 0.5% to about 10% by weight of a PEG-PLGA, from about 0.5% to about 5% by weight of a PEG-PLGA, from about 0.5% to about 4% by weight of a PEG-PLGA, from about 0.5% to about 3% by weight of a PEG-PLGA, from about 1.0% to about 3.0% by weight of a PEG-PLGA, from about 0.1% to about 10% of a PEG-PLGA, from about 0.1% to about 5% of a PEG-PLGA, from about 0.1% to about 1% PEG-PLGA, or from about 0.1% to about 2% PEG-PLGA.

In certain non-limiting embodiments, the ratio by weight percent of PLGA to PEG-PLGA in a two polymer blend as described is about or at least about 40/1, 45/1, 50/1, 55/1, 60/1, 65/1, 70/1, 75/1, 80/1, 85/1, 90/1, 95/1, 96/1, 97/1, 98/1, 99/1. The PLGA can be acid or ester capped. In non-limiting aspects, the drug can be delivered in a two polymer blend of PLGA75:25 4A+approximately 1% PEG-PLGA50:50; PLGA85:15 5A+approximately 1% PEG-PLGA5050; PLGA75:25 6E+approximately 1% PEG-PLGA50:50; or, PLGA50:50 2A+approximately 1% PEG-PLGA50:50.

In certain non-limiting embodiments, the ratio by weight percent of PLA/PLGA-PEG in a polymer blend as described is about or at least about 40/1, 45/1, 50/1, 55/1, 60/1, 65/1, 70/1, 75/1, 80/1, 85/1, 90/1, 95/1, 96/1, 97/1, 98/1, 99/1. The PLA can be acid capped or ester capped. In cetain aspects, the PLA is PLA 4.5A. In non-limiting aspects, the drug is delivered in a blend of PLA 4.5A+1% PEG-PLGA.

The PEG segment of the PEG-PLGA may have, for example, in non-limiting embodiments, a molecular weight of at least about or about 1 kDa, 2 kDa, 3 kDa, 4 kDa, 5 kDa, 6 kDa, 7 kDa, 8 kDa, 9 kDa, or 10 kDa, and typically not greater than 10 kDa, 15 kDa, 20 kDa, or 50 kDa, or in some embodiments, 6 kDa, 7 kDa, 8 kDa, or 9 kDa. In certain embodiment, the PEG segment of the PEG-PLGA has a molecular weight between about 3 kDa and about 7 kDa or between about 2 kDa and about 7 kDa. Non-limiting examples of the PLGA segment of the PEG-PLGA is PLGA50:50, PLGA75:25, or PLGA85:15. In one embodiment, the PEG-PLGA segment is PEG (5 kDa)-PLGA50:50.

When the drug is delivered in a blend of PLGA+PEG-PLGA, any ratio of lactide and glycolide in the PLGA or the PLGA-PEG can be used that achieves the desired therapeutic effect. Non-limiting illustrative embodiments of the ratio of lactide/glycolide in the PLGA or PLGA-PEG are about or at least about 5/95, 10/90, 15/85, 20/80, 25/75, 30/70, 35/65, 40/60, 45/55, 50/50, 55/45, 60/40, 65/35, 70/30, 75/25, 80/20, 85/15, 90/10, or 95/5. In one embodiment, the PLGA is a block co-polymer, for example, diblock, triblock, multiblock, or star-shaped block. In one embodiment, the PLGA is a random co-polymer. In certain aspects, the PLGA is PLGA75:25 4A; PLGA85:15 5A; PLGA75:25 6E; or, PLGA50:50 2A.

In another embodiment, the polymer includes a polyethylene oxide (PEO) or polypropylene oxide (PPO). In certain aspects, the polymer can be a random, block, diblock, triblock or multiblock copolymer (for example, a polylactide, a polylactide-co-glycolide, polyglycolide or Pluronic). For injection into the eye, the polymer is pharmaceutically acceptable and typically biodegradable so that it does not have to be removed.

The decreased rate of release of the active material to the ocular compartment may result in decreased inflammation, which has been a significant side effect of ocular therapy to date.

It is also important that the decreased rate of release of the drug while maintaining efficacy over an extended time of up to 2, 3, 4, 5 or 6 months be achieved using a particle that is small enough for administration through a needle without causing significant damage or discomfort to the eye and not to give the illusion to the patient of black spots floating in the eye. This typically means the controlled release particle should be less than approximately 300, 250, 200, 150, 100, 50, 45, 40, 35, or 30 μm, such as less than approximately 30, 29, 28, 27, 26, 25, 24, 23, 22 21, or 20 μm. In one aspect, the particles do not agglomerate in vivo to form larger particles, but instead in general maintain their administered size and decrease in size over time.

The hydrophobicity of the conjugated drug can be measured using a partition coefficient (P; such as Log P in octanol/water), or distribution coefficient (D; such as Log D in octanol/water) according to methods well known to those of skill in the art. Log P is typically used for compounds that are substantially un-ionized in water and Log D is typically used to evaluate compounds that ionize in water. In certain embodiments, the conjugated derivatized drug has a Log P or Log D of greater than approximately 2.5, 3, 3.5, 4, 4.5, 5, 5.5 or 6. In other embodiments, the conjugated derivatized drug has a Log P or Log D which is at least approximately 1, 1.5, 2, 2.5, 3, 3.5 or 4 Log P or Log D units, respectively, higher than the parent hydrophilic drug.

This invention includes an active compound of Formula I, Formula II, Formula III, Formula IV, Formula IV′ Formula V, Formula VI, Formula VII, Formula VIII, Formula VIII′, Formula IX, Formula X, Formula XI, Formula XII, Formula XII′, Formula XIII, Formula XIV, Formula XV, Formula XVI, Formula XVI′, Formula XVII, Formula XVIII, Formula XIX, Formula XX, Formula XX′, Formula XXI, Formula XXII, Formula XXIII, Formula XXIV, or Formula XXIV′ or a pharmaceutically acceptable salt or composition thereof. In one embodiment, an active compound or its salt or composition, as described herein, is used to treat a medical disorder which is glaucoma, a disorder mediated by carbonic anhydrase, a disorder mediated by a Rho-associated kinase, a disorder mediated by a dual leucine zipper kinase, a disorder mediated by VEGF, a disorder or abnormality related to an increase in intraocular pressure (IOP), a disorder mediated by nitric oxide synthase (NOS), or a disorder requiring neuroprotection such as to regenerate/repair optic nerves. In another embodiment more generally, the disorder treated is allergic conjunctivitis, anterior uveitis, cataracts, dry or wet age-related macular degeneration (AMD), geographic atrophy, or diabetic retinopathy. In one embodiment, an active compound or its salt or composition, as described herein, is used to decrease IOP. In one embodiment, an active compound or its salt or composition is used to treat optic nerve damage associated with IOP.

In other embodiments, the parent drug Furosemide, Bumetanide, Piretanide or Ozolinone in free form (i.e., not as a prodrug) or its pharmaceutically acceptable salt or a combination thereof or a combination with one of the prodrugs of described herein is provided in an effective amount to the patient in a microparticle for ocular delivery. In another embodiment, the parent drug Furosemide, Bumetanide, Piretanide or Ozolinone or its pharmaceutically acceptable salt or a combination thereof or a combination with one of the prodrugs of described herein is provided to the patient by administration to the eye via intravitreal, intrastromal, intracameral, sub-tenon, sub-retinal, retro-bulbar, peribulbar, suprachoroidal, choroidal, subchoroidal, conjunctival, episcleral, posterior juxtascleral, circumcorneal, or tear duct injection in combination with one or more pharmaceutically acceptable carriers. In certain aspects, furosemide, bumetanide, or piretanide are administered in a site that is not near the trabecular meshwork. In certain aspects, etozolin is administered via subconjunctival injection.

Compounds of Formula I are single agent prodrugs of the loop diuretic Furosemide.

In alternative embodiments, compounds of Formula I are pharmaceutically acceptable salts of hydrophobic prodrugs of Furosemide.

Compounds of Formula II are single agent prodrugs of the loop diuretic Bumetanide.

In alternative embodiments, compounds of Formula II are pharmaceutically acceptable salts of hydrophobic prodrugs of Bumetanide.

Compounds of Formula III are single agent prodrugs of the loop diuretic Piretanide.

In alternative embodiments, compounds of Formula III are pharmaceutically acceptable salts of hydrophobic prodrugs of Piretanide.

Compounds of Formula IV and Formula IV′ are single agent prodrugs of Ozolinone, the active metabolite of the loop diuretic Etozolin.

In alternative embodiments, compounds of Formula IV and Formula IV′ are pharmaceutically acceptable salts of hydrophobic prodrugs of Ozolinone, the active metabolite of the loop diuretic Etozolin.

Compounds of Formula V are pharmaceutically acceptable salts of prodrug conjugates of Furosemide and Brimonidine allowing release of both compounds in the eye. In one embodiment both compounds are released concurrently.

In alternative embodiments, compounds of Formula V are prodrug conjugates of a carbonic anhydrase inhibitor and Furosemide allowing release of both compounds in the eye. In one embodiment both compounds are released concurrently.

In alternative embodiments, compounds of Formula V are prodrug conjugates of a dual leucine zipper kinase inhibitor and Furosemide allowing release of both compounds in the eye. In one embodiment both compounds are released concurrently.

In alternative embodiments, compounds of Formula V are prodrug conjugates of Furosemide and a Sunitinib derivative allowing release of both compounds in the eye. In one embodiment both compounds are released concurrently.

In alternative embodiments, compounds of Formula V are single agent prodrug conjugates of Furosemide and a prostaglandin derivative allowing release of both compounds in the eye. In one embodiment both compounds are released concurrently.

In alternative embodiments, compounds of Formula V are single agent prodrug conjugates of a ROCK inhibitor and Furosemide allowing release of both compounds in the eye. In one embodiment both compounds are released concurrently.

In alternative embodiments, compounds of Formula V are single agent prodrug conjugates of Timolol and Furosemide allowing release of both compounds in the eye. In one embodiment both compounds are released concurrently.

Compounds of Formula VI are pharmaceutically acceptable salts of prodrug conjugates of Bumetanide and Brimonidine allowing release of both compounds in the eye. In one embodiment both compounds are released concurrently.

In alternative embodiments, compounds of Formula VI are prodrug conjugates of a carbonic anhydrase inhibitor and Bumetanide allowing release of both compounds in the eye. In one embodiment both compounds are released concurrently.

In alternative embodiments, compounds of Formula VI are prodrug conjugates of a dual leucine zipper kinase inhibitor and Bumetanide allowing release of both compounds in the eye. In one embodiment both compounds are released concurrently.

In alternative embodiments, compounds of Formula VI are prodrug conjugates of Bumetanide and a Sunitinib derivative allowing release of both compounds in the eye. In one embodiment both compounds are released concurrently.

In alternative embodiments, compounds of Formula VI are single agent prodrug conjugates of Bumetanide and a prostaglandin derivative allowing release of both compounds in the eye. In one embodiment both compounds are released concurrently.

In alternative embodiments, compounds of Formula VI are single agent prodrug conjugates of a ROCK inhibitor and Bumetanide allowing release of both compounds in the eye. In one embodiment both compounds are released concurrently.

In alternative embodiments, compounds of Formula VI are single agent prodrug conjugates of Timolol and Bumetanide allowing release of both compounds in the eye. In one embodiment both compounds are released concurrently.

Compounds of Formula VII are pharmaceutically acceptable salts of prodrug conjugates of Piretanide and Brimonidine allowing release of both compounds in the eye. In one embodiment both compounds are released concurrently.

In alternative embodiments, compounds of Formula VII are prodrug conjugates of a carbonic anhydrase inhibitor and Piretanide allowing release of both compounds in the eye. In one embodiment both compounds are released concurrently.

In alternative embodiments, compounds of Formula VII are prodrug conjugates of a dual leucine zipper kinase inhibitor and Piretanide allowing release of both compounds in the eye. In one embodiment both compounds are released concurrently.

In alternative embodiments, compounds of Formula VII are prodrug conjugates of Piretanide and a Sunitinib derivative allowing release of both compounds in the eye. In one embodiment both compounds are released concurrently.

In alternative embodiments, compounds of Formula VII are single agent prodrug conjugates of Piretanide and a prostaglandin derivative allowing release of both compounds in the eye. In one embodiment both compounds are released concurrently.

In alternative embodiments, compounds of Formula VII are single agent prodrug conjugates of a ROCK inhibitor and Piretanide allowing release of both compounds in the eye. In one embodiment both compounds are released concurrently.

In alternative embodiments, compounds of Formula VII are single agent prodrug conjugates of Timolol and Piretanide allowing release of both compounds in the eye. In one embodiment both compounds are released concurrently.

Compounds of Formula VIII and Formula VIII′ are pharmaceutically acceptable salts of prodrug conjugates of Ozolinone and Brimonidine allowing release of both compounds in the eye. In one embodiment both compounds are released concurrently.

In alternative embodiments, compounds of Formula VIII and Formula VIII′ are prodrug conjugates of a carbonic anhydrase inhibitor and Ozolinone allowing release of both compounds in the eye. In one embodiment both compounds are released concurrently.

In alternative embodiments, compounds of Formula VIII and Formula VIII′ are prodrug conjugates of a dual leucine zipper kinase inhibitor and Ozolinone allowing release of both compounds in the eye. In one embodiment both compounds are released concurrently.

In alternative embodiments, compounds of Formula VIII and Formula VIII′ are prodrug conjugates of Ozolinone and a Sunitinib derivative allowing release of both compounds in the eye. In one embodiment both compounds are released concurrently.

In alternative embodiments, compounds of Formula VIII and Formula VIII′ are single agent prodrug conjugates of Ozolinone and a prostaglandin derivative allowing release of both compounds in the eye. In one embodiment both compounds are released concurrently.

In alternative embodiments, compounds of Formula VIII and Formula VIII′ are single agent prodrug conjugates of a ROCK inhibitor and Ozolinone allowing release of both compounds in the eye. In one embodiment both compounds are released concurrently.

In alternative embodiments, compounds of Formula VIII and Formula VIII′ are single agent prodrug conjugates of Timolol and Ozolinone allowing release of both compounds in the eye. In one embodiment both compounds are released concurrently.

Compounds of Formula IX are pharmaceutically acceptable salts of prodrug conjugates of Furosemide and Bumetanide allowing release of both compounds in the eye. In one embodiment both compounds are released concurrently.

In alternative embodiments, compounds of Formula IX are prodrug conjugates of Furosemide and Piretanide allowing release of both compounds in the eye. In one embodiment both compounds are released concurrently.

In alternative embodiments, compounds of Formula IX are prodrug conjugates of Furosemide and Ozolinone allowing release of both compounds in the eye. In one embodiment both compounds are released concurrently.

In alternative embodiments, compounds of Formula IX are prodrug conjugates of Furosemide and ethacrynic acid allowing release of both compounds in the eye. In one embodiment both compounds are released concurrently.

In alternative embodiments, compounds of Formula IX are prodrug conjugates of Furosemide allowing release of two units of Furosemide in the eye. In one embodiment both units are released concurrently.

Compounds of Formula X are pharmaceutically acceptable salts of prodrug conjugates of Bumetanide and Furosemide allowing release of both compounds in the eye. In one embodiment both compounds are released concurrently.

In alternative embodiments, compounds of Formula X are prodrug conjugates of Bumetanide and Piretanide allowing release of both compounds in the eye. In one embodiment both compounds are released concurrently.

In alternative embodiments, compounds of Formula X are prodrug conjugates of Bumetanide and Ozolinone allowing release of both compounds in the eye. In one embodiment both compounds are released concurrently.

In alternative embodiments, compounds of Formula X are prodrug conjugates of Bumetanide and ethacrynic acid allowing release of both compounds in the eye. In one embodiment both compounds are released concurrently.

In alternative embodiments, compounds of Formula X are prodrug conjugates of Bumetanide allowing release of two units of Bumetanide in the eye. In one embodiment both units are released concurrently.

Compounds of Formula XI are pharmaceutically acceptable salts of prodrug conjugates of Piretanide and Furosemide allowing release of both compounds in the eye. In one embodiment both compounds are released concurrently.

In alternative embodiments, compounds of Formula XI are prodrug conjugates of Piretanide and Bumetanide allowing release of both compounds in the eye. In one embodiment both compounds are released concurrently.

In alternative embodiments, compounds of Formula XI are prodrug conjugates of Piretanide and Ozolinone allowing release of both compounds in the eye. In one embodiment both compounds are released concurrently.

In alternative embodiments, compounds of Formula XI are prodrug conjugates of Piretanide and ethacrynic acid allowing release of both compounds in the eye. In one embodiment both compounds are released concurrently.

In alternative embodiments, compounds of Formula XI are prodrug conjugates of Piretanide allowing release of two units of Piretanide in the eye. In one embodiment both units are released concurrently.

Compounds of Formula XII are pharmaceutically acceptable salts of prodrug conjugates of Ozolinone and Furosemide allowing release of both compounds in the eye. In one embodiment both compounds are released concurrently.

In alternative embodiments, compounds of Formula XII and Formula XII′ are prodrug conjugates of Ozolinone and Bumetanide allowing release of both compounds in the eye. In one embodiment both compounds are released concurrently.

In alternative embodiments, compounds of Formula XII and Formula XII′ are prodrug conjugates of Ozolinone and Piretanide allowing release of both compounds in the eye. In one embodiment both compounds are released concurrently.

In alternative embodiments, compounds of Formula XII and Formula XII′ are prodrug conjugates of Ozolinone and ethacrynic acid allowing release of both compounds in the eye. In one embodiment both compounds are released concurrently.

In alternative embodiments, compounds of Formula XII and Formula XII′ are prodrug conjugates of Ozolinone allowing release of two units of Ozolinone in the eye. In one embodiment both units are released concurrently.

Compounds of Formula XIII are single agent prodrugs of the loop diuretic Furosemide.

In alternative embodiments, compounds of Formula XIII are pharmaceutically acceptable salts of hydrophobic prodrugs of Furosemide.

Compounds of Formula XIV are single agent prodrugs of the loop diuretic Bumetanide.

In alternative embodiments, compounds of Formula XIV are pharmaceutically acceptable salts of hydrophobic prodrugs of Bumetanide.

Compounds of Formula XV are single agent prodrugs of the loop diuretic Piretanide.

In alternative embodiments, compounds of Formula XV are pharmaceutically acceptable salts of hydrophobic prodrugs of Piretanide.

Compounds of Formula XVI and Formula XVI′ are single agent prodrugs of Ozolinone, the active metabolite of the loop diuretic Etozolin.

In alternative embodiments, compounds of Formula XVI and Formula XVI′ are pharmaceutically acceptable salts of hydrophobic prodrugs of Ozolinone, the active metabolite of the loop diuretic Etozolin.

Compounds of Formula XVII are single agent prodrugs of Furosemide.

Compounds of Formula XVII are single agent prodrugs of Bumetanide.

Compounds of Formula XIX are single agent prodrugs of Piretanide.

Compounds of Formula XX and Formula XX′ are single agent prodrugs of Ozolinone, the active metabolite of the loop diuretic Etozolin.

Compounds of Formula XXI are prodrug conjugates of Furosemide and Brimonidine allowing release of both compounds in the eye. In one embodiment both compounds are released concurrently.

In alternative embodiments, compounds of Formula XXI are prodrug conjugates of a carbonic anhydrase inhibitor and Furosemide allowing release of both compounds in the eye. In one embodiment both compounds are released concurrently.

In alternative embodiments, compounds of Formula XXI are prodrug conjugates of a dual leucine zipper kinase inhibitor and Furosemide allowing release of both compounds in the eye. In one embodiment both compounds are released concurrently.

In alternative embodiments, compounds of Formula XXI are single agent prodrug conjugates of a ROCK inhibitor and Furosemide allowing release of both compounds in the eye. In one embodiment both compounds are released concurrently.

In alternative embodiments, compounds of Formula XXI are single agent prodrug conjugates of Timolol and Furosemide allowing release of both compounds in the eye. In one embodiment both compounds are released concurrently.

Compounds of Formula XXII are prodrug conjugates of Bumetanide and Brimonidine allowing release of both compounds in the eye. In one embodiment both compounds are released concurrently.

In alternative embodiments, compounds of Formula XXII are prodrug conjugates of a carbonic anhydrase inhibitor and Bumetanide allowing release of both compounds in the eye. In one embodiment both compounds are released concurrently.

In alternative embodiments, compounds of Formula XXII are prodrug conjugates of a dual leucine zipper kinase inhibitor and Bumetanide allowing release of both compounds in the eye. In one embodiment both compounds are released concurrently.

In alternative embodiments, compounds of Formula XXII are single agent prodrug conjugates of a ROCK inhibitor and Bumetanide allowing release of both compounds in the eye. In one embodiment both compounds are released concurrently.

In alternative embodiments, compounds of Formula XXII are single agent prodrug conjugates of Timolol and Bumetanide allowing release of both compounds in the eye. In one embodiment both compounds are released concurrently.

Compounds of Formula XXIII are prodrug conjugates of Piretanide and Brimonidine allowing release of both compounds in the eye. In one embodiment both compounds are released concurrently.

In alternative embodiments, compounds of Formula XXIII are prodrug conjugates of a carbonic anhydrase inhibitor and Piretanide allowing release of both compounds in the eye. In one embodiment both compounds are released concurrently.

In alternative embodiments, compounds of Formula XXIII are prodrug conjugates of a dual leucine zipper kinase inhibitor and Piretanide allowing release of both compounds in the eye. In one embodiment both compounds are released concurrently.

In alternative embodiments, compounds of Formula XXIII are single agent prodrug conjugates of a ROCK inhibitor and Piretanide allowing release of both compounds in the eye. In one embodiment both compounds are released concurrently.

In alternative embodiments, compounds of Formula XXIII are single agent prodrug conjugates of Timolol and Piretanide allowing release of both compounds in the eye. In one embodiment both compounds are released concurrently.

Compounds of Formula XIV and Formula XIV′ are prodrug conjugates of Ozolinone and Brimonidine allowing release of both compounds in the eye. In one embodiment both compounds are released concurrently.

In alternative embodiments, compounds of Formula XIV and Formula XIV′ are prodrug conjugates of a carbonic anhydrase inhibitor and Ozolinone allowing release of both compounds in the eye. In one embodiment both compounds are released concurrently.

In alternative embodiments, compounds of Formula XIV and Formula XIV′ are prodrug conjugates of a dual leucine zipper kinase inhibitor and Ozolinone allowing release of both compounds in the eye. In one embodiment both compounds are released concurrently.

In alternative embodiments, compounds of Formula XIV and Formula XIV′ are single agent prodrug conjugates of a ROCK inhibitor and Ozolinone allowing release of both compounds in the eye. In one embodiment both compounds are released concurrently.

In alternative embodiments, compounds of Formula XIV and Formula XIV′ are single agent prodrug conjugates of Timolol and Ozolinone allowing release of both compounds in the eye. In one embodiment both compounds are released concurrently.

These compounds can be used to treat an ocular disorder in a host, for example a human, in need thereof. In one embodiment, a method for the treatment of such a disorder is provided that includes the administration of an effective amount of a compound of Formula I, Formula II, Formula III, Formula IV, Formula IV′ Formula V, Formula VI, Formula VII, Formula VIII, Formula VIII′, Formula IX, Formula X, Formula XI, Formula XII, Formula XII′, Formula XIII, Formula XIV, Formula XV, Formula XVI, Formula XVI′, Formula XVII, Formula XVIII, Formula XIX, Formula XX, Formula XX′, Formula XXI, Formula XXII, Formula XXIII, Formula XXIV, or Formula XXIV′, or a pharmaceutically acceptable salt or composition thereof, optionally in a pharmaceutically acceptable carrier, including a polymeric carrier, as described in more detail below.

This invention also includes microparticles for ocular delivery that include an effective amount of a loop diuretic selected from furosemide, bumetanide, piretanide, and etozolin or a combination thereof or a combination with a prodrug described herein wherein the microparticle releases the loop diuretic for at least 1 month, 2 months, 3 months, 4 months, 5 months, or 6 months. In other embodiments, the microparticle for ocular delivery includes an effective amount of a compound selected from Compound 26 or Compound 78, wherein the microparticle releases the active agent for at least 1 month, 2 months, 3 months, 4 months, 5 months, or 6 months.

In one embodiment, the microparticles have a diameter greater than 10 μM and include a core comprising one or more biodegradable polymers and a therapeutic agent selected from furosemide, bumetanide, piretanide, and etozolin. In non-limiting embodiments, the microparticles have a diameter from about 10 μm to 60 μm, from about 20 μm to about 40 μm, or from about 25 μM to about 35 μM. In one non-limiting embodiment, the microparticle comprises furosemide, bumetanide, piretanide, or etozolin encapsulated in a blend of one or more hydrophobic polymers and an amphiphilic polymer. As discussed above, the one or more hydrophobic polymers and amphiphilic polymer are, for example (i) a PLGA polymer or PLA polymer as described herein and (ii) a PLGA-PEG or PLA-PEG copolymer; (i) a PLGA polymer; (ii) a PLA polymer; and, (iii) a copolymer of PLGA-PEG or PLA-PEG; or (i) a PLA polymer; (ii) a PLGA polymer; (iii) a PLGA polymer that has a different ratio of lactide and glycolide monomers than the PLGA in (ii); and, (iv) a PLGA-PEG or PLA-PEG copolymer.

Example 15 provides examples of furosemide and bumetanide microparticles wherein furosemide or bumetanide are encapsulated in 99% PLGA and 1% PLGA-PEG. In one embodiment, the microparticle comprises furosemide or bumetanide encapsulated in PLGA and PLGA-PEG wherein the drug is released over a period of at least 1 month, 2 month, 3 months, 4 months, 5 months, or 6 months. In one embodiment, the microparticle comprises furosemide or bumetanide encapsulated in PLA and PLGA-PEG wherein the drug is released over a period of at least 1 month, 2 month, 3 months, 4 months, 5 months, or 6 months. In one embodiment, the microparticle comprises furosemide or bumetanide encapsulated in PLA, PLGA, and PLGA-PEG wherein the drug is released over a period of at least 1 month, 2 month, 3 months, 4 months, 5 months, or 6 months.

The invention also includes the use of a loop diuretic selected from furosemide, bumetanide, piretanide, and etozolin or a combination thereof of a combination with a prodrug described herein for the treatment of an ocular disorder wherein the loop diuretic is administered via intravitreal, intrastromal, intracameral, sub-tenon, sub-retinal, retro-bulbar, peribulbar, suprachoroidal, choroidal, subchoroidal, conjunctival, episcleral, posterior juxtascleral, circumcorneal, or tear duct injection. In one embodiment, furosemide, bumetanide, or piretanide are administered in a site that is not near the trabecular meshwork. In an alternative embodiment, etozolin is administered via subconjunctival injection.

In one embodiment, the loop diuretic is administered in a dosage form that contains from about 1 μg to 10 mg, from about 1 μg to 1 mg, from about 1 μg to 100 μg, from about 1 μg to 50 g, from about 1 μg to 10 μg, or from about 1 μg to 5 μg.

Another embodiment is provided that includes the administration of an effective amount of an active compound or a pharmaceutically acceptable salt thereof, optionally in a pharmaceutically acceptable carrier, including a polymeric carrier, to a host to treat an ocular or other disorder that can benefit from topical or local delivery. The therapy can be delivery to the anterior or posterior chamber of the eye. In specific aspects, the active compound is administered to treat a disorder of the cornea, conjunctiva, aqueous humor, iris, ciliary body, lens sclera, choroid, retinal pigment epithelium, neural retina, optic nerve or vitreous humor.

Any of the compounds described herein (Formula I, Formula II, Formula III, Formula IV, Formula IV′ Formula V, Formula VI, Formula VII, Formula VIII, Formula VIII′, Formula IX, Formula X, Formula XI, Formula XII, Formula XII′, Formula XIII, Formula XIV, Formula XV, Formula XVI, Formula XVI′, Formula XVII, Formula XVIII, Formula XIX, Formula XX, Formula XX′, Formula XXI, Formula XXII, Formula XXIII, Formula XXIV, or Formula XXIV′) can be administered to the eye in a composition as described further herein in any desired form of administration, including via intravitreal, intrastromal, intracameral, sub-tenon, sub-retinal, retro-bulbar, peribulbar, suprachoroidal, choroidal, subchoroidal, conjunctival, subconjunctival, episcleral, posterior juxtascleral, circumcorneal, and tear duct injections, or through a mucus, mucin, or a mucosal barrier, in an immediate or controlled release fashion. In one embodiment, any of the compounds described herein (Formula I, Formula II, Formula III, Formula IV, Formula IV′ Formula V, Formula VI, Formula VII, Formula VIII, Formula VIII′, Formula IX, Formula X, Formula XI, Formula XII, Formula XII′, Formula XIII, Formula XIV, Formula XV, Formula XVI, Formula XVI′, Formula XVII, Formula XVIII, Formula XIX, Formula XX, Formula XX′, Formula XXI, Formula XXII, Formula XXIII, Formula XXIV, or Formula XXIV′) can be administered to the eye via topical administration.

In any of the Formulas described herein (Formula I, Formula II, Formula III, Formula IV, Formula IV′ Formula V, Formula VI, Formula VII, Formula VIII, Formula VIII′, Formula IX, Formula X, Formula XI, Formula XII, Formula XII′, Formula XIII, Formula XIV, Formula XV, Formula XVI, Formula XVI′, Formula XVII, Formula XVIII, Formula XIX, Formula XX, Formula XX′, Formula XXI, Formula XXII, Formula XXIII, Formula XXIV, or Formula XXIV′) if the stereochemistry of a chiral carbon is not specifically designated in the Formula it is intended that the carbon can be used as an R enantiomer, an S enantiomer, or a mixture of enantiomers including a racemic mixture. In Formula V, Formula VI, Formula VII, or Formula VIII, Timolol has (S)-stereochemistry as used in commercial Timolol maleate ophthalmic solutions, such as Istalol® and Timoptic®. On both U.S. FDA labels, Timolol maleate is described as a single enantiomer ((−)-1-(tert-butylamino)-3-[(4-morpholino-1,2,5-thiadiazol-3-yl)oxy]-2-propanol maleate) that “possesses an asymmetric carbon atom in its structure and is provided as the levo-isomer.” The (S)-enantiomer has CAS No. 26839-75-8 and the (R)-enantiomer has CAS No. 26839-76-9, but only the (S)-enantiomer is described as “Timolol”. Likewise, compounds presented which are or are analogs of commercial products are provided in their approved stereochemistry for regulatory use, unless stated otherwise.

In addition, moieties that have repetitive units of the same or varying monomers, for example including, but not limited to an oligomer of polylactic acid, polylactide-coglycolide, or polypropylene oxide, that have a chiral carbon can be used with the chiral carbons all having the same stereochemistry, random stereochemistry (by either monomer or oligomer), racemic (by either monomer or oligomer) or ordered but different stereochemistry such as a block of S enantiomer units followed by a block of R enantiomer units in each oligomeric unit. In some embodiments lactic acid is used in its naturally occurring S enantiomeric form.

In certain embodiments, the conjugated active drug is delivered in a biodegradable microparticle or nanoparticle that has at least approximately 5, 7.5, 10, 12.5, 15, 20, 25 or 30% or more by weight conjugated active drug. In some embodiments, the biodegradable microparticle degrades over a period of time and in any event provides controlled delivery that lasts at least approximately 2 months, 3 months, 4 months, 5 months or 6 months or more. In some embodiments, the loaded microparticles are administered via subconjunctival or subchoroidal injection.

In certain embodiments, the conjugated active drug is delivered as the pharmaceutically acceptable salt form. Salt forms of a compound will exhibit distinctive solution and solid-state properties compared to their respective free base or free acid form, and for this reason pharmaceutical salts are used in drug formulations to improve aqueous solubility, chemical stability, and physical stability issues. Lipophilic salt forms of compounds, which have enhanced solubility in lipidic vehicles relative to the free acid or free base forms of compounds, are often advantageous in terms of pharmacological properties due in part to their low melting points. Lipophilic salt forms of compounds are used to increase aqueous solubility for oral and parenteral drug delivery, enhance permeation across hydrophobic barriers, and enhance drug loading in lipid-based formulations.

In all of the polymer moieties described in this specification, where the structures are depicted as block copolymers (for example, blocks of “x” followed by blocks of “y”) it is intended that the polymer can alternately be a random or alternating copolymer (for example, “x” and “y”, are either randomly distributed or alternate). Unless stereochemistry is specifically indicated, each individual moiety of each oligomer that has a chiral center can be presented at the chiral carbon in (R) or (S) configuration or a mixture there of, including a racemic mixture.

In most of the Formulas presented herein, the prodrugs are depicted as one or several active moieties covalently bound to or through a described prodrug moiety(ies) with a defined variable range of each of the active moiety and the prodrug moiety, typically through the use of descriptors x, y, or z. As indicated below, these descriptors can independently have numerical ranges provided below, and in most embodiments, are typically within a smaller range, also as provided below. Each variable is independent such that any of the integers of one variable can be used with any of the integers of the other variable, and each combination is considered separately and independently disclosed, and set out below like this only for space considerations.

For example, x and y can independently be any integer between 1 and 30 (1, 2, 3, 4, 5, 6, 7, 8, 9, 10, 11, 12, 13, 14, 15, 16, 17, 18, 19, 20, 21, 22, 23, 24, 25, 26, 27, 28, 29 or 30). In certain embodiments, x or y can independently be 1, 2, 3, 4, 5, 6, 7, 8, 9, 10, 11, or 12 and in certain aspects, 1, 2, 3, 4, 5, or 6. In certain embodiments, x is 1, 2, 3, 4, 5, 6, 7, or 8. In certain embodiments, y is 1, 2, 3, 4, 5, 6, 7, or 8. In certain embodiments, x is 1, 2, 3, 4, 5, or 6. In certain embodiments, y is 1, 2, 3, 4, 5, or 6. In certain embodiments, y is 1, 2, or 3 and x is 1, 2, 3, 4, 5, or 6. In certain embodiments, x is 1, 2, or 3 and y is 1, 2, 3, 4, 5, or 6. In certain embodiments, x is an integer selected from 1, 2, 3, and 4 and y is 1. In certain embodiments, x is an integer selected from 1, 2, 3, and 4 and y is 2. In certain embodiments, x is in integer selected from 1, 2, 3, and 4 and y is 3.

Where x or y is used in connection with the monomeric residue in an oligomer, including for example but not limited to:

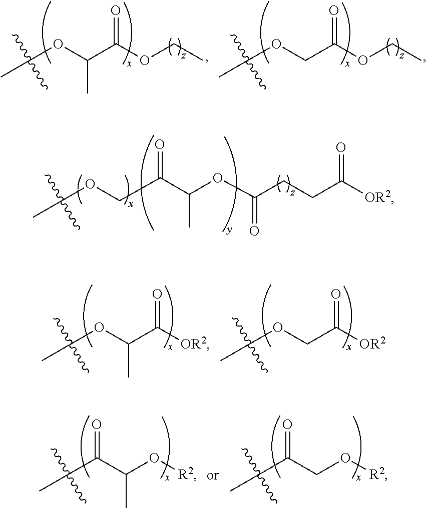

then x or y is in some embodiments independently 1, 2, 3, 4, 5, 6, 7 or 8, and even for example, 2, 4 or 6 residues.

Where z is used in connection with a single atom, such as

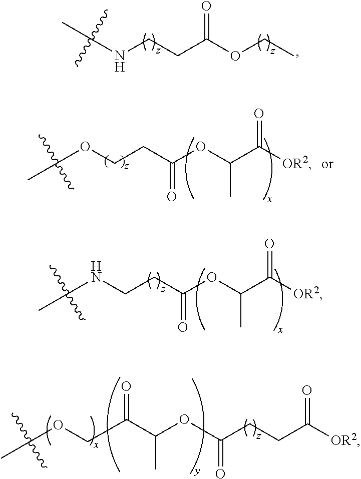

z is independently selected from 0, 1, 2, 3, 4, 5, 6, 7, 8, 9, 10, 11 and 12, and more typically 1, 2, 3, 4, 5 and 6, and even 1, 2, 3 and 4 or 1 and 2.

Various Formulas below use R groups defined in other Formulas, each of which R group is meant to have the definition as presented in the first Formula that it was presented in unless explicitly changed by context.

The disclosure provides a prodrug of Formula I, Formula II, Formula III, Formula IV, and Formula IV′:

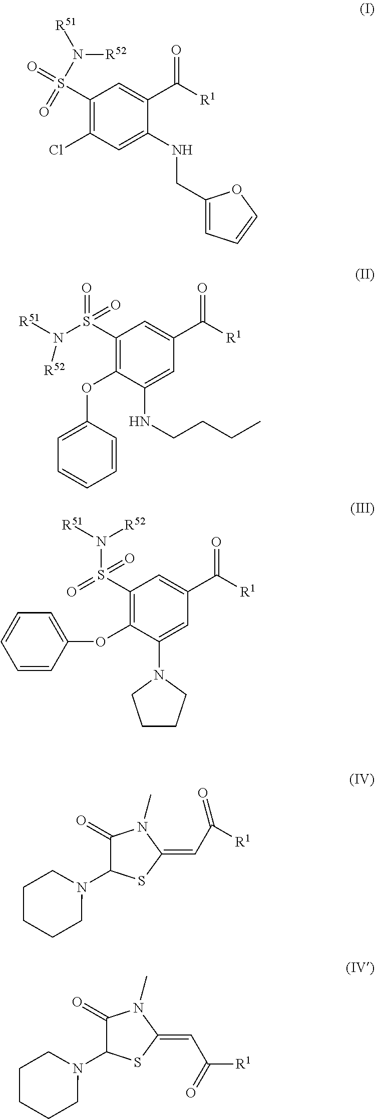

or a pharmaceutically acceptable composition, salt, or isotopic derivative thereof
R¹ is selected from:
(i) —OC₁₅-C₃₀alkylR³, —OC₂-C₃₀alkenylR³, —OC₂-C₃₀alkynylR³, —OC₄-C₃₀alkenylalkynylR³, —OC₁₅-C₃₀alkyl, —OC₂-C₃₀alkenyl, —OC₂-C₃₀alkynyl, and —OC₄-C₃₀alkenylalkynyl;
(ii) —OC₁₅₋₃₀alkyl with at least one R³ substituent on the alkyl chain, —OC₁₋₃₀alkenyl with at least one R³ substituent on the alkenyl chain, and -OC₁₋₃₀alkynyl with at least one R³ substituent on the alkynyl chain;
(iii) —(OCH₂C(O))₁₋₂₀OC₁₋₃₀alkyl, —(OCH(CH₃)C(O))₁₋₂₀OC₁₋₃₀alkyl, —(OCH₂C(O))₁₋₁₀OC₁₋₃₀alkyl, —(OCH(CH₃)C(O))₁₋₁₀OC₁₋₃₀alkyl, —(OCH₂C(O))₄₋₂₀OC₁₋₃₀alkyl, —(OCH(CH₃)C(O))₄₋₂₀OC₁₋₃₀alkyl, —(OCH₂C(O))₁₋₂₀OC₁₋₁₀alkyl, —(OCH(CH₃)C(O))₁₋₂₀OC₁₋₁₀alkyl, —(OCH₂C(O))₁₋₂₀OC₄₋₁₀alkyl, —(OCH(CH₃)C(O))₁₋₂₀OC₄₋₁₀alkyl, —(OCH₂C(O))₁₋₂₀OH, —(OCH(CH₃)C(O))₁₋₂₀OH, —(OCH₂C(O))₁₋₁₀OH, —(OCH(CH₃)C(O))₁₋₁₀OH, —(OCH₂C(O))₄₋₂₀OH, —(OCH(CH₃)C(O))₄₋₂₀OH, —(OCH₂C(O))₄₋₁₀OH, —(OCH(CH₃)C(O))₄₋₁₀OH, —(OCH(CH₃)C(O))₄₋₁₀OC₁₋₁₀alkyl, —(OCH₂C(O))₄₋₁₀OC₁₋₁₀alkyl, —(OCH(CH₃)C(O))₁₋₁₀OC₁₋₁₀alkyl, —(OCH₂C(O))₁₋₁₀OC₁₋₁₀alkyl, —(OCH(CH₃)C(O))₁₋₁₀OC₄₋₁₀alkyl, —(OCH₂C(O))₁₋₁₀OC₄₋₁₀alkyl, —(OCH₂C(O))₁₋₁₀OC₄₋₁₀alkyl, —(OCH(CH₃)C(O))₁₋₁₀OC₄₋₁₀alkyl, —(OCH₂C(O))₁₋₁₀OC₄₋₁₀alkyl, —(OCH(CH₃)C(O))₁₋₁₀OC₄₋₁₀alkyl, —(OCH₂C(O))₁₋₁₀(OCH(CH₃)C(O))₁₋₁₀OC₁₋₃₀alkyl, —(OCH₂C(O))₂₋₁₀(OCH(CH₃)C(O))₂₋₁₀OC₁₋₃₀alkyl, —(OCH₂C(O))₁₋₁₀(OCH(CH₃)C(O))₁₋₁₀OC₁₋₁₂alkyl, —(OCH₂C(O))₁₋₁₀(OCH(CH₃)C(O))₁₋₁₀OC₄₋₂₂alkyl, —(OCH(CH₃)C(O))₁₋₁₀(OCH₂C(O))₁₋₁₀OC₁₋₃₀alkyl, —(OCH(CH₃)C(O))₂₋₁₀(OCH₂C(O))₂₋₁₀OC₁₋₃₀alkyl, —(OCH(CH₃)C(O))₁₋₁₀(OCH₂C(O))₁₋₁₀OC₁₋₁₂alkyl, and —(OCH(CH₃)C(O))₁₋₁₀(OCH₂C(O))₁₋₁₀OC₄₋₂₂alkyl;
(iv) polypropylene glycol, polypropylene oxide, polylactic acid, poly(lactic-co-glycolic acid), polyglycolic acid, a polyester, a polyamide, and other biodegradable polymers, each of which can be capped to complete the terminal valence or to create a terminal ether or ester;

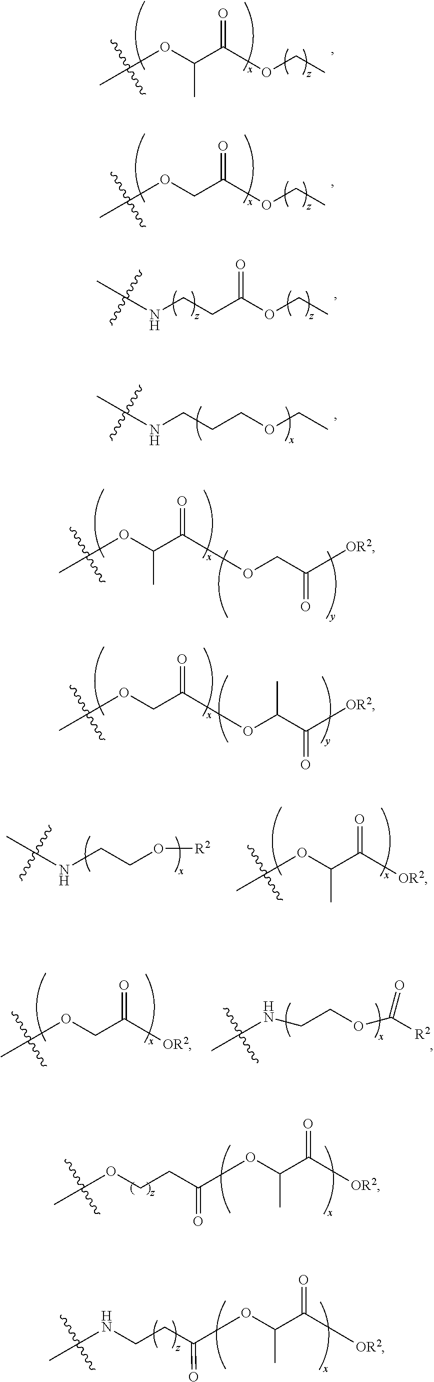

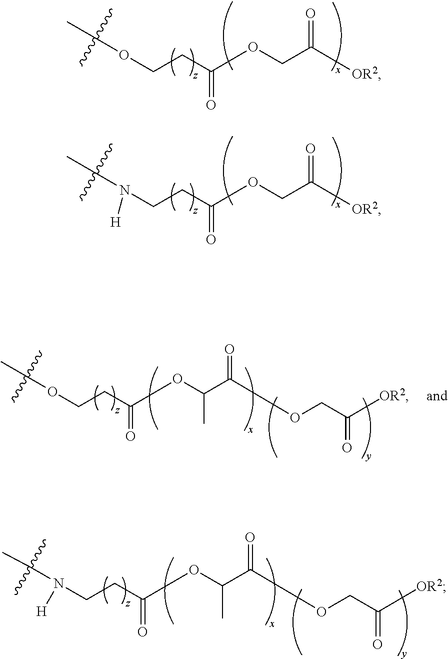

and
(v) —OH; and
(vi) in an alternative embodiment, R¹ is selected from

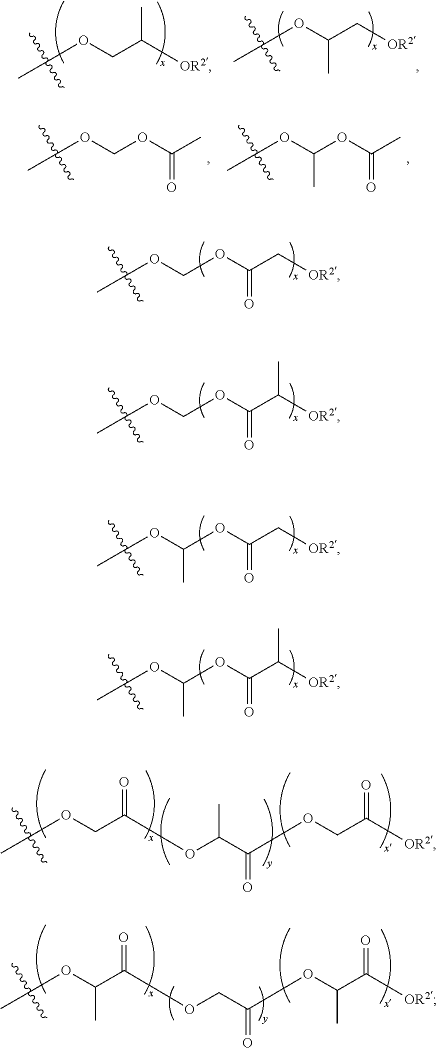

wherein R¹ cannot be OH when R⁵¹ and R⁵² are both hydrogen or when R⁵¹ is hydrogen and R⁵² is C(O)A;

R² is selected at each instance from hydrogen, alkyl, alkenyl, alkynyl cycloalkyl, cycloalkylalkyl, heterocyclyl, heterocycloalkyl, aryl, arylalkyl, heteroaryl, or heteroarylalkyl, each of which except hydrogen may be optionally substituted with R³ if the resulting compound is stable and achieves the desired purpose and wherein the group cannot be substituted with itself, for example alkyl would not be substituted with alkyl;

R^{2′} is selected at each instance from hydrogen and C(O)A;

R³ is selected from halogen, hydroxyl, cyano, mercapto, amino, alkoxy, cycloalkyl, cycloalkylalkyl, heterocyclyl, heterocycloalkyl, aryl, arylalkyl, heteroaryl, heteroarylalkyl, aryloxy, —S(O)₂alkyl, —S(O)alkyl, —P(O)(Oalkyl)₂, B(OH)₂, —Si(CH₃)₃, —COOH, —COOalkyl, and —CONH₂, each of which except halogen, cyano, and —Si(CH₃)₃ may be optionally substituted, for example with halogen, alkyl, aryl, heterocycle or heteroaryl if desired and if the resulting compound is stable and achieves the desired purpose and wherein the group cannot be substituted with itself, for example alkyl would not be substituted with alkyl;

R⁵¹ and R⁵² are independently selected from
(i) hydrogen,

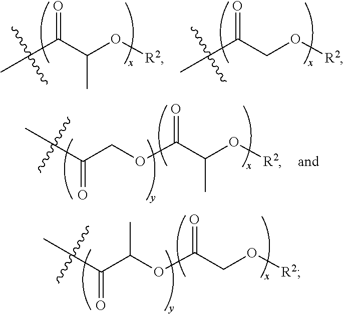

and
(ii) in an alternative embodiment, C(O)A;

x and y at each instance can independently be any integer between 1 and 30 (1, 2, 3, 4, 5, 6, 7, 8, 9, 10, 11, 12, 13, 14, 15, 16, 17, 18, 19, 20, 21, 22, 23, 24, 25, 26, 27, 28, 29 or 30); and

z is independently selected from any integer between 0 and 12 (0, 1, 2, 3, 4, 5, 6, 7, 8, 9, 10, 11, or 12); and

A is selected from H, alkyl, cycloalkyl, cycloalkylalkyl, heterocycle, heterocycloalkyl, aryl, arylalkyl, heteroaryl, heteroarylalkyl, aryloxy, and alkyloxy wherein each group can be optionally substituted with another desired substituent group which is pharmaceutically acceptable and sufficiently stable under the conditions of use, for example selected from R³.

In one embodiment, —C₁-C₃₀ as used in the definition of R¹ is —C₁-C₂₈, —C₁-C₂₆, —C₁-C₂₄, —C₁-C₂₂, —C₁-C₂₀, —C₁-C₁₈, —C₁-C₁₆, —C₁-C₁₄, —C₁-C₁₂, —C₁-C₁₀, —C₁-C₈, —C₁-C₆, —C₁-C₅, or —C₁-C₄.

In one embodiment, —C₁-C₂₀ as used in the definition of R¹ is —C₁-C₁₈, —C₁-C₁₆, —C₁-C₁₄, —C₁-C₁₂, —C₁-C₁, —C₁-C₈, —C₁-C₆, —C₁-C₅, or —C₁-C₄.

In one embodiment, —C₂-C₃₀ as used in the definition of R¹ is —C₂-C₂₈, —C₂-C₂₆, —C₂-C₂₄, —C₂-C₂₂, —C₂-C₂₀, —C₂-C₁₈, —C₂-C₁₆, —C₂-C₁₄, —C₂-C₁₂, —C₂-C₁₀, —C₂-C₈, —C₂-C₆, —C₂-C₅, or —C₂-C₄.

In one embodiment, —C₄-C₂₀ as used in the definition of R¹ is-C₄-C₁₅, —C₄-C₁₆, —C₄-C₁₄, —C₄-C₁₂, —C₄-C₁₀, —C₄-C₅, or —C₄-C₆.

In certain embodiments, x and y are independently selected from 1, 2, 3, 4, 5, 6, 7, 8, 9, and 10.

In certain embodiments, x and y are independently selected from 1, 2, 3, 4, 5, and 6.

In certain embodiments, x and y are independently selected from 1, 2, 3, 4, 5, and 6.

In certain embodiments, x and y are independently selected from 1, 2, 3, and 4.

In certain embodiments, x and y are independently selected from 1, 2, and 3.

In certain embodiments, x is selected from 1, 2, 3, 4, 5, and 6 and y is selected from 1, 2, 3, 4, 5, 6, 7, 8, 9, 10, 11, and 12.

In certain embodiments, y is selected from 1, 2, 3, 4, 5, and 6 and x is selected from 1, 2, 3, 4, 5, 6, 7, 8, 9, 10, 11, and 12.

In certain embodiments, x is selected from 1, 2, 3, 4, 5, and 6 and y is selected from 1, 2, 3, 4, 5, 6, 7, 8, 9, 10, 11, and 12.

In certain embodiments, y is selected from 1, 2, 3, 4, 5, and 6 and x is selected from 1, 2, 3, 4, 5, 6, 7, 8, 9, 10, 11, and 12.

In certain embodiments, x is selected from 1, 2, and 3 and y is selected from 1, 2, 3, 4, 5, and 6.

In certain embodiments, x is selected from 1, 2, 3, 4, 5, and 6, and y is selected from 1, 2, and 3.

In certain embodiments, x is selected from 1, 2, 3, 4, 5, 6, 7, 8, 9, 10, 11, and 12 and z is selected from 1, 2, 3, 4, 5, and 6.

In certain embodiments, x is selected from 1, 2, 3, 4, 5, and 6 and z is selected from 1, 2, and 3.

In certain embodiments, x is 1, 2, or 3 and z is 1.

In certain embodiments, x is 1, 2, or 3 and z is 2.

In certain embodiments, x is 1, 2, or 3 and z is 3.

In one embodiment, R¹ is

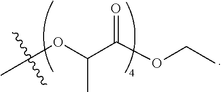

In one embodiment, R¹ is

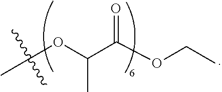

In one embodiment, R¹ is

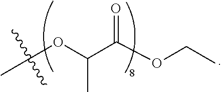

In one embodiment, R¹ is

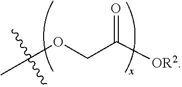

In one embodiment, R¹ is

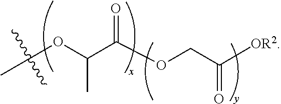

In one embodiment, R¹ is

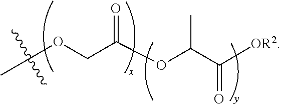

In one embodiment, R¹ is

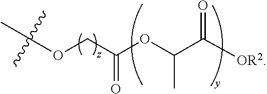

In one embodiment, R¹ is —(OCH(CH₃)C(O))₄₋₂₀OCH₂CH₃.

In one embodiment, R¹ is —(OCH(CH₃)C(O))₄₋₂₀O(CH₂)₁₁CH₃.

In one embodiment, R¹ is —(OCH(CH₃)C(O))₄₋₂₀O(CH₂)₁₇CH₃.

In one embodiment, R¹ is —(OCH(CH₃)C(O))₄OCH₂CH₃.

In one embodiment, R¹ is —(OCH(CH₃)C(O))₄O(CH₂)₁₁CH₃.

In one embodiment, R¹ is —(OCH(CH₃)C(O))₄OCH₂)₁₇CH₃.

In one embodiment, R¹ is —(OCH(CH₃)C(O))₆COCH₂CH₃.

In one embodiment, R¹ is —(OCH(CH₃)C(O))₆O(CH₂)₁₁CH₃.

In one embodiment, R¹ is —(OCH(CH₃)C(O))₆O(CH₂)₁₇CH₃.

In one embodiment, R¹ is —(OCH(CH₃)C(O))₈OOCH₂CH₃.

In one embodiment, R¹ is —(OCH(CH₃)C(O))₈O(CH₂)₁₁CH₃.

In one embodiment, R¹ is —(OCH(CH₃)C(O))₈O(CH₂)₁₇CH₃.

In one embodiment, R¹ is —(OCH₂C(O))(OCH(CH₃)C(O))₄₋₂₀OCH₂CH₃.

In one embodiment, R¹ is —(OCH₂C(O))₂(OCH(CH₃)C(O))₄₋₂₀OCH₂CH₃.

In one embodiment, R¹ is —(OCH₂C(O))(OCH(CH₃)C(O))₄₋₁₀OCH₂CH₃.

In one embodiment, R¹ is —(OCH₂C(O))₂(OCH(CH₃)C(O))₄₋₁₀OCH₂CH₃.

In one embodiment, R¹ is —(OCH₂C(O))(OCH(CH₃)C(O))₆OCH₂CH₃.

In one embodiment, R¹ is —(OCH₂C(O))₂(OCH(CH₃)C(O))₆OCH₂CH₃.

In one embodiment, R¹ is —(OCH(CH₃)C(O))₄₋₂₀O(CH₂)₉₋₁₇CH₃.

In one embodiment, R¹ is —(OCH(CH₃)C(O))₄₋₂₀O(CH₂)₁₁₋₁₇CH₃.

In one embodiment, R¹ is —(OCH(CH₃)C(O))₄₋₂₀O(CH₂)₁₃₋₁₇CH₃.

In one embodiment, R¹ is —(OCH(CH₃)C(O))₄₋₂₀O(CH₂)₁₅₋₁₇CH₃.

In one embodiment, R is —(OCH(CH₃)C(O))₄₋₂₀O(CH₂)₁₁CH₃.

In one embodiment, R¹ is —(OCH(CH₃)C(O))₄₋₂₀O(CH₂)₁₇CH₃.

In one embodiment, R¹ is —(OCH₂C(O))₁₋₂(OCH(CH₃)C(O))₄₋₂₀OCH₂CH₃.

In one embodiment, R¹ is —(OCH₂C(O))₁₋₂(OCH(CH₃)C(O))₄₋₂₀O(CH₂)₁₁CH₃.

In one embodiment, R¹ is —(OCH₂C(O))₁₂(OCH(CH₃)C(O))₄₋₂₀O(CH₂)₁₇CH₃.

In one embodiment, R¹ is —(OCH₂C(O))₁₋₂(OCH(CH₃)C(O))₄₋₂₀O(CH₂)₉₋₁₇CH₃.

In one embodiment, R¹ is —(OCH₂C(O))₁₋₂(OCH(CH₃)C(O))₄₋₂₀O(CH₂)₁₁₋₁₇CH₃.

In one embodiment, R¹ is —(OCH₂C(O))₁₋₂(OCH(CH₃)C(O))₄₋₂₀O(CH₂)₁₃₋₁₇CH₃.

In one embodiment, R¹ is —(OCH₂C(O))₁₋₂(OCH(CH₃)C(O))₄₋₂₀O(CH₂)₁₅₋₁₇CH₃.

In an alternative embodiment, R¹ is

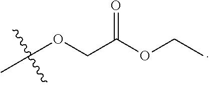 [SMILES: *OCC(=O)OCC]

In an alternative embodiment, R¹ is

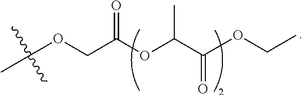

In an alternative embodiment, R¹ is

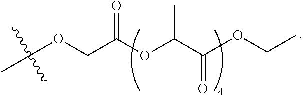

In an alternative embodiment, R¹ is

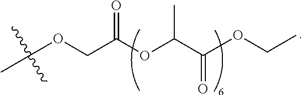

In an alternative embodiment, R¹ is selected from

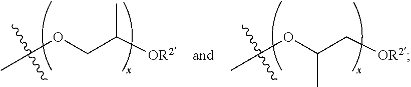

In one embodiment, C₁₋₃₀alkyl as used in the definition of R¹ is C₁₋₂₈, C₁₋₂₆, C₁₋₂₄, C₁₋₂₂, C₁₋₂₀, C₁₋₁₈, C₁₋₁₆, C₁₋₁₄, C₁₋₁₂, C₁₋₁₀, C₁₋₈, C₁₋₆, or C₁₋₄.

In one embodiment, x and y are independently an integer between 1 and 12 (1, 2, 3, 4, 5, 6, 7, 8, 9, 10, 11, or 12). In one embodiment, x and y are independently an integer between 1 and 10 (1, 2, 3, 4, 5, 6, 7, 8, 9, or 10). In one embodiment, x and y are independently an integer between 4 and 10 (4, 5, 6, 7, 8, 9, or 10).

The disclosure also provides a prodrug of Formula V, Formula VI, Formula VII, Formula VIII, and Formula VIII′:

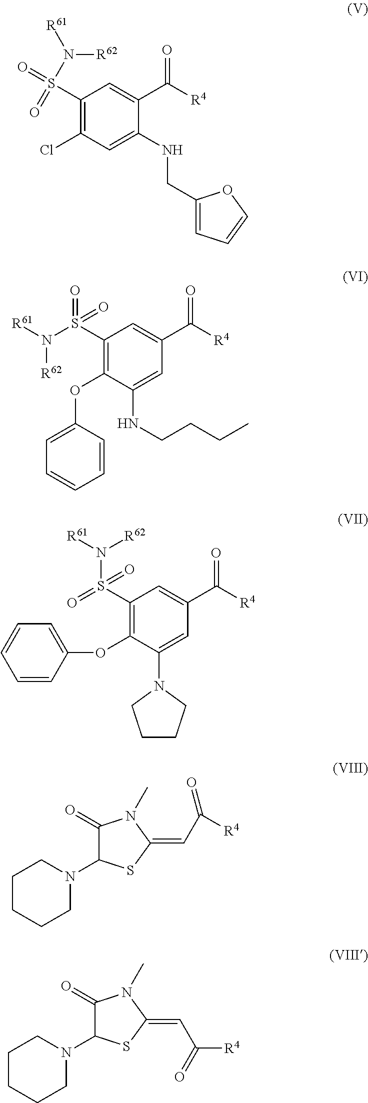

or a pharmaceutically acceptable composition, salt, or isotopic derivative thereof
R⁴ is selected from:

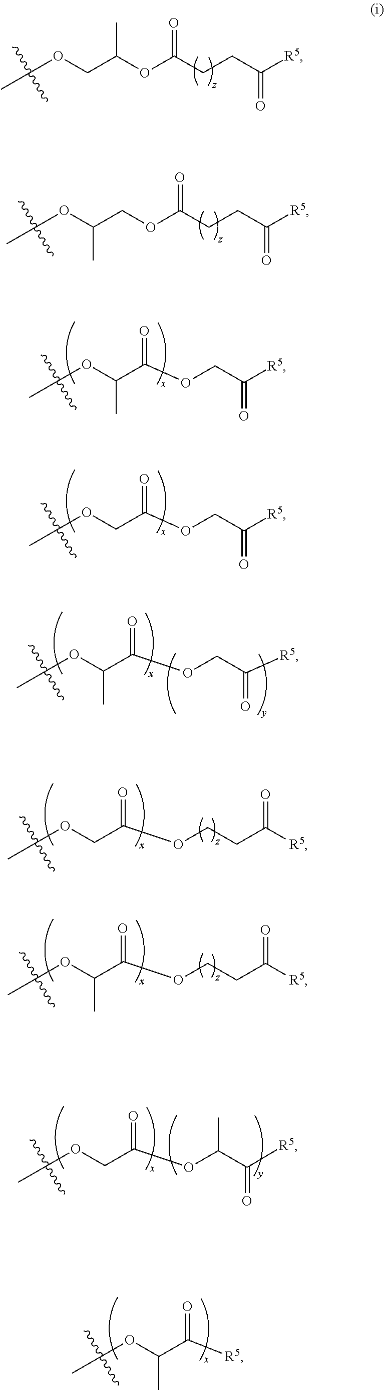

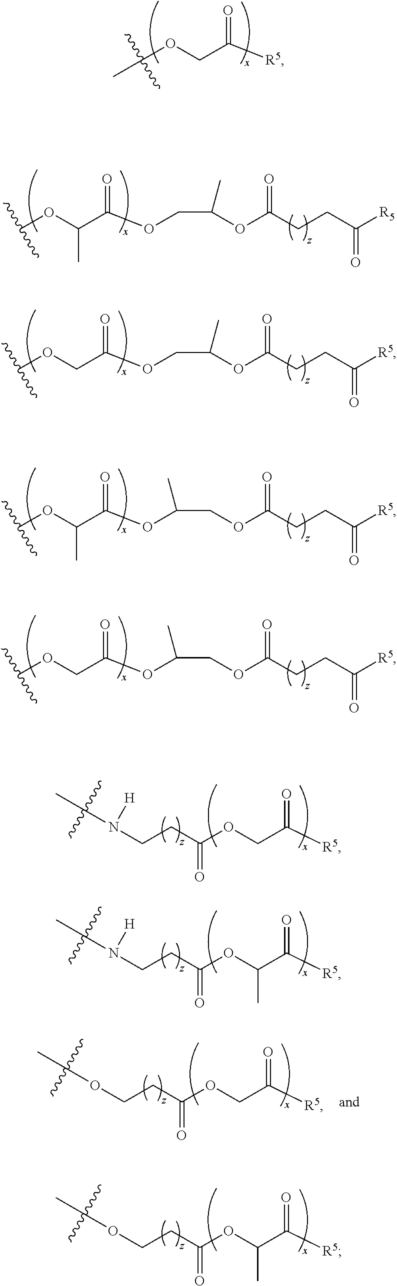

and
(ii) —OH;
(iii) in an alternative embodiment, R⁵; and
(iv) in an alternative embodiment,

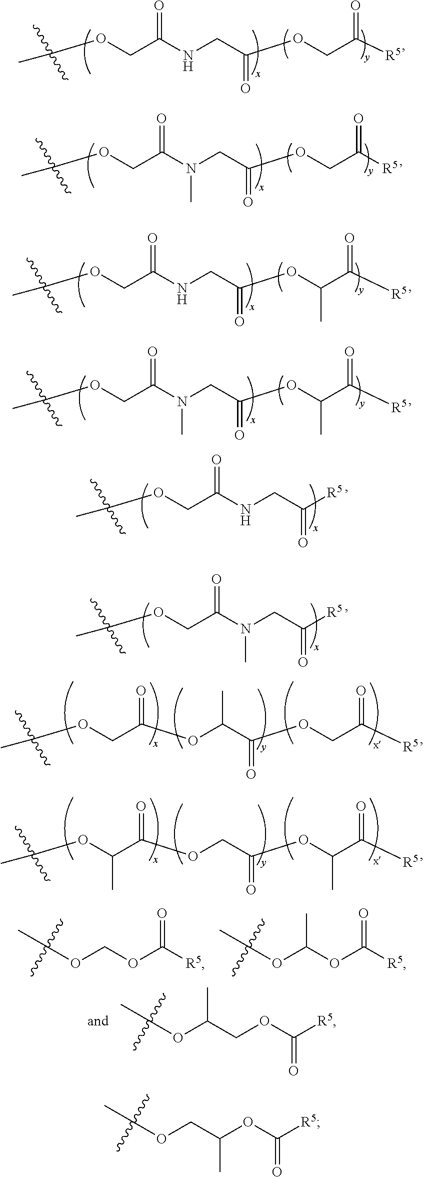

wherein R⁴ cannot be —OH when R⁶¹ and R⁶² are both hydrogen or when R⁶¹ is hydrogen and R⁶² is C(O)A;

R⁵ is independently selected from

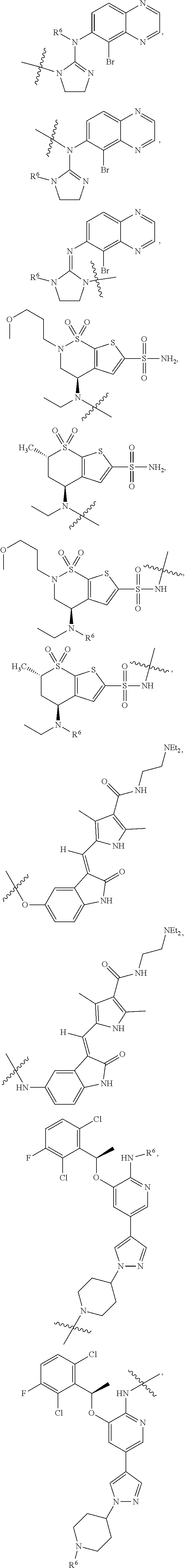

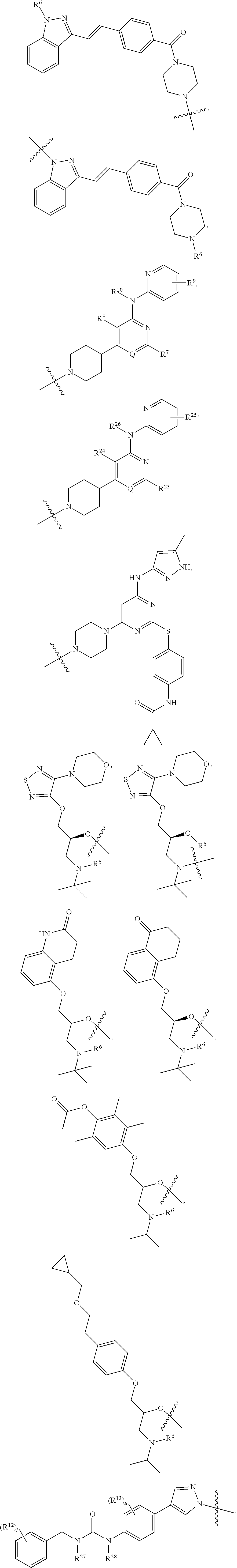

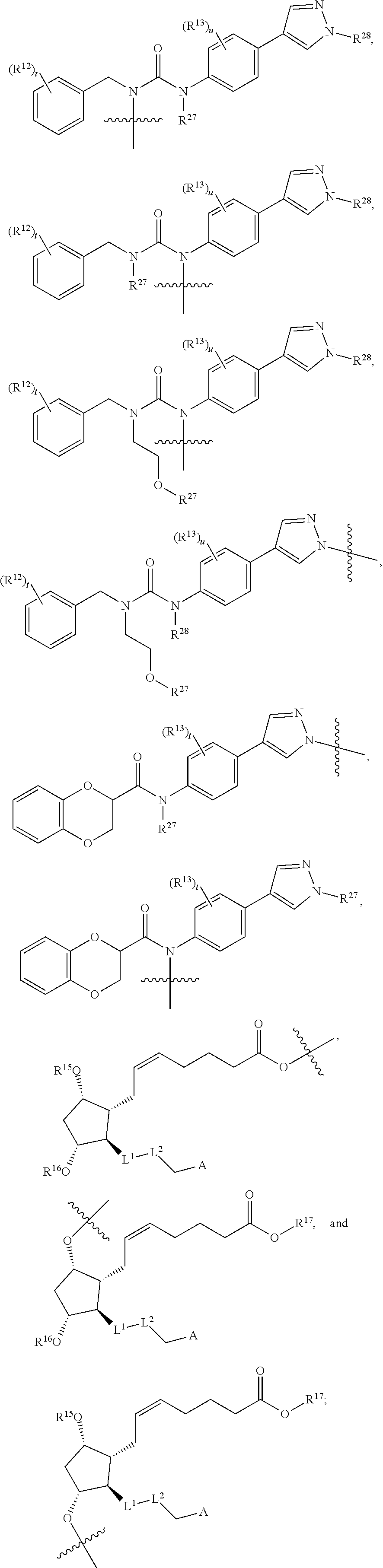

R⁶ is independently selected at each occurrence from

(i) C(O)A, hydrogen,

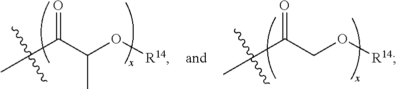

and
(ii) in an alternative embodiment,

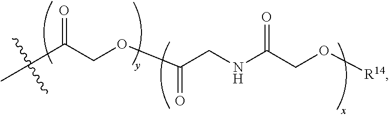

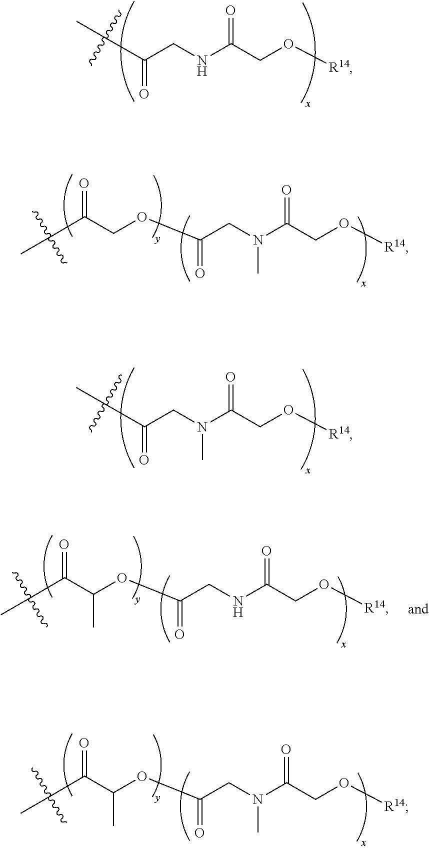

R⁷, R⁸, and R⁹ are independently selected from: hydrogen, halogen, hydroxyl, cyano, mercapto, nitro, amino, aryl, alkyl, alkoxy, alkenyl, alkynyl cycloalkyl, cycloalkylalkyl, heterocyclyl, heterocycloalkyl, aryl, arylalkyl, heteroaryl, heteroarylalkyl, aryloxy, —S(O)₂alkyl, —S(O)alkyl, —P(O)(Oalkyl)₂, B(OH)₂, —Si(CH₃)₃, —COOH, —COOalkyl, —CONH₂,

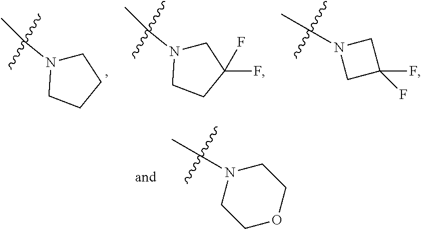 [SMILES: *N1CCCC1 ; *N1CCC(F)(F)C1 ; *N1CC(F)(F)C1 ; *N1CCOCC1]

each of which except halogen, nitro, and cyano, may be optionally substituted, for example with halogen, alkyl, aryl, heterocycle or heteroaryl;

R¹⁰ is selected from H, C(O)A, —C₀-C₁₀alkylR³, —C₂-C₁₀alkenylR³, —C₂-C₁₀alkynylR³, —C₂-C₁₀alkenyl, and —C₂-C₁₀alkynyl;

R¹¹ and R^{11′} are independently selected from —C(O)R¹⁸, —C(O)A, and hydrogen;

R¹² is selected from hydrogen, —C(O)NR¹¹R^{11′}, —C(O)R¹¹, —C(O)OR¹¹, nitro, amino, —NR¹⁹R²⁰, alkyl, alkoxy, alkylalkoxy, alkoxyalkoxy, haloalkoxy, cycloalkyl, heterocycloalkyl, heteroaryl, aryl, and halogen;

R¹³ is selected from hydrogen, —C(O)NR¹¹R^{11′}, —C(O)R¹¹, —C(O)OR¹¹, nitro, amino, —NR¹⁹R²⁰, alkyl, alkoxy, alkylalkoxy, alkoxyalkoxy, haloalkoxy, cycloalkyl, heterocycloalkyl, heteroaryl, aryl, halogen, —O(CH₂)₂NR²¹R²², and —N(CH₃)(CH₂)₂NR²¹R²²;

R¹⁴ is selected from hydrogen, —C(O)A, —C(O)alkyl, aryl, alkyl, cycloalkyl, cycloalkylalkyl, heterocyclyl, heterocycloalkyl, arylalkyl, heteroaryl, and heteroarylalkyl;

R¹⁵ and R¹⁶ are independently selected from: —C(O)R¹⁸, C(O)A, and hydrogen, each of which except hydrogen can be optionally substituted with R³;

R¹⁷ is selected from:
(i) polyethylene glycol, polypropylene glycol, polypropylene oxide, polylactic acid, and poly(lactic-co-glycolic acid), polyglycolic acid, or a polyester, a polyamide, or other biodegradable polymers, wherein a terminal hydroxy or carboxy group can be substituted to create an ether or ester, respectively;
(ii) —C₁₀-C₃₀alkylR³, —C₁₀-C₃₀alkenylR³, —C₁₀-C₃₀alkynylR³, —C₁₀-C₃₀alkenylalkynylR³, —C₁₀-C₃₀alkyl, —C₁₀-C₃₀alkenyl, —C₁₀-C₃₀alkynyl, —C₁₀-C₃₀alkenylalkynyl;
(iii) an unsaturated fatty acid residue including but not limited to the carbon fragment taken from linoleic acid (—(CH₂)₈(CH)₂CH₂(CH)₂(CH₂)₄CH₃)), docosahexaenoic acid (—(CH₂)₃(CHCHCH₂)₆CH₃)), eicosapentaenoic acid (—(CH₂)₄(CHCHCH₂)₅CH₃)), alpha-linolenic acid (—(CH₂)₈(CHCHCH₂)₃CH₃)) stearidonic acid, y-linolenic acid, arachidonic acid, docosatetraenoic acid, palmitoleic acid, vaccenic acid, paullinic acid, oleic acid, elaidic acid, gondoic acid, euric acid, nervonic acid or mead acid; and
(iv) alkyl, cycloalkyl, cycloalkylalkyl, heterocycle, heterocycloalkyl, arylalkyl, heteroarylalkyl;

R¹⁸ is selected from:
(i) —C₁₀-C₃₀alkylR³, —C₁₀-C₃₀alkenylR³, —C₁₀-C₃₀alkynylR³, —C₁₀-C₃₀alkenylalkynylR³, —C₁₀-C₃₀alkyl, —C₁₀-C₃₀alkenyl, —C₁₀-C₃₀alkynyl, —C₁₀-C₃₀alkenylalkynyl; and
(ii) an unsaturated fatty acid residue including but not limited to the carbon chains from linoleic acid (—(CH₂)₈(CH)₂CH₂(CH)₂(CH₂)₄CH₃)), docosahexaenoic acid (—(CH₂)₃(CHCHCH₂)₆CH₃)), eicosapentaenoic acid (—(CH₂)₄(CHCHCH₂)₅CH₃)), alpha-linolenic acid (—(CH₂)₈(CHCHCH₂)₃CH₃)), stearidonic acid, y-linolenic acid, arachidonic acid, docosatetraenoic acid, palmitoleic acid, vaccenic acid, paullinic acid, oleic acid, elaidic acid, gondoic acid, euric acid, nervonic acid and mead acid, and wherein, if desired, each of which can be substituted with R³;

R¹⁹ and R²⁰ are independently selected from H, alkyl, —SO₂CH₃, —C(O)CH₃, and —C(O)NH₂;

R²¹ and R²² are independently selected from H, alkyl, —SO₂CH₃, —C(O)CH₃, and —C(O)NH₂;

or R²¹ and R²² can together form a heterocycloalkyl;

R²³, R²⁴, and R²⁵ are independently selected from: hydrogen, halogen, hydroxyl, cyano, mercapto, nitro, amino, aryl, alkyl, alkoxy, alkenyl, alkynyl cycloalkyl, cycloalkylalkyl, heterocyclyl, heterocycloalkyl, aryl, arylalkyl, heteroaryl, heteroarylalkyl, aryloxy, —S(O)₂alkyl, —S(O)alkyl, —P(O)(Oalkyl)₂, B(OH)₂, —Si(CH₃)₃, —COOH, —COOalkyl, —CONH₂,

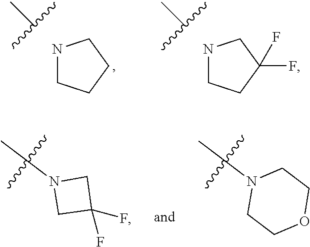

each of which except halogen, nitro, and cyano, may be optionally substituted, for example with halogen, alkyl, aryl, heterocycle or heteroaryl;

R²⁶ is selected from H, C(O)A, —C₀-C₁₀alkylR³, —C₂-C₁₀alkenylR³, —C₂-C₁₀alkynylR³, —C₂-C₁₀alkenyl, and —C₂-C₁₀alkynyl;

R²⁷ and R²⁸ are independently selected from H, C₁-C₃₀alkyl, —C(O)C₁-C₃₀alkyl, C₁-C₃₀heteroalkyl, and C₂-C₃₀alkenyl;

R⁶¹ and R⁶² are independently selected from
(i) hydrogen,

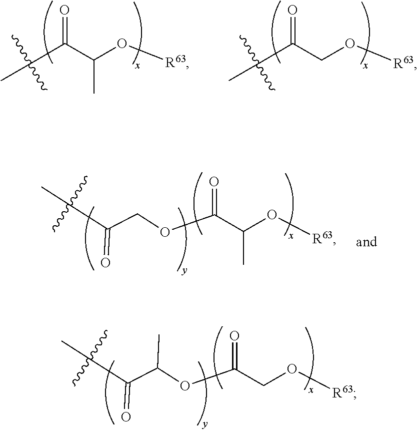

and
(ii) in an alternative embodiment, C(O)A;

R⁶³ is selected from
(i) R²;
(ii) in an alternative embodiment, L³-R⁵;

L¹ is selected from:

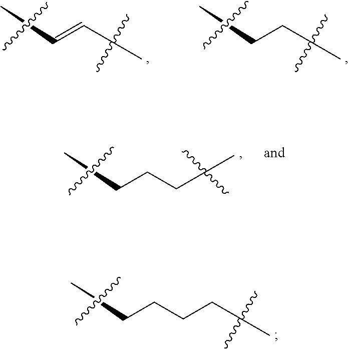

L² is selected from:

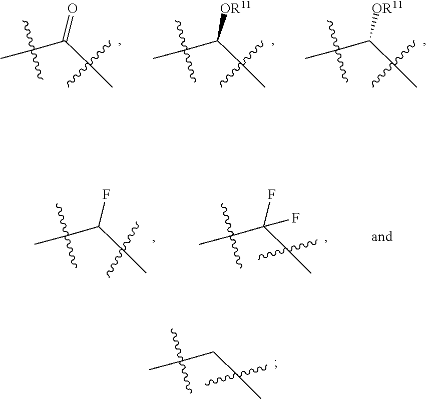

L³ is selected from alkyl, —C(O)—, —C(S), alkyl-C(O)—, and —C(O)-alkyl;

A is selected from H, alkyl, cycloalkyl, cycloalkylalkyl, heterocycle, heterocycloalkyl, aryl, arylalkyl, heteroaryl, heteroarylalkyl, aryloxy, and alkyloxy wherein each group can be optionally substituted with another desired substituent group which is pharmaceutically acceptable and sufficiently stable under the conditions of use, for example selected from R³;

Q is selected from: N, CH, and CR²³;

t and u are independently selected from 0, 1, 2, 3, and 4;

x′ is any integer between 1 and 30 (1, 2, 3, 4, 5, 6, 7, 8, 9, 10, 11, 12, 13, 14, 15, 16, 17, 18, 19, 20, 21, 22, 23, 24, 25, 26, 27, 28, 29 or 30); and

R³, x, y, and z are defined herein.

In certain embodiments, R⁴ is selected from

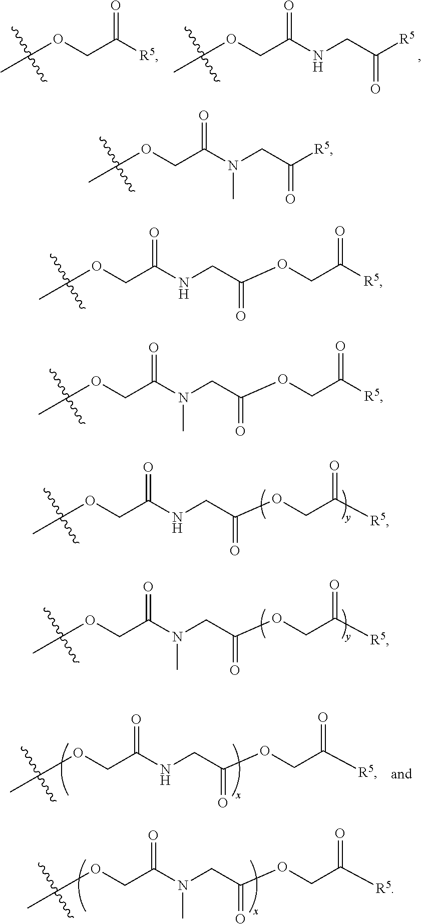

In certain embodiments, R⁶ is selected from

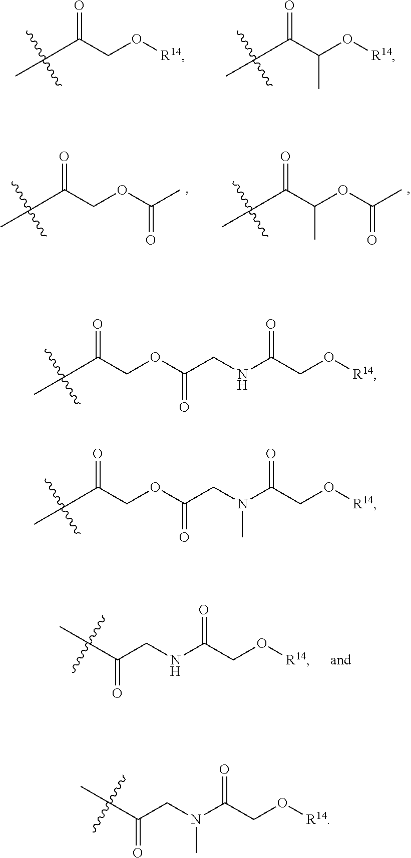

In one embodiment, R⁴ is

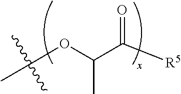

### and R⁵ is

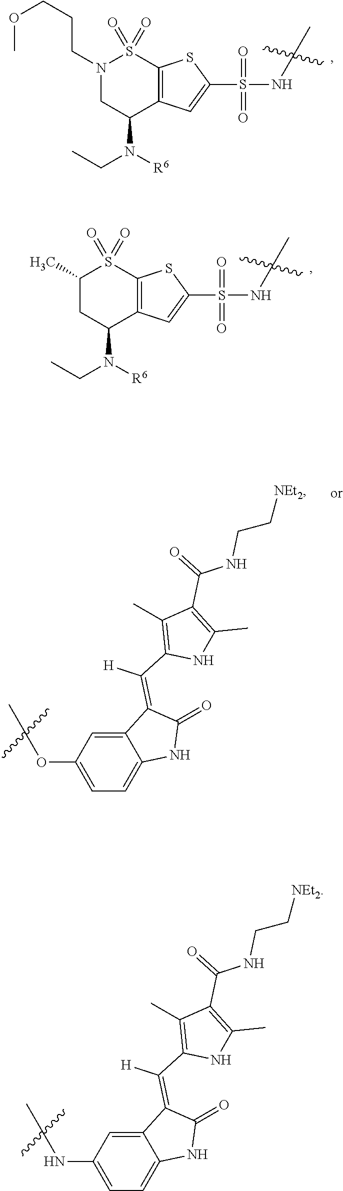

In one embodiment, R⁴ is

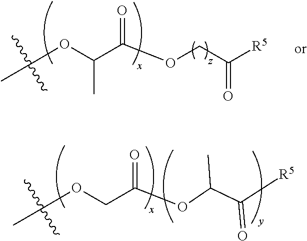

### and R⁵ is

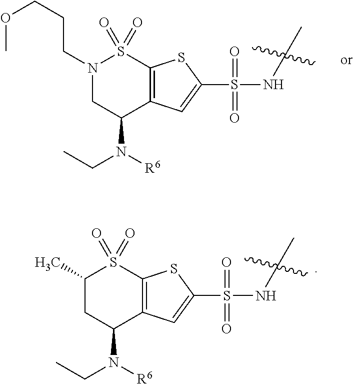

In one embodiment, R⁴ is

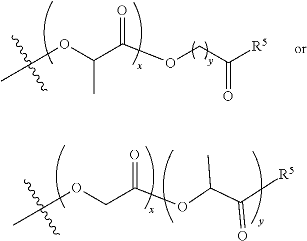

### and R⁵ is

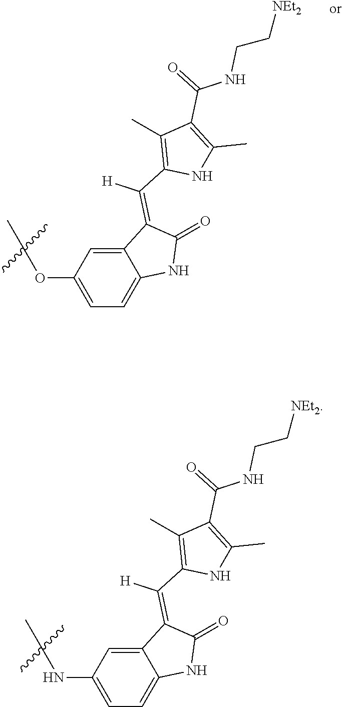

In one embodiment, R⁴ is selected from

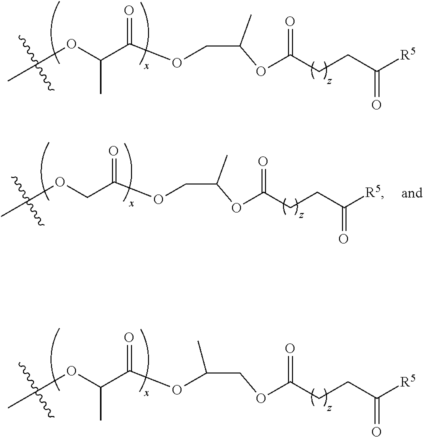

### and R⁵ is

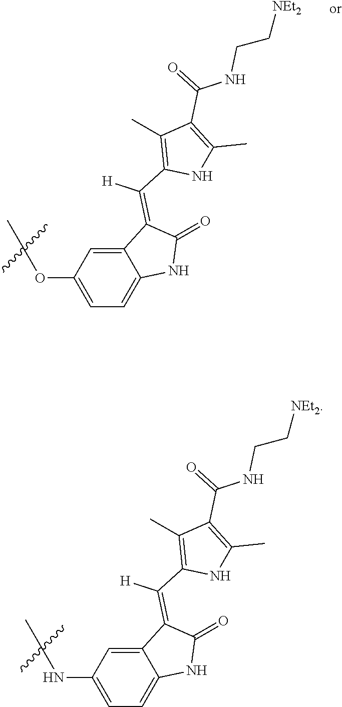

In an alternative embodiment, R⁴ is

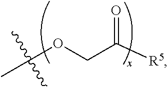

### R⁵ is

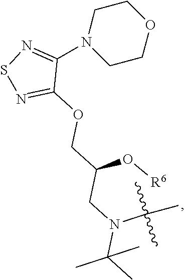

### and R⁶ is

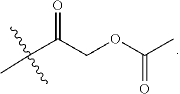 [SMILES: *C(=O)COC(C)=O]

In an alternative embodiment, R⁴ is

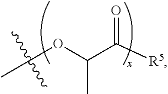

### R⁵ is

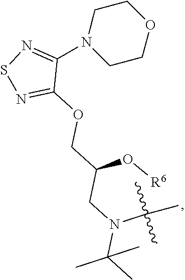

### and R⁶ is

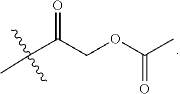 [SMILES: *C(=O)COC(C)=O]

In an alternative embodiment, R⁴ is

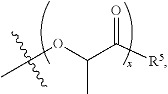

### R⁵ is

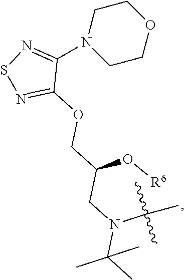

### and R⁶ is

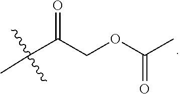 [SMILES: *C(=O)COC(C)=O]

In an alternative embodiment, R⁴ is

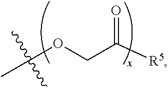

### R⁵ is

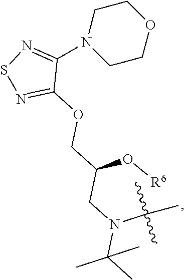

### and R⁶ is

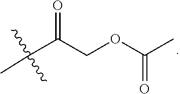 [SMILES: *C(=O)COC(C)=O]

In an alternative embodiment, R⁶ is

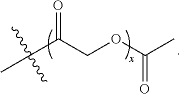

In an alternative embodiment, R⁶ is

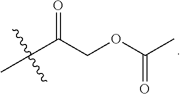 [SMILES: *C(=O)COC(C)=O]

In an alternative embodiment, R⁶ is

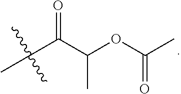 [SMILES: *C(=O)C(C)OC(C)=O]

In an alternative embodiment, R⁵ is selected from

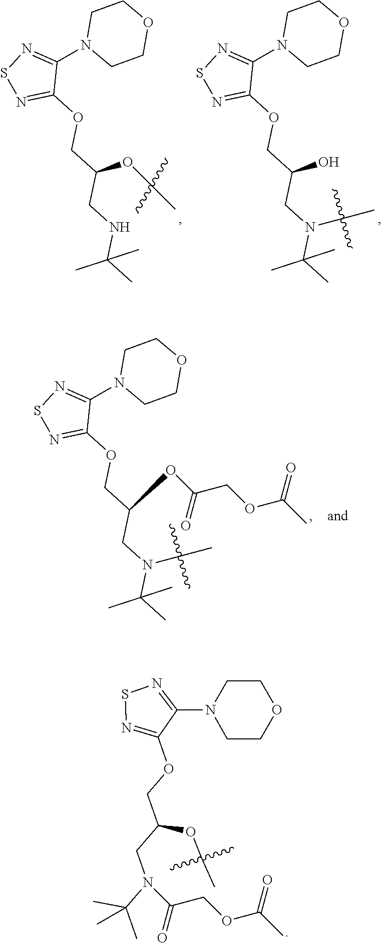

In an alternative embodiment, R⁵ is selected from

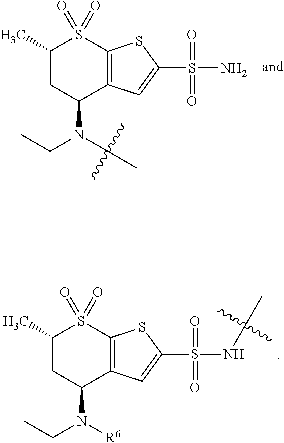

In an alternative embodiment, R⁴ is selected from

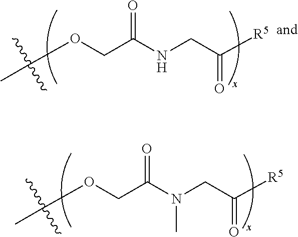

and R⁵ is selected from

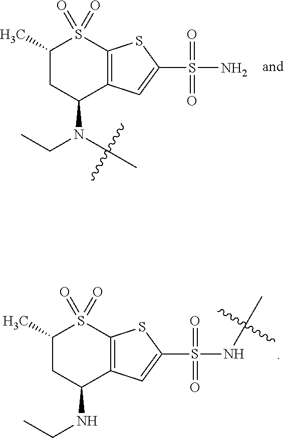 [SMILES: *N(CC)[C@H]1C[C@H](C)S(=O)(=O)c2sc(S(N)(=O)=O)cc21 ; *NS(=O)(=O)c1cc2c(s1)S(=O)(=O)[C@@H](C)C[C@@H]2NCC]

In an alternative embodiment, R⁴ is selected from

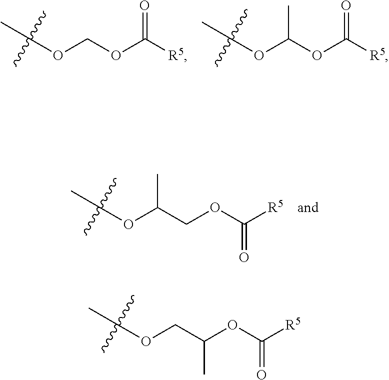

and R⁵ is selected from

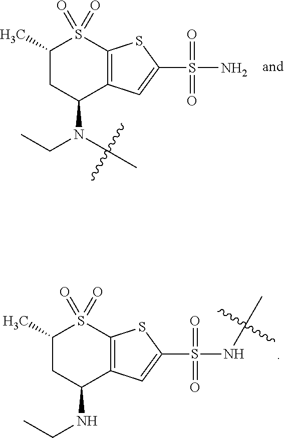 [SMILES: *N(CC)[C@H]1C[C@H](C)S(=O)(=O)c2sc(S(N)(=O)=O)cc21 ; *NS(=O)(=O)c1cc2c(s1)S(=O)(=O)[C@@H](C)C[C@@H]2NCC]

In an alternative embodiment, R⁴ is selected from

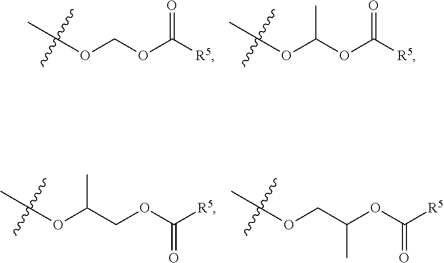

and R⁵ is selected from

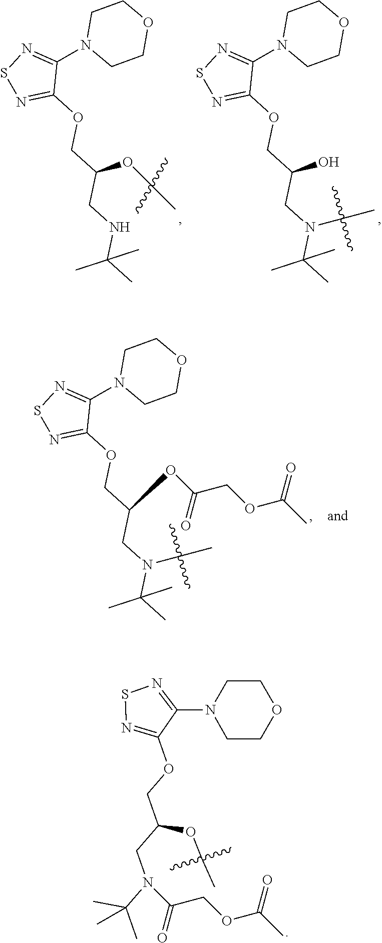

In alternative embodiments, R⁴ is

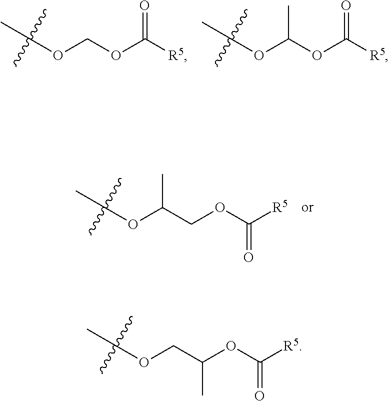

In certain embodiments, x and y are independently selected from 1, 2, 3, 4, 5, and 6.

In certain embodiments, x and y are independently selected from 1, 2, 3, and 4.

In certain embodiments, x and y are independently selected from 1, 2, and 3.

In certain embodiments, x is selected from 1, 2, 3, 4, 5, and 6 and y is selected from 1, 2, 3, 4, 5, 6, 7, 8, 9, 10, 11, and 12.

In certain embodiments, y is selected from 1, 2, 3, 4, 5, and 6 and x is selected from 1, 2, 3, 4, 5, 6, 7, 8, 9, 10, 11, and 12.

In certain embodiments, x is selected from 1, 2, and 3 and y is selected from 1, 2, 3, 4, 5, and 6.

In certain embodiments, x is selected from 1, 2, 3, 4, 5, and 6, and y is selected from 1, 2, and 3.

In certain embodiments, x is selected from 1, 2, 3, 4, 5, 6, 7, 8, 9, 10, 11, and 12 and z is selected from 1, 2, 3, 4, 5, and 6.

In certain embodiments, x is selected from 1, 2, 3, 4, 5, and 6 and z is selected from 1, 2, and 3.

In certain embodiments, x is 1, 2, or 3 and z is 1.

In certain embodiments, x is 1, 2, or 3 and z is 2.

In certain embodiments, x is 1, 2, or 3 and z is 3.

The disclosure also provides a prodrug of Formula IX, Formula X, Formula XI, Formula XII, and Formula XII′:

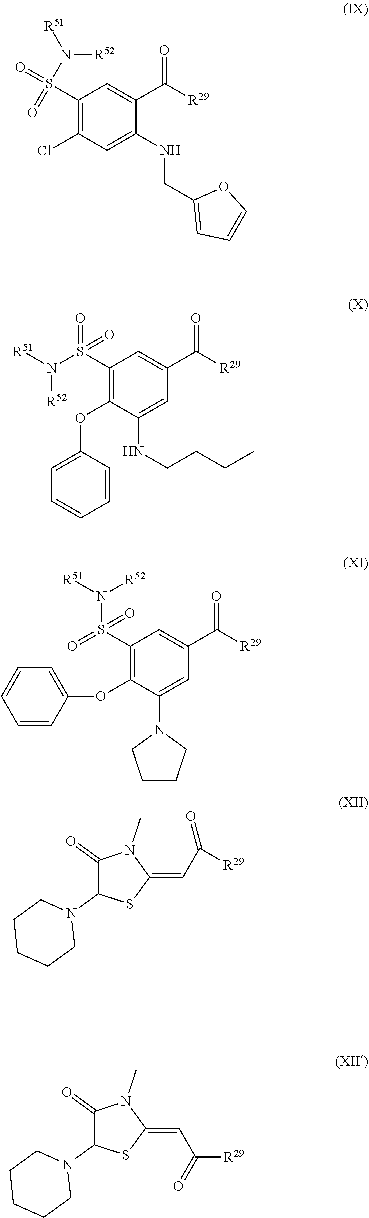

or a pharmaceutically acceptable composition, salt, or isotopic derivative thereof

wherein:

R²⁹ is selected from:

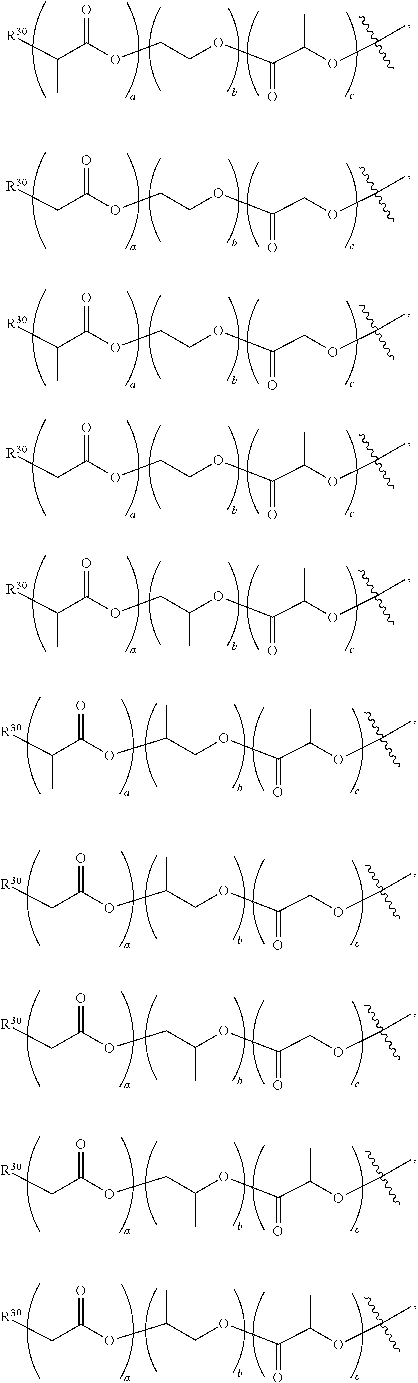

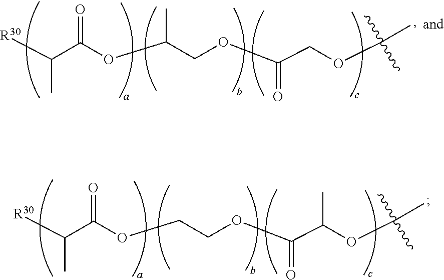

(ii) in an alternative embodiment

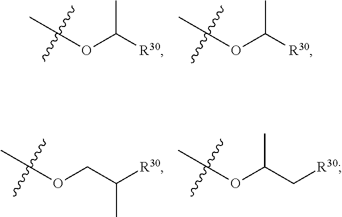

R³⁰ is selected from

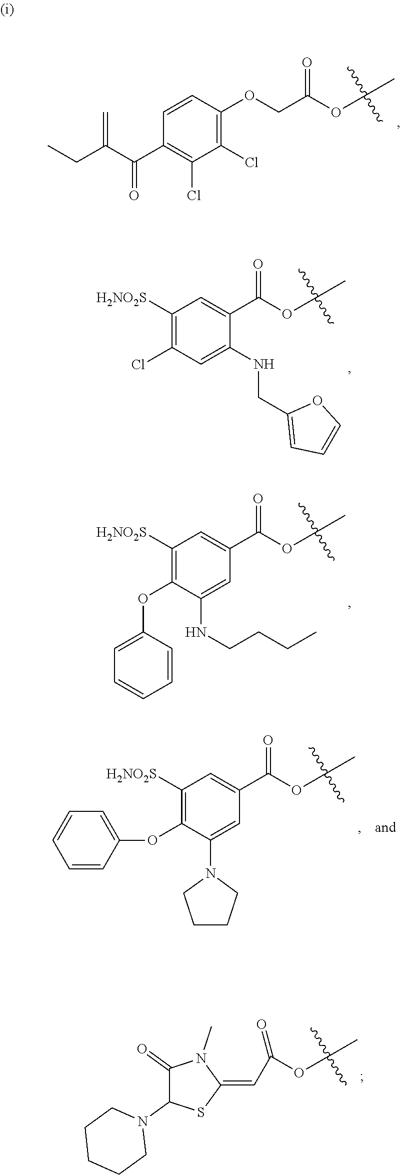 [SMILES: *OC(=O)COc1ccc(C(=O)C(=C)CC)c(Cl)c1Cl ; *OC(=O)c1cc(S(N)(=O)=O)c(Cl)cc1NCc1ccco1 ; *OC(=O)c1cc(NCCCC)c(Oc2ccccc2)c(S(N)(=O)=O)c1 ; *OC(=O)c1cc(N2CCCC2)c(Oc2ccccc2)c(S(N)(=O)=O)c1 ; *OC(=O)/C=C1/SC(N2CCCCC2)C(=O)N1C]

and

(ii) in an alternative embodiment,

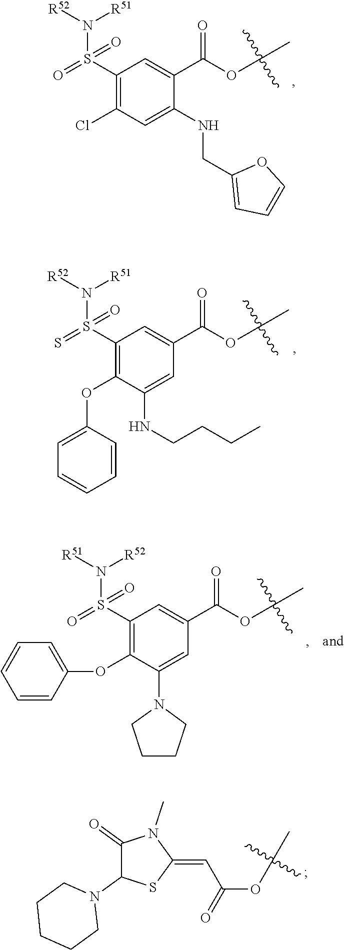

a, b, and c are independently an integer selected from 0 to 30 (0, 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30) wherein a and c cannot both be 0; and

wherein R⁵¹ and R⁵² are as defined herein.

The polymer moieties described in Formula IX, Formula X, Formula XI, and Formula XII above are depicted as block copolymers (for example, blocks of “a” followed by blocks of “b” followed by blocks of “c”), but it is intended that the polymer can be a random or alternating copolymer (for example, “a” “b” and “c” are either randomly distributed or alternate).

In one embodiment, R²⁹ is

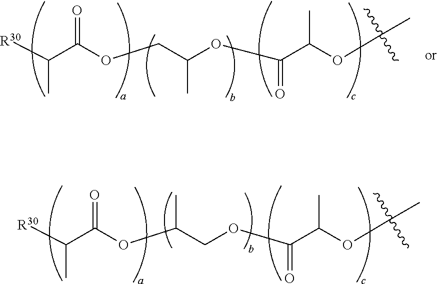

and b is 1.

In one embodiment, R²⁹ is

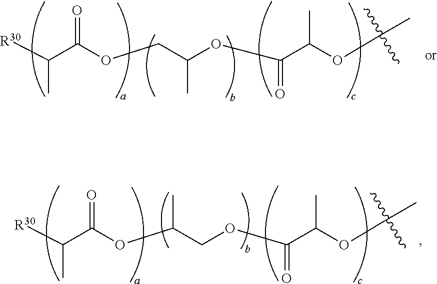

### b is 1, and R³⁰ is

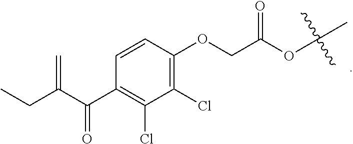 [SMILES: *OC(=O)COc1ccc(C(=O)C(=C)CC)c(Cl)c1Cl]

In certain embodiments, a and c are independently selected from an integer between 1 and 6 (1, 2, 3, 4, 5, or 6) or independently selected from an integer between 1 and 3 (1, 2, or 3).

In one embodiment, a, b, and c are independently selected from an integer between 1 and 12 (1, 2, 3, 4, 5, 6, 7, 8, 9, 10, 11, or 12).

In one embodiment, a, b, and c are independently selected from an integer between 1 and 8 (1, 2, 3, 4, 5, 6, 7, or 8).

In one embodiment, a, b, and c are independently selected from an integer between 1 and 6 (1, 2, 3, 4, 5, or 6).

In one embodiment, a, b, and c are independently selected from an integer between 1 and 3 (1, 2, or 3).

In one e embodiment, a and c are independently selected from an integer between 1 and 6 (1, 2, 3, 4, 5, or 6) and bis 1.

In one embodiment, a and c are independently selected from an integer between 1 and 3 (1, 2, or 3) and b is 1.

In one embodiment, a and c are independently selected from an integer between 1 and 12 (1, 2, 3, 4, 5, 6, 7, 8, 9, 10, 11, or 12) and b is selected from an integer between 1 and 6 (1, 2, 3, 4, 5, or 6).

In one embodiment, a and c are independently selected from an integer between 1 and 6 (1, 2, 3, 4, 5, or 6) and b is selected from an integer between 1 and 3 (1, 2, or 3).

In one embodiment, a and c independently selected from an integer between 1, 2, 3, and 4 and b is 1.

In one embodiment, a and c are 2 and b is 1.

In one embodiment, a and c are 3 and b is 1.

In one embodiment, a and c are 4 and b is 1.

In an alternative embodiment, R³⁰ is selected from

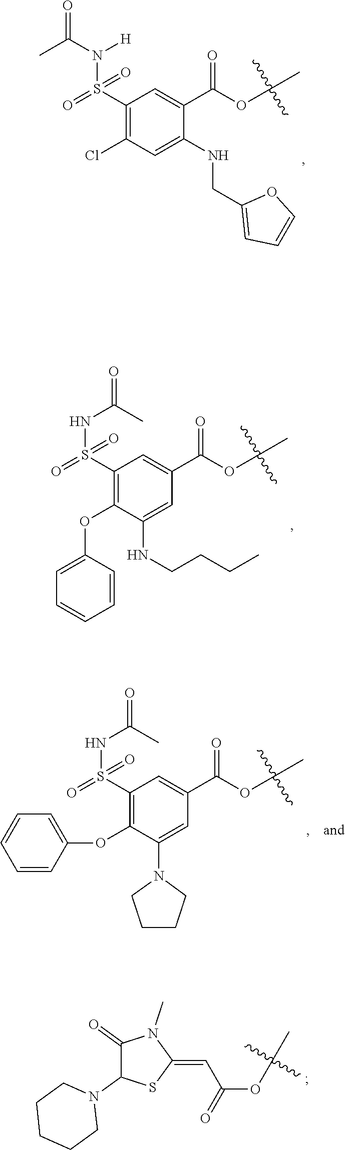 [SMILES: *OC(=O)c1cc(S(=O)(=O)NC(C)=O)c(Cl)cc1NCc1ccco1 ; *OC(=O)c1cc(NCCCC)c(Oc2ccccc2)c(S(=O)(=O)NC(C)=O)c1 ; *OC(=O)c1cc(N2CCCC2)c(Oc2ccccc2)c(S(=O)(=O)NC(C)=O)c1 ; *OC(=O)/C=C1\SC(N2CCCCC2)C(=O)N1C]

The disclosure also provides a prodrug of Formula XIII, Formula XIV, Formula XV, Formula XVI, and Formula XVI′:

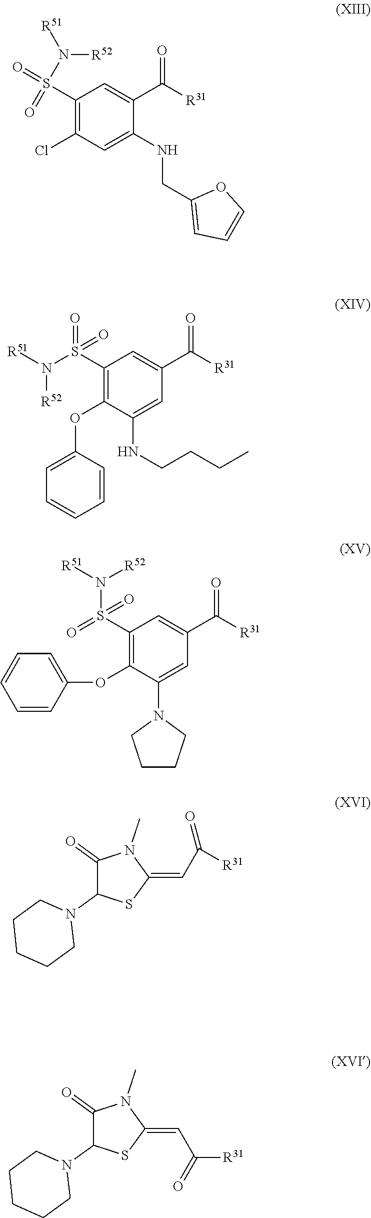

or a pharmaceutically acceptable composition, salt, or isotopic derivative thereof wherein:

R³¹ is selected from

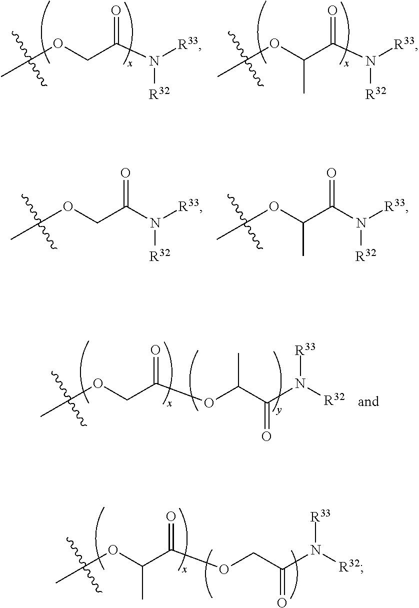

R³² is H, C₁-C₆alkyl, cycloalkyl, cycloalkylalkyl, heterocycle, heterocycloalkyl, aryl, arylalkyl, heteroaryl, or heteroarylalkyl, wherein each group can be optionally substituted with another desired substituent group which is pharmaceutically acceptable and sufficiently stable under the conditions of use, for example selected from R³;

R³³ is hydrogen, C₂-C₆alkyl,

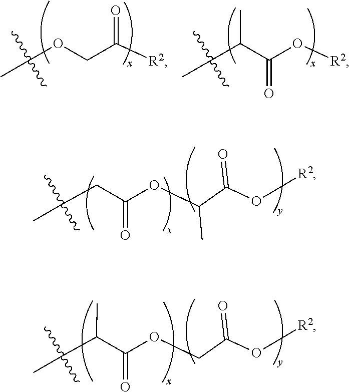

R² is selected at each instance from hydrogen, alkyl, alkenyl, alkynyl cycloalkyl, cycloalkylalkyl, heterocyclyl, heterocycloalkyl, aryl, arylalkyl, heteroaryl, or heteroarylalkyl, each of which except hydrogen may be optionally substituted with R³ if the resulting compound is stable and achieves the desired purpose and wherein the group cannot be substituted with itself, for example alkyl would not be substituted with alkyl;

R³ is selected from halogen, hydroxyl, cyano, mercapto, amino, alkoxy, cycloalkyl, cycloalkylalkyl, heterocyclyl, heterocycloalkyl, aryl, arylalkyl, heteroaryl, heteroarylalkyl, aryloxy, —S(O)₂alkyl, —S(O)alkyl, —P(O)(Oalkyl)₂, B(OH)₂, —Si(CH₃)₃, —COOH, —COOalkyl, and —CONH₂, each of which except halogen, cyano, and —Si(CH₃)₃ may be optionally substituted, for example with halogen, alkyl, aryl, heterocycle or heteroaryl if desired and if the resulting compound is stable and achieves the desired purpose and wherein the group cannot be substituted with itself, for example alkyl would not be substituted with alkyl; and

R⁵¹, R⁵², x, and y are defined herein.

In one embodiment, R³¹ is selected from

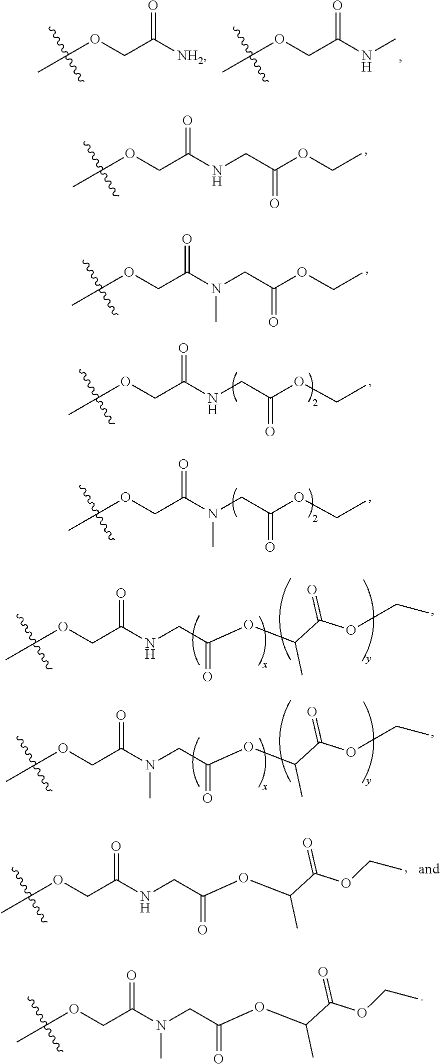

In one embodiment, R³³ is selected from

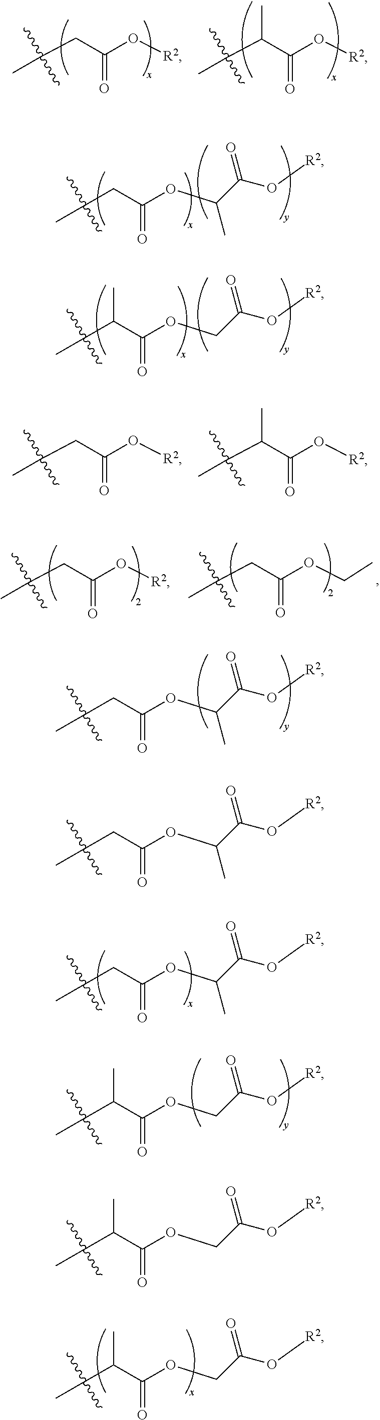

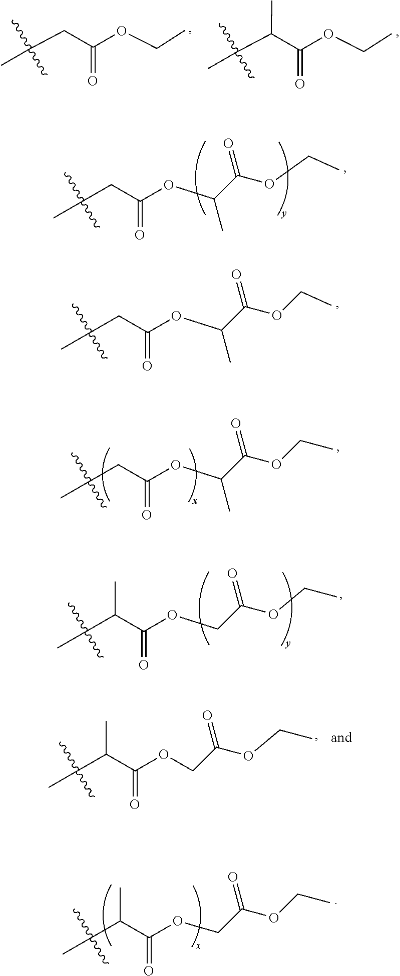

The disclosure also provides a prodrug of Formula XVII, Formula XVII, Formula XIX, Formula XX, and Formula XX′:

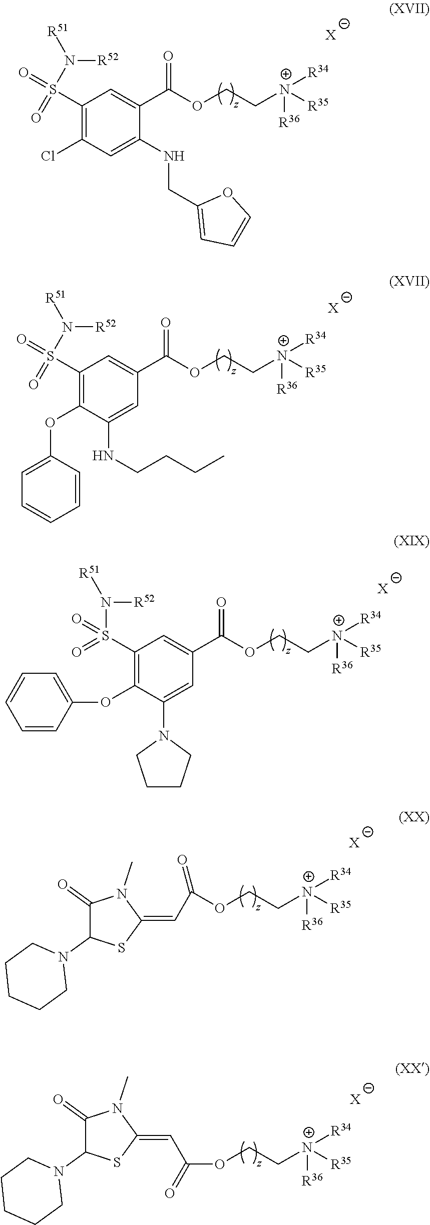

or a pharmaceutically acceptable composition or isotopic derivative thereof
wherein:

R³⁴, R³⁵, and R³⁷ are independently selected from C₁-C₁₂alkyl, aryl, and arylalkyl;

R³⁶ is selected from methyl, C₃-C₁₂alkyl, aryl, and arylalkyl; and

X⁻ is an anion selected from Cl⁻, Br⁻, SO₄²⁻, CH₃CO₂⁻, NO₃⁻;

In one embodiment, R³⁴, R³⁵, and R³⁷ are methyl.
In one embodiment, R³⁶ is methyl.
In one embodiment, the anion is Cl⁻ or Br⁻.

The disclosure also provides a prodrug of Formula XXI, Formula XXII, Formula XXIII, Formula XXIV, or Formula XXIV′:

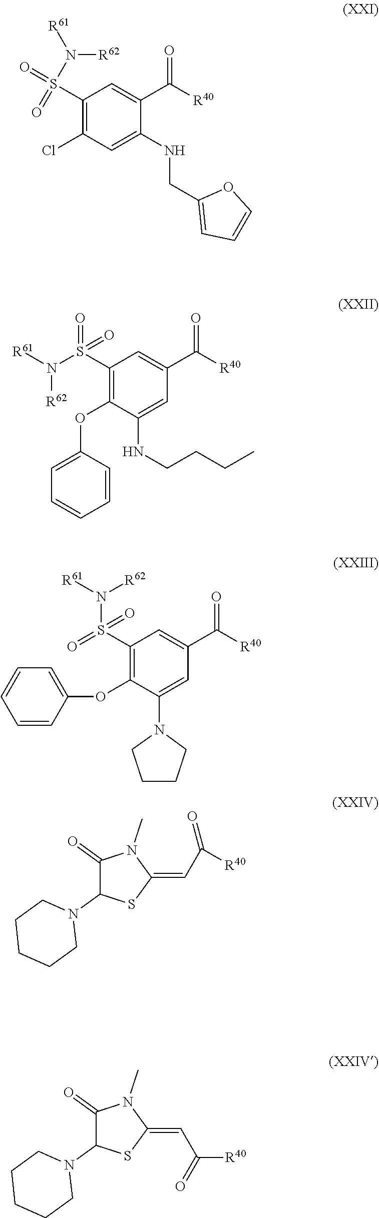

or a pharmaceutically acceptable composition, salt, or isotopic derivative thereof.
wherein:

R⁴⁰ is selected from R⁴¹,

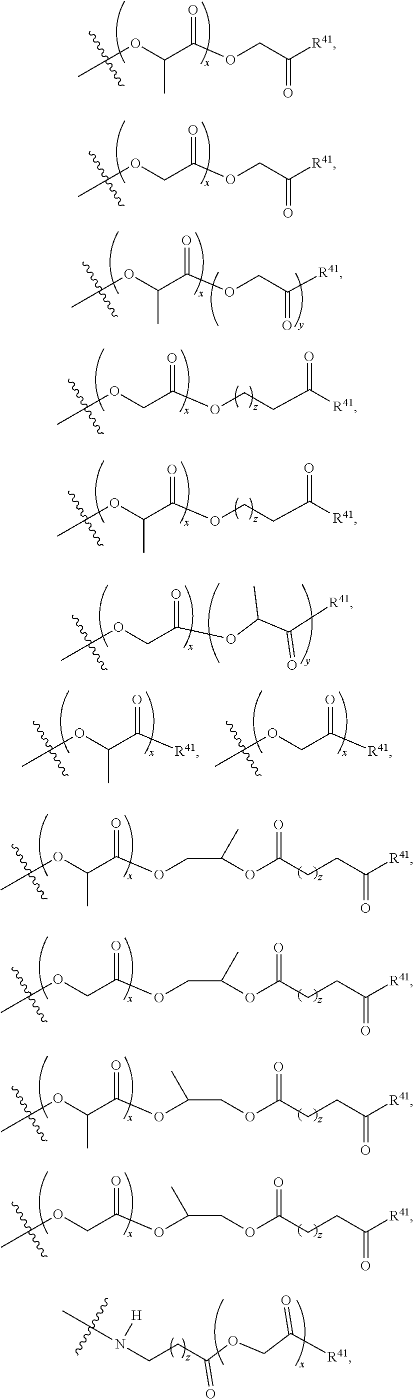

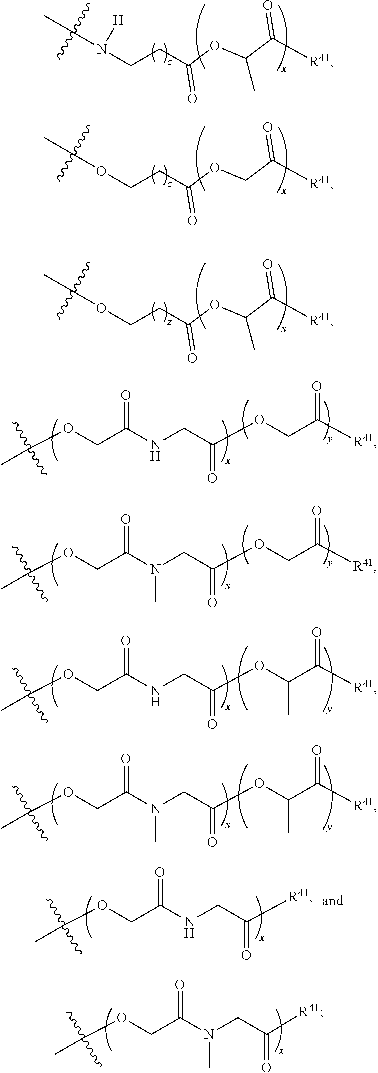

R⁴¹ is independently selected from

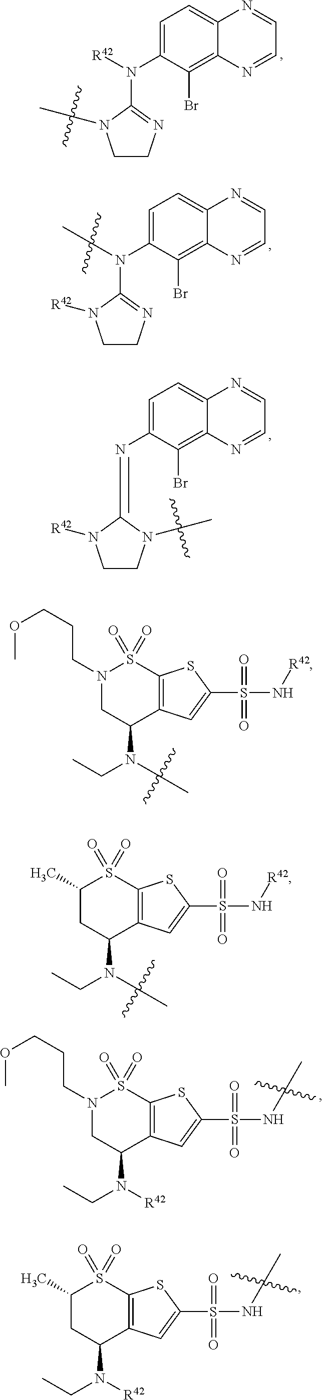

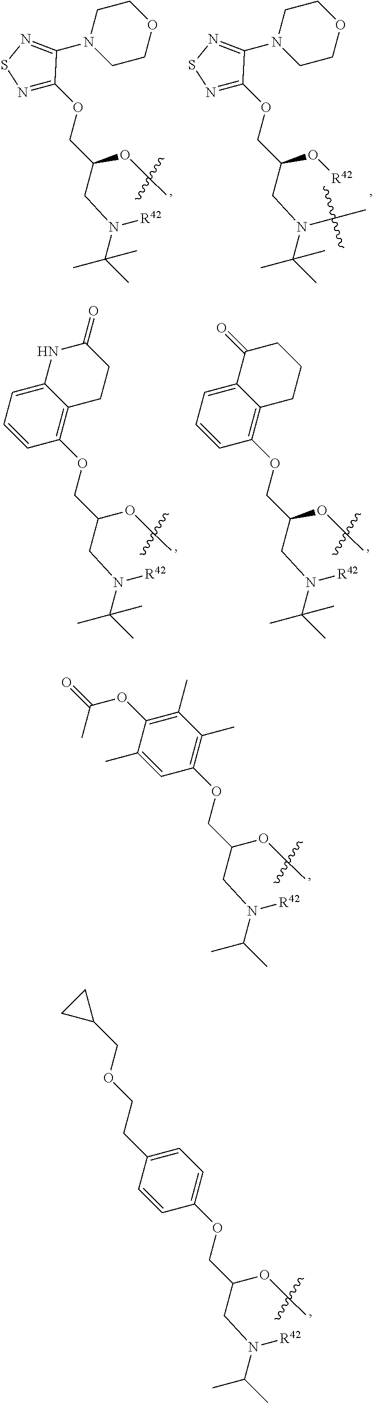

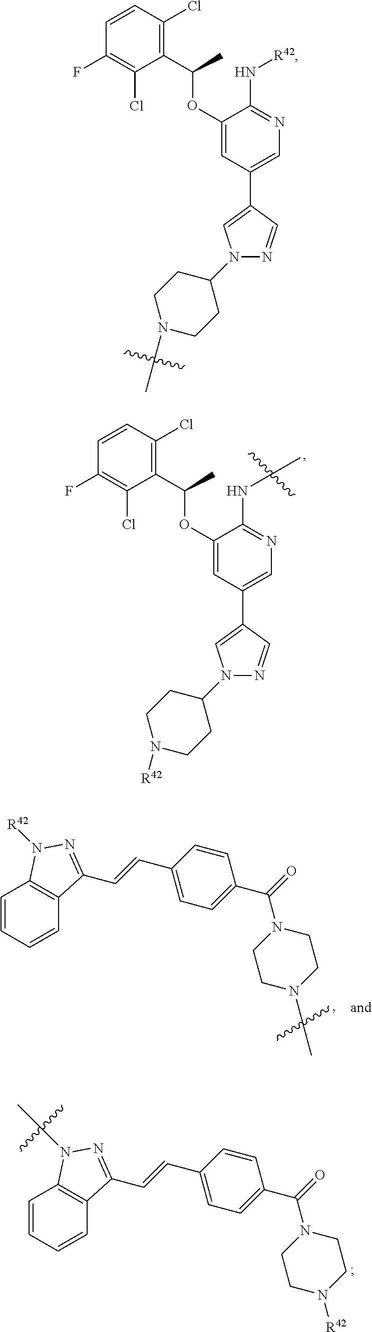

R⁴² is selected from R⁴³,

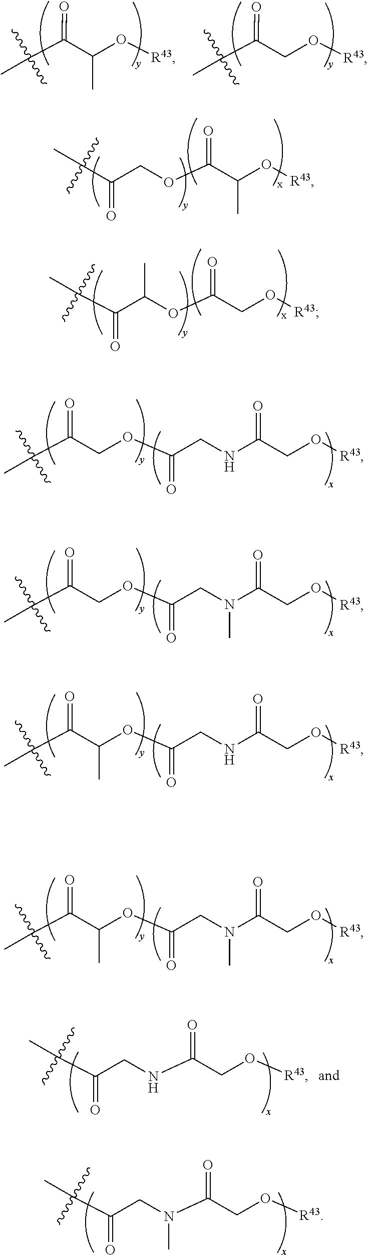

R⁴³ is selected from

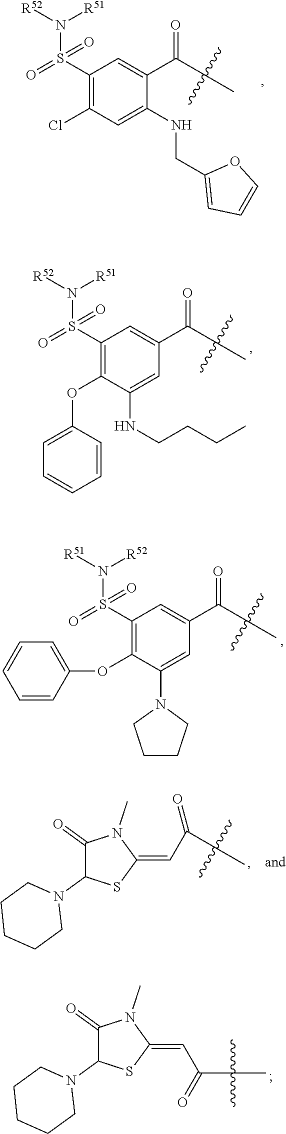

and

R⁵¹, R^{5 2}, R⁶¹, R⁶², x, y, an z are defined herein.

In one embodiment, R⁴⁰ is selected from

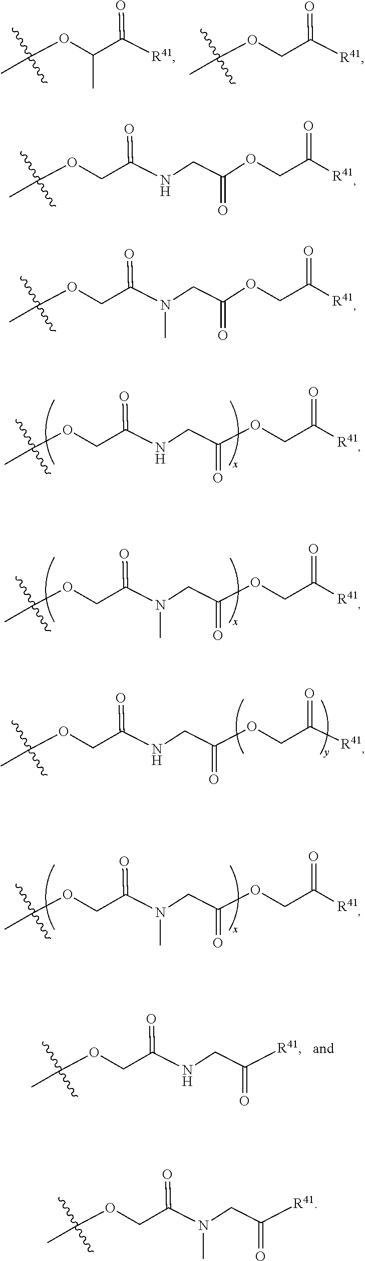

In one embodiment, R⁴² is selected from

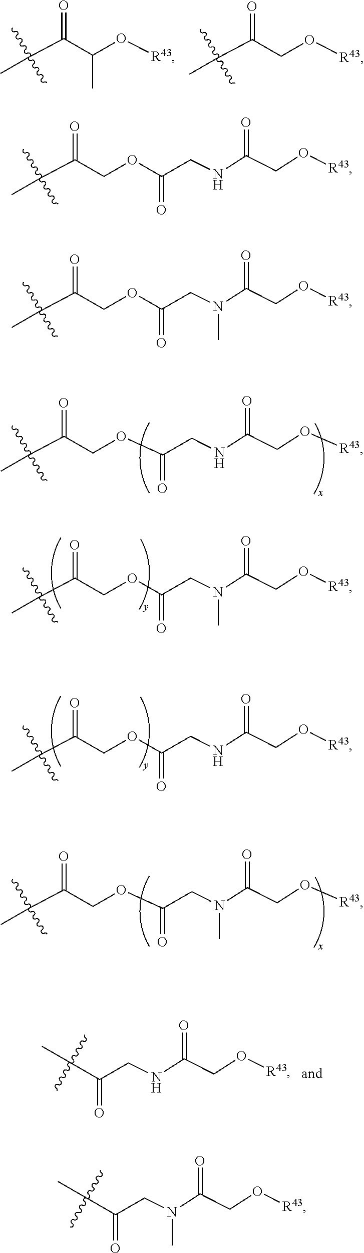

In one embodiment, R⁴⁰ is R⁴¹ and R⁴¹ is

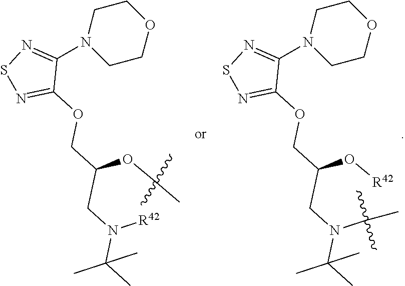

In one embodiment, R⁴⁰ is R⁴¹, R⁴¹ is

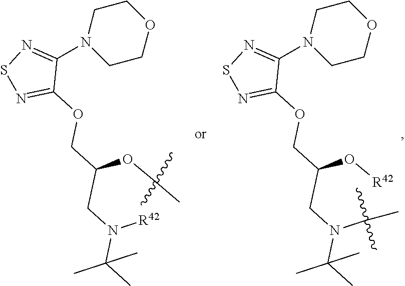

and R⁴² is R⁴³.

In one embodiment the prodrug of Formula I, Formula II, or Formula III is selected from:

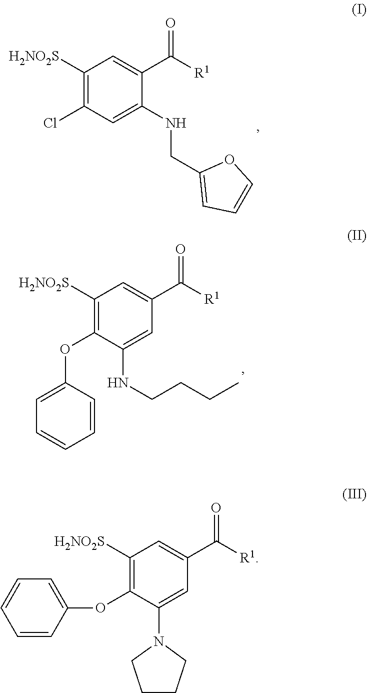

In one embodiment the prodrug of Formula V, Formula VI, or Formula VII is selected from:

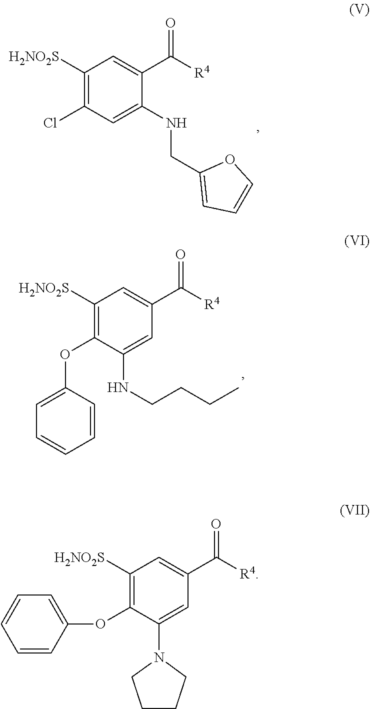

In one alternative embodiment, the prodrug of Formula V, Formula VI, or Formula VII is selected from:

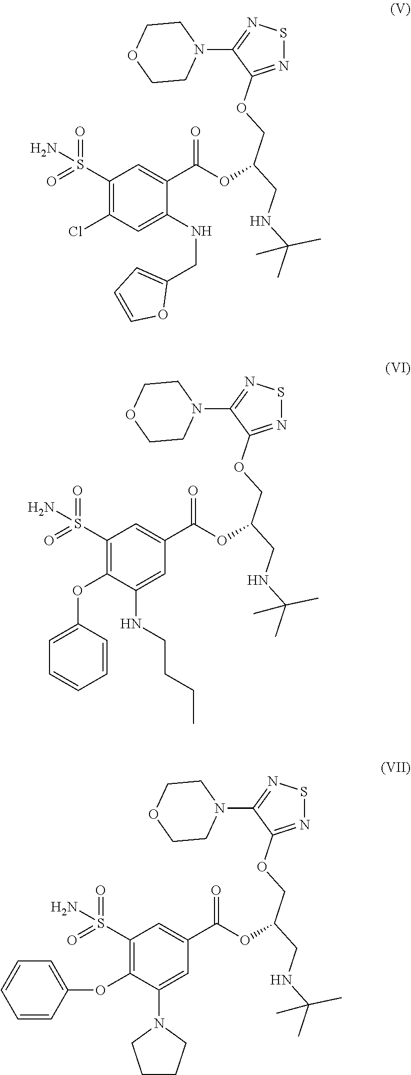 [SMILES: CC(C)(C)NC[C@@H](COc1nsnc1N1CCOCC1)OC(=O)c1cc(S(N)(=O)=O)c(Cl)cc1NCc1ccco1 ; CCCCNc1cc(C(=O)O[C@@H](CNC(C)(C)C)COc2nsnc2N2CCOCC2)cc(S(N)(=O)=O)c1Oc1ccccc1 ; CC(C)(C)NC[C@@H](COc1nsnc1N1CCOCC1)OC(=O)c1cc(N2CCCC2)c(Oc2ccccc2)c(S(N)(=O)=O)c1]

In one embodiment, a compound of Formula V, Formula VI, or Formula VII is the pharmaceutically acceptable succinic acid.

In one embodiment, a compound of Formula V, Formula VI, or Formula VII is the pharmaceutically acceptable tartaric acid.

In one embodiment, a compound of Formula V, Formula VI, or Formula VII is the pharmaceutically acceptable maleic acid.

In one embodiment, a compound of Formula V, Formula VI, or Formula VII is the pharmaceutically acceptable fumaric acid.

In one embodiment the prodrug of Formula IX, Formula X, or Formula XI is selected from:

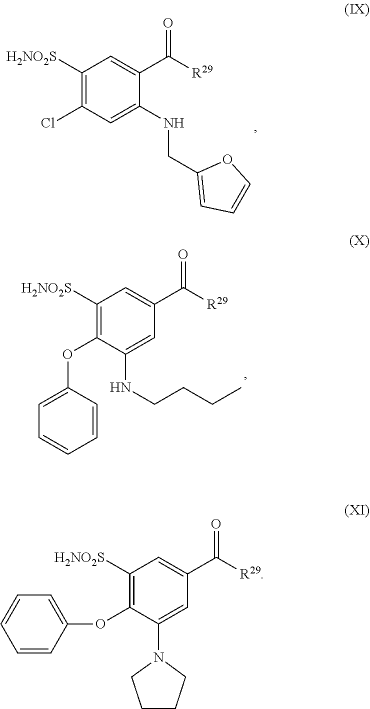

In one embodiment the prodrug of Formula I, Formula II, or Formula III is selected from:

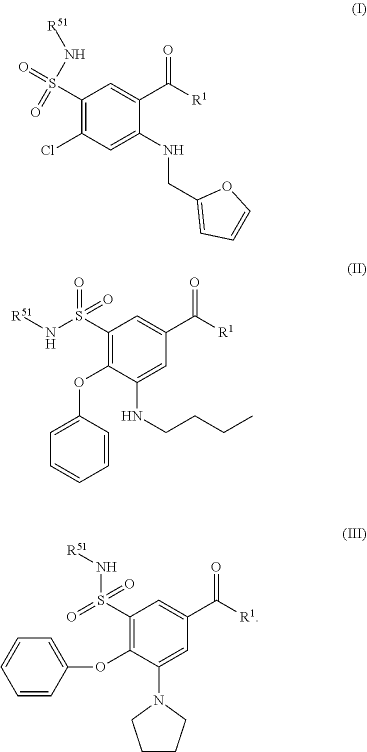

In one embodiment the prodrug of Formula V, Formula VI, or Formula VII is selected from:

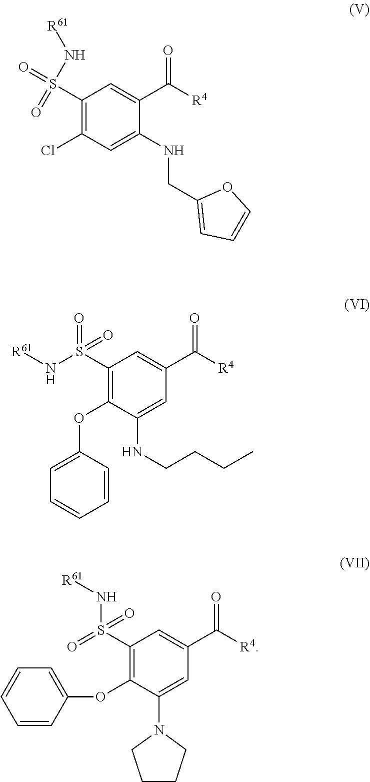

In one embodiment the prodrug of Formula IX, Formula X, or Formula XI is selected from:

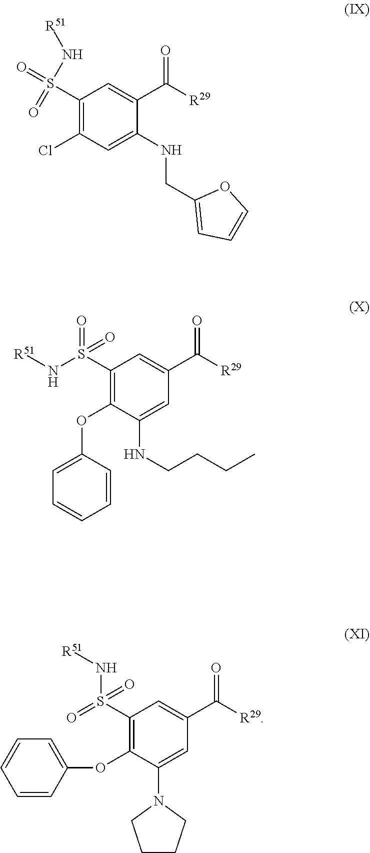

In one embodiment the prodrug of Formula XIII, Formula XIV, or Formula XV is selected

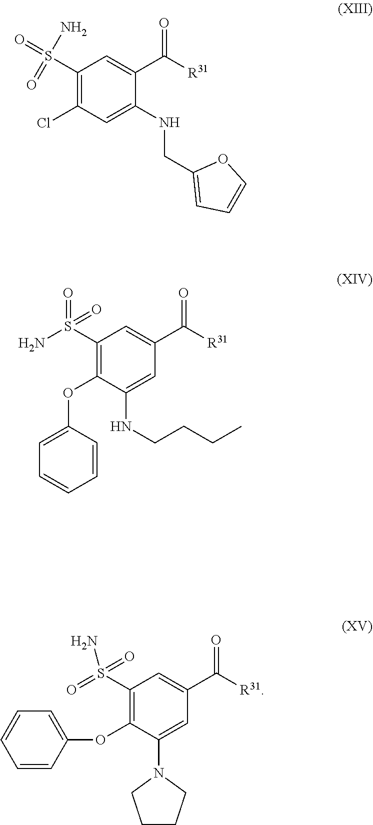

In one embodiment the prodrug of Formula XVII, Formula XVII, or Formula XIX is selected from:

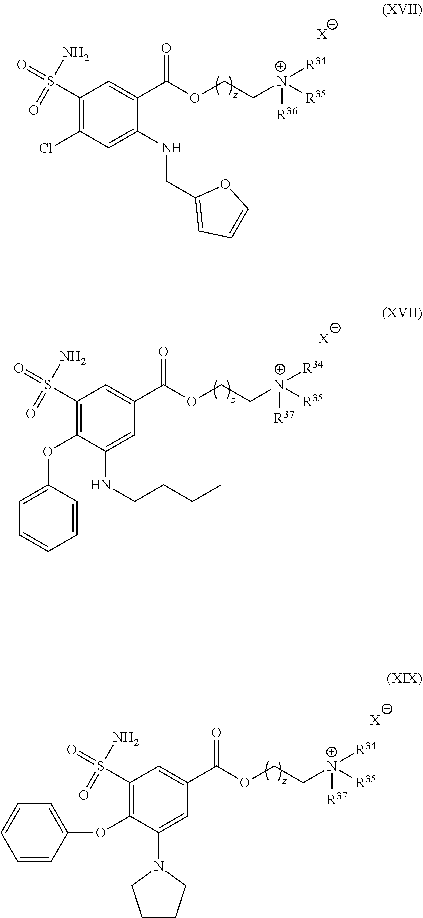

In one embodiment the prodrug of Formula XXI, Formula XXII, or Formula XXIII is selected from:

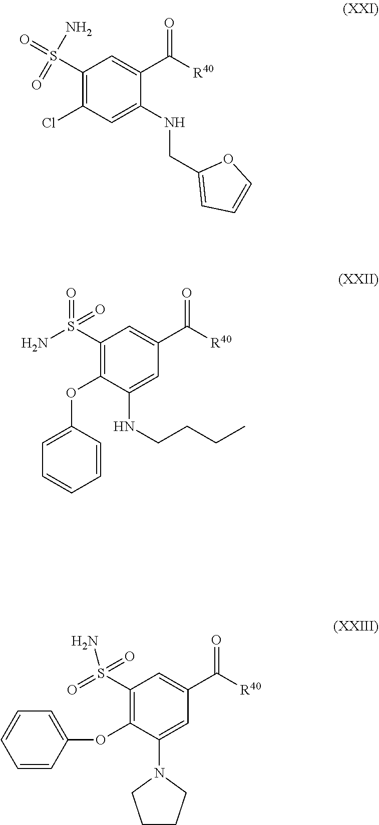

In one embodiment the prodrug of Formula XIII, Formula XIV, or Formula XV is selected from:

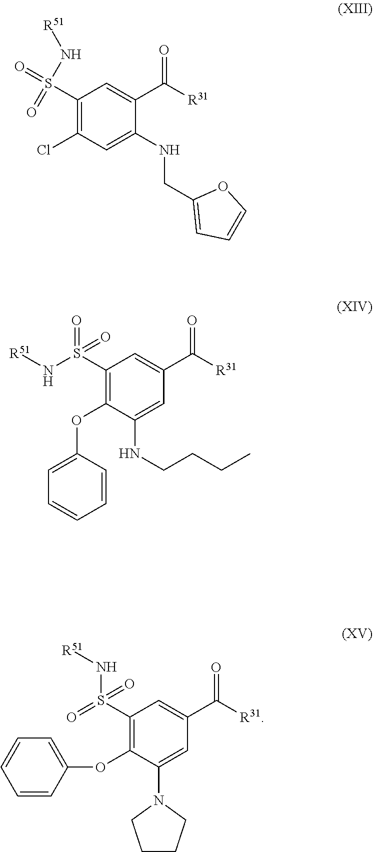

In one embodiment the prodrug of Formula XVII, Formula XVII, or Formula XIX is selected from:

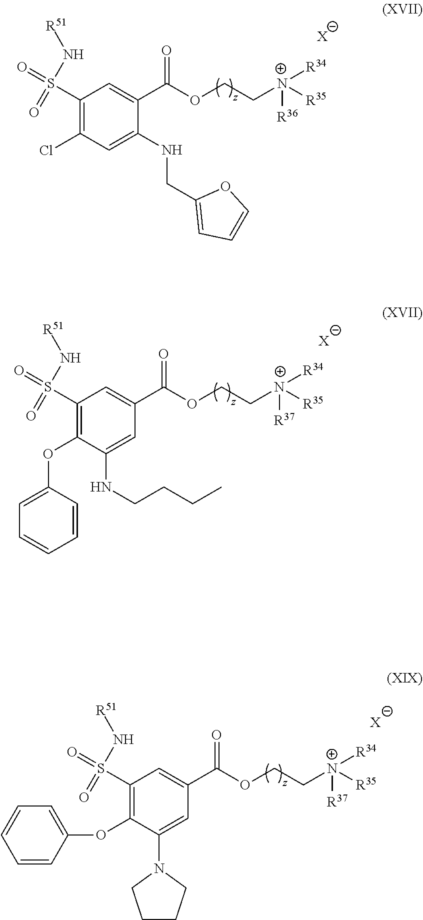

In one embodiment the prodrug of Formula XXI, Formula XXII, or Formula XXIII is selected from:

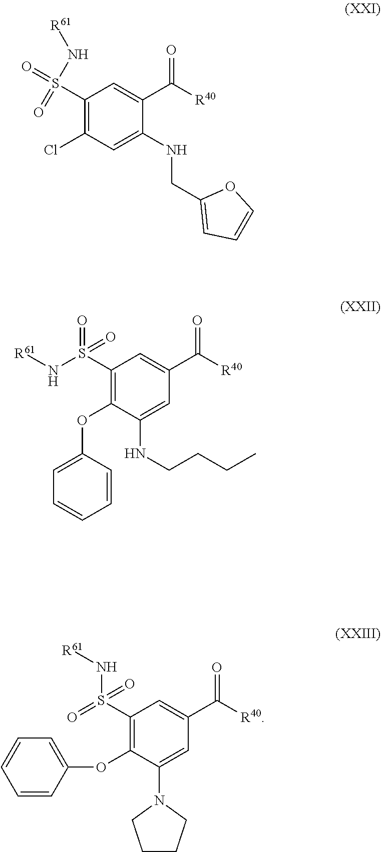

In certain embodiments, R⁵¹ is C(O)A. In one embodiment, R⁵¹ is C(O)CH₃.

In certain embodiments, R⁶¹ is C(O)A. In one embodiment, R⁶¹ is C(O)CH₃.

Pharmaceutical compositions comprising a compound or salt of Formula I, Formula II, Formula III, Formula IV, Formula IV′ Formula V, Formula VI, Formula VII, Formula VIII, Formula VIII′, Formula IX, Formula X, Formula XI, Formula XII, Formula XII′, Formula XIII, Formula XIV, Formula XV, Formula XVI, Formula XVI′, Formula XVII, Formula XVIII, Formula XIX, Formula XX, Formula XX′, Formula XXI, Formula XXII, Formula XXIII, Formula XXIV, or Formula XXIV′ together with a pharmaceutically acceptable carrier are also disclosed.

Methods of treating or preventing ocular disorders, including glaucoma, a disorder mediated by carbonic anhydrase, a disorder mediated by a Rho-associated kinase, a disorder mediated by a dual leucine zipper kinase, a disorder mediated by an α2 adrenergic receptor, a disorder mediated a disorder or abnormality related to an increase in intraocular pressure (IOP), a disorder mediated by nitric oxide synthase (NOS), a disorder requiring neuroprotection such as to regenerate/repair optic nerves, allergic conjunctivitis, anterior uveitis, cataracts, dry or wet age-related macular degeneration (AMD), geographic atrophy, or diabetic retinopathy are disclosed comprising administering a therapeutically effective amount of a compound or salt or Formula I, Formula II, Formula III, Formula IV, Formula IV′ Formula V, Formula VI, Formula VII, Formula VIII, Formula VIII′, Formula IX, Formula X, Formula XI, Formula XII, Formula XII′, Formula XIII, Formula XIV, Formula XV, Formula XVI, Formula XVI′, Formula XVII, Formula XVIII, Formula XIX, Formula XX, Formula XX′, Formula XXI, Formula XXII, Formula XXIII, Formula XXIV, or Formula XXIV′ to a host, including a human, in need of such treatment.

In another embodiment, an effective amount of a compound of Formula I, Formula II, Formula III, Formula IV, Formula IV′ Formula V, Formula VI, Formula VII, Formula VIII, Formula VIII′, Formula IX, Formula X, Formula XI, Formula XII, Formula XII′, Formula XIII, Formula XIV, Formula XV, Formula XVI, Formula XVI′, Formula XVII, Formula XVIII, Formula XIX, Formula XX, Formula XX′, Formula XXI, Formula XXII, Formula XXIII, Formula XXIV, or Formula XXIV′ is provided to decrease intraocular pressure (IOP) caused by glaucoma. In an alternative embodiment, the compound of Formula I, Formula II, Formula III, Formula IV, Formula IV′ Formula V, Formula VI, Formula VII, Formula VIII, Formula VIII′, Formula IX, Formula X, Formula XI, Formula XII, Formula XII′, Formula XIII, Formula XIV, Formula XV, Formula XVI, Formula XVI′, Formula XVII, Formula XVIII, Formula XIX, Formula XX, Formula XX′, Formula XXI, Formula XXII, Formula XXIII, Formula XXIV, or Formula XXIV′ can be used to decrease intraocular pressure (IOP), regardless of whether it is associated with glaucoma.

In one embodiment, the disorder is associated with an increase in intraocular pressure (IOP) caused by potential or previously poor patient compliance to glaucoma treatment. In yet another embodiment, the disorder is associated with potential or poor neuroprotection through neuronal nitric oxide synthase (NOS). The active compound or its salt or prodrug provided herein may thus dampen or inhibit glaucoma in a host, by administration of an effective amount in a suitable manner to a host, typically a human, in need thereof.

Methods for the treatment of a disorder associated with glaucoma, increased intraocular pressure (IOP), and optic nerve damage caused by either high intraocular pressure (IOP) or neuronal nitric oxide synthase (NOS) are provided that includes the administration of an effective amount of a compound Formula I, Formula II, Formula III, Formula IV, Formula IV′ Formula V, Formula VI, Formula VII, Formula VIII, Formula VIII′, Formula IX, Formula X, Formula XI, Formula XII, Formula XII′, Formula XIII, Formula XIV, Formula XV, Formula XVI, Formula XVI′, Formula XVII, Formula XVIII, Formula XIX, Formula XX, Formula XX′, Formula XXI, Formula XXII, Formula XXIII, Formula XXIV, or Formula XXIV′ or a pharmaceutically acceptable salt thereof, optionally in a pharmaceutically acceptable carrier are also disclosed.

Methods for the treatment of a disorder associated with age-related macular degeneration (AMD) and geographic atrophy are provided that includes the administration of an effective amount of a compound Formula I, Formula II, Formula III, Formula IV, Formula IV′ Formula V, Formula VI, Formula VII, Formula VIII, Formula VIII′, Formula IX, Formula X, Formula XI, Formula XII, Formula XII′, Formula XIII, Formula XIV, Formula XV, Formula XVI, Formula XVI′, Formula XVII, Formula XVIII, Formula XIX, Formula XX, Formula XX′, Formula XXI, Formula XXII, Formula XXIII, Formula XXIV, or Formula XXIV′ or a pharmaceutically acceptable salt thereof, optionally in a pharmaceutically acceptable carrier are also disclosed.

Methods for treatment of a disorder mediated by a carbonic anhydrase are provided to treat a patient in need thereof wherein a prodrug of a carbonic anhydrase inhibitor as described herein is provided.

Methods for treatment of a disorder mediated by a Rho-associated kinase are provided to treat a patient in need thereof wherein a prodrug of a Rho-associated kinase inhibitor as described herein is provided.

Methods for treatment of a disorder mediated by a beta-blocker are provided to treat a patient in need thereof wherein a prodrug of a beta blocker as described herein is provided.

Methods for treatment of a disorder mediated by a dual leucine zipper kinase are provided to treat a patient in need thereof wherein a prodrug of a dual leucine zipper kinase inhibitor as described herein is provided.

Methods for treatment of a disorder mediated by a α₂ adrenergic are provided to treat a patient in need thereof also disclosed wherein a prodrug of a α₂ adrenergic agonist as described herein is provided.

The present invention includes at least the following features:
(a) a compound of Formula I, Formula II, Formula III, Formula IV, Formula IV′ Formula V, Formula VI, Formula VII, Formula VIII, Formula VIII′, Formula IX, Formula X, Formula XI, Formula XII, Formula XII′, Formula XIII, Formula XIV, Formula XV, Formula XVI, Formula XVI′, Formula XVII, Formula XVIII, Formula XIX, Formula XX, Formula XX′, Formula XXI, Formula XXII, Formula XXIII, Formula XXIV, or Formula XXIV′ as described herein, or a pharmaceutically acceptable salt or prodrug thereof (each of which and all subgenuses and species thereof are considered individually and specifically described);
(b) a compound of Formula I, Formula II, Formula III, Formula IV, Formula IV′ Formula V, Formula VI, Formula VII, Formula VIII, Formula VIII′, Formula IX, Formula X, Formula XI, Formula XII, Formula XII′, Formula XIII, Formula XIV, Formula XV, Formula XVI, Formula XVI′, Formula XVII, Formula XVIII, Formula XIX, Formula XX, Formula XX′, Formula XXI, Formula XXII, Formula XXIII, Formula XXIV, or Formula XXIV′ as described herein, or a pharmaceutically acceptable salt or prodrug thereof, for use in treating or preventing an ocular disorder as further described herein;
(c) a compound of Formula I, Formula II, Formula III, Formula IV, Formula IV′ Formula V, Formula VI, Formula VII, Formula VIII, Formula VIII′, Formula IX, Formula X, Formula XI, Formula XII, Formula XII′, Formula XIII, Formula XIV, Formula XV, Formula XVI, Formula XVI′, Formula XVII, Formula XVIII, Formula XIX, Formula XX, Formula XX′, Formula XXI, Formula XXII, Formula XXIII, Formula XXIV, or Formula XXIV′ as described herein, or a pharmaceutically acceptable salt or prodrug thereof for use in treating or preventing disorders related to an ocular disorder such as glaucoma, a disorder mediated by carbonic anhydrase, a disorder or abnormality related to an increase in intraocular pressure (IOP), a disorder mediated by nitric oxide synthase (NOS), a disorder requiring neuroprotection such as to regenerate/repair optic nerves, allergic conjunctivitis, anterior uveitis, cataracts, dry or wet age-related macular degeneration (AMD), geographic atrophy or diabetic retinopathy;
(d) use of a compound of Formula I, Formula II, Formula III, Formula IV, Formula IV′ Formula V, Formula VI, Formula VII, Formula VIII, Formula VIII′, Formula IX, Formula X, Formula XI, Formula XII, Formula XII′, Formula XIII, Formula XIV, Formula XV, Formula XVI, Formula XVI′, Formula XVII, Formula XVIII, Formula XIX, Formula XX, Formula XX′, Formula XXI, Formula XXII, Formula XXIII, Formula XXIV, or Formula XXIV′ or a pharmaceutically acceptable salt or prodrug thereof in the manufacture of a medicament for use in treating or preventing glaucoma, wet age-related macular degeneration, dry age-rerated macular degeneration, and disorders involving increased intraocular pressure (IOP) or nerve damage related to either IOP or nitric oxide synthase (NOS) and other disorders described further herein;
(e) use of a compound of Formula I, Formula II, Formula III, Formula IV, Formula IV′ Formula V, Formula VI, Formula VII, Formula VIII, Formula VIII′, Formula IX, Formula X, Formula XI, Formula XII, Formula XII′, Formula XIII, Formula XIV, Formula XV, Formula XVI, Formula XVI′, Formula XVII, Formula XVIII, Formula XIX, Formula XX, Formula XX′, Formula XXI, Formula XXII, Formula XXIII, Formula XXIV, or Formula XXIV′ or a pharmaceutically acceptable salt or prodrug thereof in the manufacture of a medicament for use in treating or preventing age-related macular degeneration (AMD) and other disorders described further herein;
(f) a process for manufacturing a medicament intended for the therapeutic use for treating or preventing glaucoma and disorders involving nerve damage related to both (IOP) and nitric oxide synthase (NOS) and other disorders described further herein characterized in that a compound of Formula I, Formula II, Formula III, Formula IV, Formula IV′ Formula V, Formula VI, Formula VII, Formula VIII, Formula VIII′, Formula IX, Formula X, Formula XI, Formula XII, Formula XII′, Formula XIII, Formula XIV, Formula XV, Formula XVI, Formula XVI′, Formula XVII, Formula XVIII, Formula XIX, Formula XX, Formula XX′, Formula XXI, Formula XXII, Formula XXIII, Formula XXIV, or Formula XXIV′ as described herein is used in the manufacture;
(g) a pharmaceutical formulation comprising an effective host-treating amount of the a compound of Formula I, Formula II, Formula III, Formula IV, Formula IV′ Formula V, Formula VI, Formula VII, Formula VIII, Formula VIII′, Formula IX, Formula X, Formula XI, Formula XII, Formula XII′, Formula XIII, Formula XIV, Formula XV, Formula XVI, Formula XVI′, Formula XVII, Formula XVIII, Formula XIX, Formula XX, Formula XX′, Formula XXI, Formula XXII, Formula XXIII, Formula XXIV, or Formula XXIV′ or a pharmaceutically acceptable salt or prodrug thereof together with a pharmaceutically acceptable carrier or diluent;
(h) a compound of Formula I, Formula II, Formula III, Formula IV, Formula IV′ Formula V, Formula VI, Formula VII, Formula VIII, Formula VIII′, Formula IX, Formula X, Formula XI, Formula XII, Formula XII′, Formula XIII, Formula XIV, Formula XV, Formula XVI, Formula XVI′, Formula XVII, Formula XVIII, Formula XIX, Formula XX, Formula XX′, Formula XXI, Formula XXII, Formula XXIII, Formula XXIV, or Formula XXIV′ as described herein in substantially pure form, (e.g., at least 90 or 95%);
(i) processes for the manufacture of a compound of Formula I, Formula II, Formula III, Formula IV, Formula IV′ Formula V, Formula VI, Formula VII, Formula VIII, Formula VIII′, Formula IX, Formula X, Formula XI, Formula XII, Formula XII′, Formula XIII, Formula XIV, Formula XV, Formula XVI, Formula XVI′, Formula XVII, Formula XVIII, Formula XIX, Formula XX, Formula XX′, Formula XXI, Formula XXII, Formula XXIII, Formula XXIV, or Formula XXIV′ or a pharmaceutically acceptable salt or prodrug thereof; and
(j) processes for the preparation of therapeutic products including drug delivery agents that contain an effective amount a compound of Formula I, Formula II, Formula III, Formula IV, Formula IV′ Formula V, Formula VI, Formula VII, Formula VIII, Formula VIII′, Formula IX, Formula X, Formula XI, Formula XII, Formula XII′, Formula XIII, Formula XIV, Formula XV, Formula XVI, Formula XVI′, Formula XVII, Formula XVIII, Formula XIX, Formula XX, Formula XX′, Formula XXI, Formula XXII, Formula XXIII, Formula XXIV, or Formula XXIV′ as described herein.
(k) A polymeric microparticle comprising a loop diuretic selected from furosemide, bumetanide, piretanide, and etozolin or a pharmaceutically acceptable salt thereof encapsulated in a blend of one or more hydrophobic polymer and an amphiphilic polymer wherein the loop diuretic is released for at least 1 month;
(l) A loop diuretic selected from furosemide, bumetanide, piretanide, and etozolin for use in treating a ocular disorder as further described herein wherein the loop diuretic is administered via intravitreal, intrastromal, intracameral, sub-tenon, sub-retinal, retro-bulbar, peribulbar, suprachoroidal, choroidal, subchoroidal, conjunctival, subconjunctival, episcleral, posterior juxtascleral, circumcorneal, or tear duct injection; and
(m)A polymeric microparticle comprising a compound selected from Compound 26 or Compound 78 or a pharmaceutically acceptable salt thereof encapsulated in a blend of one or more hydrophobic polymer and an amphiphilic polymer wherein the loop diuretic is released for at least 1 month

### BRIEF DESCRIPTION OF FIGURES

FIG. 1 is a graph depicting the stability of bumetanide-ethyl-PLA (n=4) (Compound 2) at 37° C. where 0′ denotes bumetanide (parent), 1′ denotes bumetanide-PLA (n=1), 2′ denotes bumetanide-PLA (n=2), 3′ denotes bumetanide-PLA (n=3) and 4′ denotes bumetanide-ethyl-PLA (n=4) as described in Example 8. The x-axis is time measured in days and the y-axis is the area measured in intensity.

FIG. 2 is a graph depicting the stability of furosemide-ethyl-PLA (n=4) (Compound 1) at 37° C. where 0′ denotes furosemide (parent), 1′ denotes furosemide-PLA (n=1), 2′ denotes furosemide-PLA (n=2), 3′ denotes furosemide-PLA (n=3), and 4′ denotes furosemide-ethyl PLA (n=4) as described in Example 8. The x-axis is time measured in days and the y-axis is the area measured in intensity.

FIG. 3 is a graph depicting the stability of furosemide-ethyl-PLA (n=6) (Compound 5) at 37° C. where 0′ denotes furosemide (parent), 1′ denotes furosemide-PLA (n=1), 2′ denotes furosemide-PLA (n=2), 3′ denotes furosemide-PLA (n=3), and 4′ denotes furosemide-ethyl PLA (n=4) as described in Example 8. The x-axis is time measured in days and the y-axis is the area measured in intensity.

FIG. 4 is a light microscopy image at 40× magnification of microparticles encapsulating furosemide-ethyl-PLA(n=6) (Compound 5) as described in Example 9. Scale bar=50 μm.

FIG. 5 is a graph depicting the drug release kinetics of furosemide-ethyl-PLA (n=6) (Compound 5) and bumetanide-ethyl-PLA(n=4) (Compound 2) from microparticles as described in Example 8.

FIG. 6 is a graph of the IOP reduction following the administration of furosemide or bumetanide via intracameral injection as described in Example 10. Furosemide and bumetanide (5 μg) were dosed intracamerally (IC at 10 μL) on Day 0, and IOP was measured on Day 1 and Day 2 using a TonoVet (iCare, Finland) tonometer. Data are expressed as percentage of IOP reduction from baseline. The x-axis the time measured in days and the y-axis is IOP reduction measured in percent.

FIG. 7 is a graph of the IOP reduction following the administration of furosemide or bumetanide via subconjunctival injection as described in Example 10. Furosemide and bumetanide (5 μg) were dosed subconjunctivally (SC at 20 μL) on Day 0, and IOP was measured on Day 1 and Day 2 using a TonoVet (iCare, Finland) tonometer. Data are expressed as percentage of IOP reduction from baseline. The x-axis the time measured in days and the y-axis is IOP reduction measured in percent.

FIG. 8 are loop diuretic prodrugs of Formula I, Formula II, Formula III, Formula IV, and Formula IV′.

### DETAILED DESCRIPTION

### I. Terminology

The presently disclosed subject matter may be embodied in many different forms and should not be construed as limited to the embodiments set forth herein. Indeed, many modifications and other embodiments of the presently disclosed subject matter will come to mind for one skilled in the art to which the presently disclosed subject matter pertains having the benefit of the teachings presented in the descriptions included herein. Therefore, it is to be understood that the presently disclosed subject matter is not to be limited to the specific embodiments disclosed and that modifications and other embodiments are intended to be included within the scope of the disclosed subject matter.

Although specific terms are employed herein, they are used in a generic and descriptive sense only and not for purposes of limitation. Unless otherwise defined, all technical and scientific terms used herein have the same meaning as commonly understood by one of ordinary skill in the art to which this presently described subject matter belongs.

Compounds are described using standard nomenclature. Unless defined otherwise, all technical and scientific terms used herein have the same meaning as is commonly understood by one of skill in the art to which this invention belongs.

The compounds in any of the Formulas described herein include enantiomers, mixtures of enantiomers, diastereomers, cis/trans isomers, tautomers, racemates and other isomers, such as rotamers, as if each is specifically described.

The compounds in any of the Formulas may be prepared by chiral or asymmetric synthesis from a suitable optically pure precursor or obtained from a racemate or mixture of enantiomers or diastereomers by any conventional technique, for example, by chromatographic resolution using a chiral column, TLC or by the preparation of diastereoisomers, separation thereof and regeneration of the desired enantiomer or diastereomer. See, e.g., “Enantiomers, Racemates and Resolutions,” by J. Jacques, A. Collet, and S. H. Wilen, (Wiley-Interscience, New York, 1981); S. H. Wilen, A. Collet, and J. Jacques, Tetrahedron, 2725 (1977); E. L. Eliel Stereochemistry of Carbon Compounds (McGraw-Hill, N Y, 1962); and S. H. Wilen Tables of Resolving Agents and Optical Resolutions 268 (E. L. Eliel ed., Univ. of Notre Dame Press, Notre Dame, Ind., 1972, Stereochemistry of Organic Compounds, Ernest L. Eliel, Samuel H. Wilen and Lewis N. Manda (1994 John Wiley & Sons, Inc.), and Stereoselective Synthesis A Practical Approach, Mihily Nógrádi (1995 VCH Publishers, Inc., NY, NY).

The terms “a” and “an” do not denote a limitation of quantity, but rather denote the presence of at least one of the referenced item. Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and are independently combinable. All methods described herein can be performed in a suitable order unless otherwise indicated herein or otherwise clearly contradicted by context. The use of examples, or exemplary language (e.g., “such as”), is intended merely to better illustrate the invention and does not pose a limitation on the scope of the invention unless otherwise claimed.

The present invention includes compounds of Formula I, Formula II, Formula III, Formula IV, Formula IV′ Formula V, Formula VI, Formula VII, Formula VIII, Formula VIII′, Formula IX, Formula X, Formula XI, Formula XII, Formula XII′, Formula XIII, Formula XIV, Formula XV, Formula XVI, Formula XVI′, Formula XVII, Formula XVIII, Formula XIX, Formula XX, Formula XX′, Formula XXI, Formula XXII, Formula XXIII, Formula XXIV, or Formula XXIV′ and the use of compounds with at least one desired isotopic substitution of an atom, at an amount above the natural abundance of the isotope, i.e., enriched. Isotopes are atoms having the same atomic number but different mass numbers, i.e., the same number of protons but a different number of neutrons.

Examples of isotopes that can be incorporated into compounds of the invention include isotopes of hydrogen, carbon, nitrogen, oxygen, phosphorus, fluorine, and chlorine, such as 2H, ³H, ¹¹C, ³C, ¹⁴C, ¹⁵N, ¹⁸F ³¹P, ³²P, ³⁵S, ³⁶Cl, ¹²⁵I respectively. The invention includes isotopically modified compounds of Formula I, Formula II, Formula III, Formula IV, Formula IV′ Formula V, Formula VI, Formula VII, Formula VIII, Formula VIII′, Formula IX, Formula X, Formula XI, Formula XII, Formula XII′, Formula XIII, Formula XIV, Formula XV, Formula XVI, Formula XVI′, Formula XVII, Formula XVIII, Formula XIX, Formula XX, Formula XX′, Formula XXI, Formula XXII, Formula XXIII, Formula XXIV, or Formula XXIV′. Isotopically labeled compounds of this invention and prodrugs thereof can generally be prepared by carrying out the procedures disclosed in the schemes or in the examples and preparations described below by substituting an isotopically labeled reagent for a non-isotopically labeled reagent.

By way of general example and without limitation, isotopes of hydrogen, for example, deuterium (²H) and tritium (3H) may be used anywhere in described structures that achieves the desired result. Alternatively or in addition, isotopes of carbon, e.g., ¹³C and ¹⁴C, may be used. In one embodiment, the isotopic substitution is deuterium for hydrogen at one or more locations on the molecule to improve the performance of the drug, for example, the pharmacodynamics, pharmacokinetics, biodistribution, half-life, stability, AUC, Tₘₐₓ, Cₘₐₓ, etc. For example, the deuterium can be bound to carbon in a location of bond breakage during metabolism (an α-deuterium kinetic isotope effect) or next to or near the site of bond breakage (a β-deuterium kinetic isotope effect).

Isotopic substitutions, for example deuterium substitutions, can be partial or complete. Partial deuterium substitution means that at least one hydrogen is substituted with deuterium. In certain embodiments, the isotope is 90, 95 or 99% or more enriched at any location of interest. In one embodiment deuterium is 90, 95 or 99% enriched at a desired location.

In one embodiment, the substitution of a hydrogen atom for a deuterium atom can be provided in any of A, QL¹, or L². In one embodiment, the substitution of a hydrogen atom for a deuterium atom occurs within an R group selected from any of R¹, R², R^{2′}, R³, R⁴, R⁵, R⁶, R⁷, R⁸, R⁹, R¹, R¹¹, R^{11′}, R¹², R¹³, R¹⁴, R¹⁵, R¹⁶, R¹⁷, R¹⁸, R¹⁹, R²⁰, R²¹, R²², R²³, R²⁴, R², R²⁶, R²⁷, R²⁸, R²⁹, R³⁰, R³¹, R³², R³³, R³⁴, R³⁵, R³⁶, R³⁷, R⁴⁰, R⁴¹, R⁴², and R⁴³. For example, when any of R groups are, or contain for example through substitution, methyl, ethyl, or methoxy, the alkyl residue may be deuterated (in non-limiting embodiments, CD₃, CH₂CD₃, CD₂CD₃, CDH₂, CD₂H, CD₃, CHDCH₂D, CH₂CD₃, CHDCHD₂, OCDH₂, OCD₂H, or OCD₃ etc.

The compound of the present invention may form a solvate with a solvent (including water). Therefore, in one embodiment, the invention includes a solvated form of the active compound. The term “solvate” refers to a molecular complex of a compound of the present invention (including salts thereof) with one or more solvent molecules. Examples of solvents are water, ethanol, dimethyl sulfoxide, acetone and other common organic solvents. The term “hydrate” refers to a molecular complex comprising a compound of the invention and water. Pharmaceutically acceptable solvates in accordance with the invention include those wherein the solvent may be isotopically substituted, e.g. D₂O, d₆-acetone, d₆-DMSO. A solvate can be in a liquid or solid form.

A dash (“-”) is defined by context and can in addition to its literary meaning indicate a point of attachment for a substituent. For example, —(C═O)NH₂ is attached through carbon of the keto (C═O) group. A dash (“-”) can also indicate a bond within a chemical structure. For example —C(O)—NH₂ is attached through carbon of the keto group which is bound to an amino group (NH₂).

An equal sign (“=”) is defined by context and can in addition to its literary meaning indicate a point of attachment for a substituent wherein the attachment is through a double bond. For example, =CH₂ represents a fragment that is doubly bonded to the parent structure and consists of one carbon with two hydrogens bonded in a terminal fashion. ═CHCH₃ on the other hand represents a fragment that is doubly bonded to the parent structure and consists of two carbons. In the above example it should be noted that the stereoisomer is not delineated and that both the cis and trans isomer are independently represented by the group.

The term “substituted”, as used herein, means that any one or more hydrogens on the designated atom or group is replaced with a moiety selected from the indicated group, provided that the designated atom's normal valence is not exceeded. For example, when the substituent is oxo (i.e., =O), then in one embodiment, two hydrogens on the atom are replaced. When an oxo group replaces two hydrogens in an aromatic moiety, the corresponding partially unsaturated ring replaces the aromatic ring. For example a pyridyl group substituted by oxo is a 78ydroxyl. Combinations of substituents and/or variables are permissible only if such combinations result in stable compounds or useful synthetic intermediates.

A stable compound or stable structure refers to a compound with a long enough residence time to either be used as a synthetic intermediate or as a therapeutic agent, as relevant in context.

“Alkyl” is a straight chain saturated aliphatic hydrocarbon group. In certain embodiments, the alkyl is C₁-C₂, C₁-C₃, C₁-C₆, or C₁-C₃₀ (i.e., the alkyl chain can be 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 carbons in length). The specified ranges as used herein indicate an alkyl group with length of each member of the range described as an independent species. For example, the term C₁-C₆ alkyl as used herein indicates a straight alkyl group having from 1, 2, 3, 4, 5, or 6 carbon atoms and is intended to mean that each of these is described as an independent species. For example, the term C₁-C₄alkyl as used herein indicates a straight or branched alkyl group having from 1, 2, 3, or 4 carbon atoms and is intended to mean that each of these is described as an independent species. When C₀-Cₙ alkyl is used herein in conjunction with another group, for example, (C₃-C₇cycloalkyl)C₀-C₄ alkyl, or —C₀-C₄alkyl(C₃-C₇cycloalkyl), the indicated group, in this case cycloalkyl, is either directly bound by a single covalent bond (C₀alkyl), or attached by an alkyl chain in this case 1, 2, 3, or 4 carbon atoms. Alkyls can also be attached via other groups such as heteroatoms as in —O—C₀-C₄alkyl(C₃-C₇cycloalkyl). Alkyls can be further substituted with alkyl to make branched alkyls. Examples of alkyl include, but are not limited to, methyl, ethyl, n-propyl, isopropyl, n-butyl, isobutyl, sec-butyl, t-butyl, n-pentyl, isopentyl, tert-pentyl, neopentyl, n-hexyl, 2-methylpentane, 3-methylpentane, 2,2-dimethylbutane and 2,3-dimethylbutane. In one embodiment, the alkyl group is optionally substituted as described above.

“Alkenyl” is a straight chain aliphatic hydrocarbon group having one or more carbon-carbon double bonds each of which is independently either cis or trans that may occur at a stable point along the chain. In one embodiment, the double bond in a long chain similar to a fatty acid has the stereochemistry as commonly found in nature. Non-limiting examples are C₂-C₃₀alkenyl, C₁-C₃₀alkenyl (i.e., having 2, 3, 4, 5, 6, 7, 8, 9, 10, 11, 12, 13, 14, 15, 16, 17, 18, 19, 20, 21, 22, 23, 24, 25, 26, 27, 28, 29, or 30 carbons), and C₂-C₄alkenyl. The specified ranges as used herein indicate an alkenyl group having each member of the range described as an independent species, as described above for the alkyl moiety. Examples of alkenyl include, but are not limited to, ethenyl and propenyl. Alkenyls can be further substituted with alkyl to make branched alkenyls. In one embodiment, the alkenyl group is optionally substituted as described above.

“Alkynyl” is a straight chain aliphatic hydrocarbon group having one or more carbon-carbon triple bonds that may occur at any stable point along the chain, for example, C₂-C₈alkynyl or C₁₀-C₃₀alkynyl (i.e., having 2, 3, 4, 5, 6, 7, 8, 9, 10, 11, 12, 13, 14, 15, 16, 17, 18, 19, 20, 21, 22, 23, 24, 25, 26, 27, 28, 29, or 30 carbons). The specified ranges as used herein indicate an alkynyl group having each member of the range described as an independent species, as described above for the alkyl moiety. Alkynyls can be further substituted with alkyl to make branched alkynyls. Examples of alkynyl include, but are not limited to, ethynyl, propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl and 5-hexynyl. In one embodiment, the alkynyl group is optionally substituted as described above.

“Alkylene” is a bivalent saturated hydrocarbon. Alkylenes, for example, can be a 1 to 8 carbon moiety, 1 to 6 carbon moiety, or an indicated number of carbon atoms, for example C₁-C₄alkylene, C₁-C₃alkylene, or C₁-C₂alkylene.

“Alkenylene” is a bivalent hydrocarbon having at least one carbon-carbon double bond. Alkenylenes, for example, can be a 2 to 8 carbon moiety, 2 to 6 carbon moiety, or an indicated number of carbon atoms, for example C₂-C₄alkenylene.

“Alkynylene” is a bivalent hydrocarbon having at least one carbon-carbon triple bond. Alkynylenes, for example, can be a 2 to 8 carbon moiety, 2 to 6 carbon moiety, or an indicated number of carbon atoms, for example C₂-C₄alkynylene.

“Alkenylalkynyl” in one embodiment is a bivalent hydrocarbon having at least one carbon-carbon double bond and at least one carbon-carbon triple bond. It will be recognized to one skilled in the art that the bivalent hydrocarbon will not result in hypervalency, for example, hydrocarbons that include —C═C≡C—C or —C≡C≡C—C, and must be stable. Alkenylalkynyls, for example, can be a 4 to 8 carbon moiety, 4 to 6 carbon moiety, or an indicated number of carbon atoms, for example C₄-C₆alkenylalkynyls.

“Alkoxy” is an alkyl group as defined above covalently bound through an oxygen bridge (—O—). Examples of alkoxy include, but are not limited to, methoxy, ethoxy, n-propoxy, i-propoxy, n-butoxy, 2-butoxy, t-butoxy, n-pentoxy, 2-pentoxy, 3-pentoxy, isopentoxy, neopentoxy, n-hexoxy, 2-hexoxy, 3-hexoxy, and 3-methylpentoxy. Similarly an “alkylthio” or a “thioalkyl” group is an alkyl group as defined above with the indicated number of carbon atoms covalently bound through a sulfur bridge (—S—). In one embodiment, the alkoxy group is optionally substituted as described above.

“Alkenyloxy” is an alkenyl group as defined covalently bound to the group it substitutes by an oxygen bridge (—O—).

“Amide” or “carboxamide” is —C(O)NR^{a}R^{b} wherein R^{a} and R^{b} are each independently selected from hydrogen, alkyl, for example, C₁-C₆alkyl, alkenyl, for example, C₂-C₆alkenyl, alkynyl, for example, C₂-C₆alkynyl, —C₀-C₄alkyl(C₃-C₇cycloalkyl), —C₀-C₄alkyl(C₃-C₇heterocycloalkyl), —C₀-C₄alkyl(aryl), and —C₀-C₄alkyl(heteroaryl); or together with the nitrogen to which they are bonded, R^{a} and R^{b} can form a C₃-C₇heterocyclic ring. In one embodiment, the R^{a} and R^{b} groups are each independently optionally substituted as described above.

“Carbocyclic group”, “carbocyclic ring”, or “cycloalkyl” is a saturated or partially unsaturated (i.e., not aromatic) group containing all carbon ring atoms. A carbocyclic group typically contains 1 ring of 3 to 7 carbon atoms or 2 fused rings each containing 3 to 7 carbon atoms. Cycloalkyl substituents may be pendant from a substituted nitrogen or carbon atom, or a substituted carbon atom that may have two substituents can have a cycloalkyl group, which is attached as a spiro group. Examples of carbocyclic rings include cyclohexenyl, cyclohexyl, cyclopentenyl, cyclopentyl, cyclobutenyl, cyclobutyl and cyclopropyl rings. In one embodiment, the carbocyclic ring is optionally substituted as described above. In one embodiment, the cycloalkyl is a partially unsaturated (i.e., not aromatic) group containing all carbon ring atoms. In another embodiment, the cycloalkyl is a saturated group containing all carbon ring atoms. In another embodiment, a carbocyclic ring comprises a caged carbocyclic group. In one embodiment, a carbocyclic ring comprises a bridged carbocyclic group. An example of a caged carbocyclic group is 81ydroxy181e. An example of a bridged carbocyclic group includes 81ydroxy[2.2.1]heptane (norbornane). In one embodiment, the caged carbocyclic group is optionally substituted as described above. In one embodiment, the bridged carbocyclic group is optionally substituted as described above.

“Hydroxyalkyl” is an alkyl group as previously described, substituted with at least one hydroxyl substituent.

“Halo” or “halogen” indicates independently any of fluoro, chloro, bromo, and iodo.

“Aryl” indicates aromatic groups containing only carbon in the aromatic ring or rings. In one embodiment, the aryl groups contain 1 to 3 separate or fused rings and is 6 to about 14 or 18 ring atoms, without heteroatoms as ring members. When indicated, such aryl groups may be further substituted with carbon or non-carbon atoms or groups. Such substitution may include fusion to a 4 to 7-membered saturated cyclic group that optionally contains 1 or 2 heteroatoms independently chosen from N, O, B, and S, to form, for example, a 3,4-methylenedioxyphenyl group. Aryl groups include, for example, phenyl and naphthyl, including 1-naphthyl and 2-naphthyl. In one embodiment, aryl groups are pendant. An example of a pendant ring is a phenyl group substituted with a phenyl group. In one embodiment, the aryl group is optionally substituted as described above. In one embodiment, aryl groups include, for example, dihydroindole, dihydrobenzofuran, isoindoline-1-one and indolin-2-one that can be optionally substituted.

The term “heterocycle,” or “heterocyclic ring” as used herein refers to a saturated or a partially unsaturated (i.e., having one or more double and/or triple bonds within the ring without aromaticity) carbocyclic radical of 3 to about 12, and more typically 3, 5, 6, 7 to 10 ring atoms in which at least one ring atom is a heteroatom selected from nitrogen, oxygen, phosphorus, silicon, boron and sulfur, the remaining ring atoms being C, where one or more ring atoms is optionally substituted independently with one or more substituents described above. A heterocycle may be a monocycle having 3 to 7 ring members (2 to 6 carbon atoms and 1 to 4 heteroatoms selected from N, O, P, and S) or a bicycle having 5 to 10 ring members (4 to 9 carbon atoms and 1 to 6 heteroatoms selected from N, O, P, and S), for example: a 82ydroxy [4,5], [5,5], [5,6], or [6,6] system. In one embodiment, the only heteroatom is nitrogen. In one embodiment, the only heteroatom is oxygen. In one embodiment, the only heteroatom is sulfur. Heterocycles are described in Paquette, Leo A.; “Principles of Modern Heterocyclic Chemistry” (W. A. Benjamin, New York, 1968), particularly Chapters 1, 3, 4, 6, 7, and 9; “The Chemistry of Heterocyclic Compounds, A series of Monographs” (John Wiley & Sons, New York, 1950 to present), in particular Volumes 13, 14, 16, 19, and 28; and J. Am. Chem. Soc. (1960) 82:5566. Spiro moieties are also included within the scope of this definition. Examples of a heterocyclic group wherein 1 or 2 ring carbon atoms are substituted with oxo (=O) moieties are pyrimidinonyl and 1,1-dioxo-thiomorpholinyl. The heterocycle groups herein are optionally substituted independently with one or more substituents described herein.

“Heteroaryl” refers to a stable monocyclic, bicyclic, or multicyclic aromatic ring which contains from 1 to 3, or in some embodiments from 1, 2, or 3 heteroatoms selected from N, O, S, B or P with remaining ring atoms being carbon, or a stable bicyclic or tricyclic system containing at least one 5, 6, or 7 membered aromatic ring which contains from 1 to 3, or in some embodiments from 1 to 2, heteroatoms selected from N, O, S, B or P with remaining ring atoms being carbon. In one embodiment, the only heteroatom is nitrogen. In one embodiment, the only heteroatom is oxygen. In one embodiment, the only heteroatom is sulfur. Monocyclic heteroaryl groups typically have from 5, 6, or 7 ring atoms. In some embodiments bicyclic heteroaryl groups are 8- to 10-membered heteroaryl groups, that is, groups containing 8 or 10 ring atoms in which one 5, 6, or 7 member aromatic ring is fused to a second aromatic or non-aromatic ring. When the total number of S and O atoms in the heteroaryl group exceeds 1, these heteroatoms are not adjacent to one another. In one embodiment, the total number of S and O atoms in the heteroaryl group is not more than 2. In another embodiment, the total number of S and O atoms in the aromatic heterocycle is not more than 1. Examples of heteroaryl groups include, but are not limited to, pyridinyl (including, for example, 2-hydroxypyridinyl), imidazolyl, imidazopyridinyl, pyrimidinyl (including, for example, 4-hydroxypyrimidinyl), pyrazolyl, triazolyl, pyrazinyl, tetrazolyl, furyl, thienyl, isoxazolyl, thiazolyl, oxadiazolyl, oxazolyl, isothiazolyl, pyrrolyl, quinolinyl, isoquinolinyl, tetrahydroisoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, triazolyl, thiadiazolyl, thiadiazolyl, furazanyl, benzofurazanyl, benzothiophenyl, benzothiazolyl, benzoxazolyl, quinazolinyl, quinoxalinyl, naphthyridinyl, tetrahydrofuranyl, and furopyridinyl.

“Heterocycloalkyl” is a saturated ring group. It may have, for example, 1, 2, 3, or 4 heteroatoms independently chosen from N, S, and O, with remaining ring atoms being carbon. In a typical embodiment, nitrogen is the heteroatom. Monocyclic heterocycloalkyl groups typically have from 3 to about 8 ring atoms or from 4 to 6 ring atoms. Examples of heterocycloalkyl groups include morpholinyl, piperazinyl, piperidinyl, and pyrrolinyl.

The term “esterase” refers to an enzyme that catalyzes the hydrolysis of an ester. As used herein, the esterase can catalyze the hydrolysis of prostaglandins described herein. In certain instances, the esterase includes an enzyme that can catalyze the hydrolysis of amide bonds of prostaglandins.

A “dosage form” means a unit of administration of an active agent. Examples of dosage forms include tablets, capsules, injections, suspensions, liquids, emulsions, implants, particles, spheres, creams, ointments, suppositories, inhalable forms, transdermal forms, buccal, sublingual, topical, gel, mucosal, and the like. A “dosage form” can also include an implant, for example an optical implant.

A “pharmaceutical composition” is a composition comprising at least one active agent, such as a compound or salt of Formula I, Formula II, Formula III, Formula IV, Formula IV′ Formula V, Formula VI, Formula VII, Formula VIII, Formula VIII′, Formula IX, Formula X, Formula XI, Formula XII, Formula XII′, Formula XIII, Formula XIV, Formula XV, Formula XVI, Formula XVI′, Formula XVII, Formula XVIII, Formula XIX, Formula XX, Formula XX′, Formula XXI, Formula XXII, Formula XXIII, Formula XXIV, or Formula XXIV′, and at least one other substance, such as a pharmaceutically acceptable carrier. “Pharmaceutical combinations” are combinations of at least two active agents which may be combined in a single dosage form or provided together in separate dosage forms with instructions that the active agents are to be used together to treat any disorder described herein. In one embodiment, the pharmaceutical composition is in a dosage form suitable for topical administration to the eye. In one embodiment, the pharmaceutical composition is a suspension, solution, ointment, or emulsion.

A “pharmaceutically acceptable salt” includes a derivative of the disclosed compound in which the parent compound is modified by making inorganic and organic, suitably non-toxic, acid or base addition salts thereof. The salts of the present compounds can be synthesized from a parent compound that contains a basic or acidic moiety by conventional chemical methods. Generally, such salt can be prepared by reacting free acid forms of these compounds with a stoichiometric amount of the appropriate base (such as Na, Ca, Mg, or K hydroxide, carbonate, bicarbonate, or the like), or by reacting a free base form of the compound with a stoichiometric amount of the appropriate acid. Such reactions are typically carried out in water or in an organic solvent, or in a mixture of the two. Generally, non-aqueous media like ether, ethyl acetate, ethanol, isopropanol, or acetonitrile are typical, where practicable.

Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts and the quaternary ammonium salts of the parent compound formed, for example, from suitably non-toxic inorganic or organic acids. For example, conventional non-toxic acid salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, mesylic, esylic, besylic, sulfanilic, 2-acetoxybenzoic, fumaric, toluenesulfonic, methanesulfonic, ethane disulfonic, oxalic, isethionic, HOOC—(CH₂)ₙ—COOH where n is 0-4, and the like.

Additional non-limiting examples of salts include 1-hydroxy-2-naphthoic acid, 2,2-dichloroacetic acid, 2-oxoglutaric acid, 4-acetamidobenzoic acid, 4-aminosalicylic acid, adipic acid, aspartic acid, benzenesulfonic acid, camphoric acid, camphor-10-sulfonic acid, capric acid, caproic acid, caprylic acid, carbonic acid, cinnamic acid, cyclamic acid, dodecylsulfuric acid, ethane-1,2-disulfonic acid, ethanesulfonic acid, formic acid, galactaric acid, gentisic acid, glucoheptonic acid, gluconic acid, glucuronic acid, glutaric acid, glycerophosphoric acid, hippuric acid, isobutyric acid, lactobionic acid, lauric acid, malonic acid, mandelic acid, naphthalene-1,5-disulfonic acid, naphthalene-2-sulfonic acid, nicotinic acid, nitric acid, oleic acid, palmitic acid, pyroglutamic acid, sebacic acid, thiocyanic acid, and undecylenic acid. Lists of additional suitable salts may be found, e.g., in Remington's Pharmaceutical Sciences, 17^{th} ed., Mack Publishing Company, Easton, Pa., p. 1418 (1985).

The term “carrier” refers to a diluent, excipient, or vehicle with which an active compound is provided.

A “patient” or “host” or “subject” is typically a human, however, may be more generally a mammal. In an alternative embodiment it can refer to for example, a cow, sheep, goat, horses, dog, cat, rabbit, rat, mice, fish, bird and the like.

A “prodrug” as used herein, means a compound which when administered to a host in vivo is converted into a parent drug with therapeutic activity. As used herein, the term “parent drug” means the active form of the compounds that renders the biological effect to treat any of the disorders described herein, or to control or improve the underlying cause or symptoms associated with any physiological or pathological disorder described herein in a host, typically a human. Prodrugs can be used to achieve any desired effect, including to enhance properties of the parent drug or to improve the pharmaceutic or pharmacokinetic properties of the parent. Prodrug strategies exist which provide choices in modulating the conditions for in vivo generation of the parent drug, all of which are deemed included herein. Non-limiting examples of prodrug strategies include covalent attachment of removable groups, or removable portions of groups, for example, but not limited to acylation, phosphorylation, phosphonylation, phosphoramidate derivatives, amidation, reduction, oxidation, esterification, alkylation, other carboxy derivatives, sulfoxy or sulfone derivatives, carbonylation or anhydride, among others. In certain aspects of the present invention, at least one hydrophobic group is covalently bound to the parent drug to slow release of the parent drug in vivo.

A “therapeutically effective amount” of a pharmaceutical composition/combination of this invention means an amount effective, when administered to a patient, to provide a therapeutic benefit such as an amelioration of symptoms of the selected disorder, typically an ocular disorder In certain aspects, the disorder is glaucoma, a disorder mediated by carbonic anhydrase, a disorder or abnormality related to an increase in intraocular pressure (IOP), a disorder mediated by nitric oxide synthase (NOS), a disorder requiring neuroprotection such as to regenerate/repair optic nerves, allergic conjunctivitis, anterior uveitis, cataracts, dry or wet age-related macular degeneration (AMD) or diabetic retinopathy.

“y-linolenic acid” is gamma-linolenic acid.

The term “polymer” as used herein includes oligomers.

### II. Detailed Description of the Active Compounds

In certain embodiments, compounds for ocular delivery are provided that are lipophilic monoprodrugs of Furosemide, Bumetanide, Piretanide, or Ozolinone covalently linked to a biodegradable oligomer, as described in more detail herein.

In various embodiments, two biologically active compounds are covalently linked (optionally with a biodegradable linker(s), for example, that includes a linking ester, amide, etc. bond as exemplified throughout this specification in detail, e.g., —““linked through to”—) for ocular combination therapy. In some embodiments, the bis-prodrug is in a biodegradable polymeric delivery system, such as a biodegradable microparticle or nanoparticle, for controlled delivery. In one embodiment, Furosemide, Bumetanide, Piretanide, or Ozolinone is covalently linked to a β-blocker (for example, Timolol, Metipranolol, Levobunolol, Carteolol or Betaxolol). In another embodiment, Furosemide, Bumetanide, Piretanide, or Ozolinone is covalently linked to a carbonic anhydrase inhibitor (for example, Brinzolamide or Dorzolamide). In another embodiment, Furosemide, Bumetanide, Piretanide, or Ozolinone is covalently linked to an α-agonist (for example, Brimonidine or Apraclonidine). In another embodiment, Furosemide, Bumetanide, Piretanide, or Ozolinone is covalently linked to a Rho associated kinase inhibitor (for example Y-27637, AMA0076, AR-13324, RKI-1447, RKI-1313, Wf536, CID 5056270, K-115 or fasudil). In another embodiment, Furosemide, Bumetanide, Piretanide, or Ozolinone is covalently linked to a neuroprotectant DLK inhibitor (for example, Sunitinib, SR8165 axitinib, bosutinib, neratinib, Crizotinib, Tozasertib, lestautinib, foretinib or TAE-684). This invention includes the specific combination of each of the named actives with each other named active in the bis-prodrug, as if each combination were individually described (and is only written like this for efficiency of space).

In yet another embodiment, a β-blocker (for example, Timolol, Metipranolol, Levobunolol, Carteolol or Betaxolol) is covalently linked to a carbonic anhydrase inhibitor (for example, Brinzolamide or Dorzolamide). In another embodiment, a β-blocker (for example, Timolol, Metipranolol, Levobunolol, Carteolol or Betaxolol) is covalently linked to an α-agonist (for example Brimonidine or apraclonidine). In another embodiment, a β-blocker (for example, Timolol, Metipranolol, Levobunolol, Carteolol or Betaxolol) is covalently linked to a Rho associated kinase inhibitor (for example Y-27637, AMA0076, AR-13324, RKI-1447, RKI-1313, Wf536, CID 5056270, K-115 or fasudil). In another embodiment, a β-blocker (for example, Timolol, Metipranolol, Levobunolol, Carteolol or Betaxolol) is covalently linked to a neuroprotectant DLK inhibitor (for example, Sunitinib, SR8165 axitinib, bosutinib, neratinib, Crizotinib, Tozasertib, lestautinib, foretinib or TAE-684). In alternative embodiments, a ROCK inhibitor can be selected for these embodiments selected from those disclosed in Pireddu, et. Al., Pyridylthiazole-based urease as inhibitors of Rho associated protein kinases (ROCK 1 and 2), Med. Chem. Comm. 2012, 3, 699; Patel, et al., Identification of novel ROCK inhibitors with anti-migratory and anti-invasive activities, Oncogene (2014) 33, 550-555; Patel, et al, RKI-1447 is a potent inhibitor of the Rho-Associated ROCK Kinase with anti-Invasive and Antitumor Activities in Breast Cancer, Cancer Research, online Jul. 30, 2012, 5025-5033). See also U.S. Pat. Nos. 9,221,808 and 9,409,868, herein incorporated in their entirety by reference. Again, this invention includes the specific combination of each of the named actives with each other named active in the bis-prodrug, as if each combination were individually (and is only written like this for efficiency of space).

In other various embodiments, the biologically active compound as described herein for ocular therapy is covalently linked (optionally with a biodegradable linker(s) that include a linking ester, amide, etc. bond as exemplified throughout this specification in detail) to a second same biologically active compound, to create a biodegradable dimer for ocular combination therapy. The dimer is more lipophilic and thus will enhance the controlled delivery of the active compound over time, in particular in a polymeric delivery system, for example, when administered in a hydrophilic intravitreal fluid of the eye. Biologically active compounds that can be dimerized with a biodegradable linker for use in a biodegradable polymeric composition include, Furosemide, Bumetanide, Piretanide, or Ozolinone. Methods to dimerize these compounds with a biodegradable linker are exemplified throughout this specification.

According to the present invention, compounds of Formula I, Formula II, Formula III, Formula IV, Formula IV′ Formula V, Formula VI, Formula VII, Formula VIII, Formula VIII′, Formula IX, Formula X, Formula XI, Formula XII, Formula XII′, Formula XIII, Formula XIV, Formula XV, Formula XVI, Formula XVI′, Formula XVII, Formula XVIII, Formula XIX, Formula XX, Formula XX′, Formula XXI, Formula XXII, Formula XXIII, Formula XXIV, or Formula XXIV′ are provided:

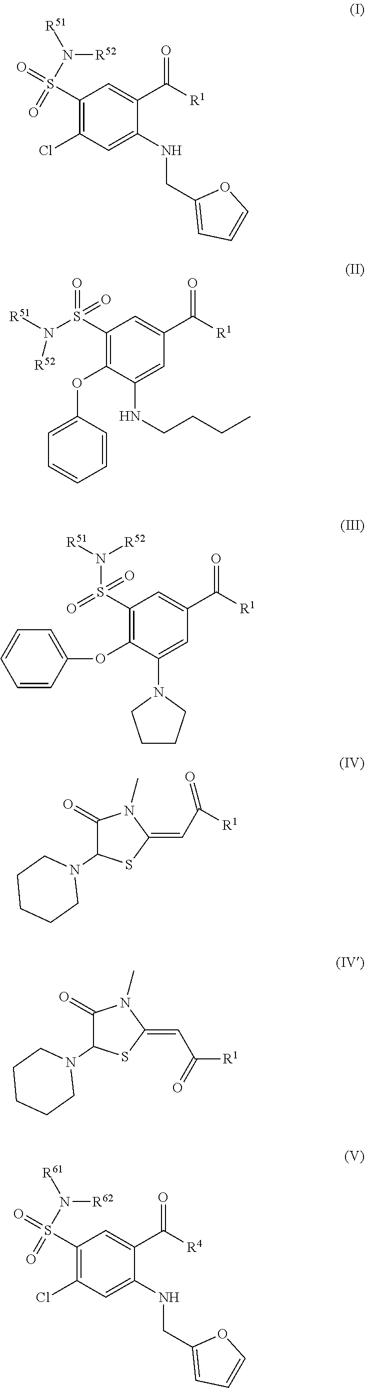

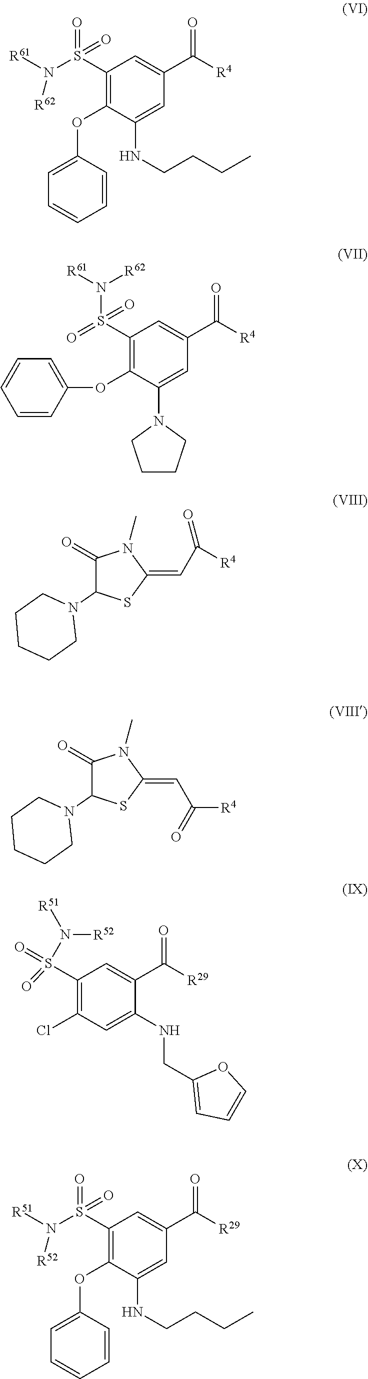

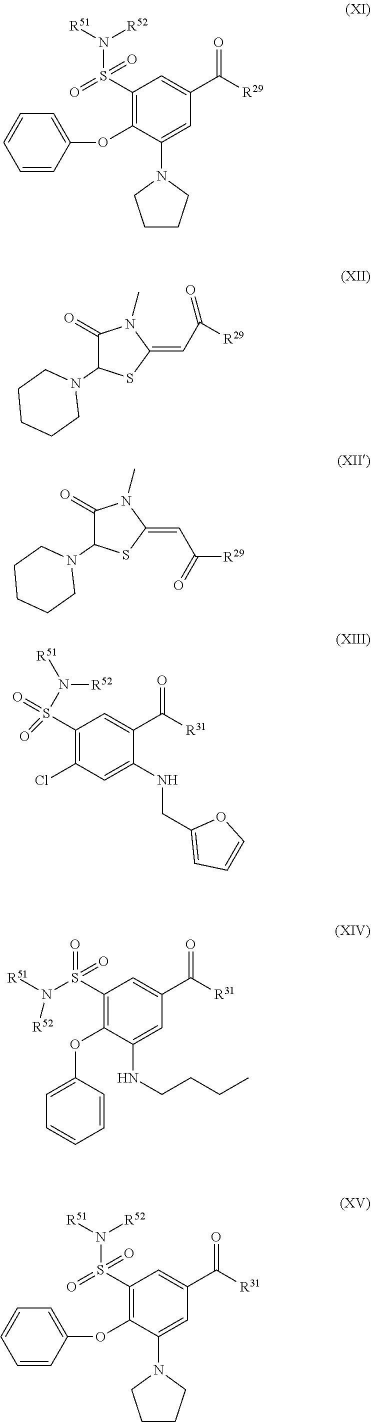

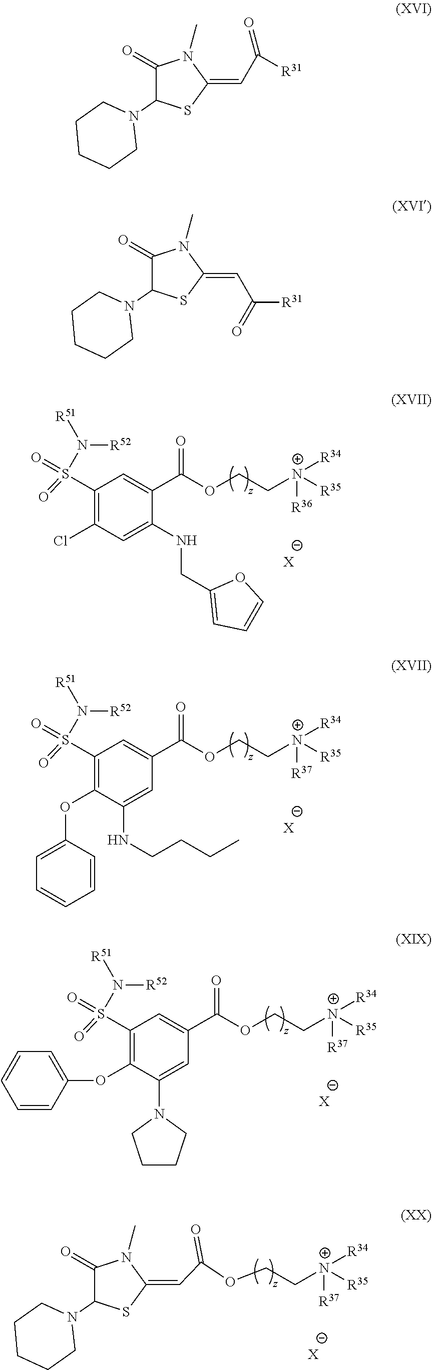

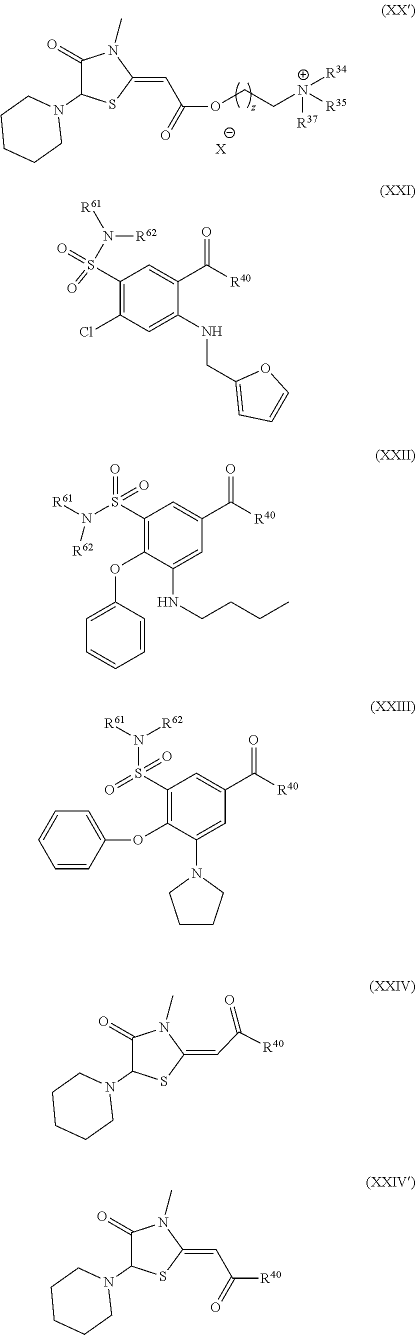

as well as the pharmaceutically acceptable salts and compositions thereof. Formula I can be considered Furosemide covalently bound to a hydrophobic moiety through an ester linkage that may be metabolized in the eye to afford Furosemide. Formula II can be considered Bumetanide covalently bound to a hydrophobic moiety through an ester linkage that may be metabolized in the eye to afford Bumetanide. Formula III can be considered Piretanide covalently bound to a hydrophobic moiety through an ester linkage that may be metabolized in the eye to afford Piretanide. Formula IV and Formula IV′ can be considered Ozolinone covalently bound to a hydrophobic moiety through an ester linkage that may be metabolized in the eye to afford Ozolinone. Formula V can be considered Furosemide covalently bound to a carbonic anhydrase inhibitor, a prostaglandin, a Rho associated kinase inhibitor, DLK inhibitor, or a β-blocker through a connecting fragment bound to both species that may be metabolized in the eye to afford both active species. Formula VI can be considered Bumetanide covalently bound to a carbonic anhydrase inhibitor, a prostaglandin, a Rho associated kinase inhibitor, DLK inhibitor, or a β-blocker through a connecting fragment bound to both species that may be metabolized in the eye to afford both active species. Formula VII can be considered Piretanide covalently bound to a carbonic anhydrase inhibitor, a prostaglandin, a Rho associated kinase inhibitor, DLK inhibitor, or a β-blocker through a connecting fragment bound to both species that may be metabolized in the eye to afford both active species. Formula VIII and Formula VIII′ can be considered Ozolinone covalently bound to a carbonic anhydrase inhibitor, a prostaglandin, a Rho associated kinase inhibitor, DLK inhibitor, or a β-blocker through a connecting fragment bound to both species that may be metabolized in the eye to afford both active species. Formula IX can be considered Furosemide covalently bound to a loop diuretic through a connecting fragment bound to both species that may be metabolized in the eye to afford both active species. Formula X can be considered Bumetanide covalently bound to a loop diuretic through a connecting fragment bound to both species that may be metabolized in the eye to afford both active species. Formula XI can be considered Piretanide covalently bound to a loop diuretic through a connecting fragment bound to both species that may be metabolized in the eye to afford both active species. Formula XII and Formula XII′ can be considered Ozolinone covalently bound to a loop diuretic through a connecting fragment bound to both species that may be metabolized in the eye to afford both active species. Formula XIII can be considered Furosemide covalently bound to a hydrophobic moiety through an ester linkage that may be metabolized in the eye to afford Furosemide. Formula XIV can be considered Bumetanide covalently bound to a hydrophobic moiety through an ester linkage that may be metabolized in the eye to afford Bumetanide. Formula XV can be considered Piretanide covalently bound to a hydrophobic moiety through an ester linkage that may be metabolized in the eye to afford Piretanide. Formula XVI and Formula XVI′ can be considered Ozolinone covalently bound to a hydrophobic moiety through an ester linkage that may be metabolized in the eye to afford Ozolinone. Formula XVII can be considered a single agent prodrug of Furosemide that may be metabolized in the eye to afford Furosemide. Formula XVIII can be considered a single agent prodrug of Bumetanide that may be metabolized in the eye to afford Bumetanide. Formula XIX can be considered a single agent prodrug of Piretanide that may be metabolized in the eye to afford Piretanide. Formula XX and Formula XX′ can be considered a single agent prodrug of Ozolinone that may be metabolized in the eye to afford Ozolinone. Formula XXI can be considered Furosemide covalently bound to a carbonic anhydrase inhibitor, a prostaglandin, a Rho associated kinase inhibitor, DLK inhibitor, or a β-blocker through a connecting fragment bound to both species that may be metabolized in the eye to afford both active species. Formula XXII can be considered Bumetanide covalently bound to a carbonic anhydrase inhibitor, a prostaglandin, a Rho associated kinase inhibitor, DLK inhibitor, or a β-blocker through a connecting fragment bound to both species that may be metabolized in the eye to afford both active species. Formula XIII can be considered Piretanide covalently bound to a carbonic anhydrase inhibitor, a prostaglandin, a Rho associated kinase inhibitor, DLK inhibitor, or a β-blocker through a connecting fragment bound to both species that may be metabolized in the eye to afford both active species. Formula XXIV and Formula XXIV′ can be considered Ozolinone covalently bound to a carbonic anhydrase inhibitor, a prostaglandin, a Rho associated kinase inhibitor, DLK inhibitor, or a β-blocker through a connecting fragment bound to both species that may be metabolized in the eye to afford both active species.

The compounds, as described herein, may include, for example, prodrugs, which are hydrolysable to form the loop diuretics Furosemide, Bumetanide, Piretanide, or Ozolinone. Furosemide was previously described in U.S. Pat. No. 3,058,882 assigned to Hoechst A G. U.S. Pat. No. 3,634,583 assigned to Lovens Kemiske Fabrik Produktionsaktieselskab describes Bumetanide and its use in pharmaceutical compositions for the treatment of oedema and hypertension. Piretanide was previously described in U.S. Pat. No. 4,118,587 assigned to Hoffmann-La Roche Inc. as a diuretic and Etozolin was previously described in U.S. Pat. No. 3,971,794 assigned to Warner-Lambery Company.

When a compound of Formula I, Formula V, Formula IX, Formula XIII, Formula XVII, or Formula XXI is administered to a mammalian subject, typically a human, the ester modification may be cleaved to release Furosemide. When a compound of Formula II, Formula VI, Formula IX, Formula XIV, Formula XVII, or Formula XXII is administered to a mammalian subject, typically a human, the ester modification may be cleaved to release Bumetanide. When a compound of Formula III, Formula VII, Formula XI, Formula XV, Formula XIX, or Formula XXIII is administered to a mammalian subject, typically a human, the ester modification may be cleaved to release Piretanide.

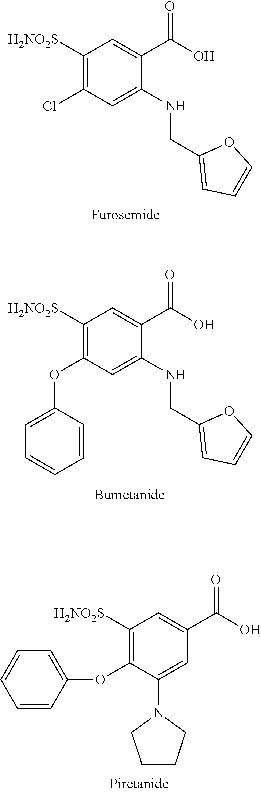 [SMILES: NS(=O)(=O)c1cc(C(=O)O)c(NCc2ccco2)cc1Cl ; NS(=O)(=O)c1cc(C(=O)O)c(NCc2ccco2)cc1Oc1ccccc1 ; NS(=O)(=O)c1cc(C(=O)O)cc(N2CCCC2)c1Oc1ccccc1]

The compounds as described herein for ocular therapy may include, for example, prodrugs, which are hydrolysable to form Ozolinone, the active metabolite of the loop diuretic Etozolin. When a compound of Formula IV, Formula VIII, Formula XII, Formula XVI, Formula XX, or Formula XXIV is administered to a mammalian subject, typically a human, the ester modification may be cleaved to release Ozolinone. When a compound of Formula IV′, Formula VIII′, Formula XII′, Formula XVI′, Formula XX′, or Formula XXIV′ is administered to a mammalian subject, typically a human, the ester modification may be cleaved to release the Z-isomer of Ozolinone.

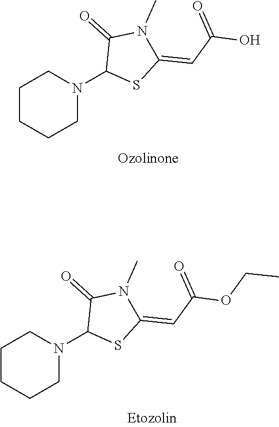 [SMILES: CN1C(=O)C(N2CCCCC2)S/C1=C/C(=O)O ; CCOC(=O)/C=C1/SC(N2CCCCC2)C(=O)N1C]

The compounds, as described herein, may include, for example, prodrugs, which are hydrolysable to form the diuretic ethacrynic acid in addition to the loop diuretics Furosemide, Bumetanide, Piretanide, or Ozolinone. Thus, when a compound of Formula IX, Formula X, Formula XI, or Formula XII is administered to a mammalian subject, typically a human, the ester modification may be cleaved to release ethacrynic acid in addition to the loop diuretics Furosemide, Bumetanide, Piretanide, or Ozolinone.

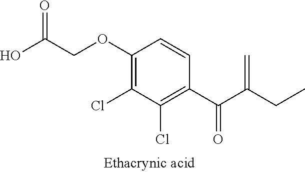 [SMILES: C=C(CC)C(=O)c1ccc(OCC(=O)O)c(Cl)c1Cl]

The compounds, as described herein, may include, for example, prodrugs, which are hydrolysable to release the active β-blocker in addition to the loop diuretics Furosemide, Bumetanide, Piretanide, or Ozolinone. Thus when a compound of Formula V, Formula VI, Formula VII, Formula VIII, Formula VIII′, Formula XXI, Formula XXII, Formula XXIII, Formula XXIV, or Formula XXIV′ is administered to a mammalian subject, typically a human, the ester bond may be cleaved to release for example Timolol, Levobunolol, Carteolol, Metipranolol, or Betaxolol in addition to the loop diuretics Furosemide, Bumetanide, Piretanide, or Ozolinone.

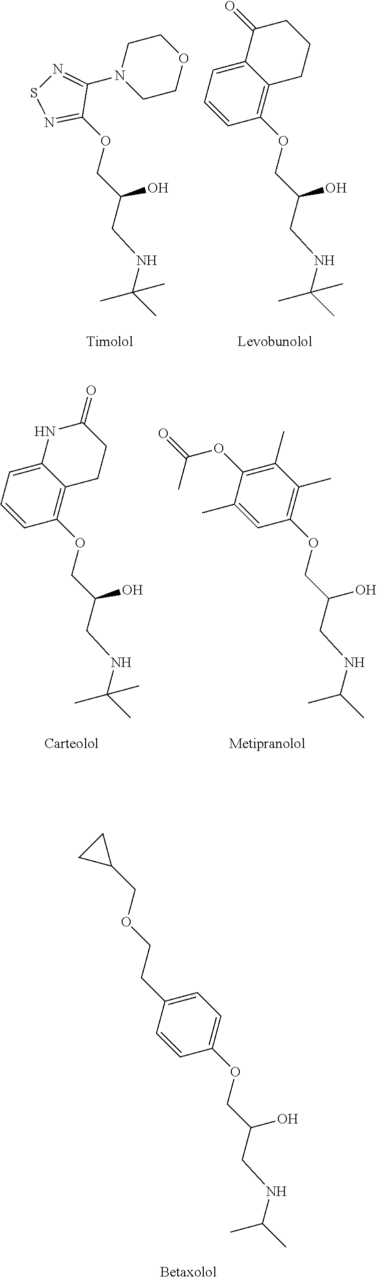

The compounds, as described herein, may include, for example, prodrugs, which are hydrolysable to form the active carboxylic acid compound shown below in addition to the loop diuretics Furosemide, Bumetanide, Piretanide, or Ozolinone. Thus, when a compound of Formula V, Formula VI, Formula VII, or Formula VIII is administered to a mammalian subject, typically a human, the ester modifications may be cleaved to release the parent free acid compound in addition to the loop diuretics Furosemide, Bumetanide, Piretanide, or Ozolinone.

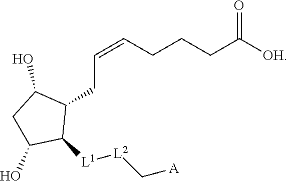

The compounds, as described herein, may include, for example, prodrugs, which are hydrolysable to form Brimonidine in addition to the loop diuretics Furosemide, Bumetanide, Piretanide, or Ozolinone. Thus when a compound of Formula V, Formula VI, Formula VII, Formula VIII′, Formula XXI, Formula XXII, Formula XXIII, Formula XXIV, or Formula XXIV′ is administered to a mammalian subject, typically a human, the amide modifications may be cleaved to release Brimonidine in addition to the loop diuretics Furosemide, Bumetanide, Piretanide, or Ozolinone.

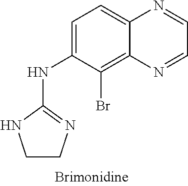 [SMILES: Brc1c(NC2=NCCN2)ccc2nccnc12]

The compounds, as described herein, may include, for example, prodrugs, which are hydrolysable to form Brinzolamide or Dorzolamide in addition to the loop diuretics Furosemide, Bumetanide, Piretanide, or Ozolinone. Thus when a compound of Formula V, Formula VI, Formula VII, Formula VIII′, Formula XXI, Formula XXII, Formula XXIII, Formula XXIV, or Formula XXIV′ is administered to a mammalian subject, typically a human, the amide modifications may be cleaved to release Brinzolamide or Dorzolamide in addition to the loop diuretics Furosemide, Bumetanide, Piretanide, or Ozolinone.

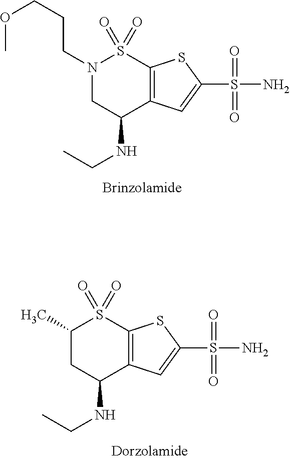 [SMILES: CCN[C@H]1CN(CCCOC)S(=O)(=O)c2sc(S(N)(=O)=O)cc21 ; CCN[C@H]1C[C@H](C)S(=O)(=O)c2sc(S(N)(=O)=O)cc21]

The compounds, as described herein, may include, for example, prodrugs, which are hydrolysable to form the active Sunitinib derivative. Thus when a compound of Formula V, Formula VI, Formula VII, or Formula VIII is administered to a mammalian subject, typically a human, the prodrug may be cleaved to release the parent Sunitinib derivative in addition to the loop diuretics Furosemide, Bumetanide, Piretanide, or Ozolinone. The active Sunitinib derivative is a phenol compound that has been demonstrated in the literature to be an active RTKI (Kuchar, M., et al. (2012). “Radioiodinated Sunitinib as a potential radiotracer for imaging angiogenesis-radiosynthesis and first radiopharmacological evaluation of 5-[125I]Iodo-Sunitinib.” Bioorg Med Chem Lett 22(8): 2850-2855. Formulations of Sunitinib for the treatment of ocular disorders and glaucoma have been described in WO2016/100392 and WO2016/100380, respectively.

The compounds, as described herein, may include, for example, prodrugs, which are hydrolysable to release a active DLK inhibitor in addition to the loop diuretics Furosemide, Bumetanide, Piretanide, or Ozolinone. Thus when a compound of Formula V, Formula VI, Formula VII, Formula VIII′, Formula XXI, Formula XXII, Formula XXIII, Formula XXIV, or Formula XXIV′ is administered to a mammalian subject, typically a human, the amide bond may be cleaved to release Crizotinib, KW-2449, a piperidino DLK inhibitor, or a Tozasertib derivative in addition to the loop diuretics Furosemide, Bumetanide, Piretanide, or Ozolinone.

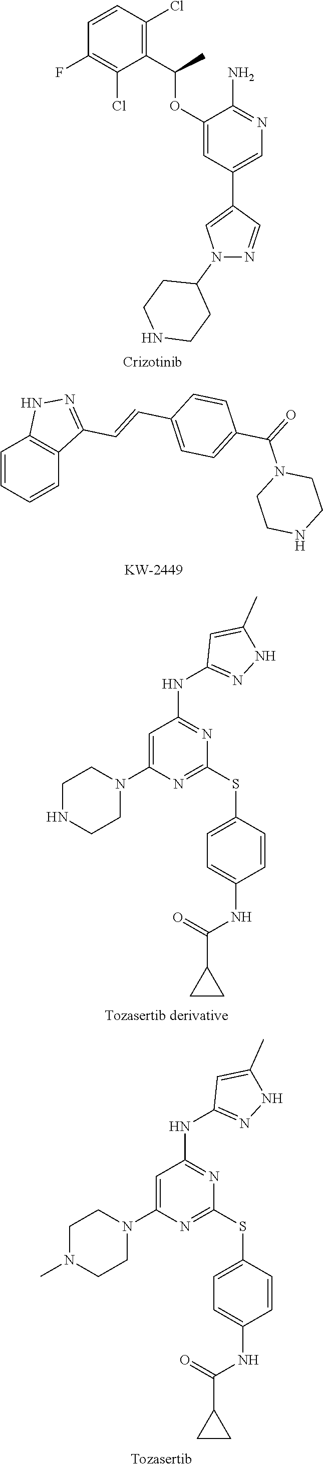 [SMILES: C[C@@H](Oc1cc(-c2cnn(C3CCNCC3)c2)cnc1N)c1c(Cl)ccc(F)c1Cl ; O=C(c1ccc(/C=C/c2n[nH]c3ccccc23)cc1)N1CCNCC1 ; Cc1cc(Nc2cc(N3CCNCC3)nc(Sc3ccc(NC(=O)C4CC4)cc3)n2)n[nH]1 ; Cc1cc(Nc2cc(N3CCN(C)CC3)nc(Sc3ccc(NC(=O)C4CC4)cc3)n2)n[nH]1]

Compounds of the present invention with stereocenters may be drawn without stereochemistry for convenience. One skilled in the art will recognize that pure enantiomers and diastereomers can be prepared by methods known in the art. Examples of methods to obtain optically active materials include at least the following.

i) Physical separation of crystals—a technique whereby macroscopic crystals of the individual enantiomers are manually separated. This technique can be used if crystals of the separate enantiomers exist, i.e., the material is a conglomerate, and the crystals are visually distinct;

ii) Simultaneous crystallization—a technique whereby the individual enantiomers are separately crystallized from a solution of the racemate, possible only if the latter is a conglomerate in the solid state;

iii) Enzymatic resolutions—a technique whereby partial or complete separation of a racemate by virtue of differing rates of reaction for the enantiomers with an enzyme;

iv) Enzymatic asymmetric synthesis—a synthetic technique whereby at least one step of the synthesis uses an enzymatic reaction to obtain an enantiomerically pure or enriched synthetic precursor of the desired enantiomer;

v) Chemical asymmetric synthesis—a synthetic technique whereby the desired enantiomer is synthesized from an achiral precursor under conditions that produce asymmetry (i.e., chirality) in the product, which may be achieved using chiral catalysts or chiral auxiliaries;

vi) Diastereomer separations—a technique whereby a racemic compound is reacted with an enantiomerically pure reagent (the chiral auxiliary) that converts the individual enantiomers to diastereomers. The resulting diastereomers are then separated by chromatography or crystallization by virtue of their now more distinct structural differences and the chiral auxiliary later removed to obtain the desired enantiomer;

vii) First- and second-order asymmetric transformations—a technique whereby diastereomers from the racemate equilibrate to yield a preponderance in solution of the diastereomer from the desired enantiomer or where preferential crystallization of the diastereomer from the desired enantiomer perturbs the equilibrium such that eventually in principle all the material is converted to the crystalline diastereomer from the desired enantiomer. The desired enantiomer is then released from the diastereomer;

viii) Kinetic resolutions—this technique refers to the achievement of partial or complete resolution of a racemate (or of a further resolution of a partially resolved compound) by virtue of unequal reaction rates of the enantiomers with a chiral, non-racemic reagent or catalyst under kinetic conditions;

ix) Enantiospecific synthesis from non-racemic precursors—a synthetic technique whereby the desired enantiomer is obtained from non-chiral starting materials and where the stereochemical integrity is not or is only minimally compromised over the course of the synthesis;

x) Chiral liquid chromatography—a technique whereby the enantiomers of a racemate are separated in a liquid mobile phase by virtue of their differing interactions with a stationary phase (including via chiral HPLC). The stationary phase can be made of chiral material or the mobile phase can contain an additional chiral material to provoke the differing interactions;

xi) Chiral gas chromatography—a technique whereby the racemate is volatilized and enantiomers are separated by virtue of their differing interactions in the gaseous mobile phase with a column containing a fixed non-racemic chiral adsorbent phase;

xii) Extraction with chiral solvents—a technique whereby the enantiomers are separated by virtue of preferential dissolution of one enantiomer into a particular chiral solvent;

xiii) Transport across chiral membranes—a technique whereby a racemate is placed in contact with a thin membrane barrier. The barrier typically separates two miscible fluids, one containing the racemate, and a driving force such as concentration or pressure differential causes preferential transport across the membrane barrier. Separation occurs as a result of the non-racemic chiral nature of the membrane that allows only one enantiomer of the racemate to pass through.

xiv) Simulated moving bed chromatography, is used in one embodiment. A wide variety of chiral stationary phases are commercially available.

I. Pharmaceutical Preparations and Formulations

One embodiment provides compositions including the compounds described herein. In certain embodiments, the composition includes a compound of Formula I, Formula II, Formula III, Formula IV, Formula IV′, Formula V, Formula VI, Formula VII, Formula VIII, Formula VIII′, Formula IX, Formula X, Formula XI, Formula XII, or Formula XII′ in combination with a pharmaceutically acceptable carrier, excipient or diluent. In certain embodiments, the composition includes a loop diuretic selected from furosemide, bumetanide, piretanide, or etozolin in combination with a pharmaceutically acceptable carrier, excipient or diluent. In one embodiment, the composition is a pharmaceutical composition for treating an eye disorder or eye disease.

Non-limiting exemplary eye disorder or disease treatable with the composition includes age related macular degeneration, alkaline erosive keratoconjunctivitis, allergic conjunctivitis, allergic keratitis, anterior uveitis, Behcet's disease, blepharitis, blood-aqueous barrier disruption, chorioiditis, chronic uveitis, conjunctivitis, contact lens-induced keratoconjunctivitis, corneal abrasion, corneal trauma, corneal ulcer, crystalline retinopathy, cystoid macular edema, dacryocystitis, diabetic keratophathy, diabetic macular edema, diabetic retinopathy, dry eye disease, dry age-related macular degeneration, geographic atrophy, eosinophilic granuloma, episcleritis, exudative macular edema, Fuchs' Dystrophy, giant cell arteritis, giant papillary conjunctivitis, glaucoma, glaucoma surgery failure, graft rejection, herpes zoster, inflammation after cataract surgery, iridocorneal endothelial syndrome, iritis, keratoconjunctiva sicca, keratoconjunctival inflammatory disease, keratoconus, lattice dystrophy, map-dot-fingerprint dystrophy, necrotic keratitis, neovascular diseases involving the retina, uveal tract or cornea, for example, neovascular glaucoma, corneal neovascularization, neovascularization resulting following a combined vitrectomy and lensectomy, neovascularization of the optic nerve, and neovascularization due to penetration of the eye or contusive ocular injury, neuroparalytic keratitis, non-infectious uveitisocular herpes, ocular lymphoma, ocular rosacea, ophthalmic infections, ophthalmic pemphigoid, optic neuritis, panuveitis, papillitis, pars planitis, persistent macular edema, phacoanaphylaxis, posterior uveitis, post-operative inflammation, proliferative diabetic retinopathy, proliferative sickle cell retinopathy, proliferative vitreoretinopathy, retinal artery occlusion, retinal detachment, retinal vein occlusion, retinitis pigmentosa, retinopathy of prematurity, rubeosis iritis, scleritis, Stevens-Johnson syndrome, sympathetic ophthalmia, temporal arteritis, thyroid associated ophthalmopathy, uveitis, vernal conjunctivitis, vitamin A insufficiency-induced keratomalacia, vitreitis, and wet age-related macular degeneration.

Non-limiting examples of methods of administration of these compositions to the eye include intravitreal, intrastromal, intracameral, sub-tenon, sub-retinal, retro-bulbar, peribulbar, suprachoroidal, choroidal, subchoroidal, conjunctival, subconjunctival, episcleral, posterior juxtascleral, circumcorneal, and tear duct injections, or through a mucus, mucin, or a mucosal barrier.

Compounds disclosed herein or used as described herein may be administered in an immediate or controlled formulation orally, topically, parenterally, by inhalation or spray, sublingually, via implant, including ocular implant, transdermally, via buccal administration, rectally, as an ophthalmic solution, injection, including ocular injection, intravenous, intra-aortal, intracranial, subdermal, intraperitoneal, systemically, subcutaneous, transnasal, sublingual, intramuscularly, intrathecal, or rectal or by other means, in dosage unit formulations containing conventional pharmaceutically acceptable carriers. For ocular delivery, the compound can be administered, as desired, for example, in an immediate or controlled formulation, as a solution, suspension, or other formulation via intravitreal, intrastromal, intracameral, sub-tenon, sub-retinal, retro-bulbar, peribulbar, suprachorodial, subchorodial, chorodial, conjunctival, subconjunctival, episcleral, periocular, transscleral, retrobulbar, posterior juxtascleral, circumcorneal, or tear duct injections, or through a mucus, mucin, or a mucosal barrier, in an immediate or controlled release fashion or via an ocular device, injection, or topically administered formulation, for example a solution or suspension provided as an eye drop.

The pharmaceutical composition may be formulated as any pharmaceutically useful form, e.g., as an aerosol, a cream, a gel, a gel cap, a pill, a microparticle, a nanoparticle, an injection or infusion solution, a capsule, a tablet, a syrup, a transdermal patch, a subcutaneous patch, a dry powder, an inhalation formulation, in a medical device, suppository, buccal, or sublingual formulation, parenteral formulation, or an ophthalmic solution or suspension. Some dosage forms, such as tablets and capsules, are subdivided into suitably sized unit doses containing appropriate quantities of the active components, e.g., an effective amount to achieve the desired purpose.

Pharmaceutical compositions, and methods of manufacturing such compositions, suitable for administration as contemplated herein are known in the art. Examples of known techniques include, for example, U.S. Pat. Nos. 4,983,593, 5,013,557, 5,456,923, 5,576,025, 5,723,269, 5,858,411, 6,254,889, 6,303,148, 6,395,302, 6,497,903, 7,060,296, 7,078,057, 7,404,828, 8,202,912, 8,257,741, 8,263,128, 8,337,899, 8,431,159, 9,028,870, 9,060,938, 9,211,261, 9,265,731, 9,358,478, and 9,387,252, incorporated by reference herein.

The pharmaceutical compositions contemplated here can optionally include a carrier. Carriers must be of sufficiently high purity and sufficiently low toxicity to render them suitable for administration to the patient being treated. The carrier can be inert or it can possess pharmaceutical benefits of its own. The amount of carrier employed in conjunction with the compound is sufficient to provide a practical quantity of material for administration per unit dose of the compound. Classes of carriers include, but are not limited to binders, buffering agents, coloring agents, diluents, disintegrants, emulsifiers, fillers, flavorants, glidents, lubricants, pH modifiers, preservatives, stabilizers, surfactants, solubilizers, tableting agents, and wetting agents. Some carriers may be listed in more than one class, for example vegetable oil may be used as a lubricant in some formulations and a diluent in others. Exemplary pharmaceutically acceptable carriers include sugars, starches, celluloses, powdered tragacanth, malt, gelatin; talc, and vegetable oils. Examples of other matrix materials, fillers, or diluents include lactose, mannitol, xylitol, microcrystalline cellulose, calcium diphosphate, and starch. Examples of surface active agents include sodium lauryl sulfate and polysorbate 80. Examples of drug complexing agents or solubilizers include the polyethylene glycols, caffeine, xanthene, gentisic acid and cylodextrins. Examples of disintegrants include sodium starch gycolate, sodium alginate, carboxymethyl cellulose sodium, methyl cellulose, colloidal silicon dioxide, and croscarmellose sodium. Examples of binders include methyl cellulose, microcrystalline cellulose, starch, and gums such as guar gum, and tragacanth. Examples of lubricants include magnesium stearate and calcium stearate. Examples of pH modifiers include acids such as citric acid, acetic acid, ascorbic acid, lactic acid, aspartic acid, succinic acid, phosphoric acid, and the like; bases such as sodium acetate, potassium acetate, calcium oxide, magnesium oxide, trisodium phosphate, sodium hydroxide, calcium hydroxide, aluminum hydroxide, and the like, and buffers generally comprising mixtures of acids and the salts of said acids. Optional other active agents may be included in a pharmaceutical composition, which do not substantially interfere with the activity of the compound of the present invention.

The pharmaceutical compositions can be formulated for oral administration. These compositions can contain any amount of active compound that achieves the desired result, for example between 0.1 and 99 weight % (wt. %) of the compound and usually at least about 5 wt. % of the compound. Some embodiments contain at least about 10%, 15%, 20%, 25 wt. % to about 50 wt. % or from about 5 wt. % to about 75 wt. % of the compound.

Pharmaceutical compositions suitable for rectal administration are typically presented as unit dose suppositories. These may be prepared by admixing the active compound with one or more conventional solid carriers, for example, cocoa butter, and then shaping the resulting mixture.

Pharmaceutical compositions suitable for topical application to the skin preferably take the form of an ointment, cream, lotion, paste, gel, spray, aerosol, or oil. Carriers which may be used include petroleum jelly, lanoline, polyethylene glycols, alcohols, transdermal enhancers, and combinations of two or more thereof.

Pharmaceutical compositions suitable for transdermal administration may be presented as discrete patches adapted to remain in intimate contact with the epidermis of the recipient for a prolonged period of time. Pharmaceutical compositions suitable for transdermal administration may also be delivered by iontophoresis (see, for example, Pharmaceutical Research 3 (6):318 (1986)) and typically take the form of an optionally buffered aqueous solution of the active compound. In one embodiment, microneedle patches or devices are provided for delivery of drugs across or into biological tissue, particularly the skin. The microneedle patches or devices permit drug delivery at clinically relevant rates across or into skin or other tissue barriers, with minimal or no damage, pain, or irritation to the tissue.

Pharmaceutical compositions suitable for administration to the lungs can be delivered by a wide range of passive breath driven and active power driven single/-multiple dose dry powder inhalers (DPI). The devices most commonly used for respiratory delivery include nebulizers, metered-dose inhalers, and dry powder inhalers. Several types of nebulizers are available, including jet nebulizers, ultrasonic nebulizers, and vibrating mesh nebulizers. Selection of a suitable lung delivery device depends on parameters, such as nature of the drug and its formulation, the site of action, and pathophysiology of the lung.

Compounds of Formula I, Formula II, Formula III, Formula IV, Formula IV′, Formula V, Formula VI, Formula VII, Formula VIII, Formula VIII′, Formula IX, Formula X, Formula XI, Formula XII, or Formula XII′ or its salt, can be delivered by any method known for ocular delivery. Methods include but are not limited to conventional or topical (solution, suspension, emulsion, ointment, inserts and gels); vesicular (liposomes, niosomes, discomes and pharmacosomes), particulates (microparticles and nanoparticles), advanced materials (scleral plugs, gene delivery, siRNA and stem cells); and controlled release systems (implants, hydrogels, dendrimers, collagen shields, polymeric solutions, therapeutic contact lenses, cyclodextrin carriers, microneedles and microemulsions).

In certain aspects, a loop diuretic selected from furosemide, bumetanide, piretanide, and etozolin is administered via intravitreal, intrastromal, intracameral, sub-tenon, sub-retinal, retro-bulbar, peribulbar, suprachoroidal, choroidal, subchoroidal, conjunctival, episcleral, posterior juxtascleral, circumcorneal, or tear duct injection in combination with one or more pharmaceutically acceptable carriers. In certain aspects, furosemide, bumetanide, or piretanide are administered in a site that is not near the trabecular meshwork. In another embodiment the selected compound is not administered topically. In certain aspects, etozolin is administered via subconjunctival injection. Representative carriers include solvents, diluents, pH modifying agents, preservatives, antioxidants, suspending agents, wetting agents, viscosity agents, tonicity agents, stabilizing agents, and combinations thereof.

The loop diuretic will preferably be formulated as a solution or suspension for injection to the eye. Pharmaceutical formulations for ocular administration are preferably in the form of a sterile aqueous solution. Acceptable solutions include, for example, water, Ringer's solution, phosphate buffered saline (PBS), and isotonic sodium chloride solution. The formulation may also be a sterile solution, suspension, or emulsion in a nontoxic, parenterally acceptable diluent or solvent such as 1,3-butanediol. In some instances, the formulation is distributed or packaged in a liquid form. Alternatively, formulations for ocular administration can be packed as a solid, obtained, for example by lyophilization of a suitable liquid formulation. The solid can be reconstituted with an appropriate carrier or diluent prior to administration.

Solutions, suspensions, ointments or emulsions for ocular administration may be buffered with an effective amount of buffer necessary to maintain a pH suitable for ocular administration. Suitable buffers are well known by those skilled in the art and some examples of useful buffers are acetate, borate, carbonate, citrate, and phosphate buffers.

Solutions, suspensions, or emulsions for ocular administration may also contain one or more tonicity agents to adjust the isotonic range of the formulation. Suitable tonicity agents are well known in the art and some examples include glycerin, mannitol, sorbitol, sodium chloride, and other electrolytes.

Solutions, suspensions, ointments or emulsions for ocular administration may also contain one or more preservatives to prevent bacterial contamination of the ophthalmic preparations. Suitable preservatives are known in the art, and include polyhexamethylenebiguanidine (PHMB), benzalkonium chloride (BAK), stabilized oxychloro complexes (otherwise known as Purite), phenylmercuric acetate, chlorobutanol, sorbic acid, chlorhexidine, benzyl alcohol, parabens, thimerosal, and mixtures thereof.

Solutions, suspensions, ointments or emulsions for ocular administration may also contain one or more excipients known art, such as dispersing agents, wetting agents, and suspending agents.

In one embodiment, the loop diuretic is administered in a dosage form that contains from about 1 μg to 10 mg, from about 1 μg to 1 mg, from about 1 μg to 100 μg, from about 1 μg to 50 μg, from about 1 μg to 10 μg, or from about 1 μg to 5 μg. In one embodiment, the loop diuretic is administered in a dosage form that contains up to about 1000, 950, 900, 850, 800, 750, 700, 650, 600, 550, 500, 450, 400, 350, 300, 250, 200, 150, 100, 90, 80, 70, 60, 50, 40, 30, 20, 15, 10, 5, or 1 μg. In another embodiment, the loop diuretic is administered in a dosage form that contains up to about 10, 9, 8, 7, 6, 5, 4, 3, 2, or 1 mg. In one embodiment, the loop diuretic is administered in a dosage form that contains at least about 1, 5, 10, 15, 20, 25, 30, 35, 40, 45, 50, 60, 70, 80, 90, 100, 150, 200, 250, 300, 350, 400, 450, 500, 550, 600, 650, 700, 750, 800, 850, 900, 950, or 1000 μg. In another embodiment, the loop diuretic is administered in a dosage form that contains at least about 1, 2, 3, 4, 5, 6, 7, 8, 9, or 10 mg.

In certain aspects, a delivery system is used including but not limited to the following; i) a degradable polymeric composition; ii) a non-degradable polymeric composition; (iii) a gel, such as a hydrogel; (iv) a depot; (v) a particle containing a core; vi) a surface-coated particle; vii) a multi-layered polymeric or non-polymeric or mixed polymeric and non-polymeric particle; viii) a polymer blend and/or ix) a particle with a coating on the surface of the particle. The polymers can include, for example, hydrophobic regions. In some embodiments, at least about 30, 40 or 50% of the hydrophobic regions in the coating molecules have a molecular mass of least about 2 kDa. In some embodiments, at least about 30, 40 or 50% of the hydrophobic regions in the coating molecules have a molecular mass of least about 3 kDa. In some embodiments, at least about 30, 40 or 50% of the hydrophobic regions in the coating molecules have a molecular mass of least about 4 kDa. In some embodiments, at least about 30, 40 or 50% of the hydrophobic regions in the coating molecules have a molecular mass of least about 5 kDa. In certain embodiments, up to 5, 10, 20, 30, 40, 50, 60, 70, 80, 90 or even 95% or more of a copolymer or polymer blend consists of a hydrophobic polymer or polymer segment. In some embodiments, the polymeric material includes up to 2, 3, 4, 5, 6, 7, 8, 9, or 10% or more hydrophilic polymer. In one embodiment, the hydrophobic polymer is a polymer or copolymer of lactic acid or glycolic acid, including PLGA. In one embodiment, the hydrophilic polymer is polyethylene glycol. In certain embodiments a triblock polymer such as a Pluronic is used. The drug delivery system can be suitable for administration into an eye compartment of a patient, for example by injection into the eye compartment. In some embodiments, the core includes a biocompatible polymer. As used herein, unless the context indicates otherwise, “drug delivery system”, “carrier”, and “particle composition” can all be used interchangeably. In a typical embodiment this delivery system is used for ocular delivery.

The particle in the drug delivery system can be of any desired size that achieves the desired result. The appropriate particle size can vary based on the method of administration, the eye compartment to which the drug delivery system is administered, the therapeutic agent employed and the eye disorder to be treated, as will be appreciated by a person of skill in the art in light of the teachings disclosed herein. For example, in some embodiments the particle has a diameter of at least about 1 nm, or from about 1 nm to about 50 microns. The particle can also have a diameter of, for example, from about 1 nm to about 15, 16, 17, 18, 19, 2, 21, 22, 23, 24, 25, 26, 27, 28, 29 or 30 microns; or from about 10 nm to about less than 30, 35, 40, 45 or 50 microns; or from about 10 nm to about less than 28 microns; from about 1 nm to about 5 microns; less than about 1 nm; from about 1 nm to about 3 microns; or from about 1 nm to about 1000 nm; or from about 25 nm to about 75 nm; or from about 20 nm to less than or about 30 nm; or from about 100 nm to about 300 nm. In some embodiments, the average particle size can be about up to 1 nm, 10 nm, 25 nm, 30 nm, 50 nm, 150 nm, 200 nm, 250 nm, 300 nm, 350 nm, 400 nm, 450 nm, 500 nm, 550 nm, 600 nm, 650 nm, 700 nm, 750 nm, 800 nm, 850 nm, 900 nm, 950 nm, 1000 nm, or more. In some embodiments, the particle size can be about 100 microns or less, about 50 microns or less, about 30 microns or less, about 10 microns or less, about 6 microns or less, about 5 microns or less, about 3 microns or less, about 1000 nm or less, about 800 nm or less, about 600 nm or less, about 500 nm or less, about 400 nm or less, about 300 nm or less, about 200 nm or less, or about 100 nm or less. In some embodiments, the particle can be a nanoparticle or a microparticle. In some embodiments, the drug delivery system can contain a plurality of sizes particles. The particles can be all nanoparticles, all microparticles, or a combination of nanoparticles and microparticles.

When delivering the active material in a polymeric delivery composition, the active material can be distributed homogeneously, heterogeneously, or in one or more polymeric layers of a multi-layered composition, including in a polymer coated core or a bare uncoated core.

In some embodiments, the drug delivery system includes a particle comprising a core. In some embodiments a loop diuretic selected from furosemide, bumetanide, piretanide, and etozolin or a compound of Formula I, Formula II, Formula III, Formula IV, Formula IV′, Formula V, Formula VI, Formula VII, Formula VIII, Formula VIII′, Formula IX, Formula X, Formula XI, Formula XII, or Formula XII′ can be present in the core in a suitable amount, e.g., at least about 1% weight (wt), at least about 5% wt, at least about 10% wt, at least about 20% wt, at least about 30% wt, at least about 40% wt, at least about 50% wt, at least about 60% wt, at least about 70% wt, at least about 80% wt, at least about 85% wt, at least about 90% wt, at least about 95% wt, or at least about 99% wt of the core. In one embodiment, the core is formed of 100% wt of the pharmaceutical agent. In some cases, the pharmaceutical agent may be present in the core at less than or equal to about 100% wt, less than or equal to about 90% wt, less than or equal to about 80% wt, less than or equal to about 70% wt, less than or equal to about 60% wt, less than or equal to about 50% wt, less than or equal to about 40% wt, less than or equal to about 30% wt, less than or equal to about 20% wt, less than or equal to about 10% wt, less than or equal to about 5% wt, less than or equal to about 2% wt, or less than or equal to about 1% wt. Combinations of the above-referenced ranges are also possible (e.g., present in an amount of at least about 80% wt and less than or equal to about 100% wt). Other ranges are also possible.

In embodiments in which the core particles comprise relatively high amounts of a pharmaceutical agent (e.g., at least about 50% wt of the core particle), the core particles generally have an increased loading of the pharmaceutical agent compared to particles that are formed by encapsulating agents into polymeric carriers. This is an advantage for drug delivery applications, since higher drug loadings mean that fewer numbers of particles may be needed to achieve a desired effect compared to the use of particles containing polymeric carriers.

In some embodiments, the core is formed of a solid material having a relatively low aqueous solubility (i.e., a solubility in water, optionally with one or more buffers), and/or a relatively low solubility in the solution in which the solid material is being coated with a surface-altering agent. For example, the solid material may have an aqueous solubility (or a solubility in a coating solution) of less than or equal to about 5 mg/mL, less than or equal to about 2 mg/mL, less than or equal to about 1 mg/mL, less than or equal to about 0.5 mg/mL, less than or equal to about 0.1 mg/mL, less than or equal to about 0.05 mg/mL, less than or equal to about 0.01 mg/mL, less than or equal to about 1 μg/mL, less than or equal to about 0.1 μg/mL, less than or equal to about 0.01 μg/mL, less than or equal to about 1 ng/mL, less than or equal to about 0.1 ng/mL, or less than or equal to about 0.01 ng/mL at 25° C. In some embodiments, the solid material may have an aqueous solubility (or a solubility in a coating solution) of at least about 1 pg/mL, at least about 10 pg/mL, at least about 0.1 ng/mL, at least about 1 ng/mL, at least about 10 ng/mL, at least about 0.1 μg/mL, at least about 1 μg/mL, at least about 5 μg/mL, at least about 0.01 mg/mL, at least about 0.05 mg/mL, at least about 0.1 mg/mL, at least about 0.5 mg/mL, at least about 1.0 mg/mL, at least about 2 mg/mL. Combinations of the above-noted ranges are possible (e.g., an aqueous solubility or a solubility in a coating solution of at least about 10 pg/mL and less than or equal to about 1 mg/mL). Other ranges are also possible. The solid material may have these or other ranges of aqueous solubilities at any point throughout the pH range (e.g., from pH 1 to pH 14).

In some embodiments, the core may be formed of a material within one of the ranges of solubilities classified by the U.S. Pharmacopeia Convention: e.g., very soluble: >1,000 mg/mL; freely soluble: 100-1,000 mg/mL; soluble: 33-100 mg/mL; sparingly soluble: 10-33 mg/mL; slightly soluble: 1-10 mg/mL; very slightly soluble: 0.1-1 mg/mL; and practically insoluble: <0.1 mg/mL.

Although a core may be hydrophobic or hydrophilic, in many embodiments described herein, the core is substantially hydrophobic. “Hydrophobic” and “hydrophilic” are given their ordinary meaning in the art and, as will be understood by those skilled in the art, in many instances herein, are relative terms. Relative hydrophobicities and hydrophilicities of materials can be determined by measuring the contact angle of a water droplet on a planar surface of the substance to be measured, e.g., using an instrument such as a contact angle goniometer and a packed powder of the core material.

In some embodiments, the core particles described herein may be produced by nanomilling of a solid material (e.g., a compound of Formula I, Formula II, Formula III, Formula IV, Formula IV′, Formula V, Formula VI, Formula VII, Formula VIII, Formula VIII′, Formula IX, Formula X, Formula XI, Formula XII, or Formula XII′) in the presence of one or more stabilizers/surface-altering agents. Small particles of a solid material may require the presence of one or more stabilizers/surface-altering agents, particularly on the surface of the particles, in order to stabilize a suspension of particles without agglomeration or aggregation in a liquid solution. In some such embodiments, the stabilizer may act as a surface-altering agent, forming a coating on the particle.

In a wet milling process, milling can be performed in a dispersion (e.g., an aqueous dispersion) containing one or more stabilizers (e.g., a surface-altering agent), a grinding medium, a solid to be milled (e.g., a solid pharmaceutical agent), and a solvent. Any suitable amount of a stabilizer/surface-altering agent can be included in the solvent. In some embodiments, a stabilizer/surface-altering agent may be present in the solvent in an amount of at least about 0.001% (wt or % weight to volume (w:v)), at least about 0.01, at least about 0.1, at least about 0.5, at least about 1, at least about 2, at least about 3, at least about 4, at least about 5, at least about 6, at least about 7, at least about 8, at least about 10, at least about 12, at least about 15, at least about 20, at least about 40, at least about 60, or at least about 80% of the solvent. In some cases, the stabilizer may be present in the solvent in an amount of about 100% (e.g., in an instance where the stabilizer/surface-altering agent is the solvent). In other embodiments, the stabilizer may be present in the solvent in an amount of less than or equal to about 100, less than or equal to about 80, less than or equal to about 60, less than or equal to about 40, less than or equal to about 20, less than or equal to about 15, less than or equal to about 12, less than or equal to about 10, less than or equal to about 8, less than or equal to about 7%, less than or equal to about 6%, less than or equal to about 5%, less than or equal to about 4%, less than or equal to about 3%, less than or equal to about 2%, or less than or equal to about 1% of the solvent. Combinations of the above-referenced ranges are also possible (e.g., an amount of less than or equal to about 5% and at least about 1% of the solvent). Other ranges are also possible. The particular range chosen may influence factors that may affect the ability of the particles to penetrate mucus such as the stability of the coating of the stabilizer/surface-altering agent on the particle surface, the average thickness of the coating of the stabilizer/surface-altering agent on the particles, the orientation of the stabilizer/surface-altering agent on the particles, the density of the stabilizer/surface altering agent on the particles, stabilizer/drug ratio, drug concentration, the size and polydispersity of the particles formed, and the morphology of the particles formed.

The compound of Formula I, Formula II, Formula III, Formula IV, Formula IV′, Formula V, Formula VI, Formula VII, Formula VIII, Formula VIII′, Formula IX, Formula X, Formula XI, Formula XII, or Formula XII′ or a loop diuretic selected from furosemide, bumetanide, piretanide, and etozolin (or salt thereof) may be present in the solvent in any suitable amount. In some embodiments, the pharmaceutical agent (or salt thereof) is present in an amount of at least about 0.001% (wt % or % weight to volume (w:v)), at least about 0.01%, at least about 0.1%, at least about 0.5%, at least about 1%, at least about 2%, at least about 3%, at least about 4%, at least about 5%, at least about 6%, at least about 7%, at least about 8%, at least about 10%, at least about 12%, at least about 15%, at least about 20%, at least about 40%, at least about 60%, or at least about 80% of the solvent. In some cases, the pharmaceutical agent (or salt thereof) may be present in the solvent in an amount of less than or equal to about 100%, less than or equal to about 90%, less than or equal to about 80%, less than or equal to about 60%, less than or equal to about 40%, less than or equal to about 20%, less than or equal to about 15%, less than or equal to about 12%, less than or equal to about 10%, less than or equal to about 8%, less than or equal to about 7%, less than or equal to about 6%, less than or equal to about 5%, less than or equal to about 4%, less than or equal to about 3%, less than or equal to about 2%, or less than or equal to about 1% of the solvent. Combinations of the above-referenced ranges are also possible (e.g., an amount of less than or equal to about 20% and at least about 1% of the solvent). In some embodiments, the pharmaceutical agent is present in the above ranges but in w:v.

The ratio of stabilizer/surface-altering agent to pharmaceutical agent (or salt thereof) in a solvent may also vary. In some embodiments, the ratio of stabilizer/surface-altering agent to pharmaceutical agent (or salt thereof) may be at least 0.001:1 (weight ratio, molar ratio, or w:v ratio), at least 0.01:1, at least 0.01:1, at least 1:1, at least 2:1, at least 3:1, at least 5:1, at least 10:1, at least 25:1, at least 50:1, at least 100:1, or at least 500:1. In some cases, the ratio of stabilizer/surface-altering agent to pharmaceutical agent (or salt thereof) may be less than or equal to 1000:1 (weight ratio or molar ratio), less than or equal to 500:1, less than or equal to 100:1, less than or equal to 75:1, less than or equal to 50:1, less than or equal to 25:1, less than or equal to 10:1, less than or equal to 5:1, less than or equal to 3:1, less than or equal to 2:1, less than or equal to 1:1, or less than or equal to 0.1:1.

Combinations of the above-referenced ranges are possible (e.g., a ratio of at least 5:1 and less than or equal to 50:1). Other ranges are also possible.

Stabilizers/surface-altering agents may be, for example, polymers or surfactants. Examples of polymers are those suitable for use in coatings, as described in more detail below. Non-limiting examples of surfactants include L-a-phosphatidylcholine (PC), 1,2-dipalmitoylphosphatidycholine (DPPC), oleic acid, sorbitan trioleate, sorbitan mono-oleate, sorbitan monolaurate, polyoxyethylene sorbitan monolaurate, polyoxyethylene sorbitan monooleate, natural lecithin, oleyl polyoxyethylene ether, stearyl polyoxyethylene ether, lauryl polyoxyethylene ether, block copolymers of oxyethylene and oxypropylene, synthetic lecithin, diethylene glycol dioleate, tetrahydrofurfuryl oleate, ethyl oleate, isopropyl myristate, glyceryl monooleate, glyceryl monostearate, glyceryl monoricinoleate, cetyl alcohol, stearyl alcohol, polyethylene glycol 400, cetyl pyridinium chloride, benzalkonium chloride, olive oil, glyceryl monolaurate, corn oil, cotton seed oil, and sunflower seed oil. Derivatives of the above-noted compounds are also possible. Combinations of the above-noted compounds and others described herein may also be used as surface-altering agents in the inventive particles. As described herein, in some embodiments a surface-altering agent may act as a stabilizer, a surfactant, and/or an emulsifier. In some embodiments, the surface altering agent may aid particle transport in mucus.

It should be appreciated that while in some embodiments the stabilizer used for milling forms a coating on a particle surface, which coating renders particle mucus penetrating, in other embodiments, the stabilizer may be exchanged with one or more other surface-altering agents after the particle has been formed. For example, in one set of methods, a first stabilizer/surface-altering agent may be used during a milling process and may coat a surface of a core particle, and then all or portions of the first stabilizer/surface-altering agent may be exchanged with a second stabilizer/surface-altering agent to coat all or portions of the core particle surface. In some cases, the second stabilizer/surface-altering agent may render the particle mucus penetrating more than the first stabilizer/surface-altering agent. In some embodiments, a core particle having a coating including multiple surface-altering agents may be formed.

In other embodiments, core particles may be formed by a precipitation technique. Precipitation techniques (e.g., microprecipitation techniques, nanoprecipitation techniques) may involve forming a first solution comprising a compound of Formula I, Formula II, Formula III, Formula IV, Formula IV′, Formula V, Formula VI, Formula VII, Formula VIII, Formula VIII′, Formula IX, Formula X, Formula XI, Formula XII, or Formula XII′ or a loop diuretic selected from furosemide, bumetanide, piretanide, and etozolin and a solvent, wherein the material is substantially soluble in the solvent. The solution may be added to a second solution comprising another solvent in which the material is substantially insoluble, thereby forming a plurality of particles comprising the material. In some cases, one or more surface-altering agents, surfactants, materials, and/or bioactive agents may be present in the first and/or second solutions. A coating may be formed during the process of precipitating the core (e.g., the precipitating and coating steps may be performed substantially simultaneously). In other embodiments, the particles are first formed using a precipitation technique, following by coating of the particles with a surface-altering agent.

In some embodiments, a precipitation technique may be used to form particles (e.g., nanocrystals) of a salt of a compound of Formula I, Formula II, Formula III, Formula IV, Formula IV′, Formula V, Formula VI, Formula VII, Formula VIII, Formula VIII′, Formula IX, Formula X, Formula XI, Formula XII, or Formula XII′ or a loop diuretic selected from furosemide, bumetanide, piretanide, and etozolin. Generally, a precipitation technique involves dissolving the material to be used as the core in a solvent, which is then added to a miscible anti-solvent with or without excipients to form the core particle. This technique may be useful for preparing particles of pharmaceutical agents that are soluble in aqueous solutions (e.g., agents having a relatively high aqueous solubility). In some embodiments, pharmaceutical agents having one or more charged or ionizable groups can interact with a counter ion (e.g., a cation or an anion) to form a salt complex.

As described herein, in some embodiments, a method of forming a core particle involves choosing a stabilizer that is suitable for both nanomilling and for forming a coating on the particle and rendering the particle mucus penetrating. For example, as described in more detail below, it has been demonstrated that 200-500 nm nanoparticles of a model compound pyrene produced by nanomilling of pyrene in the presence of Pluronic® F127 resulted in particles that can penetrate physiological mucus samples at the same rate as well-established polymer-based MPP. Interestingly, it was observed that only a handful of stabilizers/surface-altering agents tested fit the criteria of being suitable for both nanomilling and for forming a coating on the particle that renders the particle mucus penetrating, as described in more detail below.

II. Description of Polymeric Delivery Materials

The particles of the drug delivery system can include a biocompatible polymer. As used herein, the term “biocompatible polymer” encompasses any polymer than can be administered to a patient without an unacceptable adverse effect to the patient.

Examples of biocompatible polymers include but are not limited to polystyrenes; poly(112ydroxyl acid); poly(lactic acid); poly(glycolic acid); poly(lactic acid-co-glycolic acid); poly(lactic-co-glycolic acid); poly(lactide); poly(glycolide); poly(lactide-co-glycolide); polyanhydrides; polyorthoesters; polyamides; polycarbonates; polyalkylenes; polyethylenes; polypropylene; polyalkylene glycols; poly(ethylene glycol); polyalkylene oxides; poly(ethylene oxides); polyalkylene terephthalates; poly(ethylene terephthalate); polyvinyl alcohols; polyvinyl ethers; polyvinyl esters; polyvinyl halides; poly(vinyl chloride); polyvinylpyrrolidone; polysiloxanes; poly(vinyl alcohols); poly(vinyl acetate); polyurethanes; co-polymers of polyurethanes; derivativized celluloses; alkyl cellulose; hydroxyalkyl celluloses; cellulose ethers; cellulose esters; nitro celluloses; methyl cellulose; ethyl cellulose; hydroxypropyl cellulose; 112ydroxyl-propyl methyl cellulose; hydroxybutyl methyl cellulose; cellulose acetate; cellulose propionate; cellulose acetate butyrate; cellulose acetate phthalate; carboxylethyl cellulose; cellulose triacetate; cellulose sulfate sodium salt; polymers of acrylic acid; methacrylic acid; copolymers of methacrylic acid; derivatives of methacrylic acid; poly(methyl methacrylate); poly(ethyl methacrylate); poly(butylmethacrylate); poly(isobutyl methacrylate); poly(hexylmethacrylate); poly(isodecyl methacrylate); poly(lauryl methacrylate); poly(phenyl methacrylate); poly(methyl acrylate); poly(isopropyl acrylate); poly(isobutyl acrylate); poly(octadecyl acrylate); poly(butyric acid); poly(valeric acid); poly(lactide-co-caprolactone); copolymers of poly(lactide-co-caprolactone); blends of poly(lactide-co-caprolactone); hydroxyethyl methacrylate (HEMA); copolymers of HEMA with acrylate; copolymers of HEMA with polymethylmethacrylate (PMMA); polyvinylpyrrolidone/vinyl acetate copolymer (PVP/VA); acrylate polymers/copolymers; acrylate/carboxyl polymers; acrylate hydroxyl and/or carboxyl copolymers; polycarbonate-urethane polymers; silicone-urethane polymers; epoxy polymers; cellulose nitrates; polytetramethylene ether glycol urethane; polymethylmethacrylate-2-hydroxyethylmethacrylate copolymer; polyethylmethacrylate-2-hydroxyethylmethacrylate copolymer; polypropylmethacrylate-2-hydroxyethylmethacrylate copolymer; polybutylmethacrylate-2-hydroxyethylmethacrylate copolymer; polymethylacrylate-2-hydroxyethylmethacrylate copolymer; polyethylacrylate-2-hydroxyethylmethacrylate copolymer; polypropylacrylate-2-hydroxymethacrylate copolymer; polybutylacrylate-2-hydroxyethylmethacrylate copolymer; copolymermethylvinylether maleicanhydride copolymer; poly (2-hydroxyethyl methacrylate) polymer/copolymer; acrylate carboxyl and/or 113ydroxyl copolymer; olefin acrylic acid copolymer; ethylene acrylic acid copolymer; polyamide polymers/copolymers; polyimide polymers/copolymers; ethylene vinylacetate copolymer; polycarbonate urethane; silicone urethane; polyvinylpyridine copolymers; polyether sulfones; polygalactin, poly-(isobutyl cyanoacrylate), and poly(2-hydroxyethyl-L-glutamine); polydimethyl siloxane; poly(caprolactones); poly(ortho esters); polyamines; polyethers; polyesters; polycarbamates; polyureas; polyimides; polysulfones; polyacetylenes; polyethyeneimines; polyisocyanates; polyacrylates; polymethacrylates; polyacrylonitriles; polyarylates; and combinations, copolymers and/or mixtures of two or more of any of the foregoing. In some cases, the particle includes a hydrophobic material and at least one bioactive agent. In certain embodiments, the hydrophobic material is used instead of a polymer. In other embodiments, the hydrophobic material is used in addition to a polymer.

An active compound as described herein can be physically mixed in the polymeric material, including in an interpenetrating polymer network or can be covalently bound to the polymeric material

Linear, non-linear or linear multiblock polymers or copolymers can be used to form nanoparticles, microparticles, and implants (e.g., rods, discs, wafers, etc.) useful for the delivery to the eye. The polymers can contain one or more hydrophobic polymer segments and one or more hydrophilic polymer segments covalently connected through a linear link or multivalent branch point to form a non-linear multiblock copolymer containing at least three polymeric segments. The polymer can be a conjugate further containing one or more therapeutic, prophylactic, or diagnostic agents covalently attached to the one or more polymer segments. By employing a polymer-drug conjugate, particles can be formed with more controlled drug loading and drug release profiles. In addition, the solubility of the conjugate can be controlled so as to minimize soluble drug concentration and, therefore, toxicity.

The one or more hydrophobic polymer segments, independently, can be any biocompatible hydrophobic polymer or copolymer. In some cases, the one or more hydrophobic polymer segments are also biodegradable. Examples of suitable hydrophobic polymers include polyesters such as polylactic acid, polyglycolic acid, or polycaprolactone, polyanhydrides, such as polysebacic anhydride, and copolymers thereof. In certain embodiments, the hydrophobic polymer is a polyanhydride, such as polysebacic anhydride or a copolymer thereof. The one or more hydrophilic polymer segments can be any hydrophilic, biocompatible, suitably non-toxic polymer or copolymer. The hydrophilic polymer segment can be, for example, a poly(alkylene glycol), a polysaccharide, poly(vinyl alcohol), polypyrrolidone, a polyoxyethylene block copolymer (PLURONIC) or a copolymers thereof. In preferred embodiments, the one or more hydrophilic polymer segments are, or are composed of, polyethylene glycol (PEG).

WO 2016/100380A1 and WO 2016/100392 A1 describe certain Sunitinib delivery systems, which can also be used in the present invention to deliver the IOP lowering agents provided by the current invention, and as described further herein. For example, a process similar to that used in WO 2016/100380A1 and WO 2016/100392 A1 to prepare a polymeric Sunitinib drug formulation can be utilized: (i) dissolve or disperse the IOP lowering agent or its salt in an organic solvent; (ii) mix the solution/dispersion of step (i) with a polymer solution that has a viscosity of at least about 300 cPs (or perhaps at least about 350, 400, 500, 600, 700 or 800 or more cPs); (iii) mix the drug polymer solution/dispersion of step (ii) with an aqueous solution optionally with a surfactant or emulsifier, to form a solvent-laden encapsulated microparticle; and (iv) isolate the microparticles. Drug loading is also significantly affected by the method of making and the solvent used. For example, S/O/W single emulsion method will yield a higher loading than O/W single emulsion method even without control the acid value. In addition, W/O/W double emulsions have been shown to significantly improve drug loading of less hydrophobic salt forms over single O/W emulsions. The ratio of continuous phase to dispersed phase can also significantly alter the encapsulation efficiency and drug loading by modulation of the rate of particle solidification. The rate of polymer solidification with the evaporation of solvent affects the degree of porosity within microparticles. A large CP:DP ratio results in faster polymer precipitation, less porosity, and higher encapsulation efficiency and drug loading. However, decreasing the rate of evaporation of the solvent during particle preparation can also lead to improvements in drug loading of highly polar compounds. As the organic phase evaporates, highly polar compounds within the organic phase is driven to the surface of the particles resulting in poor encapsulation and drug loading. By decreasing the rate of solvent evaporation by decreasing the temperature or rate of stirring, encapsulation efficiency and % drug loading can be increased for highly polar compounds. These technologies can be used by one of skill in the art to deliver any of the active compounds as described generally in this specification.

U.S. Pat. No. 8,889,193 and PCT/US2011/026321 disclose, for example, a method for treating an eye disorder in a patient in need thereof, comprising administering into the eye, for example, by intravitreal injection into the vitreous chamber of the eye, an effective amount of a drug delivery system which comprises: (i) a microparticle including a core which includes the biodegradable polymer polylactide-co-glycolide; (ii) a coating associated with the core which is non-covalently associated with the microparticle particle; wherein the coating molecule has a hydrophilic region and a hydrophobic region, and wherein the hydrophilic region is polyethylene glycol; and (iii) a therapeutically effective amount of a therapeutic agent, wherein the drug delivery system provides sustained release of the therapeutic agent into the vitreous chamber over a period of time of at least three months; and wherein the vitreous chamber of the eye exhibits at least 10% less inflammation or intraocular pressure than if the particle were uncoated. In certain embodiments, the microparticle can be about 50 or 30 microns or less. The delivery system described in U.S. Pat. No. 8,889,193 and PCT/US2011/026321 can be used to deliver any of the active agents described herein.

In some embodiments, the drug delivery systems contain a particle with a coating on the surface, wherein the coating molecules have hydrophilic regions and, optionally, hydrophobic regions,

The drug delivery system can include a coating. The coating can be disposed on the surface of the particle, for example by bonding, adsorption or by complexation. The coating can also be intermingled or dispersed within the particle as well as disposed on the surface of the particle.

The homogeneous or heterogenous polymer or polymeric coating can be, for example, polyethylene glycol, polyvinyl alcohol (PVA), or similar substances. The coating can be, for example, vitamin E-PEG 1k or vitamin E-PEG 5k or the like. Vitamin E-PEG 5k can help present a dense coating of PEG on the surface of a particle. The coating can also include nonionic surfactants such as those composed of polyalkylene oxide, e.g., polyoxyethylene (PEO), also referred to herein as polyethylene glycol; or polyoxypropylene (PPO), also referred to herein as polypropylene glycol (PPG), and can include a copolymer of more than one alkylene oxide.

The polymer or copolymer can be, for example, a random copolymer, an alternating copolymer, a block copolymer or graft copolymer.

In some embodiments, the coating can include a polyoxyethylene-polyoxypropylene copolymer, e.g., block copolymer of ethylene oxide and propylene oxide. (i.e., poloxamers). Examples of poloxamers suitable for use in the present invention include, for example, poloxamers 188, 237, 338 and 407. These poloxamers are available under the trade name Pluronic® (available from BASF, Mount Olive, N.J.) and correspond to Pluronic® F-68, F-87, F-108 and F-127, respectively. Poloxamer 188 (corresponding to Pluronic® F-68) is a block copolymer with an average molecular mass of about 7,000 to about 10,000 Da, or about 8,000 to about 9,000 Da, or about 8,400 Da. Poloxamer 237 (corresponding to Pluronic® F-87) is a block copolymer with an average molecular mass of about 6,000 to about 9,000 Da, or about 6,500 to about 8,000 Da, or about 7,7000 Da. Poloxamer 338 (corresponding to Pluronic® F-108) is a block copolymer with an average molecular mass of about 12,000 to about 18,000 Da, or about 13,000 to about 15,000 Da, or about 14,600 Da. Poloxamer 407 (corresponding to Pluronic® F-127) is a polyoxyethylene-polyoxypropylene triblock copolymer in a ratio of between about E₁₀₁ P₅₆E₁₀₁ to about E₁₀₆P₇₀E₁₀₆, or about E₁₀₁ P₅₆E₁₀₁, or about E₁₀₆P₇₀E₁₀₆, with an average molecular mass of about 10,000 to about 15,000 Da, or about 12,000 to about 14,000 Da, or about 12,000 to about 13,000 Da, or about 12,600 Da. For example, the NF forms of poloxamers or Pluronic® polymers can be used.

In some embodiments, the polymer can be, for example Pluronic® P103 or Pluronic® P105. Pluronic® P103 is a block copolymer with an average molecular mass of about 3,000 Da to about 6,000 Da, or about 4,000 Da to about 6,000 Da, or about 4,950 Da. Pluronic® P105 is a block copolymer with an average molecular mass of about 5,000 Da to about 8,000 Da, or about 6,000 Da to about 7,000 Da, or about 6,500 Da.

In some embodiments, the polymer can have an average molecular weight of about 9,000 Da or greater, about 10,000 Da or greater, about 11,000 Da or greater or about 12,000 Da or greater. In exemplary embodiments, the polymer can have an average molecular weight of from about 10,000 to about 15,000 Da, or about 12,000 to about 14,000 Da, or about 12,000 to about 13,000 Da, or about 12,600 Da. In some embodiments, the polymer can be selected from Pluronic® P103, P105, F-68, F-87, F-108 and F-127, from Pluronic® P103, P105, F-87, F-108 and F-127, or from Pluronic® P103, P105, F-108 and F-127, or from Pluronic® P103, P105 and F-127. In some embodiments, the polymer can be Pluronic® F-127. In representative embodiments, the polymer is associated with the particles. For example, the polymer can be covalently attached to the particles. In representative embodiments, the polymer comprises polyethylene glycol, which is covalently attached to a selected polymer, yielding what is commonly referred to as a PEGylated particle.

In some embodiments, a coating is non-covalently associated with a core particle. This association can be held together by any force or mechanism of molecular interaction that permits two substances to remain in substantially the same positions relative to each other, including intermolecular forces, dipole-dipole interactions, van der Waals forces, hydrophobic interactions, electrostatic interactions and the like. In some embodiments, the coating is adsorbed onto the particle. According to representative embodiments, a non-covalently bound coating can be comprised of portions or segments that promote association with the particle, for example by electrostatic or van der Waals forces. In some embodiments, the interaction is between a hydrophobic portion of the coating and the particle. Embodiments include particle coating combinations which, however attached to the particle, present a hydrophilic region, e.g. a PEG rich region, to the environment around the particle coating combination. The particle coating combination can provide both a hydrophilic surface and an uncharged or substantially neutrally-charged surface, which can be biologically inert.

Suitable polymers for use according to the compositions and methods disclosed herein can be made up of molecules having hydrophobic regions as well as hydrophilic regions. Without wishing to be bound by any particular theory, when used as a coating, it is believed that the hydrophobic regions of the molecules are able to form adsorptive interactions with the surface of the particle, and thus maintain a non-covalent association with it, while the hydrophilic regions orient toward the surrounding, frequently aqueous, environment. In some embodiments the hydrophilic regions are characterized in that they avoid or minimize adhesive interactions with substances in the surrounding environment. Suitable hydrophobic regions in a coatings can include, for example, PPO, vitamin E and the like, either alone or in combination with each other or with other substances. Suitable hydrophilic regions in the coatings can include, for example, PEG, heparin, polymers that form hydrogels and the like, alone or in combination with each other or with other substances.

Representative coatings according to the compositions and methods disclosed herein can include molecules having, for example, hydrophobic segments such as PPO segments with molecular weights of at least about 1.8 kDa, or at least about 2 kDa, or at least about 2.4 kDa, or at least about 2.8 kDa, or at least about 3.2 kDa, or at least about 3.6 kDa, or at least about 4.0 kDa, or at least about 4.4 kDa, or at least about 4.8 kDa or at least about 5.2 kDa, or at least 5.6 kDa, or at least 6.0 kDa, or at least 6.4 kDa or more. In some embodiments, the coatings can have PPO segments with molecular weights of from about 1.8 kDa to about 10 kDa, or from about 2 kDa to about 5 kDa, or from about 2.5 kDa to about 4.5 kDa, or from about 2.5 kDa to about 3.5 kDa, or from about 3 kDa to about 6 kDa, or from about 3 kDa to about 5 kDa, or from about 4 kDa to about 6 kDa, or from about 4 kDa to about 7 kDa. In some embodiments, at least about 10%, or at least about 25%, or at least about 50%, or at least about 75%, or at least about 90%, or at least about 95%, or at least about 99% or more of the hydrophobic regions in these coatings have molecular weights within these ranges. In some embodiments, the coatings are biologically inert. Compounds that generate both a hydrophilic surface and an uncharged or substantially neutrally-charged surface can be biologically inert.

Representative coatings according to the compositions and methods disclosed herein can include molecules having, for example, hydrophobic segments such as PEG segments with molecular weights of at least about 1.8 kDa, or at least about 2 kDa, or at least about 2.4 kDa, or at least about 2.8 kDa, or at least about 3.2 kDa, or at least about 3.6 kDa, or at least about 4.0 kDa, or at least about 4.4 kDa, or at least about 4.8 kDa, or at least about 5.2 kDa, or at least 5.6 kDa, or at least 6.0 kDa, or at least 6.4 kDa or more. In some embodiments, the coatings can have PEG segments with molecular weights of from about 1.8 kDa to about 10 kDa, or from about 2 kDa to about 5 kDa, or from about 2.5 kDa to about 4.5 kDa, or from about 2.5 kDa to about 3.5 kDa. In some embodiments, at least about 10%, or at least about 25%, or at least about 50%, or at least about 75%, or at least about 90%, or at least about 95%, or at least about 99% or more of the hydrophobic regions in these coatings have molecular weights within these ranges. In some embodiments, the coatings are biologically inert. Compounds that generate both a hydrophilic surface and an uncharged or substantially neutrally-charged surface can be biologically inert.

Representative coatings according to the compositions and methods disclosed herein can include molecules having, for example, segments such as PLGA segments with molecular weights of at least about 4 kDa, or at least about 8 kDa, or at least about 12 kDa, or at least about 16 kDa, or at least about 20 kDa, or at least about 24 kDa, or at least about 28 kDa, or at least about 32 kDa, or at least about 36 kDa, or at least about 40 kDa, or at least about 44 kDa, of at least about 48 kDa, or at least about 52 kDa, or at least about 56 kDa, or at least about 60 kDa, or at least about 64 kDa, or at least about 68 kDa, or at least about 72 kDa, or at least about 76 kDa, or at least about 80 kDa, or at least about 84 kDa, or at least about 88 kDa or more. In some embodiments, at least about 10%, or at least about 25%, or at least about 50%, or at least about 75%, or at least about 90%, or at least about 95%, or at least about 99% or more of the regions in these coatings have molecular weights within these ranges. In some embodiments, the coatings are biologically inert. Compounds that generate both a hydrophilic surface and an uncharged or substantially neutrally-charged surface can be biologically inert.

In some embodiments, s coating can include, for example, one or more of the following: anionic proteins (e.g., bovine serum albumin), surfactants (e.g., cationic surfactants such as for example dimethyldioctadecyl-ammonium bromide), sugars or sugar derivatives (e.g., cyclodextrin), nucleic acids, polymers (e.g., heparin), mucolytic agents, N-acetylcysteine, mugwort, bromelain, papain, clerodendrum, acetylcysteine, bromhexine, carbocisteine, eprazinone, mesna, ambroxol, sobrerol, domiodol, letosteine, stepronin, tiopronin, gelsolin, thymosin β4, dornase alfa, neltenexine, erdosteine, various Dnases including rhDNase, agar, agarose, alginic acid, amylopectin, amylose, beta-glucan, callose, carrageenan, cellodextrins, cellulin, cellulose, chitin, chitosan, chrysolaminarin, curdlan, cyclodextrin, dextrin, ficoll, fructan, fucoidan, galactomannan, gellan gum, glucan, glucomannan, glycocalyx, glycogen, hemicellulose, hydroxyethyl starch, kefiran, laminarin, mucilage, glycosaminoglycan, natural gum, paramylon, pectin, polysaccharide peptide, schizophyllan, sialyl lewis x, starch, starch gelatinization, sugammadex, xanthan gum, xyloglucan, L-phosphatidylcholine (PC), 1,2-dipalmitoylphosphatidycholine (DPPC), oleic acid, sorbitan trioleate, sorbitan monooleate, sorbitan monolaurate, polyoxyethylene (20) sorbitan monolaurate, polyoxyethylene (20) sorbitan monooleate, natural lecithin, oleyl polyoxyethylene (2) ether, stearyl polyoxyethylene (2) ether, polyoxyethylene (4) lauryl ether, block copolymers of oxyethylene and oxypropylene, synthetic lecithin, diethylene glycol dioleate, tetrahydrofurfuryl oleate, ethyl oleate, isopropyl myristate, glyceryl monooleate, glyceryl monostearate, glyceryl monoricinoleate, cetyl alcohol, stearyl alcohol, polyethylene glycol 400, cetyl pyridinium chloride, benzalkonium chloride, olive oil, glyceryl monolaurate, corn oil, cotton seed oil, sunflower seed oil, lecithin, oleic acid, sorbitan trioleate, and combinations of two or more of any of the foregoing.

A particle-coating combinations can be made up of any combination of particle and coating substances disclosed or suggested herein. Examples of such combinations include, for example, polystyrene-PEG, or PLGA-Pluronic® F-127.

In one aspect of the present invention, an effective amount of an active compound as described herein is incorporated into a nanoparticle, e.g. for convenience of delivery and/or extended release delivery. The use of materials in nanoscale provides one the ability to modify fundamental physical properties such as solubility, diffusivity, blood circulation half-life, drug release characteristics, and/or immunogenicity. These nanoscale agents may provide more effective and/or more convenient routes of administration, lower therapeutic toxicity, extend the product life cycle, and ultimately reduce health-care costs. As therapeutic delivery systems, nanoparticles can allow targeted delivery and controlled release.

In another aspect of the present invention, the nanoparticle or microparticle is coated with a surface agent that facilitates passage of the particle through mucus. Said nanoparticles and microparticles have a higher concentration of surface agent than has been previously achieved, leading to the unexpected property of extremely fast diffusion through mucus. The present invention further comprises a method of producing said particles. The present invention further comprises methods of using said particles to treat a patient.

A number of companies have developed microparticles for treatment of eye disorders that can be used in conjunction with the present invention. For example, Allergan has disclosed a biodegradable microsphere to deliver a therapeutic agent that is formulated in a high viscosity carrier suitable for intraocular injection or to treat a non-ocular disorder (see U.S. publication 2010/0074957 and U.S. publication 2015/0147406). In one embodiment, the '957 application describes a biocompatible, intraocular drug delivery system that includes a plurality of biodegradable microspheres, a therapeutic agent, and a viscous carrier, wherein the carrier has a viscosity of at least about 10 cps at a shear rate of 0.1/second at 25° C. Allergan has also disclosed a composite drug delivery material that can be injected into the eye of a patient that includes a plurality of microparticles dispersed in a media, wherein the microparticles contain a drug and a biodegradable or bioerodible coating and the media includes the drug dispersed in a depot-forming material, wherein the media composition may gel or solidify on injection into the eye (see WO 2013/112434 A1, claiming priority to Jan. 23, 2012). Allergan states that this invention can be used to provide a depot means to implant a solid sustained drug delivery system into the eye without an incision. In general, the depot on injection transforms to a material that has a viscosity that may be difficult or impossible to administer by injection. In addition, Allergan has disclosed biodegradable microspheres between 40 and 200 μm in diameter, with a mean diameter between 60 and 150 μm that are effectively retained in the anterior chamber of the eye without producing hyperemia, see, US 2014/0294986. The microspheres contain a drug effective for an ocular condition with greater than seven day release following administration to the anterior chamber of the eye. The administration of these large particles is intended to overcome the disadvantages of injecting 1-30 μm particles which are generally poorly tolerated.

In another embodiment any of the above delivery systems can be used to facilitate or enhance delivery through mucus.

Common techniques for preparing particles include, but are not limited to, solvent evaporation, solvent removal, spray drying, phase inversion, coacervation, and low temperature casting. Suitable methods of particle formulation are briefly described below. Pharmaceutically acceptable excipients, including pH modifying agents, disintegrants, preservatives, and antioxidants, can optionally be incorporated into the particles during particle formation.

### Solvent Evaporation

In this method, the drug (or polymer matrix and one or more Drugs) is dissolved in a volatile organic solvent, such as methylene chloride. The organic solution containing the drug is then suspended in an aqueous solution that contains a surface active agent such as poly(vinyl alcohol). The resulting emulsion is stirred until most of the organic solvent evaporated, leaving solid nanoparticles. The resulting nanoparticles are washed with water and dried overnight in a lyophilizer. Nanoparticles with different sizes and morphologies can be obtained by this method.

Drugs which contain labile polymers, such as certain polyanhydrides, may degrade during the fabrication process due to the presence of water. For these polymers, the following two methods, which are performed in completely anhydrous organic solvents, can be used.

### Solvent Removal

Solvent removal can also be used to prepare particles from drugs that are hydrolytically unstable. In this method, the drug (or polymer matrix and one or more Drugs) is dispersed or dissolved in a volatile organic solvent such as methylene chloride. This mixture is then suspended by stirring in an organic oil (such as silicon oil) to form an emulsion. Solid particles form from the emulsion, which can subsequently be isolated from the supernatant. The external morphology of spheres produced with this technique is highly dependent on the identity of the drug.

In one embodiment a compound of the present invention is administered to a patient in need thereof as particles formed by solvent removal. In another embodiment the present invention provides particles formed by solvent removal comprising a compound of the present invention and one or more pharmaceutically acceptable excipients as defined herein. In another embodiment the particles formed by solvent removal comprise a compound of the present invention and an additional therapeutic agent. In a further embodiment the particles formed by solvent removal comprise a compound of the present invention, an additional therapeutic agent, and one or more pharmaceutically acceptable excipients. In another embodiment any of the described particles formed by solvent removal can be formulated into a tablet and then coated to form a coated tablet. In an alternative embodiment the particles formed by solvent removal are formulated into a tablet but the tablet is uncoated.

### Spray Drying

In this method, the drug (or polymer matrix and one or more Drugs) is dissolved in an organic solvent such as methylene chloride. The solution is pumped through a micronizing nozzle driven by a flow of compressed gas, and the resulting aerosol is suspended in a heated cyclone of air, allowing the solvent to evaporate from the micro droplets, forming particles. Particles ranging between 0.1-10 microns can be obtained using this method.

In one embodiment a compound of the present invention is administered to a patient in need thereof as a spray dried dispersion (SDD). In another embodiment the present invention provides a spray dried dispersion (SDD) comprising a compound of the present invention and one or more pharmaceutically acceptable excipients as defined herein. In another embodiment the SDD comprises a compound of the present invention and an additional therapeutic agent. In a further embodiment the SDD comprises a compound of the present invention, an additional therapeutic agent, and one or more pharmaceutically acceptable excipients. In another embodiment any of the described spray dried dispersions can be coated to form a coated tablet. In an alternative embodiment the spray dried dispersion is formulated into a tablet but is uncoated.

### Phase Inversion

Particles can be formed from drugs using a phase inversion method. In this method, the drug (or polymer matrix and one or more Drugs) is dissolved in a “good” solvent, and the solution is poured into a strong non solvent for the drug to spontaneously produce, under favorable conditions, microparticles or nanoparticles. The method can be used to produce nanoparticles in a wide range of sizes, including, for example, about 100 nanometers to about 10 microns, typically possessing a narrow particle size distribution.

In one embodiment a compound of the present invention is administered to a patient in need thereof as particles formed by phase inversion. In another embodiment the present invention provides particles formed by phase inversion comprising a compound of the present invention and one or more pharmaceutically acceptable excipients as defined herein. In another embodiment the particles formed by phase inversion comprise a compound of the present invention and an additional therapeutic agent. In a further embodiment the particles formed by phase inversion comprise a compound of the present invention, an additional therapeutic agent, and one or more pharmaceutically acceptable excipients. In another embodiment any of the described particles formed by phase inversion can be formulated into a tablet and then coated to form a coated tablet. In an alternative embodiment the particles formed by phase inversion are formulated into a tablet but the tablet is uncoated.

### Coacervation

Techniques for particle formation using coacervation are known in the art, for example, in GB-B-929 406; GB-B-929 40 1; and U.S. Pat. Nos. 3,266,987, 4,794,000, and 4,460,563. Coacervation involves the separation of a drug (or polymer matrix and one or more Drugs) solution into two immiscible liquid phases. One phase is a dense coacervate phase, which contains a high concentration of the drug, while the second phase contains a low concentration of the drug. Within the dense coacervate phase, the drug forms nanoscale or microscale droplets, which harden into particles. Coacervation may be induced by a temperature change, addition of a non-solvent or addition of a micro-salt (simple coacervation), or by the addition of another polymer thereby forming an interpolymer complex (complex coacervation).

In one embodiment a compound of the present invention is administered to a patient in need thereof as particles formed by coacervation. In another embodiment the present invention provides particles formed by coacervation comprising a compound of the present invention and one or more pharmaceutically acceptable excipients as defined herein. In another embodiment the particles formed by coacervation comprise a compound of the present invention and an additional therapeutic agent. In a further embodiment the particles formed by coacervation comprise a compound of the present invention, an additional therapeutic agent, and one or more pharmaceutically acceptable excipients. In another embodiment any of the described particles formed by coacervation can be formulated into a tablet and then coated to form a coated tablet. In an alternative embodiment the particles formed by coacervation are formulated into a tablet but the tablet is uncoated.

### Low Temperature Casting

Methods for very low temperature casting of controlled release microspheres are described in U.S. Pat. No. 5,019,400 to Gombotz et al. In this method, the drug (or polymer matrix and Sunitinib) is dissolved in a solvent. The mixture is then atomized into a vessel containing a liquid non-solvent at a temperature below the freezing point of the drug solution which freezes the drug droplets. As the droplets and non-solvent for the drug are warmed, the solvent in the droplets thaws and is extracted into the non-solvent, hardening the microspheres.

In one embodiment a compound of the present invention is administered to a patient in need thereof as particles formed by low temperature casting. In another embodiment the present invention provides particles formed by low temperature casting comprising a compound of the present invention and one or more pharmaceutically acceptable excipients as defined herein. In another embodiment the particles formed by low temperature casting comprise a compound of the present invention and an additional therapeutic agent. In a further embodiment the particles formed by low temperature casting comprise a compound of the present invention, an additional therapeutic agent, and one or more pharmaceutically acceptable excipients. In another embodiment any of the described particles formed by low temperature casting can be formulated into a tablet and then coated to form a coated tablet. In an alternative embodiment the particles formed by low temperature casting are formulated into a tablet but the tablet is uncoated.

III. Controlled Release of Therapeutic Agent

The rate of release of the therapeutic agent can be related to the concentration of therapeutic agent dissolved in polymeric material. In many embodiments, the polymeric composition includes non-therapeutic agents that are selected to provide a desired solubility of the therapeutic agent. The selection of polymer can be made to provide the desired solubility of the therapeutic agent in the matrix, for example, a hydrogel may promote solubility of hydrophilic material. In some embodiments, functional groups can be added to the polymer to increase the desired solubility of the therapeutic agent in the matrix. In some embodiments, additives may be used to control the release kinetics of therapeutic agent, for example, the additives may be used to control the concentration of therapeutic agent by increasing or decreasing solubility of the therapeutic agent in the polymer so as to control the release kinetics of the therapeutic agent. The solubility may be controlled by including appropriate molecules and/or substances that increase and/or decrease the solubility of the dissolved from of the therapeutic agent to the matrix. The solubility of the therapeutic agent may be related to the hydrophobic and/or hydrophilic properties of the matrix and therapeutic agent. Oils and hydrophobic molecules and can be added to the polymer to increase the solubility of hydrophobic treatment agent in the matrix.

Instead of or in addition to controlling the rate of migration based on the concentration of therapeutic agent dissolved in the matrix, the surface area of the polymeric composition can be controlled to attain the desired rate of drug migration out of the composition. For example, a larger exposed surface area will increase the rate of migration of the active agent to the surface, and a smaller exposed surface area will decrease the rate of migration of the active agent to the surface. The exposed surface area can be increased in any number of ways, for example, by any of castellation of the exposed surface, a porous surface having exposed channels connected with the tear or tear film, indentation of the exposed surface, protrusion of the exposed surface. The exposed surface can be made porous by the addition of salts that dissolve and leave a porous cavity once the salt dissolves. In the present invention, these trends can be used to decrease the release rate of the active material from the polymeric composition by avoiding these paths to quicker release. For example, the surface area can be minimized, or channels avoided.

Further, an implant may be used that includes the ability to release two or more drugs in combination, for example, the structure disclosed in U.S. Pat. No. 4,281,654 (Shell), for example, in the case of glaucoma treatment, it may be desirable to treat a patient with multiple prostaglandins or a prostaglandin and a cholinergic agent or an adrenergic antagonist (beta blocker), for example, Alphagan (Allegan, Irvine, Calif., USA), or a prostaglandin and a carbonic anhydrase inhibitor.

In addition, drug impregnated meshes may be used, for example, those disclosed in U.S. Patent Application Publication No. 2002/0055701 or layering of biostable polymers as described in U.S. Patent Application Publication No. 2005/0129731. Certain polymer processes may be used to incorporate drug into the devices, as described herein, for example, so-called “self-delivering drugs” or Polymer Drugs (Polymerix Corporation, Piscataway, N.J., USA) are designed to degrade only into therapeutically useful compounds and physiologically inert linker molecules, further detailed in U.S. Patent Application Publication No. 2005/0048121 (East), hereby incorporated by reference in its entirety. Such delivery polymers may be employed in the devices, as described herein, to provide a release rate that is equal to the rate of polymer erosion and degradation and is constant throughout the course of therapy. Such delivery polymers may be used as device coatings or in the form of microspheres for a drug depot injectable (for example, a reservoir described herein). A further polymer delivery technology may also be adapted to the devices, as described herein, for example, that described in U.S. Patent Application Publication No. 2004/0170685 (Carpenter), and technologies available from Medivas (San Diego, Calif., USA).

### EXAMPLES

### General Methods

All nonaqueous reactions were performed under an atmosphere of dry argon or nitrogen gas using anhydrous solvents. The progress of reactions and the purity of target compounds were determined using one of the two liquid chromatography (LC) methods listed below. The structure of starting materials, intermediates, and final products was confirmed by standard analytical techniques, including NMR spectroscopy and mass spectrometry.

The compounds described herein can be prepared by methods known by those skilled in the art. In one non-limiting example the disclosed compounds can be made by the schemes below.

### Example 1. Non-Limiting Examples of Compounds of Formula I, Formula II, Formula III, Formula IV, or Formula IV′

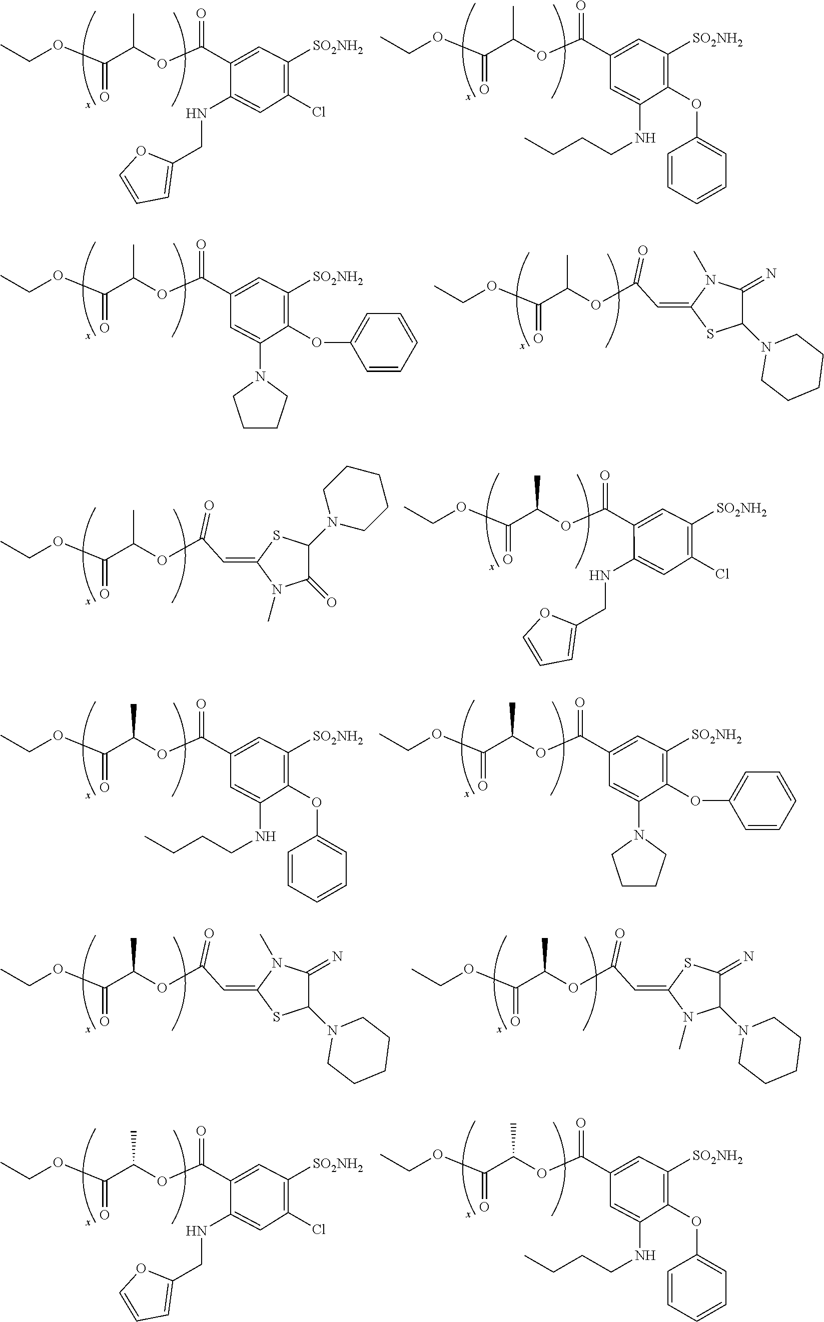

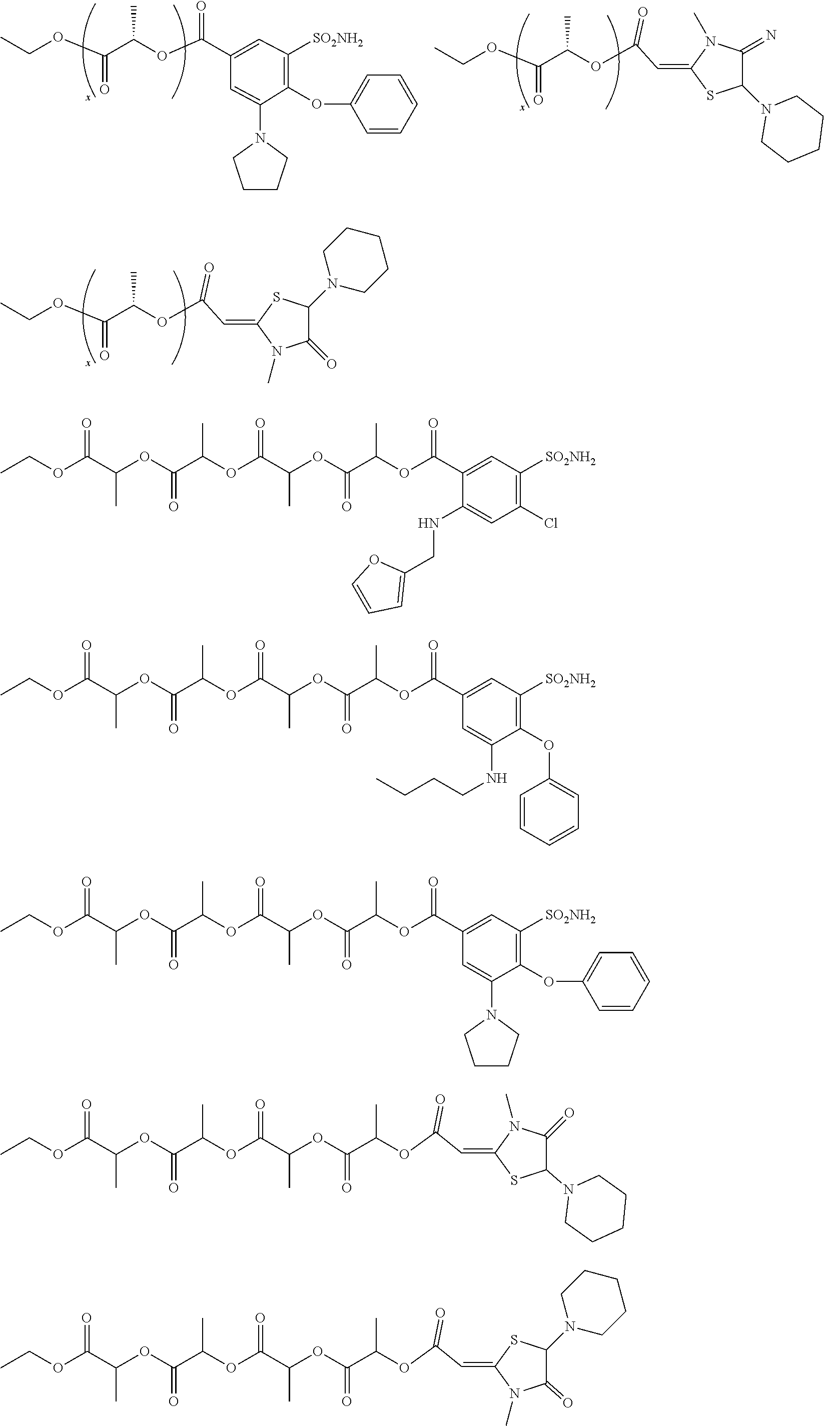

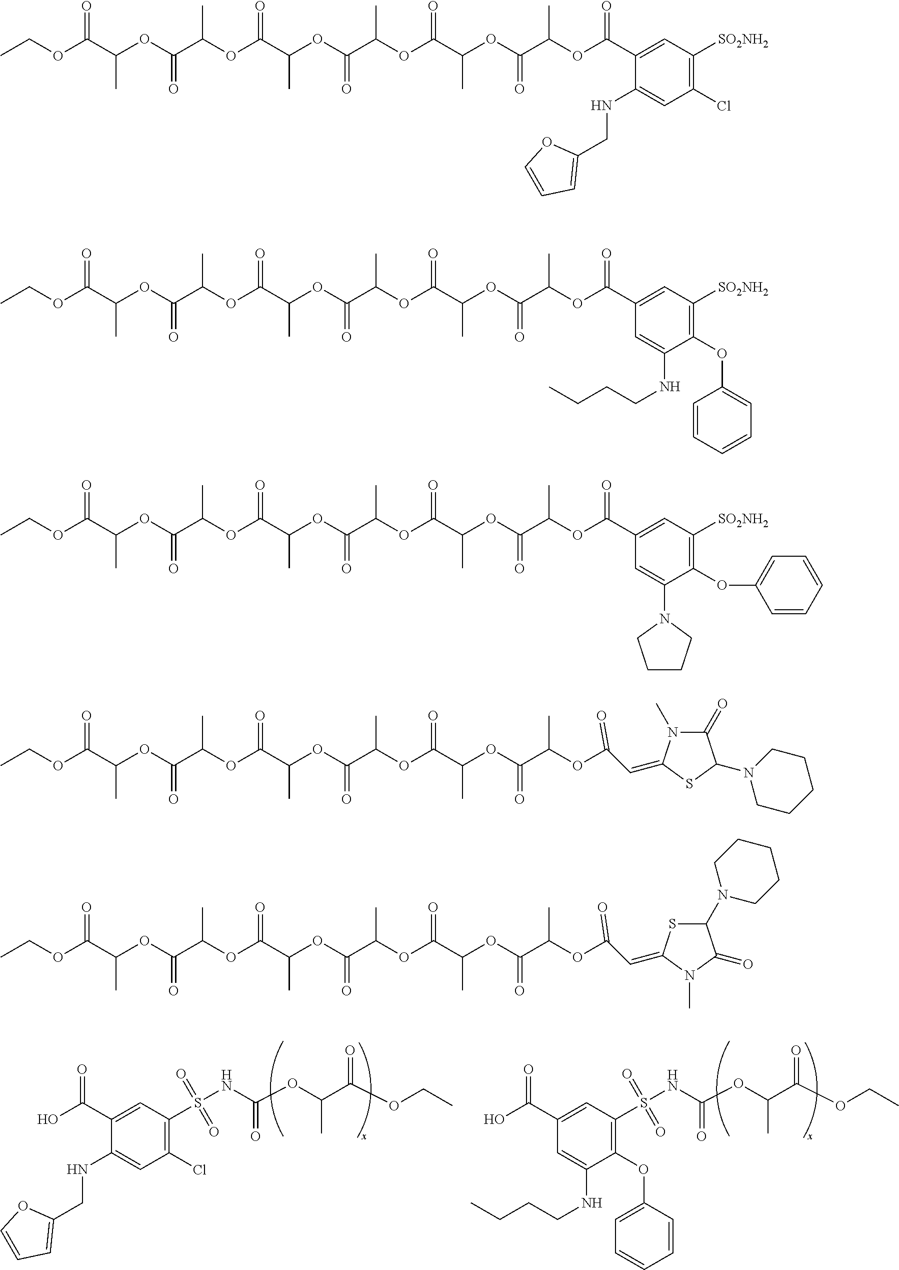

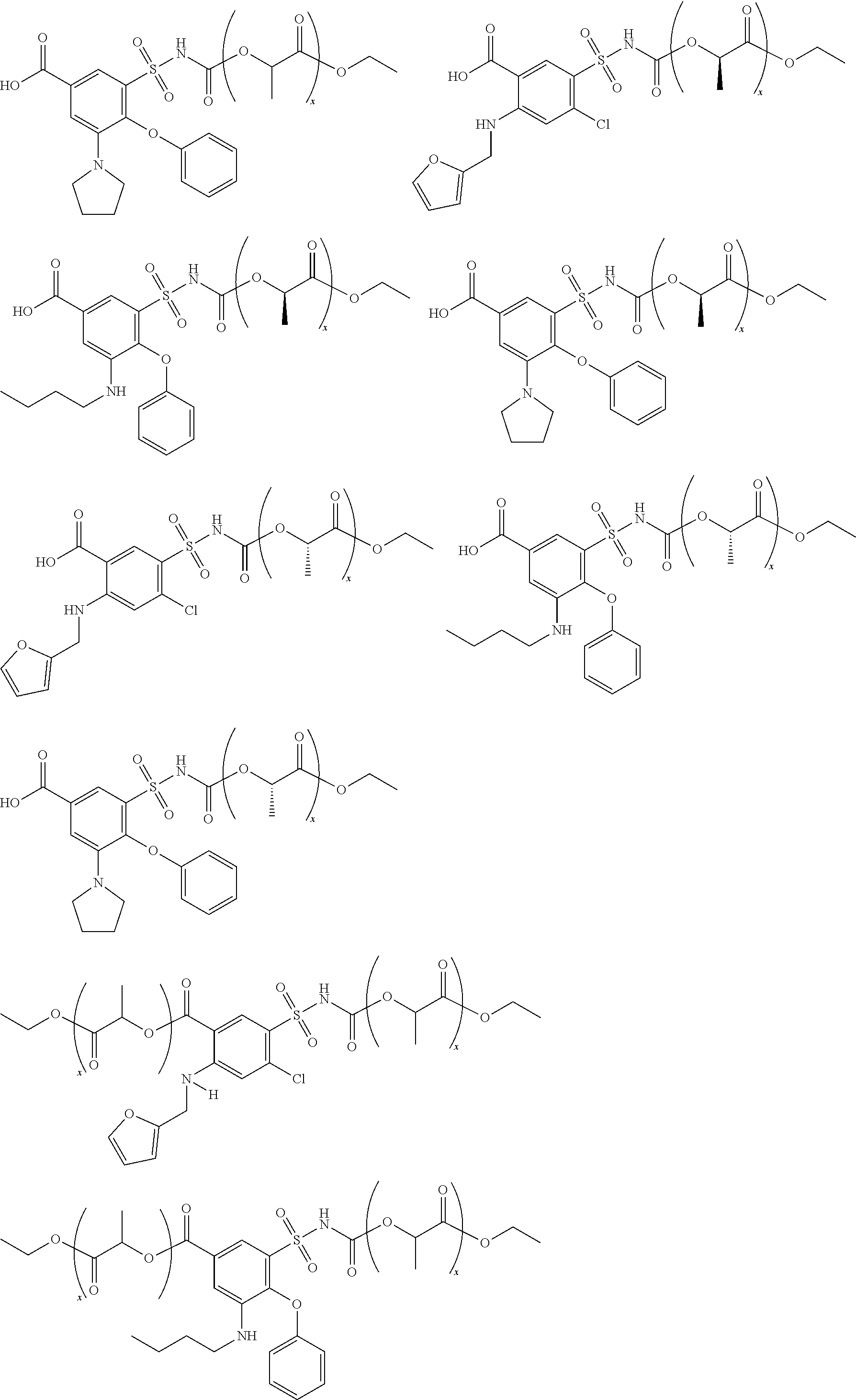

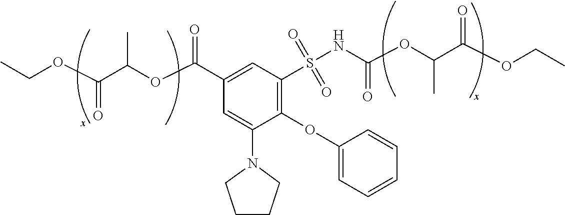

In one embodiment, x is independently an integer between 1 and 12 (1, 2, 3, 4, 5, 6, 7, 8, 9, 10, 11, or 12).

In one embodiment, x is independently an integer between 1 and 10 (1, 2, 3, 4, 5, 6, 7, 8, 9, or 10).

In one embodiment, x is independently an integer between 1 and 8 (1, 2, 3, 4, 5, 6, 7, or 8).

In one embodiment, x is independently an integer between 1 and 6 (1, 2, 3, 4, 5, or 6).

In one embodiment, x is independently an integer between 4 and 10 (4, 5, 6, 7, 8, 9, or 10).

In one embodiment, x is 4.

In one embodiment, x is 6.

In one embodiment, x is 8.

In one embodiment, x is 10.

### Example 2. Additional Non-Limiting Examples of Compounds of Formula I, Formula II, Formula III, Formula IV, or Formula IV′

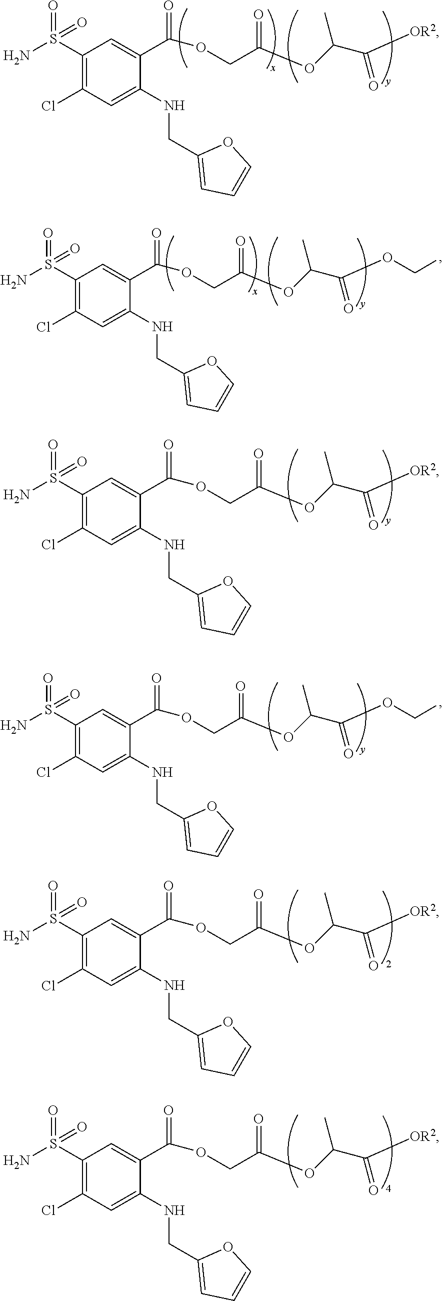

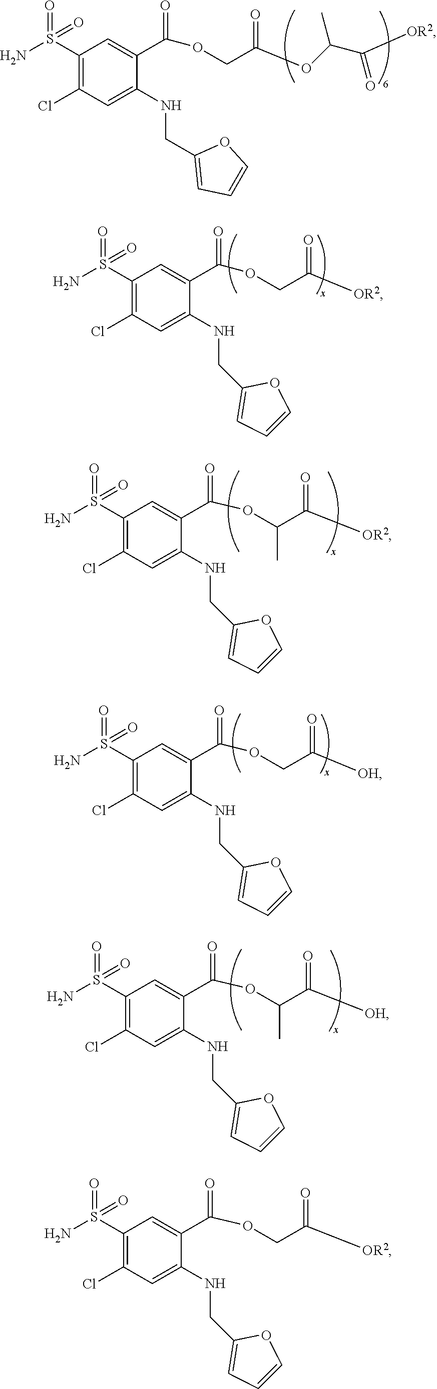

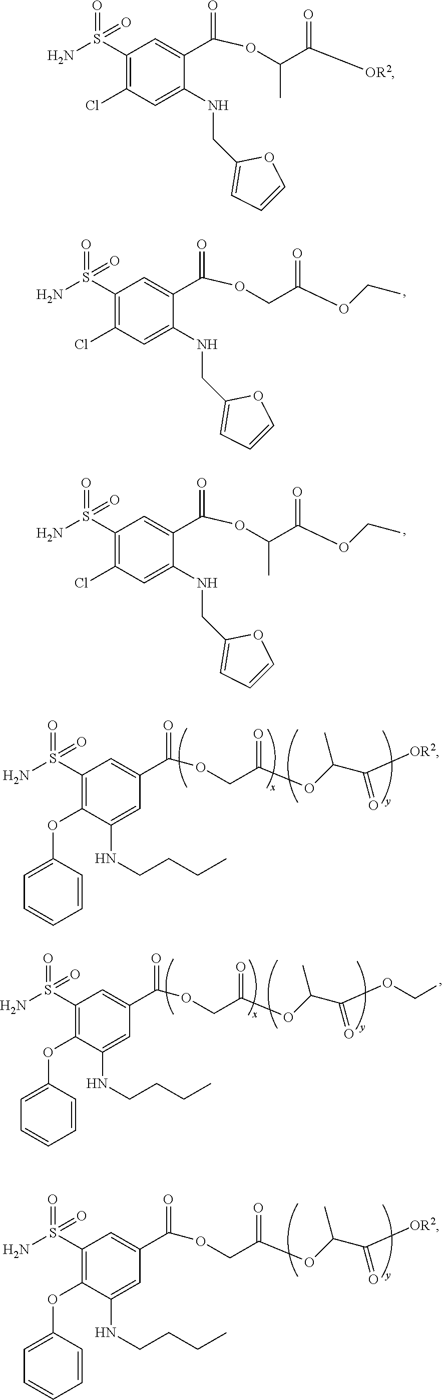

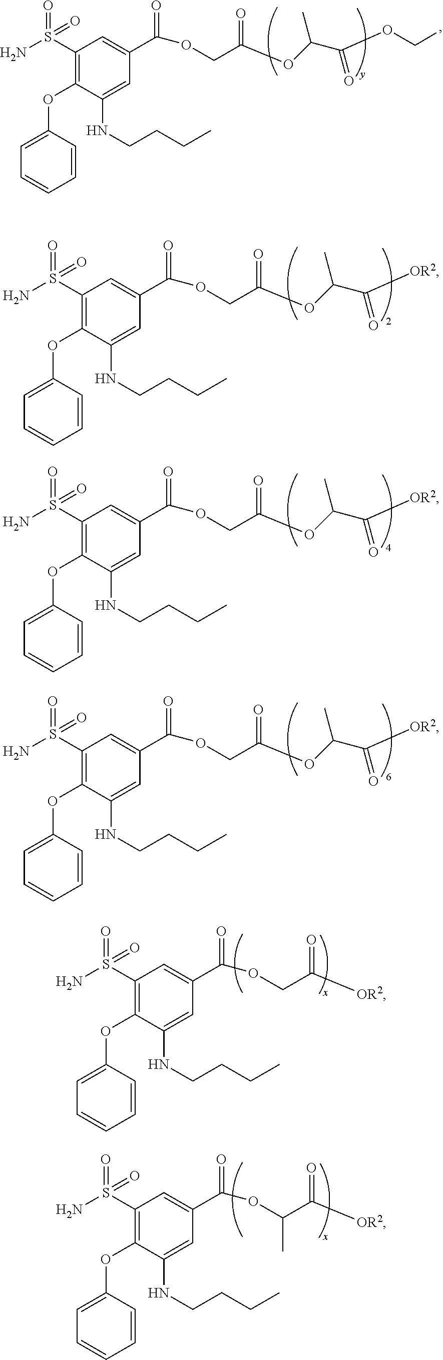

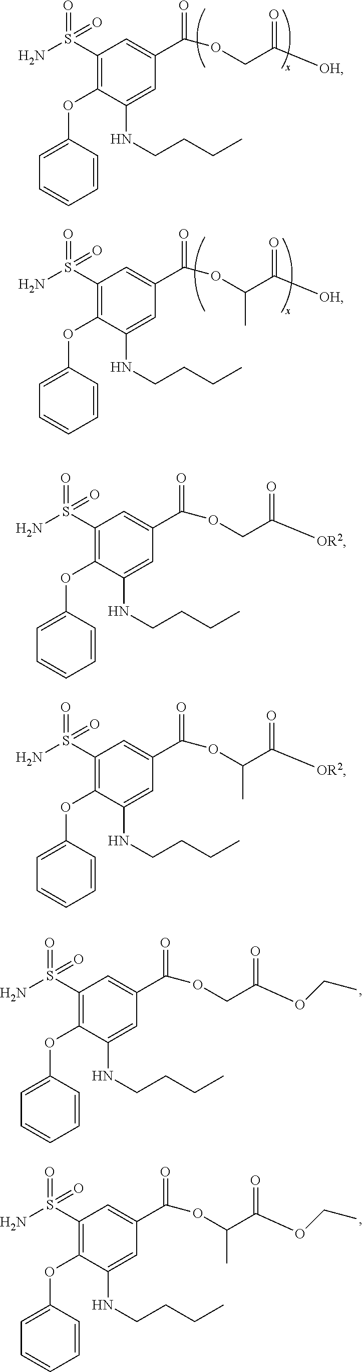

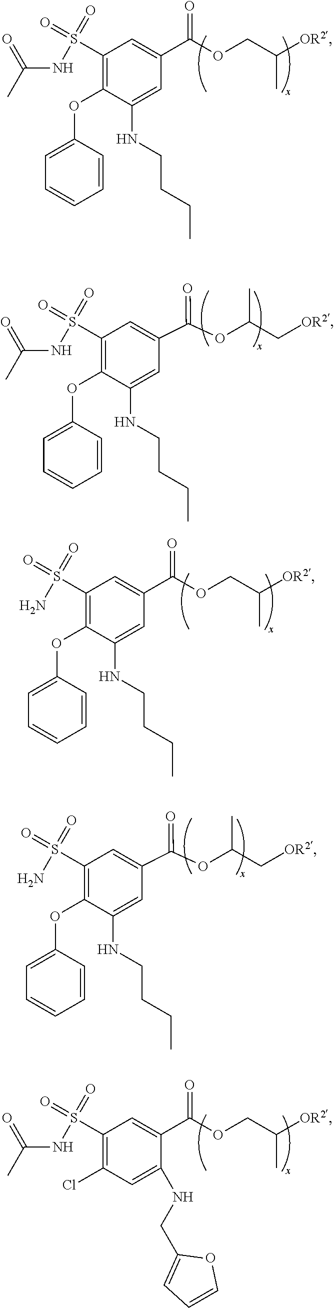

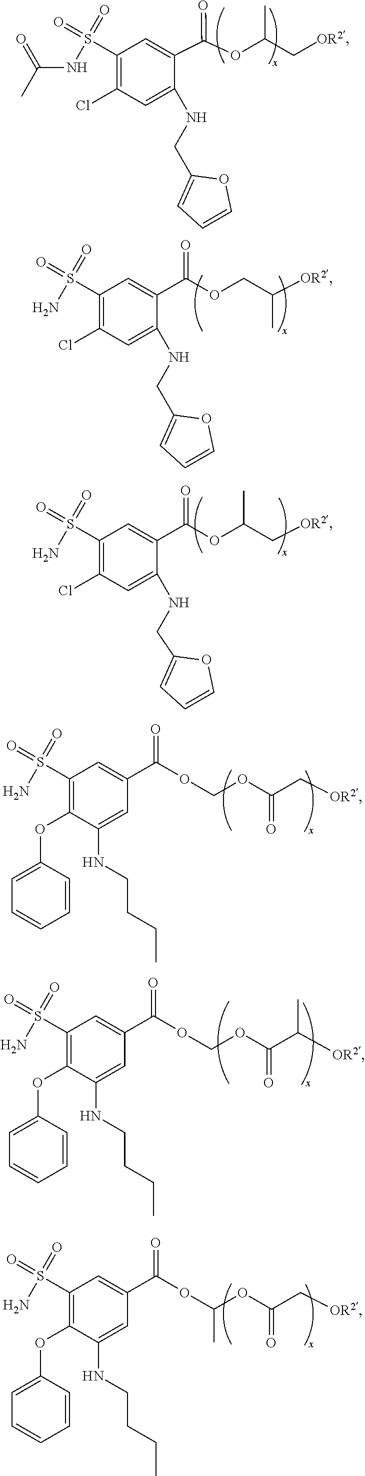

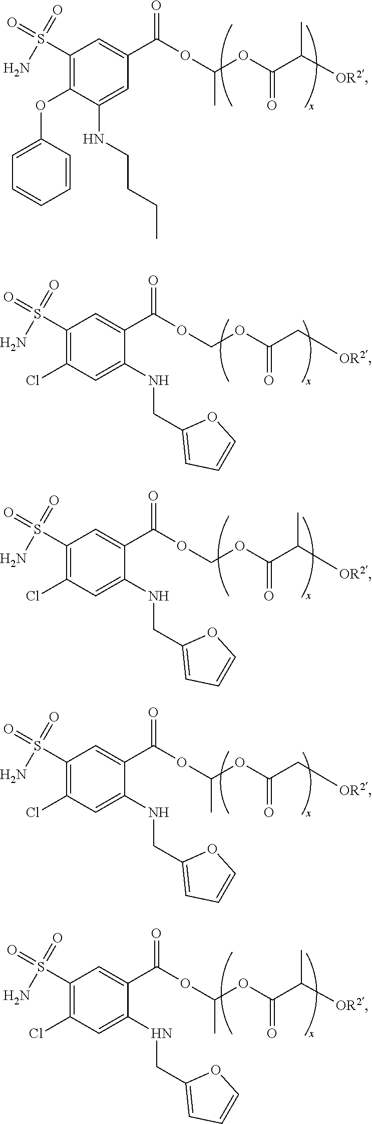

### Example 3. Non-Limiting Examples of Compounds of Formula V, Formula VI, Formula VII, Formula VIII, or Formula VIII′

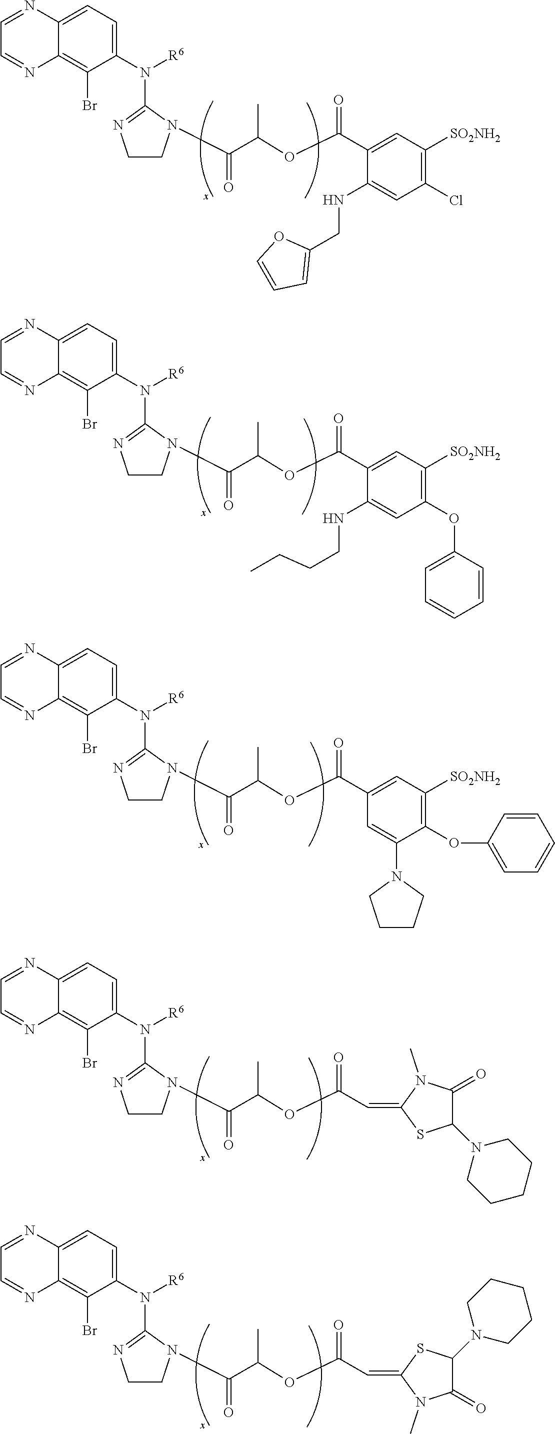

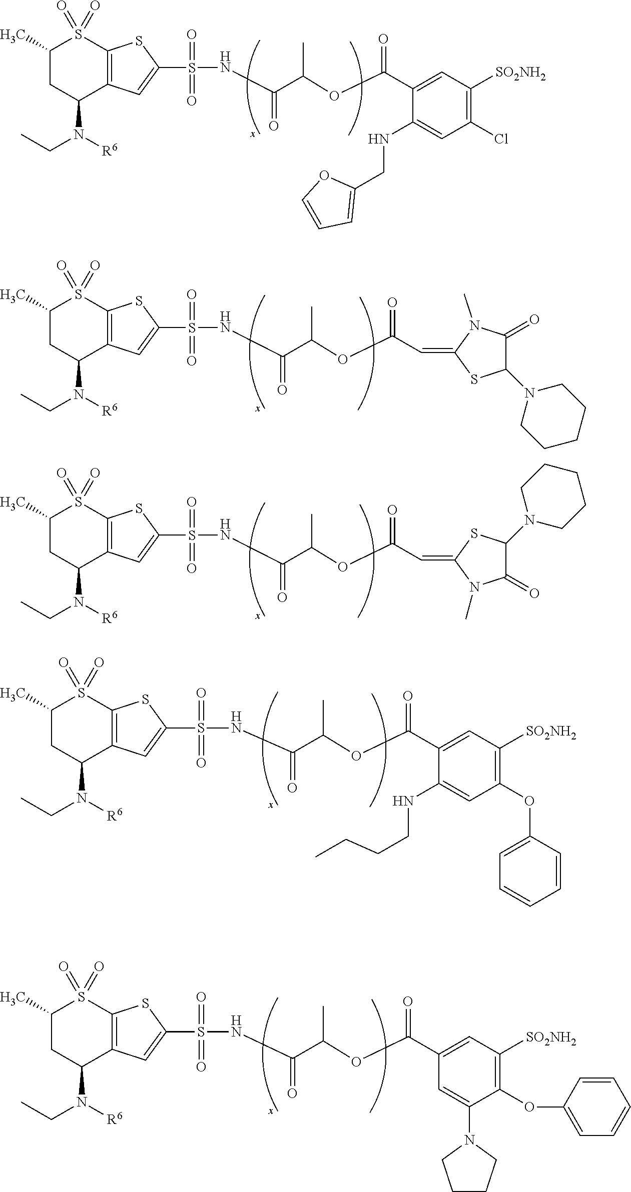

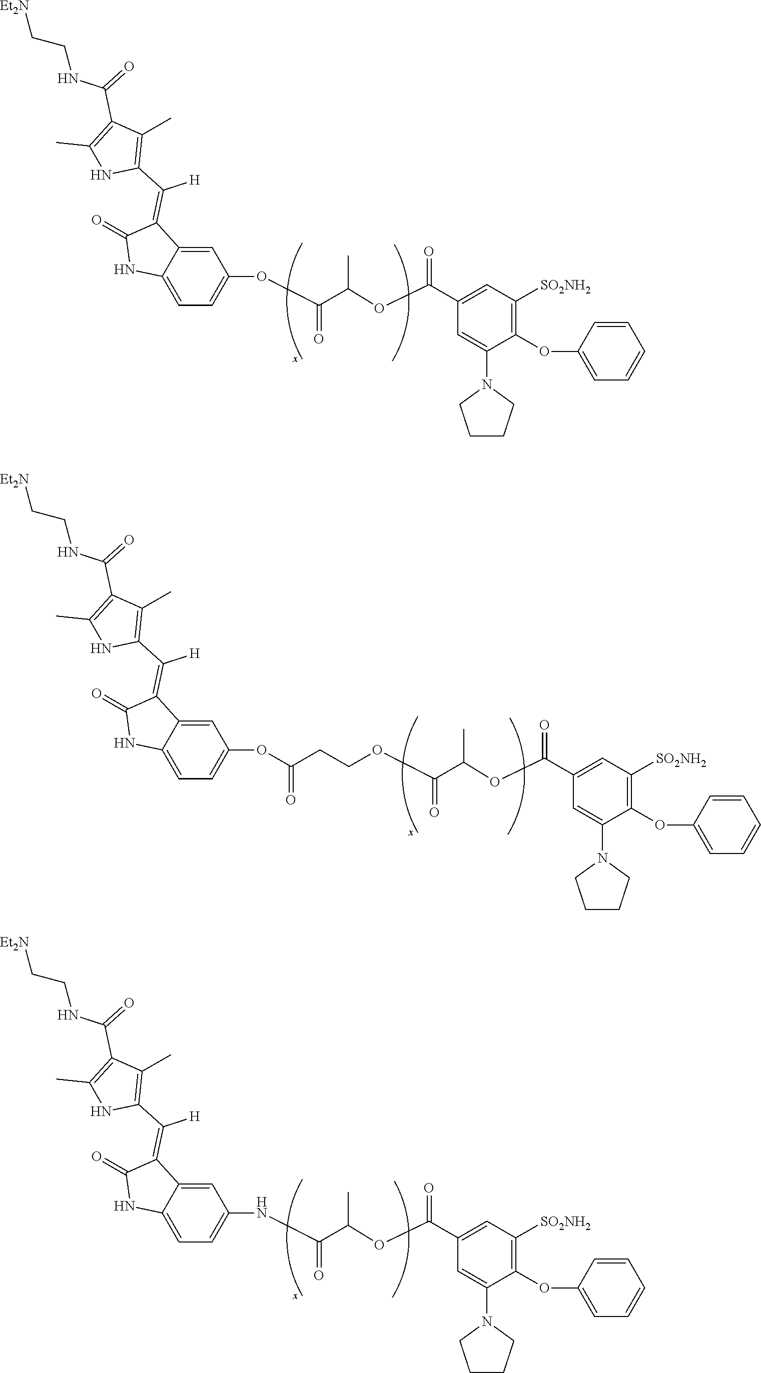

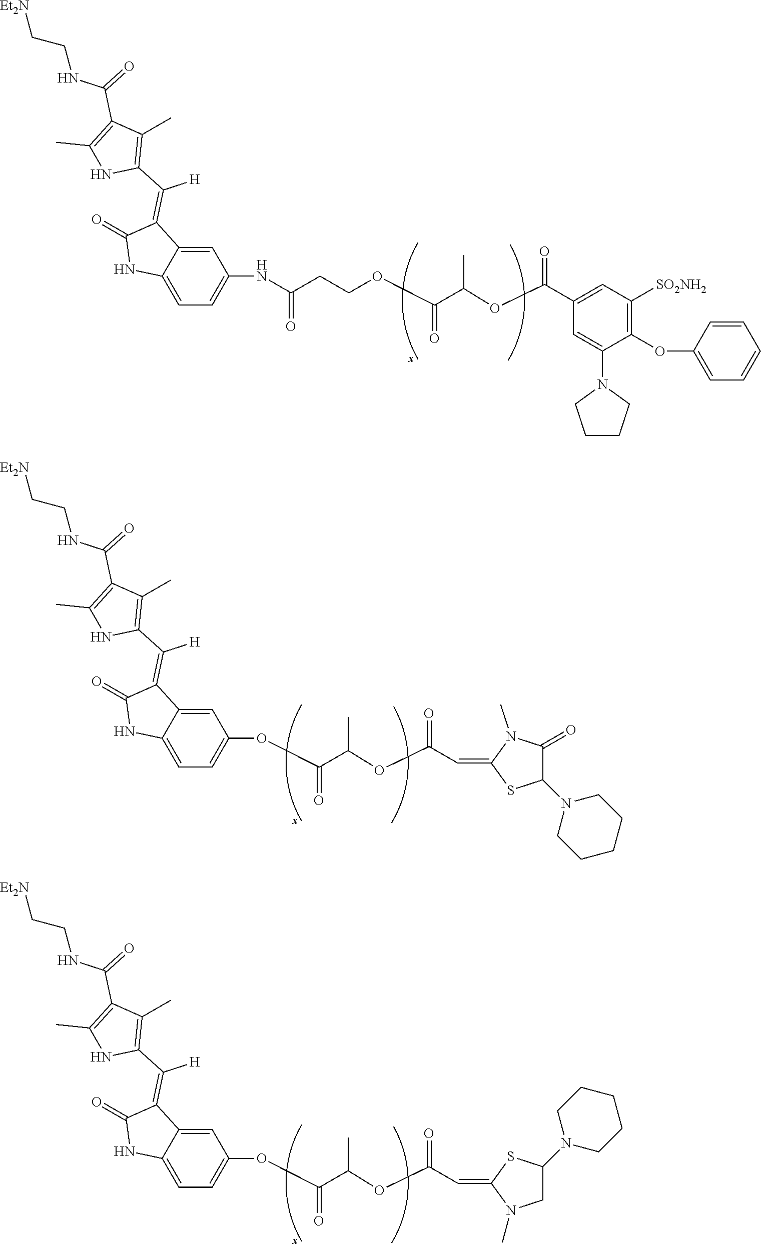

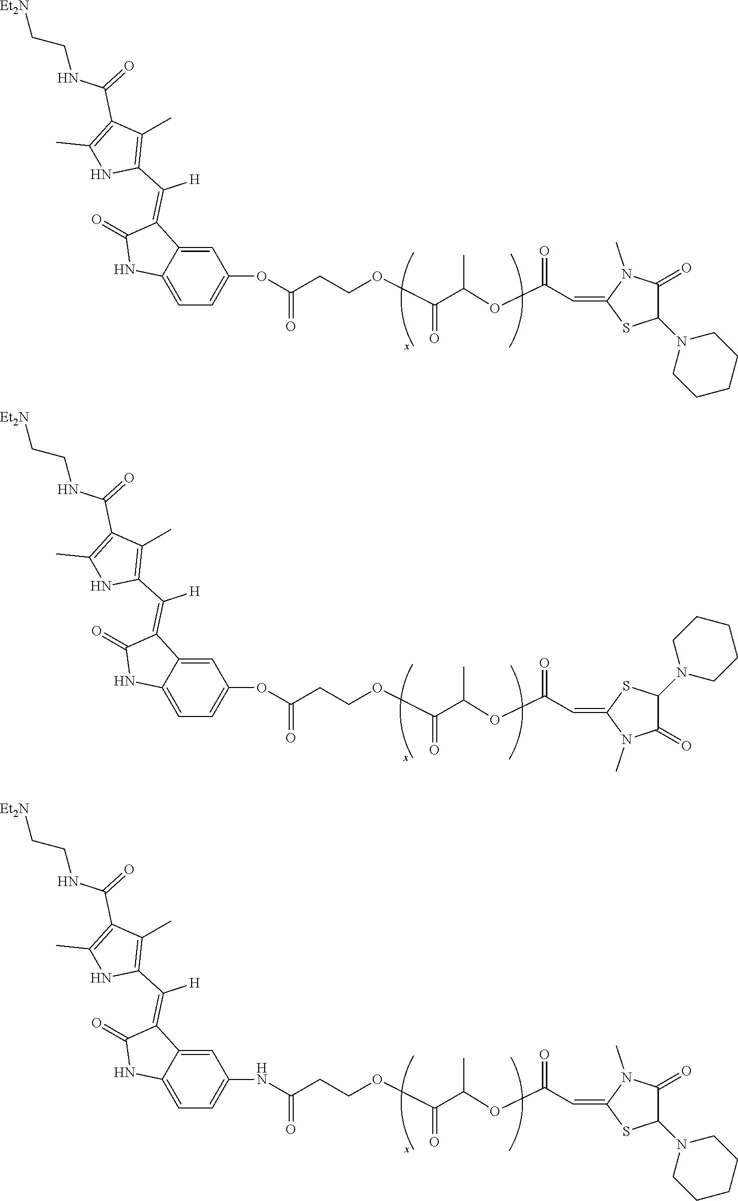

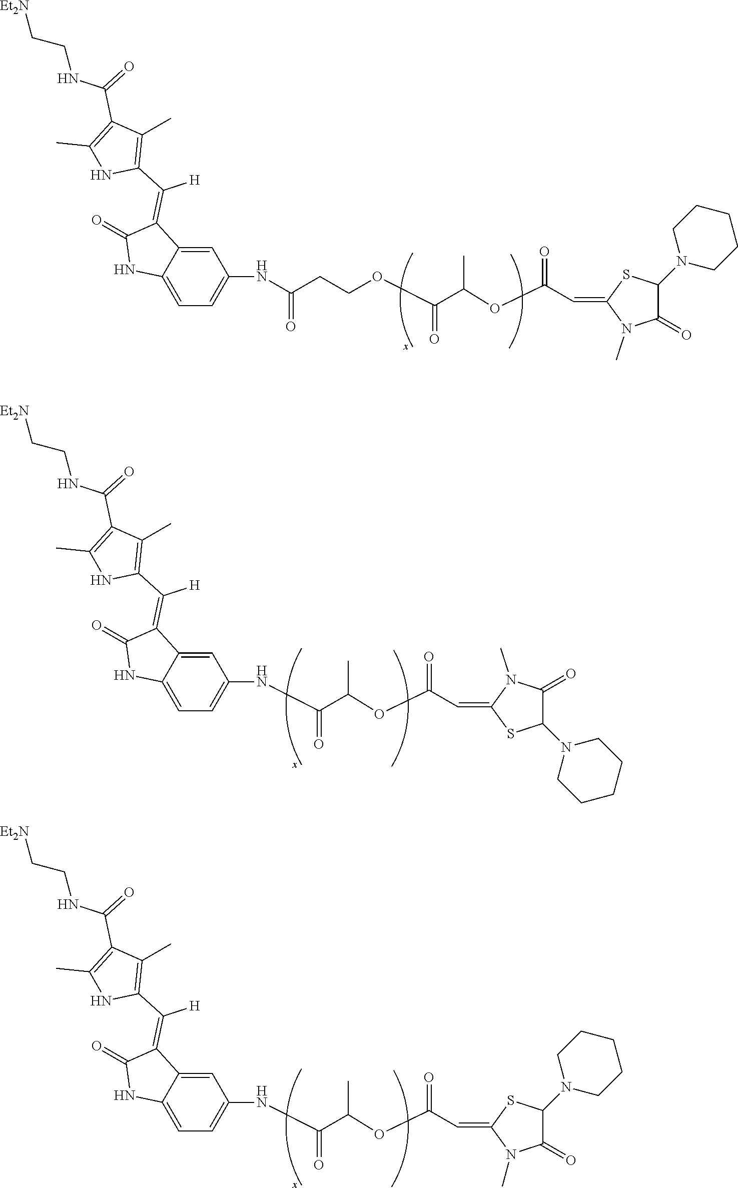

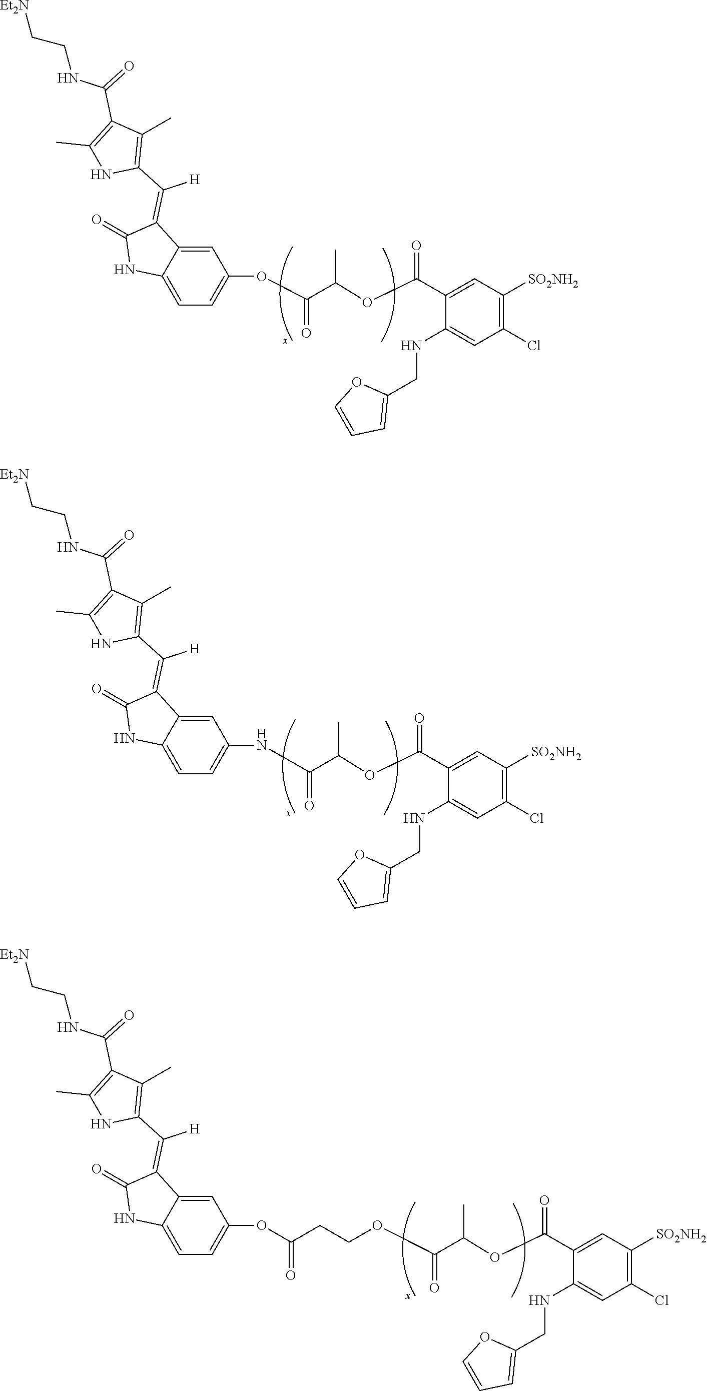

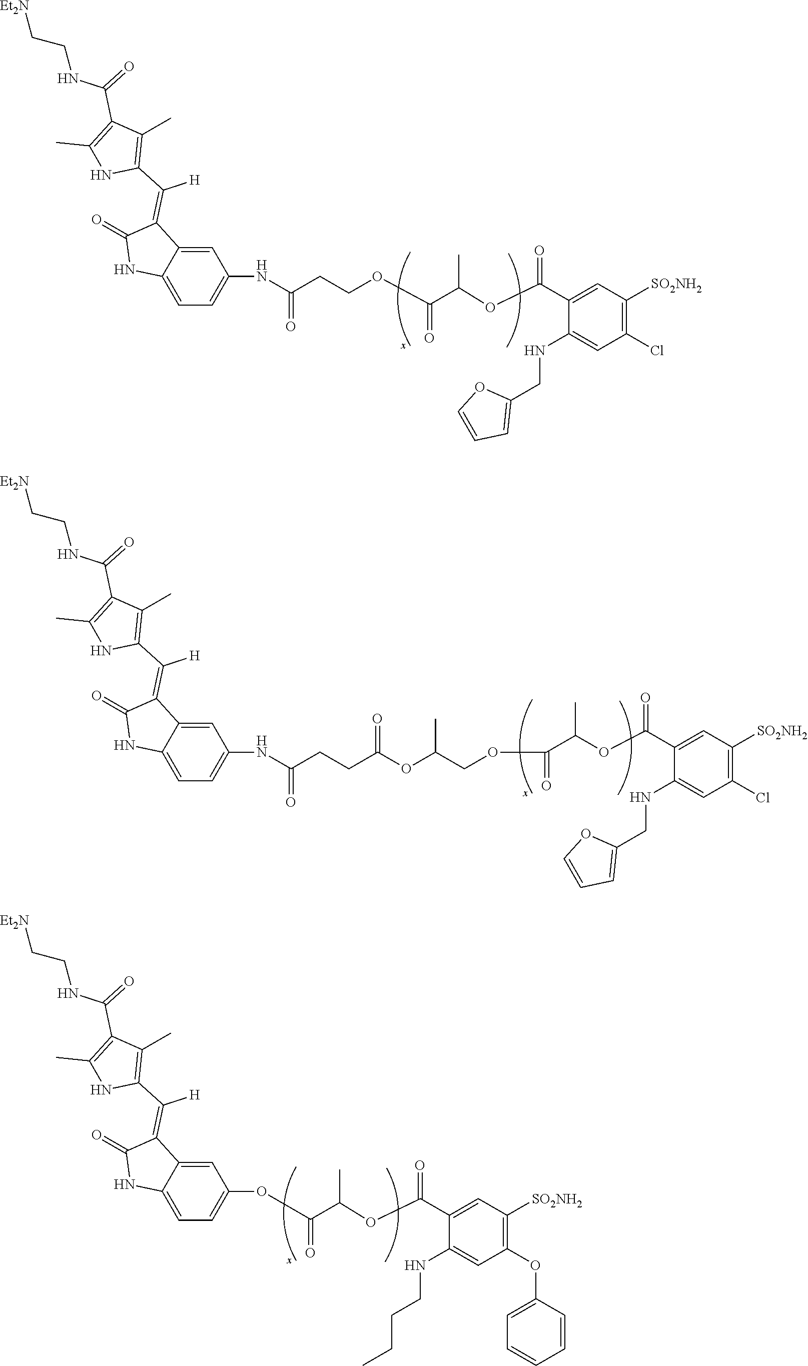

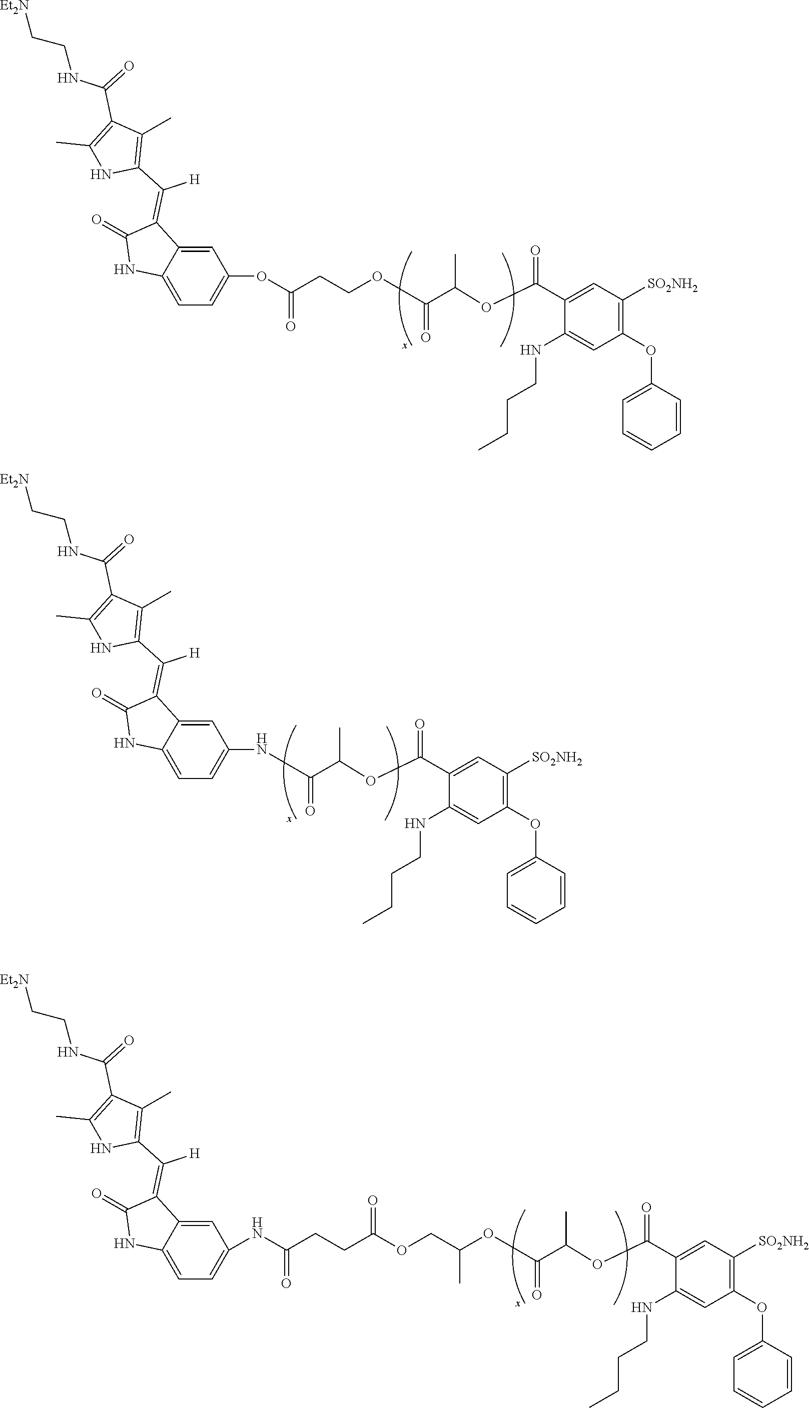

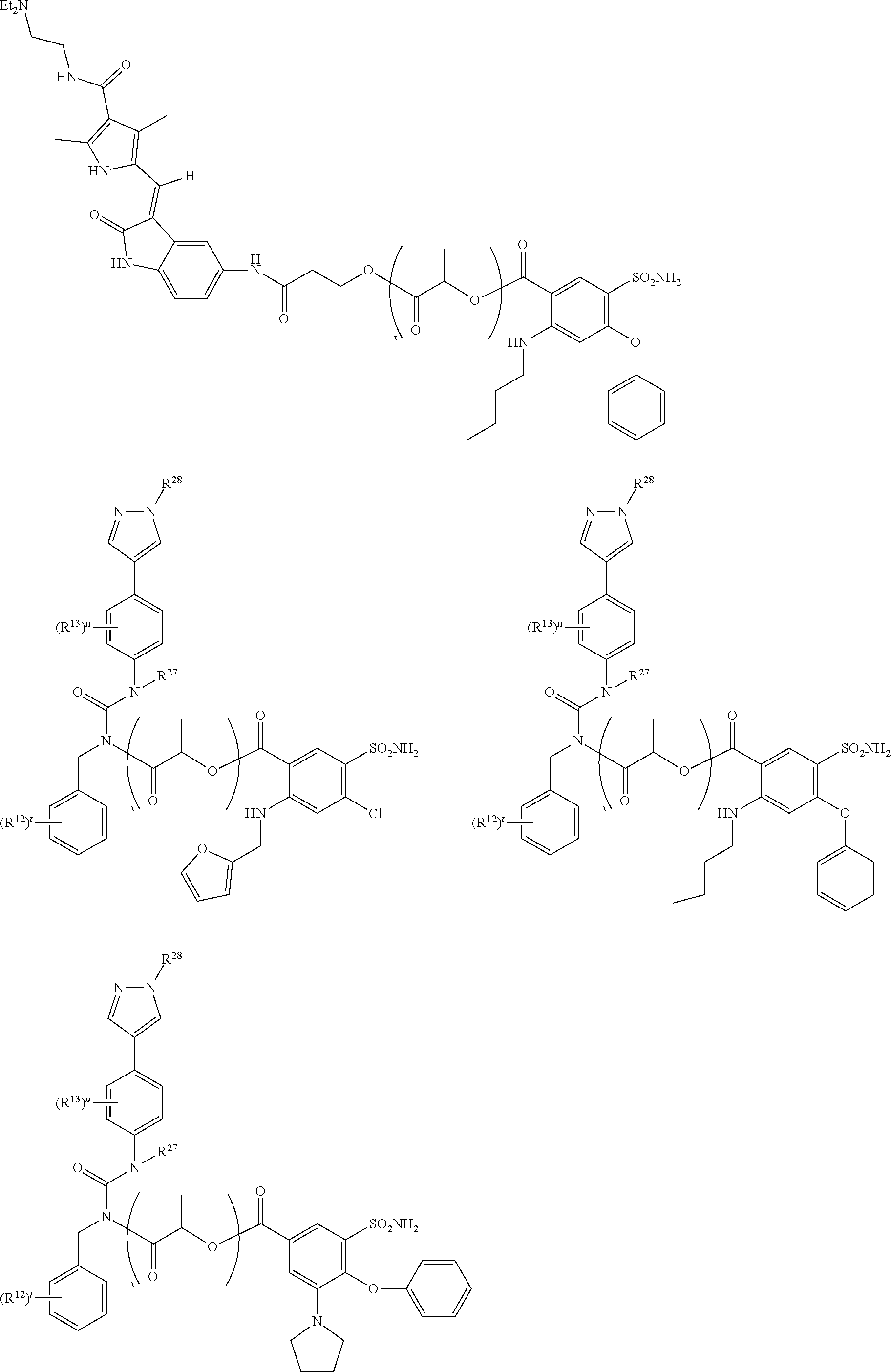

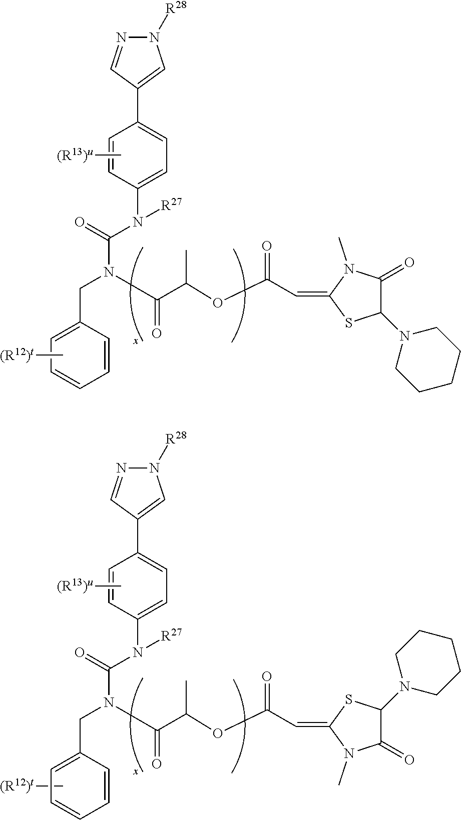

### Embodiments of x and y

In one embodiment x is 1 and y is 1.

In one embodiment x is 1 and y is 2.

In one embodiment x is 1 and y is 3.

In one embodiment x is 1 and y is 4.

In one embodiment x is 1 and y is 5.

In one embodiment x is 1 and y is 6.

In one embodiment x is 1 and y is 7.

In one embodiment x is 1 and y is 8.

In one embodiment x is 2 and y is 1.

In one embodiment x is 2 and y is 2.

In one embodiment x is 2 and y is 3.

In one embodiment x is 2 and y is 4.

In one embodiment x is 2 and y is 5.

In one embodiment x is 2 and y is 6.

In one embodiment x is 2 and y is 7.

In one embodiment x is 2 and y is 8.

In one embodiment x is 3 and y is 1.

In one embodiment x is 3 and y is 2.

In one embodiment x is 3 and y is 3.

In one embodiment x is 3 and y is 4.

In one embodiment x is 3 and y is 5.

In one embodiment x is 3 and y is 6.

In one embodiment x is 3 and y is 7.

In one embodiment x is 3 and y is 8.

In one embodiment x is 4 and y is 1.

In one embodiment x is 4 and y is 2.

In one embodiment x is 4 and y is 3.

In one embodiment x is 4 and y is 4.

In one embodiment x is 4 and y is 5.

In one embodiment x is 4 and y is 6.

In one embodiment x is 4 and y is 7.

In one embodiment x is 4 and y is 8.

In one embodiment x is 5 and y is 1.

In one embodiment x is 5 and y is 2.

In one embodiment x is 5 and y is 3.

In one embodiment x is 5 and y is 4.

In one embodiment x is 5 and y is 5.

In one embodiment x is 5 and y is 6.

In one embodiment x is 5 and y is 7.

In one embodiment x is 5 and y is 8.

In one embodiment x is 6 and y is 1.

In one embodiment x is 6 and y is 2.

In one embodiment x is 6 and y is 3.

In one embodiment x is 6 and y is 4.

In one embodiment x is 6 and y is 5.

In one embodiment x is 6 and y is 6.

In one embodiment x is 6 and y is 7.

In one embodiment x is 6 and y is 8.

In one embodiment x is 7 and y is 1.

In one embodiment x is 7 and y is 2.

In one embodiment x is 7 and y is 3.

In one embodiment x is 7 and y is 4.

In one embodiment x is 7 and y is 5.

In one embodiment x is 7 and y is 6.

In one embodiment x is 7 and y is 7.

In one embodiment x is 7 and y is 8.

In one embodiment x is 8 and y is 1.

In one embodiment x is 8 and y is 2.

In one embodiment x is 8 and y is 3.

In one embodiment x is 8 and y is 4.

In one embodiment x is 8 and y is 5.

In one embodiment x is 8 and y is 6.

In one embodiment x is 8 and y is 7.

In one embodiment x is 8 and y is 8.

### Example 4. Additional Non-Limiting Examples of Compounds of Formula V, Formula VI, Formula VII, Formula VIII, or Formula VIII′

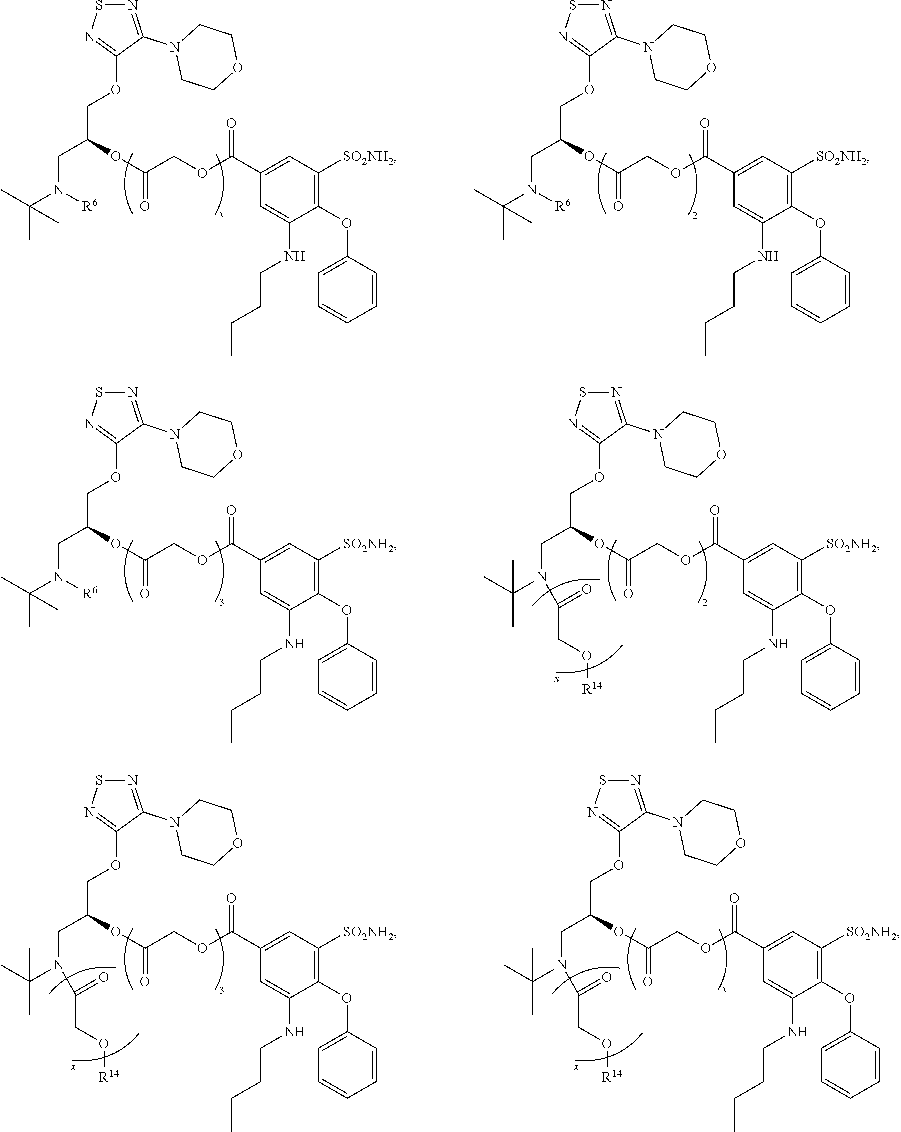

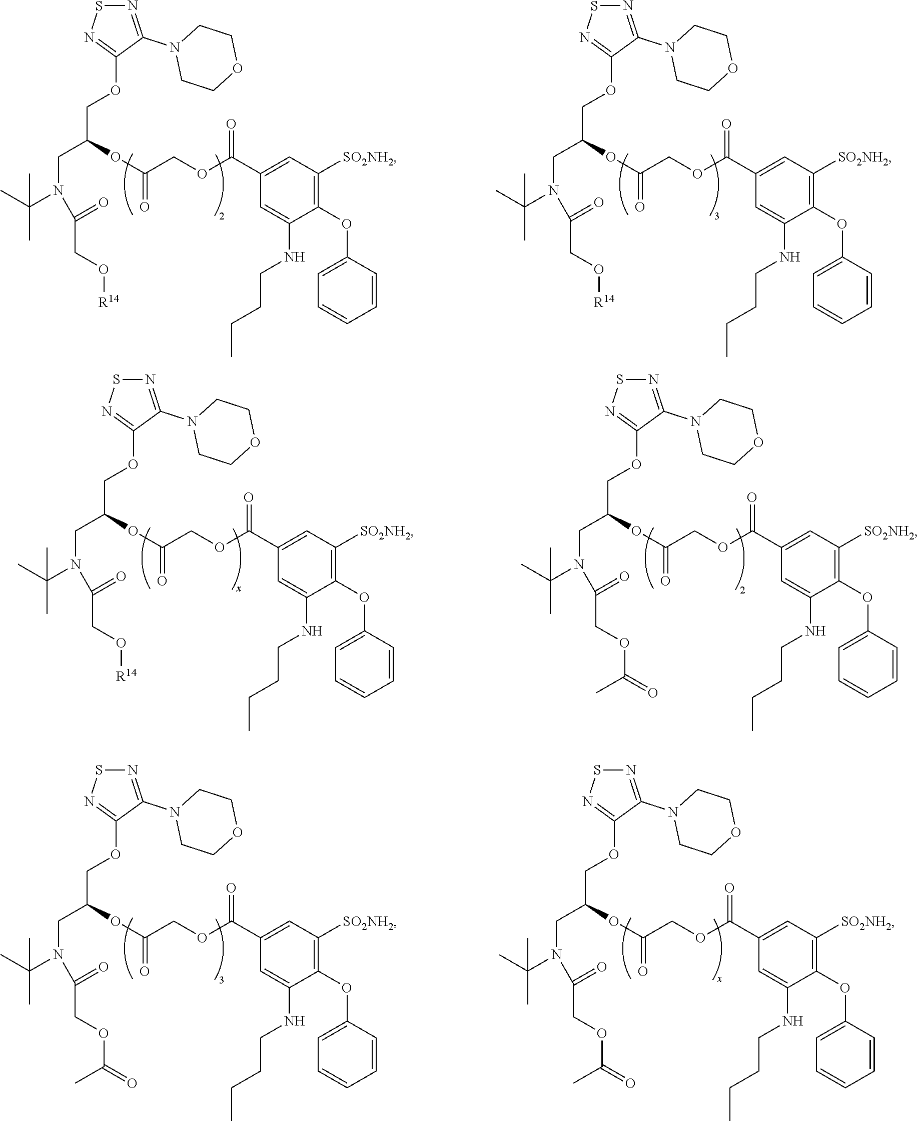

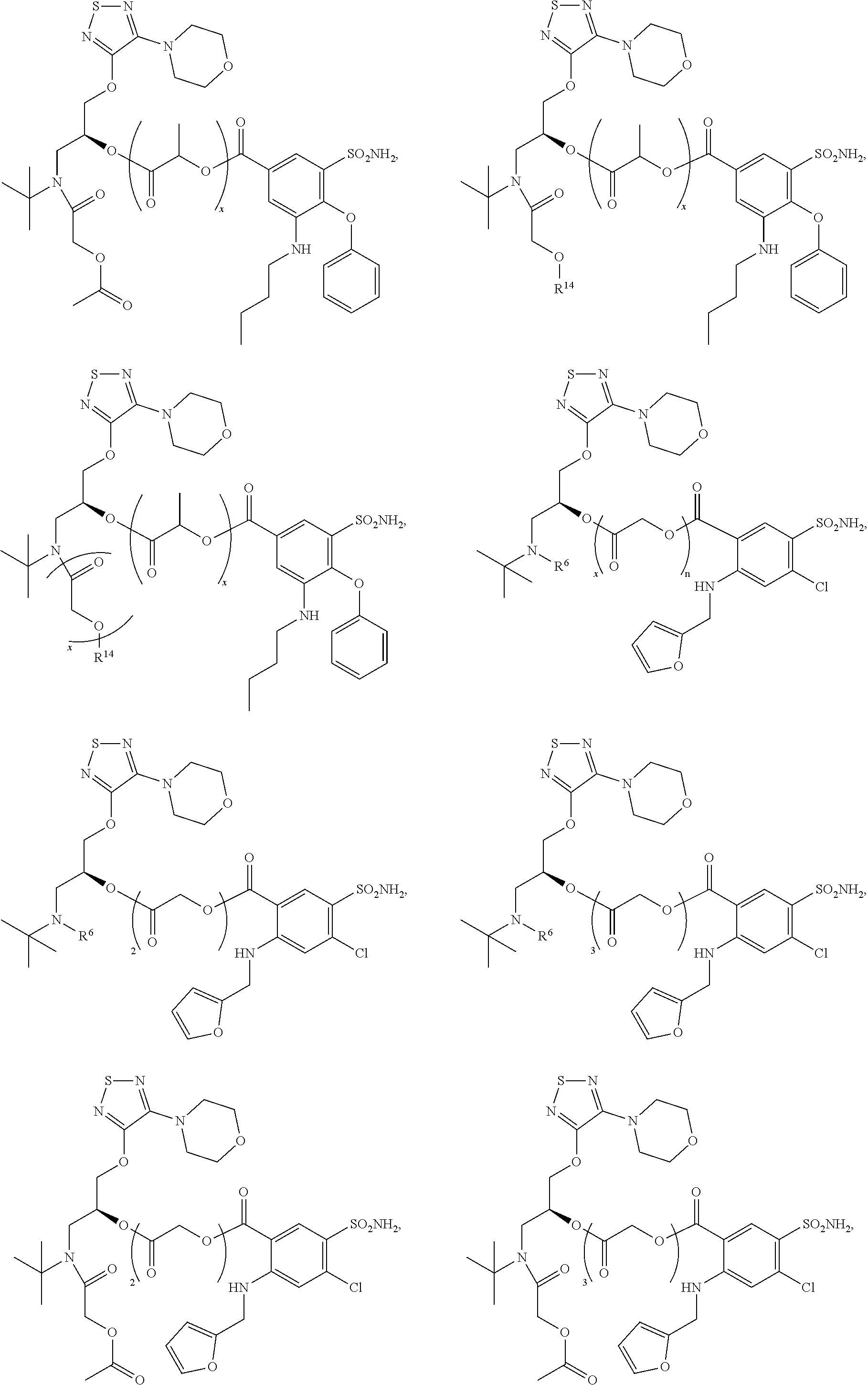

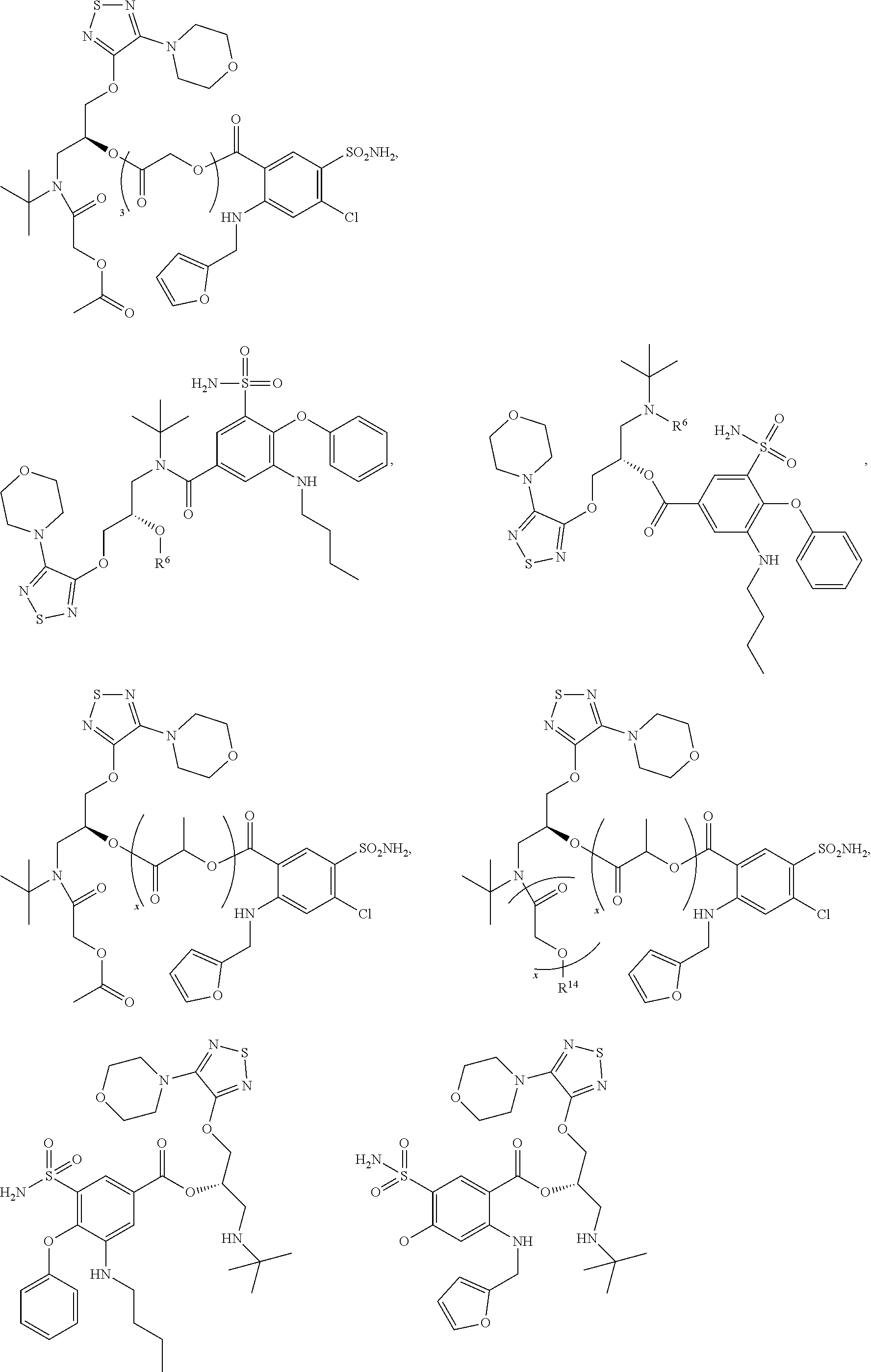

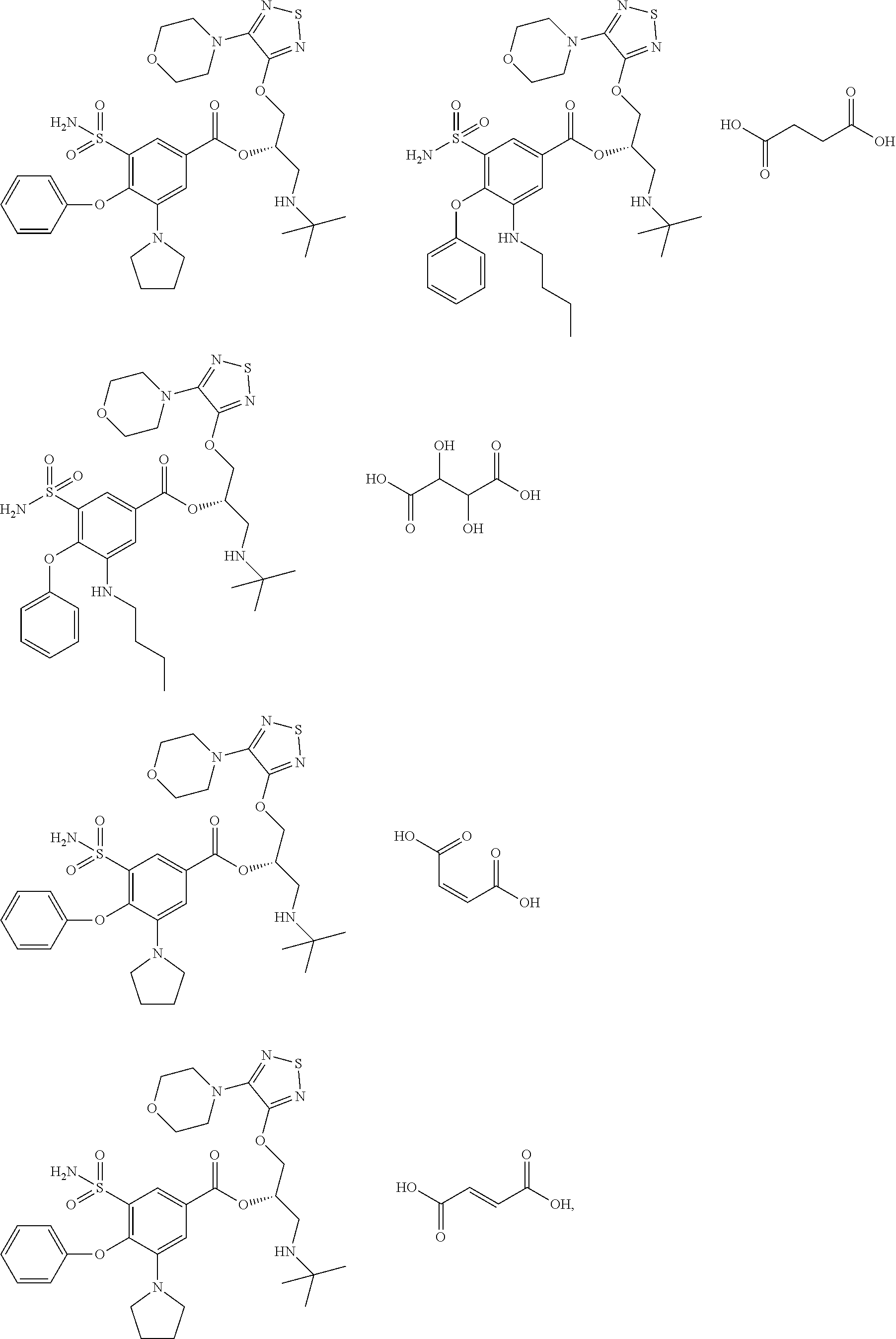

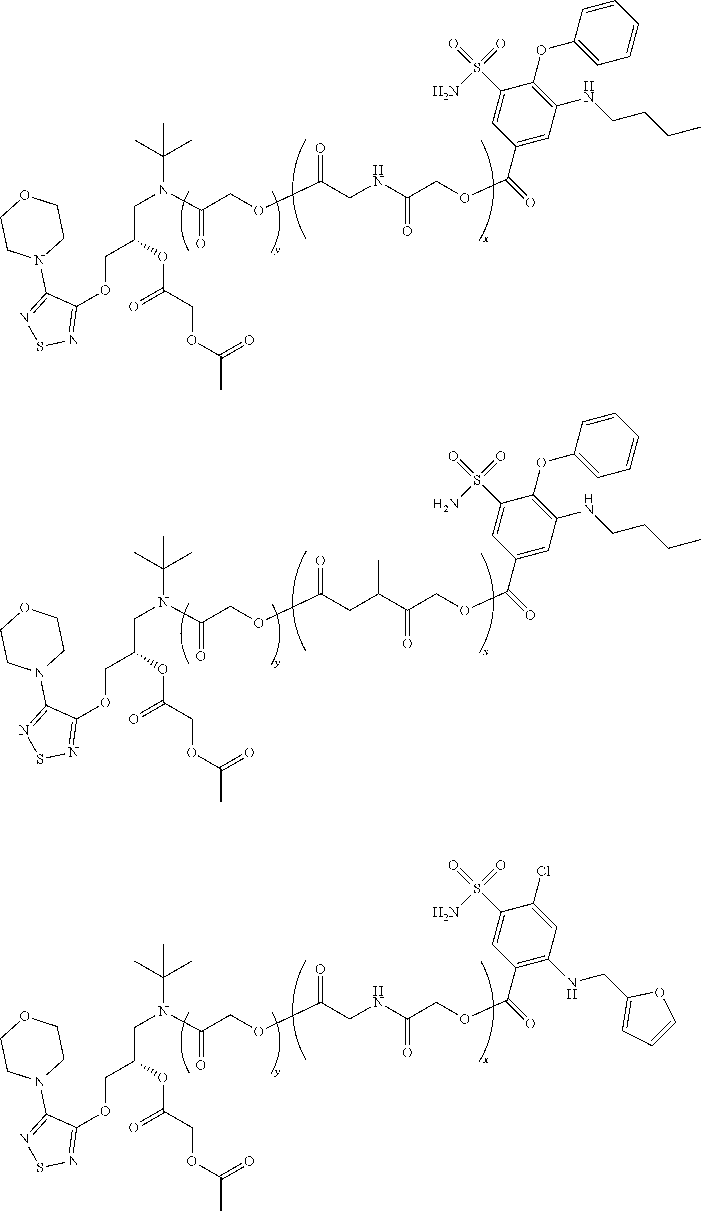

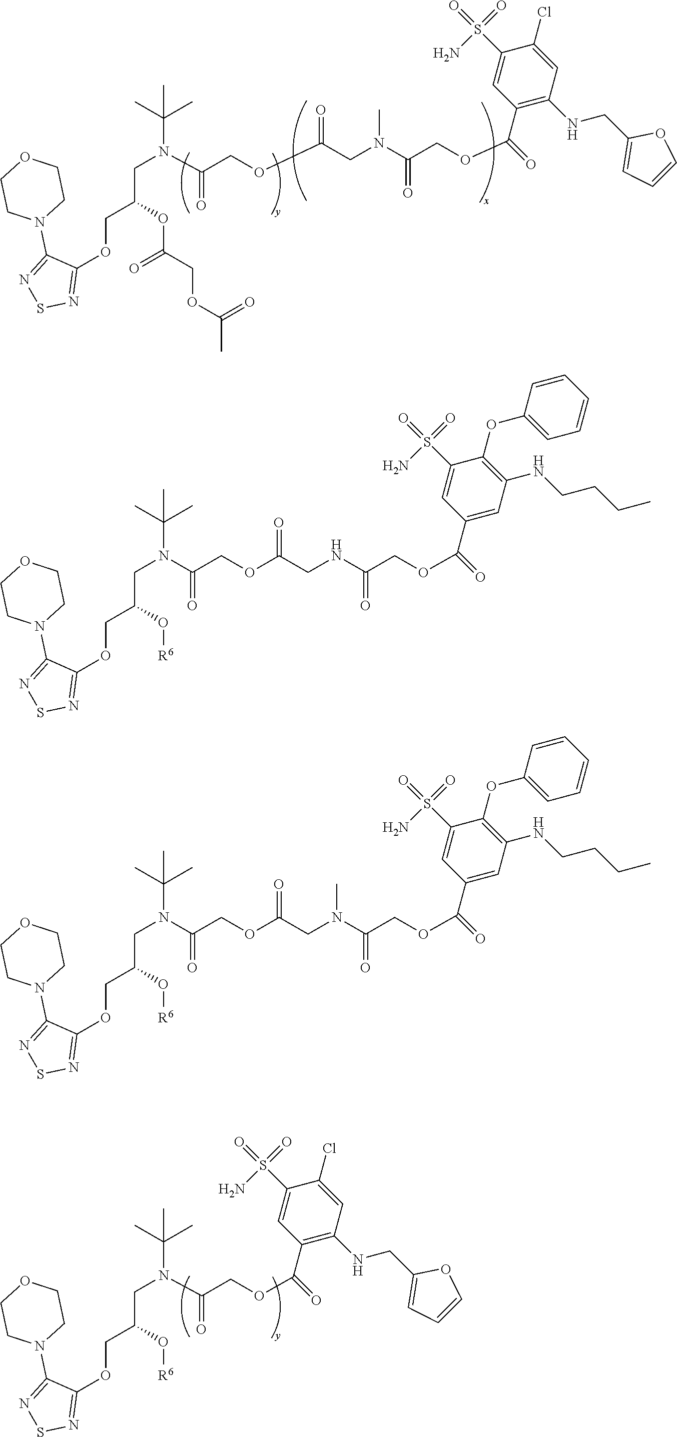

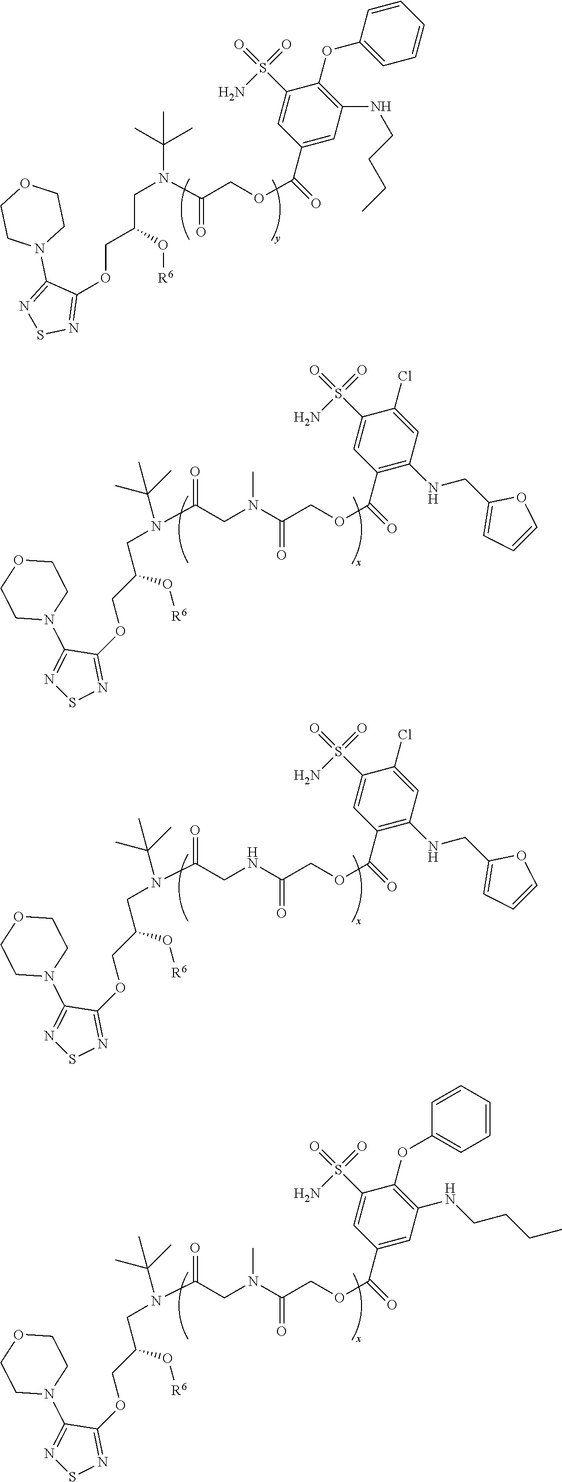

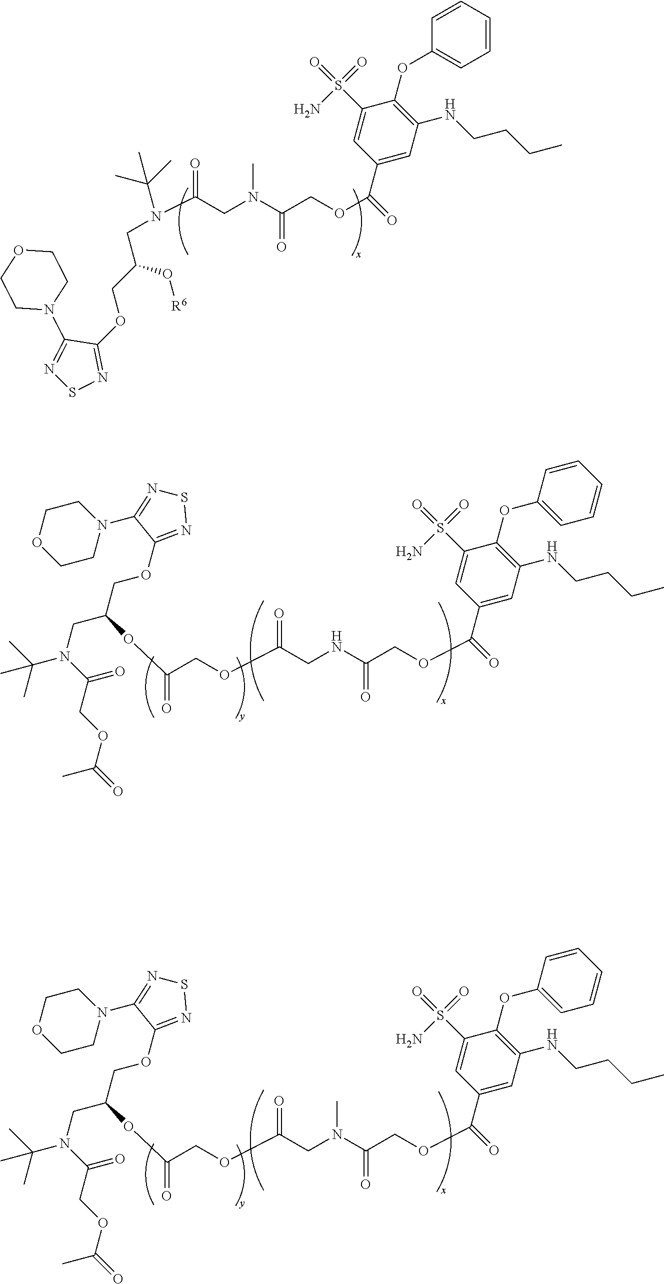

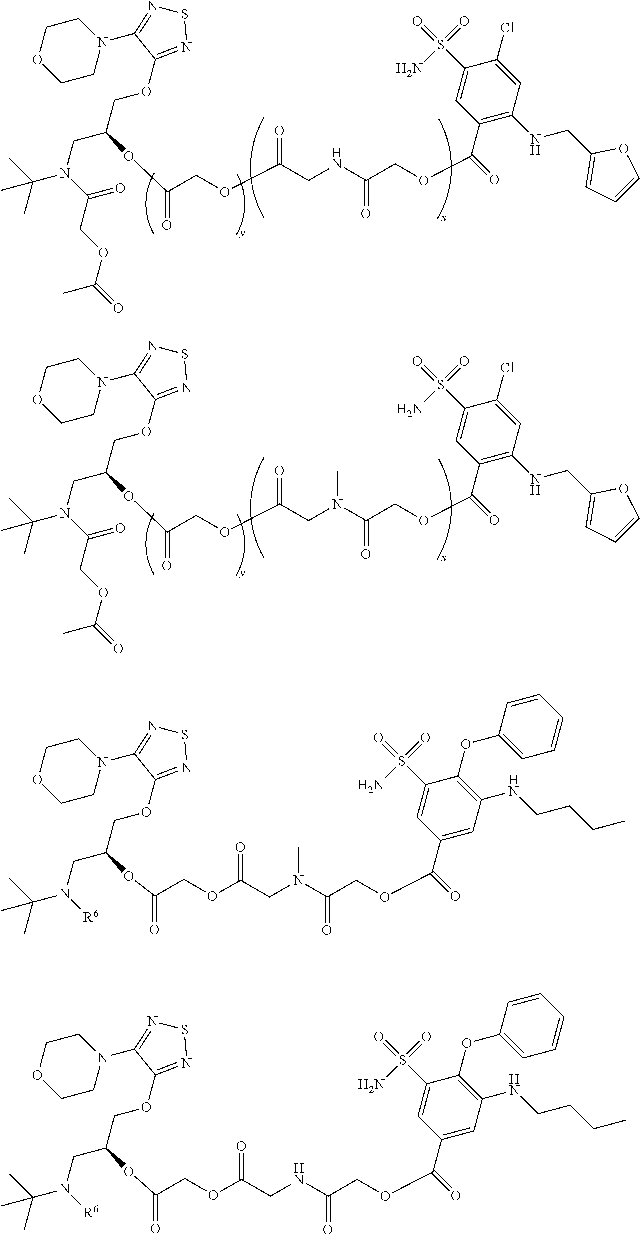

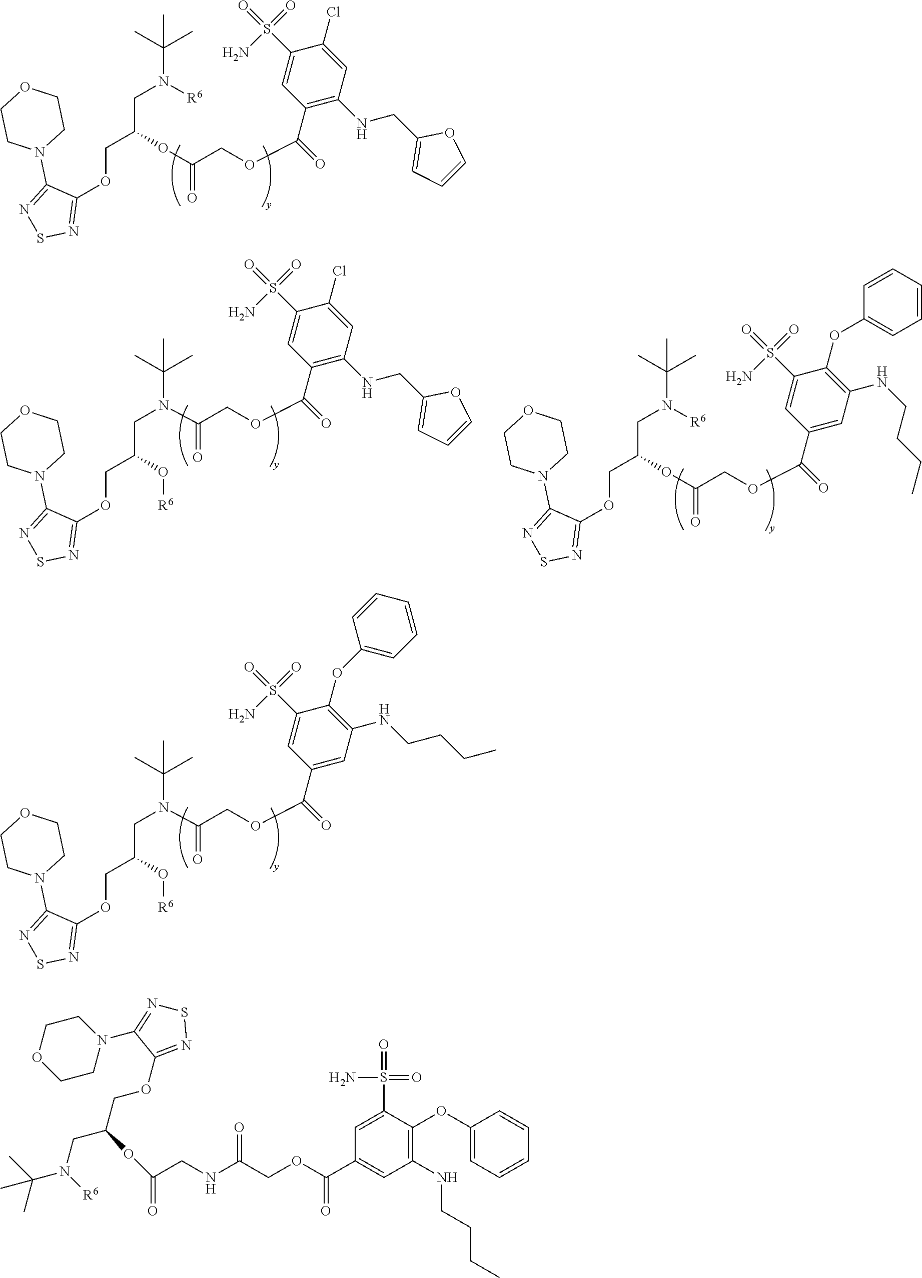

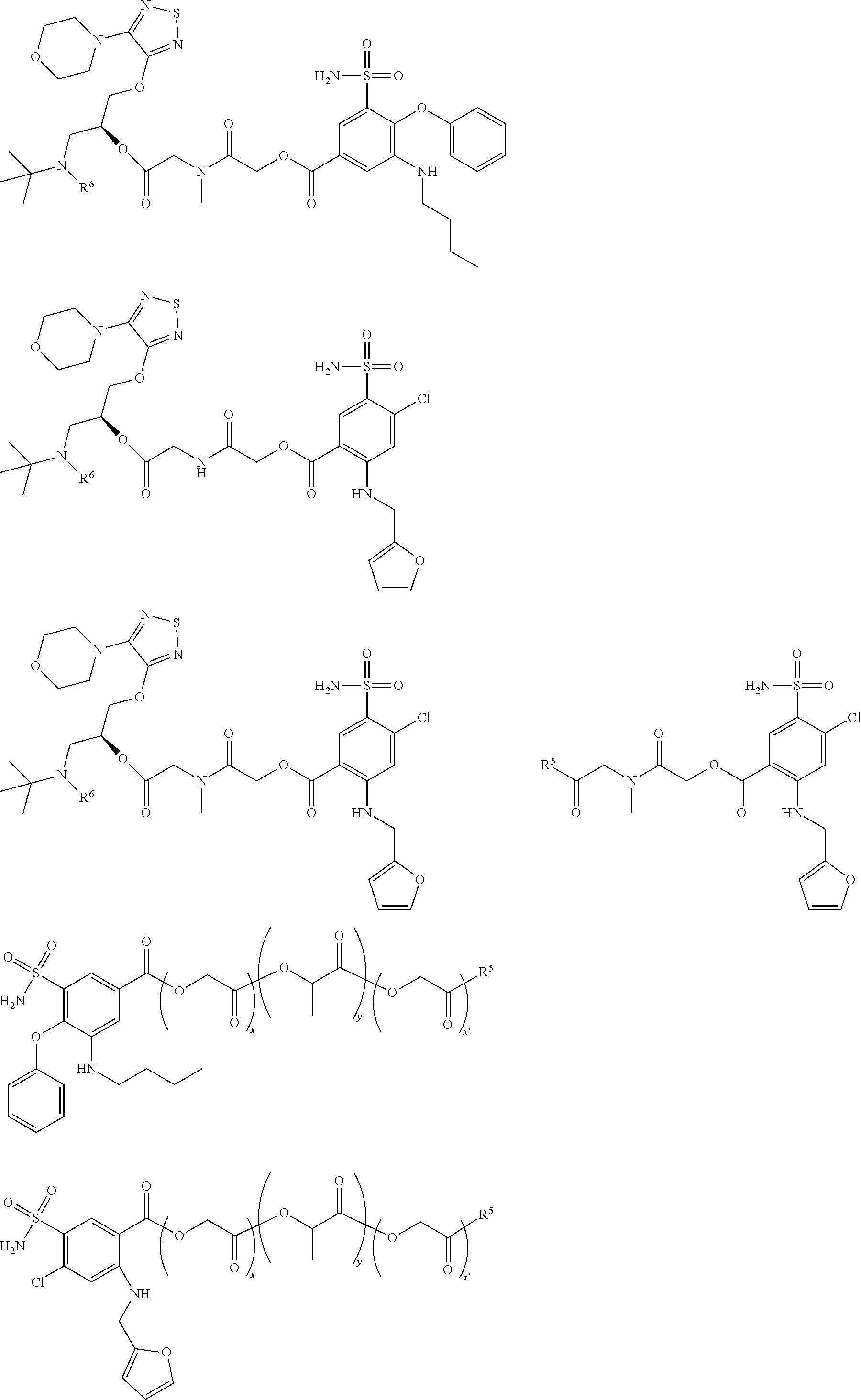

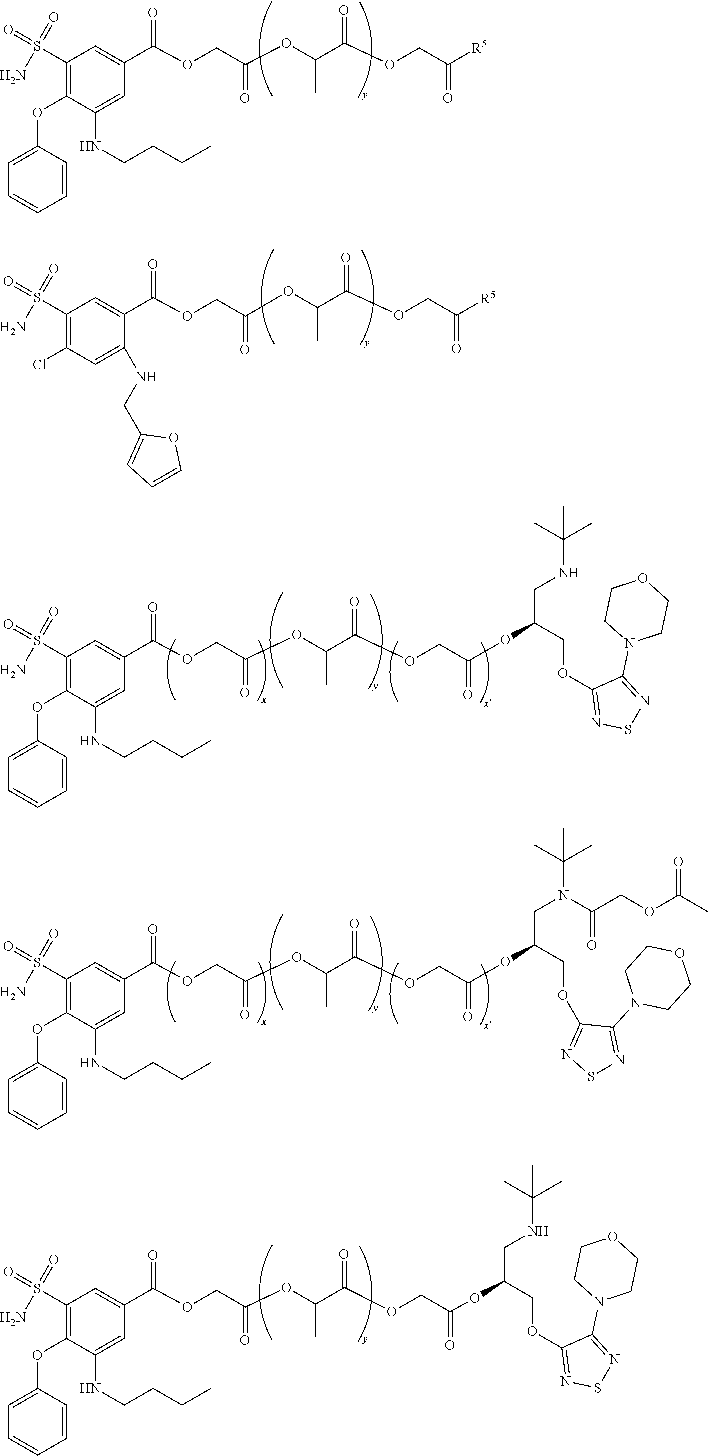

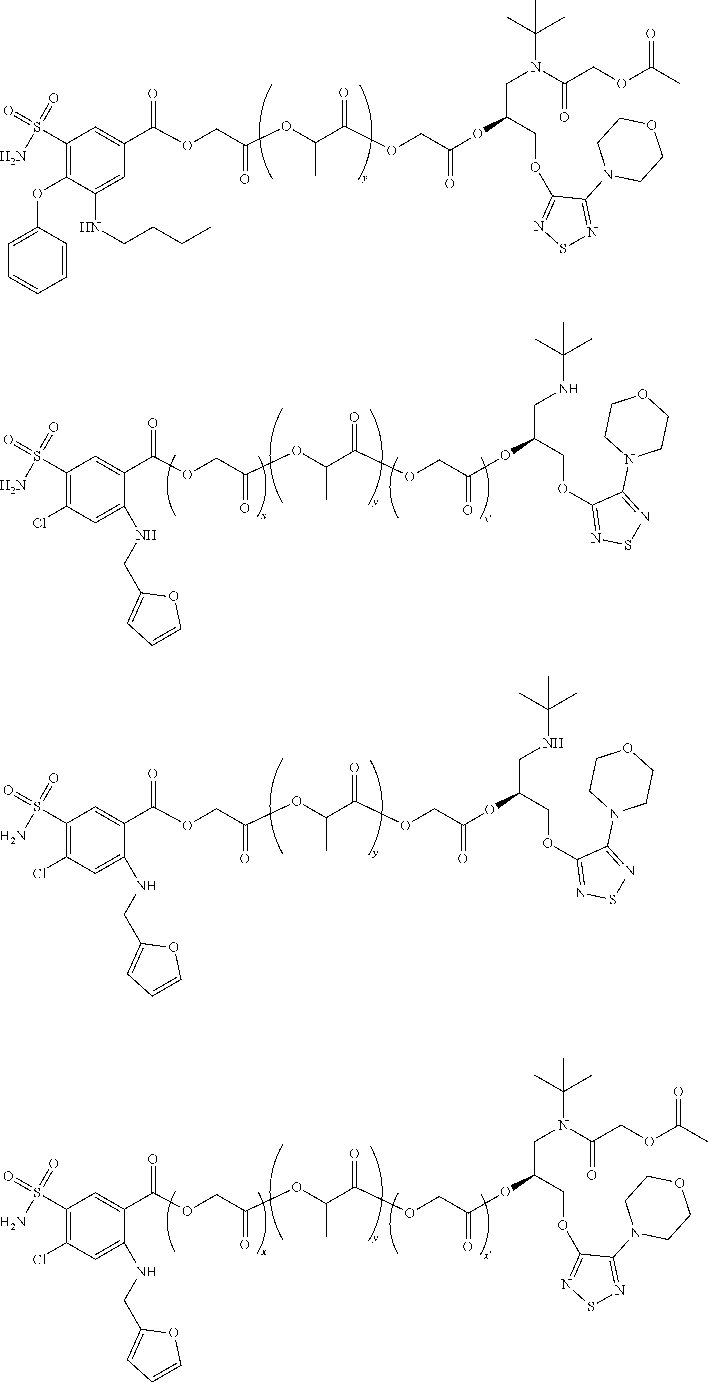

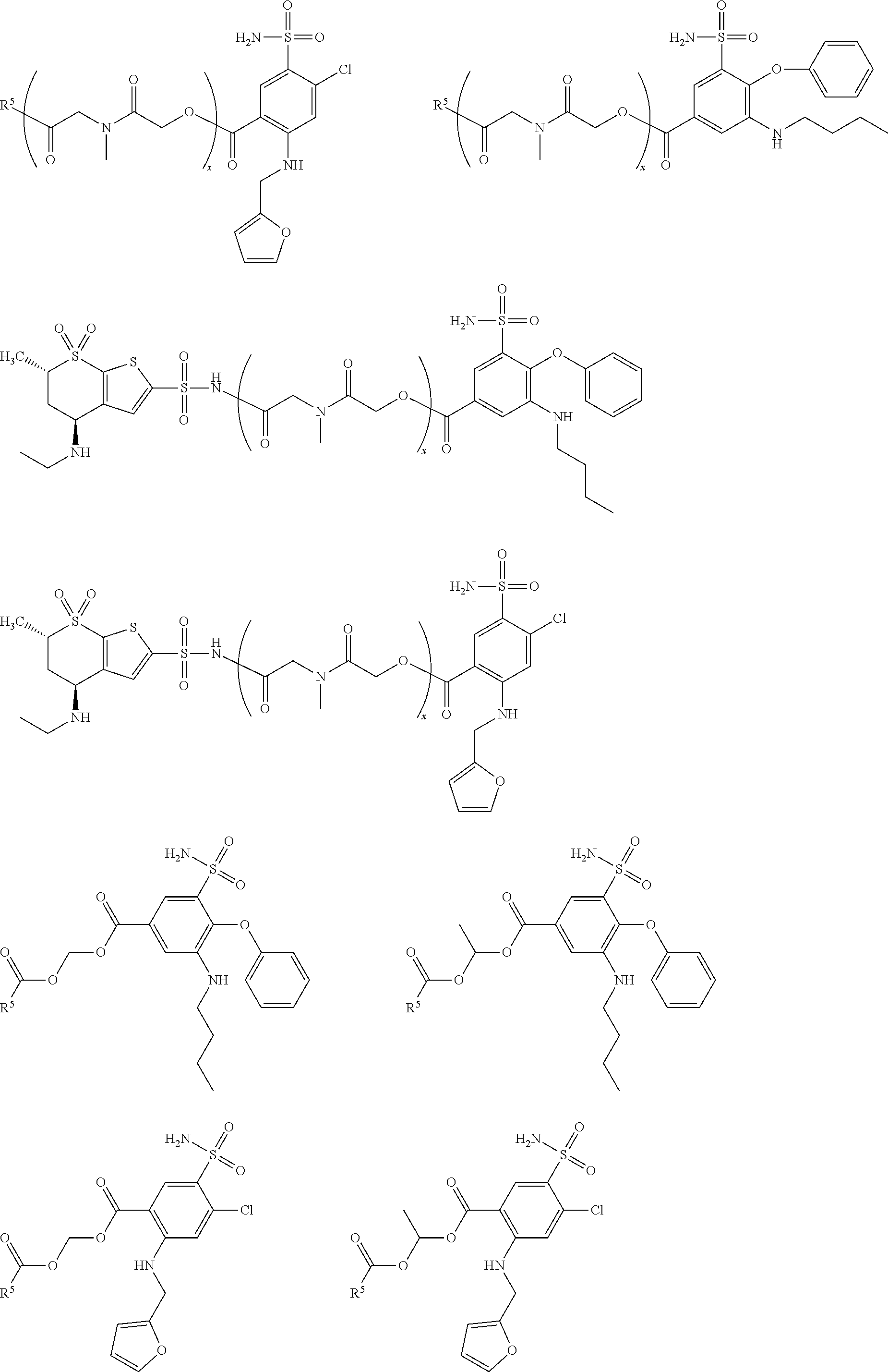

### Example 5. Non-Limiting Examples of Compounds of Formula IX, Formula X, Formula XI, Formula XII, or Formula XII′

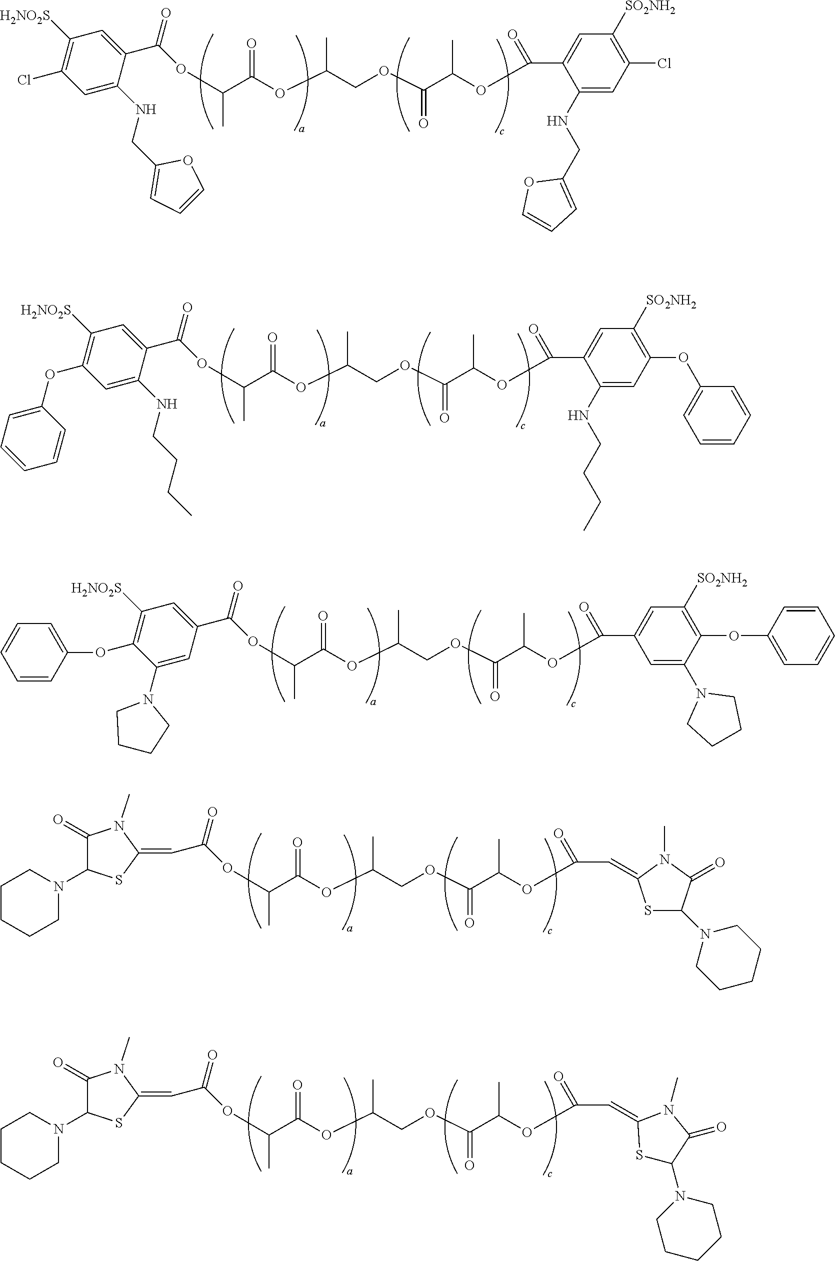

### Embodiments of a and c

In one embodiment a is 1 and c is 1.

In one embodiment a is 1 and c is 2.

In one embodiment a is 1 and c is 3.

In one embodiment a is 1 and c is 4.

In one embodiment a is 1 and c is 5.

In one embodiment a is 1 and c is 6.

In one embodiment a is 1 and c is 7.

In one embodiment a is 1 and c is 8.

In one embodiment a is 2 and c is 1.

In one embodiment a is 2 and y is 2.

In one embodiment a is 2 and c is 3.

In one embodiment a is 2 and c is 4.

In one embodiment a is 2 and c is 5.

In one embodiment a is 2 and c is 6.

In one embodiment a is 2 and c is 7.

In one embodiment a is 2 and c is 8.

In one embodiment a is 3 and c is 1.

In one embodiment a is 3 and c is 2.

In one embodiment a is 3 and c is 3.

In one embodiment a is 3 and c is 4.

In one embodiment a is 3 and c is 5.

In one embodiment a is 3 and c is 6.

In one embodiment a is 3 and c is 7.

In one embodiment a is 3 and c is 8.

In one embodiment a is 4 and c is 1.

In one embodiment a is 4 and c is 2.

In one embodiment a is 4 and c is 3.

In one embodiment a is 4 and c is 4.

In one embodiment a is 4 and c is 5.

In one embodiment a is 4 and c is 6.

In one embodiment a is 4 and c is 7.

In one embodiment a is 4 and c is 8.

In one embodiment a is 5 and c is 1.

In one embodiment a is 5 and c is 2.

In one embodiment a is 5 and c is 3.

In one embodiment a is 5 and c is 4.

In one embodiment a is 5 and c is 5.

In one embodiment a is 5 and c is 6.

In one embodiment a is 5 and c is 7.

In one embodiment a is 5 and c is 8.

In one embodiment a is 6 and c is 1.

In one embodiment a is 6 and c is 2.

In one embodiment a is 6 and c is 3.

In one embodiment a is 6 and c is 4.

In one embodiment a is 6 and c is 5.

In one embodiment a is 6 and c is 6.

In one embodiment a is 6 and c is 7.

In one embodiment a is 6 and c is 8.

In one embodiment a is 7 and c is 1.

In one embodiment a is 7 and y is 2.

In one embodiment a is 7 and y is 3.

In one embodiment a is 7 and c is 4.

In one embodiment a is 7 and c is 5.

In one embodiment a is 7 and c is 6.

In one embodiment a is 7 and c is 7.

In one embodiment a is 7 and c is 8.

In one embodiment a is 8 and c is 1.

In one embodiment a is 8 and c is 2.

In one embodiment a is 8 and c is 3.

In one embodiment a is 8 and c is 4.

In one embodiment a is 8 and c is 5.

In one embodiment a is 8 and c is 6.

In one embodiment a is 8 and c is 7.

In one embodiment a is 8 and c is 8.

### Example 6. Non-Limiting Examples of Compounds of Formula XIII, Formula XIV, Formula XV, Formula XVI, or Formula XVI′

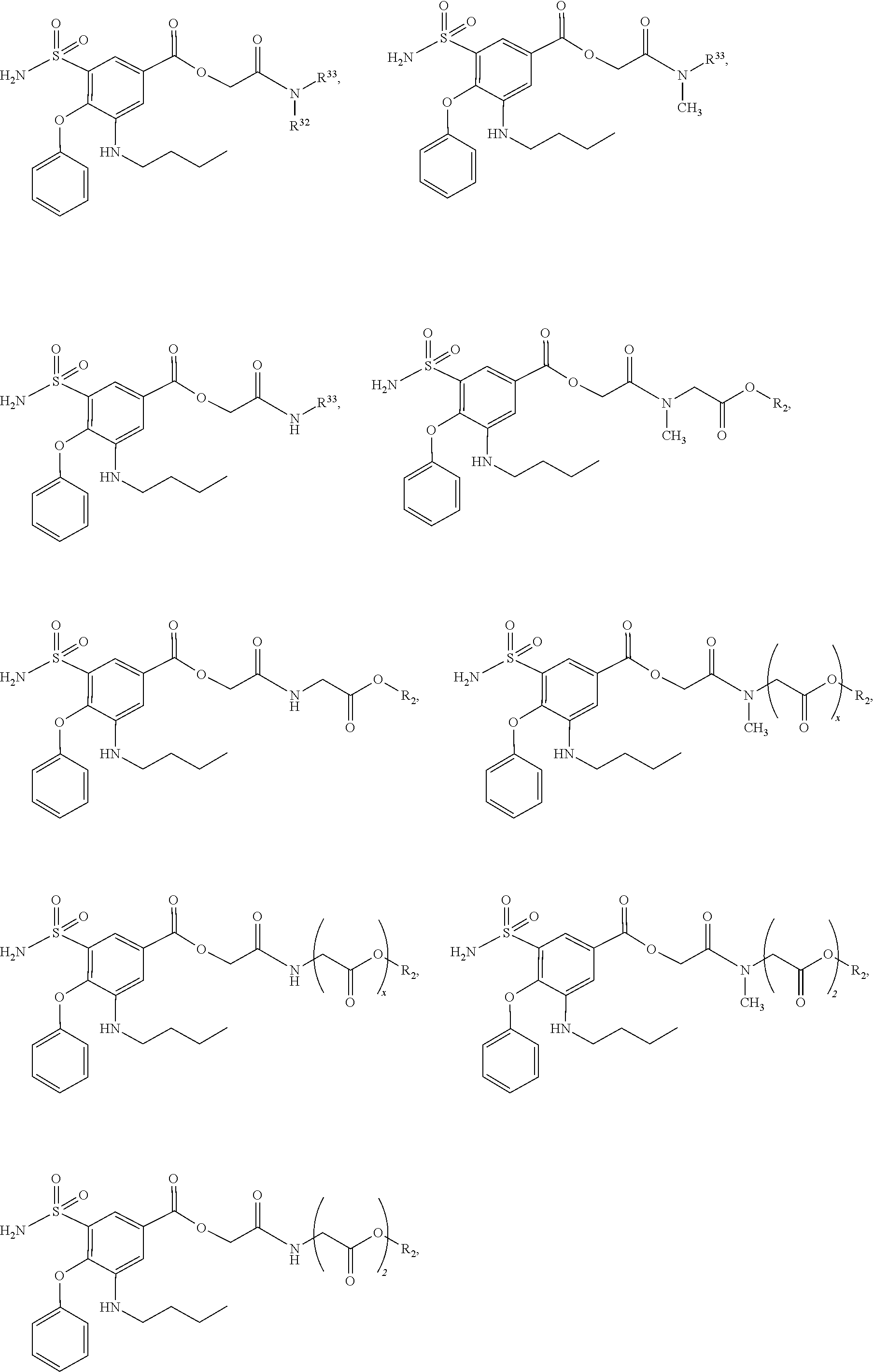

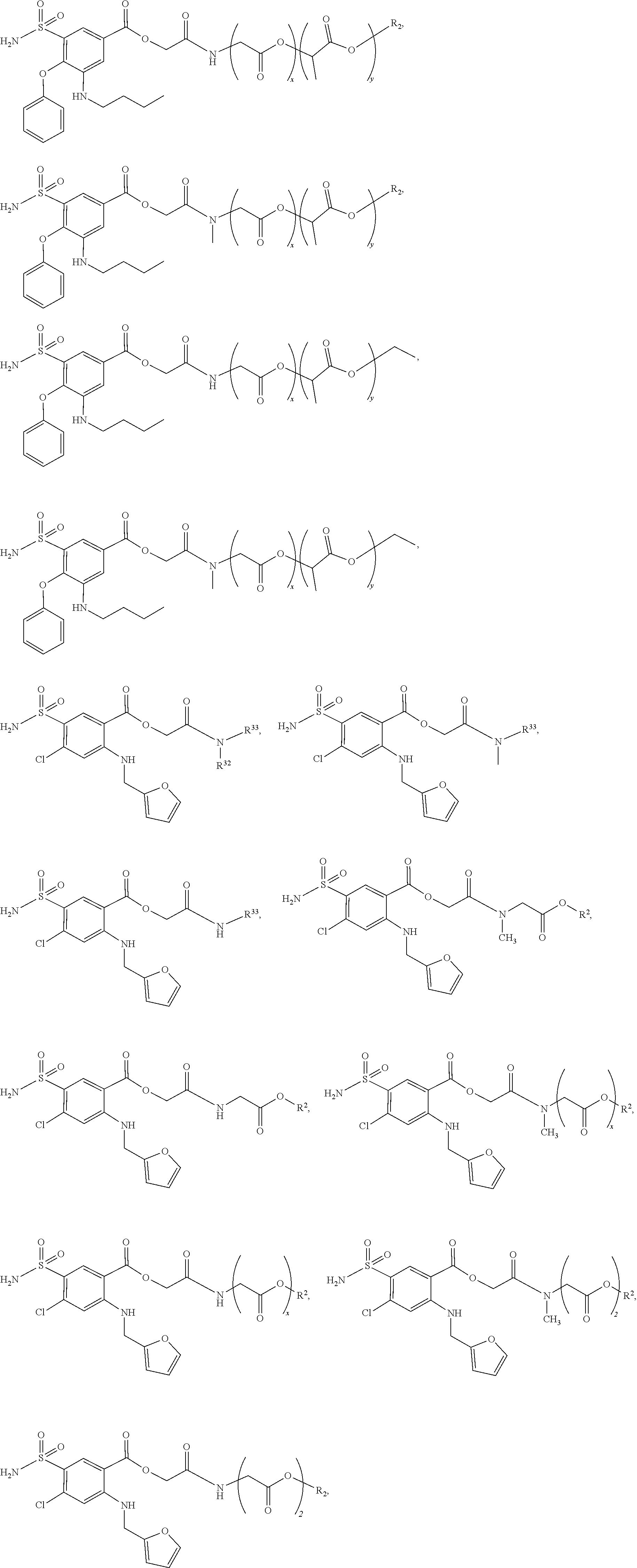

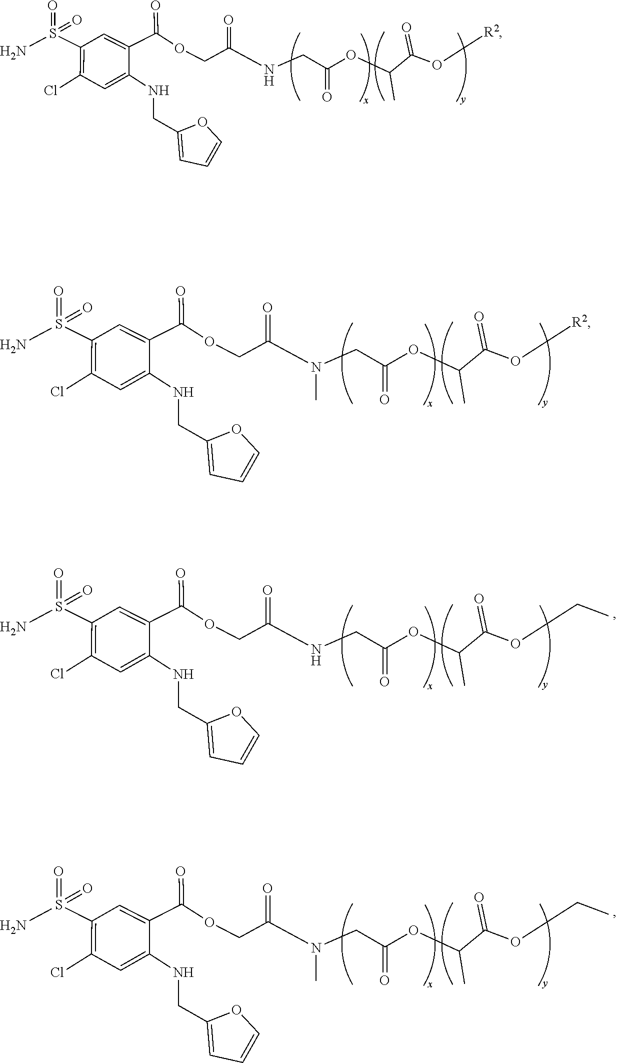

### Example 7. Non-Limiting Examples of Compounds of Formula XVII, Formula XVII, Formula XIX, Formula XX, and Formula XX′

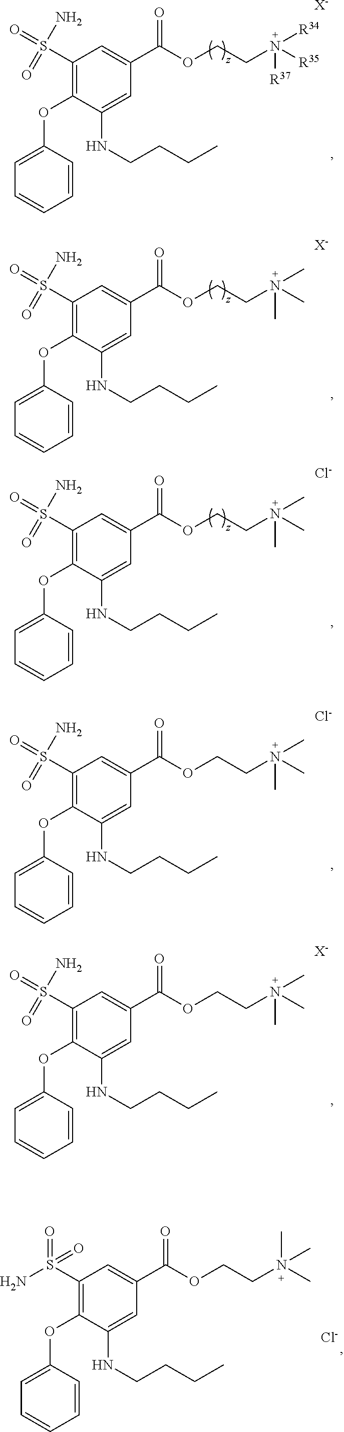

### Example 8. Non-Limiting Examples of Compounds of Formula XXI, Formula XXII, Formula XXIII, Formula XXIV, or Formula XXIV′

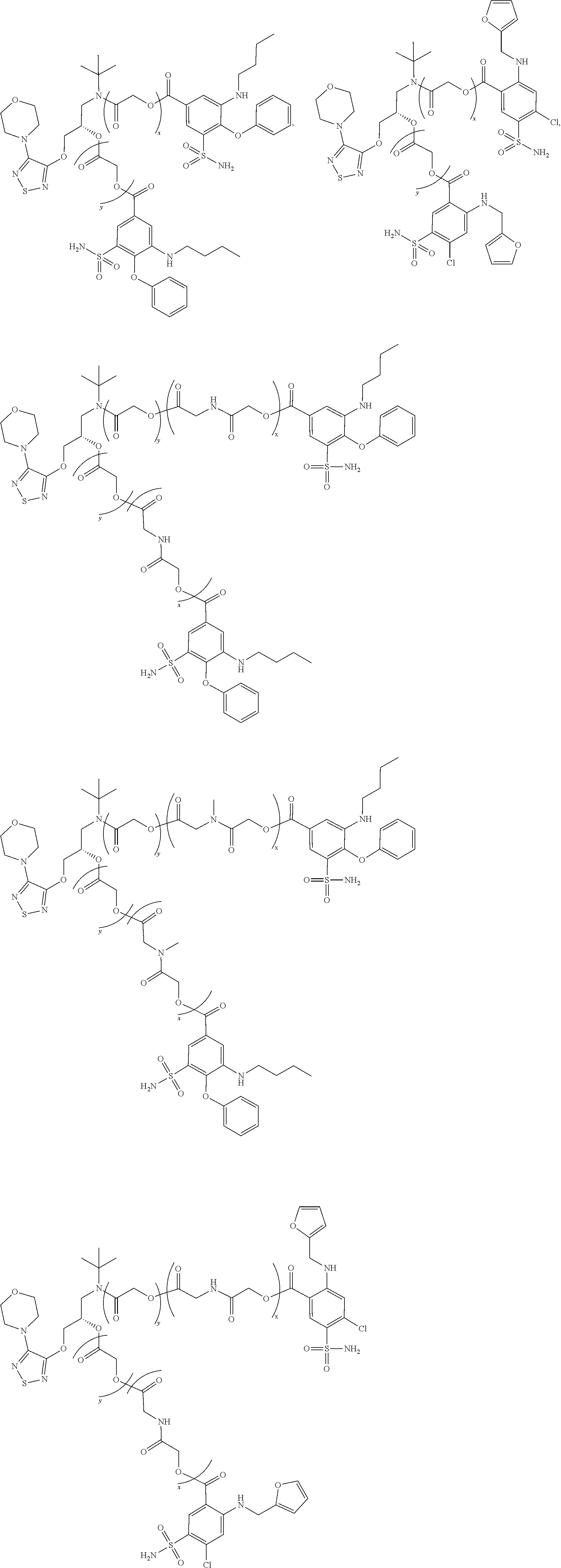

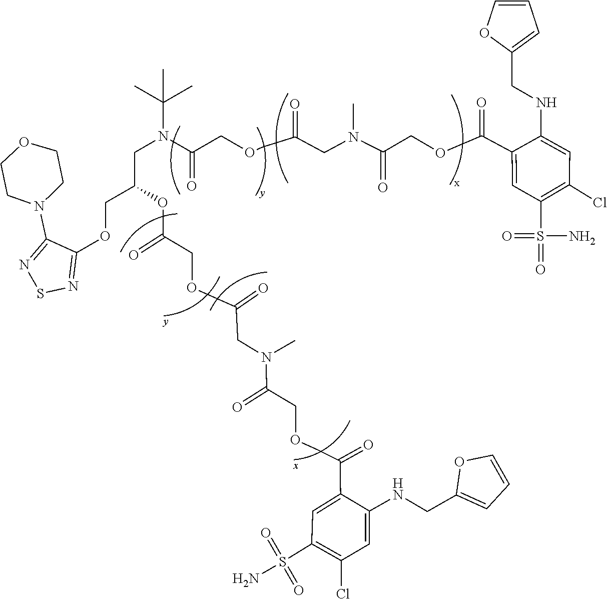

### Example 9. Synthesis of PLA-Linkers

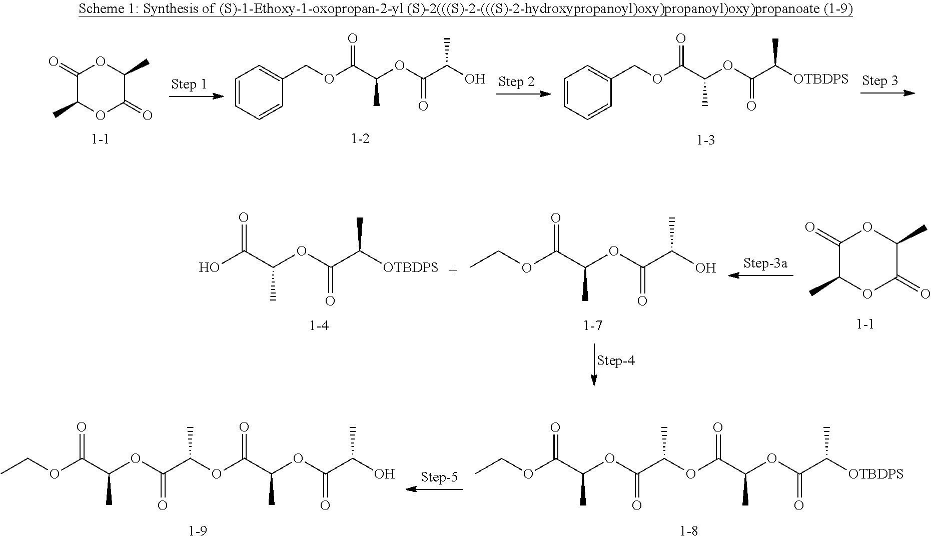

Step 1: Preparation of (S)-2-Hydroxy-propionic acid (S)-1-benzyloxycarbonyl-ethyl ester (1-2): To a solution of (3S,6S)-3,6-dimethyl-[1,4]dioxane-2,5-dione 1-1 (5.0 g, 34.72 mmol) in toluene (100 mL) was added benzyl alcohol (3.2 mL, 31.72 mmol) and camphor sulfonic acid (0.8 g, 3.47 mmol) at 25-30° C. The reaction mixture was allowed to stir at 80° C. over a period of 2 hours. The resulting reaction mixture was diluted with ethyl acetate (800 mL) and washed with water (2×400 mL). The crude product obtained upon evaporation of volatiles was purified through preparative HPLC to obtain product 1-2 as a pale yellow liquid 5.5 g (63%).

Step 2: Preparation of (S)-2-(tert-Butyl-diphenyl-silanyloxy)-propionic acid (S)-1-benzyloxycarbonyl-ethyl ester (1-3): To a solution of (S)-2-hydroxy-propionic acid (S)-1-benzyloxycarbonyl-ethyl ester 1-2 (0.1 g, 0.23 mmol) in dichloromethane (2 mL) was added triethylamine (0.23 mL, 1.61 mmol), TBDPS-Cl (0.43 mL, 1.618 mmol) and a catalytic amount of 4-dimethylaminopyridine at 0° C. The reaction mixture was stirred at room temperature over period of 8 hours. The resulting reaction mixture was quenched with water (20 mL) and extracted with ethyl acetate (2×50 mL). The volatiles were evaporated under reduced pressure to obtain product 1-3 as a colorless liquid 200 mg (74%).

Step 3: Preparation of (S)-2-(tert-Butyl-diphenyl-silanyloxy)-propionic acid (S)-1-carboxy-ethyl ester (1-4): (S)-2-(tert-butyl-Diphenyl-silanyloxy)-propionic acid (S)-1-benzyloxycarbonyl-ethyl ester 1-3 (1.5 g), methanol (20 mL) and 10% Pd/C (0.3 g, 50% wet) were taken in a 100 mL autoclave vessel. The reaction mixture was stirred at 25-30° C. under hydrogen pressure (5 kg/cm²) over a period of 2 hours. After completion of the reaction, the reaction mixture was filtered through celite bed and concentrated under reduced pressure. The crude product obtained upon evaporation of volatiles was purified through silica gel (60-120 mesh) column chromatography (10% methanol in dichloromethane) to afford 1-4 as a colorless liquid 700 mg (58%).

Step 3a: Preparation of (S)-2-Hydroxy-propionic acid (S)-1-ethoxycarbonyl-ethyl ester (1-5): To a solution of (3S,6S)-3,6-dimethyl-[1,4]dioxane-2,5-dione 1-1 (5.0 g, 34.72 mmol) in toluene (100 mL) was added ethanol (1.92 mL, 31.98 mmol) and camphor sulfonic acid (0.8 g, 3.47 mmol) at 25-30° C. The reaction mixture was allowed to stir at 80° C. over a period of 2 hours. The resulting reaction mixture was diluted with ethyl acetate (800 mL) and washed with water (2×200 mL). The crude product obtained upon evaporation of volatiles was purified through silica gel (230-400 mesh) column chromatography (13% ethyl acetate in hexane) to obtain product 1-7 as a colorless liquid 6.6 g (60%).

Step 4: Preparation of (S)-2-(tert-Butyl-diphenyl-silanyloxy)-propionic acid (S)-1-[(S)-1-((S)-1-ethoxycarbonyl-ethoxycarbonyl)-ethoxycarbonyl]-ethyl ester (2-1): To a solution of (S)-2-(tert-butyl-diphenyl-silanyloxy)-propionic acid (S)-1-carboxy-ethyl ester 1-7 (5.473 g, 13.68 mmol) in dichloromethane (60 mL), was added EDC.HCl (3.014 g, 15.78 mmol), (S)-2-Hydroxy-propionic acid (S)-1-ethoxycarbonyl-ethyl ester 1-5 (2 μg, 10.52 μmmol) and 4-dimethylaminopyridine (128 mg, 1.05 mmol) at 0° C. The reaction mixture was allowed to stir at 25-30° C. over a period of 1 hour. The resulting reaction mass was quenched with water (200 mL), extracted with dichloromethane (250×3 mL), dried over Na₂SO₄ and concentrated under reduced pressure. The crude product obtained upon evaporation of volatiles was purified through silica gel (230-400 mesh) column chromatography (3% ethyl acetate in hexane) to obtain product 2-1 as a colorless liquid 4.2 g (70%).

Step 5: Preparation of (S)-2-Hydroxy-propionic acid (S)-1-[(S)-1-((S)-1-ethoxycarbonyl-ethoxycarbonyl)-ethoxycarbonyl]-ethyl ester (1-9): To a solution of (S)-2-(tert-Butyl-diphenyl-silanyloxy)-propionic acid (S)-1-[(S)-1-((S)-1-ethoxycarbonyl-ethoxycarbonyl)-ethoxycarbonyl]-ethyl ester 1-8 (4 g, 6.99 mmol) in tetrahydrofuran (40 mL) were added tetra butyl ammonium fluoride (10.49 mL, 1.0M, 10.49 mmol) and acetic acid (0.63 g, 10.49 mmol) at 0° C. The reaction mixture was allowed to stir at room temperature over a period of 1 hour. The resulting reaction mixture was concentrated under reduced pressure and the crude product was obtained upon evaporation of the volatiles. The crude product was purified through silica gel (230-400 mesh) column chromatography (12% ethyl acetate in hexane) to afford product 1-9 as a colourless liquid 1.0 g (43%).

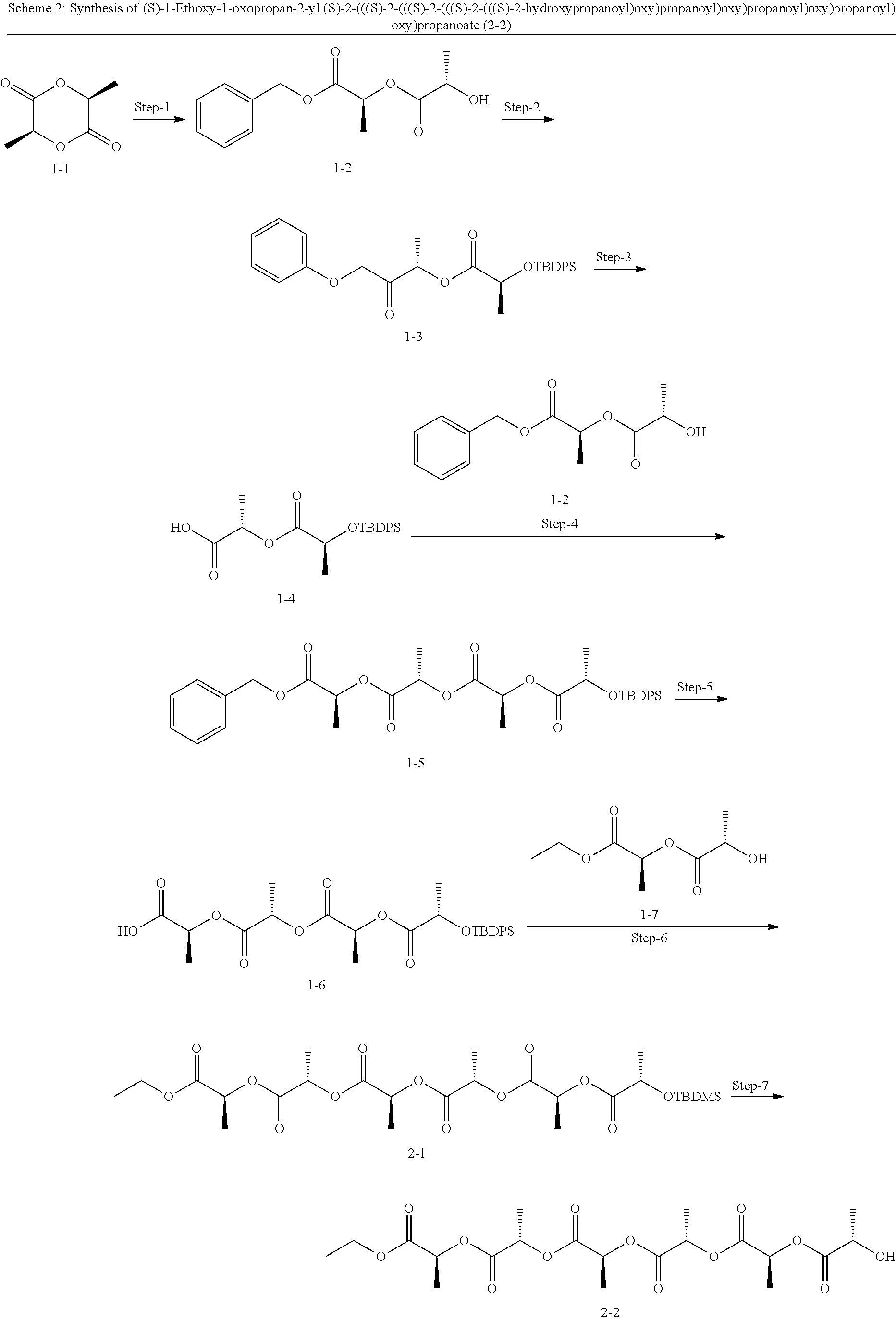

Step 1: Preparation of (S)-2-Hydroxy-propionic acid (S)-1-benzyloxycarbonyl-ethyl ester (1-2): To a solution of (3S,6S)-3,6-dimethyl-[1,4]dioxane-2,5-dione 1-1 (5.0 g, 34.72 mmol) in toluene (100 mL) was added benzyl alcohol (3.2 mL, 31.72 mmol) and camphor sulfonic acid (0.8 g, 3.47 mmol) at 25-30° C. The reaction mixture was allowed to stir at 80° C. over a period of 2 h. The resulting reaction mixture was diluted with ethyl acetate (800 mL) and washed with water (2×400 mL). The crude product obtained upon evaporation of volatiles was purified through preparative HPLC to obtain product 2-2 as a pale yellow liquid 5.5 g (63%). ¹H NMR (400 MHz, DMSO-d₆) δ 7.41-7.32 (m, 5H), 5.48 (d, J=5.6 Hz, 1H), 5.15 (s, 2H), 5.10 (q, J=7 Hz, 1H), 4.20-4.18 (m, 1H), 1.42 (d, J=7 Hz, 3H), 1.16 (d, J=7 Hz, 3H). MS m/z [M+H]⁺ 253.4, [M+NH₄⁺]⁺ 270.3.

Step 2: Preparation of (S)-2-(tert-Butyl-diphenyl-silanyloxy)-propionic acid (S)-1 benzyloxycarbonyl-ethyl ester (1-3): To a solution of (S)-2-hydroxy-propionic acid (S)-1-benzyloxycarbonyl-ethyl ester 1-2 (0.1 g, 0.23 mmol) in dichloromethane (5 mL) were added triethylamine (0.23 mL, 1.61 mmol), TBDPS-Cl (0.43 mL, 1.618 mmol) and catalytic amount of 4-dimethylaminopyridine at 0° C. The reaction mixture was stirred at room temperature over period of 8 h. The resulting reaction mixture was quenched with water (20 mL) and extracted with ethyl acetate (2×50 mL). Then volatiles were evaporated under reduced pressure to obtain product 1-3 as a colorless liquid 200 mg (74%). This material was carried into the next step without further purification.

Step 3: Preparation of (S)-2-(tert-Butyl-diphenyl-silanyloxy)-propionic acid (S)-1-carboxy-ethyl ester (1-4): To a 100 mL autoclave vessel were added a solution of (S)-2-(tert-butyl-diphenyl-silanyloxy)-propionic acid (S)-1-benzyloxycarbonyl-ethyl ester 1-3 (1.5 g) in methanol (20 mL) and 10% Pd/C (0.3 g, 50% wet) at 25-30° C. The reaction mixture was stirred at room temperature under hydrogen pressure (5 kg/cm²) over a period of 2 h. After completion of the reaction, the reaction mixture was filtered through a celite bed and concentrated under reduced pressure. The crude product obtained upon evaporation of volatiles was purified through silica gel (60-120 mesh) column chromatography (10% methanol in dichloromethane) to obtain product 3-4 as a colorless liquid 700 mg (58%). ¹H-NMR (400 MHz, DMSO-d₆) δ 13.1 (bs, 1H), 7.63-7.62 (m, 4H), 7.62-7.37 (m, 6H), 4.77 (q, J=7.6 Hz, 1H), 4.26 (q, J=8.0.0 Hz, 1H), 1.31 (d, J=6.8 Hz, 3H), 1.23 (d, J=7.2 Hz, 3H), 1.02 (s, 9H); MS m/z [M−H]⁻ 399.1.

Step 4: Preparation of (S)-2-(tert-Butyl-diphenyl-silanyloxy)-propionic acid (S)-1-[(S)-1-((S)-1-benzyloxycarbonyl-ethoxycarbonyl)-ethoxycarbonyl]-ethyl ester (1-5): To a solution of (S)-2-hydroxy-propionic acid (S)-1-benzyloxycarbonyl-ethyl ester 1-2 (6.0 g, 33.2 mmol) and (S)-2-(tert-butyl-diphenyl-silanyloxy)-propionic acid (S)-1-carboxy-ethyl ester 1-4 (17.3 g, 7.77 mmol) in dichloromethane (60 mL) were added EDC.HCl (8.2 g, 43.2 mmol), 4-dimethylaminopyridine (405 mg, 3.3 mmol) at 0° C. The reaction mixture was allowed to stir at 25-30° C. over a period of 1 h. The resulting reaction mass was quenched with water (200 mL), extracted with dichloromethane (3×250 mL), dried over Na₂SO₄ and concentrated under reduced pressure. The crude product obtained upon evaporation of volatiles was purified through silica gel (230-400 mesh) column chromatography (10% methanol in dichloromethane) to obtain product 1-5 as a pale yellow liquid 5.8 g (94%). ¹H NMR (400 MHz, DMSO-d₆) δ 7.60 (d, J=8 Hz, 4H), 7.49-7.33 (m, 11H), 5.20-5.15 (m, 4H), 4.95 (q, J=7.2 Hz, 1H), 4.29 (q, J=6.4 Hz, 1H), 1.43 (d, J=7.2 Hz, 3H), 1.39 (d, J=7.2 Hz, 3H), 1.31 (d, J=6.8 Hz, 3H), 1.28 (d, J=1.28 Hz, 3H), 1.02 (s, 9H); MS m/z [M+NH₄]⁺ 652.8.

Step 5: Preparation of (S)-2-(tert-Butyl-diphenyl-silanyloxy)-propionic acid (S)-1-[(S)-1-((S)-1-carboxy-ethoxycarbonyl)-ethoxycarbonyl]-ethyl ester (1-6): To a 100 mL autoclave vessel were added a solution of (S)-2-(tert-Butyl-diphenyl-silanyloxy)-propionic acid (S)-1-[(S)-1-((S)-1-benzyloxycarbonyl-ethoxycarbonyl)-ethoxycarbonyl]-ethyl ester 1-5 (700 mg, 1.10 mmol) in methanol (10 mL) and 10% Pd/C (140 mg, 50% wet) at 25-30° C. The reaction mixture was stirred at room temperature under hydrogen pressure (5 kg/cm²) over a period of 2 h. After completion of the reaction, the reaction mixture was filtered through a celite bed and concentrated under reduced pressure. The crude product obtained upon evaporation of volatiles was purified through silica gel (60-120 mesh) column chromatography (10% methanol in dichloromethane) to obtain product 1-6 as a pale yellow liquid 420 mg (78%). ¹H NMR (400 MHz, DMSO-d₆) δ 13.2 (bs, 1H), 7.62-7.60 (m, 4H), 7.59-7.40 (m, 6H), 5.16 (q, J=7.2 Hz 1H), 4.98-4.93 (m, 2H), 4.29 (q, J=6.8, 1H), 1.44 (d, J=7.2 Hz, 3H), 1.40 (d, J=7.2 Hz, 3H), 1.31-1.30 (m, 6H), 1.01 (s, 9H); MS m/z [M+NH₄]⁺ 562.3; MS m/z [M−H]⁻ 543.1.

Step 6: Preparation of (S)-2-(tert-Butyl-diphenyl-silanyloxy)-propionic acid (S)-1-((S)-1-{(S)-1-[(S)-1-((S)-1-ethoxycarbonyl-ethoxycarbonyl)-ethoxycarbonyl]-ethoxycarbonyl}-ethoxycarbonyl)-ethyl ester (1-8): To a solution of (S)-2-(tert-Butyl-diphenyl-silanyloxy)-propionic acid (S)-1-[(S)-1-((S)-1-carboxy-ethoxycarbonyl)-ethoxycarbonyl]-ethyl ester 1-6 (7.44 g, 13.68 mmol) in dichloromethane (20 mL) were added EDC.HCl (2.411 g, 12.62 mmol), (S)-2-Hydroxy-propionic acid (S)-1-ethoxycarbonyl-ethyl ester (2 g, 10.52 mmol) 1-7 and 4-dimethylaminopyridine (128 mg, 1.05 mmol) at 0° C. The reaction mixture was allowed to stir at 25-30° C. over a period of 1 h. The resulting reaction mass was quenched with water (200 mL), extracted with dichloromethane (2×250 mL), dried over Na₂SO₄ and concentrated under reduced pressure. The crude product obtained upon evaporation of volatiles was purified through silica gel (230-400 mesh) column chromatography (5% ethyl acetate in hexane) to obtain product 1-8 as a colorless liquid 6.0 g (79%). ¹H NMR (400 MHz, DMSO-d₆) δ 7.63-7.57 (m, 4H), 7.51-7.36 (m, 6H), 5.23-5.15 (m, 3H), 5.08 (q, J=7 Hz, 1H), 4.95 (q, J=7 Hz, 1H), 4.28 (q, J=7 Hz, 1H), 4.16-4.06 (m, 2H), 1.50-1.39 (m, 12H), 1.34-1.25 (m, 6H), 1.18 (t, 3H), 1.02 (s, 9H); MS m/z [M+NH₄]⁺ 735.0.

Step 7: Preparation of (S)-2-Hydroxy-propionic acid (S)-1-((S)-1-{(S)-1-[(S)-1-((S)-1-ethoxycarbonyl-ethoxycarbonyl)-ethoxycarbonyl]-ethoxycarbonyl}-ethoxycarbonyl)-ethyl ester (2-2): To a solution of (S)-2-(tert-Butyl-diphenyl-silanyloxy)-propionic acid (S)-1-((S)-1-{(S)-1-[(S)-1-((S)-1-ethoxycarbonyl-ethoxycarbonyl)-ethoxycarbonyl]-ethoxycarbonyl}-ethoxycarbonyl)-ethyl ester 2-1 (7 g, 9.78 mmol) in tetrahydrofuran (70 mL) were added tetra butyl ammonium fluoride (14.64 mL, 1.0M, 14.66 mmol) and acetic acid (0.88 g, 14.66 mmol) at 0° C. The reaction mixture was allowed to stir at room temperature over a period of 1 h. The resulting reaction mixture was concentrated under reduced pressure and crude product obtained upon evaporation of the volatiles was purified through silica gel (230-400 mesh) column chromatography (14% ethyl acetate in hexane) to afford product 2-2 as a colorless liquid 3.0 g (64%). ¹H NMR (400 MHz, DMSO-d₆) δ 5.49 (d, 1H), 5.24-5.15 (m, 3H), 5.15-5.04 (m, 2H), 4.20 (quintet, 1H), 4.16-4.06 (m, 2H), 1.50-1.39 (m, 15H), 1.28 (d, 3H), 1.18 (t, 3H); MS m/z [M+NH₄]⁺ 496.7.

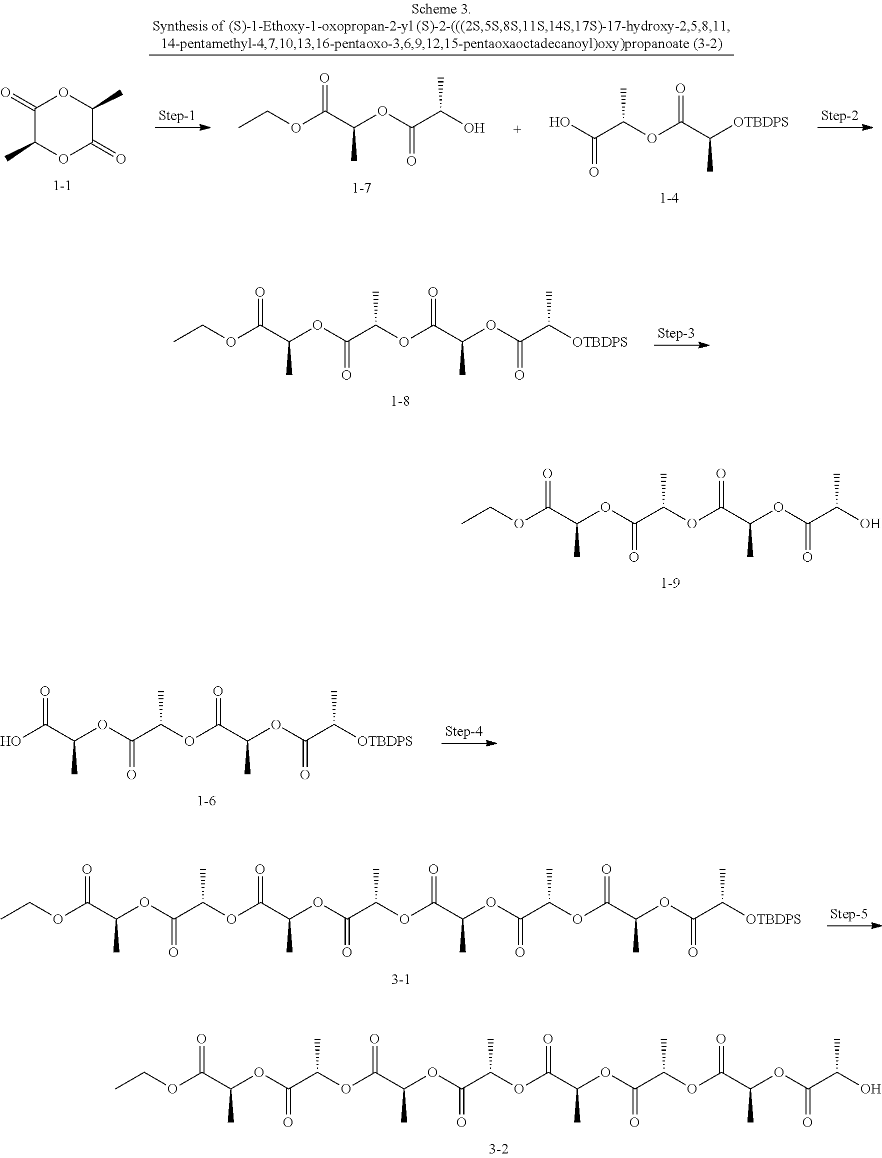

Step 1: Preparation of (S)-2-Hydroxy-propionic acid (S)-1-ethoxycarbonyl-ethyl ester (1-7):

To a solution of (3S,6S)-3,6-dimethyl-[1,4]-dioxane-2,5-dione 1-1 (5.0 g, 34.72 mmol) in toluene (100 mL) was added ethanol (1.92 mL, 31.98 mmol) and camphor sulfonic acid (0.8 g, 3.47 mmol) at 25-30° C. The reaction mixture was allowed to stir at 80° C. over a period of 2 h. The resulting reaction mixture was diluted with ethyl acetate (800 mL) and washed with water (2×200 mL). The crude product obtained upon evaporation of volatiles was purified through silica gel (230-400 mesh) column chromatography (13% ethyl acetate in hexane) to obtain product 1-7 as a colorless liquid 6.6 g (60%). ¹H-NMR (400 MHz, DMSO-d₆) δ 5.45 (d, 1H), 5.03 (q, 1H), 4.24-4.06 (m, 3H), 1.41 (d, J=7 Hz, 3H), 1.29 (d, J=7 Hz, 3H), 1.18 (t, 3H); MS m/z, [M+Na]⁺ 213.7.

Step 2: Preparation of (S)-2-(tert-Butyl-diphenyl-silanyloxy)-propionic acid (S)-1-[(S)-1-((S)-1-ethoxycarbonyl-ethoxycarbonyl)-ethoxycarbonyl]-ethyl ester (2-1): To a solution of (S)-2-(tert-Butyl-diphenyl-silanyloxy)-propionic acid (S)-1-carboxy-ethyl ester 1-4 (5.4 g, 13.68 mmol) in dichloromethane (60 mL) were added EDC.HCl (3.0 g, 15.78 mmol), (S)-2-Hydroxy-propionic acid (S)-1-ethoxycarbonyl-ethyl ester 1-7 (2.0 g, 10.52 mmol) and 4-dimethylaminopyridine (0.12 g, 1.05 mmol) at 0° C. The reaction mixture was allowed to stir at 25-30° C. over a period of 1 h. The resulting reaction mass was quenched with water (200 mL), extracted with dichloromethane (3×250 mL), dried over Na₂SO₄ and concentrated under reduced pressure. The crude product obtained upon evaporation of volatiles was purified through silica gel (230-400 mesh) column chromatography (3% ethyl acetate in hexane) to obtain product 2-1 as a colorless liquid 4.2 g (70%). ¹H-NMR (400 MHz, DMSO-d₆) δ 7.64-7.67 (m, 4H), 7.61-7.36 (m, 6H), 5.17 (q, 1H), 5.08 (q, 1H), 4.95 (q, 1H), 4.29 (q, 1H), 4.15-4.06 (m, 2H), 1.45 (d, J=7 Hz, 3H), 1.41 (d, J=7 Hz, 3H), 1.34-1.26 (m, 6H), 1.7 (t, 3H), 1.02 (s, 9H).

Step 3: Preparation of (S)-2-Hydroxy-propionic acid (S)-1-[(S)-1-((S)-1-ethoxycarbonyl-ethoxycarbonyl)-ethoxycarbonyl]-ethyl ester (1-9): To a solution of (S)-2-(tert-Butyl-diphenyl-silanyloxy)-propionic acid (S)-1-[(S)-1-((S)-1-ethoxycarbonyl-ethoxycarbonyl)-ethoxycarbonyl]-ethyl ester 1-8 (4 g, 6.99 mmol) in tetrahydrofuran (40 mL) were added tetra butyl ammonium fluoride (10.49 mL, 1.0M, 10.49 mmol) and acetic acid (0.63 g, 10.49 mmol) at 0° C. The reaction mixture was allowed to stir at room temperature over a period of 1 h. The resulting reaction mixture was concentrated under reduced pressure and crude product obtained upon evaporation of the volatiles was purified through silica gel (230-400 mesh) column chromatography (12% ethyl acetate in hexane) to give product 1-9 as a colorless liquid 1.0 g (43%). ¹H-NMR (400 MHz, DMSO-d₆) δ 5.50 (d, 1H), 5.21-5.03 (m, 3H), 4.23-4.05 (m, 3H), 1.51-1.38 (m, 9H), 1.28 (d, 3H), 1.71 (t, 3H).

Step 4: Preparation of (S)-2-(tert-Butyl-diphenyl-silanyloxy)-propionic acid (S)-1-{(S)-1-[(S)-1-((S)-1-{(S)-1-[(S)-1-((S)-1-ethoxycarbonyl-ethoxycarbonyl)-ethoxycarbonyl]-ethoxycarbonyl}-ethoxycarbonyl)-ethoxycarbonyl]-ethoxycarbonyl}-ethyl ester (3-1): To a solution of (S)-2-(tert-Butyl-diphenyl-silanyloxy)-propionic acid (S)-1-[(S)-1-((S)-1-carboxy-ethoxycarbonyl)-ethoxycarbonyl]-ethyl ester 1-6 (17.78 g, 32.69 mmol) in dichloromethane (84 mL) were added EDC.HCl (7.2 g, 37.72 mmol), (S)-2-Hydroxy-propionic acid (S)-1-[(S)-1-((S)-1-ethoxycarbonyl-ethoxycarbonyl)-ethoxycarbonyl]-ethyl ester 1-9 (8.4 g, 25.15 mmol) and 4-dimethylaminopyridine (0.30 g, 2.51 mmol) at 0° C. The reaction mixture was allowed to stir at 25-30° C. over a period of 1 h. The resulting reaction mass was quenched with water (500 mL), extracted with dichloromethane (4×250 mL), dried over Na₂SO₄ and concentrated under reduced pressure. The crude product obtained upon evaporation of volatiles was purified through silica gel (230-400 mesh) column chromatography (8% ethyl acetate in hexane) to obtain product 3-1 as a colorless liquid 10.0 g (47.6%). ¹H NMR (400 MHz, DMSO-d₆) δ 7.64-7.57 (m, 4H), 7.52-7.36 (m, 6H), 5.25-5.15 (m, 5H), 5.11 (q, 1H), 4.93 (q, 1H), 4.29 (q, 1H), 4.15-4.04 (m, 2H), 1.50-1.39 (m, 18H), 1.35-1.26 (m, 6H), 1.18 (t, 3H), 1.02 (s, 9H).

Step 5: Preparation of (S)-2-Hydroxy-propionic acid (S)-1-{(S)-1-[(S)-1-((S)-1-{(S)-1-[(S)-1-((S)-1-ethoxycarbonyl-ethoxycarbonyl)-ethoxycarbonyl]-ethoxycarbonyl}-ethoxycarbonyl)-ethoxycarbonyl]-ethoxycarbonyl}-ethyl ester (3-2): To a solution of (S)-2-(tert-Butyl-diphenyl-silanyloxy)-propionic acid (S)-1-{(S)-1-[(S)-1-((S)-1-{(S)-1-[(S)-1-((S)-1-ethoxycarbonyl-ethoxycarbonyl)-ethoxycarbonyl]-ethoxycarbonyl}-ethoxycarbonyl)-ethoxycarbonyl]-ethoxycarbonyl}-ethyl ester 3-1 (10.0 g, 11.63 mmol) in tetrahydrofuran (100 mL) were added tetra butyl ammonium fluoride (17.44 mL, 1.0M, 17.44 mmol) and acetic acid (0.88 g, 17.44 mmol) at 0° C. The reaction mixture was allowed to stir at room temperature over a period of 1 h. The resulting reaction mixture was concentrated under reduced pressure and crude product obtained upon evaporation of the volatiles was purified through silica gel (230-400 mesh) column chromatography (14% ethyl acetate in hexane) to give product 3-2 as a colorless liquid 4.5 g (62%). ¹H NMR (400 MHz, DMSO-d₆) δ 5.49 (d, 1H), 5.24-5.04 (m, 7H), 4.21 (quintet, 1H), 4.16-4.06 (m, 2H), 1.50-1.39 (m, 21H), 1.28 (d, 3H), 1.18 (t, 3H); MS m/z [M+NH₄]⁺ 640.8.

### Example 10. Synthesis of Loop Diuretics of Formula 1, Formula II, Formula III, or Formula IV

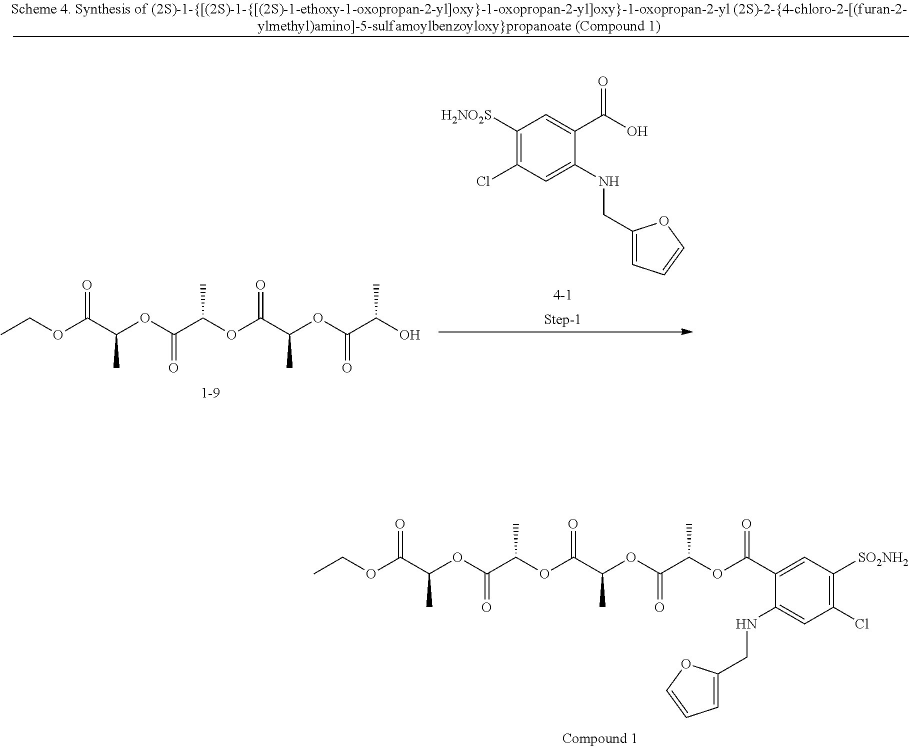 [SMILES: NS(=O)(=O)c1cc(C(=O)O)c(NCc2ccco2)cc1Cl ; CCOC(=O)[C@H](C)OC(=O)[C@H](C)OC(=O)[C@H](C)OC(=O)[C@H](C)O ; CCOC(=O)[C@H](C)OC(=O)[C@H](C)OC(=O)[C@H](C)OC(=O)[C@H](C)OC(=O)c1cc(S(N)(=O)=O)c(Cl)cc1NCc1ccco1]

To a solution of Furosemide (4-1, 100 mg, 0.30 mmol) in THF (5 mL) was added CDI (0.053 g, 0.33 mmol) at room temperature and stirred at 40° C. for 3 hours. (S)-2-Hydroxy-propionic acid (S)-1-[(S)-1-((S)-1-ethoxycarbonyl-ethoxycarbonyl)-ethoxycarbonyl]-ethyl ester (1-9, 0.15 g, 0.45 mmol) in THF (5 mL), followed by potassium tert-butoxide (0.05 g, 0.45 mmol) were being added to reaction mixture at room temperature and allowed to stir for 16 h at 40° C. The reaction mixture was diluted with ethyl acetate (100 mL) and washed in water (50 mL). The organic layer was dried over anhydrous sodium sulfate, filtered and concentrated to a pale yellow oil. The crude product obtained upon evaporation of volatiles was purified by silica gel (230-400 mesh) column chromatography (30% ethyl acetate in hexane) to give product Compound 1 as an off white solid 80 mg (41%). ¹H NMR (400 MHz, DMSO-d₆) δ 8.45 (s, 1H), 8.34 (t, 1H), 7.63-7.61 (m, 1H), 7.40 (s, 2H), 7.13 (s, 1H), 6.44-6.34 (m, 2H), 5.32 (q, 1H), 5.27-5.14 (m, 2H), 5.09 (q, 1H), 4.61 (d, 2H), 4.18-4.07 (m, 2H), 1.57 (d, 3H), 1.52-1.39 (m, 9H), 1.18 (t, 3H); MS m/z [M−H]⁻ 648.3.

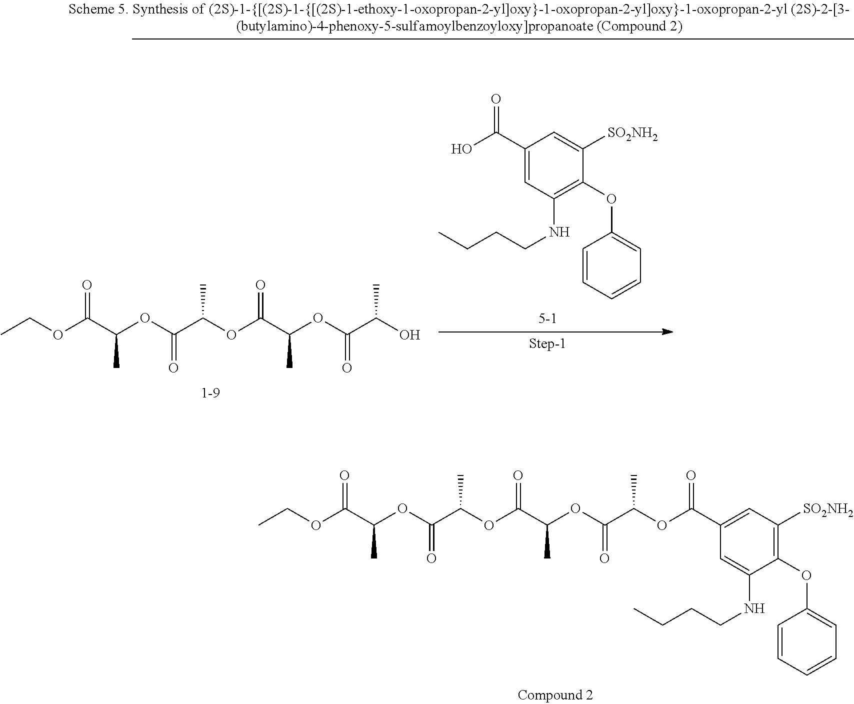 [SMILES: CCOC(=O)[C@H](C)OC(=O)[C@H](C)OC(=O)[C@H](C)OC(=O)[C@H](C)O ; CCCCNc1cc(C(=O)O)cc(S(N)(=O)=O)c1Oc1ccccc1 ; CCCCNc1cc(C(=O)O[C@@H](C)C(=O)O[C@@H](C)C(=O)O[C@@H](C)C(=O)O[C@@H](C)C(=O)OCC)cc(S(N)(=O)=O)c1Oc1ccccc1]

To a stirred solution of (S)-2-Hydroxy-propionic acid ( )-1-[(S)-1-((S)-1-ethoxycarbonyl-ethoxycarbonyl)-ethoxycarbonyl]-ethyl ester (1-9, 1.1 g, 3.29 mmol), in dichloromethane (25 mL) were added Bumetanide (5-1, 0.6 g, 1.64 mmol), N-hydroxybenzotriazole (HOBt) (0.11 g, 0.82 mmol), 4-dimethylaminopyridine (0.04 g, 0.32 mmol) at room temperature. The mixture was stirred for 5 min. and 1-ethyl-3-(3-dimethylaminopropyl) carbodiimide hydrochloride (EDC) (0.47 g, 2.46 mmol) was added and stirring was continued at 40° C. for a period of 16 h. The mixture was diluted with ethyl acetate (200 mL) and washed with water (100 mL). The organic layer was dried over anhydrous sodium sulfate, filtered, and concentrated to a sticky oil. The residue was purified by preparative HPLC and lyophilized to obtain pure product Compound 2 as an off white solid 150 mg (13%). ¹H NMR (400 MHz, DMSO-d₆) δ 7.71 (d, J=2 Hz 1H), 7.44-7.38 (m, 3H), 7.26 (t, 2H), 7.02 (t, 1H), 6.85 (dd, 2H), 5.36 (q, 1H), 5.30-5.16 (m, 3H), 5.10 (q, 1H), 4.17-4.06 (m, 2H), 3.10-3.02 (m, 2H), 1.60 (d, 3H), 1.56-1.32 (m, 11H), 1.19 (t, 3H), 1.14-1.04 (m, 2H), 0.77 (t, 3H); MS m/z [M+H]⁺ 682.0.

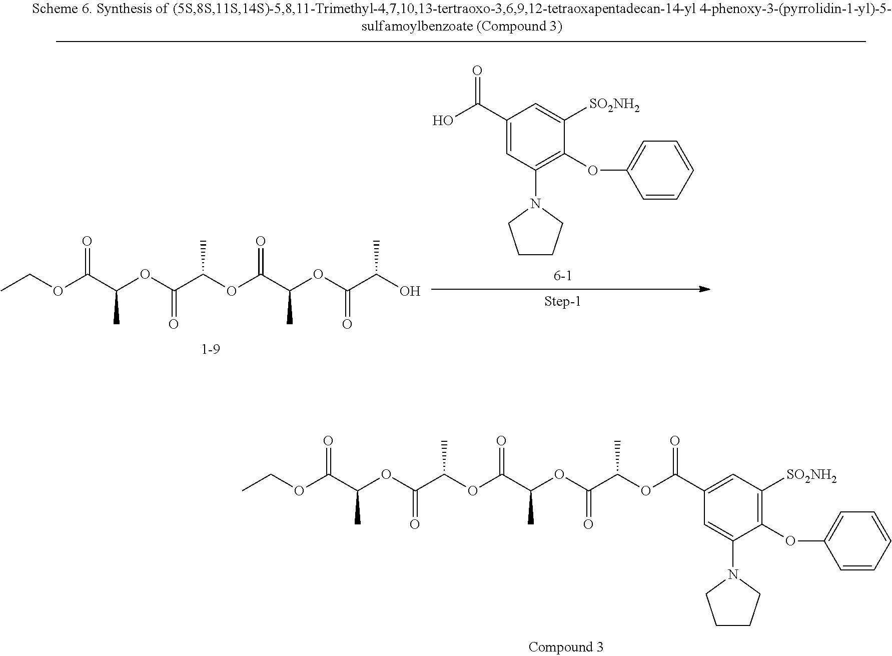 [SMILES: CCOC(=O)[C@H](C)OC(=O)[C@H](C)OC(=O)[C@H](C)OC(=O)[C@H](C)O ; NS(=O)(=O)c1cc(C(=O)O)cc(N2CCCC2)c1Oc1ccccc1 ; CCOC(=O)[C@H](C)OC(=O)[C@H](C)OC(=O)[C@H](C)OC(=O)[C@H](C)OC(=O)c1cc(N2CCCC2)c(Oc2ccccc2)c(S(N)(=O)=O)c1]

To a solution of Piretanide 6-1 in dichloromethane are added EDC.HCl, (S)-2-hydroxy-propionic acid (S)-1-[(S)-1-((S)-1-ethoxycarbonyl-ethoxycarbonyl)-ethoxycarbonyl]-ethyl ester 1-9, and 4-dimethyl amino pyridine at 0° C. The reaction mixture is allowed to stir at 25-30° C. over a period of approximately 2 h. The reaction mixture is diluted with water and extracted with dichloromethane. The combined organic layer is dried over sodium sulfate and concentrated under reduced pressure. The crude product obtained upon evaporation of volatiles is purified through silica gel column to obtain Compound 3.

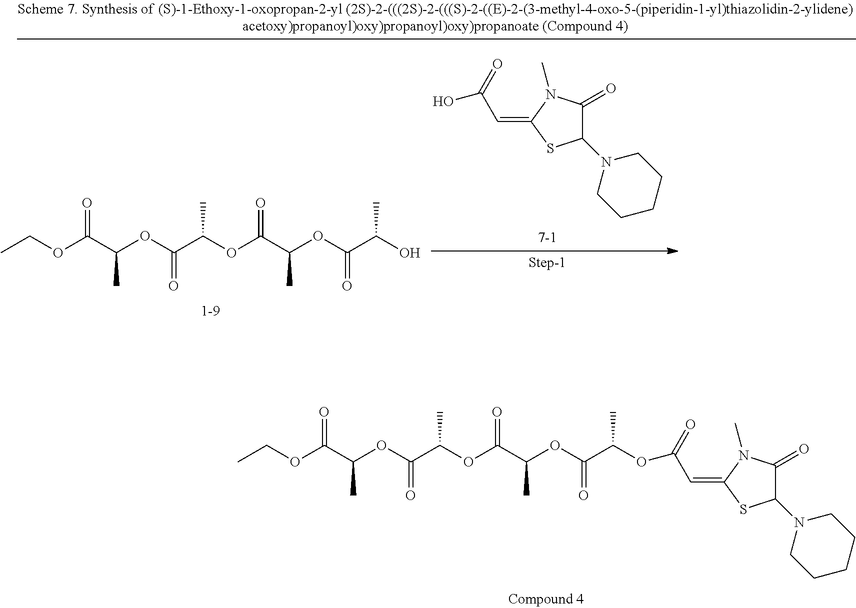 [SMILES: CCOC(=O)[C@H](C)OC(=O)[C@H](C)OC(=O)[C@H](C)OC(=O)[C@H](C)O ; CN1C(=O)C(N2CCCCC2)S/C1=C/C(=O)O ; CCOC(=O)[C@H](C)OC(=O)[C@H](C)OC(=O)[C@H](C)OC(=O)[C@H](C)OC(=O)/C=C1/SC(N2CCCCC2)C(=O)N1C]

To a solution of Ozolinone 7-1 in dichloromethane are added EDC.HCl, (S)-2-hydroxy-propionic acid (S)-1-[(S)-1-((S)-1-ethoxycarbonyl-ethoxycarbonyl)-ethoxycarbonyl]-ethyl ester 1-9, and 4-dimethyl amino pyridine at 0° C. The reaction mixture is allowed to stir at 25-30° C. over a period of approximately 2 h. The reaction mixture is diluted with water and extracted with dichloromethane. The combined organic layer is dried over sodium sulfate and concentrated under reduced pressure. The crude product obtained upon evaporation of volatiles is purified through silica gel column to obtain Compound 4.

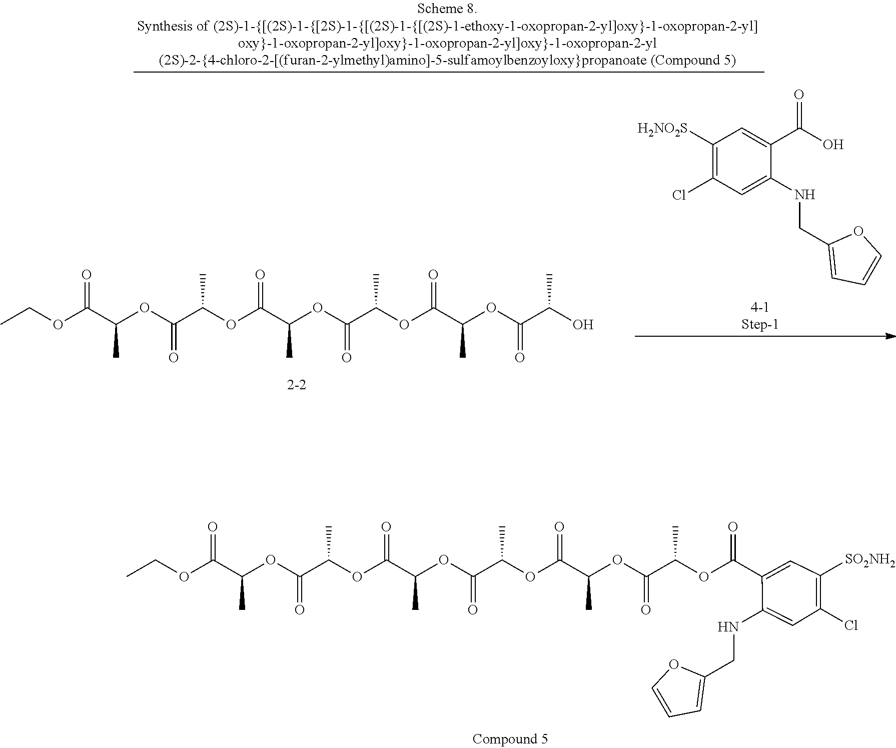 [SMILES: CCOC(=O)[C@H](C)OC(=O)[C@H](C)OC(=O)[C@H](C)OC(=O)[C@H](C)OC(=O)[C@H](C)OC(=O)[C@H](C)O ; NS(=O)(=O)c1cc(C(=O)O)c(NCc2ccco2)cc1Cl ; CCOC(=O)[C@H](C)OC(=O)[C@H](C)OC(=O)[C@H](C)OC(=O)[C@H](C)OC(=O)[C@H](C)OC(=O)[C@H](C)OC(=O)c1cc(S(N)(=O)=O)c(Cl)cc1NCc1ccco1]

To a stirred solution of (S)-2-Hydroxy-propionic acid (S)-1-((S)-1-{(S)-1-[(S)-1-((S)-1-ethoxycarbonyl-ethoxycarbonyl)-ethoxycarbonyl]-ethoxycarbonyl}-ethoxycarbonyl)-ethyl ester (2-2, 2.1 g, 4.3 mmol) in dichloromethane (25 mL) were added Furosemide (4-1, 0.73 g, 2.19 mmol), N-hydroxybenzotriazole (HOBt) (0.15 g, 1.11 mmol) and 4-dimethylaminopyridine (0.05 g, 0.44 mmol). The mixture was stirred at room temperature for 5 min, and, 1-ethyl-3-(3-dimethylaminopropyl) carbodiimide hydrochloride (EDC) (0.64 g, 3.3 mmol) was added and stirring was continued at 40° C. fort a period of 16 h. The mixture was diluted with ethyl acetate (200 mL) and washed with water (100 mL). The organic layer was dried over anhydrous sodium sulfate, filtered, and concentrated to a sticky oil. The residue was purified by preparative HPLC and lyophilized to obtain pure product Compound 5 as an off white solid 260 mg (14%). ¹H NMR (400 MHz, DMSO-d₆) δ 8.45 (s, 1H), 8.33 (t, 1H), 7.63-7.61 (m, 1H), 7.40 (s, 2H), 7.13 (s, 1H), 6.43-6.33 (m, 2H), 5.32 (q, 1H), 5.27-5.15 (m, 4H), 5.09 (q, 1H), 4.60 (d, 2H), 4.17-4.04 (m, 2H), 1.57 (d, 3H), 1.51-1.37 (m, 15H), 1.18 (t, 3H); MS m/z [M−H]⁻ 790.2.

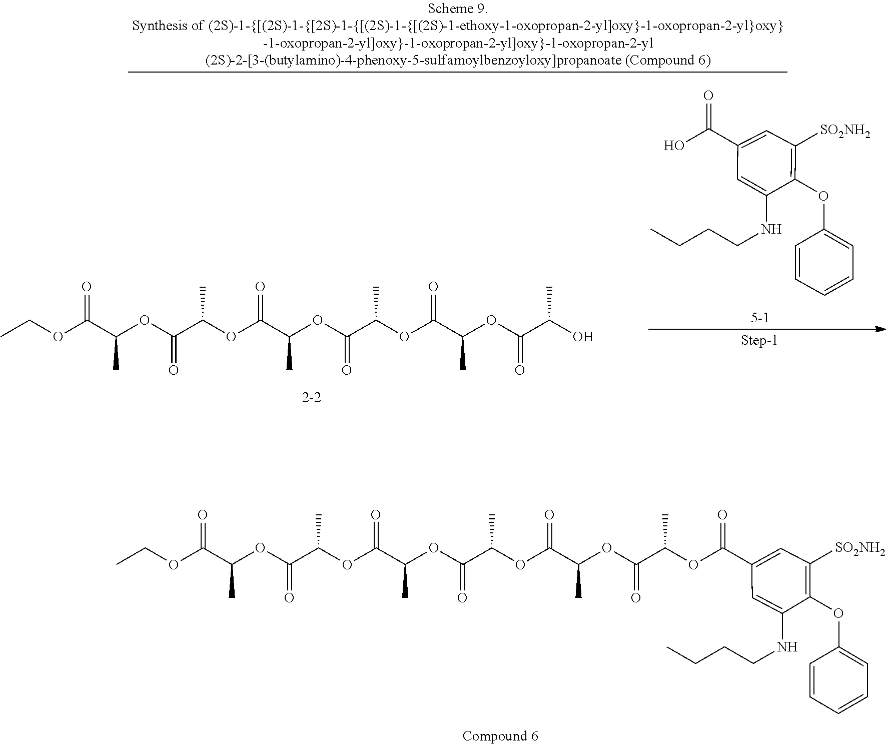 [SMILES: CCOC(=O)[C@H](C)OC(=O)[C@H](C)OC(=O)[C@H](C)OC(=O)[C@H](C)OC(=O)[C@H](C)OC(=O)[C@H](C)O ; CCCCNc1cc(C(=O)O)cc(S(N)(=O)=O)c1Oc1ccccc1 ; CCCCNc1cc(C(=O)O[C@@H](C)C(=O)O[C@@H](C)C(=O)O[C@@H](C)C(=O)O[C@@H](C)C(=O)O[C@@H](C)C(=O)O[C@@H](C)C(=O)OCC)cc(S(N)(=O)=O)c1Oc1ccccc1]

To a stirred solution of (S)-2-Hydroxy-propionic acid (S)-1-((S)-1-{(S)-1-[(S)-1-((S)-1-ethoxycarbonyl-ethoxycarbonyl)-ethoxycarbonyl]-ethoxycarbonyl}-ethoxycarbonyl)-ethyl ester (2-2, 1.57 g, 3.29 mmol) in dichloromethane (25 mL), were added Bumetanide (5-1, 0.6 g, 1.64 mmol), N-hydroxybenzotriazole (HOBt) (0.11 g, 0.82 mmol), 4-dimethylaminopyridine (0.04 g, 0.32 mmol). Stirring was continued at room temperature for 5 min., before 1-ethyl-3-(3-dimethylaminopropyl) carbodiimide hydrochloride (EDC) (0.47 g, 2.4 mmol) was added and stirring was continued at 40° C. for a period of 16 h. The reaction was diluted with ethyl acetate (200 mL) and washed with water (100 mL). The organic layer was dried over anhydrous sodium sulfate, filtered, and concentrated to a sticky oil. The residue was purified by preparative HPLC and lyophilized to obtain pure product Compound 6 as an off white solid 240 mg (17%). ¹H NMR (400 MHz, DMSO-d₆) δ 7.71 (d, J=2 Hz 1H), 7.43-7.37 (m, 3H), 7.27 (t, 2H), 7.01 (t, 1H), 6.85 (dd, 2H), 5.37 (q, 1H), 5.29-5.15 (m, 5H), 5.09 (q, 1H), 4.17-4.06 (m, 2H), 3.10-3.02 (m, 2H), 1.60 (d, 3H), 1.54-1.31 (m, 17H), 1.18 (t, 3H), 1.14-1.04 (m, 2H), 0.77 (t, 3H); MS m/z [M+H]-826.1.

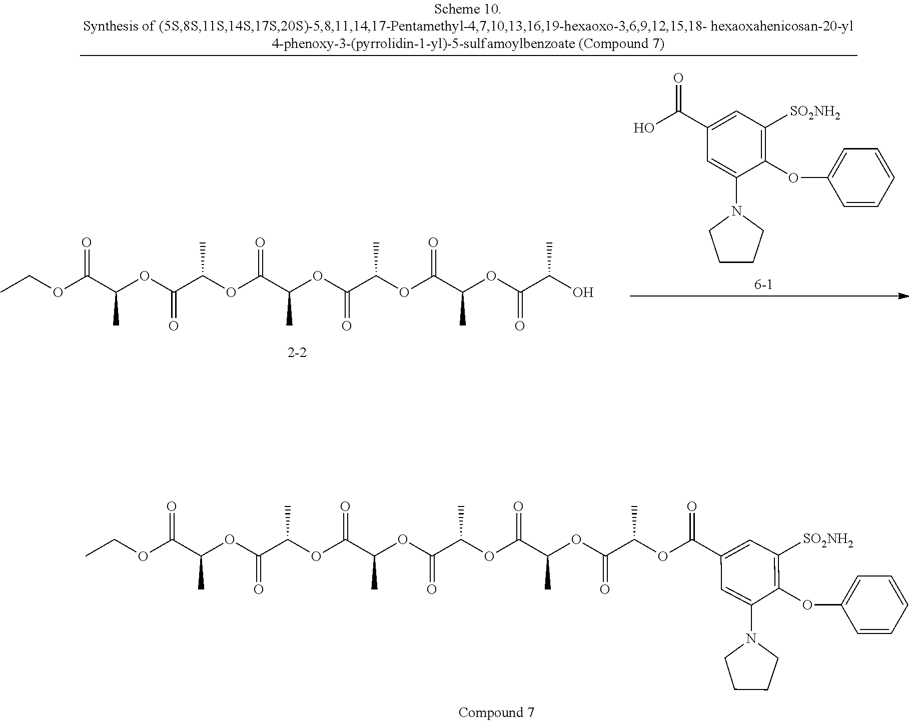 [SMILES: CCOC(=O)[C@H](C)OC(=O)[C@H](C)OC(=O)[C@H](C)OC(=O)[C@H](C)OC(=O)[C@H](C)OC(=O)[C@H](C)O ; NS(=O)(=O)c1cc(C(=O)O)cc(N2CCCC2)c1Oc1ccccc1 ; CCOC(=O)[C@H](C)OC(=O)[C@H](C)OC(=O)[C@H](C)OC(=O)[C@H](C)OC(=O)[C@H](C)OC(=O)[C@H](C)OC(=O)c1cc(N2CCCC2)c(Oc2ccccc2)c(S(N)(=O)=O)c1]

To a solution of Piretanide 6-1 in dichloromethane are added EDC.HCl, (S)-2-hydroxy-propionic acid (S)-1-[(S)-1-((S)-1-ethoxycarbonyl-ethoxycarbonyl)-ethoxycarbonyl]-ethyl ester 1-9, and 4-dimethyl amino pyridine at 0° C. The reaction mixture is allowed to stir at 25-30° C. over a period of approximately 2 h. The reaction mixture is diluted with water and extracted with dichloromethane. The combined organic layer is dried over sodium sulfate and concentrated under reduced pressure. The crude product obtained upon evaporation of volatiles is purified through silica gel column to obtain Compound 7.

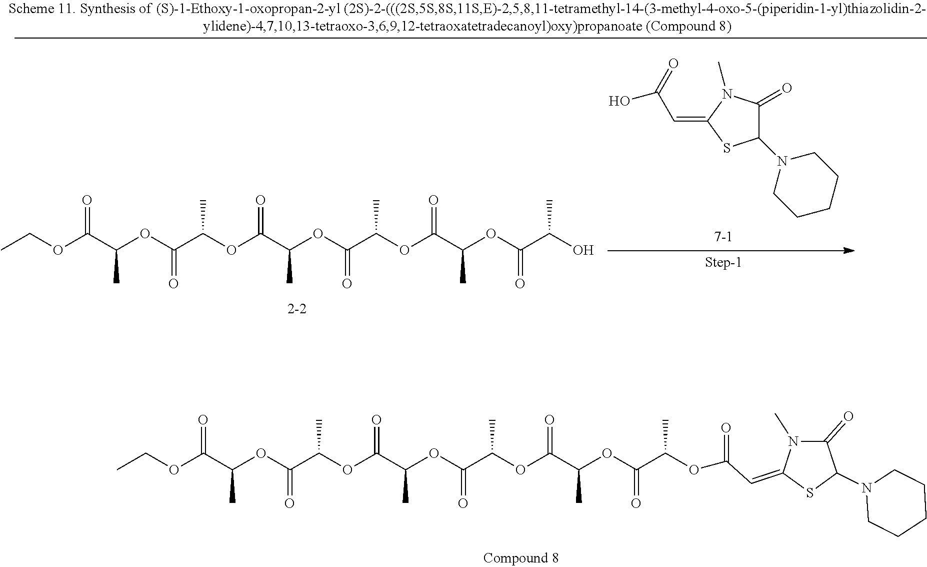 [SMILES: CCOC(=O)[C@H](C)OC(=O)[C@H](C)OC(=O)[C@H](C)OC(=O)[C@H](C)OC(=O)[C@H](C)OC(=O)[C@H](C)O ; CN1C(=O)C(N2CCCCC2)S/C1=C/C(=O)O ; CCOC(=O)[C@H](C)OC(=O)[C@H](C)OC(=O)[C@H](C)OC(=O)[C@H](C)OC(=O)[C@H](C)OC(=O)[C@H](C)OC(=O)/C=C1/SC(N2CCCCC2)C(=O)N1C]

To a solution of Ozolinone 7-1 in dichloromethane are added EDC.HCl, (S)-2-hydroxy-propionic acid (S)-1-[(S)-1-((S)-1-ethoxycarbonyl-ethoxycarbonyl)-ethoxycarbonyl]-ethyl ester 1-9, and 4-dimethyl amino pyridine at 0° C. The reaction mixture is allowed to stir at 25-30° C. over a period of approximately 2 h. The reaction mixture is diluted with water and extracted with dichloromethane. The combined organic layer is dried over sodium sulfate and concentrated under reduced pressure. The crude product obtained upon evaporation of volatiles is purified through silica gel column to obtain Compound 8.

### Example 11. Synthesis of Timolol-Bumetanide Glycolamide Bis-Prodrugs and Bumetanide Acyl Acetal Prodrugs

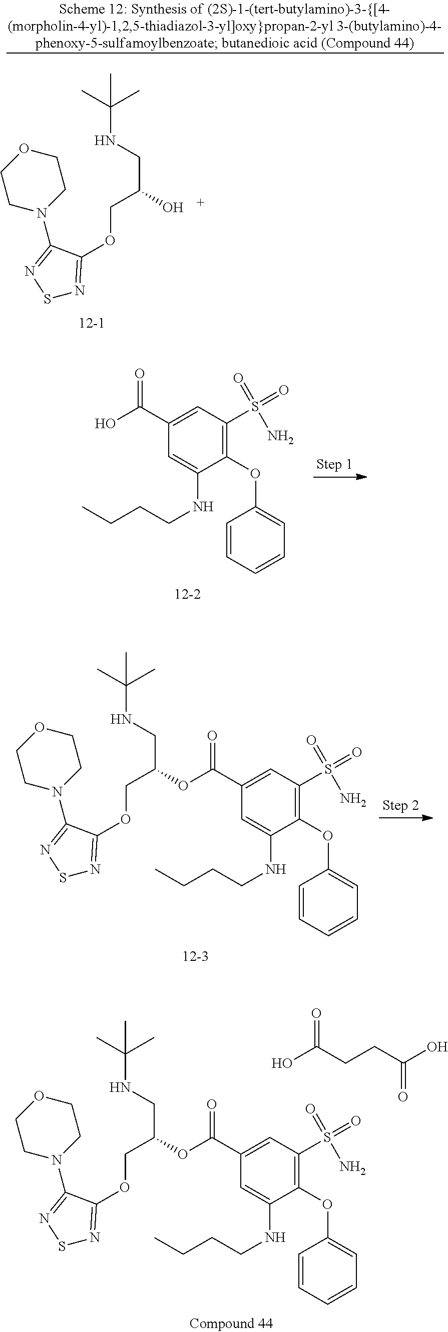 [SMILES: CC(C)(C)NC[C@H](O)COc1nsnc1N1CCOCC1 ; CCCCNc1cc(C(=O)O)cc(S(N)(=O)=O)c1Oc1ccccc1 ; CCCCNc1cc(C(=O)O[C@@H](CNC(C)(C)C)COc2nsnc2N2CCOCC2)cc(S(N)(=O)=O)c1Oc1ccccc1 ; CCCCNc1cc(C(=O)O[C@@H](CNC(C)(C)C)COc2nsnc2N2CCOCC2)cc(S(N)(=O)=O)c1Oc1ccccc1 ; O=C(O)CCC(=O)O]

Step 1: Preparation of (2S)-1-(tert-butylamino)-3-{[4-(morpholin-4-yl)-1,2,5-thiadiazol-3-yl]oxy}propan-2-yl 3-(butylamino)-4-phenoxy-5-sulfamoylbenzoate (12-3): To a solution of timolol 12-1 (4.5 g, 14.2 mmol) and bumetanide 12-2 (5.7 g, 15.6 mmol) in dichloromethane (50 mL) were added EDC.HCl (4.07 g, 21.3 mmol) and 4-Dimethylaminopyridine (0.17 g, 1.58 mmol) at 0-5° C. The reaction mixture was allowed to stir at 25-30° C. for 1 h. The resulting reaction mixture was diluted with ethyl acetate (500 mL) and washed with water (2×150 mL), the organic layer was separated and dried over sodium sulfate and concentrated under reduced pressure at 45° C. The crude compound was purified by silica gel (230-400 mesh) column chromatography to obtain product 12-3 as an off white solid 2.8 g (29%).

Step 2: Preparation of (2S)-1-(tert-butylamino)-3-{[4-(morpholin-4-yl)-1,2,5-thiadiazol-3-yl]oxy}propan-2-yl 3-(butylamino)-4-phenoxy-5-sulfamoylbenzoate; butanedioic acid (Compound 44): To solution of (2S)-1-(tert-butylamino)-3-{[4-(morpholin-4-yl)-1,2,5-thiadiazol-3-yl]oxy}propan-2-yl 3-(butylamino)-4-phenoxy-5-sulfamoylbenzoate 12-3 (1.3 g, 1.96 mmol) in acetone (30 mL) was added succinic acid (0.20 g, 1.7 mmol) and allowed to stir for 5 min at 0-5° C. The resulting reaction mixture was concentrated under reduced pressure at 45° C. to obtain product Compound 44 as an off white solid 1.3 g (86%). ¹H-NMR (400 MHz, DMSO-d6) δ 7.69 (d, J=2 Hz 1H), 7.41-7.35 (m, 3H), 7.26 (t, J=8 Hz, 2H), 7.01 (t, J=8 Hz, 1H), 6.83 (d, J=8 Hz, 2H), 5.52-5.42 (m, 1H), 5.24 (t, 1H), 4.83-4.75 (m, 1H), 4.62-4.53 (m, 1H), 3.61-3.50 (m, 4H), 3.4-3.2 (m, 4H), 3.1-2.9 (m, 4H), 2.38 (s, 4H, Succinate), 1.35 (quintet, 2H), 1.14-1.01 (m, 11H), 0.75 (t, 3H). MS m/z [M+H]⁺ 664.0.

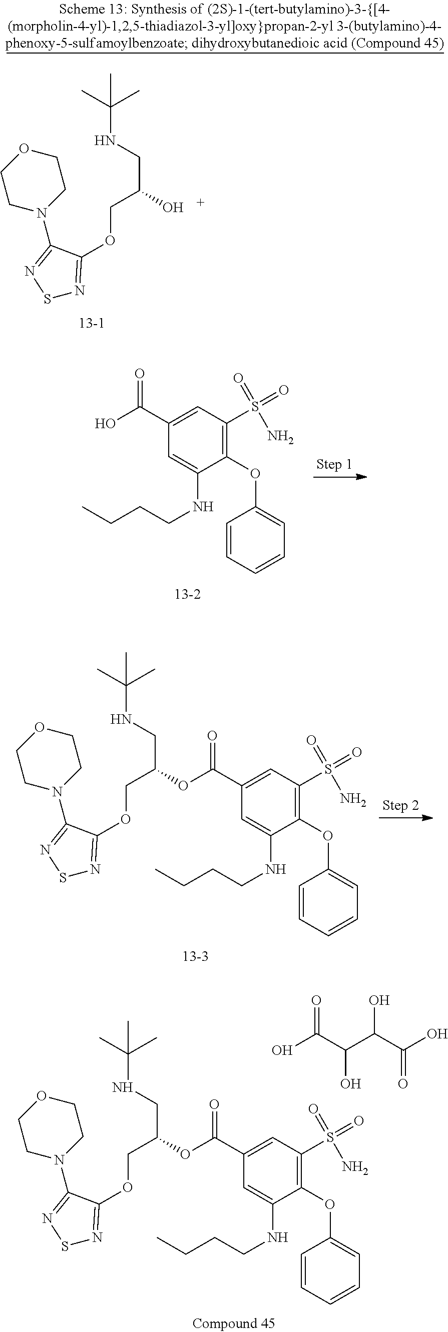

Step 1: Preparation of (2S)-1-(tert-butylamino)-3-{[4-(morpholin-4-yl)-1,2,5-thiadiazol-3-yl]oxy}propan-2-yl 3-(butylamino)-4-phenoxy-5-sulfamoylbenzoate (13-3): To a solution of timolol 13-1 (4.5 g, 14.2 mmol) and bumetanide 13-2 (5.7 g, 15.6 mmol) in dichloromethane (50 mL) were added EDC.HCl (4.07 g, 21.3 mmol) and 4-Dimethylaminopyridine (0.17 g, 1.58 mmol) at 0-5° C. The reaction mixture was allowed to stir at 25-30° C. for 1 h. The resulting reaction mixture was diluted with ethyl acetate (200 mL) and washed with water (2×200 mL). The organic layer was dried over sodium sulfate and concentrated under reduced pressure at 45° C. The crude compound was purified by silica gel (230-400 mesh) column chromatography to obtain product 13-3 as an off white solid 2.8 g (29%).

Step 2: Preparation of (2S)-1-(tert-butylamino)-3-{[4-(morpholin-4-yl)-1,2,5-thiadiazol-3-yl]oxy}propan-2-yl 3-(butylamino)-4-phenoxy-5-sulfamoylbenzoate; 2,3-dihydroxybutanedioic acid (Compound 45): To a solution of (2S)-1-(tert-butylamino)-3-{[4-(morpholin-4-yl)-1,2,5-thiadiazol-3-yl]oxy}propan-2-yl 3-(butylamino)-4-phenoxy-5-sulfamoylbenzoate 13-3 (1.5 g, 2.2 mmol) in acetone (15 mL) was added L-tartaric acid (0.305 g, 2.0 mmol) and stirred for 5 min at 0-5° C. The resulting reaction mixture was concentrated under reduced pressure at 45° C. to obtain product Compound 45 as an off white solid 1.2 g (66%). ¹H-NMR (400 MHz, DMSO-d6) δ 7.70 (d, J=2 Hz 1H), 7.43-7.36 (m, 3H), 7.26 (t, J=8 Hz, 2H), 7.02 (t, J=8 Hz, 1H), 6.84 (d, J=8 Hz, 2H), 5.62-5.52 (m, 1H), 5.26 (t, 1H), 4.84-4.76 (m, 1H), 4.64-4.55 (m, 1H), 4.04 (s, 2H, Tartrate), 3.61-3.52 (m, 4H), 3.4-3.1 (m, 6H), 3.04 (q, 2H), 1.35 (quintet, 2H), 1.18 (s, 9H), 1.15-1.00 (m, 2H), 0.75 (t, 3H). MS m/z [M+H]⁺ 664.0.

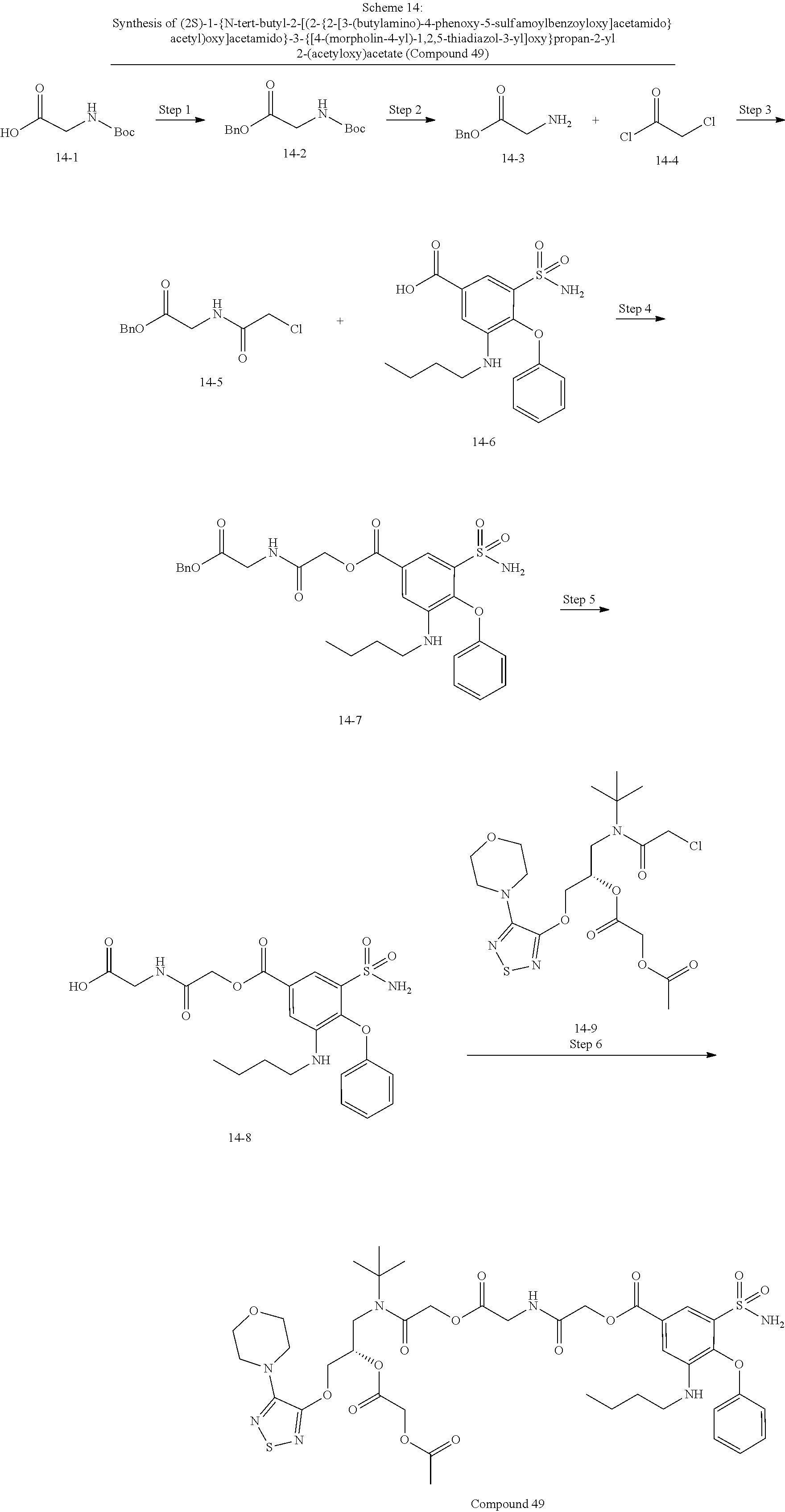

Step 1: Preparation of benzyl [(tert-butoxycarbonyl)amino]acetate (14-2): To a solution of [(tert-butoxycarbonyl) amino]acetic acid 14-1 (35 g, 199.78 mmol) in dichloromethane (50 mL) were added EDC.HCl (57.24 g, 299.6 mmol), benzyl alcohol (17.28 g, 159.82 mmol) and 4-Dimethylaminopyridine (2.43 g, 19.97 mmol) at 0-5° C. The reaction mixture was allowed to stir at 25-30° C. for 1 h. The resulting reaction mixture was diluted with ethyl acetate (800 mL) and washed with water (500 mL). The organic layer was dried over sodium sulfate and concentrated under reduced pressure at 45° C. The crude compound was purified by silica gel (60-120 mesh) column chromatography to obtain product 14-2 as a colourless wax 52.0 g (98%).

Step 2: Preparation of benzyl aminoacetate (14-3): To a solution of benzyl [(tert-butoxycarbonyl)amino]acetate 14-2 (52.0 g, 196 mmol) in dichloromethane (520 mL) was added TFA (208 mL) at 0-5° C. The reaction mixture was allowed to stir at 25-30° C. for 1 h. The resulting reaction mixture was concentrated under reduced pressure at 45° C. to obtain product 14-3 as a brown wax 70.0 g (Crude).

Step 3: Preparation of benzyl (2-chloroacetamido)acetate (14-5): To a solution of benzyl aminoacetate 14-3 (70.0 g, 423.8 mmol) in dichloromethane (700 mL) were added triethylamine (173.8 mL, 1271 mmol), 4-Dimethylaminopyridine (5.17 g, 43.38 mmol) and chloroacetyl chloride 14-4 (33.69 mL, 423.8 mmol) drop-wise at 0-5° C. The reaction mixture was allowed to stir at 25-30° C. for 1 h. The resulting reaction mixture was diluted with ethyl acetate (1.2 L) and washed with water (2×500 mL). The organic layer was dried over sodium sulfate and concentrated under reduced pressure at 45° C. The crude was purified by silica gel (230-400 mesh) column chromatography to obtain product 14-5 as a colourless wax 19.35 g (18.8%).

Step 4: Preparation of benzyl 2-{2-[3-(butylamino)-4-phenoxy-5-sulfamoylbenzoyloxy]acetamido}acetate (14-7): To a solution of bumetanide 14-6 (30.0 g, 82.32 mmol) in N, N-Dimethylformamide (150 mL) were added triethylamine (28.14 mL, 20.58 mmol), NaI (14.8 g, 98.78 mmol) and benzyl (2-chloroacetamido)acetate 14-5 (23.87 g, 98.78 mmol) at 0-5° C. The reaction mixture was allowed to stir at 25-30° C. for 2 h. The resulting reaction mixture was diluted with ethyl acetate (750 mL) and washed with water (2×250 mL). The organic layer was dried over sodium sulfate and concentrated under reduced pressure at 45° C. The crude was purified by silica gel (230-400 mesh) column chromatography to obtain product 14-7 as an off white solid 19.2 g (40.59%).

Step 5: Preparation of 2-{2-[3-(butylamino)-4-phenoxy-5-sulfamoylbenzoyloxy]acetamido}acetic acid (14-8): 10% Pd/C (2 g, 50% wet, 20% w/w) was added to a solution of 2-{2-[3-(butylamino)-4-phenoxy-5-sulfamoylbenzoyloxy]acetamido}acetate 14-7 (10 g, 17.55 mmol) in methanol (70 mL) and dichloromethane (30 mL) taken in Parr-shaker vessel. The reaction mixture was hydrogenated with 5 kg/cm²hydrogen pressure at 25-30° C. for 1 h. The resulting reaction mixture was filtered through celite bed. The filtrate was concentrated under reduced pressure at 45° C. to obtain product 14-8 as an off white solid 6.0 g (71%).

Step 6: Preparation of (2S)-1-{N-tert-butyl-2-[(2-{2-[3-(butylamino)-4-phenoxy-5-sulfamoyl benzoyloxy]acetamido}acetyl)oxy]acetamido}-3-{[4-(morpholin-4-yl)-1,2,5-thiadiazol-3-yl]oxy}propan-2-yl 2-(acetyloxy)acetate (Compound 49): To a solution of 2-{2-[3-(butylamino)-4-phenoxy-5-sulfamoylbenzoyloxy]acetamido}acetic acid 14-8 (2.1 g, 4.38 mmol) in N, N-Dimethylformamide (10 V) were added triethylamine (1.49 mL, 10.95 mmol), NaI (0.788 g, 5.26 mmol) and (2S)-1-(N-tert-butyl-2-chloroacetamido)-3-{[4-(morpholin-4-yl)-1,2,5-thiadiazol-3-yl]oxy}propan-2-yl-2-(acetyloxy) acetate 14-9 (2.59 g, 5.26 mmol) at 0-5° C. The reaction mixture was allowed to stir at 55° C. for 5 h. The resulting reaction mixture was diluted with ethyl acetate (15 V) and washed with water (2×10 V). The organic layer was dried over sodium sulfate and concentrated under reduced pressure at 45° C. The crude compound was purified by silica gel (230-400 mesh) column chromatography to obtain product Compound 49 as a white solid 1.4 g (34.2%). ¹H-NMR (400 MHz, DMSO-d6) δ 8.71 (t, 1H), 7.75 (d, J=2 Hz, 1H), 7.46 (d, J=2 Hz, 1H), 7.39 (s, 2H), 7.27 (t, J=8 Hz, 2H), 7.02 (t, J=8 Hz, 1H), 6.85 (d, J=8 Hz, 2H), 5.51-5.40 (m, 1H), 5.15 (t, 1H), 4.96 (d, 1H), 4.87-4.65 (m, 5H), 4.63-4.56 (m, 1H), 4.50-4.41 (m, 1H), 4.09-3.94 (m, 2H), 3.71-3.56 (m, 6H), 3.43-3.3 (m, 4H), 3.07 (q, 2H), 2.09 (s, 3H), 1.42-1.23 (m, 11H), 1.15-1.02 (m, 2H), 0.76 (t, 3H). MS m/z [M+H]⁺ 937.2.

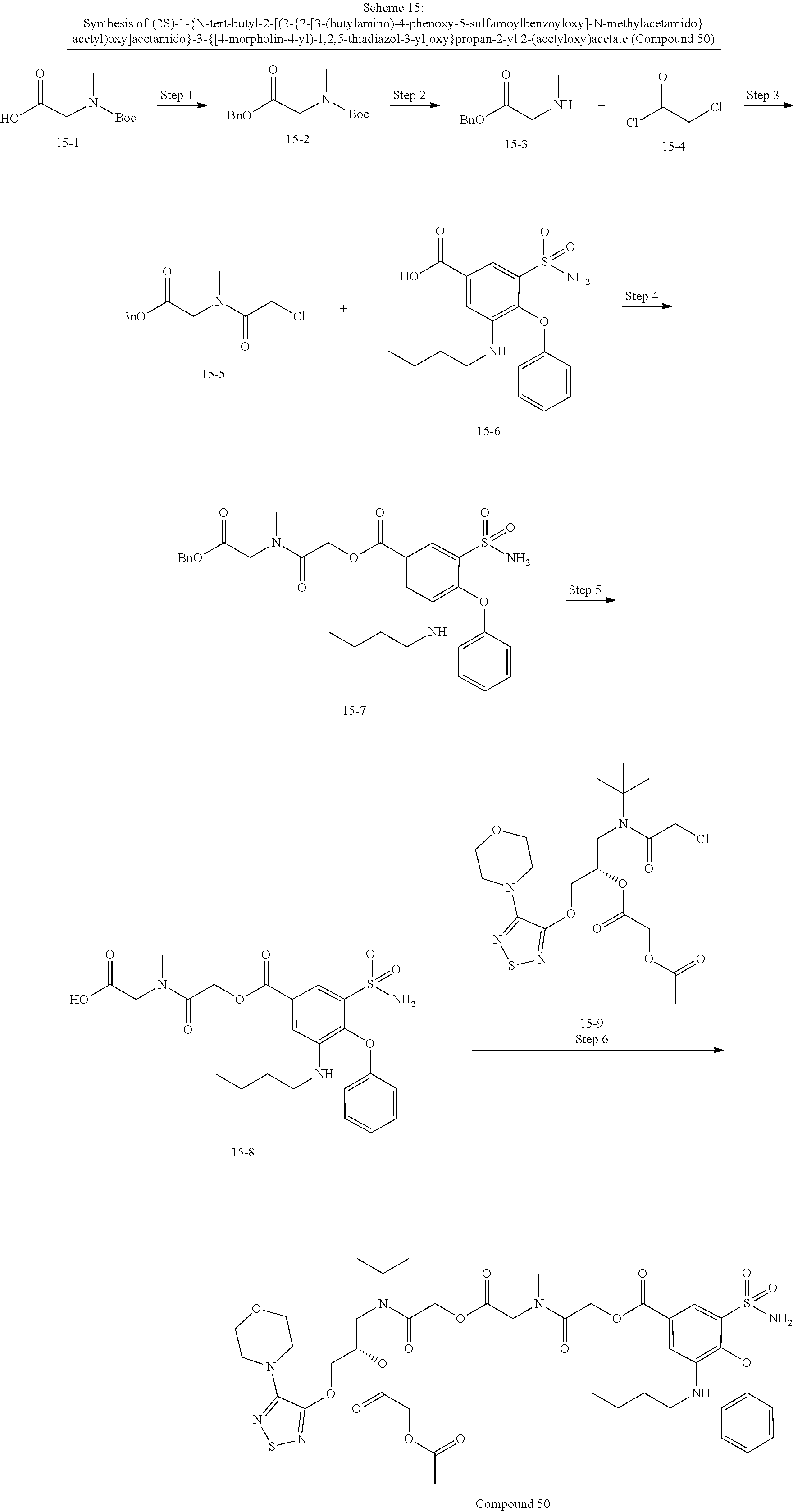

Step 1: Preparation of benzyl [(tert-butoxycarbonyl)(methyl)amino]acetate (15-2): To a solution of [(tert-butoxycarbonyl)(methyl)amino]acetic acid 15-1 (50.0 g, 264.0 mmol) in dichloromethane (500 mL) were added EDC.HCl (75.71 g, 396.0 mmol), benzyl alcohol (22.86 g, 211.0 mmol) and 4-Dimethylaminopyridine (3.22 g, 26.0 mol) at 0-5° C. The reaction mixture was allowed to stir at 25-30° C. for 2 h. The resulting reaction mixture was diluted with ethyl acetate (800 mL) and washed with water (300 mL). The organic layer was dries over sodium sulfate and concentrated under reduced pressure at 45° C. The crude compound was purified by silica gel (60-120 mesh) column chromatography to obtain product 15-2 as a colourless wax 54.0 g (73%).

Step 2: Preparation of benzyl (methylamino)acetate (15-3): To a solution of benzyl [(tert-butoxycarbonyl)(methyl)amino]acetate 15-2 (54.0 g, 193.0 mmol) in dichloromethane (540 mL) was added TFA (216 mL) at 0-5° C. The reaction mixture was allowed to stir at 25-30° C. for 1 h. The resulting reaction mixture was concentrated under reduced pressure at 45° C. to obtain product 15-3 as a brown wax 85.0 g (crude compound obtained as a TFA salt was taken as such into next step).

Step 3: Preparation of benzyl [(chloroacetyl)(methyl)amino]acetate (15-5): To a solution of benzyl (methylamino)acetate 15-3 (85.0 g, 474.0 mmol) in dichloromethane (850 mL) were added triethylamine (194.57 mL, 1422 mmol), 4-Dimethylaminopyridine (5.78 g, 47.0 mmol) and chloro acetyl chloride 15-4 (56.56 mL, 711.0 mmol) slowly at 0-5° C. The reaction mixture was allowed to stir at 25-30° C. for 1 h. The resulting reaction mixture was diluted with ethyl acetate (1.2 L) and washed with water (2×500 mL). The organic layer was dried over sodium sulfate and concentrated under reduced pressure at 45° C. The crude compound was purified by silica gel (230-400 mesh) column chromatography to obtain product 15-5 as a colourless wax 23.0 g (18.9

Step 4: Preparation of benzyl 2-{2-[3-(butylamino)-4-phenoxy-5-sulfamoylbenzoyloxy]-N-methylacetamido}acetate (15-7): To a solution of bumetanide 15-6 (19 g, 52.13 mmol) in N,N-Dimethylformamide (100 mL) were added K₂CO₃ (8.64 g, 62.55 mmol), TBAI (1.92 g, 5.21 mmol) and benzyl [(chloroacetyl)(methyl)amino]acetate 15-5 (17.33 g, 67.78 mmol) at 0-5° C. The reaction mixture was allowed to stir at 25-30° C. for 4 h. The resulting reaction mixture was diluted with ethyl acetate (400 mL) and washed with water (2×250 mL). The organic layer was dried over sodium sulfate and concentrated under reduced pressure at 45° C. The crude compound was purified by silica gel (230-400 mesh) column chromatography to obtain product 15-7 as an off white solid 21.5 g (69%).

Step 5: Preparation of 2-{2-[3-(butylamino)-4-phenoxy-5-sulfamoylbenzoyloxy]-N-methylacetamido}acetic acid (15-8): 10% Pd/C (4 g, 50% wet, 20% w/w) was added to a solution of benzyl 2-{2-[3-(butylamino)-4-phenoxy-5-sulfamoylbenzoyloxy]-N-methylacetamido} acetate 15-7 (21.5 g, 36.83 mmol) in methanol (150 mL) and dichloromethane (45 mL) taken in a Parr-shaker vessel. The reaction mixture was hydrogenated with 5 kg/cm² hydrogen pressure at 25-30° C. for 1 h. The resulting reaction mixture was filtered through celite bed. The filtrate was concentrated under reduced pressure at 45° C. to obtain 15-8 as an off white solid 15.5 g (85.3%).

Step 6: Preparation of (2S)-1-{N-tert-butyl-2-[(2-{2-[3-(butylamino)-4-phenoxy-5-sulfamoylbenzoyloxy]-N-methylacetamido}acetyl)oxy]acetamido}-3-{[4-(morpholin-4-yl)-1,2,5-thiadiazol-3-yl]oxy}propan-2-yl 2-(acetyloxy)acetate (Compound 50): To a solution of 2-{2-[3-(butylamino)-4-phenoxy-5-sulfamoylbenzoyloxy]-N-methylacetamido}acetic acid 15-8 (2.1 g, 4.25 mmol) in N, N-Dimethylformamide (10 mL) were added triethylamine (1.16 mL, 8.5 mmol), NaI (0.76 g, 5.1 mmol) and (2S)-1-(N-tert-butyl-2-chloroacetamido)-3-{[4-(morpholin-4-yl)-1,2,5-thiadiazol-3-yl]oxy}propan-2-yl2-(acetyloxy) acetate 15-9 (2.52 g, 5.11 mmol) at 0-5° C. The reaction mixture was allowed to stir at 55° C. for 2 h. The resulting reaction mixture was diluted with ethyl acetate (200 mL) and washed with water (2×50 mL). The organic layer was dried over sodium sulfate and concentrated under reduced pressure at 45° C. The crude compound was purified by silica gel (230-400 mesh) column chromatography to obtain product Compound 50 as an off white solid 1.6 g (39.6%). 122-s6, ¹H-NMR (400 MHz, DMSO-d6) δ 7.77-7.71 (m, 1H), 7.47-7.36 (m, 3H), 7.27 (dd, 2H), 7.01 (t, 1H), 6.87-6.81 (m, 2H), 5.52-5.41 (m, 1H), 5.20-4.55 (m, 8H), 4.51-4.22 (m, 3H), 3.72-3.55 (m, 6H), 3.45-3.3 (m, 4H), 3.10-2.89 (m, 5H), 2.09 & 2.08 (2s, 3H), 1.40-1.21 (m, 11H), 1.15-1.03 (m, 2H), 0.76 (t, 3H). MS m/z [M+H]⁺ 950.7.

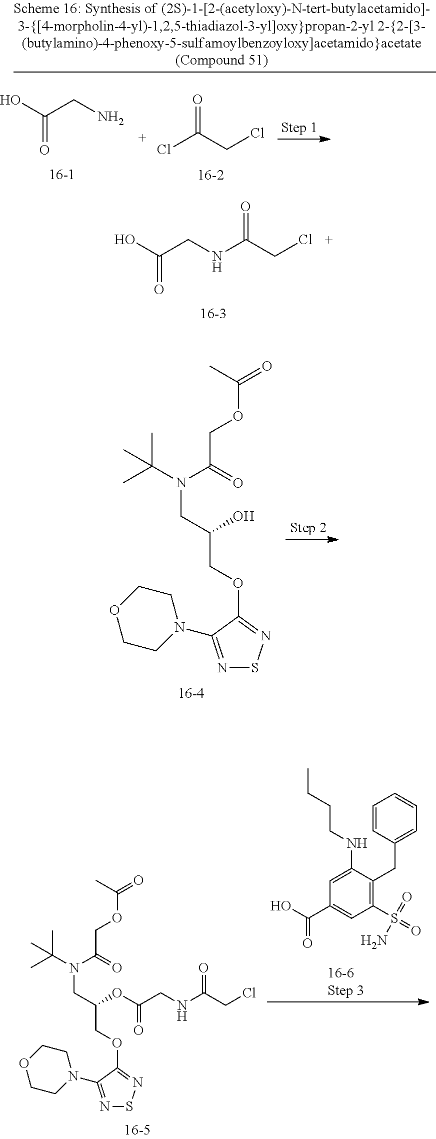 [SMILES: NCC(=O)O ; O=C(Cl)CCl ; O=C(O)CNC(=O)CCl ; CC(=O)OCC(=O)N(C[C@H](O)COc1nsnc1N1CCOCC1)C(C)(C)C ; CC(=O)OCC(=O)N(C[C@@H](COc1nsnc1N1CCOCC1)OC(=O)CNC(=O)CCl)C(C)(C)C ; CCCCNc1cc(C(=O)O)cc(S(N)(=O)=O)c1Cc1ccccc1]

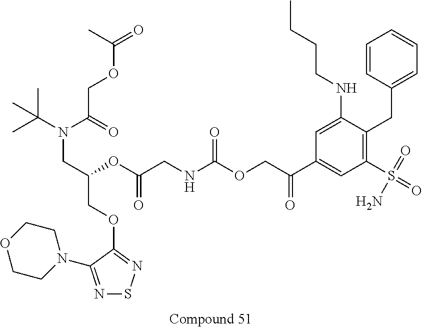 [SMILES: CCCCNc1cc(C(=O)COC(=O)NCC(=O)O[C@H](COc2nsnc2N2CCOCC2)CN(C(=O)COC(C)=O)C(C)(C)C)cc(S(N)(=O)=O)c1Cc1ccccc1]

Step 1: Preparation of (2-chloroacetamido)acetic acid (16-3): To a solution of aminoacetic acid 16-1 (15.0 g, 199.84 mmol) in diethyl ether were added NaOH solution (2.5 N, 75 mL) and chloroacetyl chloride 16-2 (15 mL) slowly at 0-5° C. The reaction mixture was allowed stir at 25-30° C. for 3 h. The resulting reaction mixture was washed with ethyl acetate (250 mL). The aqueous layer was neutralised with 1.0 N HCl (PH=6-7) and extracted with DCM (2×250 mL). The organic layer was dried over sodium sulfate and concentrated under reduced pressure. The crude compound was purified by silica gel (60-120 mesh) column chromatography to obtain 16-3 as a white solid 10.0 g (33%)

Step 2: Preparation of (2S)-1-[2-(acetyloxy)-N-tert-butylacetamido]-3-{[4-(morpholin-4-yl)-1,2,5-thiadiazol-3-yl]oxy}propan-2-yl 2-(2-chloroacetamido)acetate (16-5): To a solution of (2-chloroacetamido)acetic acid 16-3 (1.45 g, 4.8 mmol) in dichloromethane (15 mL) were added DCC (2.17 g, 10.56 mmol), {tert-butyl[(2S)-2-hydroxy-3-{[4-(morpholin-4-yl)-1,2,5-thiadiazol-3-yl]oxy}propyl]carbamoyl}methyl acetate 16-4 (2.0 g, 4.8 mmol) and 4-Dimethylaminopyridine (0.058 g, 0.48 mmol) at 0-5° C. The reaction mixture was allowed to stir at 25-30° C. for 16 h. The resulting reaction mixture was diluted with ethyl acetate (200 mL) and washed with water (100 mL). The organic layer was dried over sodium sulfate and concentrated under reduced pressure at 45° C. The crude compound was purified by silica gel (230-400 mesh) column chromatography to obtain product 16-5 as a colourless wax 0.95 g (35.9%)

Step 3: Preparation of (2S)-1-[2-(acetyloxy)-N-tert-butylacetamido]-3-{[4-(morpholin-4-yl)-1,2,5-thiadiazol-3-yl]oxy}propan-2-yl 2-{2-[3-(butylamino)-4-phenoxy-5-sulfamoylbenzoyloxy]acetamido}acetate (Compound 51): To a solution of bumetanide 16-6 (1.29 g, 3.54 mmol) in N, N-Dimethylformamide (10 mL), were added triethylamine (1.29 mL, 7.08 mmol), NaI (0.58 g, 3.89 mmol) and (2S)-1-[2-(acetyloxy)-N-tert-butylacetamido]-3-{[4-(morpholin-4-yl)-1,2,5-thiadiazol-3-yl]oxy}propan-2-yl2-(2-chloro acetamido)acetate 16-5 (1.95 g, 3.54 mmol) at 0-5° C. The reaction mixture was allowed to stir at 55° C. for 2 h. The resulting reaction mixture was diluted with ethyl acetate (150 mL) and washed with water (2×50 mL). The organic layer was dried over sodium sulfate and concentrated under reduced pressure at 45° C. The crude compound was purified by silica gel (230-400 mesh) column chromatography to obtain product Compound 51 as an off white solid 1.1 g (35%). ¹H-NMR (400 MHz, DMSO-d6) δ 8.60 (t, 1H), 7.74 (d, J=2 Hz, 1H), 7.47-7.37 (m, 3H), 7.27 (t, J=8 Hz, 2H), 7.01 (t, J=8 Hz, 1H), 6.85 (d, J=8 Hz, 2H), 5.46-5.37 (m, 1H), 5.16 (t, 1H), 4.96 (d, 1H), 4.83-4.77 (m, 2H), 4.68 (d, 1H), 4.61-4.52 (m, 1H), 4.50-4.41 (m, 1H), 4.08-3.98 (m, 1H), 3.95-3.87 (m, 1H), 3.70-3.53 (m, 6H), 3.43-3.27 (m, 4H), 3.07 (q, 2H), 2.08 (s, 3H), 1.41-1.21 (m, 11H), 1.15-1.02 (m, 2H), 0.76 (t, 3H). MS m/z [M+H]⁺ 878.7.

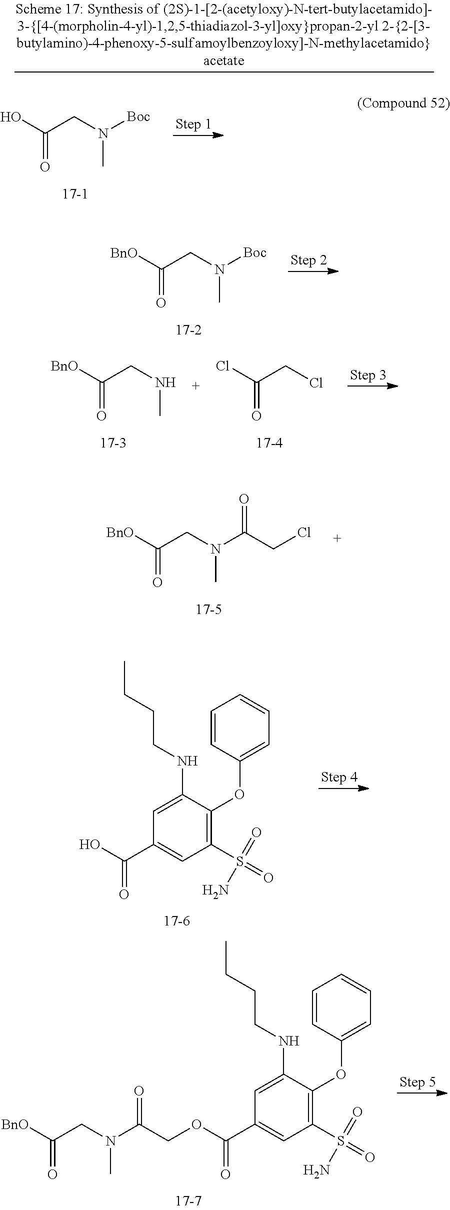

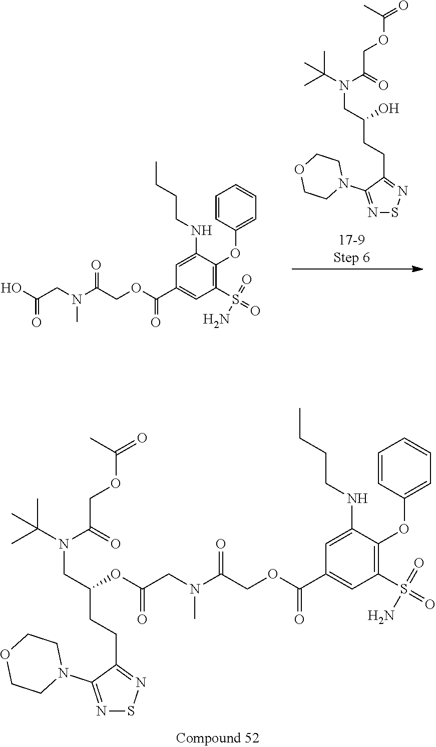 [SMILES: CCCCNc1cc(C(=O)OCC(=O)N(C)CC(=O)O)cc(S(N)(=O)=O)c1Oc1ccccc1 ; CC(=O)OCC(=O)N(C[C@H](O)CCc1nsnc1N1CCOCC1)C(C)(C)C ; CCCCNc1cc(C(=O)OCC(=O)N(C)CC(=O)O[C@H](CCc2nsnc2N2CCOCC2)CN(C(=O)COC(C)=O)C(C)(C)C)cc(S(N)(=O)=O)c1Oc1ccccc1]

Step 1: Preparation of benzyl [(tert-butoxycarbonyl)(methyl)amino]acetate (17-2): To a solution of [(tert-butoxycarbonyl)(methyl)amino]acetic acid 17-1 (50.0 g, 264.0 mmol) in dichloromethane (500 mL) were added EDC.HCl (75.71 g, 396.0 mmol), benzyl alcohol (22.86 g, 211.0 mmol) and 4-Dimethylaminopyridine (3.22 g, 26.0 mol) at 0-5° C. The reaction mixture was allowed to stir at 25-30° C. for 2 h. The resulting reaction mixture was diluted with ethyl acetate (800 mL) and washed with water (300 mL). The organic layer was dried over sodium sulfate and concentrated under reduced pressure at 45° C. The crude compound was purified by silica (60-120 mesh) column chromatography to obtain product 17-2 as a colourless wax 54.0 g (73%).

Step 2: Preparation of benzyl (methylamino)acetate (17-3): To a solution of benzyl [(tert-butoxycarbonyl)(methyl)amino]acetate 17-2 (54.0 g, 193.0 mmol) in dichloromethane (540 mL) was added TFA (216 mL) at 0-5° C. The reaction mixture was allowed to stir at 25-30° C. for 1 h. The resulting reaction mixture was concentrated under reduced pressure at 45° C. to obtain product 17-3 as brown wax 85.0 g (crude compound as a TFA salt was carried as such into next step).

Step 3: Preparation of benzyl [(chloroacetyl)(methyl)amino]acetate (17-5): To a solution of benzyl (methylamino)acetate 17-3 (85.0 g, 474.0 mmol) in dichloromethane (850 mL) were added triethylamine (194.57 mL, 1422 mmol), 4-Dimethylaminopyridine (5.78 g, 47.0 mmol) and chloroacetyl chloride 17-4 (56.56 mL, 711.0 mmol) slowly at 0-5° C. The reaction mixture was allowed to stir at 25-30° C. for 1 h. The resulting reaction mixture was diluted with ethyl acetate (1.2 L) and washed with water (2×500 mL). The organic layer was dried over sodium sulfate and concentrated under reduced pressure at 45° C. The crude compound was purified by silica gel (230-400 mesh) column chromatography to obtain product 17-5 as a colourless wax 23.0 g (18.9%).

Step 4: Preparation of benzyl 2-{2-[3-(butylamino)-4-phenoxy-5-sulfamoylbenzoyloxy]-N-methylacetamido}acetate (17-7): To a solution of bumetanide 17-6 (19 g, 52.13 mmol) in N, N-Dimethylformamide (100 mL) were added K₂CO₃ (8.64 g, 62.55 mmol), TBAI (1.92 g, 5.21 mmol) and benzyl [(chloroacetyl)(methyl)amino]acetate 17-5 (17.33 g, 67.78 mmol) at 0-5° C. The reaction mixture was allowed to stir at 25-30° C. for 4 h. The resulting reaction mixture was diluted with ethyl acetate (400 mL) and washed with water (2×250 mL). The organic layer was dried over sodium sulfate and concentrated under reduced pressure at 45° C. The crude compound was purified by silica gel (230-400 mesh) column chromatography to obtain product 17-7 as an off white solid 21.5 g (69%).

Step 5: Preparation of 2-{2-[3-(butylamino)-4-phenoxy-5-sulfamoylbenzoyloxy]-N-methylacetamido}acetic acid (17-8): 10% Pd/C (4 g, 50% wet, 20% w/w) was added to a solution of to a solution of benzyl 2-{2-[3-(butylamino)-4-phenoxy-5-sulfamoylbenzoyloxy]-N-methylacetamido} acetate 17-7 (21.5 g, 36.83 mmol) in methanol (150 mL) and dichloromethane (45 mL) taken in a Parr-shaker vessel. The reaction mixture was hydrogenated with 5 kg/cm² hydrogen pressure at 25-30° C. for 1 h. The resulting reaction mixture was filtered through celite bed. The filtrate was concentrated under reduced pressure at 45° C. to obtain 17-8 as an off white solid 15.5 g (85.3%).

Step 6: Preparation of 2-{2-[3-(butylamino)-4-phenoxy-5-sulfamoylbenzoyloxy]-N-methylacetamido}acetic acid (Compound 52): To a solution of 2-{2-[3-(butylamino)-4-phenoxy-5-sulfamoylbenzoyloxy]-N-methylacetamido}acetic acid 17-8 (1.82 g, 3.69 mmol) in dichloromethane (20 mL) were added EDC.HCl (0.834 g, 4.37 mmol), HOBt (0.93 g, 0.677 mmol) {tert-butyl[(2S)-2-hydroxy-3-{[4-(morpholin-4-yl)-1,2,5-thiadiazol-3-yl]oxy}propyl]carbamoyl}methyl acetate 17-9 (1.4 g, 3.36 mmol) and 4-Dimethylaminopyridine (0.4 g, 0.33 mol) at 0-5° C. The reaction mixture was allowed to stir at 25-30° C. for 16 h. The resulting reaction mixture was diluted with ethyl acetate (150 mL) and washed with water (75 mL). The organic layer was dried over sodium sulfate and concentrated under reduced pressure at 45° C. The crude compound was purified by silica gel (230-400 mesh) column chromatography to obtain product Compound 52 as an off white solid 0.315 g (10.5%). ¹H-NMR (400 MHz, DMSO-d6) δ 7.75-7.71 (m, 1H), 7.46-7.33 (m, 3H), 7.27 (t, 2H), 7.01 (t, 1H), 6.88-6.82 (m, 2H), 5.6-5.4 (m, 1H), 5.2-4.1 (m, 9H), 3.75-3.53 (m, 6H), 3.43-3.27 (m, 4H), 3.10-3.02 (m, 2H), 2.99 & 2.82 (2s, 3H), 2.11 & 2.10 (2s, 3H), 1.41-1.23 (m, 11H), 1.15-1.03 (m, 2H), 0.76 (t, 3H). MS m/z [M+H]⁺ 892.7.

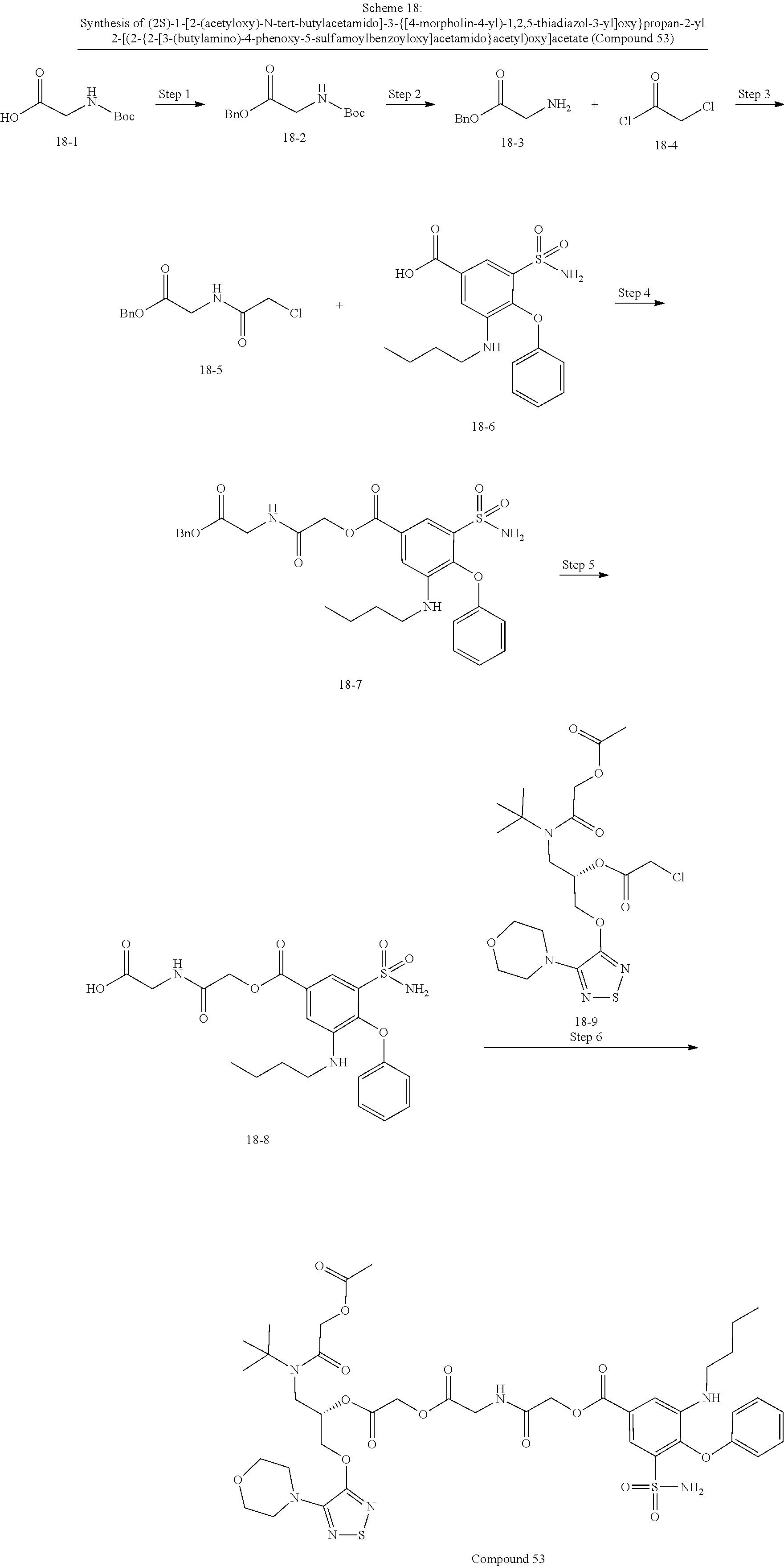

Step 1: Preparation of benzyl [(tert-butoxycarbonyl)amino]acetate (18-2): To a solution of [(tert-butoxycarbonyl) amino]acetic acid 18-1 (35 g, 199.78 mmol) in dichloromethane (50 mL) were added EDC.HCl (57.24 g, 299.6 mmol), benzyl alcohol (17.28 g, 159.82 mmol) and 4-Dimethylaminopyridine (2.43 g, 19.97 mmol) at 0-5° C. The reaction mixture was allowed to stir at 25-30° C. for 1 h. The resulting reaction mixture was diluted with ethyl acetate (800 mL) and washed with water (500 mL). The organic layer was dried over sodium sulfate and concentrated under reduced pressure at 45° C. The crude compound was purified by silica (60-120 mesh) column chromatography to obtain product 18-2 as a colourless wax 52.0 g (98%).

Step 2: Preparation of benzyl aminoacetate (18-3): To a solution of benzyl [(tert-butoxycarbonyl)amino]acetate 18-2 (52.0 g, 196 mmol) in dichloromethane (520 mL) was added TFA (208 mL) at 0-5° C. The reaction mixture was allowed to stir at 25-30° C. for 1 h. The resulting reaction mixture was concentrated under reduced pressure at 45° C. to obtain product 18-3 as a brown wax 70.0 g (crude compound 18-3 as a TFA salt was taken to next step without any purification).

Step 3: Preparation of benzyl (2-chloroacetamido)acetate (18-5): To a solution of benzyl aminoacetate 18-3 (70.0 g, 423.8 mmol) in dichloromethane (700 mL) were added triethylamine (173.8 mL, 1271 mmol), 4-Dimethylaminopyridine (5.17 g, 43.38 mmol) and chloroacetyl chloride 18-4 (33.69 mL, 423.8 mmol) slowly at 0-5° C. The reaction mixture was allowed to stir at 25-30° C. for 1 h. The resulting reaction mixture was diluted with ethyl acetate (1.2 L) and washed with water (2×500 mL). The organic layer was dried over sodium sulfate and concentrated under reduced pressure at 45° C. The crude was purified by silica gel (230-400 mesh) column chromatography to obtain product 18-5 as a colourless wax 19.35 g (18.8%).

Step 4: Preparation of benzyl 2-{2-[3-(butylamino)-4-phenoxy-5-sulfamoylbenzoyloxy]acetamido}acetate (18-7): To a solution of bumetanide 18-6 (30.0 g, 82.32 mmol) in N, N-Dimethylformamide (150 mL) were added triethylamine (28.14 mL, 20.58 mmol), NaI (14.8 g, 98.78 mmol) and benzyl (2-chloroacetamido)acetate 18-5 (23.87 g, 98.78 mmol) at 0-5° C. The reaction mixture was allowed to stir at 25-30° C. for 2 h. The resulting reaction mixture was diluted with ethyl acetate (750 mL) and washed with water (2×250 mL). The organic layer was dried over sodium sulfate and concentrated under reduced pressure at 45° C. The crude was purified by silica gel (230-400 mesh) column chromatography to obtain product 18-7 as an off white solid 19.2 g (40.59%).

Step 5: Preparation of 2-{2-[3-(butylamino)-4-phenoxy-5-sulfamoylbenzoyloxy]acetamido}acetic acid (18-8): 10% Pd/C (2 g, 50% wet, 20% w/w) was added to a solution of to a solution of 2-{2-[3-(butylamino)-4-phenoxy-5-sulfamoylbenzoyloxy]acetamido}acetate 18-7 (10 g, 17.55 mmol) in methanol (70 mL) and dichloromethane (30 mL) taken in a Parr-shaker vessel. The reaction mixture was hydrogenated with 5 kg/cm² hydrogen pressure at 25-30° C. for 1 h. The resulting reaction mixture was filtered through celite bed. The filtrate was concentrated under reduced pressure at 45° C. to obtain product 18-8 as an off white solid 6.0 g (71%).

Step 6: Preparation of (2S)-1-[2-(acetyloxy)-N-tert-butylacetamido]-3-{[4-(morpholin-4-yl)-1,2,5-thiadiazol-3-yl]oxy}propan-2-yl 2-[(2-{2-[3-(butylamino)-4-phenoxy-5-sulfamoylbenzoyloxy]acetamido}acetyl)oxy]acetate (Compound 53): To a solution of 2-{2-[3-(butylamino)-4-phenoxy-5-sulfamoylbenzoyloxy]acetamido}acetic acid (18-8) (2.2 g, 4.59 mmol) in N, N-Dimethylformamide (15 mL), were added triethylamine (1.25 mL, 9.18 mmol), NaI (0.825 g, 5.5 mmol) and (2S)-1-[2-(acetyloxy)-N-tert-butylacetamido]-3-{[4-(morpholin-4-yl)-1,2,5-thiadiazol-3-yl]oxy}propan-2-yl 2-chloroacetate 18-9 (2.94 g, 5.97 mmol) 0-5° C. The reaction mixture was allowed to stir at 55° C. for 16 h. The resulting reaction mixture was diluted with ethyl acetate (200 mL) and washed with water (2×75 mL). The organic layer was dried over sodium sulfate and concentrated under reduced pressure at 45° C. The crude was purified by silica gel (230-400 mesh) column chromatography to obtain product Compound 53 as an off white solid 1.1 g (25.6%). ¹H-NMR (400 MHz, DMSO-d6) δ 8.67 (t, 1H), 7.75 (d, J=2 Hz, 1H), 7.47-7.37 (m, 3H), 7.27 (t, J=8 Hz, 2H), 7.02 (t, J=8 Hz, 1H), 6.85 (d, J=8 Hz, 2H), 5.51-5.40 (m, 1H), 5.15 (t, 1H), 4.92 (d, 1H), 4.87-4.73 (m, 4H), 4.68 (d, 1H), 4.64-4.55 (m, 1H), 4.50-4.41 (m, 1H), 4.09-3.94 (m, 2H), 3.71-3.54 (m, 6H), 3.43-3.3 (m, 4H), 3.07 (q, 2H), 2.09 (s, 3H), 1.42-1.23 (m, 11H), 1.15-1.02 (m, 2H), 0.76 (t, 3H). MS m/z [M+H]⁺ 936.7.

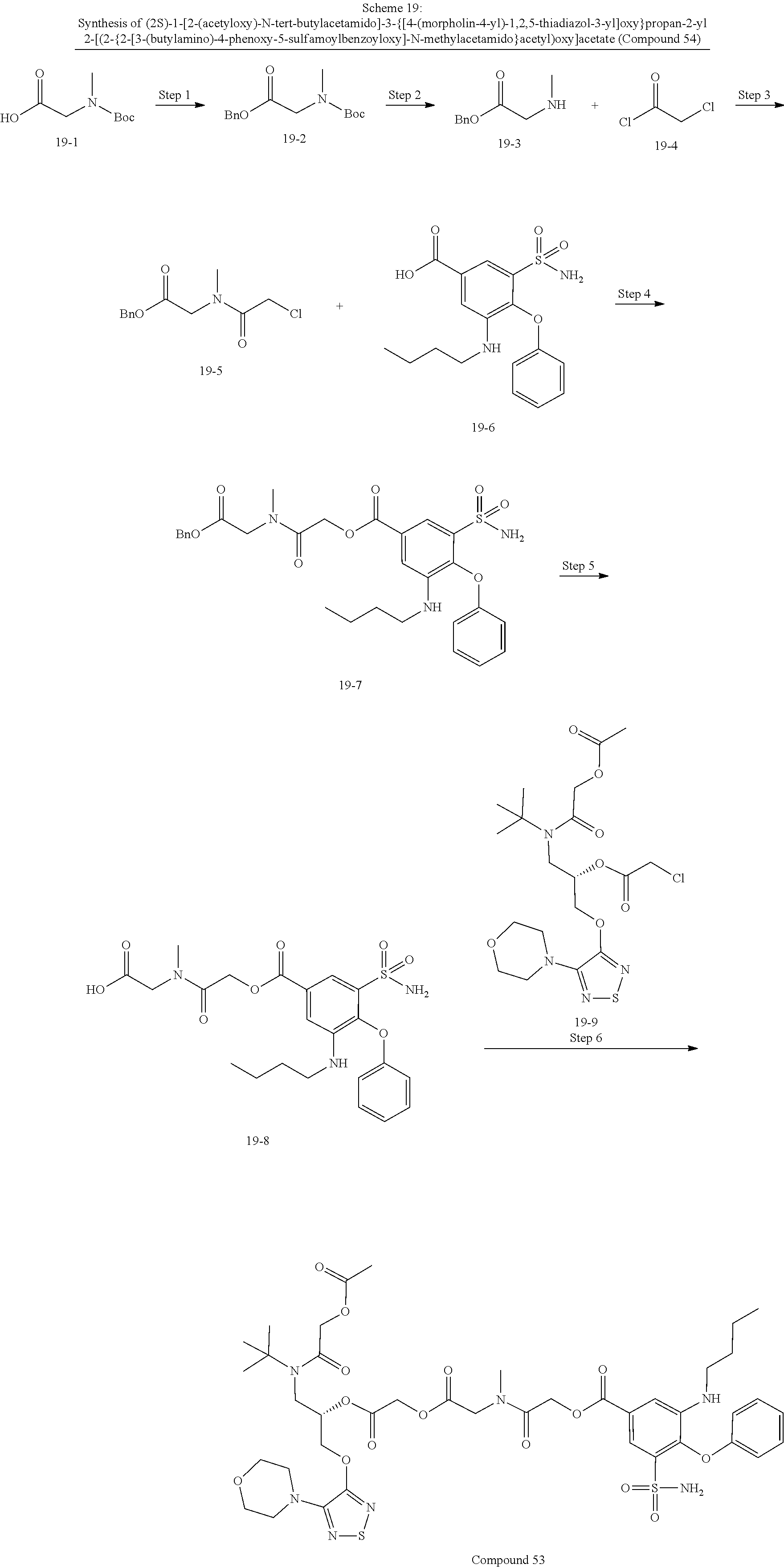

Step 1: Preparation of benzyl [(tert-butoxycarbonyl)(methyl)amino]acetate (19-2):

To a solution of [(tert-butoxycarbonyl)(methyl)amino]acetic acid 19-1 (50.0 g, 264.0 mmol) in dichloromethane (500 mL) were added EDC.HCl (75.71 g, 396.0 mmol), benzyl alcohol (22.86 g, 211.0 mmol) and 4-Dimethylaminopyridine (3.22 g, 26.0 mol) at 0-5° C. The reaction mixture was allowed to stir at 25-30° C. for 2 h. The resulting reaction mixture was diluted with ethyl acetate (800 mL) and washed with water (300 mL). The organic layer was dried over sodium sulfate and concentrated under reduced pressure at 45° C. The crude compound was purified by silica (60-120 mesh) column chromatography to obtain product 19-2 as a colourless wax 54.0 g (73%).

Step 2: Preparation of benzyl (methylamino)acetate (19-3): To a solution of benzyl [(tert-butoxycarbonyl)(methyl)amino]acetate 19-2 (54.0 g, 193.0 mmol) in dichloromethane (540 mL) was added TFA (216 mL) at 0-5° C. The reaction mixture was allowed to stir at 25-30° C. for 1 h. The resulting reaction mixture was concentrated under reduced pressure at 45° C. to obtain product 19-3 as brown colour wax 85.0 g (crude compound 19-3 as a TFA salt was taken as such into next step).

Step 3: Preparation of benzyl [(chloroacetyl)(methyl)amino]acetate (19-5): To a solution of benzyl (methylamino)acetate 19-3 (85.0 g, 474.0 mmol) in dichloromethane (850 mL) were added triethylamine (194.57 mL, 1422 mmol), 4-Dimethylaminopyridine (5.78 g, 47.0 mmol) and chloro acetyl chloride 19-4 (56.56 mL, 711.0 mmol) slowly at 0-5° C. The reaction mixture was allowed to stir at 25-30° C. for 1 h. The resulting reaction mixture was diluted with ethyl acetate (1.2 L) and washed with water (2×500 mL). The organic layer was dried over sodium sulfate and concentrated under reduced pressure at 45° C. The crude compound was purified by silica gel (230-400 mesh) column chromatography to obtain product 19-5 as a colourless wax 23.0 g (18.9%).

Step 4: Preparation of benzyl 2-{2-[3-(butylamino)-4-phenoxy-5-sulfamoylbenzoyloxy]-N-methylacetamido}acetate (19-7): To a solution of bumetanide 19-6 (19 g, 52.13 mmol) in N, N-Dimethylformamide (100 mL) were added K₂CO₃ (8.64 g, 62.55 mmol), TBAI (1.92 g, 5.21 mmol) and benzyl [(chloroacetyl)(methyl)amino]acetate 19-5 (17.33 g, 67.78 mmol) at 0-5° C. The reaction mixture was allowed to stir at 25-30° C. for 4 h. The resulting reaction mixture was diluted with ethyl acetate (400 mL) and washed with water (2×250 mL). The organic layer was dried over sodium sulfate and concentrated under reduced pressure at 45° C. The crude compound was purified by silica gel (230-400 mesh) column chromatography to obtain product 19-7 as an off white solid 21.5 g (69%).

Step 5: Preparation of 2-{2-[3-(butylamino)-4-phenoxy-5-sulfamoylbenzoyloxy]-N-methylacetamido}acetic acid (19-8): 10% Pd/C (4 g, 50% wet, 20% w/w) was added to a solution of to a solution of benzyl 2-{2-[3-(butylamino)-4-phenoxy-5-sulfamoylbenzoyloxy]-N-methylacetamido} acetate 19-7 (21.5 g, 36.83 mmol) in methanol (150 mL) and dichloromethane (45 mL) taken in a Parr-shaker vessel. The reaction mixture was hydrogenated with 5 kg/cm² hydrogen pressure at 25-30° C. for 1 h. The resulting reaction mixture was filtered through celite bed. The filtrate was concentrated under reduced pressure at 45° C. to obtain 19-8 as an off white solid 15.5 g (85.3%).

Step 6: Preparation of (2S)-1-[2-(acetyloxy)-N-tert-butylacetamido]-3-{[4-(morpholin-4-yl)-1,2,5-thiadiazol-3-yl]oxy}propan-2-yl 2-[(2-{2-[3-(butylamino)-4-phenoxy-5-sulfamoylbenzoyloxy]-N-methylacetamido}acetyl)oxy]acetate (Compound 53): To a solution of 2-{2-[3-(butylamino)-4-phenoxy-5-sulfamoylbenzoyloxy]-N-methylacetamido}acetic acid 19-8 (2.2 g, 4.46 mmol) in N, N-Dimethylformamide (20 mL), were added triethylamine (1.22 mL, 8.92 mmol), NaI (0.8 g, 5.35 mmol) and (2S)-1-[2-(acetyloxy)-N-tert-butylacetamido]-3-{[4-(morpholin-4-yl)-1,2,5-thiadiazol-3-yl]oxy}propan-2-yl 2-chloroacetate 19-9 (2.64 g, 5.35 mmol) at 0-5° C. The reaction mixture was allowed to stir at 55° C. for 3 h. The resulting reaction mixture was diluted with ethyl acetate (200 mL) and washed with water (2×60 mL). The organic layer was dried over sodium sulfate and concentrated under reduced pressure at 45° C. The crude compound was purified by silica gel (230-400 mesh) column chromatography to obtain product Compound 53 as an off white solid 2.6 g (59.3%). ¹H-NMR (400 MHz, DMSO-d6) δ 7.76-7.71 (m, 1H), 7.46-7.37 (m, 3H), 7.27 (t, 2H), 7.01 (t, 1H), 6.88-6.82 (m, 2H), 5.52-5.41 (m, 1H), 5.22-4.23 (m, 11H), 3.71-3.54 (m, 6H), 3.43-3.3 (m, 4H), 3.10-2.85 (m, 5H), 2.10 & 2.09 (2s, 3H), 1.41-1.23 (m, 11H), 1.15-1.03 (m, 2H), 0.76 (t, 3H). MS m/z [M+H]⁺ 950.6.

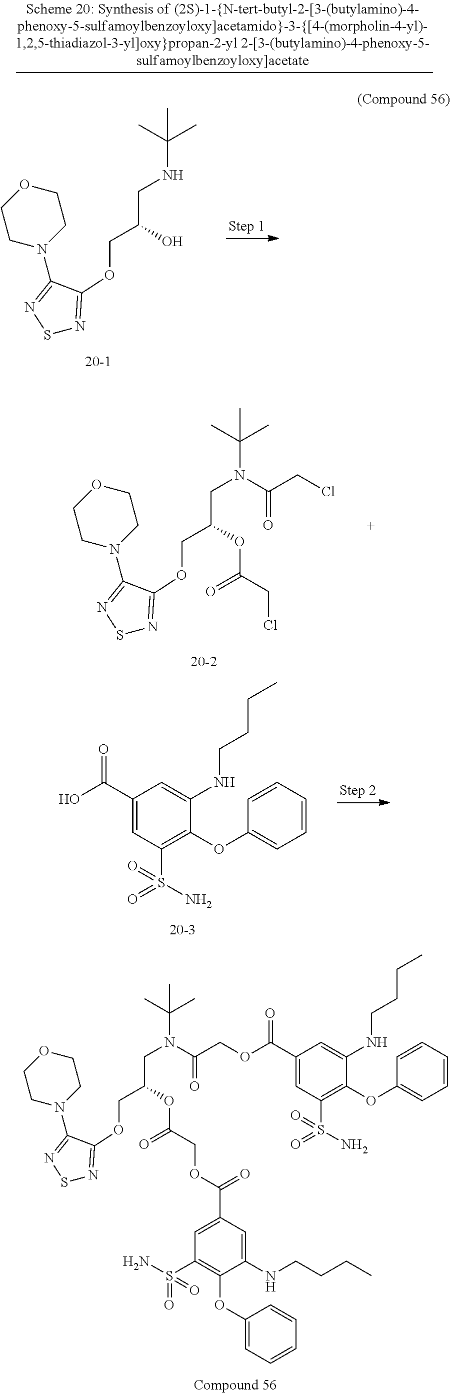 [SMILES: CC(C)(C)NC[C@H](O)COc1nsnc1N1CCOCC1 ; CC(C)(C)N(C[C@@H](COc1nsnc1N1CCOCC1)OC(=O)CCl)C(=O)CCl ; CCCCNc1cc(C(=O)O)cc(S(N)(=O)=O)c1Oc1ccccc1 ; CCCCNc1cc(C(=O)OCC(=O)O[C@H](COc2nsnc2N2CCOCC2)CN(C(=O)COC(=O)c2cc(NCCCC)c(Oc3ccccc3)c(S(N)(=O)=O)c2)C(C)(C)C)cc(S(N)(=O)=O)c1Oc1ccccc1]

Step 1: Preparation of (2S)-1-(N-tert-butyl-2-chloroacetamido)-3-{[4-(morpholin-4-yl)-1,2,5-thiadiazol-3-yl]oxy}propan-2-yl 2-chloroacetate (20-2): To a solution of timolol 20-1 (10.0 g, 31.6 mmol) in 2-methyl THF (100 mL) was added triethylamine (34.56 mL, 252.8 mmol) and chloroacetyl chloride (12.56 mL, 158 mmol) drop-wise at −30 to −20° C. The reaction mixture was allowed to stir at 25-30° C. for 16 h. The resulting the reaction mixture was diluted with ethyl acetate (500 mL) and washed with water (2×200 mL). The organic layer was dried over sodium sulfate and concentrated under reduced pressure at 45° C. The crude compound was purified by silica gel (230-400 mesh) column chromatography to obtain 20-2 as a colourless wax 10.5 g (70.8%)

Step 2: Preparation of (2S)-1-{N-tert-butyl-2-[3-(butylamino)-4-phenoxy-5-sulfamoylbenzoyloxy]acetamido}-3-{[4-(morpholin-4-yl)-1,2,5-thiadiazol-3-yl]oxy}propan-2-yl 2-[3-(butylamino)-4-phenoxy-5-sulfamoylbenzoyloxy]acetate (Compound 56): To a solution of bumetanide 20-3 (2.94 g, 8.08 mmol) in N, N-Dimethylformamide (20 mL) were added K₂CO₃ (1.23 g, 8.88 mmol), TBAI (0.15 g, 0.4 mmol) and (2S)-1-(N-tert-butyl-2-chloroacetamido)-3-{[4-(morpholin-4-yl)-1,2,5-thiadiazol-3-yl]oxy}propan-2-yl 2-chloroacetate 20-2 (1.9 g, 4.04 mmol) at 0-5° C. The reaction mixture was allowed to stir at 25-30° C. for 16 h. The resulting reaction mixture was diluted with ethyl acetate (250 mL) and washed with water (2×100 mL). The organic layer was dried over sodium sulfate and concentrated under reduced pressure at 45° C. The crude compound was purified by silica gel (230-400 mesh) column chromatography to obtain product Compound 56 as an off white solid 1.6 g (35%). ¹H-NMR (400 MHz, DMSO-d6) δ 7.79-7.71 (m, 2H), 7.50-7.35 (m, 6H), 7.32-7.22 (m, 4H), 7.004-6.96 (m, 2H), 6.88-6.82 (m, 4H), 5.62-5.51 (m, 1H), 5.35-4.98 (m, 6H), 4.80-4.71 (m, 1H), 4.58-4.49 (m, 1H), 3.80-3.60 (m, 6H), 3.48-3.3 (m, 4H), 3.10-2.97 (m, 4H), 1.42-1.27 (m, 13H), 1.13-0.99 (m, 4H), 0.78-0.69 (m, 6H). MS m/z [M+H]⁺ 1126.5.

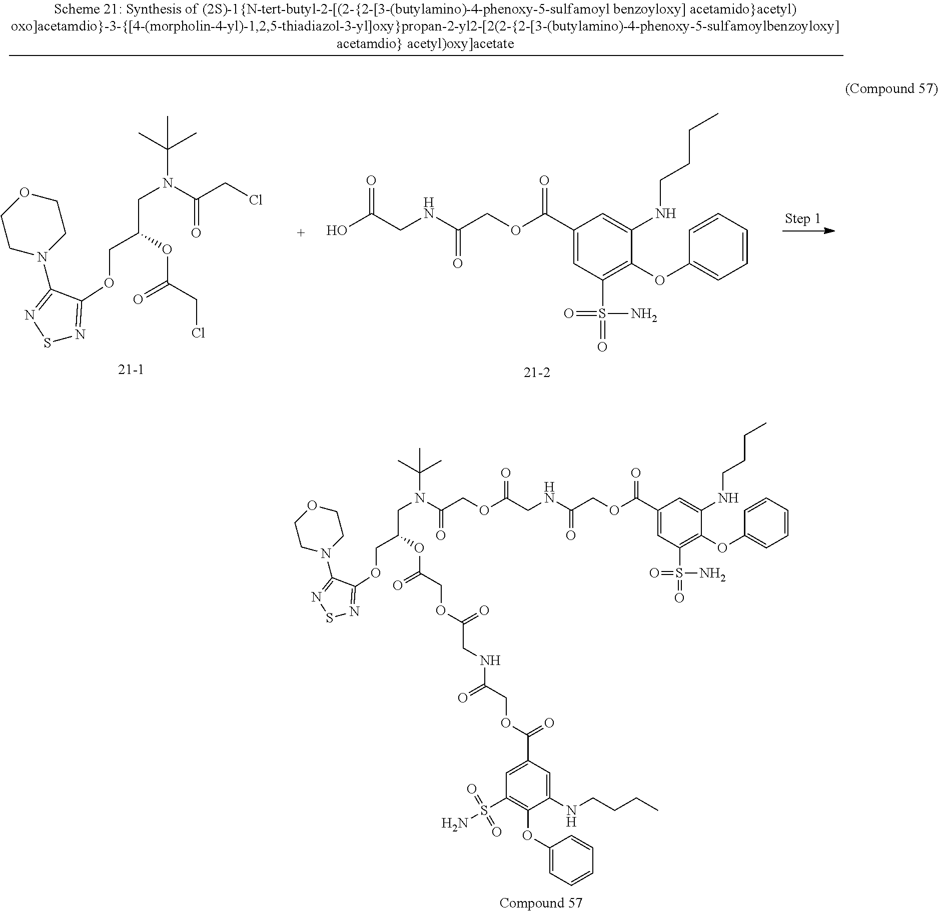 [SMILES: CC(C)(C)N(C[C@@H](COc1nsnc1N1CCOCC1)OC(=O)CCl)C(=O)CCl ; CCCCNc1cc(C(=O)OCC(=O)NCC(=O)O)cc(S(N)(=O)=O)c1Oc1ccccc1 ; CCCCNc1cc(C(=O)OCC(=O)NCC(=O)OCC(=O)O[C@H](COc2nsnc2N2CCOCC2)CN(C(=O)COC(=O)CNC(=O)COC(=O)c2cc(NCCCC)c(Oc3ccccc3)c(S(N)(=O)=O)c2)C(C)(C)C)cc(S(N)(=O)=O)c1Oc1ccccc1]

Step 1: Preparation of (2S)-1-{N-tert-butyl-2-[(2-{2-[3-(butylamino)-4-phenoxy-5-sulfamoylbenzoyloxy]acetamido}acetyl)oxy]acetamido}-3-{[4-(morpholin-4-yl)-1,2,5-thiadiazol-3-yl]oxy}propan-2-yl 2-[(2-{2-[3-(butylamino)-4-phenoxy-5-sulfamoylbenzoyloxy]acetamido}acetyl)oxy]acetate (Compound 57): To a solution of 2-{2-[3-(butylamino)-4-phenoxy-5-sulfamoylbenzoyloxy]acetamido}acetic acid 21-2 (3.97 g, 8.31 mmol) in N,N-Dimethylformamide (25 mL) were added triethylamine (4.03 mL, 7.97 mmol), NaI (0.956 g, 6.38 mmol) and (2S)-1-(N-tert-butyl-2-chloroacetamido)-3-{[4-(morpholin-4-yl)-1,2,5-thiadiazol-3-yl]oxy}propan-2-yl 2-chloroacetate 21-1 (1.5 g, 3.19 mmol) at 0-5° C. The reaction mixture was allowed to stir at 25-30° C. for 16 h. The resulting reaction mixture was diluted with ethyl acetate (300 mL) and washed with water (2×150 mL). The organic layer was dried over sodium sulfate and concentrated under reduced pressure at 45° C. The crude compound was purified by silica gel (230-400 mesh) column chromatography to obtain product Compound 57 as a pale yellow solid 1.05 g (24.2%). ¹H-NMR (400 MHz, DMSO-d6) δ 8.77-8.64 (m, 2H), 7.75 (d, 2H), 7.49-7.33 (m, 6H), 7.27 (t, J=8 Hz, 4H), 6.99 (t, J=8 Hz, 2H), 6.85 (d, J=8 Hz, 4H), 5.51-5.42 (m, 1H), 5.14 (t, 2H), 4.99 (d, 1H), 4.86-4.74 (m, 7H), 4.65-4.55 (m, 1H), 4.50-4.40 (m, 1H), 4.10-3.94 (m, 4H), 3.75-3.55 (m, 6H), 3.44-3.3 (m, 4H), 3.10-3.01 (m, 4H), 1.42-1.20 (m, 13H), 1.16-0.99 (m, 4H), 0.76 (t, 6H). MS m/z [M−H]⁻ 1355.3.

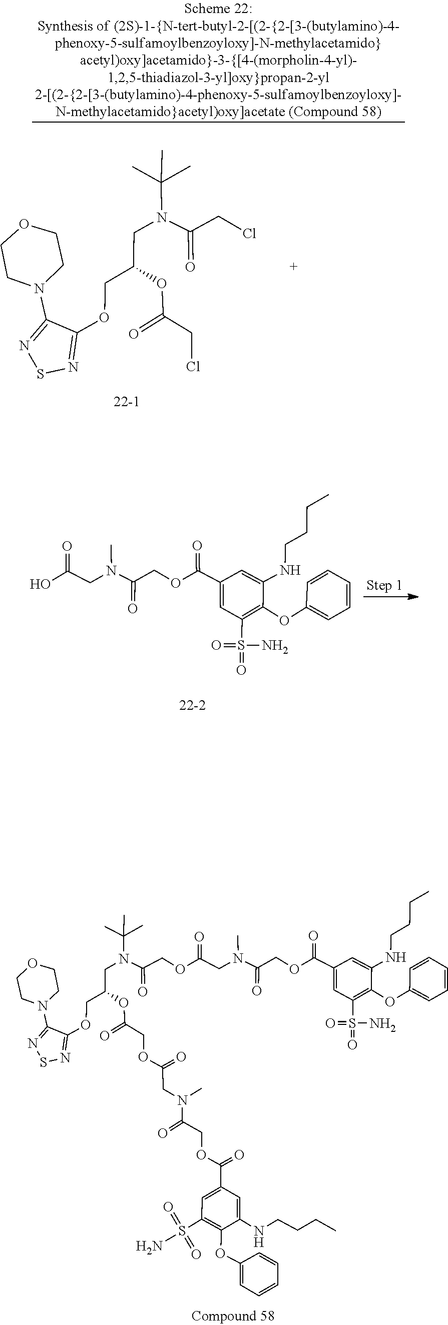 [SMILES: CC(C)(C)N(C[C@@H](COc1nsnc1N1CCOCC1)OC(=O)CCl)C(=O)CCl ; CCCCNc1cc(C(=O)OCC(=O)N(C)CC(=O)O)cc(S(N)(=O)=O)c1Oc1ccccc1 ; CCCCNc1cc(C(=O)OCC(=O)N(C)CC(=O)OCC(=O)O[C@H](COc2nsnc2N2CCOCC2)CN(C(=O)COC(=O)CN(C)C(=O)COC(=O)c2cc(NCCCC)c(Oc3ccccc3)c(S(N)(=O)=O)c2)C(C)(C)C)cc(S(N)(=O)=O)c1Oc1ccccc1]

Step 1: Preparation of (2S)-1-{N-tert-butyl-2-[(2-{2-[3-(butylamino)-4-phenoxy-5-sulfamoylbenzoyloxy]-N-methylacetamido}acetyl)oxy]acetamido}-3-{[4-(morpholin-4-yl)-1,2,5-thiadiazol-3-yl]oxy}propan-2-yl 2-[(2-{2-[3-(butylamino)-4-phenoxy-5-sulfamoylbenzoyloxy]-N-methylacetamido}acetyl)oxy]acetate (Compound 58):

To a solution of 2-{2-[3-(butylamino)-4-phenoxy-5-sulfamoylbenzoyloxy]-N-methylacetamido}acetic acid 22-2 (2.46 g, 4.98 mmol) in N,N-Dimethylformamide (20 mL) were added triethylamine (0.65 mL, 4.77 mmol), NaI (0.63 g, 42.02 mmol) and 2-{2-[3-(butylamino)-4-phenoxy-5-sulfamoylbenzoyloxy]-N-methylacetamido}acetic acid 22-1 (0.9 g, 3.19 mmol) at 0-5° C. The reaction mixture was allowed to stir at 25-30° C. for 16 h. The resulting reaction mixture was diluted with ethyl acetate (150 mL) and washed with water (2×80 mL). The organic layer was dried over sodium sulfate and concentrated under reduced pressure at 45° C. The crude compound was purified by silica gel (230-400 mesh) column chromatography to obtain product Compound 58 as a pale yellow solid 1.2 g (45.2%). ¹H-NMR (400 MHz, DMSO-d6) δ 7.73 (d, 2H), 7.46-7.35 (m, 6H), 7.26 (t, J=8 Hz, 4H), 6.99 (t, J=8 Hz, 2H), 6.85 (d, J=8 Hz, 4H), 5.54-5.42 (m, 1H), 5.20-4.75 (m, 10H), 4.66-4.2 (m, 6H), 3.73-3.56 (m, 6H), 3.44-3.3 (m, 4H), 3.11-2.8 (m, 10H), 1.4-1.3 (m, 13H), 1.16-1.01 (m, 4H), 0.76 (t, 6H). MS m/z [M−H]⁻ 1382.6.

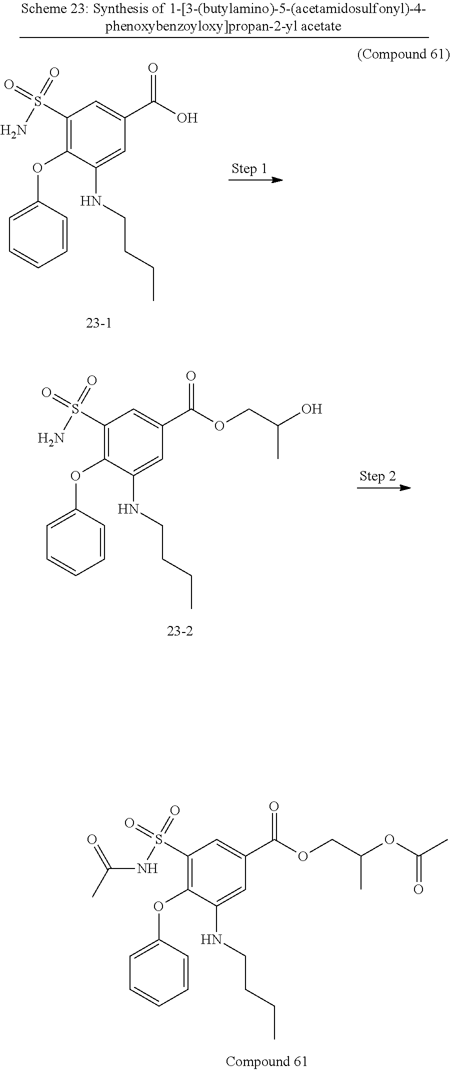 [SMILES: CCCCNc1cc(C(=O)O)cc(S(N)(=O)=O)c1Oc1ccccc1 ; CCCCNc1cc(C(=O)OCC(C)O)cc(S(N)(=O)=O)c1Oc1ccccc1 ; CCCCNc1cc(C(=O)OCC(C)OC(C)=O)cc(S(=O)(=O)NC(C)=O)c1Oc1ccccc1]

Step 1: Preparation of 2-hydroxypropyl 3-(butylamino)-4-phenoxy-5-sulfamoylbenzoate (23-2): To a solution of bumetanide 23-1 (5.0 g, 13.73 mmol) in THF (50 mL) were added EDC.HCl (3.9 g, 20.5 mmol), HOBt (5.2 g, 13.7 mmol), propylene glycol (1.35 g, 17.8 mmol) and 4-Dimethylaminopyridine (0.3 g, 2.74 mmol) at 0-5° C. The reaction mixture was refluxed at 80° C. for 16 h. The resulting reaction mixture was diluted with ethyl acetate (300 mL) and washed with water (2×150 mL). The organic layer was dried over sodium sulfate and concentrated under reduced pressure at 45° C. The crude compound was purified by reverse phase column chromatography to obtain product 23-2 as white solid 2.5 g (43%).

Step 2: Preparation of 1-[3-(butylamino)-5-(acetamidosulfonyl)-4-phenoxybenzoyloxy]propan-2-yl acetate (Compound 61): To a solution of 2-hydroxypropyl 3-(butylamino)-4-phenoxy-5-sulfamoylbenzoate 23-2 (0.9 g, 2.13 mmol) in DCM (10 mL) was added triethylamine (1.84 mL, 12.78 mmol) and acetyl chloride (0.456 mL, 6.39 mmol) drop-wise at 0-5° C. The reaction mixture was allowed to stir at 0-5° C. over a period of 2 h. The crude compound obtained upon evaporation of volatiles was purified by reverse phase column chromatography to obtain product Compound 61 as low melting solid 180 mg (16%). ¹H NMR (400 MHz, DMSO-d₆) δ 12.06 (s, 1H), 7.75-7.70 (m, 1H), 7.48-7.44 (m, 1H), 7.30 (t, J=8 Hz, 2H), 7.05 (t, J=8 Hz, 1H), 6.78 (d, J=8 Hz, 2H), 5.36-5.14 (m, 2H), 4.51-4.16 (m, 2H), 3.10-2.99 (m, 2H), 2.04 & 2.02 (2s, 3H), 1.56 & 1.55 (2s, 3H), 1.41-1.24 (m, 5H), 1.09 (sextet, 2H), 0.77 (t, 3H); MS m/z [M+H]⁺ 507.4.

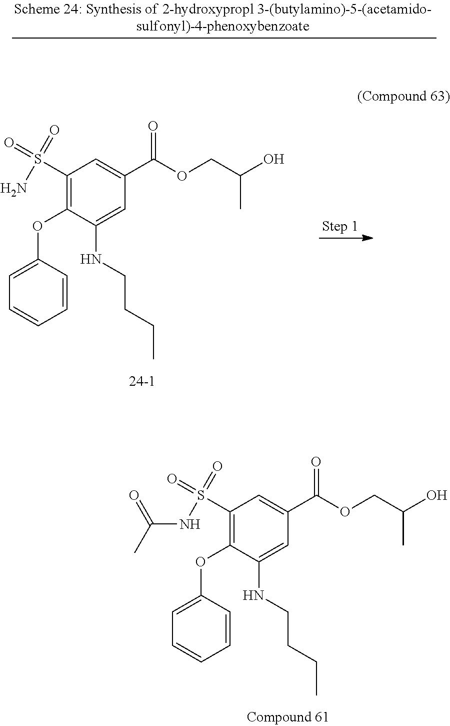 [SMILES: CCCCNc1cc(C(=O)OCC(C)O)cc(S(N)(=O)=O)c1Oc1ccccc1 ; CCCCNc1cc(C(=O)OCC(C)O)cc(S(=O)(=O)NC(C)=O)c1Oc1ccccc1]

Step 1: Preparation of 2-hydroxypropyl 3-(butylamino)-5-(acetamidosulfonyl)-4-phenoxybenzoate (Compound 63): To a solution of 2-hydroxypropyl 3-(butylamino)-4-phenoxy-5-sulfamoylbenzoate 24-1 (1.2 g, 2.84 mmol) in DCM (15 mL) was added triethylamine (0.819 mL, 5.68 mmol) and acetyl chloride (0.20 mL, 2.84 mmol) drop-wise at 0-5° C. The reaction mixture was allowed to stir at 0-5° C. over a period of 2 h. The crude product obtained upon evaporation of volatiles was purified through reverse phase preparative HPLC to obtain product Compound 64 as a white solid 225 mg (17%). ¹H NMR (400 MHz, DMSO-d₆) δ 12.0 (bs, 1H), 7.79-7.74 (m, 1H), 7.48 (s, 1H), 7.29 (t, J=8 Hz, 2H), 7.04 (t, J=8 Hz, 1H), 6.77 (d, J=8 Hz, 2H), 5.26-5.12 (m, 1H), 5.00 (d, 1H), 4.19-4.12 (m, 2H), 4.03-3.93 (m, 1H), 3.05 (q, 2H), 1.51 (s, 3H), 1.35 (quintet, 2H), 1.19-1.02 (m, 5H), 0.76 (t, 3H); MS m/z [M+H]⁺ 465.4.

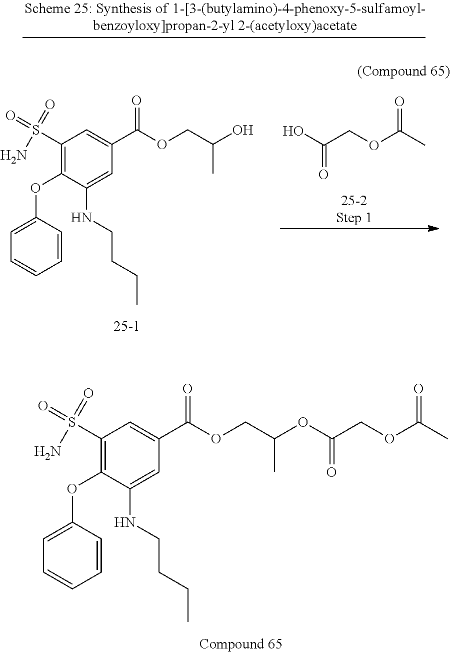

Step 1: Preparation of 1-[3-(butylamino)-4-phenoxy-5-sulfamoylbenzoyloxy]propan-2-yl 2-(acetyloxy)acetate (Compound 65): To a solution of 2-(acetyloxy)acetic acid 25-2 (0.522 g, 4.42 mmol) in THF (10 mL) were added EDC.HCl (1.15 g, 6.03 mmol), hydroxyl benzotriazole (0.547 g, 4.03 mmol), 2-hydroxypropyl 3-(butylamino)-4-phenoxy-5-sulfamoylbenzoate 25-1 (1.7 g, 4.03 mmol) and 4-Dimethylaminopyridine (98 mg, 0.08 mmol) at 0-5° C. The reaction mixture was allowed to stir at 80° C. over a period of 16 h. The crude product obtained upon evaporation of volatiles was purified through reverse phase preparative HPLC to obtain product Compound 65 as white solid 475 mg (22%). ¹H NMR (400 MHz, DMSO-d₆) δ 7.69-7.66 (m, 1H), 7.43-7.35 (m, 3H), 7.27 (t, J=8 Hz, 2H), 7.01 (t, J=8 Hz, 1H), 6.84 (d, J=8 Hz, 2H), 5.34-5.23 (m, 1H), 5.20-5.10 (m, 1H), 4.69-4.62 (m, 2H), 4.47-4.26 (m, 2H), 3.07 (q, 2H), 2.05 (s, 3H), 1.42-1.26 (m, 5H), 1.11 (sextet, 2H), 0.78 (t, 3H); MS m/z [M+H]⁺ 523.7.

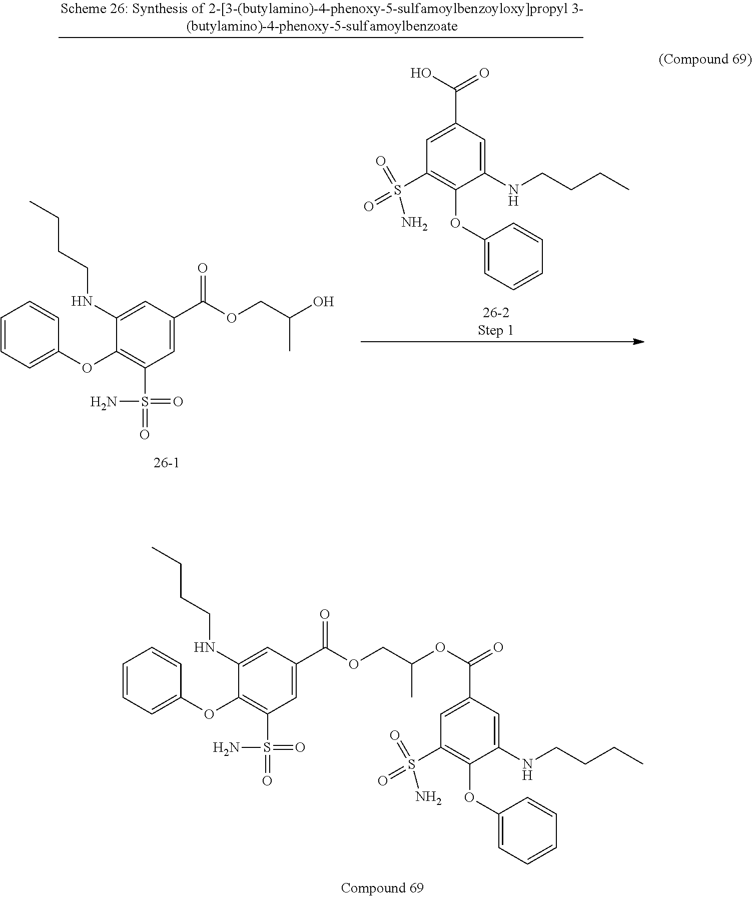 [SMILES: CCCCNc1cc(C(=O)OCC(C)O)cc(S(N)(=O)=O)c1Oc1ccccc1 ; CCCCNc1cc(C(=O)O)cc(S(N)(=O)=O)c1Oc1ccccc1 ; CCCCNc1cc(C(=O)OCC(C)OC(=O)c2cc(NCCCC)c(Oc3ccccc3)c(S(N)(=O)=O)c2)cc(S(N)(=O)=O)c1Oc1ccccc1]

Step 1: Preparation of 2-[3-(butylamino)-4-phenoxy-5-sulfamoylbenzoyloxy]propyl 3-(butylamino)-4-phenoxy-5-sulfamoylbenzoate (Compound 69): To a solution of bumetanide 26-2 (2.80 g, 7.69 mmol) in THF (30 mL) were added EDC.HCl (1.69 g, 8.87 mmol), hydroxyl benzotriazole (0.804 g, 5.91 mmol) 2-hydroxypropyl 3-(butylamino)-4-phenoxy-5-sulfamoylbenzoate 26-1 (2.5 g, 5.91 mmol) and 4-Dimethylaminopyridine (0.144 mg, 1.18 mmol) at 0-5° C. The reaction mixture was allowed to stir at 80° C. over a period of 16 h. The crude product obtained upon evaporation of volatiles was purified through reverse phase column chromatography to obtain product Compound 69 as white solid 1.2 g (26%). ¹H NMR (400 MHz, DMSO-d₆) δ 7.70 (d, J=2 Hz, 2H), 7.43-7.28 (m, 6H), 7.24 (t, J=8 Hz, 4H), 6.98 (t, J=8 Hz, 2H), 6.83 (d, J=8 Hz, 4H), 5.52-5.43 (m, 1H), 5.19-5.08 (m, 2H), 4.66-4.58 (m, 1H), 4.47-4.40 (m, 1H), 3.05-2.92 (m, 4H), 1.43 (d, 3H), 1.37-1.23 (m, 4H), 1.11-0.97 (m, 4H), 0.77-0.65 (m, 6H); MS m/z [M+H]⁺ 769.6.

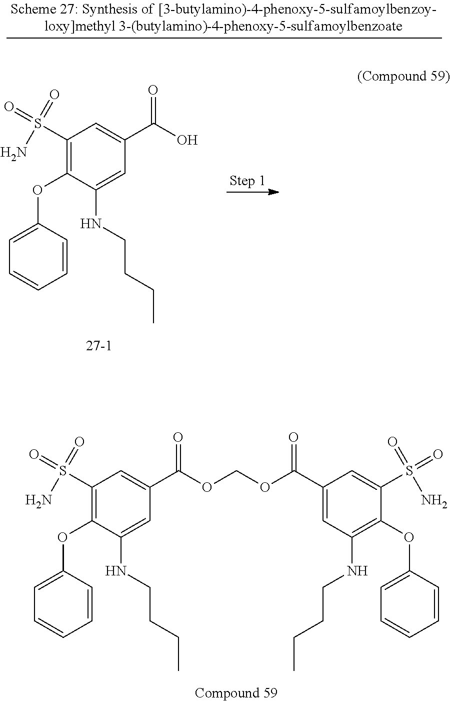 [SMILES: CCCCNc1cc(C(=O)O)cc(S(N)(=O)=O)c1Oc1ccccc1 ; CCCCNc1cc(C(=O)OCOC(=O)c2cc(NCCCC)c(Oc3ccccc3)c(S(N)(=O)=O)c2)cc(S(N)(=O)=O)c1Oc1ccccc1]

Step 1: Preparation of (Compound 59): To a solution of bumetanide 27-1 (4.19 g, 11.50 mmol) in N,N-Dimethylformamide (30 mL) were added K₂CO₃ (1.587 g, 11.50 mmol), TBAI (0.424 g, 1.15 mmol) and dibromomethane (1.0 g, 5.7 mmol) at 0-5° C. The reaction mixture was allowed to stir at 25-30° C. for 16 h. The resulting reaction mixture was diluted with ethyl acetate (200 mL), washed with water (2×80 mL), dried over sodium sulfate and concentrated under reduced pressure. The crude product obtained upon evaporation of volatiles was purified through silica gel (230-400 mesh) column chromatography to obtain product Compound 59 as a white solid 1.1 g (13.5%). ¹H NMR (400 MHz, DMSO-d₆) δ 7.73 (d, J=2 Hz, 2H), 7.47-7.39 (m, 6H), 7.26 (t, J=8 Hz, 4H), 7.01 (t, J=8 Hz, 2H), 6.84 (d, J=8 Hz, 4H), 6.25 (s, 2H), 5.26 (t, 2H), 3.07 (q, 4H), 1.36 (quintet, 4H), 1.10 (sextet, 4H), 0.76 (t, 6H); MS m/z [M+H]⁺ 741.5.

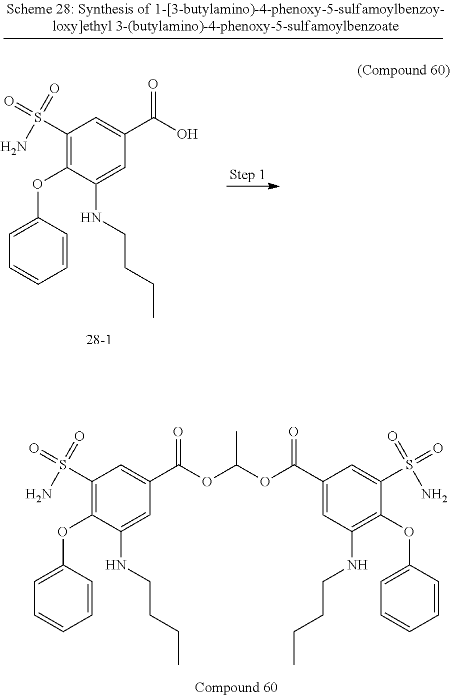 [SMILES: CCCCNc1cc(C(=O)O)cc(S(N)(=O)=O)c1Oc1ccccc1 ; CCCCNc1cc(C(=O)OC(C)OC(=O)c2cc(NCCCC)c(Oc3ccccc3)c(S(N)(=O)=O)c2)cc(S(N)(=O)=O)c1Oc1ccccc1]

Step 1: Preparation of 1-[3-(butylamino)-4-phenoxy-5-sulfamoylbenzoyloxy]ethyl 3-(butylamino)-4-phenoxy-5-sulfamoylbenzoate (Compound 60): To a solution of bumetanide 28-1 (3.87 g, 10.64 mmol) in N,N-Dimethylformamide (20 mL) were added K₂CO₃ (1.469 g, 10.64 mmol), TBAI (0.785, 2.12 mmol) and 1,1-dibromoethane (1.0 g, 5.32 mmol) at 0° C. The reaction mixture was allowed to stir at 100° C. for 16 h. The resulting reaction mixture was diluted with ethyl acetate (250 mL), washed with water (2×80 mL), dried over sodium sulfate and concentrated under reduced pressure. The crude product obtained upon evaporation of volatiles was purified through reverse phase column chromatography to obtain Compound 60 as a pale brown solid 430 mg (10%). ¹H NMR (400 MHz, DMSO-d₆) δ 7.71 (d, J=2 Hz, 2H), 7.46-7.39 (m, 6H), 7.30-7.21 (m, 5H), 7.01 (t, J=8 Hz, 2H), 6.84 (d, J=8 Hz, 4H), 5.25 (t, 2H), 3.06 (q, 4H), 1.74 (d, 3H), 1.35 (quintet, 4H), 1.09 (sextet, 4H), 0.76 (t, 6H); MS m/z [M+H]⁺ 755.6.

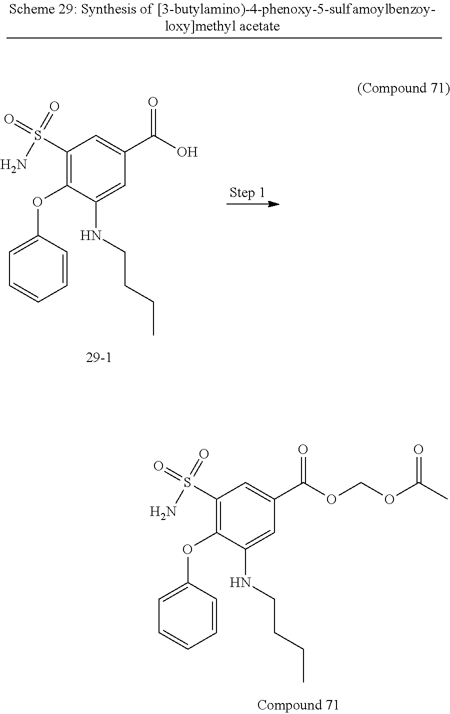 [SMILES: CCCCNc1cc(C(=O)O)cc(S(N)(=O)=O)c1Oc1ccccc1 ; CCCCNc1cc(C(=O)OCOC(C)=O)cc(S(N)(=O)=O)c1Oc1ccccc1]

Step 1: Preparation of [3-(butylamino)-4-phenoxy-5-sulfamoylbenzoyloxy]methyl acetate (Compound 71): To a solution of bumetanide 29-1 (2.1 g, 5.76 mmol) in N,N-Dimethylformamide (15 mL) were added triethylamine (2.08 mL, 14.40 mmol), NaI (1.03 g, 6.9 mmol) and bromomethyl acetate (0.734 mL, 7.49 mmol) drop-wise at 0-5° C. The reaction mixture was allowed to stir at 25-30° C. for 4 h. The resulting reaction mixture was diluted with ethyl acetate (200 mL) and washed with water (2×75 mL), dried over sodium sulfate and concentrated under reduced pressure. The crude product obtained upon evaporation of volatiles was purified through reverse phase column chromatography to obtain product Compound 71 as an off white solid 1.6 g (63%). ¹H NMR (400 MHz, DMSO-d₆) δ 7.69 (d, J=2 Hz, 1H), 7.43-7.39 (m, 3H), 7.29 (t, J=8 Hz, 2H), 7.01 (t, J=8 Hz, 1H), 6.84 (d, J=8 Hz, 2H), 5.94 (s, 2H), 5.25 (t, 1H), 3.06 (q, 2H), 2.12 (s, 3H), 1.36 (quintet, 2H), 1.10 (sextet, 2H), 0.77 (t, 3H); MS m/z [M+H]⁺ 437.3.

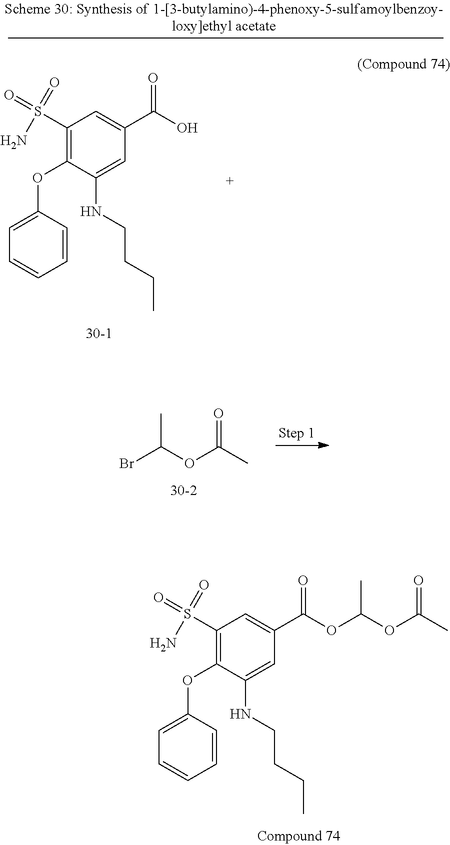

Step 1: Preparation of 1-[3-(butylamino)-4-phenoxy-5-sulfamoylbenzoyloxy]ethyl acetate (Compound 74): To a solution of bumetanide 30-1 (2.4 g, 6.581 mmol) in N,N-Dimethylformamide (15 mL) were added K₂CO₃ (1.18 g, 8.56 mmol), TBAI (0.243, 0.65 mmol) and 1-bromoethyl acetate 30-2 (0.88 mL, 7.90 mmol) at 0° C. The reaction mixture was allowed to stir at 25-30° C. for 2 h. The resulting reaction mixture was diluted with ethyl acetate (250 mL), washed with water (2×80 mL), dried over sodium sulfate and concentrated under reduced pressure. The crude product obtained upon evaporation of volatiles was purified through reverse phase column chromatography to obtain product Compound 74 as pale brown solid 1.45 g (48.9%). ¹H NMR (400 MHz, DMSO-d₆) δ 7.67 (d, J=2 Hz, 1H), 7.45-7.38 (m, 3H), 7.26 (t, J=8 Hz, 2H), 7.05-6.93 (m, 2H), 6.84 (d, J=8 Hz, 2H), 5.21 (t, 1H), 3.06 (q, 2H), 2.09 (s, 3H), 1.56 (d, 3H), 1.36 (quintet, 2H), 1.10 (sextet, 2H), 0.77 (t, 3H); MS m/z [M+H]⁺ 451.4.

### Example 12. Synthesis of Bemetanide Glycolamides

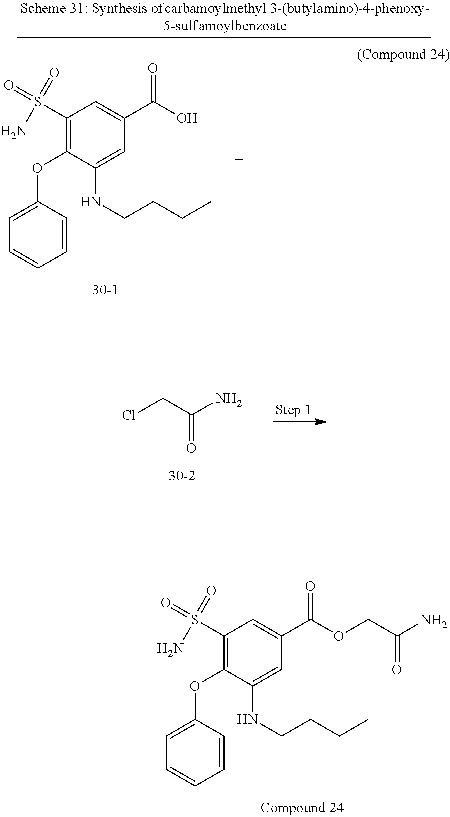 [SMILES: CCCCNc1cc(C(=O)O)cc(S(N)(=O)=O)c1Oc1ccccc1 ; NC(=O)CCl ; CCCCNc1cc(C(=O)OCC(N)=O)cc(S(N)(=O)=O)c1Oc1ccccc1]

Step 1: Preparation of carbamoylmethyl 3-(butylamino)-4-phenoxy-5-sulfamoylbenzoate (Compound 24): To a solution of bumetanide 30-1 (3 g, 8.22 mmol) in N,N-Dimethylformamide (30 mL) were added triethylamine (1.68 mL, 12.3 mmol), NaI (1.35 g, 9.0 mmol) and 2-Chloro-acetamide 30-2 (0.92 g, 9.8 mmol) at 0-5° C. The reaction mixture was allowed to stir at 50° C. over a period of 10 h. The reaction mass was diluted with ethyl acetate (200 mL), washed with water (2×100 mL), dried over sodium sulfate and concentrated under reduced pressure. The crude product obtained upon evaporation of volatiles was purified by silica gel (230-400 mesh) column chromatography to give product Compound 24 as a white solid 1.4 g (40%). ¹H NMR (400 MHz, DMSO-d₆) δ 7.74 (d, J=2 Hz, 1H), 7.59 (s, 1H), 7.47-7.35 (m, 3H), 7.32-7.23 (m, 3H), 7.01 (t, J=8 Hz, 1H), 6.85 (d, J=8 Hz, 2H), 5.16 (t, 1H), 4.71 (s, 2H), 3.07 (q, 2H), 1.37 (quintet, 2H), 1.11 (sextet, 2H), 0.77 (t, 3H); MS m/z [M+H]⁺ 423.6.

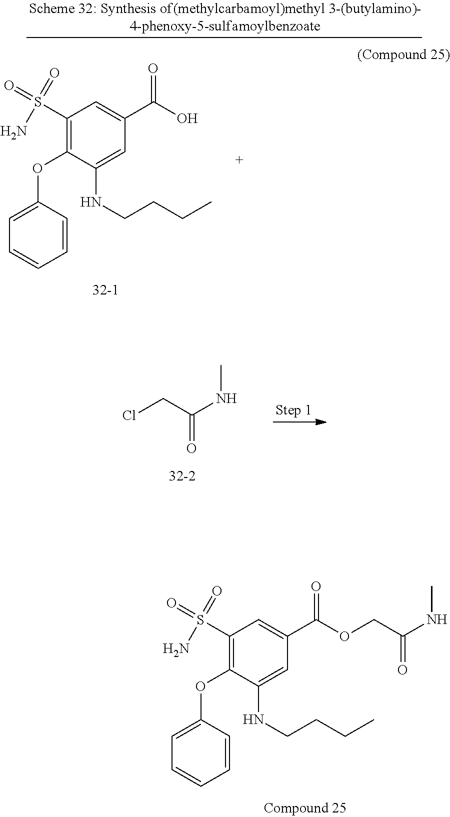

Step 1: Preparation of (methylcarbamoyl)methyl 3-(butylamino)-4-phenoxy-5-sulfamoylbenzoate (Compound 25): To a solution of bumetanide 32-1 (2.5 g, 6.86 mmol) in N,N-Dimethylformamide (25 mL) were added triethylamine (1.4 mL, 10.2 mmol), NaI (1.13 g, 7.5 mmol) and 2-Chloro-N-methyl-acetamide 32-2 (0.88 g, 8.23 mmol) at 0-5° C. The reaction mixture was allowed to stir at 50° C. for 10 h. The reaction mass was diluted with ethyl acetate (300 mL) and washed with water (2×100 mL), dried over sodium sulfate and concentrated under reduced pressure. The crude product obtained upon evaporation of volatiles was purified by silica gel (230-400 mesh) column chromatography to give product Compound 25 as an off white solid 1.8 g (60%). ¹H NMR (400 MHz, DMSO-d₆) δ 8.09 (q, 1H), 7.74 (d, J=2 Hz, 1H), 7.47-7.35 (m, 3H), 7.27 (t, J=8 Hz, 2H), 7.02 (t, J=8 Hz, 1H), 6.85 (d, J=8 Hz, 2H), 5.18 (t, 1H), 4.73 (s, 2H), 3.12-3.01 (m, 2H), 2.64 (d, 3H), 1.37 (quintet, 2H), 1.11 (sextet, 2H), 0.77 (t, 3H); MS m/z [M+H]⁺ 436.7.

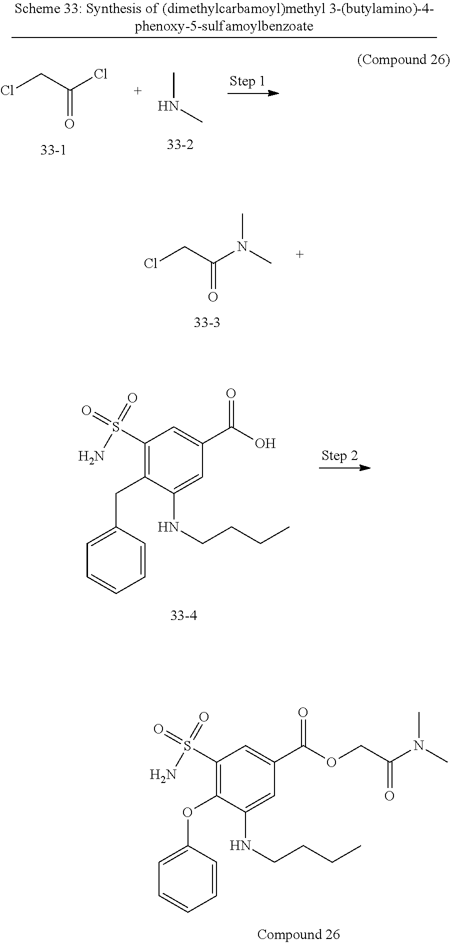

Step 1: Preparation of 2-chloro-N,N-dimethylacetamide (33-3): To a solution of dimethylamine 33-2 (2M in THF, 1.99 g, 44.2 mmol) in dichloromethane (40 mL) was added K₂CO₃ (12.2 g, 88.5 mmol) followed by chloroacetyl chloride 33-1 (3.5 mL, 44.2 mmol) drop-wise at −20° C. The reaction mixture was allowed to stir at same temperature for 1 h. The reaction mass was diluted with ethyl acetate (200 mL), washed with water (2×150 mL), dried over sodium sulfate and concentrated under reduced pressure to give product 33-3 as a pale yellow wax 2.6 g (48%).

Step 2: Preparation of (dimethylcarbamoyl)methyl 3-(butylamino)-4-phenoxy-5-sulfamoylbenzoate (Compound 26): To a solution of bumetanide 33-4 (3 g, 8.2 mmol) in N,N-Dimethylformamide (30 mL) were added triethylamine (1.68 mL, 12.3 mmol), NaI (1.35 g, 9.0 mmol) and 2-chloro-N,N-dimethylacetamide 33-3 (1.2 g, 9.8 mmol) at 0-5° C. The reaction mixture was allowed to stir at 50° C. for 10 h. The resulting reaction mass was diluted with ethyl acetate (200 mL), washed with water (2×100 mL), dried over sodium sulfate and concentrated under reduced pressure. The crude product obtained upon evaporation of volatiles was purified by silica gel (230-400 mesh) column chromatography to give product Compound 26 as a white solid 3.2 g (86%). ¹H NMR (400 MHz, DMSO-d₆) δ 7.74 (d, J=2 Hz, 1H), 7.47-7.35 (m, 3H), 7.27 (t, J=8 Hz, 2H), 7.02 (t, J=8 Hz, 1H), 6.86 (d, J=8 Hz, 2H), 5.18 (t, 1H), 5.07 (s, 2H), 3.13-3.01 (m, 2H), 2.99 (s, 3H), 2.85 (s, 3H), 1.37 (quintet, 2H), 1.11 (sextet, 2H), 0.77 (t, 3H); MS m/z [M+H]⁺ 450.9.

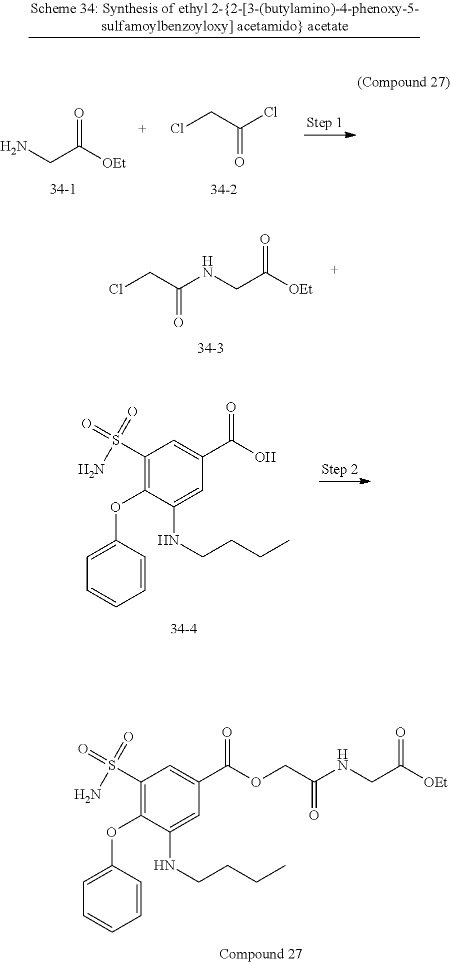 [SMILES: CCOC(=O)CN ; O=C(Cl)CCl ; CCOC(=O)CNC(=O)CCl ; CCCCNc1cc(C(=O)O)cc(S(N)(=O)=O)c1Oc1ccccc1 ; CCCCNc1cc(C(=O)OCC(=O)NCC(=O)OCC)cc(S(N)(=O)=O)c1Oc1ccccc1]

Step 1: Preparation of ethyl (2-chloroacetamido)acetate (34-3): To a solution of ethyl aminoacetate 34-1 (5 g, 48.44 mmol) in dichloromethane (50 mL) were added triethylamine (26.5 mL, 193.9 mmol), 4-Dimethylaminopyridine (0.59 g, 4.8 mmol) followed by chloroacetyl chloride 34-2 (7.82 mL, 96.8 mmol) drop-wise at 0-5° C. The reaction mixture was allowed to stir at 25-30° C. for 4 h. The resulting reaction mass was diluted with ethyl acetate (400 mL), washed with water (2×150 mL), dried over sodium sulfate and concentrated under reduced pressure. The crude product obtained upon evaporation of volatiles was purified by silica gel (230-400 mesh) column chromatography to give product 34-3 as a colourless wax 3.1 g (35%).

Step 2: Preparation of ethyl 2-{2-[3-(butylamino)-4-phenoxy-5-sulfamoylbenzoyloxy]acetamido} acetate (Compound 27): To a solution of bumetanide 34-4 (4 g, 10.9 mmol) in N,N-Dimethylformamide (40 mL) were added K₂CO₃ (1.82 g, 13.7 mmol), TBAI (0.4 g, 1.0 mmol) and ethyl (2-chloroacetamido)acetate 34-3 (2.56 g, 14.2 mmol) at 0-5° C. The reaction mass was allowed to stir at 50° C. for 10 h. The resulting reaction mass was diluted with ethyl acetate (500 mL), washed with water (2×200 mL), dried over sodium sulfate and concentrated under reduced pressure. The crude product obtained upon evaporation of volatiles was purified by silica gel (230-400 mesh) column chromatography to give product Compound 27 as an off white solid 1.6 g (29%). ¹H NMR (400 MHz, DMSO-d₆) δ 8.59 (t, 1H), 7.74 (d, J=2 Hz, 1H), 7.47-7.35 (m, 3H), 7.27 (t, J=8 Hz, 2H), 7.02 (t, J=8 Hz, 1H), 6.85 (d, J=8 Hz, 2H), 5.18 (t, 1H), 4.82 (s, 2H), 4.10 (q, 2H), 3.89 (d, 2H), 3.11-3.02 (m, 2H), 1.37 (quintet, 2H), 1.20 (t, 3H), 1.80 (sextet, 2H), 0.77 (t, 3H); MS m/z [M−H]⁻ 506.8.

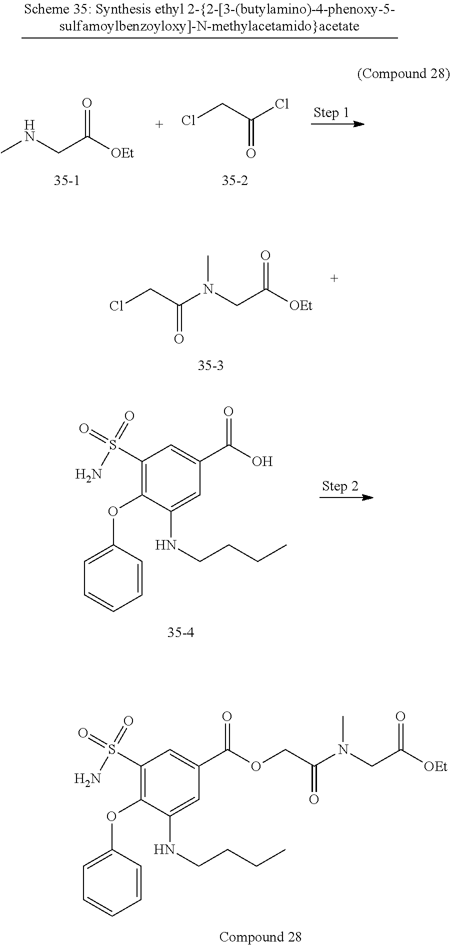

Step 1: Preparation of ethyl [(chloroacetyl)(methyl)amino]acetate (35-3): To a solution of ethyl (methylamino)acetate 35-1 (2.5 g, 21.3 mmol) in dichloromethane (50 mL) were added triethylamine (11.6 mL, 85 mmol), 4-dimethylaminopyridine (0.26 g, 2.13 mmol) and chloroacetyl chloride 35-2 (2.75 mL, 42.6 mmol) drop-wise at 0-5° C. The reaction mass was stirred for 4 h at 25-30° C. The resulting reaction mass was diluted with ethyl acetate (300 mL), washed with water (2×100 mL), dried over sodium sulfate and concentrated under reduced pressure. The crude product obtained upon evaporation of volatiles was purified by silica gel (230-400 mesh) column chromatography to give product 25-3 as a colourless wax 1.9 g (46%).

Step 2: Preparation of ethyl 2-{2-[3-(butylamino)-4-phenoxy-5-sulfamoylbenzoyloxy]-N-methylacetamido}acetate (Compound 28): To a solution of bumetanide 35-4 (2.5 g, 6.8 mmol) in N,N-Dimethylformamide (25 mL) were added K₂CO₃ (1.13 g, 8.2 mmol), TBAI (0.25 g, 0.68 mmol) and ethyl [(chloroacetyl)(methyl)amino]acetate 35-3 (1.46 g, 7.5 mmol) at 0-5° C. The reaction mixture was stirred for 2 h at 25-30° C. The resulting reaction mass was diluted with ethyl acetate (400 mL), washed with water (2×150 mL), dried over sodium sulfate and concentrated under reduced pressure. The crude product obtained upon evaporation of volatiles was purified by silica gel (230-400 mesh) column chromatography to give product Compound 28 as an off white puffy solid 2.4 g (69.9%). ¹H NMR (400 MHz, DMSO-d₆) δ 7.74 (d, J=2 Hz, 1H), 7.46-7.37 (m, 3H), 7.27 (t, J=8 Hz, 2H), 7.01 (t, J=8 Hz, 1H), 6.85 (d, J=8 Hz, 2H), 5.21-5.00 (m, 3H), 4.30-4.06 (m, 4H), 3.10-2.83 (m, 5H), 1.37 (quintet, 2H), 1.20 (t, 3H), 1.10 (sextet, 2H), 0.77 (t, 3H); MS m/z [M−H]⁻ 520.6.

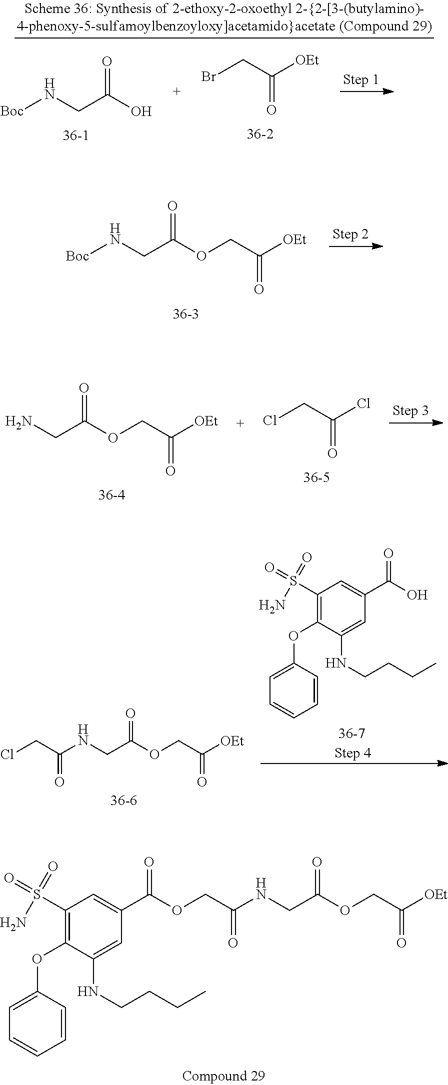

Step 1: Preparation of 2-ethoxy-2-oxoethyl [(tert-butoxycarbonyl)amino]acetate (36-3): To a solution of tert-Butoxycarbonylamino-acetic acid 36-1 (5 g, 28.54 mmol) in N,N-Dimethylformamide (50 mL) were added K₂CO₃ (3.94 g, 28.54 mmol) followed by ethyl bromoacetate 36-2 (4.29 g, 25.68 mmol) drop-wise at 0-5° C. The reaction mass was allowed to stir at 25-30° C. for 2 h. The resulting reaction mass was diluted with ethyl acetate (400 mL), washed with water (2×200 mL), dried over sodium sulfate and concentrated under reduced pressure. The crude product obtained upon evaporation of volatiles was purified by silica gel (230-400 mesh) column chromatography to give product 36-3 as a colourless wax 5.2 g (69%).

Step 2: Preparation of 2-ethoxy-2-oxoethyl aminoacetate (36-4): To a solution of 2-ethoxy-2-oxoethyl [(tert-butoxycarbonyl)amino]acetate 36-3 (5.2 g, 19.9 mmol) in dichloromethane (100 mL) was added TFA (20 mL, 4V) at 0-5° C. The reaction mass was allowed to stir for 1 h at 25-30° C. The reaction progress was monitored by TLC. After completion of reaction, the reaction mass was concentrated under reduced pressure at 45° C. to give 36-4 as a brown wax 8.0 g (crude compound 36-4 was carried as such into next step without any further purification).

Step 3: Preparation of 2-ethoxy-2-oxoethyl (2-chloroacetamido)acetate (36-6): To a solution of 2-ethoxy-2-oxoethyl aminoacetate 36-4 (8.0 g, 49.64 mmol) in dichloromethane (80 mL) was added triethylamine (20.36 mL, 148.9 mmol), 4-Dimethylamino pyridine (0.6 g, 4.96 mmol) and chloroacetyl chloride 36-5 (3.98 mL, 49.64 mmol) drop-wise at 0-5° C. The reaction mass was allowed to stir for 4 h at 25-30° C. The resulting reaction mass was diluted with ethyl acetate (500 mL), washed with water (2×250 mL), dried over sodium sulfate and concentrated under reduced pressure. The crude product obtained upon evaporation of volatiles was purified by silica gel (230-400 mesh) column chromatography to give product 36-6 as a colourless wax 2.7 g (23%).

Step 4: Preparation of 2-ethoxy-2-oxoethyl 2-{2-[3-(butylamino)-4-phenoxy-5-sulfamoylbenzoyloxy]acetamido}acetate (Compound 29): To a solution of bumetanide 36-7 (3.5 g, 9.6 mmol) in N,N-Dimethylformamide (35 mL) were added K₂CO₃ (1.59 g, 11.5 mmol), TBAI (0.35 g, 0.96 mmol) and 2-ethoxy-2-oxoethyl (2-chloroacetamido)acetate 36-6 (3.19 g, 13.44 mmol) at 0-5° C. The reaction mass was stirred at 25-30° C. for 2 h. The resulting reaction mass was diluted with ethyl acetate (300 mL), washed with water (2×150 mL), dried over sodium sulfate and concentrated under reduced pressure. The crude product obtained upon evaporation of volatiles was purified by silica gel (230-400 mesh) column chromatography to give product Compound 29 as an off white solid 1.4 g (25.7%). ¹H NMR (400 MHz, DMSO-d₆) δ 8.69 (t, 1H), 7.74 (d, J=2 Hz, 1H), 7.47-7.35 (m, 3H), 7.27 (t, J=8 Hz, 2H), 7.01 (t, J=8 Hz, 1H), 6.85 (d, J=8 Hz, 2H), 5.18 (t, 1H), 4.83 (s, 2H), 4.72 (s, 2H), 4.14 (q, 2H), 4.02 (d, 2H), 3.11-3.01 (m, 2H), 1.37 (quintet, 2H), 1.20 (t, 3H), 1.10 (sextet, 2H), 0.77 (t, 3H); MS m/z [M+H]⁺ 567.1.

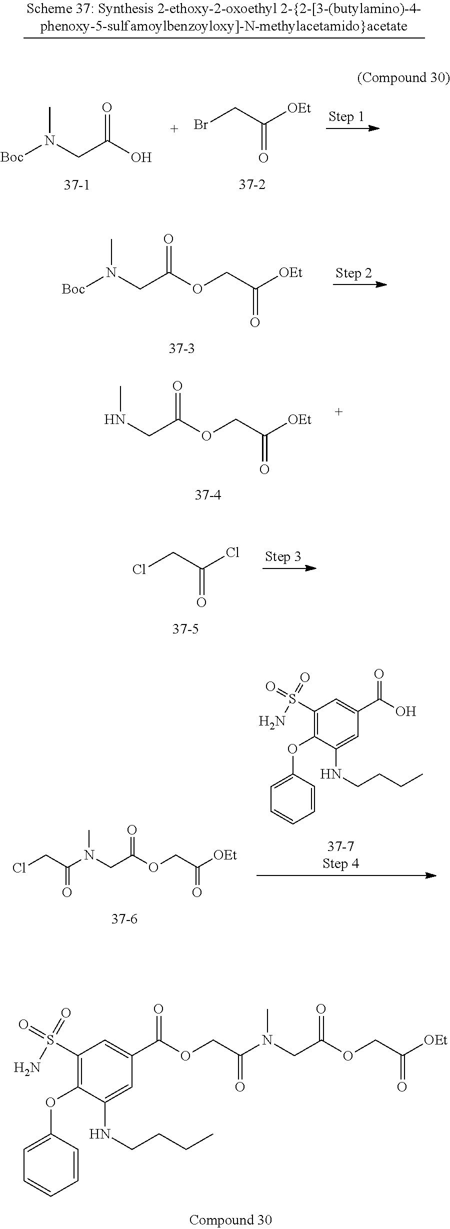

Step 1: Preparation of 2-ethoxy-2-oxoethyl [(tert-butoxycarbonyl)(methyl)amino]acetate (37-3): To a solution of [(tert-butoxycarbonyl)(methyl)amino]acetic acid 37-1 (2.2 g, 11.62 mmol) in N,N-Dimethylformamide (10 mL) were added K₂CO₃ (1.92 g, 13.9 mmol), TBAI (0.42 g, 1.16) followed by ethyl bromoacetate 37-2 (1.55 g, 25.68 mmol) drop-wise at 0-5° C. The reaction mass was allowed to stir at 25-30° C. for 2 h. The resulting reaction mass was diluted with ethyl acetate (200 mL), washed with water (2×100 mL), dried over sodium sulfate and concentrated under reduced pressure. The crude product obtained upon evaporation of volatiles was purified by silica gel (230-400 mesh) column chromatography to give product 37-3 as a colourless wax 2.3 g (71%).

Step 2: Preparation of 2-ethoxy-2-oxoethyl (methylamino)acetate (37-4): To a solution of 2-ethoxy-2-oxoethyl [(tert-butoxycarbonyl)(methyl)amino]acetate 37-3 (2.3 g, 8.35 mmol) in dichloromethane (40 mL), was added TFA (9.2 mL) at 0-5° C. The reaction mass was stirred at 25-30° C. for 1 h. The reaction progress was monitored by TLC. After completion of reaction, the reaction mass was concentrated under reduced pressure at 45° C. to give 37-4 as a brown wax 2.5 g (crude compound 37-4 was carried as such into next step without any purification).

Step 3: Preparation of 2-ethoxy-2-oxoethyl [(chloroacetyl)(methyl)amino]acetate (37-6): To a solution of 2-ethoxy-2-oxoethyl (methylamino)acetate 37-4 (2.5 g, 14.27 mmol) in dichloromethane (50 mL) were added triethylamine (5.85 mL, 42.8 mmol), 4-Dimethylamino pyridine (0.17 g, 1.43 mmol) and chloroacetyl chloride 37-5 (1.12 mL, 14.27 mmol) drop-wise at 0-5° C. The reaction mass was allowed to stir at 25-30° C. for 4 h. The resulting reaction mass was diluted with ethyl acetate (200 mL), washed with water (2×100 mL), dried over sodium sulfate and concentrated under reduced pressure. The crude product obtained upon evaporation of volatiles was purified by silica gel (230-400 mesh) column chromatography to give product 37-6 as a colourless wax 1.3 g (36.2%).

Step 4: Preparation of 2-ethoxy-2-oxoethyl 2-{2-[3-(butylamino)-4-phenoxy-5-sulfamoylbenzoyloxy]-N-methylacetamido}acetate (Compound 30): To a solution of bumetanide 37-7 (2.2 g, 6.0 mmol) in N,N-Dimethylformamide (12 mL) were added triethylamine (2.46 mL, 18.0 mmol), NaI (0.89 g, 6.0 mmol) and 2-ethoxy-2-oxoethyl [(chloroacetyl)(methyl)amino]acetate 37-6 (1.37 g, 5.4 mmol) at 0-5° C. Then reaction mass was stirred at 25-30° C. for 2 h. The resulting reaction mass was diluted with ethyl acetate (250 mL), washed with water (2×100 mL), dried over sodium sulfate and concentrated under reduced pressure. The crude product obtained upon evaporation of volatiles was purified by silica gel (230-400 mesh) column chromatography to give product Compound 30 as an off white solid 1.9 g (54.4%). ¹H NMR (400 MHz, DMSO-d₆) δ 7.74 (d, J=2 Hz, 1H), 7.45-7.37 (m, 3H), 7.27 (t, J=8 Hz, 2H), 7.01 (t, J=8 Hz, 1H), 6.85 (d, J=8 Hz, 2H), 5.21-5.03 (m, 3H), 4.79 & 4.73 (2s, 2H), 4.46 & 4.25 (2s, 2H), 4.19-4.09 (m, 2H), 3.10-2.86 (m, 5H), 1.37 (quintet, 2H), 1.26-1.16 (m, 3H), 1.10 (sextet, 2H), 0.77 (t, 3H); MS m/z [M−H]⁻ 578.9.

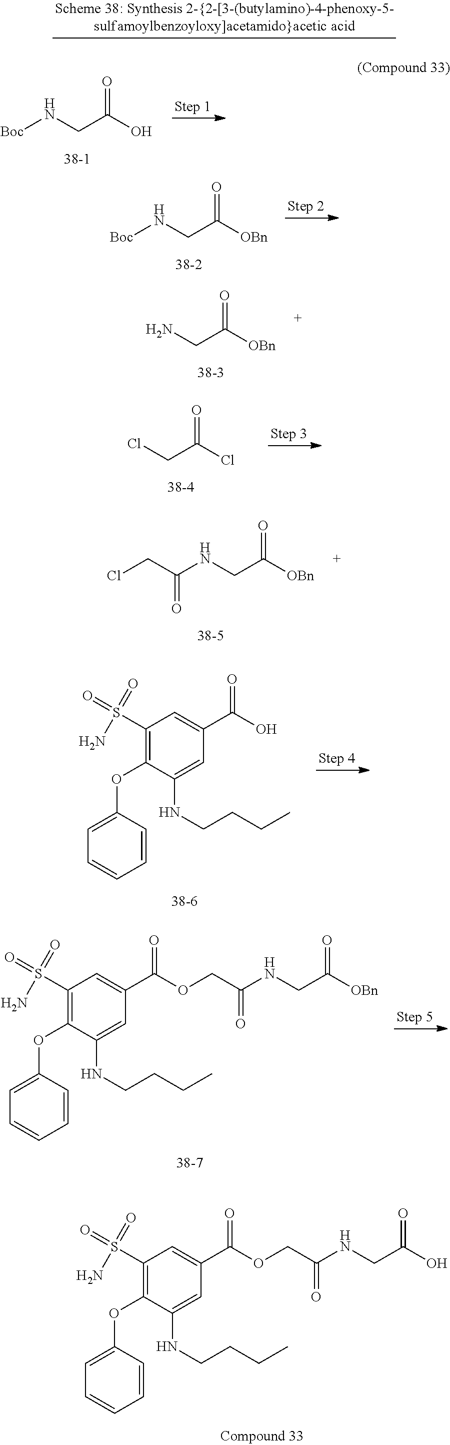

Step 1: Preparation of benzyl [(tert-butoxycarbonyl)amino]acetate (38-2): To a solution of [(tert-butoxycarbonyl) amino]acetic acid 38-1 (35 g, 199.78 mmol) in dichloromethane (350 mL), were added EDC.HCl (57.24 g, 299.6 mmol), benzyl alcohol (17.28 g, 159.82 mmol) and 4-Dimethylaminopyridine (2.43 g, 19.97 mmol) at 0-5° C. The reaction mixture was allowed to stir at 25-30° C. for 1 h. The resulting reaction mass was diluted with ethyl acetate (1.0 L), washed with water (2×500 mL), dried over sodium sulfate and concentrated under reduced pressure. The crude product obtained upon evaporation of volatiles was purified by silica gel (60-120 mesh) column chromatography to give 38-2 as a colourless wax 52 g (98%).

Step 2: Preparation of benzyl aminoacetate (38-3): To a solution of benzyl [(tert-butoxycarbonyl)amino]acetate 38-2 (52 g, 196 mmol) in dichloromethane (500 mL) was added TFA (208 mL, 4V) at 0-5° C. The reaction mass was allowed to stir at 25-30° C. for 1 h. The reaction mass was concentrated under reduced pressure at 45° C. to give 3-3 as a brown wax 70 g (crude compound 38-3 was carried as such into next step without any purification).

Step 3: Preparation of benzyl (2-chloroacetamido)acetate (38-5): To a solution of benzyl aminoacetate 38-3 (70.0 g, 423.8 mmol) in dichloromethane (700 mL) were added triethylamine (173.8 mL, 1271 mmol), 4-Dimethylaminopyridine (5.17 g, 43.38 mmol) and chloroacetyl chloride 38-4 (33.69 mL, 423.8 mmol) drop-wise at 0-5° C. The resulting reaction mixture was allowed to stir at 25-30° C. for 1 h. The resulting reaction mass was diluted with ethyl acetate (1.0 L), washed with water (2×500 mL), dried over sodium sulfate and concentrated under reduced pressure. The crude product obtained upon evaporation of volatiles was purified by silica gel (230-400 mesh) column chromatography to give product 38-5 as a colourless wax 19.35 g (18.8%).

Step 4: Preparation of benzyl 2-{2-[3-(butylamino)-4-phenoxy-5-sulfamoylbenzoyloxy]acetamido}acetate (38-7): To a solution of bumetanide 38-6 (30 g, 82.32 mmol) in N,N-Dimethylformamide (150 mL) were added triethylamine (28.14 mL, 20.58 mmol), NaI (14.8 g, 98.78 mmol) and benzyl (2-chloroacetamido)acetate 38-5 (23.87 g, 98.78 mmol) at 0-5° C. The resulting reaction mixture was allowed to stir at 25-30° C. for 2 h. The resulting reaction mass was diluted with ethyl acetate (1.0 L), washed with water (2×500 mL), dried over sodium sulfate and concentrated under reduced pressure. The crude product obtained upon evaporation of volatiles was purified by silica gel (230-400 mesh) column chromatography to give product 38-7 as an off white solid 19.2 g (40.59%).

Step 5: Preparation of 2-{2-[3-(butylamino)-4-phenoxy-5-sulfamoylbenzoyloxy]acetamido}acetic acid (Compound 33): Pd/C (2.0 g, 20% w/w) was charged to a solution of 2-{2-[3-(butylamino)-4-phenoxy-5-sulfamoylbenzoyloxy]acetamido}acetate 38-7 (10 g, 17.55 mmol) in methanol/dichloromethane (3:7, 40 mL) taken in a Parr-shaker vessel. The reaction mixture was hydrogenated with 5 kg/cm² H₂ pressure at 25-30° C. for 1 h. The reaction progress was monitored by TLC. After completion of the reaction, reaction mass was filtered through celite bed and filtrate was concentrated under reduced pressure to give product Compound 33 as an off white solid 6.0 g (71%). ¹H NMR (400 MHz, DMSO-d₆) δ 12.6 (bs, 1H), 8.50 (t, 1H), 7.74 (d, J=2 Hz, 1H), 7.47-7.35 (m, 3H), 7.27 (t, J=8 Hz, 2H), 7.01 (t, J=8 Hz, 1H), 6.86 (d, J=8 Hz, 2H), 5.18 (t, 1H), 4.82 (s, 2H), 3.82 (d, 2H), 3.11-3.01 (m, 2H), 1.37 (quintet, 2H), 1.80 (sextet, 2H), 0.77 (t, 3H); MS m/z [M−H]⁻ 480.7.

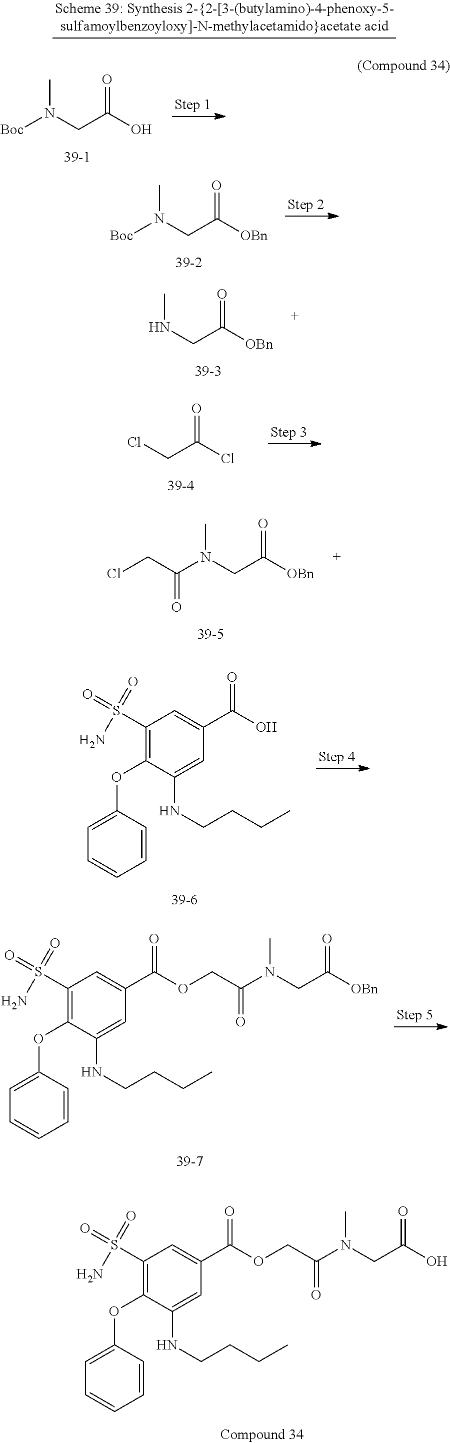

Step 1: Preparation of benzyl [(tert-butoxycarbonyl)(methyl)amino]acetate (39-2): To a solution of [(tert-butoxycarbonyl)(methyl)amino]acetic acid 39-1 (50 g, 0.264 mol) in dichloromethane (200 mL) were added EDC.HCl (75.71 g, 0.396 mol), benzyl alcohol (22.86 g, 0.211 mol) and 4-Dimethylaminopyridine (3.22 g, 0.026 mol) at 0-5° C. The reaction mass was allowed to stir at 25-30° C. for 2 h. The resulting reaction mass was diluted with ethyl acetate (1.0 L), washed with water (2×500 mL), dried over sodium sulfate and concentrated under reduced pressure. The crude product obtained upon evaporation of volatiles was purified by silica gel (60-120 mesh) column chromatography to give product 39-2 as a colourless wax 54.0 g (73%).

Step 2: Preparation of benzyl (methylamino)acetate (39-3): To a solution of benzyl benzyl [(tert-butoxycarbonyl)(methyl)amino]acetate 39-2 (54 g, 0.193 mol) in dichloromethane (200 mL) was added TFA (216 mL, 4V) at 0-5° C. The reaction mass was allowed to stir at 25-30° C. for 1 h. The reaction mass was concentrated under reduced pressure at 45° C. to give 39-3 as a brown wax 85.0 g (crude compound 39-3 was carried as such into next step without any purification).

Step 3: Preparation of benzyl [(chloroacetyl)(methyl)amino]acetate (39-5): To a solution of benzyl (methylamino)acetate 39-3 (85.0 g, 0.474 mol) in dichloromethane (850 mL) were added triethylamine (194.57 mL, 1.422 mol) 4-Dimethylaminopyridine (5.78 g, 0.047 mol) and chloroacetyl chloride 39-4 (56.56 mL, 0.711 mol) drop-wise at 0-5° C. The resulting reaction mixture was allowed to stir at 25-30° C. for 1 h. The resulting reaction mass was diluted with ethyl acetate (1.5 L), washed with water (2×700 mL), dried over sodium sulfate and concentrated under reduced pressure. The crude product obtained upon evaporation of volatiles was purified by silica gel (230-400 mesh) column chromatography to give product 39-5 as a colourless wax 23.0 g (18.9%).

Step 4: Preparation of benzyl 2-{2-[3-(butylamino)-4-phenoxy-5-sulfamoylbenzoyloxy]-N-methylacetamido}acetate (39-7): To a solution of bumetanide 39-6 (19 g, 52.13 mmol) in N,N-Dimethylformamide (100 mL) were added K₂CO₃ (8.64 g, 62.55 mmol), TBAI (1.92 g, 5.21 mmol) and benzyl [(chloroacetyl)(methyl)amino]acetate 39-5 (17.33 g, 67.78 mmol) at 0-5° C. The resulting reaction mixture was allowed to stir at 25-30° C. for 4 h. The resulting reaction mass was diluted with ethyl acetate (1 L), washed with water (2×500 mL), dried over sodium sulfate and concentrated under reduced pressure. The crude product obtained upon evaporation of volatiles was purified by silica gel (230-400 mesh) column chromatography to give 39-7 as an off white solid 21.5 g (69%).

Step 5: Preparation of 2-{2-[3-(butylamino)-4-phenoxy-5-sulfamoylbenzoyloxy]-N-methylacetamido}acetic acid (Compound 34): Pd/C (2.2 g, 20% w/w) was charged to a solution of benzyl 2-{2-[3-(butylamino)-4-phenoxy-5-sulfamoylbenzoyloxy]-N-methylacetamido} acetate 39-7 (21.5 g, 36.83 mmol) in methanol/dichloromethane (3:7.44 mL) taken in a Parr-shaker vessel. The reaction mixture was hydrogenated with 5 kg/cm² H₂ pressure at 25-30° C. for 1 h. The reaction progress was monitored by TLC. After completion of the reaction, reaction mass was filtered through celite bed and filtrate was concentrated under reduced pressure to give product Compound 34 as an off white solid 15.5 g (85.3%). ¹H NMR (400 MHz, DMSO-d₆) δ 12.8 (bs, 1H), 7.74 (d, J=2 Hz, 1H), 7.47-6.99 (m, 3H), 7.27 (t, J=8 Hz, 2H), 7.01 (t, J=8 Hz, 1H), 6.85 (d, J=8 Hz, 2H), 5.22-4.98 (m, 3H), 4.18 & 4.02 (2s, 2H), 3.10-2.81 (m, 5H), 1.37 (quintet, 2H), 1.10 (sextet, 2H), 0.77 (t, 3H); MS m/z [M+H]⁺ 494.9.

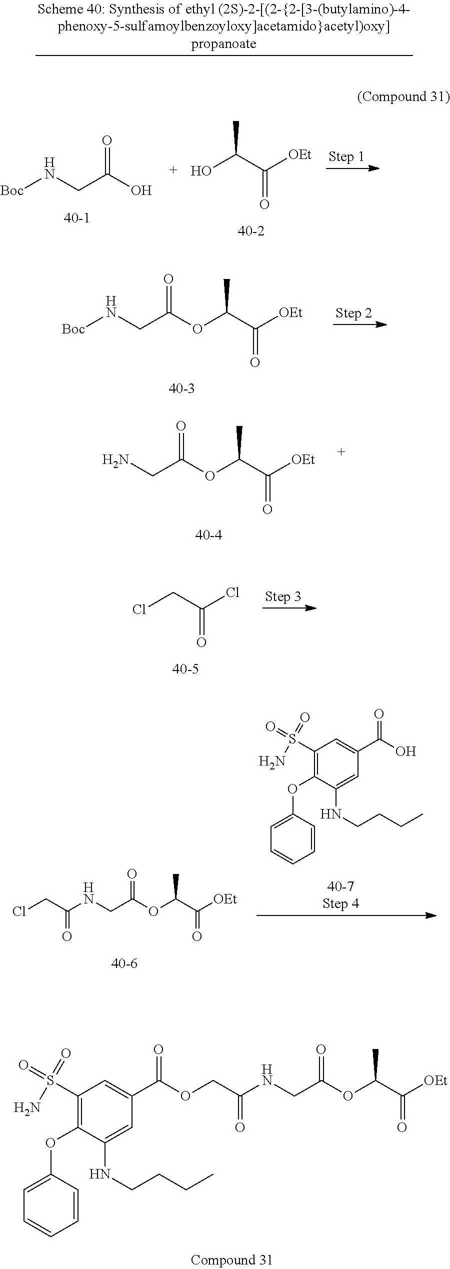

Step 1: Preparation of ethyl (2S)-2-[(2-{[(tert-butoxy)carbonyl]amino}acetyl)oxy]propanoate (40-3): To a solution of [(tert-butoxycarbonyl)amino]acetic acid 40-1 (4.89 g, 27.93 mmol) in dichloromethane (50 mL) were added EDC.HCl (7.27 g, 38.08 mmol), (S)-(−)-ethyl lactate 40-2 (3 g, 25.39 mmol) and 4-Dimethylaminopyridine (0.31 g, 2.59 mmol) at 0-5° C. The reaction mixture was allowed to stir at 25-30° C. for 2 h. The resulting reaction mass was diluted with ethyl acetate (300 mL), washed with water (2×150 mL), dried over sodium sulfate and concentrated under reduced pressure. The crude product obtained upon evaporation of volatiles was purified by silica gel (60-120 mesh) column chromatography to give product 40-3 as a yellow wax 5.5 g (78.6%)

Step 2: Preparation of ethyl (2S)-2-[(2-aminoacetyl)oxy]propanoate (40-4): To a solution of ethyl (2S)-2-[(2-{[(tert-butoxy)carbonyl]amino}acetyl)oxy]propanoate 40-3 (5.5 g, 19.98 mmol) in dichloromethane (100 mL) was added TFA (22 mL, 4V) at 0-5° C. The reaction mixture was allowed to stir at 25-30° C. for 1 h. The progress of reaction was monitored by TLC. After completion of reaction, the reaction mass was concentrated under reduced pressure to give 40-4 as a brown wax 7.0 g (crude compound 40-4 was carried as such into next step without any purification).

Step 3: Preparation of ethyl (2S)-2-{[2-(2-chloroacetamido)acetyl]oxy}propanoate (40-6): To a solution of ethyl (2S)-2-[(2-aminoacetyl)oxy]propanoate 40-4 (7.0 g, 39.96 mmol) in dichloromethane (70 mL) were added triethylamine (16.39 mL, 119.8 mmol) 4-Dimethyl aminopyridine (0.48 g, 3.99 mmol) and chloroacetyl chloride 40-5 (3.18 mL, 39.96 mmol) drop-wise at 0-5° C. The reaction mixture was allowed to stir at 25-30° C. for 1 h. The resulting reaction mass was diluted with ethyl acetate (400 mL), washed with water (2×200 mL), dried over sodium sulfate and concentrated under reduced pressure. The crude product obtained upon evaporation of volatiles was purified by silica gel (230-400 mesh) column chromatography to give product 40-6 as a colourless wax 3.8 g (37.8%).

Step 4: Preparation of ethyl (2S)-2-[(2-{2-[3-(butylamino)-4-phenoxy-5-sulfamoylbenzoyloxy]acetamido}acetyl)oxy]propanoate (Compound 31): To a solution of bumetanide 40-7 (4 g, 10.97 mmol) in N,N-Dimethylformamide (40 mL) were added K₂CO₃ (1.82 g, 13.16 mmol), TBAI (0.405 g, 1.09 mmol) and ethyl (2S)-2-{[2-(2-chloroacetamido)acetyl]oxy}propanoate 40-6 (3.86 g, 15.34 mmol) 0-5° C. The reaction mixture was allowed to stir at 25-30° C. for 2 h. The reaction mass was diluted with ethyl acetate (300 mL) and washed with water (2×150 mL). The organic phase was separated from aqueous, dried over sodium sulfate and concentrated under reduced pressure. The residue obtained was purified by silica gel (230-400 mesh) column chromatography to give product Compound 31 as a white solid 1.0 g (15.7%). ¹H NMR (400 MHz, DMSO-d₆) δ 8.67 (t, 1H), 7.74 (d, J=2 Hz, 1H), 7.48-7.37 (m, 3H), 7.27 (t, J=8 Hz, 2H), 7.02 (t, J=8 Hz, 1H), 6.85 (d, J=8 Hz, 2H), 5.18 (t, 1H), 5.02 (q, 1H), 4.82 (s, 2H), 4.17-4.07 (m, 2H), 4.06-3.88 (m, 2H), 3.11-3.00 (m, 2H), 1.44-1.31 (m, 5H), 1.20 (t, 3H), 1.10 (sextet, 2H), 0.77 (t, 3H); MS m/z [M+H]⁺ 580.9.

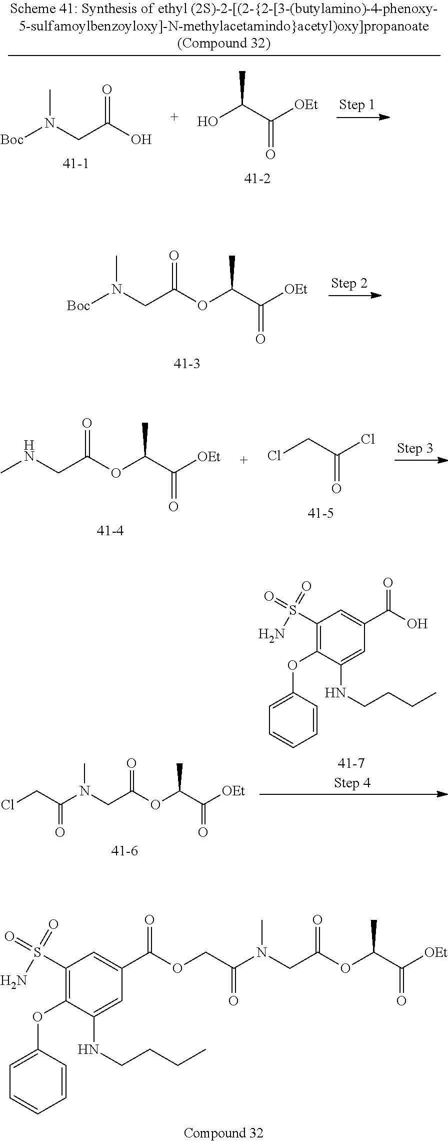

Step 1: Preparation of ethyl (2S)-2-[(2-{[(tert-butoxy)carbonyl](methyl)amino}acetyl)oxy]propanoate (41-3): To a solution of [(tert-butoxycarbonyl)(methyl)amino]acetic acid 41-1 (5.28 g, 27.92 mmol) in dichloromethane (50 mL) were added EDC.HCl (0.724 g, 38.08 mmol), (S)-(−)-ethyl lactate 41-2 (3 g, 25.39 mmol) and 4-Dimethylaminopyridine (0.31 g, 2.5 mmol) at 0-5° C. The reaction mixture was allowed to stir at 25-30° C. for 2 h. The resulting reaction mass was diluted with ethyl acetate (400 mL), washed with water (2×200 mL), dried over sodium sulfate and concentrated under reduced pressure. The crude product obtained upon evaporation of volatiles was purified by silica gel (60-120 mesh) column chromatography to give product 41-3 as a colourless wax 4.1 g (55.7%).

Step 2: Preparation of ethyl (2S)-2-{[2-(methylamino)acetyl]oxy}propanoate (41-4): To a solution of ethyl (2S)-2-[(2-{[(tert-butoxy)carbonyl](methyl)amino}acetyl)oxy] propanoate 41-3 (4.1 g, 14.17 mmol) in dichloromethane (40 mL) was added TFA (16 mL, 4V) at 0-5° C. The reaction mixture was allowed to stir at 25-30° C. for 1 h. The progress of reaction was monitored by TLC. After completion of reaction, the reaction mass was concentrated under reduced pressure to give product 41-4 as a brown wax 6.5 g (crude compound 41-4 was carried as such into next step without any purification).

Step 3: Preparation of ethyl (2S)-2-{[2-(2-chloro-N-methylacetamido)acetyl]oxy}propanoate (41-6): To a solution of ethyl (2S)-2-{[2-(methylamino)acetyl]oxy}propanoate 41-4 (6.5 g, 34.35 mmol) in dichloromethane (65 mL) were added triethylamine (14.09 mL, 103.05 mmol), 4-Dimethylaminopyridine (0.42 g, 3.43 mmol) and chloroacetyl chloride 41-5 (2.73 mL, 34.35 mmol) drop-wise at 0-5° C. The reaction mixture was allowed to stir at 25-30° C. for 1 h. The resulting reaction mass was diluted with ethyl acetate (500 mL), washed with water (2×200 mL), dried over sodium sulfate and concentrated under reduced pressure. The crude product obtained upon evaporation of volatiles was purified by silica gel (230-400 mesh) column chromatography to give product 41-6 as a yellow wax 2.5 g (27.4%).

Step 4: Preparation of ethyl (2S)-2-[(2-{2-[3-(butylamino)-4-phenoxy-5-sulfamoylbenzoyloxy]-N-methylacetamido}acetyl)oxy]propanoate (Compound 32): To a solution of bumetanide 41-7 (2.2 g, 6.04 mmol) in N,N-Dimethylformamide (10 mL) were added K₂CO₃ (1.0 g, 7.25 mmol), TBAI (0.22 g, 0.6 mmol) and ethyl (2S)-2-{[2-(2-chloro-N-methylacetamido)acetyl]oxy}propanoate 41-6 (2.24 g, 8.46 mmol) at 0-5° C. The reaction mixture was allowed to stir at 25-30° C. for 2 h. The reaction mass was diluted with ethyl acetate (300 mL) and washed with water (2×150 mL). The organic phase was separated from aqueous, dried over sodium sulfate and concentrated under reduced pressure. The residue obtained was purified by silica gel (230-400 mesh) column chromatography to give product Compound 32 as a pale yellow solid 2.2 g (61.4%). ¹H NMR (400 MHz, DMSO-d₆) δ 7.74 (d, J=2 Hz, 1H), 7.45-7.38 (m, 3H), 7.27 (t, J=8 Hz, 2H), 7.01 (t, J=8 Hz, 1H), 6.85 (d, J=8 Hz, 2H), 5.22-4.94 (m, 4H), 4.49-4.08 (m, 4H), 3.11-2.84 (m, 5H), 1.47-1.30 (m, 5H), 1.23-1.14 (m, 3H), 1.10 (sextet, 2H), 0.77 (t, 3H); MS m/z [M−H]⁻ 593.0.

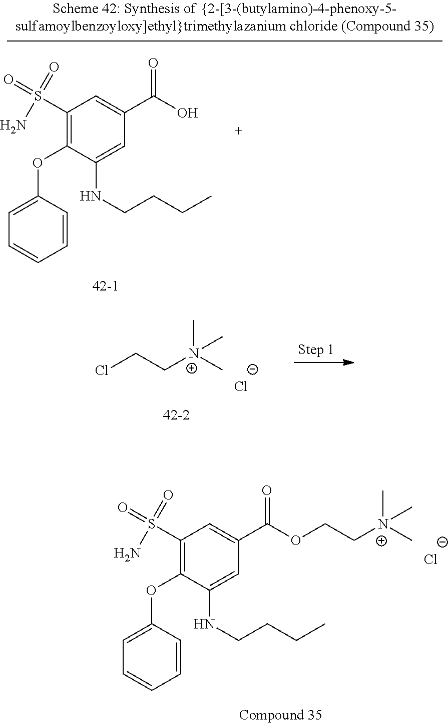

Step 1: Preparation of {2-[3-(butylamino)-4-phenoxy-5-sulfamoylbenzoyloxy]ethyl}trimethylazanium chloride (Compound 35):

To a solution of bumetanide 42-1 (3 g, 8.24 mmol) in N,N-Dimethylformamide (30 mL) were added NaI (1.4 g, 9.890 mmol), triethylamine (1.3 mL, 9.88 mmol) and (2-chloroethyl)trimethylazanium chloride 42-2 (1.5 g, 12.36 mmol) at 25-28° C. The reaction mixture was allowed to stir at 60° C. over a period of 10 h. The resulting reaction mass was cooled to 27° C. and concentrated completely to afford crude compound. The crude compound was purified by preparative HPLC (Agela C18, 330×20 μm; 0-40% acetonitrile/0.05% TFA in water) and lyophilized to obtain product Compound 35 as an off white solid 2.1 g (56.8%). ¹H NMR (400 MHz, DMSO-d₆) δ 7.71 (d, J=2 Hz, 1H), 7.48-7.39 (m, 3H), 7.27 (t, J=8 Hz, 3H), 7.02 (t, J=8 Hz, 1H), 6.83 (d, J=8 Hz, 2H), 5.27 (bs, 1H), 4.77-4.69 (m, 2H), 3.89-3.81 (m, 2H), 3.22 (s, 9H), 3.07 (bt, 2H), 1.35 (quintet, 2H), 1.10 (sextet, 2H), 0.79 (t, 3H); MS m/z [M]⁺450.7.

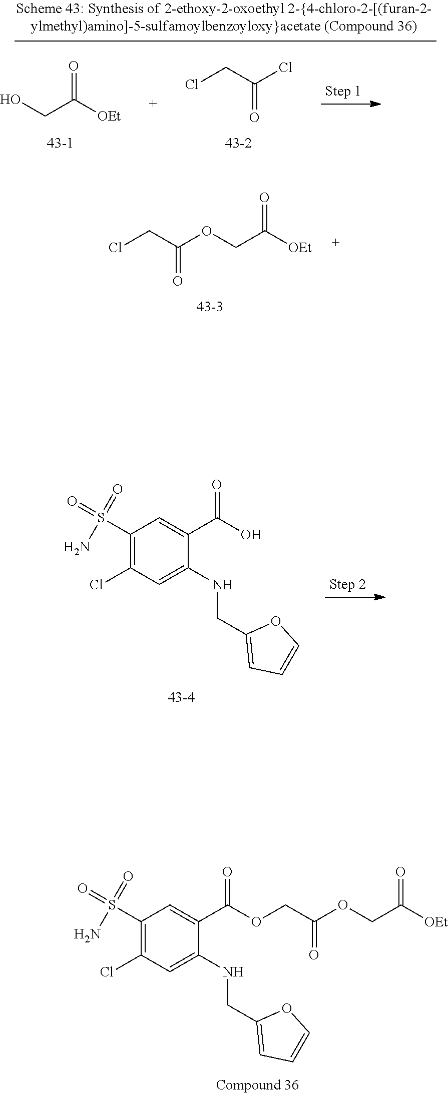 [SMILES: CCOC(=O)CO ; O=C(Cl)CCl ; CCOC(=O)COC(=O)CCl ; NS(=O)(=O)c1cc(C(=O)O)c(NCc2ccco2)cc1Cl ; CCOC(=O)COC(=O)COC(=O)c1cc(S(N)(=O)=O)c(Cl)cc1NCc1ccco1]

Step 1: Preparation of 2-ethoxy-2-oxoethyl chloroacetate (43-3): To a solution of ethyl 2-hydroxyacetate 43-1 (2 g, 19.23 mmol) in dichloromethane (20 mL) was added triethylamine (8 mL, 57.69 mmol), 4-dimethylaminopyridine (0.23 g, 1.92 mmol) and 2-chloroacetyl chloride 43-2 (3.2 mL, 28.84 mmol) at 0-5° C. The reaction mixture was allowed to stir at 25-30° C. over a period of 2 h. The resulting reaction mass was diluted with ethyl acetate (300 mL), washed with water (2×100 mL), dried over sodium sulfate and concentrated under reduced pressure. The crude product obtained upon evaporation of volatiles was purified by silica gel column chromatography to obtain product 43-3 as a colourless wax 2 g (58%).

Step 2: Preparation of 2-ethoxy-2-oxoethyl 2-{4-chloro-2-[(furan-2-ylmethyl)amino]-5-sulfamoylbenzoyloxy}acetate (Compound 36): To a solution of 4-chloro-2-[(furan-2-ylmethyl)amino]-5-sulfamoylbenzoic acid 43-4 (2 g, 6.06 mmol) taken in N,N-Dimethylformamide (6 mL), was added potassium carbonate (1.28 g, 9.09 mmol), and 2-ethoxy-2-oxoethyl 2-chloroacetate 43-3 (1.25 g, 9.09 mmol) at 0° C. The resulting reaction mass was diluted with ethyl acetate (300 mL), washed with water (2×100 mL), dried over sodium sulfate and concentrated under reduced pressure. The crude product obtained upon evaporation of volatiles was purified by silica gel column chromatography to obtain product Compound 36 as a white solid 1.5 g (53%). ¹H NMR (400 MHz, DMSO-d₆) δ 8.48 (s, 1H), 8.36 (t, 1H), 7.62 (d, J=1 Hz, 1H), 7.40 (s, 2H), 7.15 (s, 1H), 6.44-6.34 (m, 2H), 5.06 (s, 2H), 4.78 (s, 2H), 4.62 (d, J=6 Hz, 2H), 4.14 (q, 2H), 1.20 (t, 3H); MS m/z [M−H]⁻ 473.8.

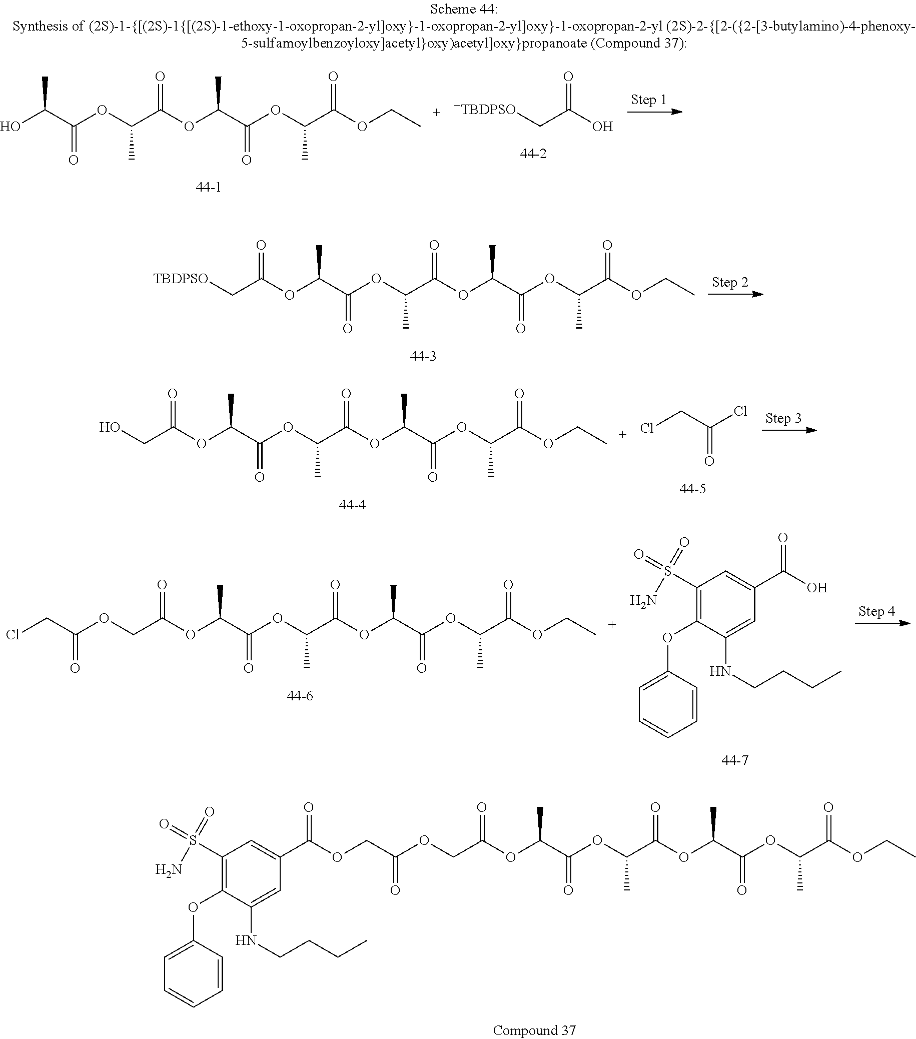

Step 1: Preparation of (2S)-1-{[(2S)-1-{[(2S)-1-ethoxy-1-oxopropan-2-yl]oxy}-1-oxopropan-2-yl]oxy}-1-oxopropan-2-yl (2S)-2-({2-[(tert-butyldiphenylsilyl)oxy]acetyl}oxy)propanoate (44-3): To a solution of 2-[(tert-butyldiphenylsilyl)oxy]acetic acid 44-2 (8 g, 23.9 mmol) in dichloromethane (80 mL) were added EDCI.HCl (6.8 g, 35.9 mmol), (2S)-1-{[(2S)-1-{[(2S)-1-ethoxy-1-oxopropan-2-yl]oxy}-1-oxopropan-2-yl]oxy}-1-oxopropan-2-yl (2S)-2-hydroxypropanoate 44-1 (10.37 g, 31.07 mmol) and 4-Dimethylaminopyridine (0.29 g, 2.39 mmol) at 0° C. The reaction mixture was stirred at 25-30° C. for 1 h. The resulting reaction mass was diluted with ethyl acetate (500 mL), washed with water (2×200 mL), dried over sodium sulfate and concentrated under reduced pressure. The crude product obtained upon evaporation of volatiles was purified by silica gel column chromatography to obtain product 44-3 as a colourless wax 14 g (93%).

Step 2: Preparation of (2S)-1-{[(2S)-1-{[(2S)-1-ethoxy-1-oxopropan-2-yl]oxy}-1-oxopropan-2-yl]oxy}-1-oxopropan-2-yl (2S)-2-[(2-hydroxyacetyl)oxy]propanoate (44-4): To a solution of (2S)-1-{[(2S)-1-{[(2S)-1-ethoxy-1-oxopropan-2-yl]oxy}-1-oxopropan-2-yl]oxy}-1-oxopropan-2-yl (2S)-2-({2-[(tert-butyldiphenylsilyl)oxy]acetyl}oxy)propanoate 44-3 (4 g, 6.34 mmol) in tetrahydrofuran (40 mL) were added acetic acid (0.43 mL, 7.60 mmol) and TBAF (7.2 mL, 7.60 mmol) at 0-5° C. The reaction mixture was stirred at 0-5° C. for 1 h. The resulting reaction mass was diluted with ethyl acetate (200 mL), washed with water (2×100 mL), dried over sodium sulfate and concentrated under reduced pressure. The crude product obtained upon evaporation of volatiles was purified by silica gel column chromatography to obtain product 44-4 as a colourless wax 2.2 g (91%).

Step 3: Preparation of (2S)-1-{[(2S)-1-{[(2S)-1-ethoxy-1-oxopropan-2-yl]oxy}-1-oxopropan-2-yl]oxy}-1-oxopropan-2-yl(2S)-2-({2-[(2-chloroacetyl)oxy]acetyl}oxy)propanoate (44-6):

To a solution of (2S)-1-{[(2S)-1-{[(2S)-1-ethoxy-1-oxopropan-2-yl]oxy}-1-oxopropan-2-yl]oxy}-1-oxopropan-2-yl (2S)-2-[(2-hydroxyacetyl)oxy]propanoate 44-4 (2 g, 5.10 mmol) in dichloromethane (20 mL) were added triethylamine (2.1 mL, 15.30 mmol), chloroacetyl chloride 44-5 (0.40 mL, 7.65 mmol) and 4-Dimethylaminopyridine (0.06 g, 0.51 mmol) at 0-5° C. The reaction mixture was stirred at 25-30° C. for 2 h. The resulting reaction mass was diluted with ethyl acetate (200 mL), washed with water (2×100 mL), dried over sodium sulfate and concentrated under reduced pressure. The crude product obtained upon evaporation of volatiles was purified by silica gel column chromatography to obtain product 44-6 as a colourless wax 2.0 g (86%).

Step 4: Preparation of (2S)-1-{[(2S)-1-{[(2S)-1-ethoxy-1-oxopropan-2-yl]oxy}-1-oxopropan-2-yl]oxy}-1-oxopropan-2-yl (2S)-2-{[2-({2-[3-(butylamino)-4-phenoxy-5-sulfamoylbenzoyloxy]acetyl}oxy)acetyl]oxy}propanoate (Compound 37):

To a solution of 3-(butylamino)-4-phenoxy-5-sulfamoylbenzoic acid 44-7 (1.2 g, 3.29 mmol) in N,N-Dimethylformamide (3.6 mL) were added potassium carbonate (0.59 g, 4.27 mmol), TBAI (0.12 g, 0.329 mmol) and (2S)-1-{[(2S)-1-{[(2S)-1-ethoxy-1-oxopropan-2-yl]oxy}-1-oxopropan-2-yl]oxy}-1-oxopropan-2-yl(2S)-2-({2-[(2-chloroacetyl)oxy]acetyl}oxy) propanoate 44-6 (2 g, 4.27 mmol) at 0-5° C. The reaction mixture was allowed to stir at 25-30° C. over a period of 2 h. The resulting reaction mass was diluted with ethyl acetate (200 mL), washed with water (2×100 mL), dried over sodium sulfate and concentrated under reduced pressure. The crude product obtained upon evaporation of volatiles was purified by silica gel column chromatography to obtain product 33-6 as an off white low melting solid 1.3 g (50%). ¹H NMR (400 MHz, DMSO-d₆) δ 7.74 (d, J=2 Hz 1H), 7.46-7.39 (m, 3H), 7.27 (t, 2H), 7.02 (t, 1H), 6.85 (d, 2H), 5.27-5.15 (m, 4H), 5.13-5.04 (m, 3H), 4.95-4.84 (m, 2H), 4.17-4.06 (m, 2H), 3.10-3.01 (m, 2H), 1.50-1.20 (m, 14H), 1.18 (t, 3H), 1.10 (sextet, 2H), 0.77 (t, 3H); MS m/z [M+H]⁺ 797.7.

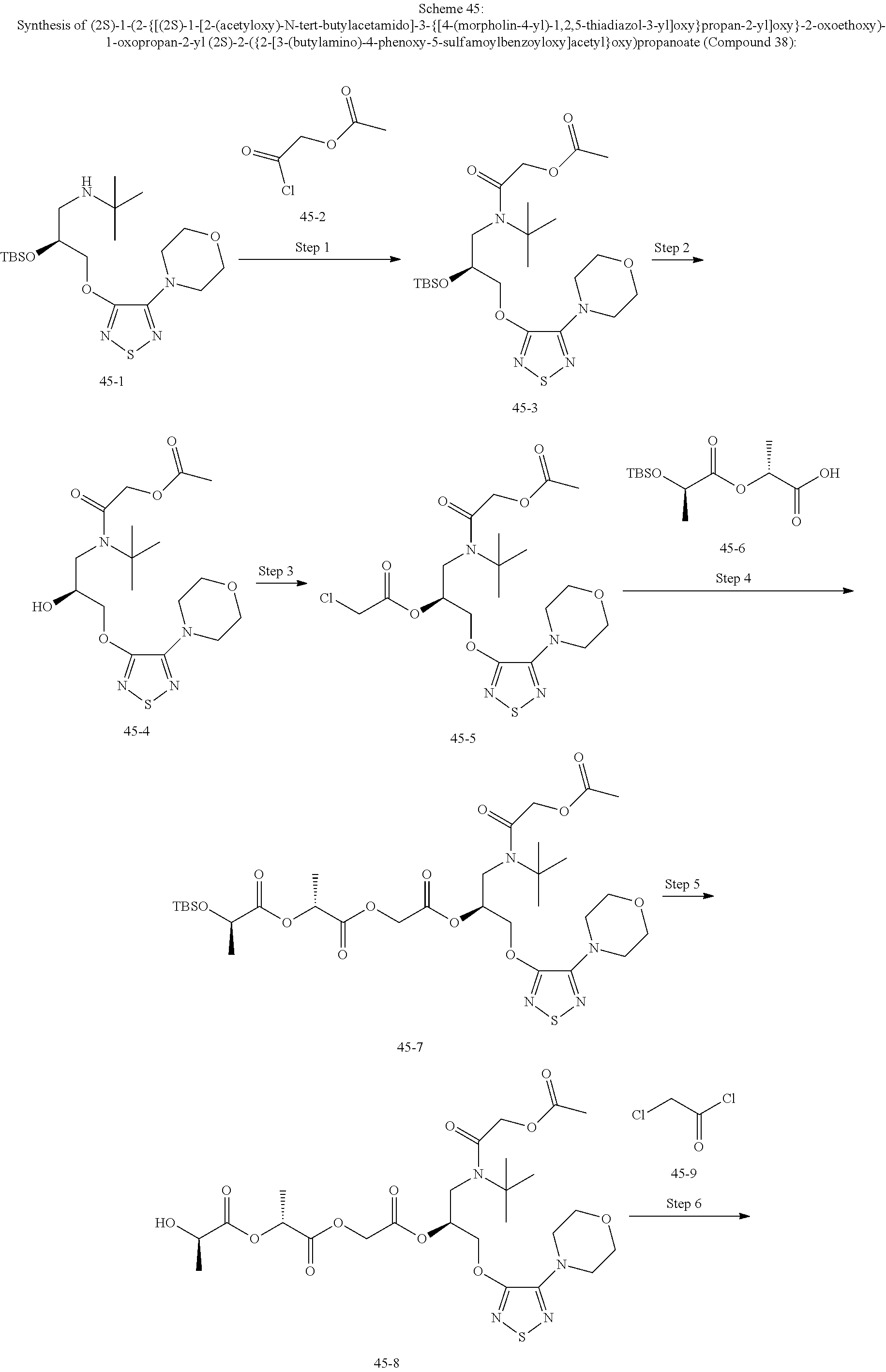

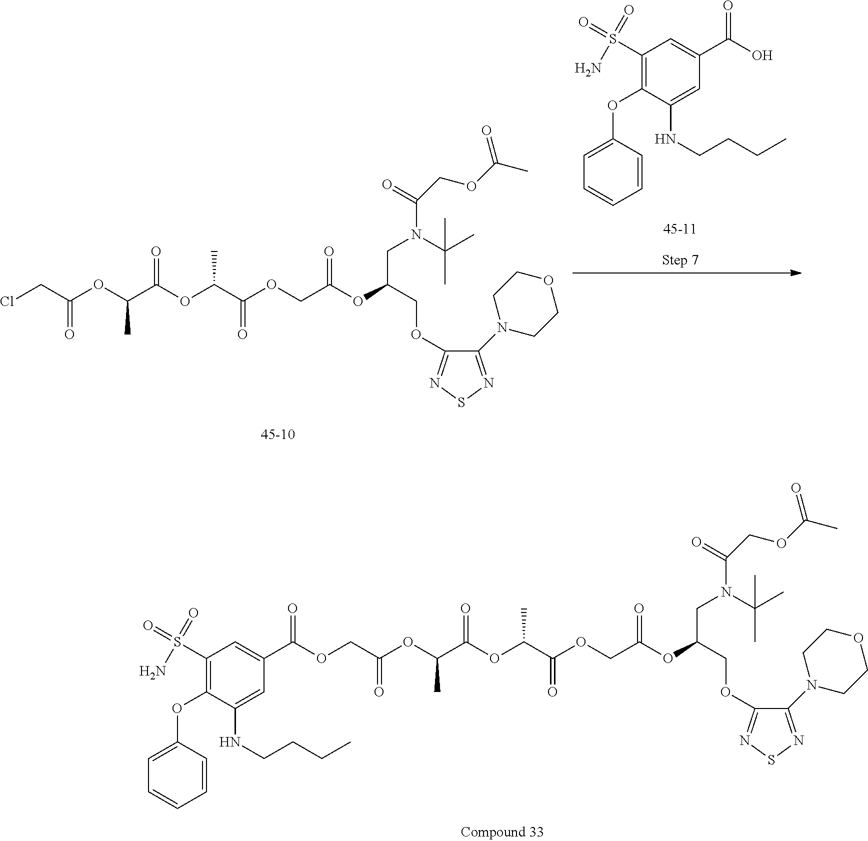 [SMILES: CC(=O)OCC(=O)N(C[C@@H](COc1nsnc1N1CCOCC1)OC(=O)COC(=O)[C@@H](C)OC(=O)[C@@H](C)OC(=O)CCl)C(C)(C)C ; CCCCNc1cc(C(=O)O)cc(S(N)(=O)=O)c1Oc1ccccc1 ; CCCCNc1cc(C(=O)OCC(=O)O[C@H](C)C(=O)O[C@H](C)C(=O)OCC(=O)O[C@H](COc2nsnc2N2CCOCC2)CN(C(=O)COC(C)=O)C(C)(C)C)cc(S(N)(=O)=O)c1Oc1ccccc1]

Step 1: Preparation of {tert-butyl[(2S)-2-[(tert-butyldimethylsilyl)oxy]-3-{[4-(morpholin-4-yl)-1,2,5-thiadiazol-3-yl]oxy}propyl]carbamoyl}methyl acetate (45-3): To a solution of tert-butyl[(2S)-2-[(tert-butyldimethylsilyl)oxy]-3-{[4-(morpholin-4-yl)-1,2,5-thiadiazol-3-yl]oxy}propyl]amine 45-1 (21 g, 48.83 mmol) in chloroform (210 mL) were added triethylamine (13.72 ml, 97.66 mmol) and acetoxyacetyl chloride 45-2 (8.3 mL, 73.25 mmol) at 0-5° C. The reaction mixture was stirred at 25-30° C. over a period of 2 h. The resulting reaction mass was diluted with ethyl acetate (1.0 L), washed with water (2×500 mL), dried over sodium sulfate and concentrated under reduced pressure. The crude product obtained upon evaporation of volatiles was purified by silica gel column chromatography to obtain product 45-3 as a colourless liquid 24 g (93%).

Step 2: Preparation of {tert-butyl[(2S)-2-hydroxy-3-{[4-(morpholin-4-yl)-1,2,5-thiadiazol-3-yl]oxy}propyl]carbamoyl}methyl acetate (45-4): To a solution of {tert-butyl[(2S)-2-[(tert-butyldimethylsilyl)oxy]-3-{[4-(morpholin-4-yl)-1,2,5-thiadiazol-3-yl]oxy}propyl]carbamoyl}methyl acetate 45-3 (24 g, 45.22 mmol) in tetrahydrofuran (240 mL) was added tetra butyl ammonium fluoride (67.83 mL, 1.0 M, 67.83 mmol) at 0-5° C. The reaction mixture was allowed to stir at room temperature over a period of 1 h. The resulting reaction mixture was concentrated under reduced pressure. The crude product obtained upon evaporation of the volatiles was purified through silica gel (60-120 mesh) column chromatography to give product 45-4 as a colourless liquid 12.5 g (66.4%).

Step 3: Preparation of (2S)-1-[2-(acetyloxy)-N-tert-butylacetamido]-3-{[4-(morpholin-4-yl)-1,2,5-thiadiazol-3-yl]oxy}propan-2-yl2-chloroacetate (45-5): To a solution of {tert-butyl[(2S)-2-hydroxy-3-{[4-(morpholin-4-yl)-1,2,5-thiadiazol-3-yl]oxy}propyl]carbamoyl}methyl acetate 45-4 (9 g, 21.61 mmol) in dichloromethane (90 mL) were added triethylamine (15.18 ml, 108.05 mmol), chloroacetyl chloride (4.36 mL, 54.03 mmol) and 4-Dimethylaminopyridine (0.26 g 2.161 mmol) at 0° C. The reaction mixture was stirred at 25-30° C. over a period of 4 h. The resulting reaction mixture was quenched with water (200 mL), extracted with dichloromethane (2×300 mL) and dried over sodium sulfate. The volatiles were evaporated under reduced pressure to obtain product 45-5 as a colourless liquid 4.4 g (41.5%).

Step 4: Preparation of (2S)-1-(2-{[(2S)-1-[2-(acetyloxy)-N-tert-butylacetamido]-3-{[4-(morpholin-4-yl)-1,2,5-thiadiazol-3-yl]oxy}propan-2-yl]oxy}-2-oxoethoxy)-1-oxopropan-2-yl (2S)-2-[(tert-butyldimethylsilyl)oxy]propanoate (45-7): To a solution of 2S)-1-[2-(acetyloxy)-N-tert-butylacetamido]-3-{[4-(morpholin-4-yl)-1,2,5-thiadiazol-3-yl]oxy}propan-2-yl 2-chloroacetate 45-5 (4.5 g, 9.14 mmol) in N,N-Dimethylformamide (13.5 mL) were added Potassium carbonate (1.6 g, 11.89 mmol), TBAI (0.33 g, 0.914 mmol) and 2-({2-[(tert-butyldimethylsilyl)oxy]propanoyl}oxy)propanoic acid 45-6 (3.2 g, 11.88 mmol) at 0° C. The reaction mixture was allowed to stir at 25-30° C. over a period of 2 h. The resulting reaction mass was diluted with ethyl acetate (500 mL), washed with water (2×200 mL), dried over sodium sulfate and concentrated under reduced pressure. The crude product obtained upon evaporation of volatiles was purified through column chromatography to obtain product 45-7 as a colourless wax 4.1 g (61%).

Step 5: Preparation of (2S)-1-(2-{[(2S)-1-[2-(acetyloxy)-N-tert-butylacetamido]-3-{[4-(morpholin-4-yl)-1,2,5-thiadiazol-3-yl]oxy}propan-2-yl]oxy}-2-oxoethoxy)-1-oxopropan-2-yl (2S)-2-hydroxypropanoate (45-8): To a solution of (2S)-1-(2-{[(2S)-1-[2-(acetyloxy)-N-tert-butylacetamido]-3-{[4-(morpholin-4-yl)-1,2,5-thiadiazol-3-yl]oxy}propan-2-yl]oxy}-2-oxoethoxy)-1-oxopropan-2-yl (2S)-2-[(tert-butyldimethylsilyl)oxy]propanoate 45-7 (4.1 g, 5.60 mmol) in 1,4 dioxane (30 mL) was added 1N HCl (5.6 mL, 5.60 mmol) at 0° C. The reaction mixture was allowed to stir at 25-30° C. over a period of 2 h. The resulting reaction mass was diluted with ethyl acetate (500 mL), washed with water (2×200 mL), dried over sodium sulfate and concentrated under reduced pressure. The crude product obtained upon evaporation of volatiles was purified by silica gel column chromatography to obtain product 45-8 as a colourless wax 2.2 g (64%).

Step 6: Preparation (2S)-1-(2-{[(2S)-1-[2-(acetyloxy)-N-tert-butylacetamido]-3-{[4-(morpholin-4-yl)-1,2,5-thiadiazol-3-yl]oxy}propan-2-yl]oxy}-2-oxoethoxy)-1-oxopropan-2-yl (2S)-2-[(2-chloroacetyl)oxy]propanoate (45-10): To a solution of (2S)-1-(2-{[(2S)-1-[2-(acetyloxy)-N-tert-butylacetamido]-3-{[4-(morpholin-4-yl)-1,2,5-thiadiazol-3-yl]oxy}propan-2-yl]oxy}-2-oxoethoxy)-1-oxopropan-2-yl (2S)-2-hydroxypropanoate 45-8 (2.2 g, 3.23 mmol) in dichloromethane (40 mL) were added chloroacetyl chloride 45-9 (0.66 mL, 8.09 mmol) and 4-Dimethylaminopyridine (0.039 g, 0.323 mmol) at 0° C. The reaction mass was stirred at 25-30° C. for 4 h. The reaction mass was diluted with ethyl acetate (400 mL), washed with water (100 mL), dried over sodium sulfate and concentrated under reduced pressure. The crude product obtained upon evaporation of volatiles was purified by silica gel column chromatography to obtain product 45-10 as a colourless wax 2 g (91%).

Step 7: Preparation of (2S)-1-(2-{[(2S)-1-[2-(acetyloxy)-N-tert-butylacetamido]-3-{[4-(morpholin-4-yl)-1,2,5-thiadiazol-3-yl]oxy}propan-2-yl]oxy}-2-oxoethoxy)-1-oxopropan-2-yl (2S)-2-({2-[3-(butylamino)-4-phenoxy-5-sulfamoylbenzoyloxy]acetyl}oxy) propanoate (Compound 33): To a solution of 3-(butylamino)-4-phenoxy-5-sulfamoylbenzoic acid 45-11 (0.9 g, 2.47 mmol) in N,N-Dimethylformamide (2.7 mL) were added potassium carbonate (0.4 g, 2.96 mmol), TBAI (0.091 g, 0.247 mmol) and (2S)-1-(2-{[(2S)-1-[2-(acetyloxy)-N-tert-butylacetamido]-3-{[4-(morpholin-4-yl)-1,2,5-thiadiazol-3-yl]oxy}propan-2-yl]oxy}-2-oxoethoxy)-1-oxopropan-2-yl (2S)-2-[(2-chloroacetyl)oxy]propanoate 45-10 (1.8 g, 2.71 mmol) at 0-5° C. The reaction mixture was allowed to stir at 25-30° C. over a period of 2 h. The resulting reaction mass was diluted with ethyl acetate (300 mL), washed with water (100 mL), dried over sodium sulfate and concentrated under reduced pressure. The crude product obtained upon evaporation of volatiles was purified by silica gel column chromatography to obtain product Compound 33 as a colourless wax 1.4 g (56%). ¹H-NMR (400 MHz, DMSO-d6) δ 7.73 (d, J=2 Hz, 1H), 7.45-7.37 (m, 3H), 7.27 (t, J=8 Hz, 2H), 7.01 (t, J=8 Hz, 1H), 6.85 (d, J=8 Hz, 2H), 5.52-5.42 (m, 1H), 5.30-5.19 (m, 3H), 5.12-5.00 (m, 2H), 4.96-4.77 (m, 3H), 4.70-4.55 (m, 2H), 4.50-4.40 (m, 1H), 3.71-3.53 (m, 6H), 3.44-3.3 (m, 4H), 3.06 (q, 2H), 2.08 (s, 3H), 1.51-1.43 (m, 6H), 1.41-1.25 (m, 11H), 1.15-1.02 (m, 2H), 0.76 (t, 3H). MS m/z [M+H]⁺ 1025.0.

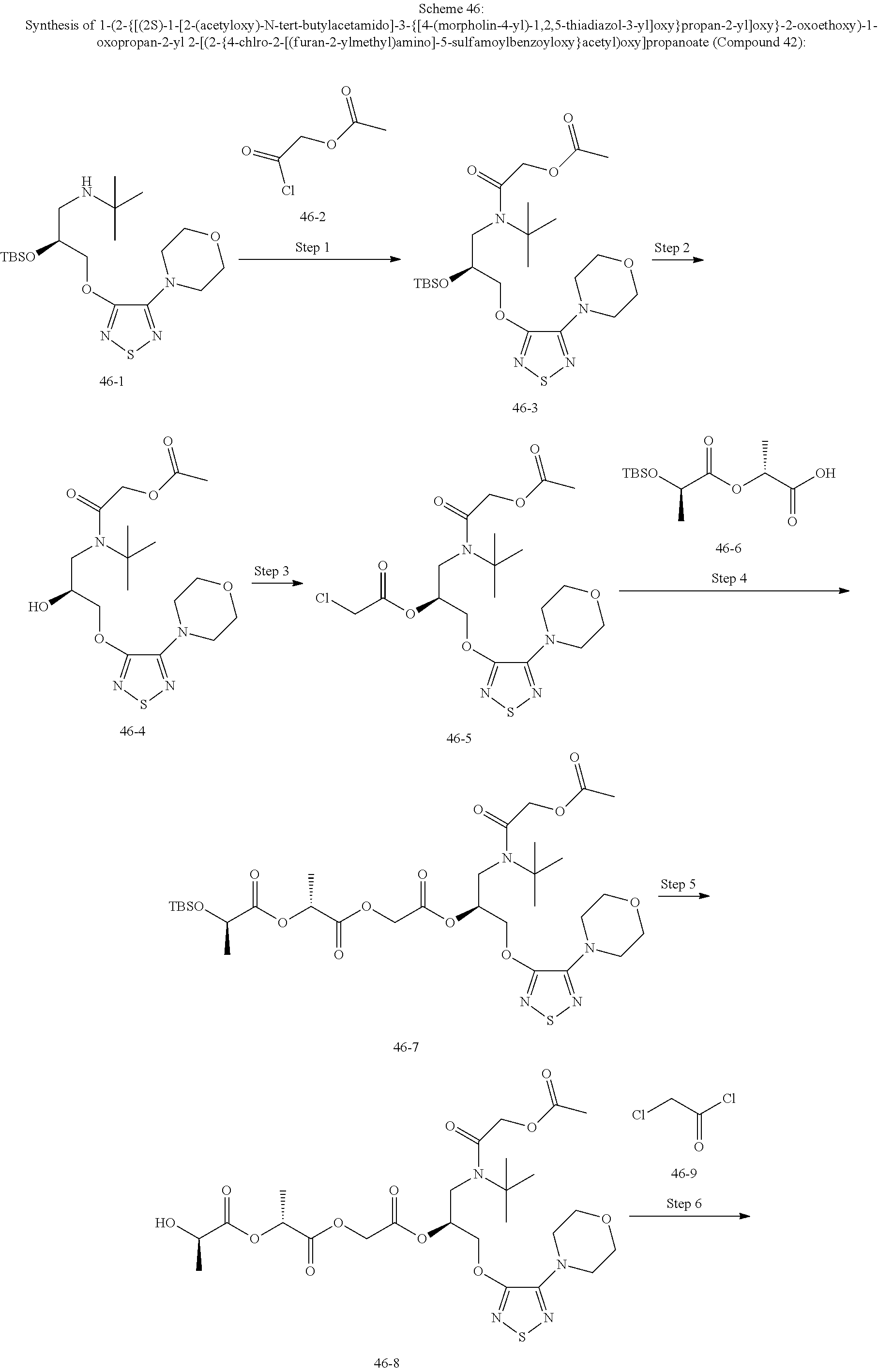

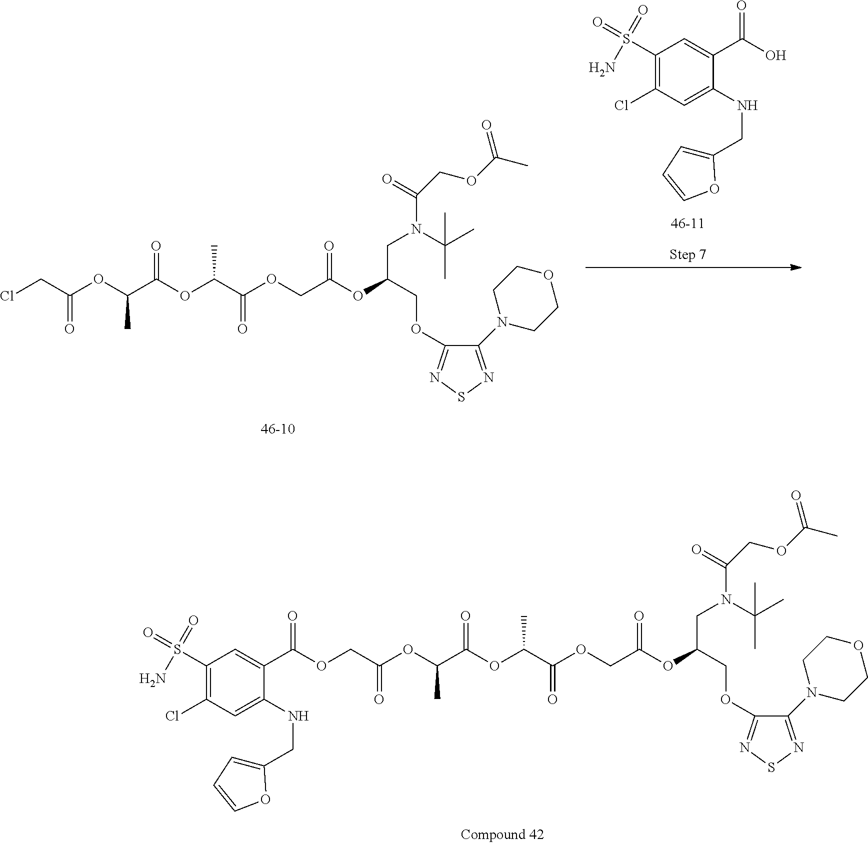 [SMILES: CC(=O)OCC(=O)N(C[C@@H](COc1nsnc1N1CCOCC1)OC(=O)COC(=O)[C@@H](C)OC(=O)[C@@H](C)OC(=O)CCl)C(C)(C)C ; NS(=O)(=O)c1cc(C(=O)O)c(NCc2ccco2)cc1Cl ; CC(=O)OCC(=O)N(C[C@@H](COc1nsnc1N1CCOCC1)OC(=O)COC(=O)[C@@H](C)OC(=O)[C@@H](C)OC(=O)COC(=O)c1cc(S(N)(=O)=O)c(Cl)cc1NCc1ccco1)C(C)(C)C]

Step 1: Preparation of {tert-butyl[(2S)-2-[(tert-butyldimethylsilyl)oxy]-3-{[4-(morpholin-4-yl)-1,2,5-thiadiazol-3-yl]oxy}propyl]carbamoyl}methyl acetate (46-3): To a solution of tert-butyl[(2S)-2-[(tert-butyldimethylsilyl)oxy]-3-{[4-(morpholin-4-yl)-1,2,5-thiadiazol-3-yl]oxy}propyl]amine 46-1 (21 g, 48.83 mmol) in chloroform (210 mL) were added triethylamine (13.72 ml, 97.66 mmol) and acetoxyacetyl chloride 46-2 (8.3 mL, 73.25 mmol) at 0-5° C. The reaction mixture was stirred at 25-30° C. over a period of 2 h. The resulting reaction mass was diluted with ethyl acetate (1.0 L), washed with water (2×500 mL), dried over sodium sulfate and concentrated under reduced pressure. The crude product obtained upon evaporation of volatiles was purified by silica gel column chromatography to obtain product 46-3 as a colourless liquid 24 g (93%).

Step 2: Preparation of {tert-butyl[(2S)-2-hydroxy-3-{[4-(morpholin-4-yl)-1,2,5-thiadiazol-3-yl]oxy}propyl]carbamoyl}methyl acetate (46-4): To a solution of {tert-butyl[(2S)-2-[(tert-butyldimethylsilyl)oxy]-3-{[4-(morpholin-4-yl)-1,2,5-thiadiazol-3-yl]oxy}propyl]carbamoyl}methyl acetate 46-3 (24 g, 45.22 mmol) in tetrahydrofuran (240 mL) was added tetra butyl ammonium fluoride (67.83 mL, 1.0 M, 67.83 mmol) at 0-5° C. The reaction mixture was allowed to stir at room temperature over a period of 1 h. The resulting reaction mixture was concentrated under reduced pressure. The crude product obtained upon evaporation of the volatiles was purified through silica gel (60-120 mesh) column chromatography to give product 46-4 as a colourless liquid 12.5 g (66.4%).

Step 3: Preparation of (2S)-1-[2-(acetyloxy)-N-tert-butylacetamido]-3-{[4-(morpholin-4-yl)-1,2,5-thiadiazol-3-yl]oxy}propan-2-yl2-chloroacetate (46-5): To a solution of {tert-butyl[(2S)-2-hydroxy-3-{[4-(morpholin-4-yl)-1,2,5-thiadiazol-3-yl]oxy}propyl]carbamoyl}methyl acetate 46-4 (9 g, 21.61 mmol) in dichloromethane (90 mL) were added triethylamine (15.18 ml, 108.05 mmol), chloroacetyl chloride (4.36 mL, 54.03 mmol) and 4-Dimethylaminopyridine (0.26 g 2.161 mmol) at 0-5° C. The reaction mixture was stirred at 25-30° C. over a period of 4 h. The resulting reaction mixture was quenched with water (500 mL), extracted with dichloromethane (2×200 mL) and dried over sodium sulfate. Then volatiles were evaporated under reduced pressure to obtain product 46-5 as a colourless liquid 4.4 g (41.5%).

Step 4: Preparation of (2S)-1-(2-{[(2S)-1-[2-(acetyloxy)-N-tert-butylacetamido]-3-{[4-(morpholin-4-yl)-1,2,5-thiadiazol-3-yl]oxy}propan-2-yl]oxy}-2-oxoethoxy)-1-oxopropan-2-yl (2S)-2-[(tert-butyldimethylsilyl)oxy]propanoate (46-7): To a solution of 2S)-1-[2-(acetyloxy)-N-tert-butylacetamido]-3-{[4-(morpholin-4-yl)-1,2,5-thiadiazol-3-yl]oxy}propan-2-yl 2-chloroacetate 46-5 (4.5 g, 9.14 mmol) in N,N-Dimethylformamide (13.5 mL) were added potassium carbonate (1.6 g, 11.89 mmol), TBAI (0.33 g, 0.914 mmol) and 2-({2-[(tert-butyldimethylsilyl)oxy]propanoyl}oxy)propanoic acid 46-6 (3.2 g, 11.88 mmol) at 0-5° C. The reaction mixture was allowed to stir at 25-30° C. over a period of 2 h. The resulting reaction mass was diluted with ethyl acetate (500 mL), washed with water (2×200 mL), dried over sodium sulfate and concentrated under reduced pressure. The crude product obtained upon evaporation of volatiles was purified through column chromatography to obtain product 46-7 as a colourless wax 4.1 g (61%).

Step 5: Preparation of (2S)-1-(2-{[(2S)-1-[2-(acetyloxy)-N-tert-butylacetamido]-3-{[4-(morpholin-4-yl)-1,2,5-thiadiazol-3-yl]oxy}propan-2-yl]oxy}-2-oxoethoxy)-1-oxopropan-2-yl (2S)-2-hydroxypropanoate (46-8): To a solution of (2S)-1-(2-{[(2S)-1-[2-(acetyloxy)-N-tert-butylacetamido]-3-{[4-(morpholin-4-yl)-1,2,5-thiadiazol-3-yl]oxy}propan-2-yl]oxy}-2-oxoethoxy)-1-oxopropan-2-yl (2S)-2-[(tert-butyldimethylsilyl)oxy]propanoate 46-7 (4.1 g, 5.60 mmol) in 1,4 dioxane (30 mL) was added 1N HCl (5.6 mL, 5.60 mmol) at 0° C. The reaction mixture was allowed to stir at 25-30° C. over a period of 2 h. The resulting reaction mass was diluted with ethyl acetate (500 mL), washed with water (2×200 mL), dried over sodium sulfate and concentrated under reduced pressure. The crude product obtained upon evaporation of volatiles was purified by silica gel column chromatography to obtain product 46-8 as a colourless wax 2.2 g (64%).

Step 6: Preparation (2S)-1-(2-{[(2S)-1-[2-(acetyloxy)-N-tert-butylacetamido]-3-{[4-(morpholin-4-yl)-1,2,5-thiadiazol-3-yl]oxy}propan-2-yl]oxy}-2-oxoethoxy)-1-oxopropan-2-yl (2S)-2-[(2-chloroacetyl)oxy]propanoate (46-10): To a solution of (2S)-1-(2-{[(2S)-1-[2-(acetyloxy)-N-tert-butylacetamido]-3-{[4-(morpholin-4-yl)-1,2,5-thiadiazol-3-yl]oxy}propan-2-yl]oxy}-2-oxoethoxy)-1-oxopropan-2-yl (2S)-2-hydroxypropanoate 46-8 (2.2 g, 3.23 mmol) in dichloromethane (40 mL) were added chloroacetyl chloride 46-9 (0.66 mL, 8.09 mmol) and 4-Dimethylaminopyridine (0.039 g, 0.323 mmol) at 0-5° C. The reaction mass was stirred at 25-30° C. for 4 h. The reaction mass was diluted with ethyl acetate (400 mL), washed with water (100 mL), dried over sodium sulfate and concentrated under reduced pressure. The crude product obtained upon evaporation of volatiles was purified through silica gel column chromatography to obtain product 46-10 as a colourless wax 2.0 g (91%).

Step 7: Preparation of 1-(2-{[(2S)-1-[2-(acetyloxy)-N-tert-butylacetamido]-3-{[4-(morpholin-4-yl)-1,2,5-thiadiazol-3-yl]oxy}propan-2-yl]oxy}-2-oxoethoxy)-1-oxopropan-2-yl 2-[(2-{4-chloro-2-[(furan-2-ylmethyl)amino]-5-sulfamoylbenzoyloxy}acetyl)oxy]propanoate (Compound 42): To a solution of 4-chloro-2-[(furan-2-ylmethyl)amino]-5-sulfamoylbenzoic acid 46-11 (1.6 g, 4.83 mmol) in N,N-Dimethylformamide (2.7 mL) were added potassium carbonate (0.79 g, 5.79 mmol), TBAI (0.17 g, 0.483 mmol) and (2S)-1-(2-{[(2S)-1-[2-(acetyloxy)-N-tert-butylacetamido]-3-{[4-(morpholin-4-yl)-1,2,5-thiadiazol-3-yl]oxy}propan-2-yl]oxy}-2-oxoethoxy)-1-oxopropan-2-yl (2S)-2-[(2-chloroacetyl)oxy]propanoate 46-10 (3.69 g, 5.32 mmol) at 0-5° C. The reaction mixture was allowed to stir at 25-30° C. over a period of 2 h. The resulting reaction mass was diluted with ethyl acetate (400 mL), washed with water (200 mL), dried over sodium sulfate and concentrated under reduced pressure. The crude product obtained upon evaporation of volatiles was purified by silica gel column chromatography to obtain product Compound 42 as a colourless wax 1.4 g (29%). ¹H-NMR (400 MHz, DMSO-d6) δ 8.47 (s, 1H), 8.35 (t, 1H), 7.62 (d, J=1 Hz, 1H), 7.40 (s, 2H), 7.14 (s, 1H), 6.44-6.34 (m, 2H), 5.52-5.42 (m, 1H), 5.30-5.16 (m, 2H), 5.03 (s, 2H), 4.96-4.77 (m, 3H), 4.67-4.54 (m, 4H), 4.49-4.41 (m, 1H), 3.71-3.52 (m, 6H), 3.42-3.3 (m, 4H), 2.08 (s, 3H), 1.50-1.41 (m, 6H), 1.33 (s, 9H). MS m/z [M+H]⁺ 991.3.

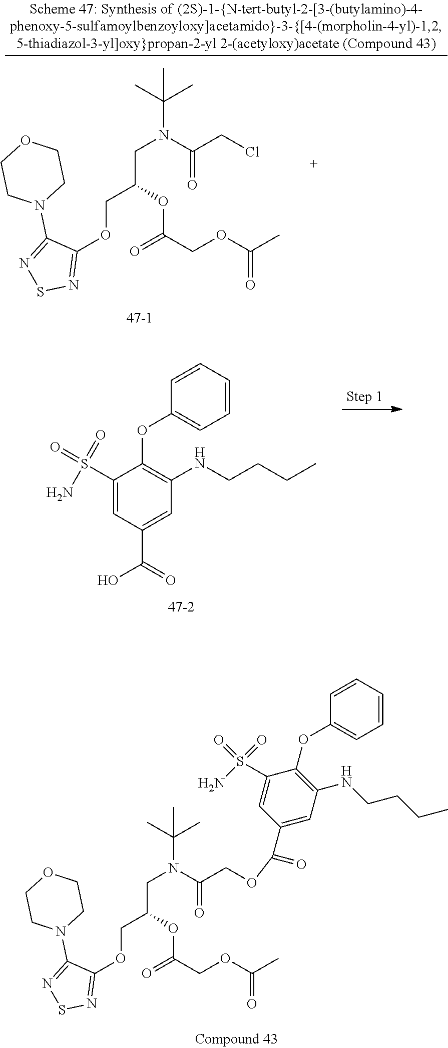 [SMILES: CC(=O)OCC(=O)O[C@H](COc1nsnc1N1CCOCC1)CN(C(=O)CCl)C(C)(C)C ; CCCCNc1cc(C(=O)O)cc(S(N)(=O)=O)c1Oc1ccccc1 ; CCCCNc1cc(C(=O)OCC(=O)N(C[C@@H](COc2nsnc2N2CCOCC2)OC(=O)COC(C)=O)C(C)(C)C)cc(S(N)(=O)=O)c1Oc1ccccc1]

Step 1: Preparation of (2S)-1-{N-tert-butyl-2-[3-(butylamino)-4-phenoxy-5-sulfamoylbenzoyloxy]acetamido}-3-{[4-(morpholin-4-yl)-1,2,5-thiadiazol-3-yl]oxy}propan-2-yl 2-(acetyloxy)acetate (Compound 43):

To a solution of 3-(butylamino)-4-phenoxy-5-sulfamoylbenzoic acid 47-2 (1.7 g, 4.67 mmol) in N,N-Dimethylformamide (5.1 mL) were added potassium carbonate (0.83 g, 6.071 mmol), TBAI (0.17 g, 0.467 mmol) and (2S)-1-(N-tert-butyl-2-chloroacetamido)-3-{[4-(morpholin-4-yl)-1,2,5-thiadiazol-3-yl]oxy}propan-2-yl 2-(acetyloxy)acetate 47-1 (2.9 g, 6.07 mmol) at 0-5° C. The reaction mixture was allowed to stir at 25-30° C. over a period of 3 h. The resulting reaction mass was diluted with ethyl acetate (400 mL), washed with water (200 mL), dried over sodium sulfate and concentrated under reduced pressure. The crude product obtained upon evaporation of volatiles was purified by silica gel column chromatography to obtain product Compound 43 as a colourless wax 1.5 g (39%). ¹H-NMR (400 MHz, DMSO-d6) δ 7.76 (d, J=2 Hz, 1H), 7.45 (d, J=2 Hz 1H), 7.39 (s, 2H), 7.27 (t, J=8 Hz, 2H), 7.02 (t, J=8 Hz, 1H), 6.86 (d, J=8 Hz, 2H), 5.54-5.44 (m, 1H), 5.23-5.12 (m, 2H), 5.04 (d, 1H), 4.84-4.74 (m, 2H), 4.66-4.59 (m, 1H), 4.54-4.46 (m, 1H), 3.75-3.62 (m, 6H), 3.48-3.32 (m, 4H), 3.07 (q, 2H), 2.11 (s, 3H), 1.42-1.25 (m, 11H), 1.14-1.03 (m, 2H), 0.77 (t, 3H). MS m/z [M+H]⁺ 822.2.

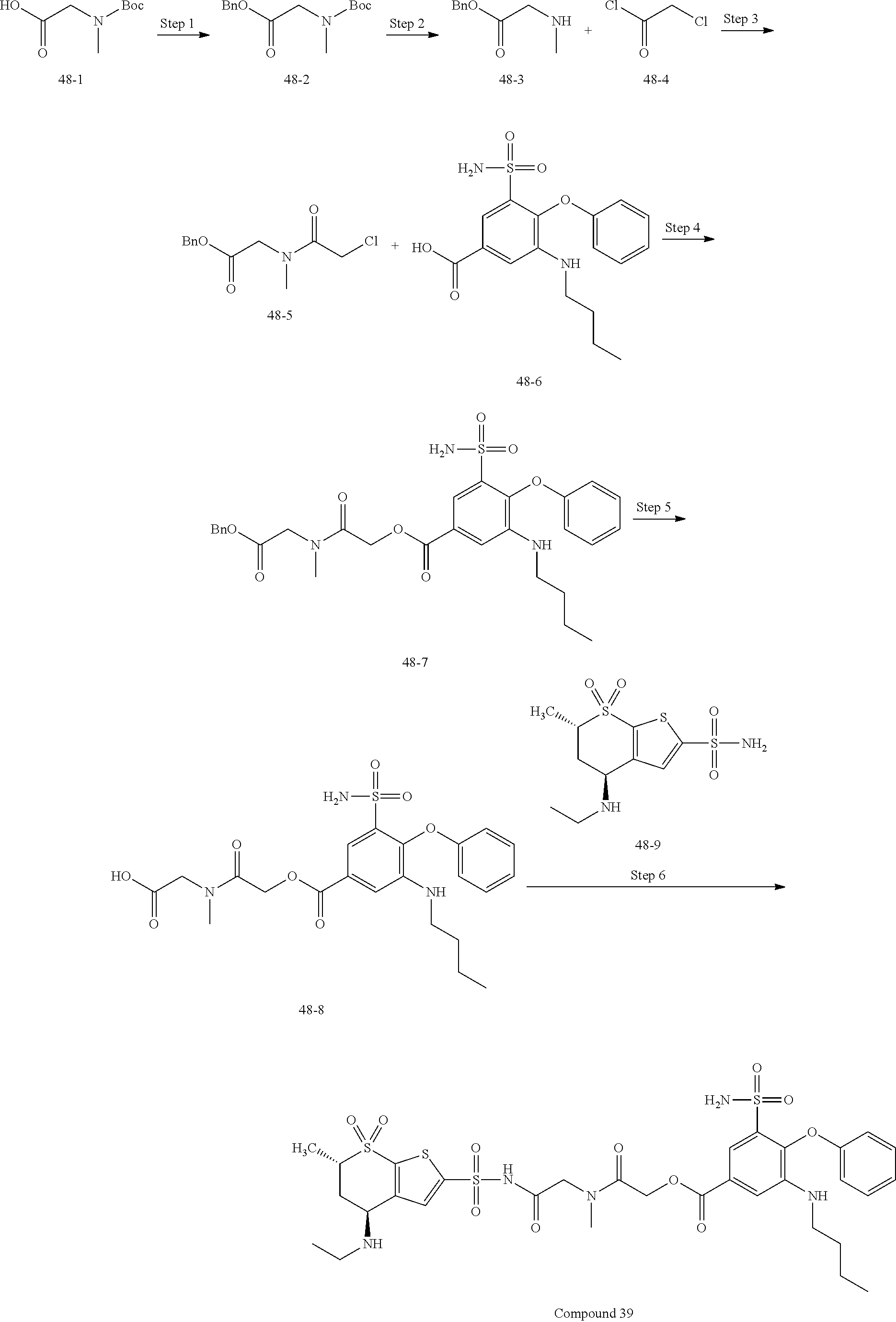

Step 1: Preparation of benzyl [(tert-butoxycarbonyl)(methyl)amino]acetate (48-2): To a solution of [(tert-butoxycarbonyl)(methyl)amino]acetic acid 48-1 (50 g, 0.264 mol) in dichloromethane (500 mL) were added EDC.HCl (75.71 g, 0.396 mol), benzyl alcohol (22.86 g, 0.211 mol) and 4-Dimethylaminopyridine (3.22 g, 0.026 mol) at 0-5° C. The reaction mass was allowed to stir at 25-30° C. for 2 h. The reaction mass was diluted with ethyl acetate (1.5 L) and washed with water (750 mL). The organic layer was dried over sodium sulfate and concentrated under reduced pressure. The residue obtained was purified by silica (60-120 mesh) column chromatography to give product 48-2 as a waxy compound 54g (73%).

Step 2: Preparation of benzyl (methylamino)acetate (48-3): To a solution of benzyl [(tert-butoxycarbonyl)(methyl)amino]acetate 48-2 (54 g, 0.193 mol) in dichloromethane (540 mL) was added TFA (216 mL, 4V) at 0-5° C. The reaction mass was allowed to stir at 25-30° C. for 1 h. The reaction mass was concentrated under reduced pressure at 45° C. to give product 48-3 as a brown colour wax 85.0 g (crude compound 48-3 was carried as such into next step without any purification).

Step 3: Preparation of benzyl [(chloroacetyl)(methyl)amino]acetate (48-5): To a solution of benzyl (methylamino)acetate 4-83 (85.0 g, 0.474 mol) in dichloromethane (850 mL) were added triethylamine (194.57 mL, 1.422 mol) 4-dimethyl amino pyridine (5.78 g, 0.047 mol) and chloroacetyl chloride 48-4 (56.56 mL, 0.711 mol) drop-wise at 0-5° C. The resulting reaction mixture was allowed to stir at 25-30° C. for 1 h. The reaction mass was diluted with ethyl acetate (1.5 L) and washed with water (2×750 mL). The organic layer was dried over sodium sulfate and concentrated under reduced pressure. The residue obtained was purified by silica gel (230-400 mesh) column chromatography to give product 48-5 as a colourless wax 23.0 g (18.9%).

Step 4: Preparation of benzyl 2-{2-[3-(butylamino)-4-phenoxy-5-sulfamoylbenzoyloxy]-N-methylacetamido}acetate (48-7): To a solution of bumetanide 48-6 (19 g, 52.13 mmol) in N,N-Dimethylformamide (100 mL) were added K₂CO₃ (8.64 g, 62.55 mmol), TBAI (1.92 g, 5.21 mmol) and benzyl [(chloroacetyl)(methyl)amino]acetate 48-5 (17.33 g, 67.78 mmol) at 0-5° C. The resulting reaction mixture was allowed to stir at 25-30° C. for 4 h. The reaction mass was diluted with ethyl acetate (1.0 L) and washed with water (2×500 mL). The organic layer was dried over sodium sulfate and concentrated under reduced pressure. The residue obtained was purified by silica gel (230-400 mesh) column chromatography to give product 48-7 as an off white solid 21.5 g (69%).

Step 5: Preparation of 2-{2-[3-(butylamino)-4-phenoxy-5-sulfamoylbenzoyloxy]-N-methylacetamido}acetic acid (48-8): Pd/C (2.2 g, 20% w/w) was charged to a solution of benzyl 2-{2-[3-(butylamino)-4-phenoxy-5-sulfamoylbenzoyloxy]-N-methylacetamido} acetate 48-7 (21.5 g, 36.83 mmol) in methanol/dichloromethane (3:7, 215 mL) taken in a Parr-shaker vessel. The reaction mixture was hydrogenated with 5 kg/cm² H₂ pressure at 25-30° C. for 1 h. The reaction progress was monitored by TLC. After completion of the reaction, reaction mass was filtered through celite bed and filtrate was concentrated under reduced pressure to give product 48-8 as an off white solid 15.5 g (85.3%).

Step 6: Preparation of {[({[(2S,4S)-4-(ethylamino)-2-methyl-1,1-dioxo-2H,3H,4H-1λ⁶-thieno[2,3-b]thiopyran-6-yl]sulfonyl}carbamoyl)methyl](methyl)carbamoyl}methyl 3-(butylamino)-4-phenoxy-5-sulfamoylbenzoate (Compound 39): To a solution of (2S,4S)-4-(ethylamino)-2-methyl-1,1-dioxo-2H,3H,4H-1λ⁶-thieno[2,3-b]thiopyran-6-sulfonamide 48-9 (1.2 g, 3.34 mmol) in dichloromethane (24 mL) were added DIPEA (0.59 mL, 3.34 mmol), 2-{2-[3-(butylamino)-4-phenoxy-5-sulfamoylbenzoyloxy]-N-methylacetamido}acetic acid 48-8 (2.1 g, 4.34 mmol), EDCI.HCl (0.95 g, 5.01 mmol) and 4-Dimethylaminopyridine (0.04 g, 0.33 mmol) at 0-5° C. The reaction mass was allowed to stir at 25-30° C. for 16 h. The reaction mass was diluted with ethyl acetate (300 mL), washed with water (200 mL), dried over sodium sulfate and concentrated under reduced pressure. The crude product obtained upon evaporation of volatiles was purified by reverse phase column chromatography to obtain product Compound 39 as a white solid 1.0 g (38%). ¹H NMR (400 MHz, DMSO-d₆) δ 8.8 (bs, 2H), 7.80-7.64 (m, 2H), 7.46-7.35 (m, 3H), 7.26 (t, J=8 Hz, 2H), 7.01 (t, J=8 Hz, 1H), 6.87-6.81 (m, 2H), 5.20-5.13 (m, 1H), 5.10 & 4.92 (2s, 2H), 4.7-4.5 (m, 1H), 4.02-3.73 (m, 3H), 3.3-3.0 (m, 4H), 2.96 & 2.78 (2s, 3H), 2.6-2.5 (m, 2H), 1.42-1.30 (m, 5H), 1.27-1.01 (m, 5H), 0.77 (t, 3H); MS m/z [M+H]⁺ 800.5.

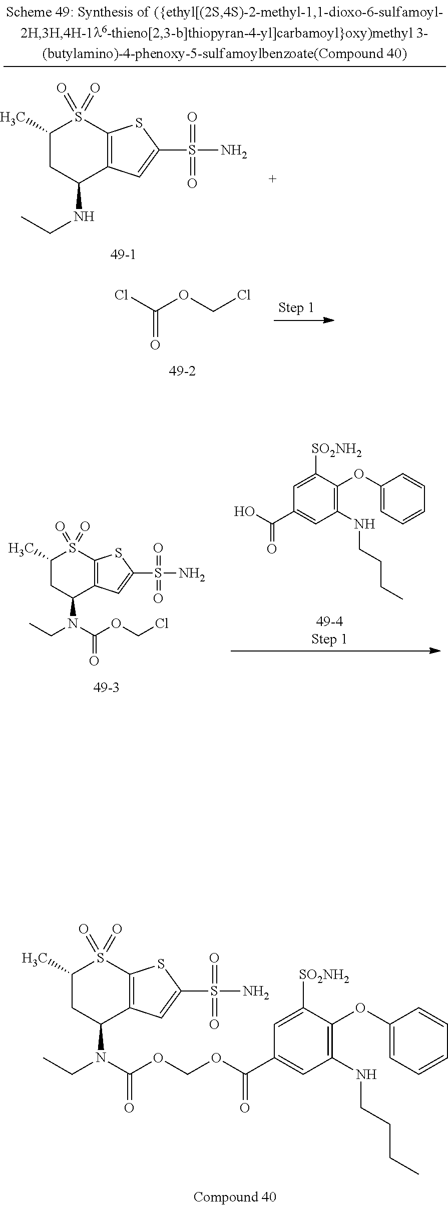 [SMILES: CCN[C@H]1C[C@H](C)S(=O)(=O)c2sc(S(N)(=O)=O)cc21 ; O=C(Cl)OCCl ; CCN(C(=O)OCCl)[C@H]1C[C@H](C)S(=O)(=O)c2sc(S(N)(=O)=O)cc21 ; CCCCNc1cc(C(=O)O)cc(S(N)(=O)=O)c1Oc1ccccc1 ; CCCCNc1cc(C(=O)OCOC(=O)N(CC)[C@H]2C[C@H](C)S(=O)(=O)c3sc(S(N)(=O)=O)cc32)cc(S(N)(=O)=O)c1Oc1ccccc1]

Step 1: Preparation of chloromethyl ethyl[(4S,6S)-6-methyl-7,7-dioxo-2-sulfamoyl-4,5,6,7-tetrahydro-716-thieno[2,3-b]thiopyran-4-yl]carbamate (49-3): To a solution of dorzolamide 49-1 (1.4 g, 3.88 mmol) in dichloromethane (28 mL) was added N,N-Diisopropylethylamine (1.41 mL, 7.7 mmol) at 25-30° C. After 30 min, chloromethyl carbonochloridate 49-2 (0.38 g, 4.2 mmol) was added at 0-5° C. and the reaction mixture was allowed to stir for 1 h. The resulting reaction mass was diluted with ethyl acetate (300 mL) and washed with water (100 mL×2), organic layer was dried over sodium sulfate and concentrated under reduced pressure to obtain compound 49-3 as an off white solid 0.75 g (46%). The crude compound was taken forward to next step without any purification.

Step 2: Preparation of ({ethyl[(2S,4S)-2-methyl-1,1-dioxo-6-sulfamoyl-2H,3H,4H-1λ⁶-thieno[2,3-b]thiopyran-4-yl]carbamoyl}oxy)methyl 3-(butylamino)-4-phenoxy-5-sulfamoylbenzoate (Compound 40): To a solution of chloromethyl ethyl[(4S,6S)-6-methyl-7,7-dioxo-2-sulfamoyl-4,5,6,7-tetrahydro-716-thieno[2,3-b]thiopyran-4-yl]carbamate 49-3 (0.3 g, 0.71 mmol) in N,N-Dimethylformamide (3 mL) were added sodium iodide (0.162 g, 1.07 mmol), 3-(butylamino)-4-phenoxy-5-sulfamoylbenzoic acid 49-4 (0.393 g, 1.07 mmol) and triethylamine (0.20 mL, 1.43 mmol) at 0-5° C. The reaction mixture was allowed to stir at 55° C. over a period of 3 h. The resulting reaction mass was diluted with ethyl acetate (100 mL) and washed with water (50 mL×2), organic layer was dried over sodium sulfate and concentrated under reduced pressure. The crude compound was purified by reverse phase column chromatography to obtain product Compound 40 as a white solid 0.29 g (28%). ¹H NMR (400 MHz, DMSO-d₆) δ 8.1-7.3 (m, 7H), 7.27 (t, J=8 Hz, 2H), 7.02 (t, J=8 Hz, 1H), 6.85 (d, J=8 Hz, 2H), 6.0-5.7 (m, 2H), 5.26-5.10 (m, 2H), 3.96-3.81 (m, 1H), 3.4-3.0 (m, 4H), 2.9-2.7 (m, 1H), 2.5-2.4 (m, 1H), 1.43-1.30 (m, 5H), 1.18-1.04 (m, 5H), 0.77 (t, 3H); MS m/z [M+H]⁺ 745.6.

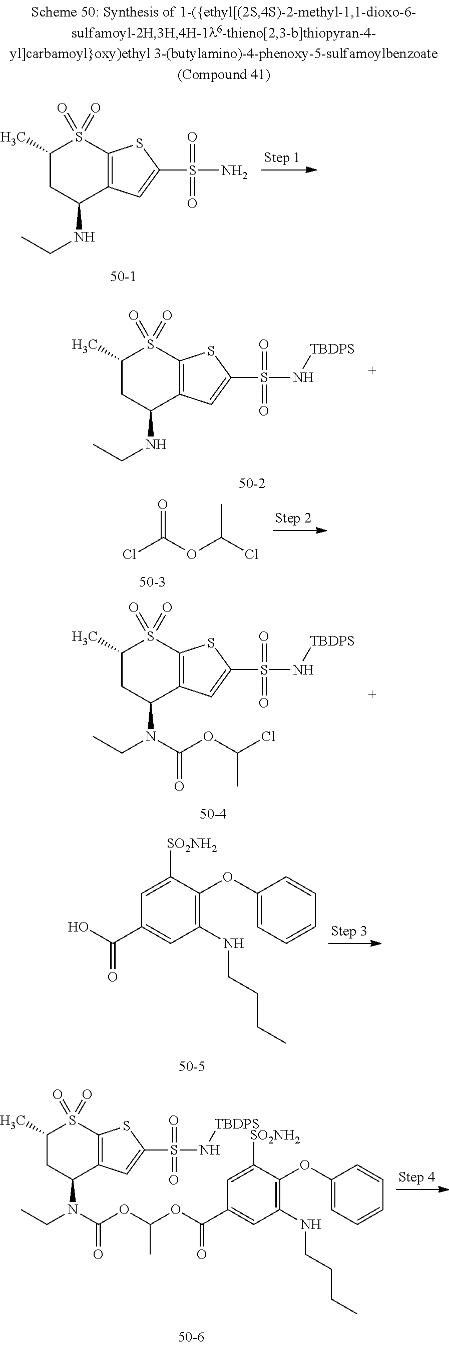

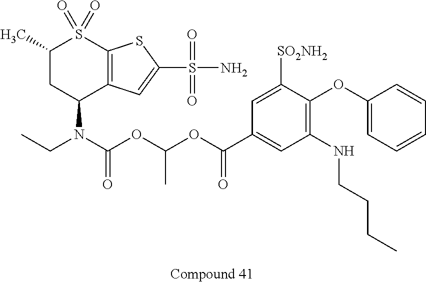 [SMILES: CCCCNc1cc(C(=O)OC(C)OC(=O)N(CC)[C@H]2C[C@H](C)S(=O)(=O)c3sc(S(N)(=O)=O)cc32)cc(S(N)(=O)=O)c1Oc1ccccc1]

Step 1: Preparation of (14-1): (2S,4S)—N-(tert-butyldiphenylsilyl)-4-(ethylamino)-2-methyl-1,1-dioxo-2H,3H,4H-1λ⁶-thieno[2,3-b]thiopyran-6-sulfonamide (50-2): To a solution of dorzolamide 50-1 (2.0 g, 5.55 mmol) in dichloromethane (20 mL) were added N,N-Diisopropylethylamine (1.99 mL, 11.11 mmol), tertiary-butyl diphenylsilyl chloride (1.58 mL, 6.11 mmol), and 4-Dimethylaminopyridine (67 mg, 0.55 mmol) at 0-5° C. The reaction mixture was allowed to stir at 25-30° C. over a period of 2 h. The resulting reaction mass was diluted with ethyl acetate (200 mL), washed with water (2×100 mL), dried over sodium sulfate and concentrated under reduced pressure. The crude product obtained upon evaporation of volatiles was purified through silica gel (230-400 mesh) column chromatography to obtain product 50-2 as a white solid 2.4 g (76%).

Step 2: Preparation of (14-3): 1-chloroethyl N-[(2S,4S)-6-[(tert-butyldiphenylsilyl)sulfamoyl]-2-methyl-1,1-dioxo-2H,3H,4H-1λ⁶-thieno[2,3-b]thiopyran-4-yl]-N-ethylcarbamate (50-4): To a solution of (2S,4S)—N-(tert-butyldiphenylsilyl)-4-(ethylamino)-2-methyl-1,1-dioxo-2H,3H,4H-1λ⁶-thieno[2,3-b]thiopyran-6-sulfonamide 50-2 (1.0 g, 1.77 mmol) in dichloromethane (10 mL) were added N,N-Diisopropylethylamine (0.636 mL, 3.55 mmol) and 1-chloroethyl carbonochloridate 50-3 (0.191 mL, 1.77 mmol) at 0° C. The reaction mixture was allowed to stir at 25-30° C. over a period of 1 h. The resulting reaction mass was diluted with ethyl acetate (100 mL), washed with water (2×80 mL), dried over sodium sulfate and concentrated under reduced pressure to afford 50-4 as a colourless wax 1.0 g. The crude product obtained was taken as such into next step without any further purification.

Step 3: Preparation of 1 1-({[(2S,4S)-6-[(tert-butyldiphenylsilyl)sulfamoyl]-2-methyl-1,1-dioxo-2H,3H,4H-1λ⁶-thieno[2,3-b]thiopyran-4-yl](ethyl)carbamoyl}oxy)ethyl 3-(butylamino)-4-phenoxy-5-sulfamoylbenzoate (50-6): To a solution of bumetanide 50-5 (0.408 g, 1.12 mmol) in THF (10 mL) were added triethylamine (0.20 mL, 1.49 mmol), NaI (0.167 g, 1.12 mmol) and 1-chloroethyl N-[(2S,4S)-6-[(tert-butyldiphenylsilyl)sulfamoyl]-2-methyl-1,1-dioxo-2H,3H,4H-1λ⁶-thieno[2,3-b]thiopyran-4-yl]-N-ethylcarbamate 50-4 (0.5 g, 0.74 mmol) at 0-5° C. The reaction mixture was allowed to stir at 55° C. for 2 h. The resulting reaction mass was diluted with ethyl acetate (100 mL), washed with water (2×50 mL), dried over sodium sulfate and concentrated under reduced pressure to afford 50-6 as an off white wax 0.4 g. The crude product obtained was taken as such into next step without any further purification.

Step 4: Preparation of 1-({ethyl[(2S,4S)-2-methyl-1,1-dioxo-6-sulfamoyl-2H,3H,4H-1)⁶-thieno[2,3-b]thiopyran-4-yl]carbamoyl}oxy)ethyl 3-(butylamino)-4-phenoxy-5-sulfamoylbenzoate (Compound 41): To a solution of 1 1-({[(2S,4S)-6-[(tert-butyldiphenylsilyl)sulfamoyl]-2-methyl-1,1-dioxo-2H,3H,4H-1λ⁶-thieno[2,3-b]thiopyran-4-yl](ethyl)carbamoyl}oxy)ethyl 3-(butylamino)-4-phenoxy-5-sulfamoylbenzoate 50-6 (0.4 g, 0.401 mmol) in THF (10 mL) was added TBAF (1M in THF, 0.401 mL, 0.401 mmol) at 0-5° C. The reaction mixture was allowed to stir at 25-30° C. over a period of 3 h. The resulting reaction mass was diluted with ethyl acetate (80 mL), washed with water (2×40 mL), dried over sodium sulfate and concentrated under reduced pressure. The crude product obtained upon evaporation of volatiles was purified by preparative HPLC to give Compound 41 as an off white solid 140 mg (46%). ¹H NMR (400 MHz, DMSO-d₆) δ 8.3-7.5 (m, 4H), 7.47-7.20 (m, 5H), 7.05-6.68 (m, 4H), 5.32-4.75 (m, 2H), 3.95-3.76 (m, 1H), 3.6-2.9 (m, 4H), 2.9-2.7 (m, 1H), 2.5-2.4 (m, 1H), 1.7-1.0 (m, 13H), 0.80-0.72 (m, 3H); MS m/z [M+H]⁺ 759.4.

### Example 13: Synthesis of Bumetanide Glycolates

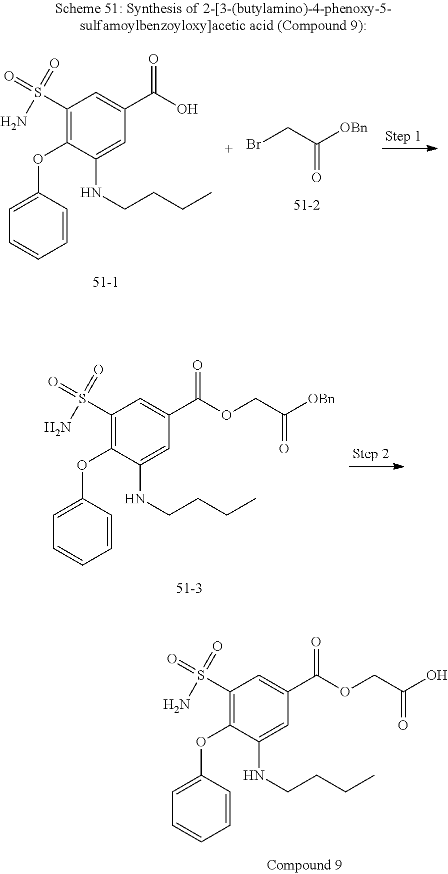 [SMILES: CCCCNc1cc(C(=O)O)cc(S(N)(=O)=O)c1Oc1ccccc1 ; O=C(CBr)OCc1ccccc1 ; CCCCNc1cc(C(=O)OCC(=O)OCc2ccccc2)cc(S(N)(=O)=O)c1Oc1ccccc1 ; CCCCNc1cc(C(=O)OCC(=O)O)cc(S(N)(=O)=O)c1Oc1ccccc1]

Step 1: Preparation of benzyl 2-[3-(butylamino)-4-phenoxy-5-sulfamoylbenzoyloxy]acetate (51-3): To a solution of bumetanide 51-1 (2.0 g, 5.48 mmol) in N,N-Dimethylformamide (20 mL) were added potassium carbonate (1.136 g, 8.23 mmol) and benzyl 2-bromoacetate 51-2 (0.698 mL, 4.39 mmol) at 0° C. The reaction mixture was allowed to stir at 25-30° C. over a period of 2 h. The resulting reaction mass was diluted with ethyl acetate (250 mL), washed with water (2×150 mL), organic layer was dried over sodium sulfate and concentrated under reduced pressure. The crude product obtained upon evaporation of volatiles was purified through silica gel (230-400 mesh) column chromatography to obtain product 51-3 as an off white solid 2.0 g (71%).

Step 2: Preparation of 2-[3-(butylamino)-4-phenoxy-5-sulfamoylbenzoyloxy]acetic acid (Compound 9): To a 100 mL autoclave vessel were added a solution of benzyl 2-[3-(butylamino)-4-phenoxy-5-sulfamoylbenzoyloxy]acetate 51-3 (2.0 g, 3.90 mmol) in methanol (20 mL) and 10% Pd/C (400 mg, 50% wet) at 25-30° C. The reaction mixture was stirred at 25-30° C. under hydrogen pressure (5 kg/cm²) over a period of 1 h. After completion of the reaction, the reaction mixture was filtered through celite bed. Then volatiles were evaporated under reduced pressure to obtain crude compound. The crude compound was stirred with diethyl ether (20 mL) at 0-5° C. The solid precipitate obtained was filtered and dried under high vacuum to afford Compound 9 as an off white solid 1.4 g (85%). ¹H NMR (400 MHz, DMSO-d₆) δ 13.3 (bs, 1H), 7.74 (d, J=2 Hz 1H), 7.45-7.36 (m, 3H), 7.27 (t, 2H), 7.01 (t, 1H), 6.85 (dd, 2H), 5.20 (t, 1H), 4.84 (s, 2H), 3.07 (q, 2H), 1.37 (quintet, 2H), 1.16-1.03 (m, 2H), 0.77 (t, 3H); MS m/z [M+H]⁺ 423.7

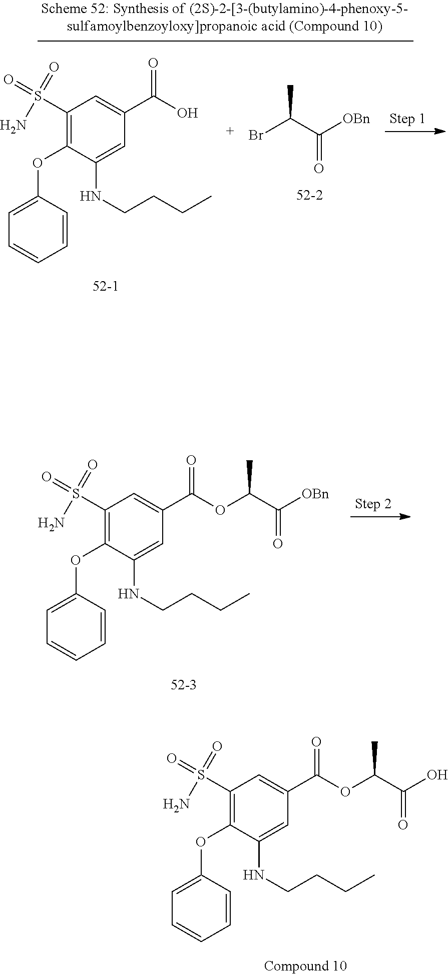 [SMILES: CCCCNc1cc(C(=O)O)cc(S(N)(=O)=O)c1Oc1ccccc1 ; C[C@H](Br)C(=O)OCc1ccccc1 ; CCCCNc1cc(C(=O)O[C@@H](C)C(=O)OCc2ccccc2)cc(S(N)(=O)=O)c1Oc1ccccc1 ; CCCCNc1cc(C(=O)O[C@@H](C)C(=O)O)cc(S(N)(=O)=O)c1Oc1ccccc1]

Step 1: Preparation of benzyl (2S)-2-[3-(butylamino)-4-phenoxy-5-sulfamoylbenzoyloxy]propanoate (52-3): To a solution of bumetanide 52-1 (2.0 g, 5.48 mmol) in N,N-Dimethylformamide (15 mL) were added EDC.HCl (1.57 g, 8.23 mmol), hydroxybenzotriazole (0.741 g, 5.48 mmol), 4-Dimethylaminopyridine (0.134 g, 1.09 mmol) and benzyl (2S)-2-hydroxypropanoate 52-2 (1.48 g, 8.23 mmol) at 0-5° C. The reaction mixture was allowed to stir at 80° C. over a period of 16 h. The resulting reaction mass was diluted with ethyl acetate (200 mL), washed with water (2×150 mL), dried over sodium sulfate and concentrated under reduced pressure. The crude product obtained upon evaporation of volatiles was stirred with methanol (10 mL), the solid precipitated out was filtered and dried to obtain product 52-3 as a white solid 1.3 g (45%).

Step 2: Preparation of (2S)-2-[3-(butylamino)-4-phenoxy-5-sulfamoylbenzoyloxy]propanoic acid (Compound 10): To a 100 mL autoclave vessel were added a solution of benzyl (2S)-2-[3-(butylamino)-4-phenoxy-5-sulfamoylbenzoyloxy]propanoate 52-3 (1.3 g, 2.46 mmol) in methanol (13 mL) and 10% Pd/C (260 mg, 50% wet) at 25-30° C. The reaction mixture was stirred at 25-30° C. under hydrogen pressure (5 kg/cm²) over a period of 1 h. After completion of the reaction, the reaction mixture was filtered through celite bed. The volatiles were evaporated under reduced pressure to obtain crude. The crude compound was stirred with diethyl ether (13 mL) at 0-5° C. The solid precipitate obtained was filtered and dried to obtain product Compound 10 as a grey solid 750 mg (70%). ¹H NMR (400 MHz, DMSO-d₆) δ 13.3 (bs, 1H), 7.72 (d, J=1 Hz 1H), 7.46-7.37 (m, 3H), 7.27 (t, 2H), 7.01 (t, 1H), 6.85 (dd, 2H), 5.24-5.12 (m, 2H), 3.12-3.01 (m, 2H), 1.54 (d, 3H), 1.41-1.32 (m, 2H), 1.17-1.02 (m, 2H), 0.78 (t, 3H); MS m/z [M+H]⁺ 437.8.

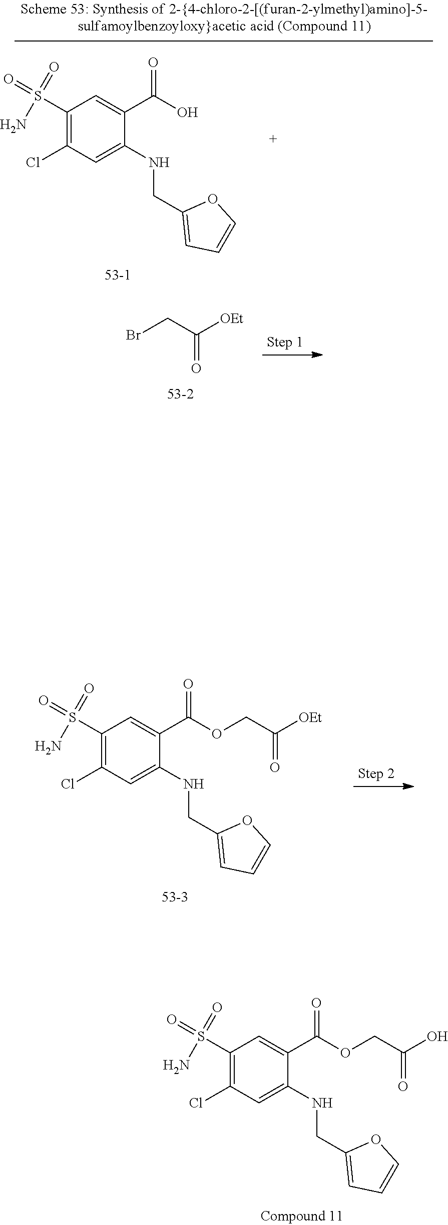 [SMILES: NS(=O)(=O)c1cc(C(=O)O)c(NCc2ccco2)cc1Cl ; CCOC(=O)CBr ; CCOC(=O)COC(=O)c1cc(S(N)(=O)=O)c(Cl)cc1NCc1ccco1 ; NS(=O)(=O)c1cc(C(=O)OCC(=O)O)c(NCc2ccco2)cc1Cl]

Step 1: Preparation of ethyl 2-{4-chloro-2-[(furan-2-ylmethyl)amino]-5-sulfamoylbenzoyloxy}acetate (53-3): To a solution of furosemide 53-1 (5 g, 15 mmol) in N,N-dimethylformamide (50 mL) were added potassium carbonate (3.13 g, 22 mmol) and ethyl 2-bromoacetate 53-2 (1.5 mL, 13 mmol) at 0° C. The reaction mixture was allowed to stir at 25-30° C. over a period of 1 h. The resulting reaction mass was diluted with ethyl acetate (300 mL), washed with water (2×150 mL), dried over sodium sulfate and concentrated under reduced pressure. The crude product obtained upon evaporation of volatiles was stirred with ethanol (20 mL) at 0-5° C. The solid precipitate was collected by filtration and dried under high vacuum to obtain product 53-3 as a pale yellow solid 4.6 g (73%).

Step 2: Preparation of 2-{4-chloro-2-[(furan-2-ylmethyl)amino]-5-sulfamoylbenzoyloxy}acetic acid (Compound 11): To a solution of ethyl 2-{4-chloro-2-[(furan-2-ylmethyl)amino]-5-sulfamoylbenzoyloxy}acetate 53-3 (2.8 g, 6.71 mmol) in ethanol (28 mL) was added 1.0 N aqueous sodium hydroxide solution (6.7 mL, 6.71 mmol) drop-wise at 0° C. The reaction mixture was allowed to stir at 25-30° C. over a period of 16 h. The resulting reaction mass was concentrated under reduced pressure. The residue obtained was diluted with water (28 mL) and washed with diethyl ether (2×50 mL) to remove the impurities. The aqueous phase was acidified (pH=2) with 1N hydrochloric acid (8 mL). The solid precipitate obtained was collected by filtration, washed with diethyl ether and dried under high vacuum to afford product Compound 11 as a white solid 600 mg (23%). ¹H NMR (400 MHz, DMSO-d₆) δ 13.24 (bs, 1H), 8.47 (s, 1H), 8.40 (t, 1H), 7.63-7.61 (m, 1H), 7.39 (s, 2H), 7.13 (s, 1H), 6.43-6.34 (m, 2H), 4.83 (s, 2H), 4.61 (d, 2H); MS m/z [M−H]⁻ 387.1.

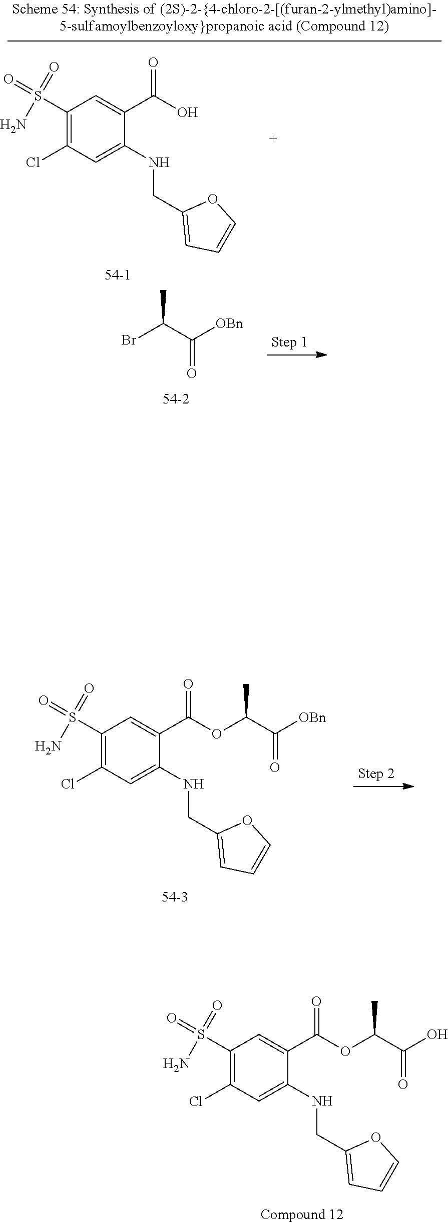 [SMILES: NS(=O)(=O)c1cc(C(=O)O)c(NCc2ccco2)cc1Cl ; C[C@H](Br)C(=O)OCc1ccccc1 ; C[C@H](OC(=O)c1cc(S(N)(=O)=O)c(Cl)cc1NCc1ccco1)C(=O)OCc1ccccc1 ; C[C@H](OC(=O)c1cc(S(N)(=O)=O)c(Cl)cc1NCc1ccco1)C(=O)O]

Step 1: Preparation of benzyl (2S)-2-{4-chloro-2-[(furan-2-ylmethyl)amino]-5-sulfamoylbenzoyloxy}propanoate (54-3): To a solution of furosemide 54-1 (2.5 g, 7.55 mmol) in tetrahydrofuran (12.5 mL) was added CDI (1.22 g, 7.55 mmol) and stirred at 25-30° C. over a period of 1 h. To the resulting solution, was added benzyl (2S)-2-hydroxypropanoate 54-2 (1.76 g, 9.81 mmol) followed by potassium tert-butoxide (1.01 g, 9.06 mmol) at 0° C. and the reaction mixture was allowed to stir at 0° C. for 1 h. The reaction mixture was diluted with water (150 mL), extracted with ethyl acetate (2×200 mL), combined organic layer was dried over sodium sulfate and concentrated under reduced pressure. The crude product obtained upon evaporation of volatiles was purified through silica gel (60-120 mesh) column chromatography (25-30% ethyl acetate in hexane) to obtain product 54-3 as an off white solid 1.87 g (50%).

Step 2: Preparation of (2S)-2-{4-chloro-2-[(furan-2-ylmethyl)amino]-5-sulfamoylbenzoyloxy}propanoic acid (Compound 12): To a solution of benzyl (2S)-2-{4-chloro-2-[(furan-2-ylmethyl)amino]-5-sulfamoylbenzoyloxy}propanoate 54-3 (1.5 g, 3.60 mmol) in THF (18 mL) was added 1.0 N aqueous sodium hydroxide solution (3.60 mL, 3.60 mmol) drop-wise at 0° C. The reaction mixture was allowed to stir at 25-30° C. over a period of 16 h. The resulting reaction mass concentrated under reduced pressure. The residue obtained was diluted with water (20 mL) and washed with diethyl ether (2×50 mL) to remove the impurities. The aqueous phase was acidified (p^{H}=2) with 1N hydrochloric acid (5 mL). The solid precipitate was collected by filtration, washed with diethyl ether and dried under high vacuum to obtain product Compound 12 as a white puffy solid 520 mg (37%). ¹H NMR (400 MHz, DMSO-d₆) δ 13.23 (bs, 1H), 8.45 (s, 1H), 8.41 (t, 1H), 7.63-7.61 (m, 1H), 7.39 (s, 2H), 7.12 (s, 1H), 6.42-6.34 (m, 2H), 5.14 (q, 1H), 4.60 (d, 2H), 1.51 (d, 3H); MS m/z [M−H]⁻ 401.1.

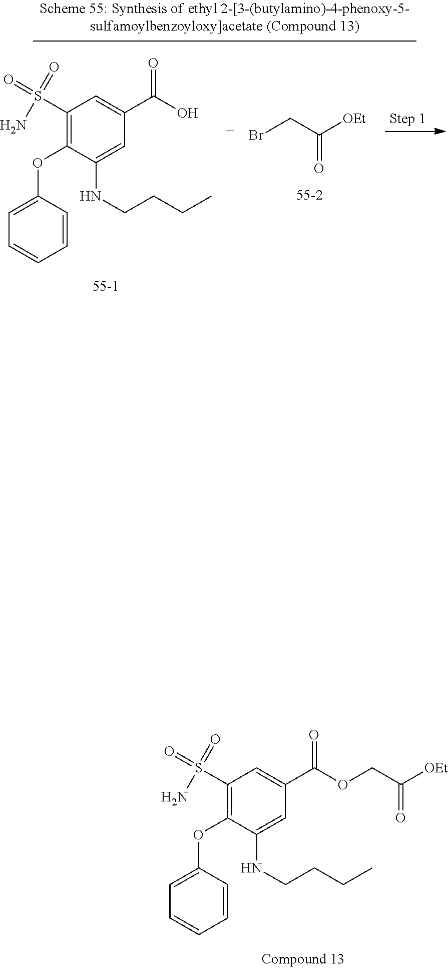 [SMILES: CCCCNc1cc(C(=O)O)cc(S(N)(=O)=O)c1Oc1ccccc1 ; CCOC(=O)CBr ; CCCCNc1cc(C(=O)OCC(=O)OCC)cc(S(N)(=O)=O)c1Oc1ccccc1]

Step 1: Preparation of ethyl 2-[3-(butylamino)-4-phenoxy-5-sulfamoylbenzoyloxy]acetate (Compound 13): To a solution of bumetanide 55-1 (1.2 g, 3.29 mmol) in N,N-dimethylformamide (12 mL) was added dry potassium carbonate (0.908 g, 6.58 mmol) followed by ethyl 2-bromoacetate 55-2 (0.364 mL, 3.29 mmol) at 0-5° C. The reaction mixture was allowed to stir at 25-30° C. for 2 h. The resulting reaction mixture was diluted with water (150 mL) and extracted with ethyl acetate (200 mL). The organic layer was further washed with water (2×150 mL), dried over sodium sulfate and concentrated under reduced pressure to give Compound 13 as a white puffy solid 1.2 g (80%). ¹H NMR (400 MHz, DMSO-d₆) δ 7.74 (d, J=2 Hz 1H), 7.46-7.39 (m, 3H), 7.27 (t, 2H), 7.02 (t, 1H), 6.85 (d, 2H), 5.21 (t, 1H), 4.95 (s, 2H), 4.18 (q, 2H), 3.11-3.01 (m, 2H), 1.37 (quintet, 2H), 1.23 (t, 3H), 1.01 (sextet, 2H), 0.77 (t, 3H); MS m/z [M+H]⁺ 452.2.

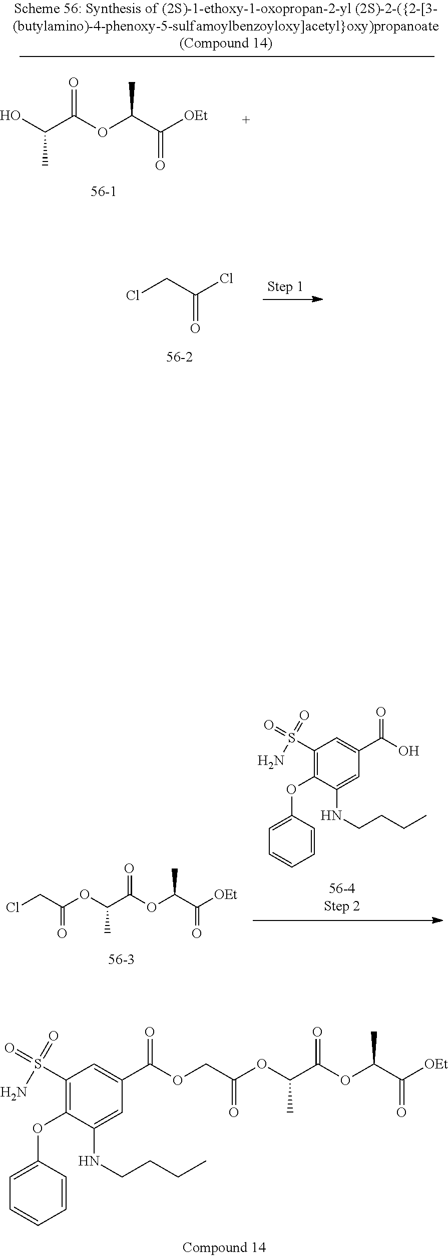 [SMILES: CCOC(=O)[C@H](C)OC(=O)[C@H](C)O ; O=C(Cl)CCl ; CCCCNc1cc(C(=O)O)cc(S(N)(=O)=O)c1Oc1ccccc1 ; CCOC(=O)[C@H](C)OC(=O)[C@H](C)OC(=O)CCl ; CCCCNc1cc(C(=O)OCC(=O)O[C@@H](C)C(=O)O[C@@H](C)C(=O)OCC)cc(S(N)(=O)=O)c1Oc1ccccc1]

Step 1: Preparation of (2S)-1-ethoxy-1-oxopropan-2-yl (2S)-2-[(2-chloroacetyl)oxy]propanoate (56-3): To a solution of (2S)-1-ethoxy-1-oxopropan-2-yl (2S)-2-hydroxypropanoate 56-1 (4.0 g, 21.03 mmol) in DCM (40 mL) was added triethylamine (9.10 mL, 63.09 mmol), followed by chloroacetyl chloride 56-2 (2.509 mL, 31.54 mmol) drop-wise at 0° C. The reaction mixture was allowed to stir at 0° C. to 25-30° C. over a period of 16 h. The resulting reaction mass was quenched with water (200 mL), extracted with ethyl acetate (2×250 mL), dried over sodium sulfate and concentrated under reduced pressure. The crude product obtained upon evaporation of volatiles was purified through silica gel (230-400 mesh) column (8-10% ethyl acetate in hexane) to give product 56-3 as pale yellow oil 4.0 g (71%).

Step 2: Preparation of (2S)-1-ethoxy-1-oxopropan-2-yl (2S)-2-({2-[3-(butylamino)-4-phenoxy-5-sulfamoylbenzoyloxy]acetyl}oxy)propanoate (Compound 14): To a solution of bumetanide 56-4 (10.0 g, 27.4 mmol) in N,N-Dimethylformamide (50 mL) were added K₂CO₃ (4.92 g, 35.67 mmol), TBAI (1.01 g, 2.74 mmol) and (2S)-1-ethoxy-1-oxopropan-2-yl (2S)-2-[(2-chloroacetyl)oxy]propanoate 56-3 (10.95 g, 41.16 mmol) at 0° C. The reaction mixture was allowed to stir at 25-30° C. for 2 h. The resulting reaction mass diluted with ethyl acetate (500 mL), washed with water (2×250 mL), dried over sodium sulfate and concentrated under reduced pressure. The crude product obtained upon evaporation of volatiles was purified through normal phase grace column chromatography (32% ethyl acetate in hexane) to give product Compound 14 as a pale yellow wax 9.5 g (58%). ¹H NMR (400 MHz, DMSO-d₆) δ 7.73 (d, J=2 Hz 1H), 7.46-7.39 (m, 3H), 7.27 (t, 2H), 7.02 (t, 1H), 6.84 (dd, 2H), 5.27-5.19 (m, 2H), 5.14-5.00 (m, 3H), 4.18-4.09 (m, 2H), 3.10-3.01 (m, 2H), 1.50 (t, 3H), 1.43 (d, 3H), 1.37 (quintet, 2H), 1.13 (t, 3H), 1.09 (sextet, 2H), 0.77 (t, 3H); MS m/z [M+H]⁺ 495.8.

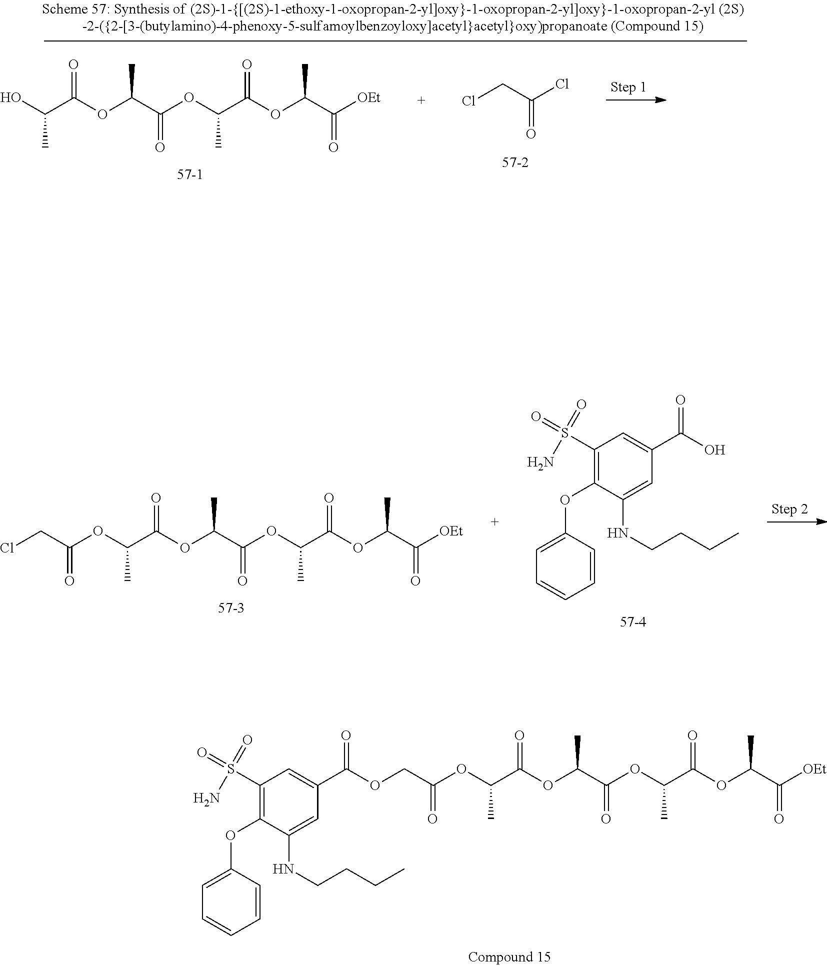 [SMILES: CCOC(=O)[C@H](C)OC(=O)[C@H](C)OC(=O)[C@H](C)OC(=O)[C@H](C)O ; O=C(Cl)CCl ; CCOC(=O)[C@H](C)OC(=O)[C@H](C)OC(=O)[C@H](C)OC(=O)[C@H](C)OC(=O)CCl ; CCCCNc1cc(C(=O)O)cc(S(N)(=O)=O)c1Oc1ccccc1 ; CCCCNc1cc(C(=O)OCC(=O)O[C@@H](C)C(=O)O[C@@H](C)C(=O)O[C@@H](C)C(=O)O[C@@H](C)C(=O)OCC)cc(S(N)(=O)=O)c1Oc1ccccc1]

Step 1: Preparation of (2S)-1-{[(2S)-1-{[(2S)-1-ethoxy-1-oxopropan-2-yl]oxy}-1-oxopropan-2-yl]oxy}-1-oxopropan-2-yl (2S)-2-[(2-chloroacetyl)oxy] Propanoate (57-3): To a solution of (2S)-1-{[(2S)-1-{[(2S)-1-ethoxy-1-oxopropan-2-yl]oxy}-1-oxopropan-2-yl]oxy}-1-oxopropan-2-yl (2S)-2-hydroxypropanoate 57-1 (8.0 g, 23.95 mmol) in DCM (80 mL) was added triethylamine (12.1 mL, 83.83 mmol), followed by chloroacetyl chloride 57-2 (4.82 mL, 59.8 mmol) drop-wise at 0° C. The reaction mixture was allowed to stir at 25-30° C. over a period of 16 h. The resulting reaction mass was diluted with DCM (400 mL), washed with water (2×300 mL), organic layer was dried over sodium sulfate and concentrated under reduced pressure. The crude product obtained upon evaporation of volatiles was purified through silica gel (60-120 mesh) column (10% ethyl acetate in hexane) to give product 57-3 as colorless wax 8.0 g (80%).

Step 2: Preparation of (2S)-1-{[(2S)-1-{[(2S)-1-ethoxy-1-oxopropan-2-yl]oxy}-1-oxopropan-2-yl]oxy}-1-oxopropan-2-yl (2S)-2-({2-[3-(butylamino)-4-phenoxy-5-sulfamoylbenzoyloxy]acetyl}oxy)propanoate (Compound 15): To a solution of bumetanide 57-4 (2.2 g, 6.03 mmol) in N,N-dimethylformamide (22 mL), were added K₂CO₃ (1.08 g, 7.84 mmol), TBAI (0.222 g, 0.603 mmol) and (2S)-1-{[(2S)-1-{[(2S)-1-ethoxy-1-oxopropan-2-yl]oxy}-1-oxopropan-2-yl]oxy}-1-oxopropan-2-yl (2S)-2-[(2-chloroacetyl)oxy]propanoate 57-3 (3.71 g, 9.05 mmol) at 0-5° C. The reaction mixture was allowed to stir at 25-30° C. for 2 h. The resulting reaction mass was diluted with ethyl acetate (300 mL), washed with water (2×100 mL), dried over sodium sulfate and concentrated under reduced pressure. The crude product obtained upon evaporation of volatiles was purified by silica gel column chromatography (35% ethyl acetate in hexane) to give product Compound 15 as off white low melting solid 2.0 g (45%). ¹H NMR (400 MHz, DMSO-d₆) δ 7.73 (d, J=2 Hz 1H), 7.45-7.39 (m, 3H), 7.27 (t, 2H), 7.02 (t, 1H), 6.85 (dd, 2H), 5.28-5.16 (m, 4H), 5.14-5.01 (m, 3H), 4.17-4.06 (m, 2H), 3.10-3.02 (m, 2H), 1.52-1.45 (m, 9H), 1.43 (d, 3H), 1.37 (quintet, 2H), 1.18 (t, 3H), 1.09 (sextet, 2H), 0.77 (t, 3H); MS m/z [M+H]⁺ 739.9.

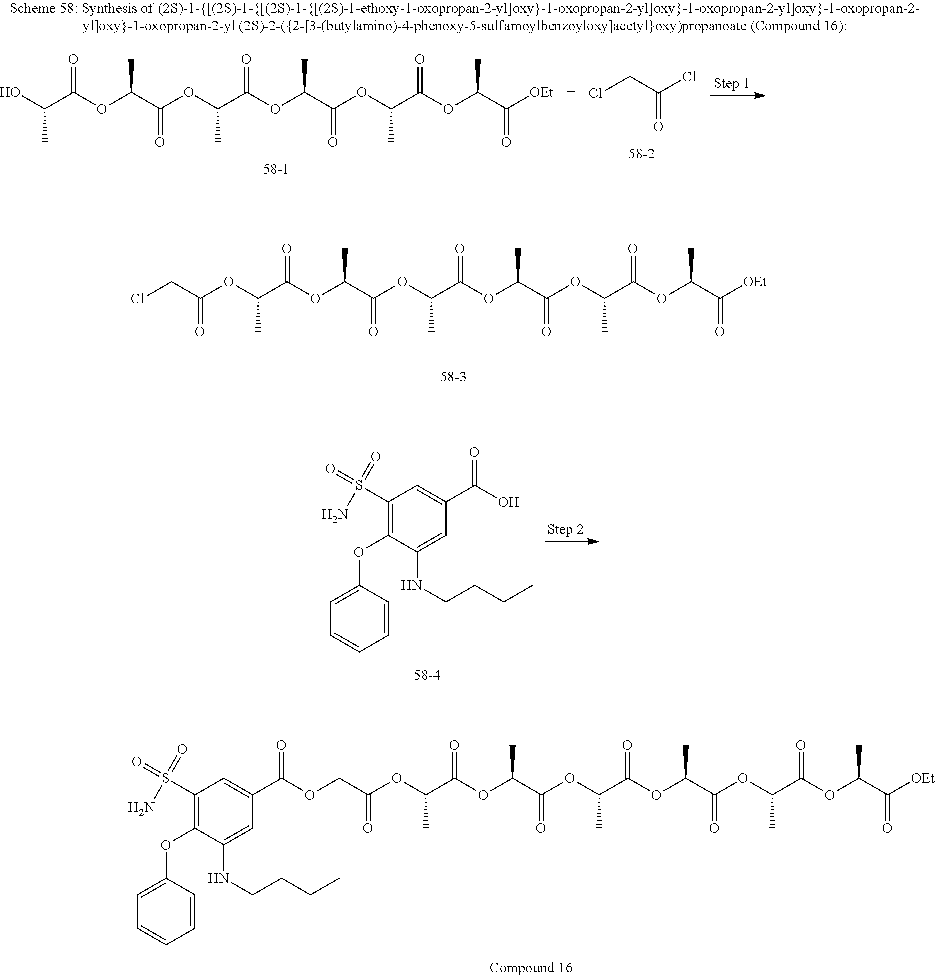 [SMILES: CCOC(=O)[C@H](C)OC(=O)[C@H](C)OC(=O)[C@H](C)OC(=O)[C@H](C)OC(=O)[C@H](C)OC(=O)[C@H](C)O ; O=C(Cl)CCl ; CCOC(=O)[C@H](C)OC(=O)[C@H](C)OC(=O)[C@H](C)OC(=O)[C@H](C)OC(=O)[C@H](C)OC(=O)[C@H](C)OC(=O)CCl ; CCCCNc1cc(C(=O)O)cc(S(N)(=O)=O)c1Oc1ccccc1 ; CCCCNc1cc(C(=O)OCC(=O)O[C@@H](C)C(=O)O[C@@H](C)C(=O)O[C@@H](C)C(=O)O[C@@H](C)C(=O)O[C@@H](C)C(=O)O[C@@H](C)C(=O)OCC)cc(S(N)(=O)=O)c1Oc1ccccc1]

Step 1: Preparation of (2S)-1-{[(2S)-1-{[(2S)-1-{[(2S)-1-{[(2S)-1-ethoxy-1-oxopropan-2-yl]oxy}-1-oxopropan-2-yl]oxy}-1-oxopropan-2-yl]oxy}-1-oxopropan-2-yl]oxy}-1-oxopropan-2-yl (2S)-2-[(2-chloroacetyl)oxy]propanoate (58-3): To a solution of (2S)-1-{[(2S)-1-{[(2S)-1-{[(2S)-1-{[(2S)-1-ethoxy-1-oxopropan-2-yl]oxy}-1-oxopropan-2-yl]oxy}-1-oxopropan-2-yl]oxy}-1-oxopropan-2-yl]oxy}-1-oxopropan-2-yl (2S)-2-hydroxypropanoate 58-1 (10.0 g, 20.9 mmol) in DCM (100 mL), was added triethylamine (15.10 mL, 104.51 mmol) followed by chloroacetyl chloride 58-2 (4.15 mL, 52.25 mmol) drop-wise at 0-5° C. The reaction mixture was allowed to stir at 25-30° C. over a period of 2 h. The resulting reaction mass was diluted with DCM (400 mL), washed with water (2×300 mL), organic layer was dried over sodium sulfate and concentrated under reduced pressure. The crude product obtained upon evaporation of volatiles was purified by silica gel (230-400 mesh) column chromatography (10% ethyl acetate in hexane) to give product 58-3 as colorless wax 8.0 g (69%).

Step 2: Preparation of (2S)-1-{[(2S)-1-{[(2S)-1-{[(2S)-1-{[(2S)-1-ethoxy-1-oxopropan-2-yl]oxy}-1-oxopropan-2-yl]oxy}-1-oxopropan-2-yl]oxy}-1-oxopropan-2-yl]oxy}-1-oxopropan-2-yl (2S)-2-({2-[3-(butylamino)-4-phenoxy-5-sulfamoylbenzoyloxy]acetyl}oxy)propanoate (Compound 16): To a solution of bumetanide 58-4 (2.2 g, 6.03 mmol) in N,N-.dimethylformamide (22 mL), were added K₂CO₃ (1.083 g, 7.84 mmol), TBAI (0.223 g, 0.603 mmol) and (2S)-1-{[(2S)-1-{[(2S)-1-{[(2S)-1-{[(2S)-1-ethoxy-1-oxopropan-2-yl]oxy}-1-oxopropan-2-yl]oxy}-1-oxopropan-2-yl]oxy}-1-oxopropan-2-yl]oxy}-1-oxopropan-2-yl (2S)-2-[(2-chloroacetyl)oxy]propanoate 58-3 (5.025 g, 9.055 mmol) at 0-5° C. The reaction mixture was allowed to stir at 25-30° C. for 2 h. The resulting reaction mass was diluted with ethyl acetate (300 mL), washed with water (2×200 mL), dried over sodium sulfate and concentrated under reduced pressure. The crude product obtained upon evaporation of volatiles was purified by silica gel (230-400 mesh) column chromatography (35% ethyl acetate in hexane) to give product Compound 16 as an off white low melting solid 3.5 g (65%). ¹H NMR (400 MHz, DMSO-d₆) δ 7.73 (d, J=2 Hz 1H), 7.45-7.41 (m, 3H), 7.27 (t, 2H), 7.02 (t, 1H), 6.85 (d, 2H), 5.29-5.15 (m, 6H), 5.13-5.00 (m, 3H), 4.16-4.06 (m, 2H), 3.10-3.02 (m, 2H), 1.52-1.31 (m, 20H), 1.18 (t, 3H), 1.10 (sextet, 2H), 0.77 (t, 3H); MS m/z [M+H]⁺ 884.0.

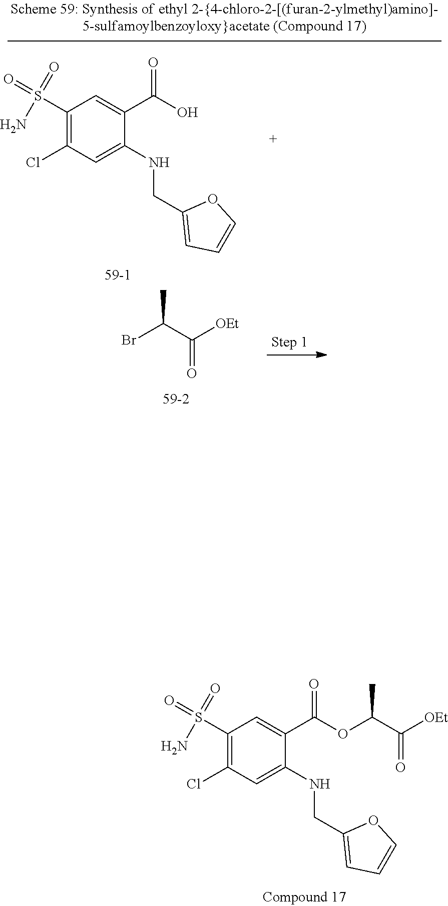

Step 1: Preparation of ethyl 2-{4-chloro-2-[(furan-2-ylmethyl)amino]-5-sulfamoylbenzoyloxy}acetate (Compound 17): To a solution of furosemide 59-1 (5 g, 15 mmol) in N,N-Dimethylformamide (50 mL) were added potassium carbonate (3.13 g, 22 mmol) and ethyl 2-bromoacetate 59-2 (1.5 mL, 13 mmol) at 0-5° C. The reaction mixture was allowed to stir at 25-30° C. over a period of 1 h. The resulting reaction mass was diluted with ethyl acetate (250 mL), washed with water (2×150 mL), dried over sodium sulfate and concentrated under reduced pressure. The crude product obtained upon evaporation of volatiles was stirred with ethanol (20 mL) at 0-5° C., the solid precipitate was collected by filtration and dried under high vacuum to obtain product Compound 17 as a pale yellow solid 4.6 g (73%). ¹H NMR (400 MHz, DMSO-d₆) δ 8.48 (s, 1H), 8.37 (t, 1H), 7.62 (d, J=1 Hz, 1H), 7.40 (s, 2H), 7.14 (s, 1H), 6.45-6.35 (m, 2H), 4.92 (s, 2H), 4.62 (d, J=6 Hz, 2H), 4.17 (q, 2H), 1.21 (t, 3H); MS m/z [M−H]⁻ 415.1.

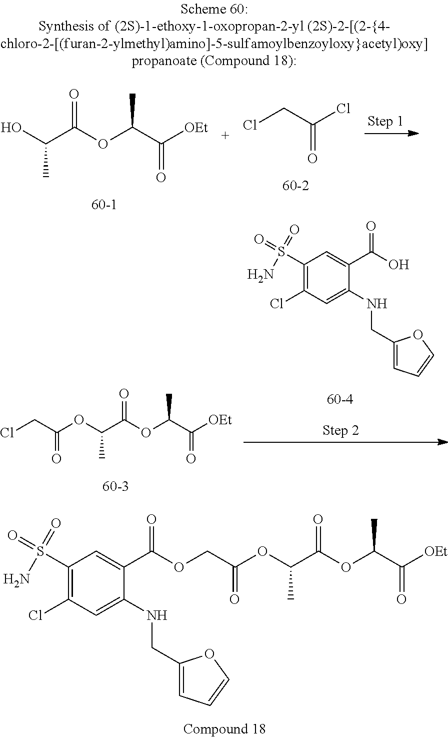 [SMILES: CCOC(=O)[C@H](C)OC(=O)[C@H](C)O ; O=C(Cl)CCl ; NS(=O)(=O)c1cc(C(=O)O)c(NCc2ccco2)cc1Cl ; CCOC(=O)[C@H](C)OC(=O)[C@H](C)OC(=O)CCl ; CCOC(=O)[C@H](C)OC(=O)[C@H](C)OC(=O)COC(=O)c1cc(S(N)(=O)=O)c(Cl)cc1NCc1ccco1]

Step 1: Preparation of (2S)-1-ethoxy-1-oxopropan-2-yl (2S)-2-[(2-chloroacetyl)oxy]propanoate (60-3): To a solution of (2S)-1-ethoxy-1-oxopropan-2-yl (2S)-2-hydroxypropanoate 60-1 (4.0 g, 21.03 mmol) in DCM (40 mL) was added triethylamine (9.10 mL, 63.09 mmol) followed by chloroacetyl chloride 60-2 (2.509 mL, 31.54 mmol) drop-wise at 0° C. The reaction mixture was allowed to stir at 25-30° C. over a period of 16 h. The resulting reaction mass was quenched with water (200 mL), extracted with ethyl acetate (2×250 mL), dried over sodium sulfate and concentrated under reduced pressure. The crude product obtained upon evaporation of volatiles was purified by silica gel (230-400 mesh) column chromatography (8-10% ethyl acetate in hexane) to give product 60-3 as pale yellow oil 4.0 g (71%).

Step 2: Preparation of (2S)-1-ethoxy-1-oxopropan-2-yl (2S)-2-[(2-{4-chloro-2-[(furan-2-ylmethyl)amino]-5-sulfamoylbenzoyloxy}acetyl)oxy]propanoate (Compound 18): To a solution of furosemide 60-4 (3.2 g, 9.67 mmol) in N,N-dimethylformamide (32 mL) were added K₂CO₃ (1.73 g, 12.60 mmol), TBAI (0.357 g, 0.96 mmol) and (2S)-1-ethoxy-1-oxopropan-2-yl (2S)-2-hydroxypropanoate 60-3 (3.86 g, 14.5 mmol) at 0° C. The reaction mixture was allowed to stir at 25-30° C. for 2 h. The resulting reaction mass was diluted with ethyl acetate (300 mL), washed with water (2×200 mL), dried over sodium sulfate and concentrated under reduced pressure. The crude product obtained upon evaporation of volatiles was purified through normal phase column chromatography (25% ethyl acetate in hexane) to give product Compound 18 as a pale yellow solid 1.9 g (35%). ¹H NMR (400 MHz, DMSO-d₆) δ 8.47 (s, 1H), 8.35 (t, 1H), 7.62 (dd, 1H), 7.40 (s, 2H), 7.14 (s, 1H), 6.43-6.34 (m, 2H), 5.21 (q, 1H), 5.12-4.98 (m, 3H), 4.62 (d, 2H), 4.17-4.07 (m, 2H), 1.48 (d, 3H), 1.42 (d, 3H), 1.18 (t, 3H); MS m/z [M+H]⁺ 562.1.

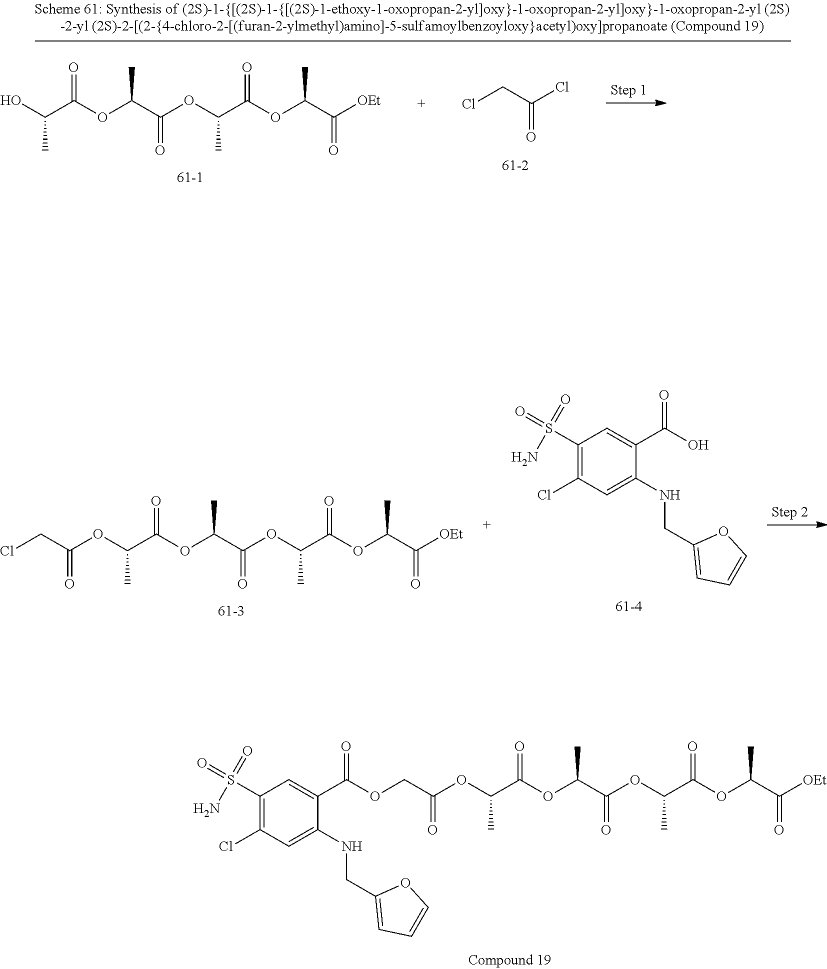 [SMILES: CCOC(=O)[C@H](C)OC(=O)[C@H](C)OC(=O)[C@H](C)OC(=O)[C@H](C)O ; O=C(Cl)CCl ; CCOC(=O)[C@H](C)OC(=O)[C@H](C)OC(=O)[C@H](C)OC(=O)[C@H](C)OC(=O)CCl ; NS(=O)(=O)c1cc(C(=O)O)c(NCc2ccco2)cc1Cl ; CCOC(=O)[C@H](C)OC(=O)[C@H](C)OC(=O)[C@H](C)OC(=O)[C@H](C)OC(=O)COC(=O)c1cc(S(N)(=O)=O)c(Cl)cc1NCc1ccco1]

Step 1: Preparation of (2S)-1-{[(2S)-1-{[(2S)-1-ethoxy-1-oxopropan-2-yl]oxy}-1-oxopropan-2-yl]oxy}-1-oxopropan-2-yl(2S)-2-[(2-chloroacetyl)oxy]propanoate (61-3): To a solution of (2S)-1-{[(2S)-1-{[(2S)-1-ethoxy-1-oxopropan-2-yl]oxy}-1-oxopropan-2-yl]oxy}-1-oxopropan-2-yl (2S)-2-hydroxypropanoate 61-1 (3.0 g, 8.97 mmol) in DCM (30 mL) was added triethylamine (6.47 mL, 44.80 mmol) followed by chloroacetyl chloride 61-2 (1.78 mL, 22.4 mmol) drop-wise at 0° C. The reaction mixture was allowed to stir at 25-30° C. over a period of 16 h. The resulting reaction mass was diluted with DCM (200 mL), washed with water (2×150 mL), organic layer was dried over sodium sulfate and concentrated under reduced pressure. The crude product obtained upon evaporation of volatiles was purified by silica gel (60-120 mesh) column chromatography (10% ethyl acetate in hexane) to give product 61-3 as colorless wax 2.9 g (78%).

Step 2: Preparation of (2S)-1-{[(2S)-1-{[(2S)-1-ethoxy-1-oxopropan-2-yl]oxy}-1-oxopropan-2-yl]oxy}-1-oxopropan-2-yl (2S)-2-[(2-{4-chloro-2-[(furan-2-ylmethyl)amino]-5-sulfamoylbenzoyloxy}acetyl)oxy]propanoate (Compound 19): To a solution of furosemide 61-4 (1.4 g, 4.23 mmol) in N,N-dimethylformamide (14 mL) were added K₂CO₃ (0.701 g, 5.08 mmol), TBAI (0.156 g, 0.423 mmol) and (2S)-1-{[(2S)-1-{[(2S)-1-ethoxy-1-oxopropan-2-yl]oxy}-1-oxopropan-2-yl]oxy}-1-oxopropan-2-yl (2S)-2-[(2-chloroacetyl)oxy]propanoate 61-3 (2.43 g, 5.92 mmol) at 0-5° C. The reaction mixture was allowed to stir at 25-30° C. over a period of 16 h. The resulting reaction mass was diluted with ethyl acetate (200 mL), washed with water (2×150 mL), dried over sodium sulfate and concentrated under reduced pressure. The crude product obtained upon evaporation of volatiles was purified through normal phase column chromatography (30% ethyl acetate in hexane) to give product Compound 19 as a pale yellow low melting solid 1.8 g (60%). ¹H NMR (400 MHz, DMSO-d₆) δ 8.47 (s, 1H), 8.35 (t, 1H), 7.62 (dd, 1H), 7.40 (s, 2H), 7.14 (s, 1H), 6.43-6.35 (m, 2H), 5.26-5.15 (m, 3H), 5.12-4.99 (m, 3H), 4.62 (d, 2H), 4.17-4.08 (m, 2H), 1.50-1.39 (m, 12H), 1.18 (t, 3H); MS m/z [M+H]⁺ 706.4.

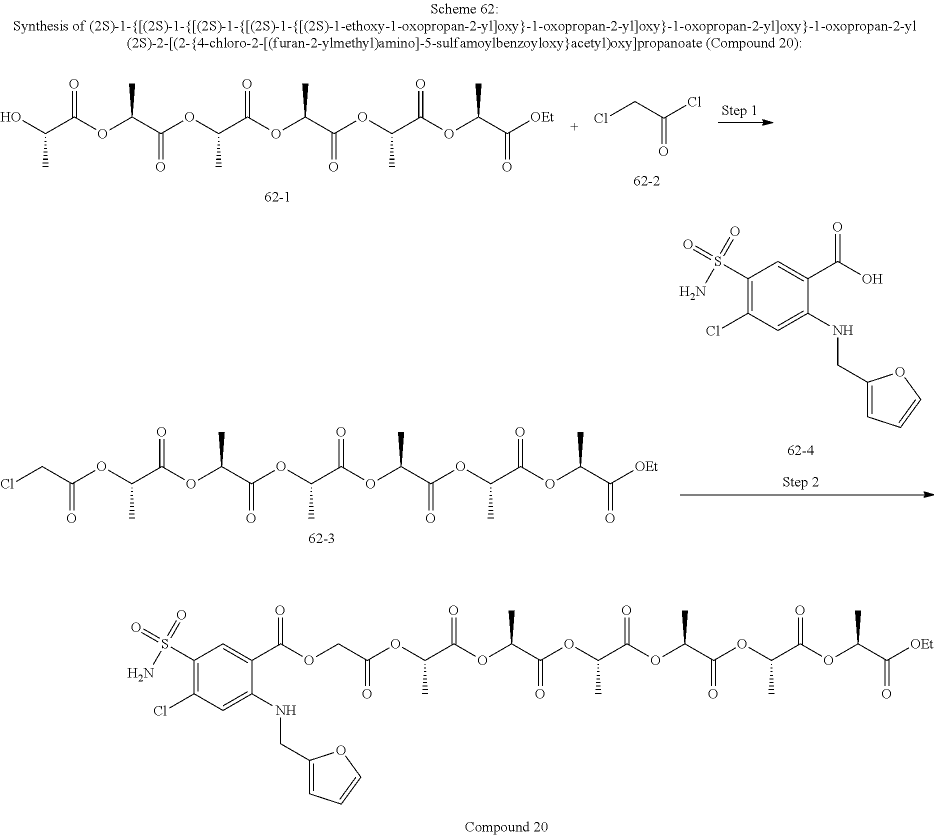 [SMILES: CCOC(=O)[C@H](C)OC(=O)[C@H](C)OC(=O)[C@H](C)OC(=O)[C@H](C)OC(=O)[C@H](C)OC(=O)[C@H](C)O ; O=C(Cl)CCl ; NS(=O)(=O)c1cc(C(=O)O)c(NCc2ccco2)cc1Cl ; CCOC(=O)[C@H](C)OC(=O)[C@H](C)OC(=O)[C@H](C)OC(=O)[C@H](C)OC(=O)[C@H](C)OC(=O)[C@H](C)OC(=O)CCl ; CCOC(=O)[C@H](C)OC(=O)[C@H](C)OC(=O)[C@H](C)OC(=O)[C@H](C)OC(=O)[C@H](C)OC(=O)[C@H](C)OC(=O)COC(=O)c1cc(S(N)(=O)=O)c(Cl)cc1NCc1ccco1]

Step 1: Preparation of (2S)-1-{[(2S)-1-{[(2S)-1-{[(2S)-1-{[(2S)-1-ethoxy-1-oxopropan-2-yl]oxy}-1-oxopropan-2-yl]oxy}-1-oxopropan-2-yl]oxy}-1-oxopropan-2-yl]oxy}-1-oxopropan-2-yl (2S)-2-[(2-chloroacetyl)oxy] propanoate (62-3): To a solution of (2S)-1-{[(2S)-1-{[(2S)-1-{[(2S)-1-{[(2S)-1-ethoxy-1-oxopropan-2-yl]oxy}-1-oxopropan-2-yl]oxy}-1-oxopropan-2-yl]oxy}-1-oxopropan-2-yl]oxy}-1-oxopropan-2-yl (2S)-2-hydroxypropanoate 62-1 (10.0 g, 20.9 mmol) in DCM (100 mL) was added triethylamine (15.10 mL, 104.51 mmol) followed by chloroacetyl chloride 62-2 (4.15 mL, 52.25 mmol) drop-wise at 0° C. The reaction mixture was allowed to stir at 25-30° C. over a period of 2 h. The resulting reaction mass was diluted with DCM (400 mL), washed with water (2×300 mL), organic layer was dried over sodium sulfate and concentrated under reduced pressure. The crude product obtained upon evaporation of volatiles was purified by silica gel (230-400 mesh) column chromatography (10% ethyl acetate in hexane) to give product 62-3 as colorless wax 8.0 g (69%).

Step 2: Preparation of (2S)-1-{[(2S)-1-{[(2S)-1-{[(2S)-1-{[(2S)-1-ethoxy-1-oxopropan-2-yl]oxy}-1-oxopropan-2-yl]oxy}-1-oxopropan-2-yl]oxy}-1-oxopropan-2-yl]oxy}-1-oxopropan-2-yl (2S)-2-[(2-{4-chloro-2-[(furan-2-ylmethyl)amino]-5-sulfamoylbenzoyloxy}acetyl)oxy]propanoate (Compound 20): To a solution of furosemide 62-4 (1.2 g, 3.64 mmol) in N,N-dimethylformamide (12 mL) were added K₂CO₃ (0.6 g, 4.37 mmol), TBAI (0.13 g, 0.36 mmol) and (2S)-1-{[(2S)-1-{[(2S)-1-{[(2S)-1-{[(2S)-1-ethoxy-1-oxopropan-2-yl]oxy}-1-oxopropan-2-yl]oxy}-1-oxopropan-2-yl]oxy}-1-oxopropan-2-yl]oxy}-1-oxopropan-2-yl (2S)-2-[(2-chloroacetyl)oxy]propanoate 62-3 (2.83 g, 5.0 mmol) at 0-5° C. The reaction mixture was allowed to stir at 25-30° C. for 2 h. The resulting reaction mass was diluted with ethyl acetate (300 mL), washed with water (2×200 mL), dried over sodium sulfate and concentrated under reduced pressure. The crude product obtained upon evaporation of volatiles was purified through normal phase grace column chromatography (37% ethyl acetate in hexane) to give product Compound 20 as a pale yellow wax 1.6 g (51%). ¹H NMR (400 MHz, DMSO-d₆) δ 8.47 (s, 1H), 8.34 (t, 1H), 7.62 (d, 1H), 7.40 (s, 2H), 7.14 (s, 1H), 6.43-6.35 (m, 2H), 5.27-5.15 (m, 5H), 5.13-4.99 (m, 3H), 4.62 (d, 2H), 4.17-4.08 (m, 2H), 1.50-1.39 (m, 18H), 1.18 (t, 3H); MS m/z [M+NH₄]⁺ 867.1.

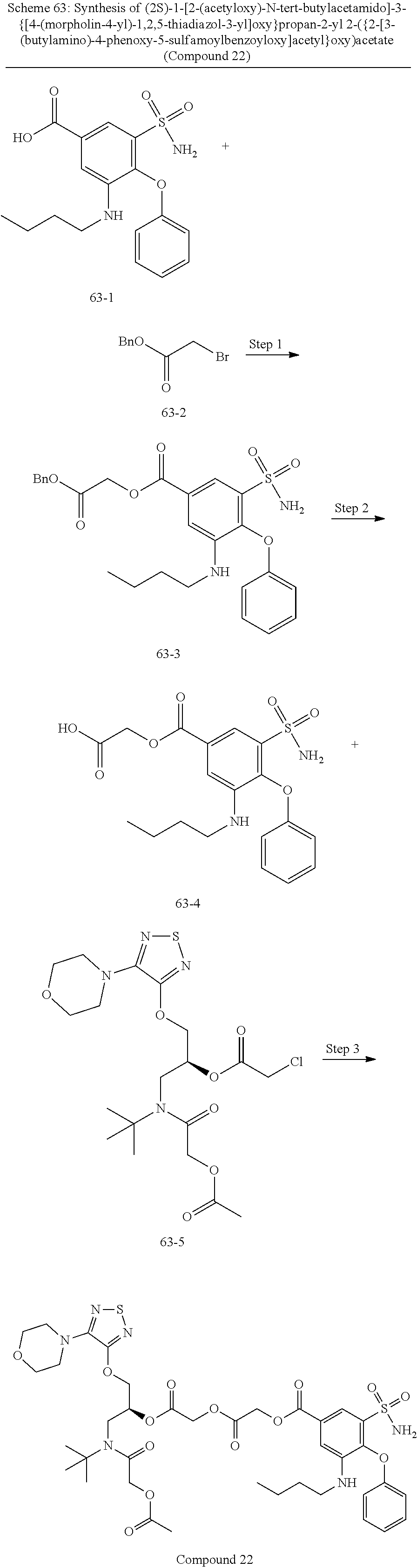 [SMILES: CCCCNc1cc(C(=O)O)cc(S(N)(=O)=O)c1Oc1ccccc1 ; O=C(CBr)OCc1ccccc1 ; CCCCNc1cc(C(=O)OCC(=O)OCc2ccccc2)cc(S(N)(=O)=O)c1Oc1ccccc1 ; CCCCNc1cc(C(=O)OCC(=O)O)cc(S(N)(=O)=O)c1Oc1ccccc1 ; CC(=O)OCC(=O)N(C[C@@H](COc1nsnc1N1CCOCC1)OC(=O)CCl)C(C)(C)C ; CCCCNc1cc(C(=O)OCC(=O)OCC(=O)O[C@H](COc2nsnc2N2CCOCC2)CN(C(=O)COC(C)=O)C(C)(C)C)cc(S(N)(=O)=O)c1Oc1ccccc1]

Step 1: Preparation of benzyl 2-[3-(butylamino)-4-phenoxy-5-sulfamoylbenzoyloxy]acetate (63-3): To a solution of bumetanide 63-1 (2.0 g, 5.48 mmol) in N,N-Dimethylformamide (20 mL) were added potassium carbonate (1.136 g, 8.23 mmol) and benzyl 2-bromoacetate 63-2 (0.698 mL, 4.39 mmol) at 0° C. The reaction mixture was allowed to stir at 25-30° C. over a period of 2 h. The resulting reaction mass was diluted with ethyl acetate (250 mL), washed with water (2×150 mL), organic layer was dried over sodium sulfate and concentrated under reduced pressure. The crude product obtained upon evaporation of volatiles was purified through silica gel (230-400 mesh) column chromatography to obtain product 63-3 as an off white solid 2.0 g (71%).

Step 2: Preparation of 2-[3-(butylamino)-4-phenoxy-5-sulfamoylbenzoyloxy]acetic acid (63-4): To a 100 mL autoclave vessel were added a solution of benzyl 2-[3-(butylamino)-4-phenoxy-5-sulfamoylbenzoyloxy]acetate 63-3 (2.0 g, 3.90 mmol) in methanol (20 mL) and 10% Pd/C (400 mg, 50% wet) at 25-30° C. The reaction mixture was stirred at 25-30° C. under hydrogen pressure (5 kg/cm²) over a period of 1 h. After completion of the reaction, the reaction mixture was filtered through celite bed. Then volatiles were evaporated under reduced pressure to obtain crude compound. The crude compound was stirred with diethyl ether (20 mL) at 0-5° C. The solid precipitate obtained was filtered and dried under high vacuum to afford 63-4 as an off white solid 1.4 g (85%).

Step 3: Preparation of (2S)-1-[2-(acetyloxy)-N-tert-butylacetamido]-3-{[4-(morpholin-4-yl)-1,2,5-thiadiazol-3-yl]oxy}propan-2-yl 2-({2-[3-(butylamino)-4-phenoxy-5-sulfamoylbenzoyloxy]acetyl}oxy)acetate (Compound 22): To a solution of 2-[3-(butylamino)-4-phenoxy-5-sulfamoylbenzoyloxy]acetic acid 63-4 (1.0 g, 2.36 mmol) in N,N-Dimethylformamide (10 mL) were added potassium carbonate (0.392 g, 2.84 mmol), TBAI (87 mg, 0.236 mmol) and (2S)-1-[2-(acetyloxy)-N-tert-butylacetamido]-3-{[4-(morpholin-4-yl)-1,2,5-thiadiazol-3-yl]oxy}propan-2-yl 2-chloroacetate 63-5 (1.63 g, 3.31 mmol) at 0-5° C. The reaction mixture was allowed to stir at 25-30° C. over a period of 2 h. The resulting reaction mass was diluted with ethyl acetate (250 mL), washed with water (2×150 mL), organic layer was dried over sodium sulfate and concentrated under reduced pressure. The crude product obtained upon evaporation of volatiles was purified by silica gel (230-400 mesh) column chromatography to obtain product Compound 22 as a white solid 1.2 g (57%). ¹H-NMR (400 MHz, DMSO-d6) δ 7.73 (d, J=2 Hz 1H), 7.46-7.37 (m, 3H), 7.27 (t, 2H), 7.02 (t, 1H), 6.85 (d, 2H), 5.53-5.43 (m, 1H), 5.22 (t, 1H), 5.13-5.03 (m, 2H), 4.97-4.82 (m, 3H), 4.70-4.55 (m, 2H), 4.50-4.42 (m, 1H), 3.71-3.53 (m, 6H), 3.43-3.3 (m, 4H), 3.07 (q, 2H), 2.08 (s, 3H), 1.41-1.25 (m, 11H), 1.14-1.03 (m, 2H), 0.76 (t, 3H). MS m/z [M+H]⁺ 880.3.

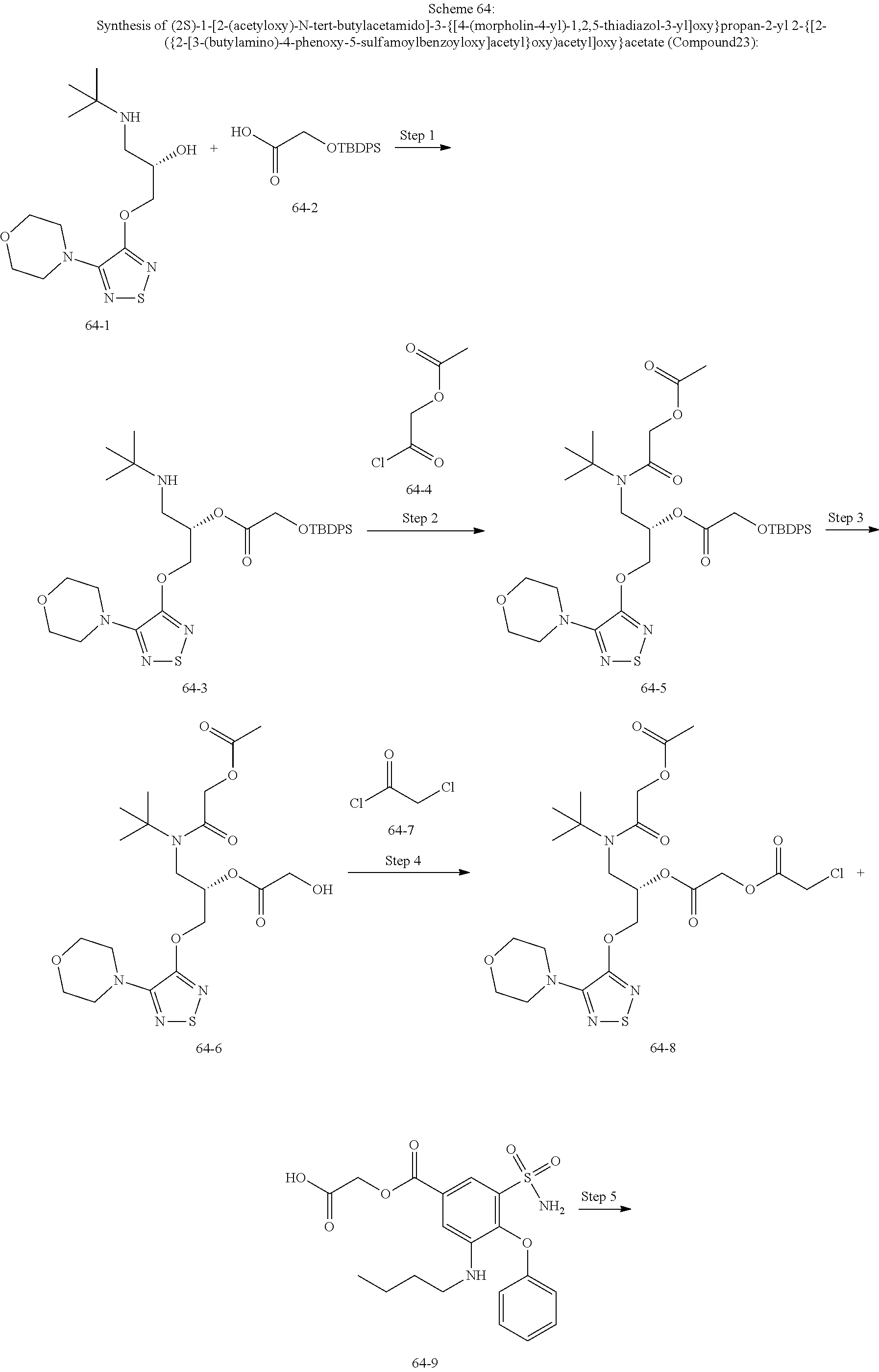

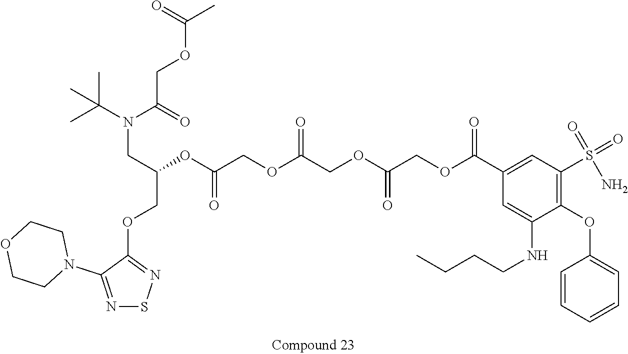 [SMILES: CCCCNc1cc(C(=O)OCC(=O)OCC(=O)OCC(=O)O[C@H](COc2nsnc2N2CCOCC2)CN(C(=O)COC(C)=O)C(C)(C)C)cc(S(N)(=O)=O)c1Oc1ccccc1]

Step 1: Preparation of (2S)-1-(tert-butylamino)-3-{[4-(morpholin-4-yl)-1,2,5-thiadiazol-3-yl]oxy}propan-2-yl 2-[(tert-butyldiphenylsilyl)oxy]acetate (64-3): To a solution of 2-[(tert-butyldiphenylsilyl)oxy]acetic acid 64-2 (12.90 g, 41.08 mmol) in DCM (100 mL) were added EDC.HCl (9.05 g, 47.4 mmol), timolol 64-1 (10.0 g, 31.60 mmol) and 4-Dimethylaminopyridine (0.385 g, 3.16 mmol) at 0-5° C. The reaction mixture was allowed to stir at 25-30° C. over a period of 1 h. The resulting reaction mass was diluted with ethyl acetate (200 mL), washed with water (2×150 mL), dried over sodium sulfate and concentrated under reduced pressure to give crude product 64-3 as a colorless wax 16.0 g. The obtained compound was taken forward to next step without any further purification.

Step 2: Preparation of (2S)-1-[2-(acetyloxy)-N-tert-butylacetamido]-3-{[4-(morpholin-4-yl)-1,2,5-thiadiazol-3-yl]oxy}propan-2-yl 2-[(tert-butyldiphenylsilyl)oxy]acetate (64-5): To a solution of (2S)-1-(tert-butylamino)-3-{[4-(morpholin-4-yl)-1,2,5-thiadiazol-3-yl]oxy}propan-2-yl 2-[(tert-butyldiphenylsilyl)oxy]acetate 64-3 (16.0 g, 26.10 mmol) in Chloroform (160 mL) was added triethylamine (7.54 mL, 52.21 mmol), followed by acetoxy acetyl chloride 64-4 (4.20 mL, 39.16 mmol) drop-wise at 0° C. The reaction mixture was allowed to stir at 25-30° C. over a period of 2 h. The resulting reaction mass was diluted with DCM (400 mL), washed with water (2×300 mL), organic layer was dried over sodium sulfate and concentrated under reduced pressure. The crude product obtained upon evaporation of volatiles was purified through silica gel (230-400 mesh) column chromatography to give product 64-5 as a colorless wax 12.0 g (64%).

Step 3: Preparation of (2S)-1-[2-(acetyloxy)-N-tert-butylacetamido]-3-{[4-(morpholin-4-yl)-1,2,5-thiadiazol-3-yl]oxy}propan-2-yl 2-hydroxyacetate (64-6): To a solution of (2S)-1-[2-(acetyloxy)-N-tert-butylacetamido]-3-{[4-(morpholin-4-yl)-1,2,5-thiadiazol-3-yl]oxy}propan-2-yl2-[(tert-butyldiphenylsilyl)oxy]acetate 64-5 (6.0 g, 8.42 mmol) in tetrahydrofuran (60 mL) were added tetra-n-butylammonium fluoride (4.21 mL, 1.0 M, 4.21 mmol) and acetic acid (0.229 mL, 4.21 mmol) at 0° C. The reaction mixture was allowed to stir at 0° C. to 25-30° C. over a period of 45 minutes. The resulting reaction mixture was concentrated under reduced pressure and crude product obtained upon evaporation of the volatiles was purified through silica gel column chromatography to give product 64-6 as a colorless wax 2.9 g (72%).

Step 4: Preparation of (2S)-1-[2-(acetyloxy)-N-tert-butylacetamido]-3-{[4-(morpholin-4-yl)-1,2,5-thiadiazol-3-yl]oxy}propan-2-yl 2-[(2-chloroacetyl)oxy]acetate (64-8): To a solution of (2S)-1-[2-(acetyloxy)-N-tert-butylacetamido]-3-{[4-(morpholin-4-yl)-1,2,5-thiadiazol-3-yl]oxy}propan-2-yl 2-hydroxyacetate 64-6 (2.9 g, 6.11 mmol) in DCM (29 mL) was added triethylamine (2.65 mL, 18.33 mmol), followed by chloroacetyl chloride 64-7 (0.729 mL, 9.16 mmol) drop-wise at 0° C. The reaction mixture was allowed to stir at 0° C. to 25-30° C. over a period of 2 h. The resulting reaction mass was diluted with DCM (250 mL), washed with water (2×150 mL), organic layer was dried over sodium sulfate and concentrated under reduced pressure. The crude product obtained upon evaporation of volatiles was purified through silica gel (230-400 mesh) column chromatography to give product 64-8 as a colorless wax 2.4 g (86%).

Step 5: Preparation of (2S)-1-[2-(acetyloxy)-N-tert-butylacetamido]-3-{[4-(morpholin-4-yl)-1,2,5-thiadiazol-3-yl]oxy}propan-2-yl 2-{[2-({2-[3-(butylamino)-4-phenoxy-5-sulfamoylbenzoyloxy]acetyl}oxy)acetyl]oxy}acetate (Compound 23): To a solution of 2-[3-(butylamino)-4-phenoxy-5-sulfamoylbenzoyloxy]acetic acid 64-9 (2.4 g, 5.68 mmol) in N,N-Dimethylformamide (12 mL) were added potassium carbonate (1.568 g, 11.36 mmol), TBAI (0.20 g, 0.568 mmol) and (2S)-1-[2-(acetyloxy)-N-tert-butylacetamido]-3-{[4-(morpholin-4-yl)-1,2,5-thiadiazol-3-yl]oxy}propan-2-yl2-[(2-chloroacetyl)oxy]acetate 64-8 (3.44 g, 6.24 mmol) at 0° C. The reaction mixture was allowed to stir at 25-30° C. over a period of 2 h. The resulting reaction mass was diluted with ethyl acetate (250 mL), washed with water (2×150 mL), organic layer was dried over sodium sulfate and concentrated under reduced pressure. The crude product obtained upon evaporation of volatiles was purified through silica gel (230-400 mesh) column chromatography to give product Compound 23 as an off white solid 2.2 g (86%). ¹H-NMR (400 MHz, DMSO-d6) δ 7.75 (d, J=2 Hz 1H), 7.46-7.39 (m, 3H), 7.27 (t, 2H), 7.02 (t, 1H), 6.85 (d, 2H), 5.53-5.36 (m, 1H), 5.22 (t, 1H), 5.08 (s, 2H), 4.98-4.80 (m, 5H), 4.7-4.55 (m, 2H), 4.50-4.42 (m, 1H), 3.71-3.52 (m, 6H), 3.45-3.3 (m, 4H), 3.06 (q, 2H), 2.09 (s, 3H), 1.41-1.25 (m, 11H), 1.14-1.03 (m, 2H), 0.77 (t, 3H). MS m/z [M+H]⁺ 938.4.

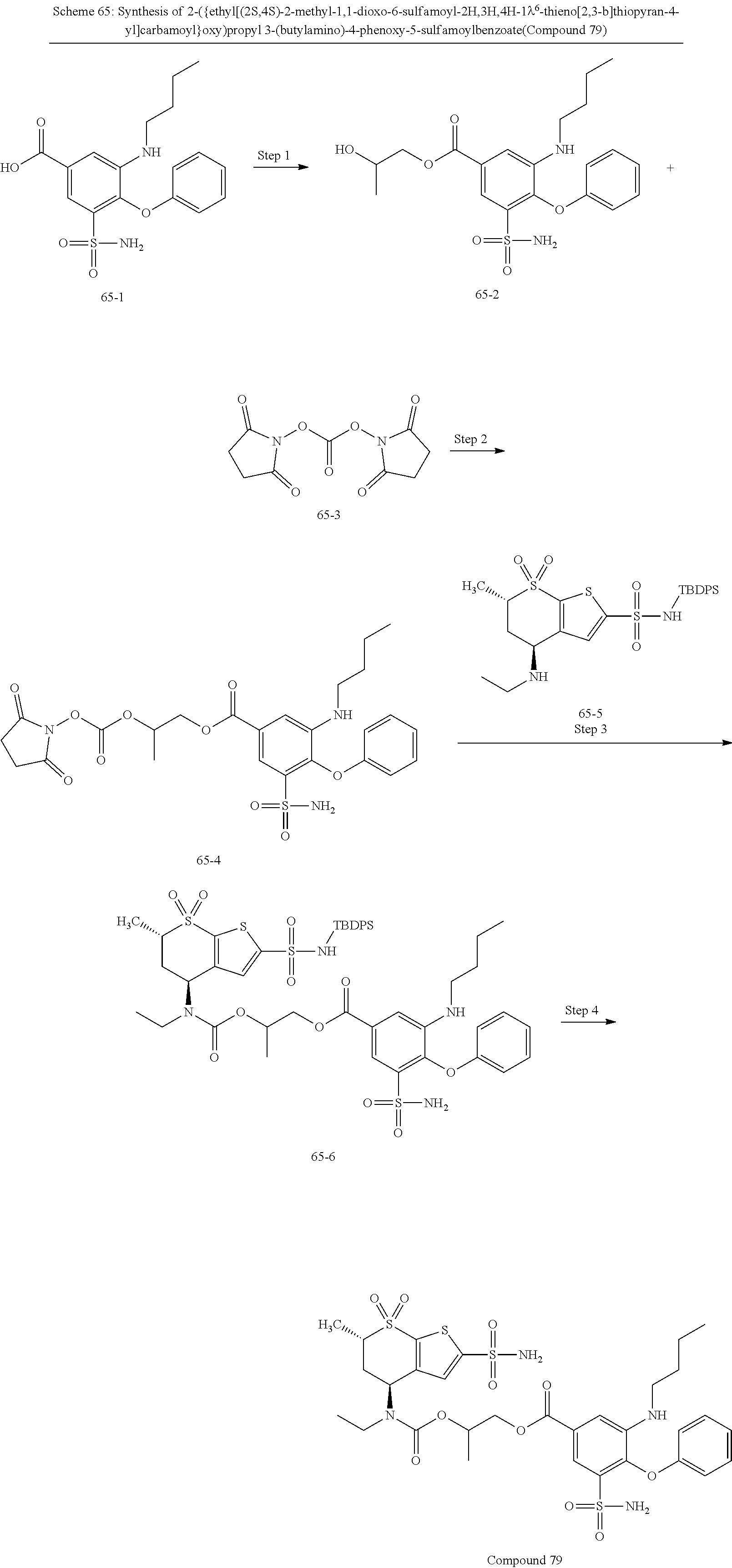

Step 1: Preparation of 2-hydroxypropyl 3-(butylamino)-4-phenoxy-5-sulfamoylbenzoate (65-2): To a solution of bumetanide 65-1 (5.0 g, 13.73 mmol) in THF (50 mL) were added EDC.HCl (3.9 g, 20.5 mmol), HOBt (5.2 g, 13.7 mmol), propylene glycol (1.35 g, 17.8 mmol) and 4-Dimethylaminopyridine (0.3 g, 2.74 mmol) at 0-5° C. The reaction mixture was refluxed at 80° C. for 16 h. The resulting reaction mixture was diluted with ethyl acetate (300 mL) and washed with water (2×150 mL). The organic layer was dried over sodium sulfate and concentrated under reduced pressure at 45° C. The crude compound was purified by reverse phase column chromatography to obtain product 65-2 as white solid 2.5 g (43%).

Step 2: Preparation of 2-({[(2,5-dioxopyrrolidin-1-yl)oxy]carbonyl}oxy)propyl 3-(butylamino)-4-phenoxy-5-sulfamoylbenzoate (65-4): To a solution of 2-hydroxypropyl 3-(butylamino)-4-phenoxy-5-sulfamoylbenzoate 65-2 (1 g, 2.36 mmol) in tetrahydrofuran (10 mL) was added Pyridine (0.8 mL, 8.26 mmol), bis(2,5-dioxopyrrolidin-1-yl) carbonate 65-3 (1.8, 7.10 mmol) and 4-Dimethylaminopyridine (0.057 g, 0.47 mmol) at 0° C. The reaction mixture was stirred at 25-30° C. over a period of 16 h. The resulting reaction mixture was diluted with ethyl acetate (300 mL) and washed with water (2×150 mL). The organic layer was dried over sodium sulfate and concentrated under reduced pressure. The crude product obtained upon evaporation of volatiles was recrystallized using methanol to obtain product 65-4 as a white solid 1 g (76%).

Step 3: Preparation of 2-({[(2S,4S)-6-[(tert-butyldiphenylsilyl)sulfamoyl]-2-methyl-1,1-dioxo-2H,3H,4H-1λ⁶-thieno[2,3-b]thiopyran-4-yl](ethyl)carbamoyl}oxy)propyl 3-(butylamino)-4-phenoxy-5-sulfamoylbenzoate (65-6): To a solution of (2S,4S)—N-(tert-butyldiphenylsilyl)-4-(ethylamino)-2-methyl-1,1-dioxo-2H,3H,4H-1λ⁶-thieno[2,3-b]thiopyran-6-sulfonamide 65-5 (0.3 g, 0.533 mmol) in THF (50 mL) was added Pyridine (0.1 mL, 1.06 mmol), 2-({[(2,5-dioxopyrrolidin-1-yl)oxy]carbonyl}oxy)propyl3-(butylamino)-4-phenoxy-5sulfamoylbenzoate 65-4 (0.3 g, 0.53 mmol) and 4-Dimethylaminopyridine (0.013 g, 0.10 mmol) at 0-5° C. The reaction mixture was stirred at 80° C. over a period of 24 h. The resulting reaction mixture was diluted with ethyl acetate (300 mL) and washed with water (2×150 mL). The organic layer was dried over sodium sulfate and concentrated under reduced pressure to afford 65-6 as a white solid 0.4 g (crude compound 65-6 was taken as such into next step without any purification).

Step 4: Preparation of 2-({ethyl[(2S,4S)-2-methyl-1,1-dioxo-6-sulfamoyl-2H,3H,4H-1λ⁶-thieno[2,3-b]thiopyran-4-yl]carbamoyl}oxy)propyl 3-(butylamino)-4-phenoxy-5-sulfamoylbenzoate (Compound 79): To a solution of 2-({[(2S,4S)-6-[(tert-butyldiphenylsilyl)sulfamoyl]-2-methyl-1,1-dioxo-2H,3H,4H-1λ⁶-thieno[2,3-b]thiopyran-4-yl](ethyl)carbamoyl}oxy)propyl 3-(butylamino)-4-phenoxy-5-sulfamoylbenzoate 65-6 (0.4 g, 0.39 mmol) in tetrahydrofuran (5 mL) were added tetra butyl ammonium fluoride (0.11 mL, 1M, 0.11 mmol) and acetic acid (0.006 mL, 0.11 mmol) at 0-5° C. The reaction mixture was allowed to stir at 0-5° C. for 30 min. The resulting reaction mixture was diluted with ethyl acetate (200 mL) and washed with water (2×100 mL). The organic layer was dried over sodium sulfate and concentrated under reduced pressure. The crude product obtained upon evaporation of volatiles was purified by preparative HPLC to give product Compound 79 as a white solid 90 mg (30%).

¹H NMR (400 MHz, DMSO-d₆) δ 8.48 (s, 1H), 8.1-7.3 (m, 6H), 7.26 (t, J=8 Hz, 2H), 7.01 (t, J=8 Hz, 1H), 6.84 (d, J=8 Hz, 2H), 5.24-4.91 (m, 3H), 4.7-4.0 (m, 2H), 3.89-3.75 (m, 1H), 3.5-3.0 (m, 4H), 2.81-2.70 (m, 1H), 2.45-2.30 (m, 1H), 1.40-1.21 (m, 8H), 1.18-1.00 (m, 5H), 0.76 (t, 3H); MS m/z [M+H]⁺ 773.3.

### Example 14. Analyses of Prodrugs of the Loop Diuretics Furosemide and Bumetanide

HPLC Method for Analysis of Furosemide, Bumetanide and their PLA Conjugates

Chromatographic separation of prodrug, intermediates and parent drug was achieved using an Agilent 1260 Infinity HPLC equipped with a diode array and a multiple wavelength detector with an XTERRA C8 column (5 μm, 4.6 mm×150 mm) as the stationary phase. The mobile phase consisted of an acetonitrile/water gradient as illustrated in Table 1. The mobile phase was stabilized with 0.1% formic acid. The flow rate was 1.0 mL/min, the detection wavelength was 230 nm, and the injection volume was 10 μL. Column temperature was 25° C.

TABLE 1
HPLC gradient for separation of prodrugs of bumetanide and furosemide

| Time (min) | A (water + 0.1% FA) | B (MeCN + 0.1% FA) |
|---|---|---|
| 0 | 95 | 5 |
| 4 | 5 | 95 |
| 5 | 5 | 95 |
| 5.5 | 95 | 5 |
| 7 | 95 | 5 |

### Example 15. Determination of Prodrug Solubility

For each test, approximately 5-10 mg was transferred to a 10 mL glass vial. Aqueous or organic solvent was added to each vial to achieve an overall concentration of 50 mg/mL. After vortexing aggressively for 2-3 minutes and sonicating in a bath sonicator for 5 minutes, undissolved drug was spun down at 1200×g for 5 minutes to generate a pellet. The supernatant was collected and filtered through a 0.2 μm nylon syringe filter into HPLC vials for drug content analysis. Drug concentration was determined by comparing against a standard calibration curve.

### Solubility of Compounds Containing Bumetanide and Furosemide

Microencapsulation techniques use utilize organic solvent to dissolve the polymer and drug. Thus, it is necessary to determine the drug solubility which will inform on the feasibility of microencapsulation. As shown in Table 2, native drug and prodrugs of bumetanide and furosemide are relatively insoluble in water (<0.1 mg/mL) and highly soluble in DMSO and dichloromethane.

TABLE 2
Solubility of prodrugs of bumetanide and furosemide

| | | Compd | Solubility (mg/mL) | | |
|---|---|---|---|---|---|
| | Compound Name | Code | Water | DMSO | DCM |
| Bumetanide Parent | Bumetanide | — | <0.1 | 25 | 25 |
| PLA-Bumetanide | Bumetanide-ethyl- | Compd 2 | <0.1 | >100 | >50 |
| Prodrug | PLA (n = 4) | | | | |
| Furosemide Parent | Furosemide | — | <0.1 | 30 | 25 |
| PLA-Furosemide | Furosemide-ethyl- | Compd 1 | <0.1 | >100 | >50 |
| Prodrugs | PLA (n = 4) | | | | |
| | Furosemide-ethyl- | Compd 5 | <0.1 | >100 | >50 |
| | PLA (n = 6 | | | | |

### Example 16. Determination of Prodrug Stability

### In Vitro Stability of Prodrugs of Bumetanide and Furosemide

Prodrugs of bumetanide and furosemide were solubilized with the addition of 25% (v/v) DMSO and subsequently diluted with water to a concentration of 0.1 mg/mL. The samples were incubated at 37° C. and at various time points, aliquots were collected, filtered through a 0.2 μm nylon syringe filter, and analyzed by RP-HPLC.

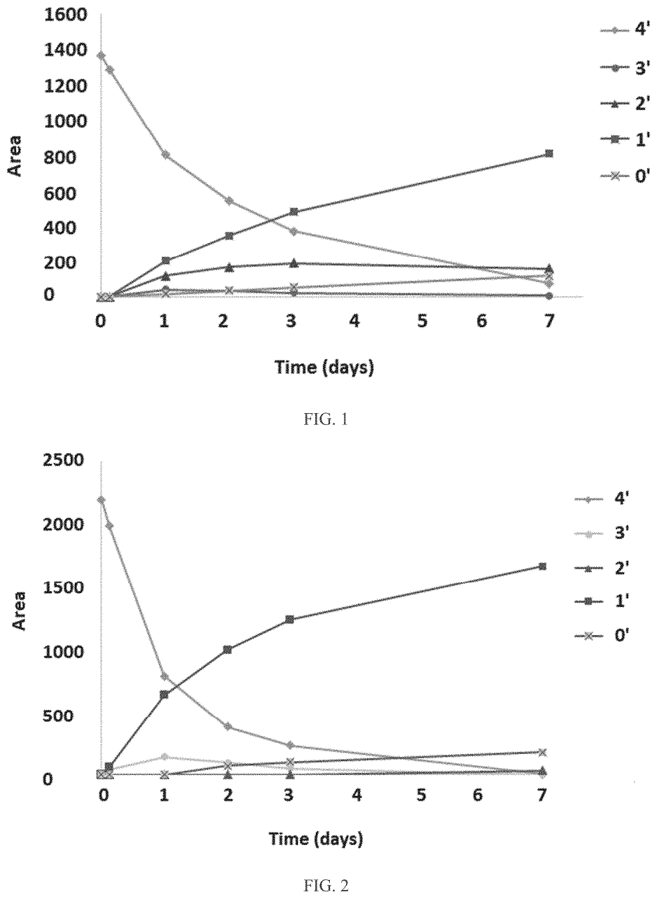

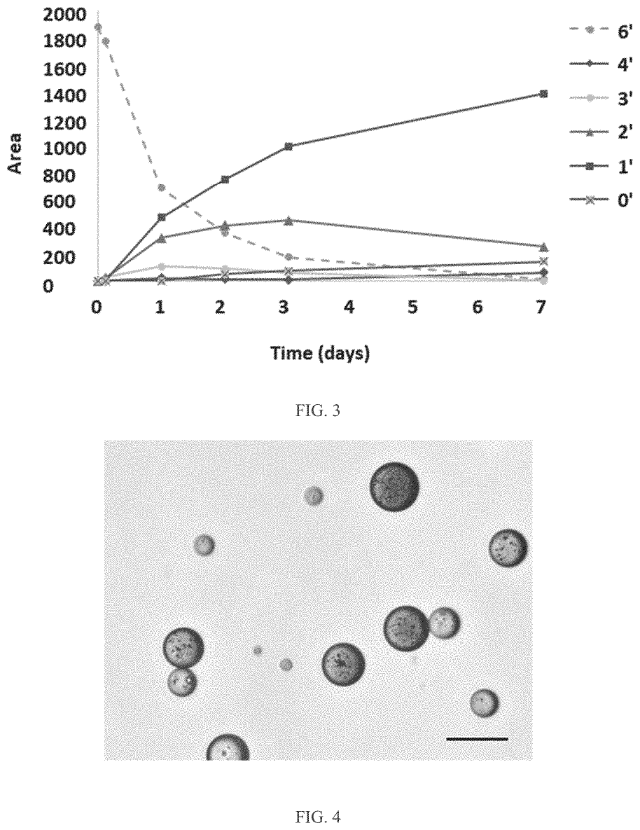

Evaluation of the degradation kinetics of the prodrugs of bumetanide and furosemide is illustrated in FIG. 1, FIG. 2, and FIG. 3. Complete degradation of the prodrugs was achieved by approximately day 7. Over that period, the predominant degradant generated was the prodrug conjugate with one lactide unit conjugated to the parent compound. By day 7, approximately 9% of the prodrug was converted to the free parent compound.

### Example 17. Encapsulation of Conjugates in Polymer Microparticles

### Materials

poly(D,L-lactic-co-glycolic acid (PLGA, 85:15 lactic acid to glycolic acid ratio, 5A, Evonik)
poly(D,L-lactic-co-glycolic acid (PLGA, 50:50 lactic acid to glycolic acid ratio)-poly(ethylene glycol)5000
poly(D,L-lactide, 4.5 A, Evonik)
poly vinyl alcohol (Mr ˜25K, 88% hydrolyzed, Polysciences)
Phosphate-buffered saline (pH 7.4)
Ultrapure cell culture grade water
All other chemicals were A.C.S. reagent grade (VWR)

### Microparticle Preparation

Microencapsulation of prodrugs of bumetanide and furosemide was achieved using an oil-in-water (o/w) emulsion/solvent evaporation method. The polymer was initially dissolved in a water immiscible organic solvent to which dissolved drug was added. Briefly, the polymers PLGA (LA:GA=85:15, 5A) or PLA (140-200 mg/mL) and PLGA_{50/50}-PEG₅ₖ (1.4-2 mg/mL) was dissolved in 2 mL of methylene chloride. The prodrug (15% theoretical loading) was dissolved in 1 mL of DMSO after vigorous vortexing and ultrasonication in a bath sonicator and added to the polymer solution. The aqueous phase consisted of 200 mL of PBS or water with 1% PVA as a surfactant to stabilize the emulsification. The dispersed phase was rapidly added to the aqueous phase and allowed to mix at 3400 rpms for 1 minute to generate an oil-in-water emulsion and disperse the materials as droplets. The volatile organic solution was allowed to evaporate under constant stirring at 500 rpms for 2 hours at room temperature. The particle suspension was allowed to settle for 30 minutes, after which the solution was decanted and remaining particles were collected, suspended in distilled deionized water, and centrifuged at 1000 rpms for 5 minutes. This process was repeated 3 times to remove any residual solvent. The pellet was collected and lyophilized overnight.

### Particle Size

Particle size and size distribution was determined using a Beckman Coulter Multsizer IV with a 100 μm diameter aperture based on a sample size of at least 50,000 counts. Particle size is expressed as volume-weighted mean diameters. Briefly, 2-5 mg of particles were suspended in 1 mL of double distilled water and added to a beaker containing 100 mL of ISOTON II solution. Measurements were obtained once the coincidence of particles reached 6-10%.

### Drug Loading

To determine the % drug loading (DL), 10 mg of particles was weighed into a glass scintillation vial and dissolved with 10 mL of MeCN:water (1:1, v/v). The solution was filtered through a 0.2 μm nylon syringe filter and the drug content was determined by RP-HPLC referenced against a standard calibration curve.

Microparticles ranged in size between 23-28 μm in volume weighted mean diameter. Of significance, the prodrugs of bumetanide and furosemide was significantly more amenable to microencapsulation in comparison to the free drugs. Percent drug loading of bumetanide and furosemide prodrug was approximately 3 and 9-fold higher than free drug, respectively. In addition, encapsulation efficiency was significantly higher than free drug. Encapsulation efficiency was greater than 95% for prodrugs of bumetanide and furosemide.

TABLE 3
Formulation parameters and physicochemical properties of microparticles
encapsulating free drug and prodrugs of bumetanide and furosemide

| | | | | | Mean | | |
|---|---|---|---|---|---|---|---|
| | | | Polymer | % | Particle | | |
| Compd | Compd | Backbone | Conc | Theoretical | Size | | % |
| Name | # | Polymer | (mg/mL) | Loading | (μm) | SD | DL |
| Bumetanide | — | PLGA7525 4A + | 260 | 15.0 | 23.66 | 8.28 | 5.03 |
| | | 1% | | | | | |
| | | PEG-PLGA5050 | | | | | |
| | | (99% PLGA + | | | | | |
| | | 1% PEG-PLGA) | | | | | |
| Furosemide | — | PLGA7525 4A + | 260 | 15.0 | 22.78 | 7.70 | 1.68 |
| | | 1% | | | | | |
| | | PEG-PLGA5050 | | | | | |
| | | (99% PLGA + | | | | | |
| | | 1% PEG-PLGA) | | | | | |
| Bumetanide- | 2 | 77/22 (PLA | 260 | 15.0 | 26.98 | 8.53 | 14.38 |
| Ethyl | | 4.5A/PLGA8515 | | | | | |
| PLA(n = 4) | | 5A) + 1% | | | | | |
| | | PEG-PLGA5050 | | | | | |
| | | (99% PLA, | | | | | |
| | | PLGA blend + | | | | | |
| | | 1% PEG-PLGA) | | | | | |
| Furosemide- | 5 | 77/22 (PLA | 260 | 15.0 | 27.74 | 9.01 | 14.60 |
| Ethyl | | 4.5A/PLGA8515 | | | | | |
| PLA(n = 6) | | 5A) + 1% | | | | | |
| | | PEG-PLGA5050 | | | | | |
| | | (99% PLA, | | | | | |
| | | PLGA blend + | | | | | |
| | | 1% PEG-PLGA) | | | | | |

### Particle Morphology

Particle morphology was assessed using a Nikon Eclipse TS-100 light microscope. Briefly, 3-5 mg of furosemide-ethyl PLA(n=6) (Compound 5) particles were suspended in 1 mL of water. A volume of 10 uL of the particle suspension was transferred onto a glass slide and imaged directly (FIG. 4).

### Drug Release

In vitro drug release kinetics was evaluated in a release medium of PBS and 1% Tween 20 (pH 7.4). Briefly, 10 mg of particles were transferred to glass scintillation vials and 4 mL of the release medium was added to suspend the particles. Samples were prepared in duplicate. The particles were mixed by gentle vortexing and incubated on an orbital shaker at 150 rpm at 37° C. At various time points, 3 mL of release media was collected and analyzed for drug content and 3 mL of fresh media was added to replace the sample that was collected. Collected release samples were frozen and stored at −80° C. until analysis for drug content. The collected samples were filtered through a 0.2 μm syringe filter and analyzed by RP-HPLC.

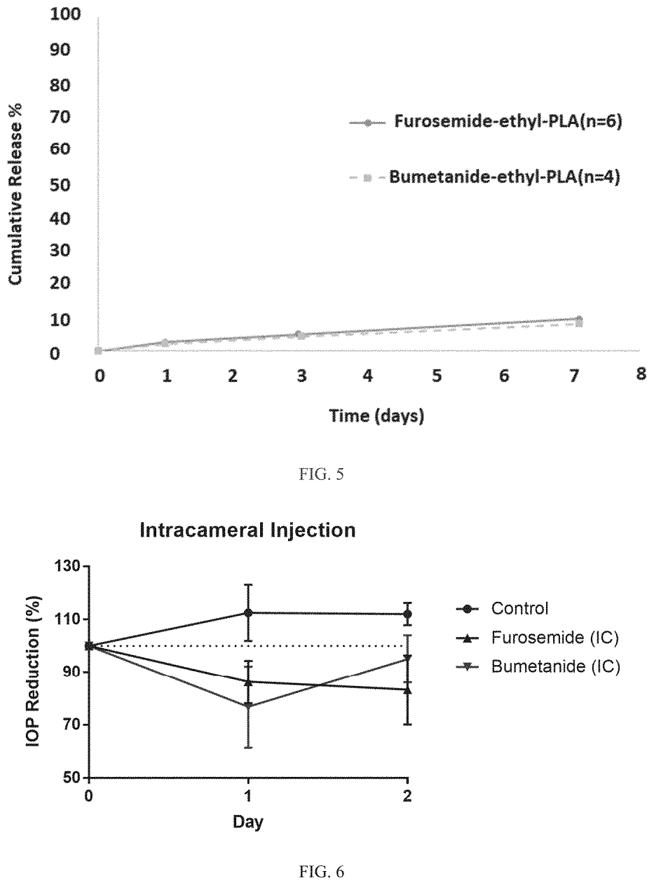

Release kinetics for furosemide-ethyl PLA(n=6) (Compound 5) and bumetanide-ethyl PLA(n=4) (Compound 2) from microparticles is illustrated in FIG. 5. Both particle formulations exhibited a low burst release of approximately 2-3% within the first day. Release was linear from day 1-7, with less than 10% cumulative drug released by day 7.

### Example 18. Furosemide and Bumetanide Lower IOP in African Green Monkeys

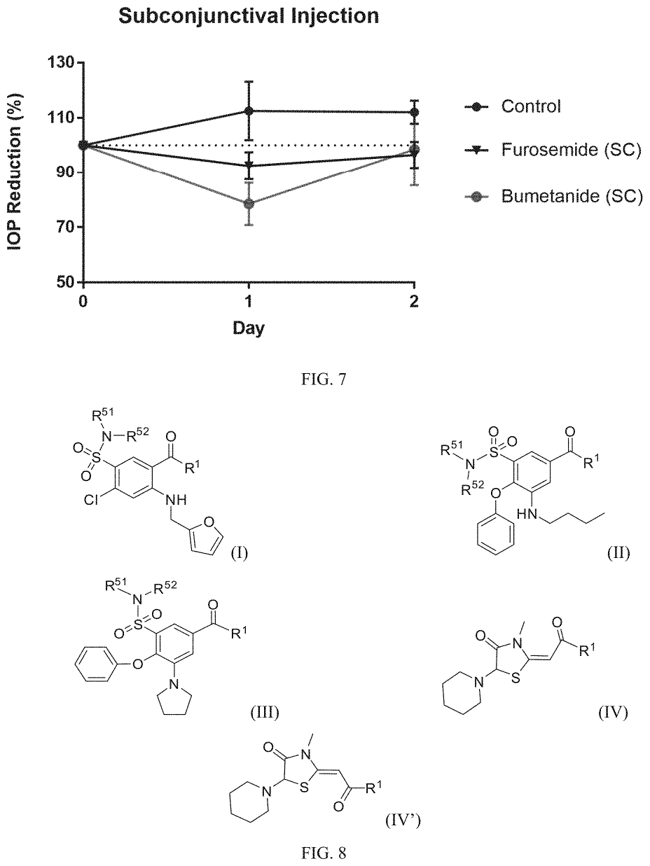

Two loop diuretic compounds, furosemide and bumetanide, were evaluated in African green monkeys for their potential to treat ocular hypertension and glaucoma. Commercially available bumetanide injection solution (0.25 mg/mL) was used directly in the study. Furosemide injection solution (8 mg/mL) was first diluted in a phosphate buffered saline solution to 0.25 mg/mL prior to use in the study. The diluted bumetanide and furosemide solutions were administrated in African green monkeys via intracameral (IC, 10 μL) (FIG. 6) or subconjunctival (SC, 20 μL) (FIG. 7) route and the effect of the compounds was evaluated by measuring the IOP on Day 0, 1 and 2 using a TonoVet (iCare, Finland) tonometer. As shown in FIG. 6 and FIG. 7, a significant IOP reduction (up to ˜20%) was observed in animals treated with bumetanide or furosemide. No ocular toxicity findings were observed by ophthalmic examination.

### Example 19. Additional Non-Limiting Examples of the Present Invention

Table 4 and Table 5 illustrate non-limiting examples of compounds of the present invention.

TABLE 4
Compounds of the Present Invention

| Compd No. | |
|---|---|
| 1 | 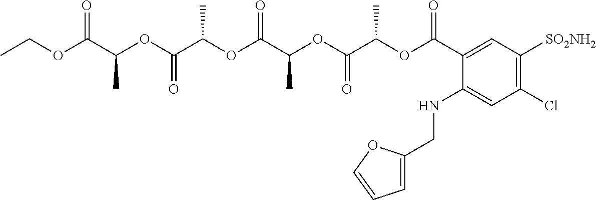 [SMILES: CCOC(=O)[C@H](C)OC(=O)[C@H](C)OC(=O)[C@H](C)OC(=O)[C@H](C)OC(=O)c1cc(S(N)(=O)=O)c(Cl)cc1NCc1ccco1] |
| 2 | 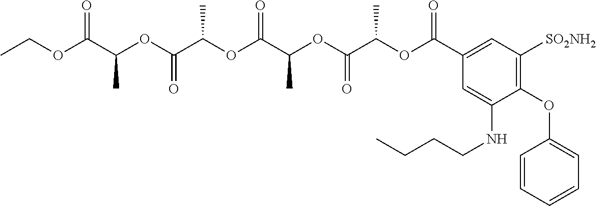 [SMILES: CCCCNc1cc(C(=O)O[C@@H](C)C(=O)O[C@@H](C)C(=O)O[C@@H](C)C(=O)O[C@@H](C)C(=O)OCC)cc(S(N)(=O)=O)c1Oc1ccccc1] |
| 3 | 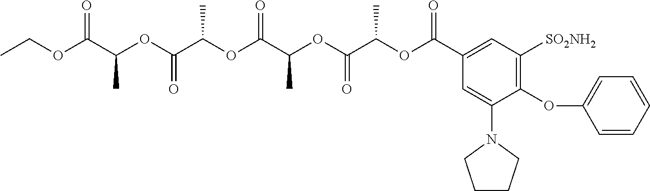 [SMILES: CCOC(=O)[C@H](C)OC(=O)[C@H](C)OC(=O)[C@H](C)OC(=O)[C@H](C)OC(=O)c1cc(N2CCCC2)c(Oc2ccccc2)c(S(N)(=O)=O)c1] |
| 4 | 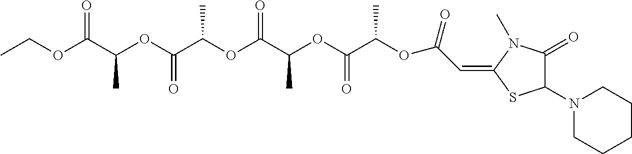 [SMILES: CCOC(=O)[C@H](C)OC(=O)[C@H](C)OC(=O)[C@H](C)OC(=O)[C@H](C)OC(=O)/C=C1/SC(N2CCCCC2)C(=O)N1C] |
| 5 | 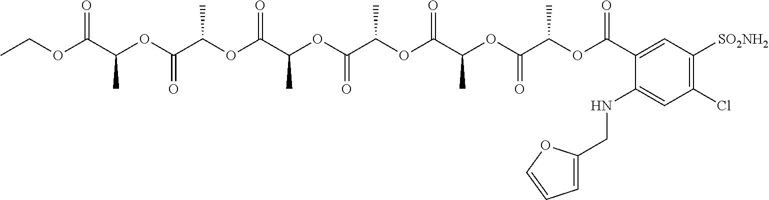 [SMILES: CCOC(=O)[C@H](C)OC(=O)[C@H](C)OC(=O)[C@H](C)OC(=O)[C@H](C)OC(=O)[C@H](C)OC(=O)[C@H](C)OC(=O)c1cc(S(N)(=O)=O)c(Cl)cc1NCc1ccco1] |
| 6 | 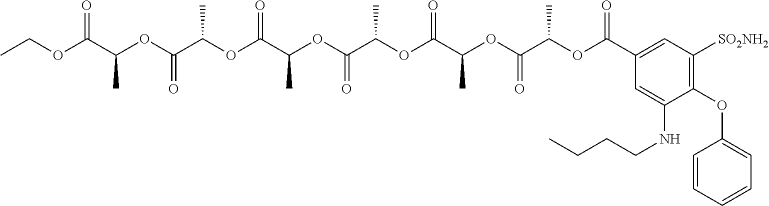 [SMILES: CCCCNc1cc(C(=O)O[C@@H](C)C(=O)O[C@@H](C)C(=O)O[C@@H](C)C(=O)O[C@@H](C)C(=O)O[C@@H](C)C(=O)O[C@@H](C)C(=O)OCC)cc(S(N)(=O)=O)c1Oc1ccccc1] |
| 7 | 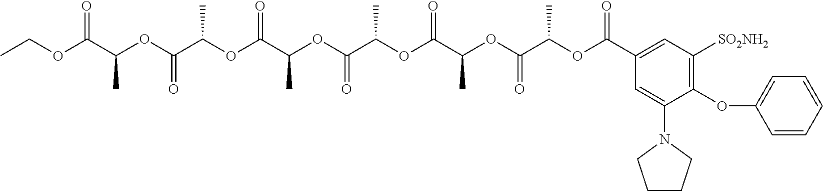 [SMILES: CCOC(=O)[C@H](C)OC(=O)[C@H](C)OC(=O)[C@H](C)OC(=O)[C@H](C)OC(=O)[C@H](C)OC(=O)[C@H](C)OC(=O)c1cc(N2CCCC2)c(Oc2ccccc2)c(S(N)(=O)=O)c1] |
| 8 | 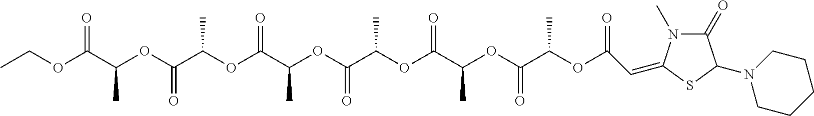 [SMILES: CCOC(=O)[C@H](C)OC(=O)[C@H](C)OC(=O)[C@H](C)OC(=O)[C@H](C)OC(=O)[C@H](C)OC(=O)[C@H](C)OC(=O)/C=C1/SC(N2CCCCC2)C(=O)N1C] |

TABLE 5
Compounds of the Present Invention

| Compd No. | |
|---|---|
| 9 | 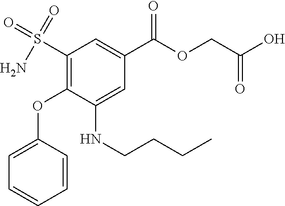 [SMILES: CCCCNc1cc(C(=O)OCC(=O)O)cc(S(N)(=O)=O)c1Oc1ccccc1] |
| 10 | 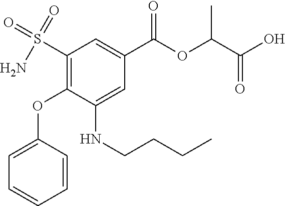 [SMILES: CCCCNc1cc(C(=O)OC(C)C(=O)O)cc(S(N)(=O)=O)c1Oc1ccccc1] |
| 11 | 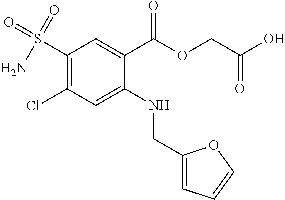 [SMILES: NS(=O)(=O)c1cc(C(=O)OCC(=O)O)c(NCc2ccco2)cc1Cl] |
| 12 | 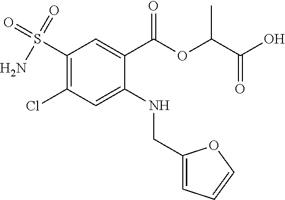 [SMILES: CC(OC(=O)c1cc(S(N)(=O)=O)c(Cl)cc1NCc1ccco1)C(=O)O] |
| 13 | 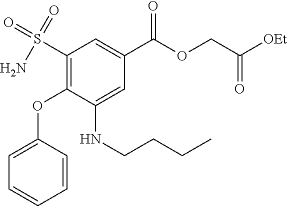 [SMILES: CCCCNc1cc(C(=O)OCC(=O)OCC)cc(S(N)(=O)=O)c1Oc1ccccc1] |
| 14 | 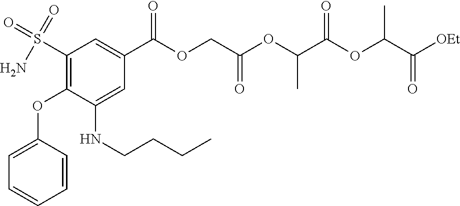 [SMILES: CCCCNc1cc(C(=O)OCC(=O)OC(C)C(=O)OC(C)C(=O)OCC)cc(S(N)(=O)=O)c1Oc1ccccc1] |
| 15 | 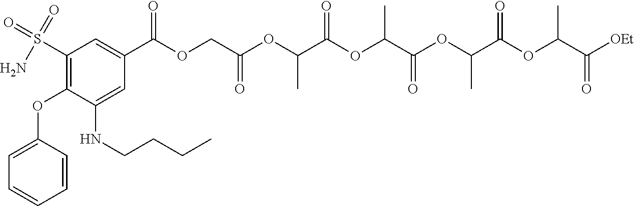 [SMILES: CCCCNc1cc(C(=O)OCC(=O)OC(C)C(=O)OC(C)C(=O)OC(C)C(=O)OC(C)C(=O)OCC)cc(S(N)(=O)=O)c1Oc1ccccc1] |
| 16 | 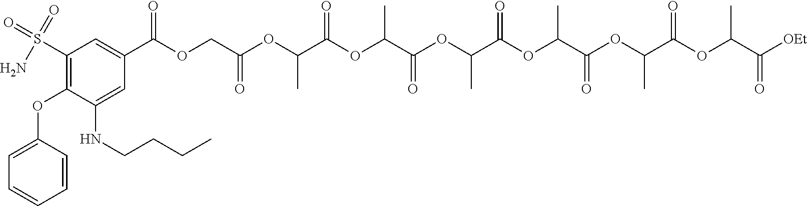 [SMILES: CCCCNc1cc(C(=O)OCC(=O)OC(C)C(=O)OC(C)C(=O)OC(C)C(=O)OC(C)C(=O)OC(C)C(=O)OC(C)C(=O)OCC)cc(S(N)(=O)=O)c1Oc1ccccc1] |
| 17 | 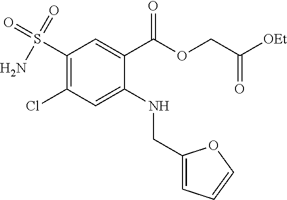 [SMILES: CCOC(=O)COC(=O)c1cc(S(N)(=O)=O)c(Cl)cc1NCc1ccco1] |
| 18 | 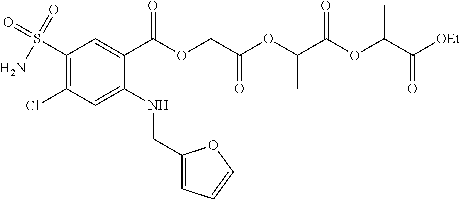 [SMILES: CCOC(=O)C(C)OC(=O)C(C)OC(=O)COC(=O)c1cc(S(N)(=O)=O)c(Cl)cc1NCc1ccco1] |
| 19 | 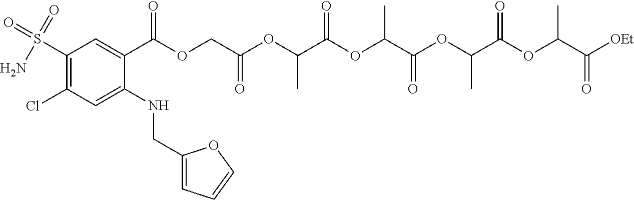 [SMILES: CCOC(=O)C(C)OC(=O)C(C)OC(=O)C(C)OC(=O)C(C)OC(=O)COC(=O)c1cc(S(N)(=O)=O)c(Cl)cc1NCc1ccco1] |
| 20 | 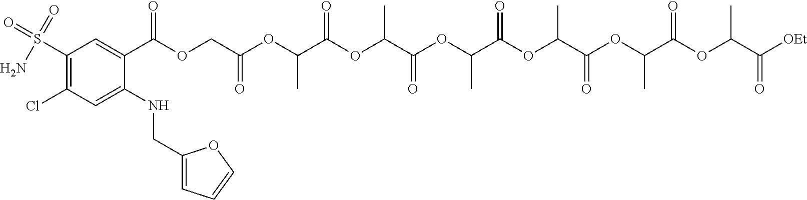 [SMILES: CCOC(=O)C(C)OC(=O)C(C)OC(=O)C(C)OC(=O)C(C)OC(=O)C(C)OC(=O)C(C)OC(=O)COC(=O)c1cc(S(N)(=O)=O)c(Cl)cc1NCc1ccco1] |
| 21 | 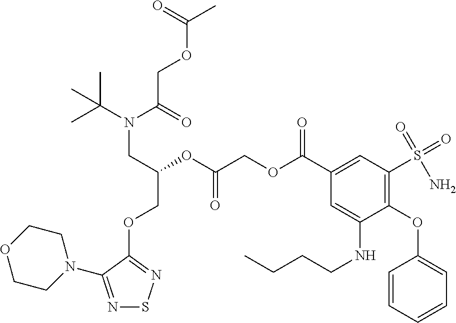 [SMILES: CCCCNc1cc(C(=O)OCC(=O)O[C@H](COc2nsnc2N2CCOCC2)CN(C(=O)COC(C)=O)C(C)(C)C)cc(S(N)(=O)=O)c1Oc1ccccc1] |
| 22 | 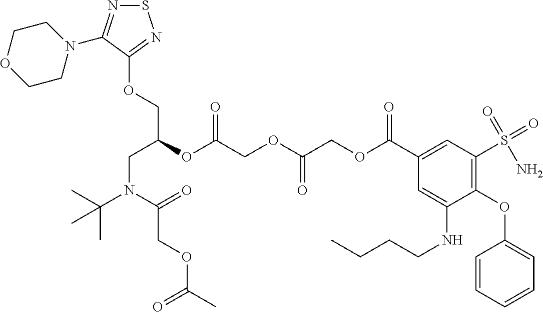 [SMILES: CCCCNc1cc(C(=O)OCC(=O)OCC(=O)O[C@H](COc2nsnc2N2CCOCC2)CN(C(=O)COC(C)=O)C(C)(C)C)cc(S(N)(=O)=O)c1Oc1ccccc1] |
| 23 | 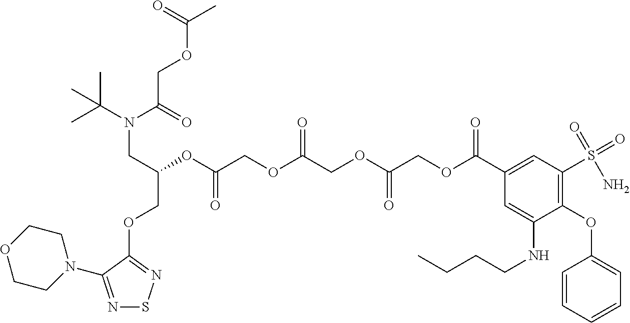 [SMILES: CCCCNc1cc(C(=O)OCC(=O)OCC(=O)OCC(=O)O[C@H](COc2nsnc2N2CCOCC2)CN(C(=O)COC(C)=O)C(C)(C)C)cc(S(N)(=O)=O)c1Oc1ccccc1] |
| 24 | 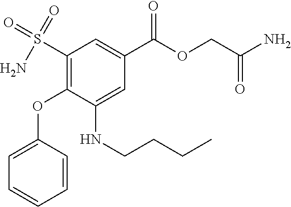 [SMILES: CCCCNc1cc(C(=O)OCC(N)=O)cc(S(N)(=O)=O)c1Oc1ccccc1] |
| 25 | 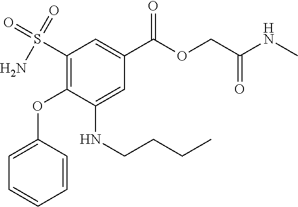 [SMILES: CCCCNc1cc(C(=O)OCC(=O)NC)cc(S(N)(=O)=O)c1Oc1ccccc1] |
| 26 | 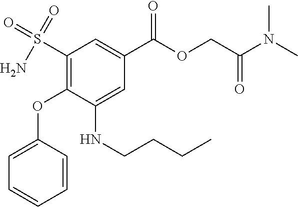 [SMILES: CCCCNc1cc(C(=O)OCC(=O)N(C)C)cc(S(N)(=O)=O)c1Oc1ccccc1] |
| 27 | 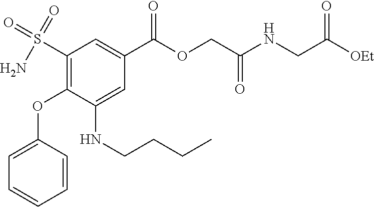 [SMILES: CCCCNc1cc(C(=O)OCC(=O)NCC(=O)OCC)cc(S(N)(=O)=O)c1Oc1ccccc1] |
| 28 | 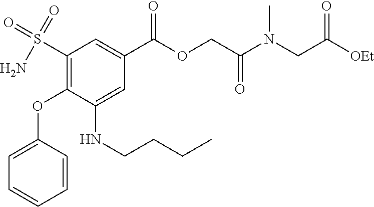 [SMILES: CCCCNc1cc(C(=O)OCC(=O)N(C)CC(=O)OCC)cc(S(N)(=O)=O)c1Oc1ccccc1] |
| 29 | 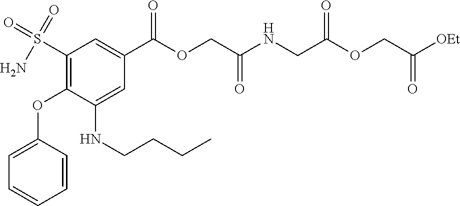 [SMILES: CCCCNc1cc(C(=O)OCC(=O)NCC(=O)OCC(=O)OCC)cc(S(N)(=O)=O)c1Oc1ccccc1] |
| 30 | 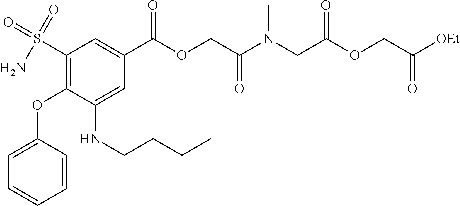 [SMILES: CCCCNc1cc(C(=O)OCC(=O)N(C)CC(=O)OCC(=O)OCC)cc(S(N)(=O)=O)c1Oc1ccccc1] |
| 31 | 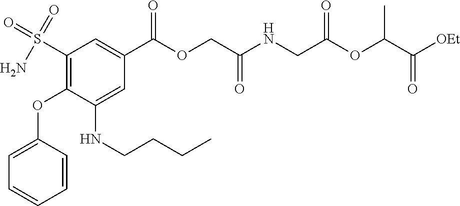 [SMILES: CCCCNc1cc(C(=O)OCC(=O)NCC(=O)OC(C)C(=O)OCC)cc(S(N)(=O)=O)c1Oc1ccccc1] |
| 32 | 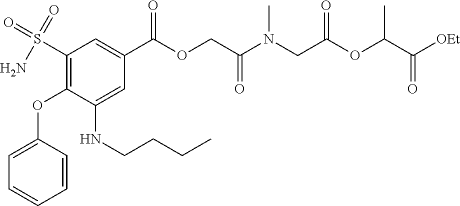 [SMILES: CCCCNc1cc(C(=O)OCC(=O)N(C)CC(=O)OC(C)C(=O)OCC)cc(S(N)(=O)=O)c1Oc1ccccc1] |
| 33 | 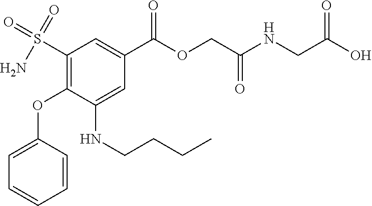 [SMILES: CCCCNc1cc(C(=O)OCC(=O)NCC(=O)O)cc(S(N)(=O)=O)c1Oc1ccccc1] |
| 34 | 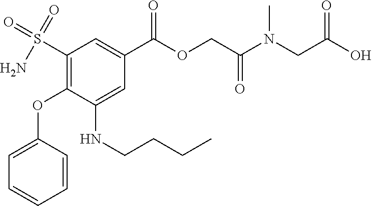 [SMILES: CCCCNc1cc(C(=O)OCC(=O)N(C)CC(=O)O)cc(S(N)(=O)=O)c1Oc1ccccc1] |
| 35 | 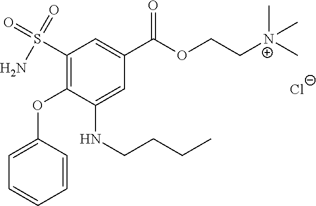 [SMILES: CCCCNc1cc(C(=O)OCC[N+](C)(C)C)cc(S(N)(=O)=O)c1Oc1ccccc1 ; [Cl-]] |
| 36 | 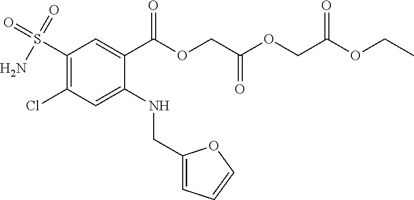 [SMILES: CCOC(=O)COC(=O)COC(=O)c1cc(S(N)(=O)=O)c(Cl)cc1NCc1ccco1] |
| 37 | 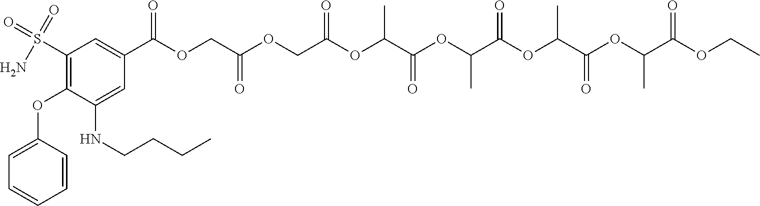 [SMILES: CCCCNc1cc(C(=O)OCC(=O)OCC(=O)OC(C)C(=O)OC(C)C(=O)OC(C)C(=O)OC(C)C(=O)OCC)cc(S(N)(=O)=O)c1Oc1ccccc1] |
| 38 | 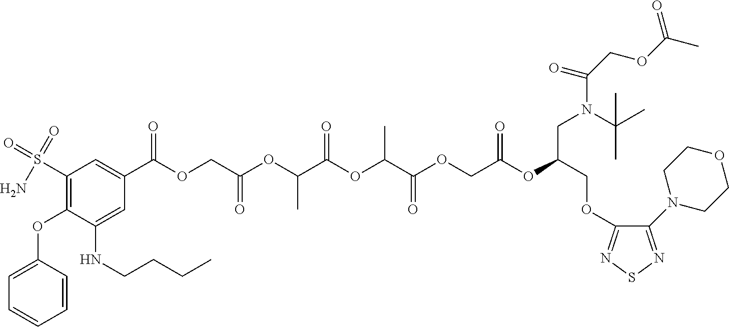 [SMILES: CCCCNc1cc(C(=O)OCC(=O)OC(C)C(=O)OC(C)C(=O)OCC(=O)O[C@H](COc2nsnc2N2CCOCC2)CN(C(=O)COC(C)=O)C(C)(C)C)cc(S(N)(=O)=O)c1Oc1ccccc1] |
| 39 | 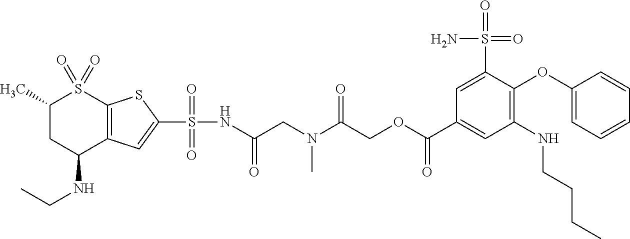 [SMILES: CCCCNc1cc(C(=O)OCC(=O)N(C)CC(=O)NS(=O)(=O)c2cc3c(s2)S(=O)(=O)[C@@H](C)C[C@@H]3NCC)cc(S(N)(=O)=O)c1Oc1ccccc1] |
| 40 | 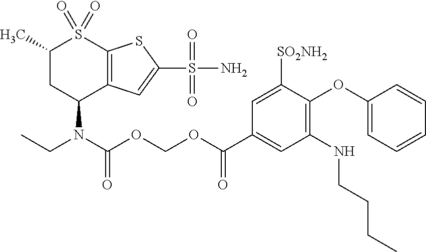 [SMILES: CCCCNc1cc(C(=O)OCOC(=O)N(CC)[C@H]2C[C@H](C)S(=O)(=O)c3sc(S(N)(=O)=O)cc32)cc(S(N)(=O)=O)c1Oc1ccccc1] |
| 41 | 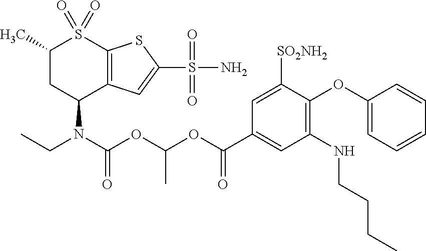 [SMILES: CCCCNc1cc(C(=O)OC(C)OC(=O)N(CC)[C@H]2C[C@H](C)S(=O)(=O)c3sc(S(N)(=O)=O)cc32)cc(S(N)(=O)=O)c1Oc1ccccc1] |
| 42 | 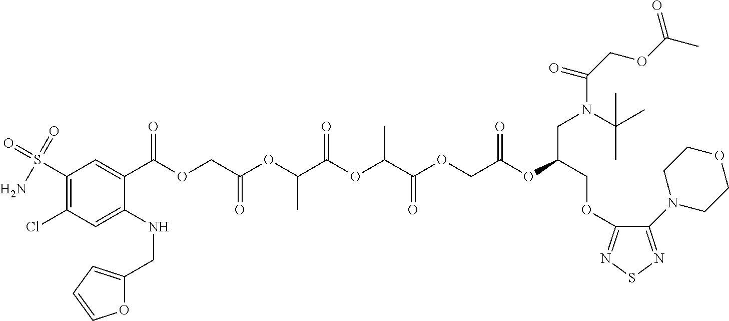 [SMILES: CC(=O)OCC(=O)N(C[C@@H](COc1nsnc1N1CCOCC1)OC(=O)COC(=O)C(C)OC(=O)C(C)OC(=O)COC(=O)c1cc(S(N)(=O)=O)c(Cl)cc1NCc1ccco1)C(C)(C)C] |
| 43 | 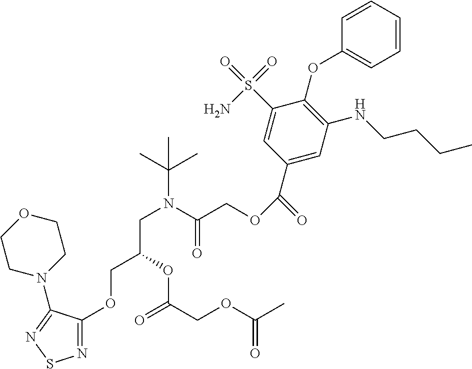 [SMILES: CCCCNc1cc(C(=O)OCC(=O)N(C[C@@H](COc2nsnc2N2CCOCC2)OC(=O)COC(C)=O)C(C)(C)C)cc(S(N)(=O)=O)c1Oc1ccccc1] |
| 44 | 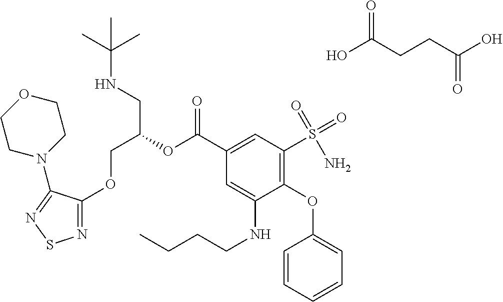 [SMILES: CCCCNc1cc(C(=O)O[C@@H](CNC(C)(C)C)COc2nsnc2N2CCOCC2)cc(S(N)(=O)=O)c1Oc1ccccc1 ; O=C(O)CCC(=O)O] |
| 45 | 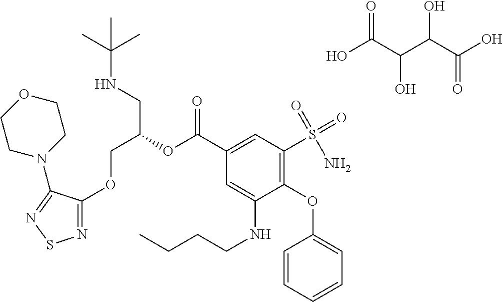 [SMILES: CCCCNc1cc(C(=O)O[C@@H](CNC(C)(C)C)COc2nsnc2N2CCOCC2)cc(S(N)(=O)=O)c1Oc1ccccc1 ; O=C(O)C(O)C(O)C(=O)O] |
| 46 | 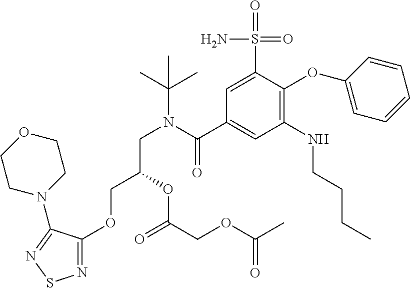 [SMILES: CCCCNc1cc(C(=O)N(C[C@@H](COc2nsnc2N2CCOCC2)OC(=O)COC(C)=O)C(C)(C)C)cc(S(N)(=O)=O)c1Oc1ccccc1] |
| 47 | 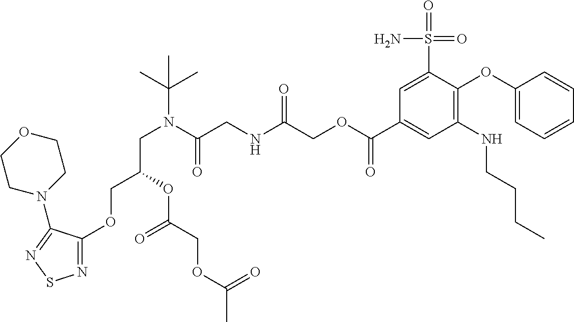 [SMILES: CCCCNc1cc(C(=O)OCC(=O)NCC(=O)N(C[C@@H](COc2nsnc2N2CCOCC2)OC(=O)COC(C)=O)C(C)(C)C)cc(S(N)(=O)=O)c1Oc1ccccc1] |
| 48 | 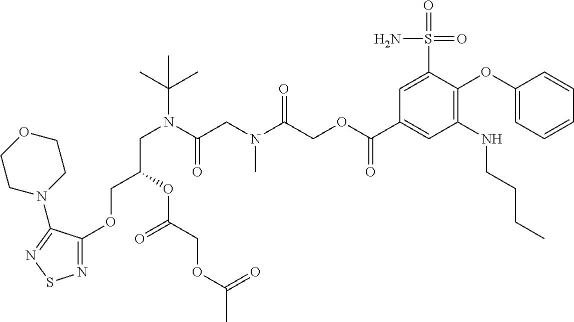 [SMILES: CCCCNc1cc(C(=O)OCC(=O)N(C)CC(=O)N(C[C@@H](COc2nsnc2N2CCOCC2)OC(=O)COC(C)=O)C(C)(C)C)cc(S(N)(=O)=O)c1Oc1ccccc1] |
| 49 | 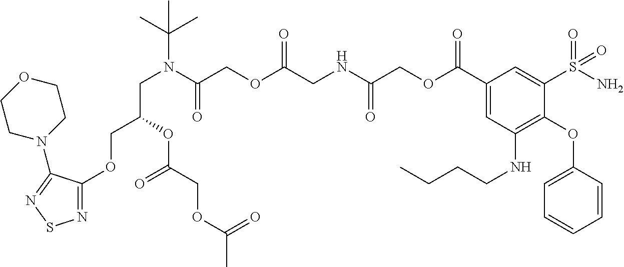 [SMILES: CCCCNc1cc(C(=O)OCC(=O)NCC(=O)OCC(=O)N(C[C@@H](COc2nsnc2N2CCOCC2)OC(=O)COC(C)=O)C(C)(C)C)cc(S(N)(=O)=O)c1Oc1ccccc1] |
| 50 | 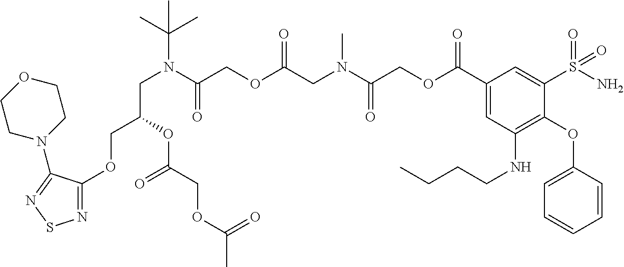 [SMILES: CCCCNc1cc(C(=O)OCC(=O)N(C)CC(=O)OCC(=O)N(C[C@@H](COc2nsnc2N2CCOCC2)OC(=O)COC(C)=O)C(C)(C)C)cc(S(N)(=O)=O)c1Oc1ccccc1] |
| 51 | 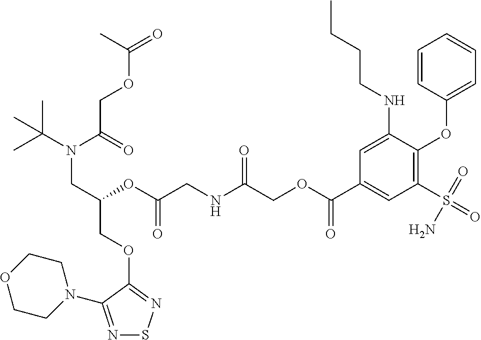 [SMILES: CCCCNc1cc(C(=O)OCC(=O)NCC(=O)O[C@H](COc2nsnc2N2CCOCC2)CN(C(=O)COC(C)=O)C(C)(C)C)cc(S(N)(=O)=O)c1Oc1ccccc1] |
| 52 | 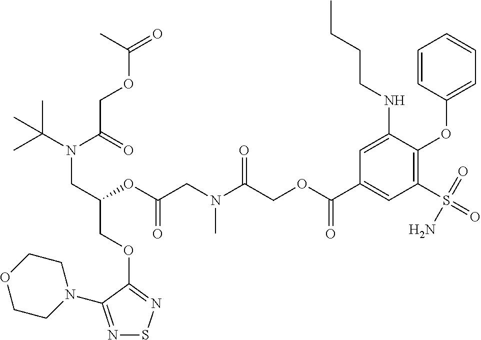 [SMILES: CCCCNc1cc(C(=O)OCC(=O)N(C)CC(=O)O[C@H](COc2nsnc2N2CCOCC2)CN(C(=O)COC(C)=O)C(C)(C)C)cc(S(N)(=O)=O)c1Oc1ccccc1] |
| 53 | 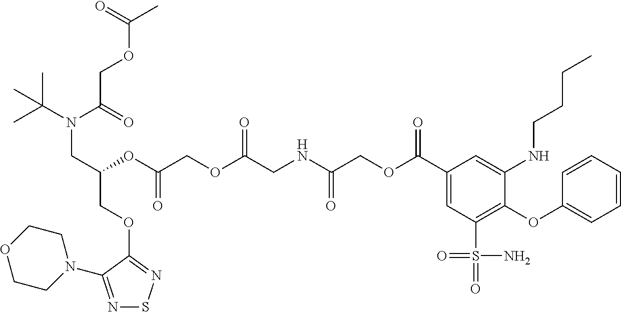 [SMILES: CCCCNc1cc(C(=O)OCC(=O)NCC(=O)OCC(=O)O[C@H](COc2nsnc2N2CCOCC2)CN(C(=O)COC(C)=O)C(C)(C)C)cc(S(N)(=O)=O)c1Oc1ccccc1] |
| 54 | 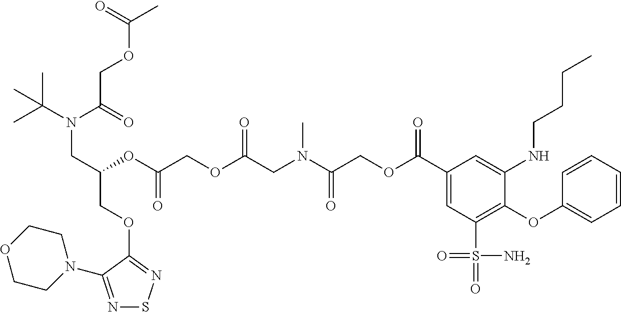 [SMILES: CCCCNc1cc(C(=O)OCC(=O)N(C)CC(=O)OCC(=O)O[C@H](COc2nsnc2N2CCOCC2)CN(C(=O)COC(C)=O)C(C)(C)C)cc(S(N)(=O)=O)c1Oc1ccccc1] |
| 55 | 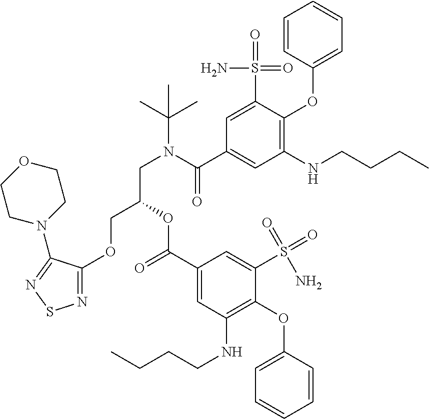 [SMILES: CCCCNc1cc(C(=O)O[C@H](COc2nsnc2N2CCOCC2)CN(C(=O)c2cc(NCCCC)c(Oc3ccccc3)c(S(N)(=O)=O)c2)C(C)(C)C)cc(S(N)(=O)=O)c1Oc1ccccc1] |
| 56 | 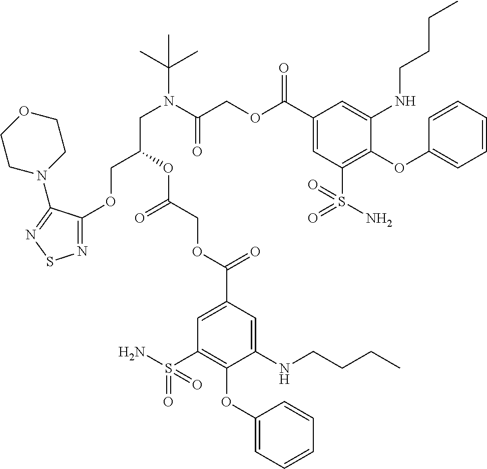 [SMILES: CCCCNc1cc(C(=O)OCC(=O)O[C@H](COc2nsnc2N2CCOCC2)CN(C(=O)COC(=O)c2cc(NCCCC)c(Oc3ccccc3)c(S(N)(=O)=O)c2)C(C)(C)C)cc(S(N)(=O)=O)c1Oc1ccccc1] |
| 57 | 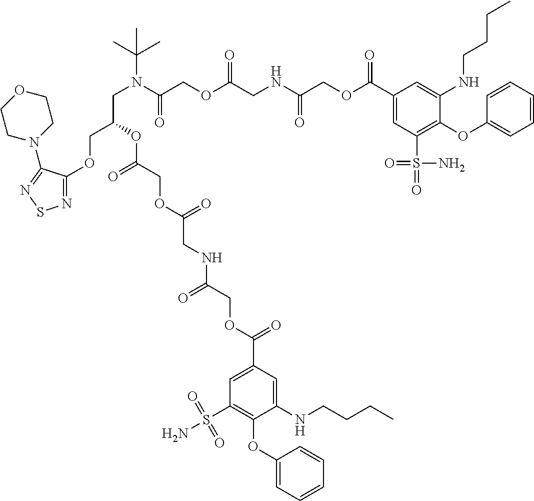 [SMILES: CCCCNc1cc(C(=O)OCC(=O)NCC(=O)OCC(=O)O[C@H](COc2nsnc2N2CCOCC2)CN(C(=O)COC(=O)CNC(=O)COC(=O)c2cc(NCCCC)c(Oc3ccccc3)c(S(N)(=O)=O)c2)C(C)(C)C)cc(S(N)(=O)=O)c1Oc1ccccc1] |
| 58 | 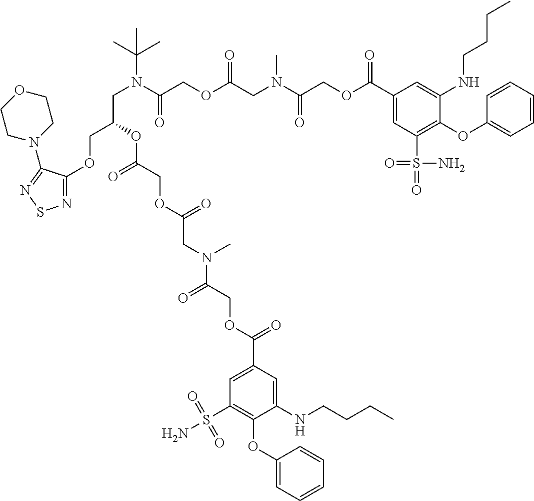 [SMILES: CCCCNc1cc(C(=O)OCC(=O)N(C)CC(=O)OCC(=O)O[C@H](COc2nsnc2N2CCOCC2)CN(C(=O)COC(=O)CN(C)C(=O)COC(=O)c2cc(NCCCC)c(Oc3ccccc3)c(S(N)(=O)=O)c2)C(C)(C)C)cc(S(N)(=O)=O)c1Oc1ccccc1] |
| 59 | 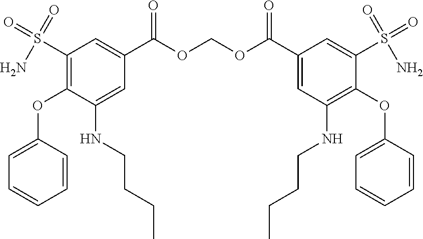 [SMILES: CCCCNc1cc(C(=O)OCOC(=O)c2cc(NCCCC)c(Oc3ccccc3)c(S(N)(=O)=O)c2)cc(S(N)(=O)=O)c1Oc1ccccc1] |
| 60 | 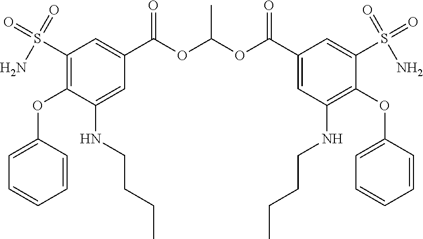 [SMILES: CCCCNc1cc(C(=O)OC(C)OC(=O)c2cc(NCCCC)c(Oc3ccccc3)c(S(N)(=O)=O)c2)cc(S(N)(=O)=O)c1Oc1ccccc1] |
| 61 | 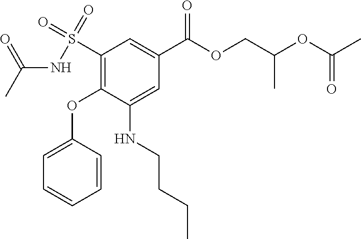 [SMILES: CCCCNc1cc(C(=O)OCC(C)OC(C)=O)cc(S(=O)(=O)NC(C)=O)c1Oc1ccccc1] |
| 62 | 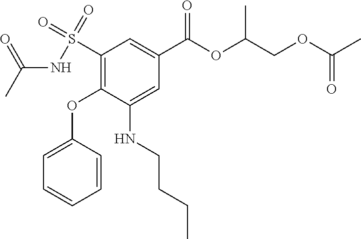 [SMILES: CCCCNc1cc(C(=O)OC(C)COC(C)=O)cc(S(=O)(=O)NC(C)=O)c1Oc1ccccc1] |
| 63 | 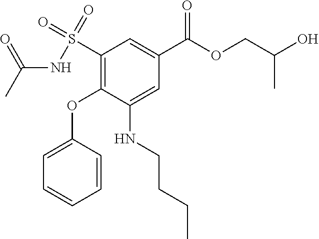 [SMILES: CCCCNc1cc(C(=O)OCC(C)O)cc(S(=O)(=O)NC(C)=O)c1Oc1ccccc1] |
| 64 | 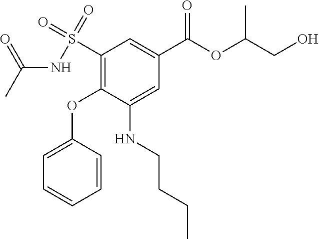 [SMILES: CCCCNc1cc(C(=O)OC(C)CO)cc(S(=O)(=O)NC(C)=O)c1Oc1ccccc1] |
| 65 | 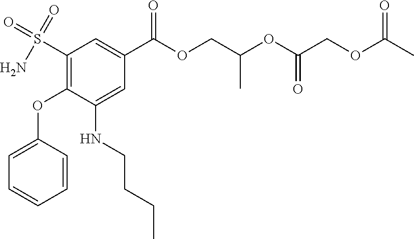 [SMILES: CCCCNc1cc(C(=O)OCC(C)OC(=O)COC(C)=O)cc(S(N)(=O)=O)c1Oc1ccccc1] |
| 66 | 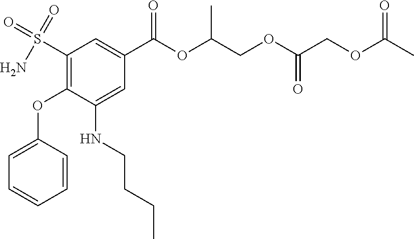 [SMILES: CCCCNc1cc(C(=O)OC(C)COC(=O)COC(C)=O)cc(S(N)(=O)=O)c1Oc1ccccc1] |
| 67 | 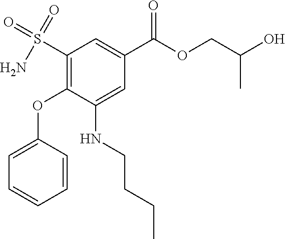 [SMILES: CCCCNc1cc(C(=O)OCC(C)O)cc(S(N)(=O)=O)c1Oc1ccccc1] |
| 68 | 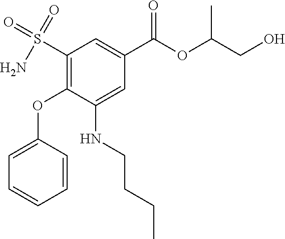 [SMILES: CCCCNc1cc(C(=O)OC(C)CO)cc(S(N)(=O)=O)c1Oc1ccccc1] |
| 69 | 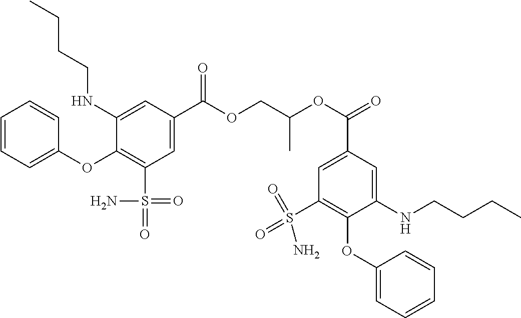 [SMILES: CCCCNc1cc(C(=O)OCC(C)OC(=O)c2cc(NCCCC)c(Oc3ccccc3)c(S(N)(=O)=O)c2)cc(S(N)(=O)=O)c1Oc1ccccc1] |
| 70 | 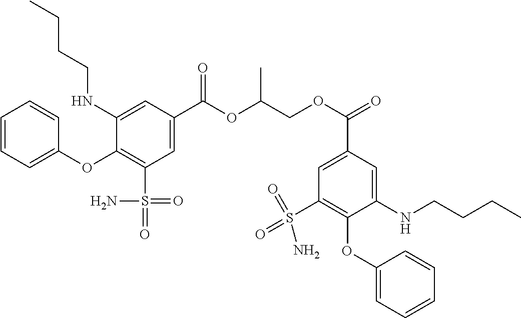 [SMILES: CCCCNc1cc(C(=O)OCC(C)OC(=O)c2cc(NCCCC)c(Oc3ccccc3)c(S(N)(=O)=O)c2)cc(S(N)(=O)=O)c1Oc1ccccc1] |
| 71 | 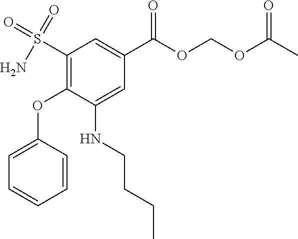 [SMILES: CCCCNc1cc(C(=O)OCOC(C)=O)cc(S(N)(=O)=O)c1Oc1ccccc1] |
| 72 | 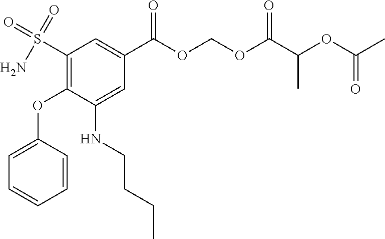 [SMILES: CCCCNc1cc(C(=O)OCOC(=O)C(C)OC(C)=O)cc(S(N)(=O)=O)c1Oc1ccccc1] |
| 73 | 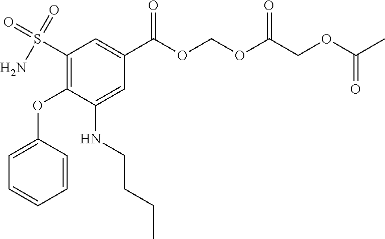 [SMILES: CCCCNc1cc(C(=O)OCOC(=O)COC(C)=O)cc(S(N)(=O)=O)c1Oc1ccccc1] |
| 74 | 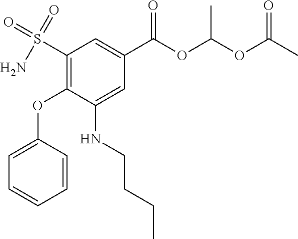 [SMILES: CCCCNc1cc(C(=O)OC(C)OC(C)=O)cc(S(N)(=O)=O)c1Oc1ccccc1] |
| 75 | 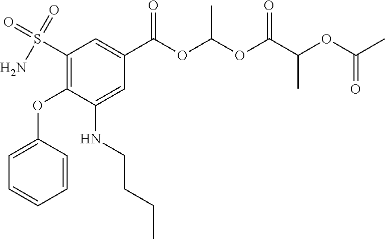 [SMILES: CCCCNc1cc(C(=O)OC(C)OC(=O)C(C)OC(C)=O)cc(S(N)(=O)=O)c1Oc1ccccc1] |
| 76 | 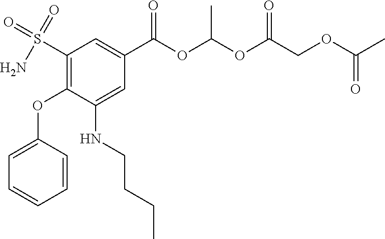 [SMILES: CCCCNc1cc(C(=O)OC(C)OC(=O)COC(C)=O)cc(S(N)(=O)=O)c1Oc1ccccc1] |
| 78 | 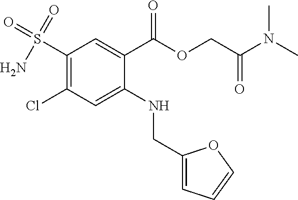 [SMILES: CN(C)C(=O)COC(=O)c1cc(S(N)(=O)=O)c(Cl)cc1NCc1ccco1] |
| 79 | 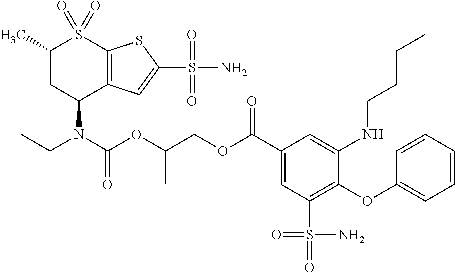 [SMILES: CCCCNc1cc(C(=O)OCC(C)OC(=O)N(CC)[C@H]2C[C@H](C)S(=O)(=O)c3sc(S(N)(=O)=O)cc32)cc(S(N)(=O)=O)c1Oc1ccccc1] |

This specification has been described with reference to embodiments of the invention. However, one of ordinary skill in the art appreciates that various modifications and changes can be made without departing from the scope of the invention as set forth herein. Accordingly, the specification is to be regarded in an illustrative rather than a restrictive sense, and all such modifications are intended to be included within the scope of invention.

What is claimed is:

1. A compound of Formula I, Formula II, or Formula III: 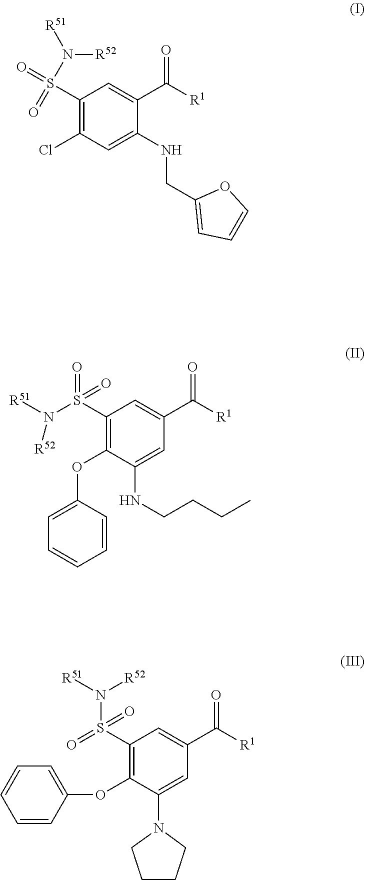
or a pharmaceutically acceptable salt thereof
wherein:
R¹ is selected from:
(i) —OC₁₅-C₃₀alkylR³, —OC₂-C₃₀alkenylR³, —OC₂-C₃₀alkynylR³, —OC₄-C₃₀alkenylalkynylR³, —OC₁₅-C₃₀alkyl, —OC₂-C₃₀alkenyl, and —OC₂-C₃₀alkynyl, and —OC₄-C₃₀alkenylalkynyl;
(ii) —OC₁₅₋₃₀alkyl with at least one R³ substituent on the alkyl chain, —OC₁₋₃₀alkenyl with at least one R³ substituent on the alkenyl chain, and —OC₁₋₃₀alkynyl with at least one R³ substituent on the alkynyl chain;
(iii) —(OCH₂C(O))₁₋₂₀OC₁₋₃₀alkyl, —(OCH(CH₃)C(O))₁₋₂₀OC₁₋₃₀alkyl, —(OCH₂C(O))₁₋₁₀OC₁₋₃₀alkyl, —(OCH(CH₃)C(O))₁₋₁₀OC₁₋₃₀alkyl, —(OCH₂C(O))₄₋₂₀OC₁₋₃₀alkyl, —(OCH(CH₃)C(O))₄₋₂₀OC₁₋₃₀alkyl, —(OCH₂C(O))₄₋₂₀OC₁₋₁₀alkyl, —(OCH(CH₃)C(O))₁₋₂₀OC₁₋₁₀alkyl, —(OCH₂C(O))₁₋₂₀OC₄₋₁₀alkyl, —(OCH(CH₃)C(O))₁₋₂₀OC₄₋₁₀alkyl, —(OCH₂C(O))₁₋₂₀OH, —(OCH(CH₃)C(O))₁₋₂₀OH, —(OCH₂C(O))₁₋₁₀OH, —(OCH(CH₃)C(O))₁₋₁₀OH, —(OCH₂C(O))₄₋₂₀OH, —(OCH(CH₃)C(O))₄₋₂₀OH, —(OCH₂C(O))₄₋₁₀OH, —(OCH(CH₃)C(O))₄₋₁₀OH, —(OCH(CH₃)C(O))₄₋₁₀OC₁₋₁₀alkyl, —(OCH₂C(O))₄₋₁₀OC₁₋₁₀alkyl, —(OCH(CH₃)C(O))₁₋₁₀OC₁₋₁₀alkyl, —(OCH₂C(O))₁₋₁₀OC₁₋₁₀alkyl, —(OCH(CH₃)C(O))₁₋₁₀OC₄₋₁₀alkyl, —(OCH₂C(O))₁₋₁₀OC₄₋₁₀alkyl, —(OCH₂C(O))₁₋₁₀OC₄₋₁₀alkyl, —(OCH(CH₃)C(O))₁₋₁₀OC₄₋₁₀alkyl, —(OCH₂C(O))₁₋₁₀OC₄₋₁₀alkyl, —(OCH(CH₃)C(O))₁₋₁₀OC₄₋₁₀alkyl, —(OCH₂C(O))₁₋₁₀(OCH(CH₃)C(O))₁₋₁₀OC₁₋₃₀alkyl, —(OCH₂C(O))₂₋₁₀(OCH(CH₃)C(O))₂₋₁₀OC₁₋₃₀alkyl, —(OCH₂C(O))₁₋₁₀(OCH(CH₃)C(O))₁₋₁₀OC₁₋₁₂alkyl, —(OCH₂C(O))₁₋₁₀(OCH(CH₃)C(O))₁₋₁₀OC₄₋₂₂alkyl, —(OCH(CH₃)C(O))₁₋₁₀(OCH₂C(O))₁₋₁₀OC₁₋₃₀alkyl, —(OCH(CH₃)C(O))₂₋₁₀(OCH₂C(O))₂₋₁₀OC₁₋₃₀alkyl, —(OCH(CH₃)C(O))₁₋₀(OCH₂C(O))₁₋₁₀OC₁₋₁₂alkyl, and —(OCH(CH₃)C(O))₁₋₁₀(OCH₂C(O))₁₋₁₀OC₄₋₂₂alkyl;
(iv) polylactic acid, poly(lactic-co-glycolic acid), polyglycolic acid, 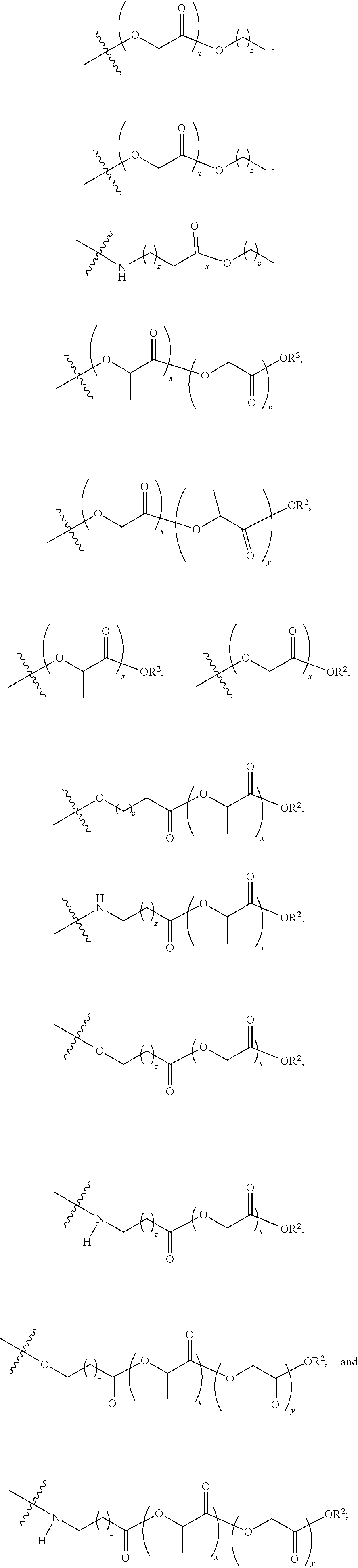
and
(v) —OH;
wherein R¹ cannot be OH when R⁵¹ and R⁵² are both hydrogen;
R² is selected at each instance from alkyl, alkenyl, alkynyl, cycloalkyl, cycloalkylalkyl, heterocyclyl, heterocycloalkyl, aryl, arylalkyl, heteroaryl, and heteroarylalkyl;
R³ is selected from halogen, hydroxyl, cyano, mercapto, amino, alkoxy, cycloalkyl, cycloalkylalkyl, heterocyclyl, heterocycloalkyl, aryl, arylalkyl, heteroaryl, heteroarylalkyl, aryloxy, —S(O)₂alkyl, —S(O)alkyl, —P(O)(Oalkyl)₂, B(OH)₂, —Si(CH₃)₃, —COOH, —COOalkyl, and —CONH₂;
R⁵¹ and R⁵² are independently selected from hydrogen, 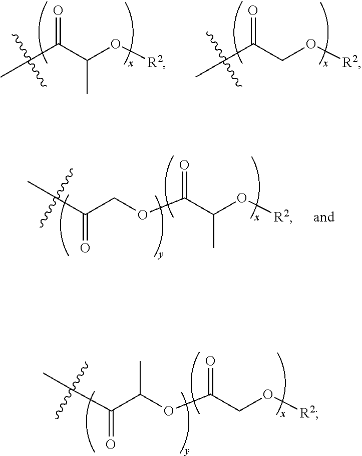
x and y at each instance are independently selected from 1, 2, 3, 4, 5, 6, 7, 8, 9, 10, 11, and 12; and
z is independently selected from 0, 1, 2, 3, 4, 5, 6, 7, 8, 9, 10, 11, and 12.

2. The compound of claim 1 selected from 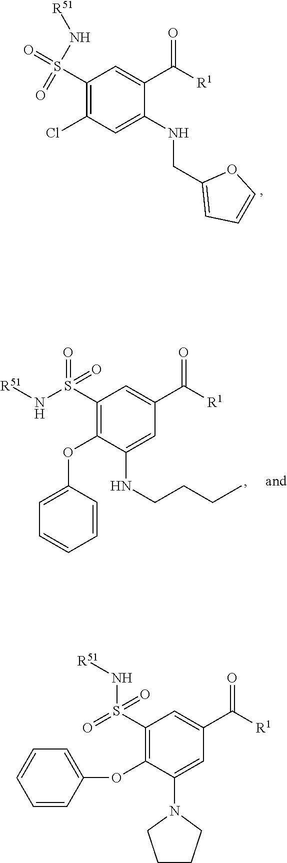
or a pharmaceutically acceptable salt thereof.

3. The compound of claim 1 selected from 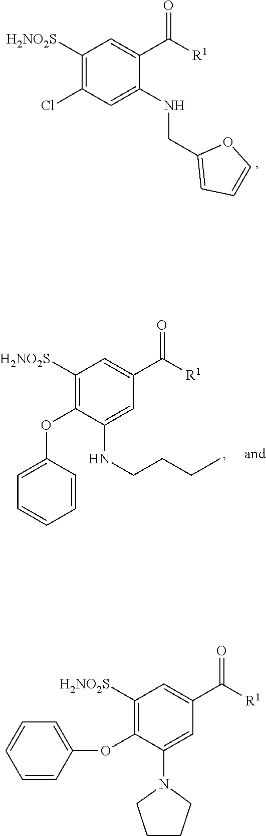
or a pharmaceutically acceptable salt thereof.

4. The compound of claim 1, wherein R¹ is selected from

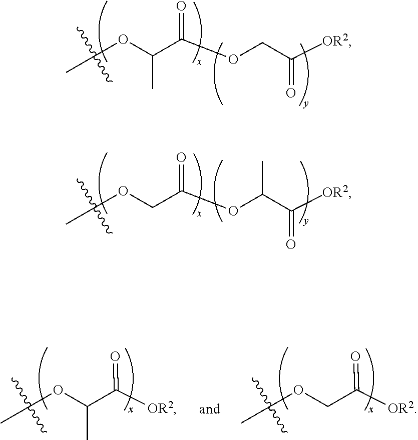

5. The compound of claim 1, wherein R¹ is selected from

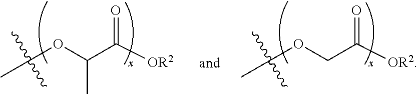

6. The compound of claim 5, wherein R¹ is selected from

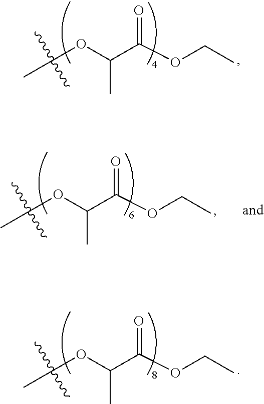

7. The compound of claim 1, wherein R² is selected from hydrogen, alkyl, aryl, and arylalkyl.

8. The compound of claim 7, wherein R² is alkyl and alkyl is ethyl.

9. The compound of claim 1, wherein R⁵¹ and R⁵² are hydrogen.

10. The compound of claim 1, wherein R⁵¹ and R⁵² are selected from

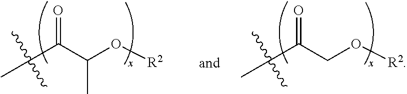

11. The compound of claim 1 selected from the formula: 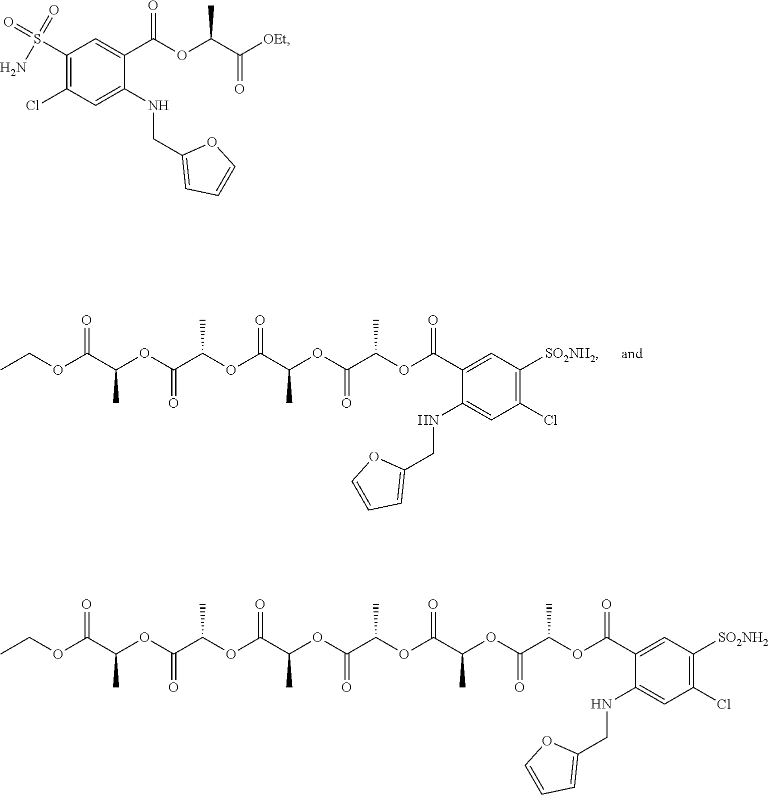 [SMILES: CCOC(=O)[C@H](C)OC(=O)c1cc(S(N)(=O)=O)c(Cl)cc1NCc1ccco1 ; CCOC(=O)[C@H](C)OC(=O)[C@H](C)OC(=O)[C@H](C)OC(=O)[C@H](C)OC(=O)c1cc(S(N)(=O)=O)c(Cl)cc1NCc1ccco1 ; "and" ; CCOC(=O)[C@H](C)OC(=O)[C@H](C)OC(=O)[C@H](C)OC(=O)[C@H](C)OC(=O)[C@H](C)OC(=O)[C@H](C)OC(=O)c1cc(S(N)(=O)=O)c(Cl)cc1NCc1ccco1]
or a pharmaceutically acceptable salt thereof.

12. The compound of claim 1 of the formula: 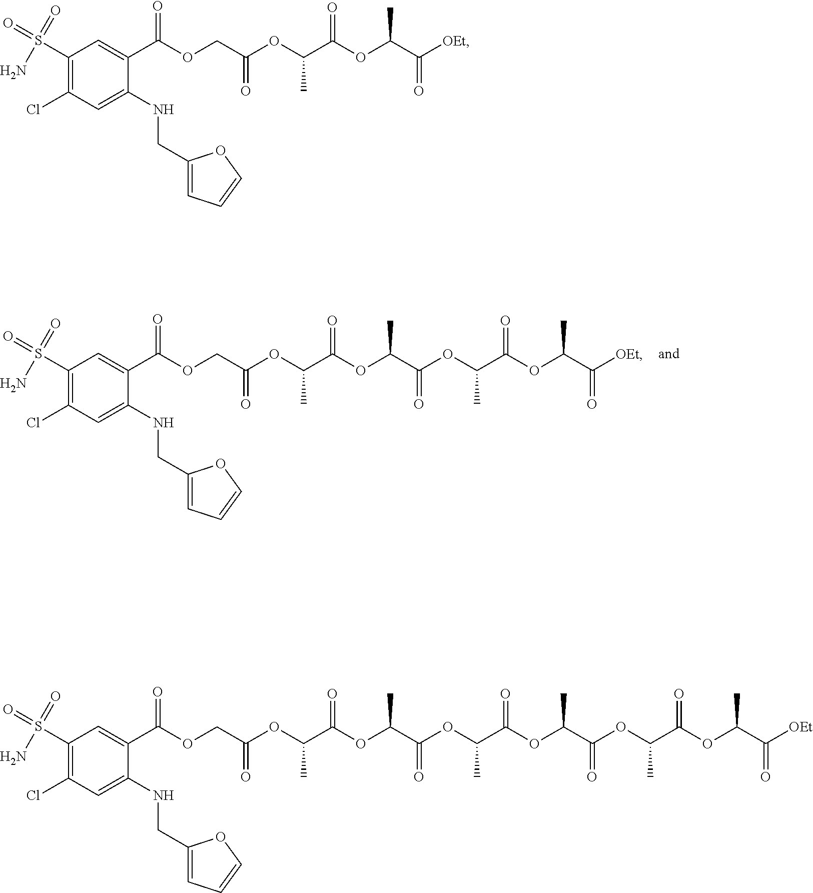 [SMILES: CCOC(=O)[C@H](C)OC(=O)[C@H](C)OC(=O)COC(=O)c1cc(S(N)(=O)=O)c(Cl)cc1NCc1ccco1 ; CCOC(=O)[C@H](C)OC(=O)[C@H](C)OC(=O)[C@H](C)OC(=O)[C@H](C)OC(=O)COC(=O)c1cc(S(N)(=O)=O)c(Cl)cc1NCc1ccco1 ; CCOC(=O)[C@H](C)OC(=O)[C@H](C)OC(=O)[C@H](C)OC(=O)[C@H](C)OC(=O)[C@H](C)OC(=O)[C@H](C)OC(=O)COC(=O)c1cc(S(N)(=O)=O)c(Cl)cc1NCc1ccco1]
or a pharmaceutically acceptable salt thereof.

13. The compound of claim 1 selected from the formula: 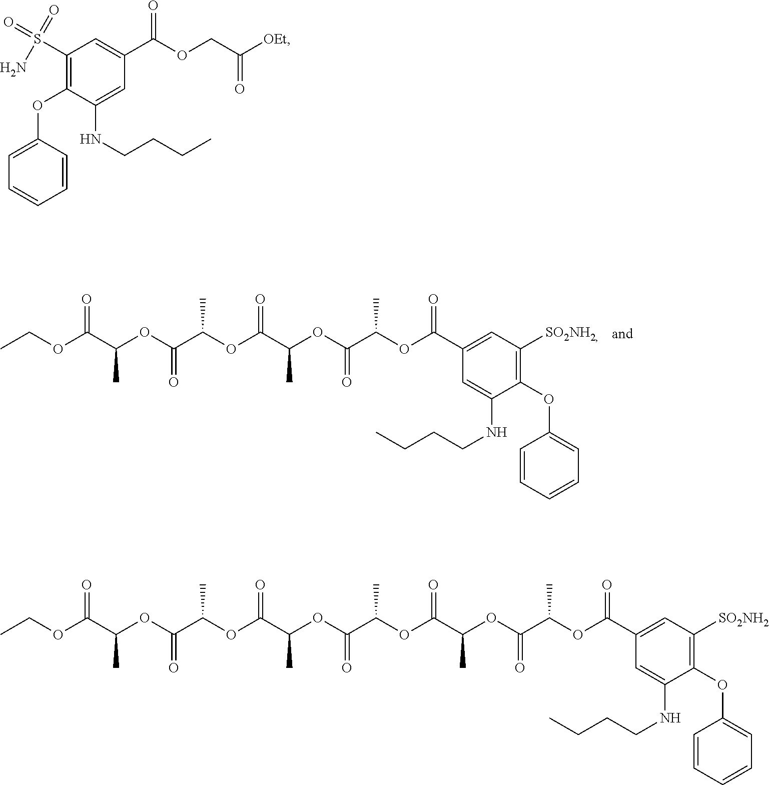 [SMILES: CCCCNc1cc(C(=O)OCC(=O)OCC)cc(S(N)(=O)=O)c1Oc1ccccc1 ; CCCCNc1cc(C(=O)O[C@@H](C)C(=O)O[C@@H](C)C(=O)O[C@@H](C)C(=O)O[C@@H](C)C(=O)OCC)cc(S(N)(=O)=O)c1Oc1ccccc1 ; CCCCNc1cc(C(=O)O[C@@H](C)C(=O)O[C@@H](C)C(=O)O[C@@H](C)C(=O)O[C@@H](C)C(=O)O[C@@H](C)C(=O)O[C@@H](C)C(=O)OCC)cc(S(N)(=O)=O)c1Oc1ccccc1]
or a pharmaceutically acceptable salt thereof.

14. The compound of claim 1 selected from the formula: 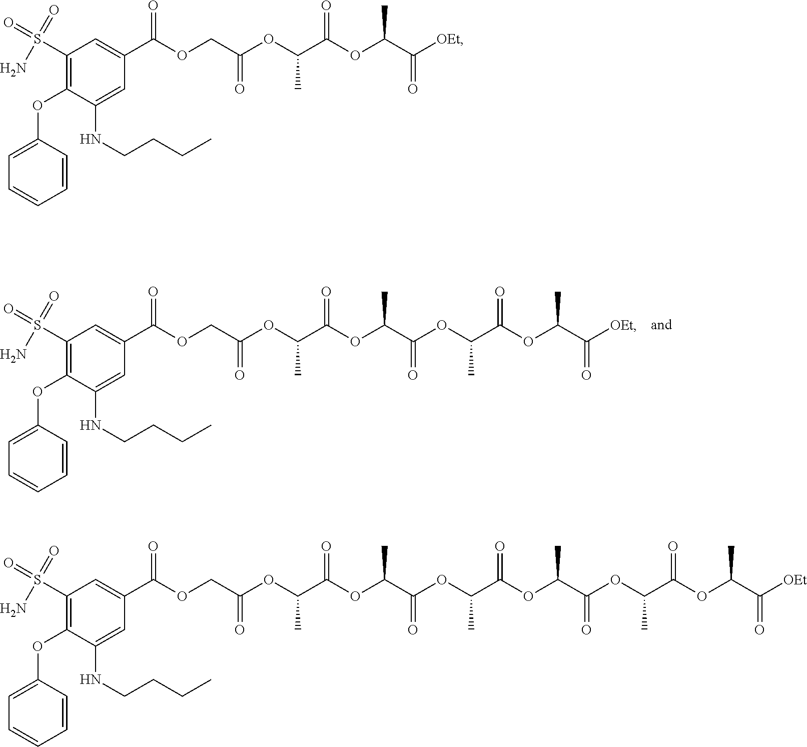 [SMILES: CCCCNc1cc(C(=O)OCC(=O)O[C@@H](C)C(=O)O[C@@H](C)C(=O)OCC)cc(S(N)(=O)=O)c1Oc1ccccc1 ; CCCCNc1cc(C(=O)OCC(=O)O[C@@H](C)C(=O)O[C@@H](C)C(=O)O[C@@H](C)C(=O)O[C@@H](C)C(=O)OCC)cc(S(N)(=O)=O)c1Oc1ccccc1 ; CCCCNc1cc(C(=O)OCC(=O)O[C@@H](C)C(=O)O[C@@H](C)C(=O)O[C@@H](C)C(=O)O[C@@H](C)C(=O)O[C@@H](C)C(=O)O[C@@H](C)C(=O)OCC)cc(S(N)(=O)=O)c1Oc1ccccc1]
or a pharmaceutically acceptable salt thereof.

15. The compound of claim 1 selected from the formula: 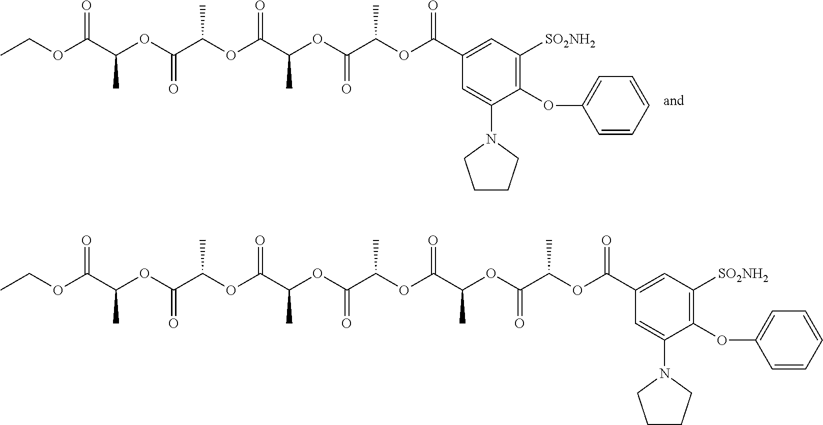 [SMILES: CCOC(=O)[C@H](C)OC(=O)[C@H](C)OC(=O)[C@H](C)OC(=O)[C@H](C)OC(=O)c1cc(N2CCCC2)c(Oc2ccccc2)c(S(N)(=O)=O)c1 ; CCOC(=O)[C@H](C)OC(=O)[C@H](C)OC(=O)[C@H](C)OC(=O)[C@H](C)OC(=O)[C@H](C)OC(=O)[C@H](C)OC(=O)c1cc(N2CCCC2)c(Oc2ccccc2)c(S(N)(=O)=O)c1]
or a pharmaceutically acceptable salt thereof.

16. A pharmaceutical composition comprising a compound of claim 1, optionally in a pharmaceutically acceptable carrier.

17. A method for the treatment of an ocular disorder in a host in need thereof comprising administering an effective amount of a compound of claim 1 optionally in a pharmaceutically acceptable carrier.

18. The method of claim 17, wherein the host is a human.

19. The method of claim 18, wherein the ocular disorder is selected from glaucoma, wet age-related macular degeneration, dry age-related macular degeneration, a disorder related to an increase in intraocular pressure (IOP), a disorder mediated by nitric oxide synthase (NOS), optic nerve damage caused by high intraocular pressure (IOP), a disorder requiring neuroprotection, or diabetic retinopathy.

20. The method of claim 19, wherein the compound is administered via intravitreal, intrastromal, intracameral, sub-tenon, sub-retinal, retro-bulbar, peribulbar, suprachoroidal, choroidal, subchoroidal, conjunctival, subconjunctival, episcleral, posterior juxtascleral, circumcorneal, or tear duct injection.

21. The method of claim 20, wherein the compound is administered via intravitreal injection.

22. The method of claim 20, wherein the compound is administered via suprachoroidal injection.

23. The method of claim 20, wherein the compound is administered via subconjunctival injection.
